(19) United States
(12) Patent Application Publication
(10) Pub. No.: US 2023/0364226 A1
(43) Pub. Date: Nov. 16, 2023

(54) RNA CONSTRUCT

(71) Applicants: Imperial College Innovations Limited, London (GB); VaxEquity Ltd, Little Chesterford (GB)

(72) Inventors: Robin Shattock, London (GB); Paul McKay, London (GB); Michael Watson, Little Chesterford (GB); Elaine Harper, Little Chesterford (GB)

(73) Assignees: Imperial College Innovations Limited, London (GB); VaxEquity Ltd, Little Chesterford, ES (GB)

(21) Appl. No.: 18/257,548

(22) PCT Filed: Dec. 17, 2021
(86) PCT No.: PCT/GB2021/053362

§ 371 Date: Jun. 14, 2023

(30) Foreign Application Priority Data
Dec. 17, 2020 (GB) 2020061.4

(51) Int. Cl. A61K 39/39; C12N 15/86; C07K 14/005

(52) U.S. Cl. CPC A61K 39/39; C12N 15/86; C07K 14/005; A61K 2039/53

(57) ABSTRACT

The invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

The present invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

Messenger RNA (mRNA) is a promising tool for biotherapeutics. However, while mRNA therapeutics have been shown to be highly effective in small animals, the outcomes do not scale linearly when these formulations are translated in dose-escalation studies in humans. Furthermore, adverse events associated with the induction of interferon responses have been rate-limiting with respect to the increased doses of RNA likely to be effective in humans. The reason for this inconsistency is unclear, but the inventors hypothesize that inherent differences in human innate sensing pose a barrier to the translation of RNA therapeutics from the lab to the clinic.

Furthermore, innate sensing of RNA has been associated with the inhibition of protein expression. To date, the main approach to overcoming the innate recognition of exogenous RNA has been to use modified ribonucleotides that are less detectable by innate sensing mechanisms. However, modified mRNA is not completely undetectable, and still results in some induction of interferon production, protein silencing and reduced tolerability for human use (see FIG. 2).

Another approach has been the use of self-amplifying or saRNA vectors, which are typically based on an alphavirus backbone that have the capacity to self-amplify their own RNA by encoding polymerase activity within their non-structural proteins. Prior art methods have involved replacing the structural proteins of these vectors by a gene of interest (GOI), for example encoding an antigen of interest be it a vaccine construct, or encoding a therapeutic protein. Other versions of saRNA have been based on picornaviruses, flaviviruses, and coronaviruses. When saRNA is taken up into the cytoplasm of target cells, this leads to amplification of the RNA by the encoded polymerase machinery and very high expression levels of the GOI. As a consequence, saRNA has been shown to induce immune responses with lower doses of saRNA than mRNA (10- to 100-fold lower) and results in prolonged protein expression for up to 60 days in mice.

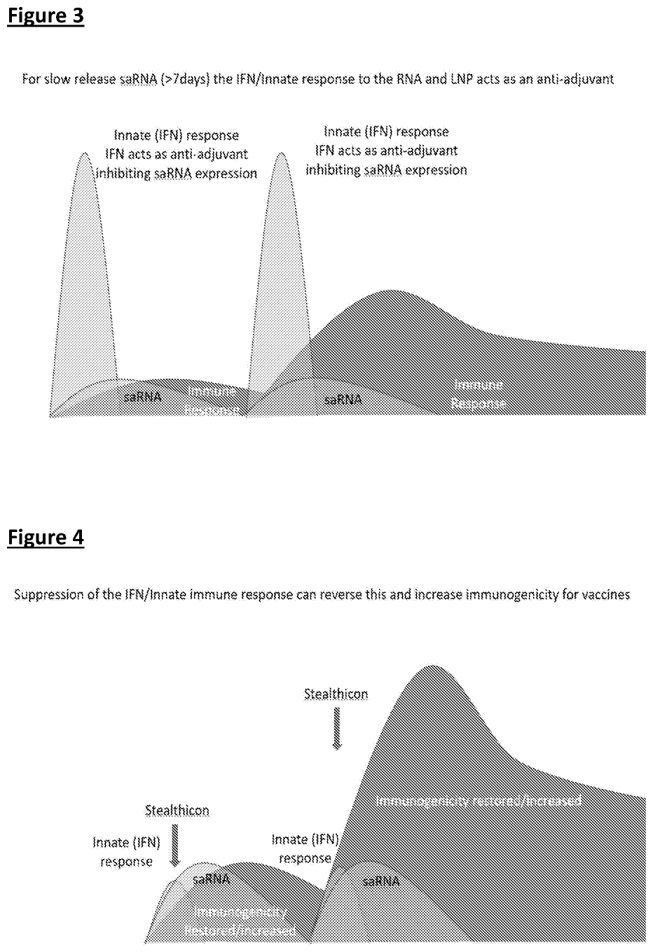

However, as shown in FIG. 3, a drawback with saRNA is that it is also sensed by innate sensing pattern recognition receptors, triggering antiviral (interferon) responses that limit protein expression and self-amplification of these prior art saRNAs. Innate sensing of saRNA differs to that of mRNA due to its large size (typically >5000 bases) and profound secondary structure, including double stranded regions (dsRNA). Long and double stranded RNA triggers innate responses through amongst other sensors, the MDA5 ((Melanoma Differentiation-Associated protein 5) pathway. This is facilitated by the binding of PACT (PKR activating protein) to long and dsRNA RNA promoting the oligomerization of MDA5 and subsequent triggering of a down-stream signalling cascade that inhibits replication and expression of saRNA.

Accordingly, there is a need in the art to produce new means by which RNA therapeutics, be they mRNA- or saRNA-based, can be delivered and expressed in patients, such that they are able to overcome the innate immune system sensing.

The inventors have developed novel RNA constructs (saRNA and mRNA) that advantageously overcome the innate immune system which senses RNA, by expressing viral immune inhibitor proteins that block or reduce the activity of immune system machinery, resulting in improved translation (in the case of mRNA) and increased self-amplification and subsequent translation (in the case of saRNA systems), and therefore greater protein expression levels of the gene of interest, such as an antigen, in a host cell.

Accordingly, in a first aspect of the invention, there is provided an RNA construct encoding: (i) at least one therapeutic biomolecule; and (ii) at least one viral innate inhibitor protein (IIP).

RNA constructs, such as mRNA and saRNA replicons, have been postulated to be potential tools for the delivery and expression of genes of interest for vaccines and therapeutics. However, single stranded mRNA (ssRNA) and double stranded RNA (dsRNA) is detected intracellularly by innate sensing mechanisms that trigger responses, which inhibit protein translation. As a consequence, expression of genes of interest encoded by the RNA construct is significantly impaired and thus the immunogenic or therapeutic potential of RNA constructs, including saRNA and mRNA, is limited. Advantageously, the RNA constructs of the invention overcome this problem because they encode one or more viral innate inhibitor protein (IIP), which reduces or ablates the downstream innate inhibition of transgene expression within the host cell.

The induction of interferon is one downstream consequence of innate recognition, but it will be appreciated that other molecules and pathways can and are induced, as discussed below, and any of these will be inhibited by the one or more viral innate immunity inhibitor protein that is harboured on the RNA construct. Preferably, therefore, the at least one innate inhibitor protein (IIP) is capable of inhibiting the innate immune response to RNA in a subject treated with the RNA construct of the invention. The IIP can therefore be described as an inhibitor of innate immunity. It may also be described as an interferon inhibiting molecule in some embodiments.

One previously published approach to ablating the interferon response with saRNA used interferon inhibiting proteins from the vaccinia virus, E3, K3 and B18. However, in that study, the interferon inhibiting proteins were delivered and formulated as separate mRNA molecules that were combined with the saRNA. This requires the manufacture of both saRNA and mRNA, and necessitated the use of at least 3-6 times as much vaccinia mRNA as the saRNA replicon construct according to the invention to provide any observable enhancement in protein expression.

Advantageously, the presence, in the RNA construct of the first aspect, of one or more viral innate inhibitor protein, enables dual protein expression with the biotherapeutic molecule, i.e. a peptide or protein of interest. As opposed to delivering two different strands of RNA as described in the prior art, one encoding the peptide/protein of interest and one encoding the innate modulatory protein, when using the RNA construct of the invention, only one single strand is delivered to the target cell, thereby ensuring colocalization of the RNA molecule and the viral immune inhibitor protein.

The viral immune inhibitor protein inhibits the innate sensing of RNA in the host cell, thereby enabling higher protein expression and translation, and the viral immune inhibitor protein expression itself is co-expressed and translated from the same RNA molecule as the therapeutic biomolecule.

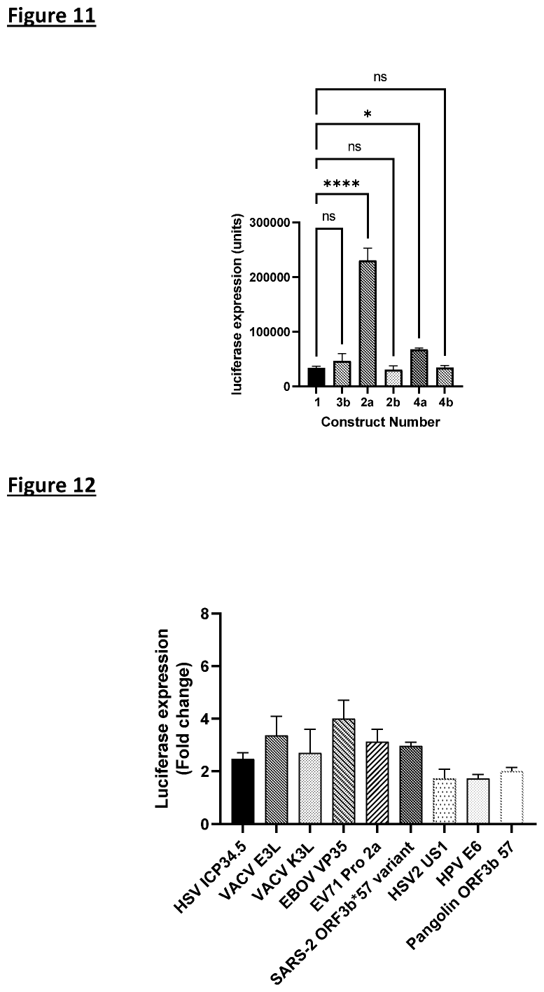

As described in the examples, the RNA constructs of the invention (also known as “Stealthicons”) encoding luciferase or VEGF-A (as a GOI) have surprisingly been shown to increase luciferase or VEGF-A protein expression levels by up to 12-fold in a human cell line with intact innate sensing systems in vitro. The skilled person would readily appreciate that the luciferase reporter is truly representative of the therapeutic biomolecule described herein (i.e. the GOI), because it proves that the RNA construct is able to express the gene harboured on the RNA molecule of the invention. As such, the luciferase provides robust evidence of the proof of concept that the RNA construct of the invention can be used to express any therapeutically active biomolecule. In addition, VEGF-A (see FIG. 11) represents an alternative exemplar to luciferase as the GOI.

The RNA construct of the first aspect may be single-stranded RNA or double-stranded RNA.

The RNA construct may comprise a mRNA molecule or a saRNA molecule.

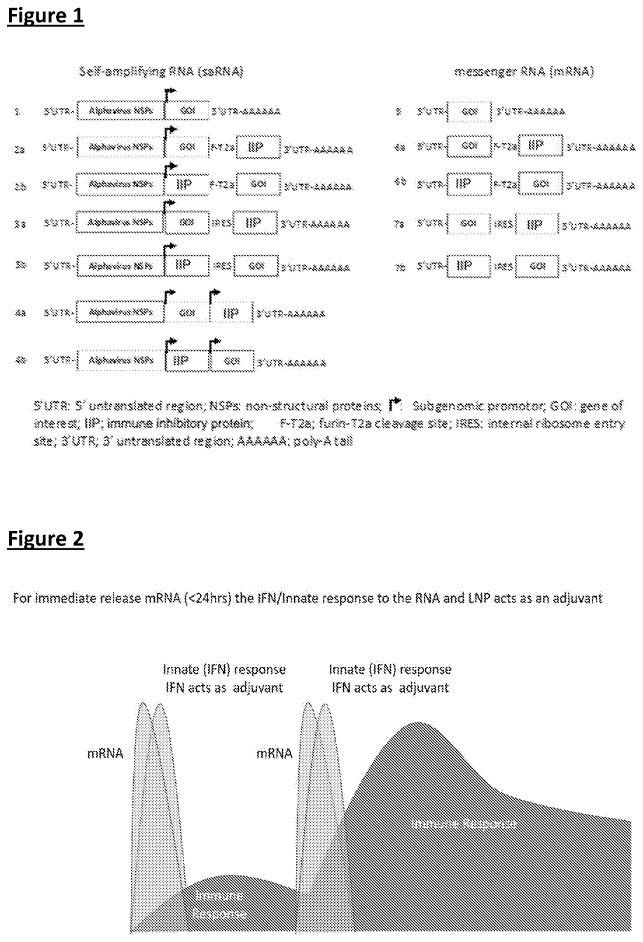

In one embodiment, the RNA construct comprises mRNA. FIG. 1 (right hand side) illustrates various embodiments of the RNA construct as a mRNA molecule.

In a preferred embodiment, however, the RNA construct comprises self-amplifying RNA (saRNA). FIG. 1 (left hand side) illustrates various embodiments of the RNA construct as a saRNA molecule. The skilled person would understand that such an RNA construct can also be referred to as a self-replicating RNA virus vector, or an RNA replicon.

Preferably, the saRNA construct comprises or is derived from a positive stranded RNA virus selected from the group of genus consisting of: alphavirus; picornavirus; flavivirus; rubivirus; pestivirus; hepacivirus; calicivirus and coronavirus.

Preferably, the RNA construct comprises or is derived from an alphavirus. Suitable wild-type alphavirus sequences are well-known. Representative examples of suitable alphaviruses include Aura, Bebaru virus, Cabassou, Chikungunya virus, Eastern equine encephalomyelitis virus, Fort Morgan, Getah virus, Kyzylagach, Mayaro, Mayaro virus, Middleburg, Mucambo virus, Ndumu, Pixuna virus, Ross River virus, Semliki Forest, Sindbis virus, Tonate, Triniti, Una, Venezuelan equine encephalomyelitis, Western equine encephalomyelitis, Whataroa, and Y-62-33. Preferably, therefore, the RNA construct comprises or is derived from any of these alphaviruses.

Preferably, the RNA construct comprises or is derived from a virus selected from the group of species consisting of: Venezuelan Equine Encephalitis Virus (VEEV); enterovirus 71; Encephalomyocarditis virus; Kunjin virus; and Middle East respiratory syndrome virus. In one preferred embodiment, the RNA construct comprises or is derived from Kunjin virus. Preferably, the RNA construct comprises or is derived from VEEV.

Preferably, the RNA construct comprises a nucleotide sequence, which encodes the at least one innate inhibitor protein (IIP), which is capable of reducing, ablating or blocking the innate immune response to RNA. The IIP is, therefore, an inhibitor of innate immunity, and can therefore be described as a viral innate immunity inhibitor protein. The IIP may also be an inhibitor of interferon signalling.

The reduction, ablation or blocking of the innate immune response to RNA in a host cell transformed with that RNA molecule (i.e. non-endogenously produced RNA) may be achieved by the IIP regulating interferon production, inhibiting innate signalling pathways, and/or inhibiting RNA recognition. It will be appreciated that regulation of interferon production could be described as inhibiting innate signalling. Therefore, innate sensing and innate signalling systems include: (a) RNA recognition systems, (b) pathways leading to interferon production and resulting in stimulation of interferon-stimulated genes, and (c) interferon signalling systems.

The IIP may, therefore, fall into any of the following four broad categories:—
(i) Category 1: Inhibitors of interferon regulatory factor activity;
(ii) Category 2: Inhibitors of pathways leading to interferon production and resulting in stimulation of interferon-stimulated genes;
(iii) Category 3: Inhibitors of interferon signalling; and/or
(iv) Category 4: Inhibitors of RNA recognition systems.

It will be appreciated that some IIPs may have multiple actions. For instance, a Category 4 IIP may also be classified as a Category 2 IIP (e.g. IRF3, IRF7) and a Category 3 IIP (e.g. IRF9).

In one embodiment, the reduction, ablation or blocking of the innate immune response to RNA is preferably achieved by the IIP by reducing or preventing the recognition of cytosolic RNA by pattern recognition receptors leading to activation of interferon regulatory factor 3 and 7 (IRF3 and IRF7) and NF-κB transcription factors, directly triggering a range of antiviral genes (e.g. IFIT1-3, Mx1, Mx2 known to suppress RNA expression), proinflammatory genes whose products orchestrate the innate immune response, and direct activation of canonically IFN-stimulated genes (ISGs) upstream of any interferon dependent cascade. These pathways may be enhanced by the induction of type I & III interferons that provide a positive feedback loop further amplifying many antiviral responses.

The at least one IIP may be derived from a herpes simplex virus. The IIP may be derived from a hepatitis virus, optionally a hepatitis C virus. The IIP may be derived from a vaccinia virus. The IIP may be derived from a coronavirus, optionally Middle East Respiratory Syndrome (MERS) virus or Severe Acute Respiratory Syndrome (SARS) virus. The SARS virus may be SARS-Cov or SARS-CoV-2. The IIP may be derived from a Karposi's sarcoma-associated herpesvirus (KSHV). The IIP may be derived from an Ebola virus.

The at least one innate inhibitor protein (IIP) may be selected from a group of viral IIPs consisting of: HPV16 E6; HSV ICP34.5; HCV E2; HCV NS5a; VACV E3L; VACV K3L; MERS ORF8B; KSHV ORF52; EBOV VP35; SARS-2 ORF3b and VACV C6 or an orthologue thereof. It will be appreciated that these IIPs can bring about inhibition of innate signalling systems leading to IFN production.

In one embodiment, the at least one IIP may be HPV16 E6 (human papillomavirus E6; NP_041325.1; Accession Number—NCBI Reference Sequence: NC_001526.4; UniProtKB—P03126 (VE6_HPV16)), or an orthologue thereof. This protein is believed to prevent the establishment of a cellular antiviral state by acting on multiple proteins within the innate signalling cascade including PKR, IRF3 and IRF9. Westrich J A, Warren C J, Pyeon D. (2017). Evasion of host immune defenses by human papillomavirus. Virus Res. 2017 231, 21-33. doi: 10.1016/j.virusres.2016.11.023. Noguchi T, Satoh, S, Noshi T, Hatada E, Fukuda R, Kawai A, Ikeda S, Hijikata M. Shimotohno K (2001), Effects of Mutation in Hepatitis C Virus Nonstructural Protein 5A on Interferon Resistance Mediated by Inhibition of PKR Kinase Activity in Mammalian Cells, Microbiol. Immunol., 45-840. doi: 10.1111/j.1348-0421.2001.tbo1322.x.).

One embodiment of the HPV16 E6 polypeptide sequence is represented herein as SEQ ID No: 1, as follows:

[SEQ ID No: 1]
MHQKRTAMFQDPQERPRKLPQLCTELQTTIHDIILECVYCKQQLLRR
EVYDFAFRDLCIVYRDGNPYAVCDKCLKFYSKISEYRHYCYSLYGTT
LEQQYNKPLCDLLIRCINCQKPLCPEEKQRHLDKKQRFHNIRGRWTG
RCMSCCRSSRTRRETQL

Therefore, preferably the RNA construct of the first aspect comprises a nucleotide sequence which encodes an amino acid sequence substantially as set out in SEQ ID No: 1, or a variant or fragment thereof.

In one embodiment, the HPV16 E6 polypeptide is encoded by the DNA nucleotide sequence of SEQ ID No: 2, as follows:

[SEQ ID No: 2]
ATGCACCAAAAGAGAACTGCAATGTTTCAGGACCCACAGGAGCGACC
CAGAAAGTTACCACAGTTATGCACAGAGCTGCAAACAACTATACATG
ATATAATATTAGAATGTGTGTACTGCAAGCAACAGTTACTGCGACGT
GAGGTATATGACTTTGCTTTTCGGGATTTATGCATAGTATATAGAGA
TGGGAATCCATATGCTGTATGTGATAAATGTTTAAAGTTTTATTCTA
AAATTAGTGAGTATAGACATTATTGTTATAGTTTGTATGGAACAACA
TTAGAACAGCAATACAACAAACCGTTGTGTGATTTGTTAATTAGGTG
TATTAACTGTCAAAAGCCACTGTGTCCTGAAGAAAAGCAAAGACATC
TGGACAAAAAGCAAAGATTCCATAATATAAGGGGTCGGTGGACCGGT
CGATGTATGTCTTGTTGCAGATCATCAAGAACACGTAGAGAAACCCA
GCTG

Accordingly, preferably the HPV16 E6 polypeptide is encoded by the DNA nucleotide sequence substantially as set out in SEQ ID NO: 2, or a variant or fragment thereof.

Thus, the RNA construct may comprise an RNA nucleotide sequence of SEQ ID No: 3, as follows:

[SEQ ID No: 3]
AUGCACCAAAAGAGAACUGCAAUGUUUCAGGACCC
ACAGGAGCGACCCAGAAAGUUACCACAGUUAUGCA
CAGAGCUGCAAACAACUAUACAUGAUAUAAUAUUA
GAAUGUGUGUACUGCAAGCAACAGUUACUGCGACG
UGAGGUAUAUGACUUUGCUUUUCGGGAUUUAUGCA
UAGUAUAUAGAGAUGGGAAUCCAUAUGCUGUAUGU
GAUAAAUGUUUAAAGUUUUAUUCUAAAAUUAGUGA
GUAUAGACAUUAUUGUUAUAGUUUGUAUGGAACAA
CAUUAGAACAGCAAUACAACAAACCGUUGUGUGAU
UUGUUAAUUAGGUGUAUUAACUGUCAAAAGCCACU
GUGUCCUGAAGAAAAGCAAAGACAUCUGGACAAAA
AGCAAAGAUUCCAUAAUAUAAGGGGUCGGUGGACC
GGUCGAUGUAUGUCUUGUUGCAGAUCAUCAAGAAC
ACGUAGAGAAACCCAGCUG

Furthermore, preferably the RNA construct comprises an RNA nucleotide sequence substantially as set out in SEQ ID No: 3, or a variant or fragment thereof.

The inventors then subjected the protein sequence of SEQ ID No: 1 to codon optimisation for human expression, and one embodiment of the codon optimised nucleic acid (DNA) sequence that includes a start (ATG) and a stop (TGA) codon is provided herein as SEQ ID No: 4, as follows:

[SEQ ID No: 4]
ATGCACCAGAAACGGACCGCCATGTTCCAGGATCC
TCAAGAGAGGCCCAGAAAGCTGCCTCAGCTGTGTA
CCGAGCTGCAGACCACCATCCACGACATCATCCTG
GAATGCGTGTACTGCAAGCAGCAGCTCCTGCGGAG
AGAGGTGTACGATTTCGCCTTCCGGGACCTGTGCA
TCGTGTACAGAGATGGCAACCCCTACGCCGTGTGC
GACAAGTGCCTGAAGTTCTACAGCAAGATCAGCGA
GTACCGGCACTACTGCTACAGCCTGTACGGCACCA
CACTGGAACAGCAGTACAACAAGCCCCTGTGCGAC
CTGCTGATCCGGTGCATCAACTGCCAGAAACCTCT
GTGCCCCGAGGAAAAGCAGCGGCACCTGGACAAGA
AGCAGCGGTTCCACAACATCAGAGGCCGGTGGACC
GGCAGATGCATGAGCTGTTGTCGGAGCAGCAGAAC
CAGACGGGAAACCCAGCTGTGA

Hence, preferably the RNA construct is encoded by a DNA sequence substantially as set out in SEQ ID No: 4, or a fragment or variant thereof.

In an embodiment, the RNA sequence corresponding to the codon optimised DNA sequence of SEQ ID No: 4 that includes a start (AUG) and a stop (UGA) codon is provided herein as SEQ ID No: 5, as follows:

[SEQ ID No: 5]
AUGCACCAGAAACGGACCGCCAUGUUCCAGGAUCC
UCAAGAGAGGCCCAGAAAGCUGCCUCAGCUGUGUA
CCGAGCUGCAGACCACCAUCCACGACAUCAUCCUG
GAAUGCGUGUACUGCAAGCAGCAGCUCCUGCGGAG
AGAGGUGUACGAUUUCGCCUUCCGGGACCUGUGCA
UCGUGUACAGAGAUGGCAACCCCUACGCCGUGUGC
GACAAGUGCCUGAAGUUCUACAGCAAGAUCAGCGA
GUACCGGCACUACUGCUACAGCCUGUACGGCACCA
CACUGGAACAGCAGUACAACAAGCCCCUGUGCGAC
CUGCUGAUCCGGUGCAUCAACUGCCAGAAACCUCU
GUGCCCCGAGGAAAAGCAGCGGCACCUGGACAAGA
AGCAGCGGUUCCACAACAUCAGAGGCCGGUGGACC
GGCAGAUGCAUGAGCUGUUGUCGGAGCAGCAGAAC
CAGACGGGAAACCCAGCUGUGA

Hence, preferably the RNA construct comprises a sequence substantially as set out in SEQ ID No: 5, or a fragment or variant thereof.

In one embodiment, the at least one IIP may be HSV ICP34.5 (Herpes simplex virus ICP34.5; YP_009137073.1; Accession Number—NCBI Reference Sequence: NC_001806.2; UniProtKB—P36313 (ICP34_HHV11)), or an orthologue thereof. HSV ICP34.5 has been shown to sequester TBK1 and interfere with the interaction between TBK1 and IRF3, ultimately leading to the inactivation of IRF3. Zhu H, Zheng C (2020).

The race between host antiviral innate immunity and the immune evasion strategies of Herpes simplex virus 1. Microbiol Mol Biol Rev., 84(4): e00099-20. One embodiment of the HSV ICP34.5 polypeptide sequence is represented herein as SEQ ID No:6, as follows:

[SEQ ID No: 6]
MARRRRHRGPRRPRPPGPTGAVPTAQSQVTSTPNS
EPAVRSAPAAAPPPPPAGGPPPSCSLLLRQWLHVP
ESASDDDDDDDWPDSPPPEPAPEARPTAAAPRPRP
PPPGVGPGGGADPSHPPSRPFRLPPRLALRLRVTA
EHLARLRLRRAGGEGAPEPPATPATPATPATPATP
ARVRFSPHVRVRHLVVWASAARLARRGSWARERAD
RARFRRRVAEAEAVIGPCLGPEARARALARGAGPA
NSV

Therefore, preferably the RNA construct of the first aspect comprises a nucleotide sequence which encodes an amino acid sequence substantially as set out in SEQ ID No: 6, or a variant or fragment thereof.

In one embodiment, the HSV ICP34.5 polypeptide is encoded by the DNA nucleotide sequence of SEQ ID No: 7, as follows:

[SEQ ID No: 7]
ATGGCCCGCCGCCGCCGCCATCGCGGCCCCCGCCG
CCCCCGGCCGCCCGGGCCCACGGGCGCCGTCCCAA
CCGCACAGTCCCAGGTAACCTCCACGCCCAACTCG
GAACCCGCGGTCAGGAGCGCGCCCGCGGCCGCCCC
GCCGCCGCCCCCCGCCGGTGGGCCCCCGCCTTCTT
GTTCGCTGCTGCTGCGCCAGTGGCTCCACGTTCCC
GAGTCCGCGTCCGACGACGACGATGACGACGACTG
GCCGGACAGCCCCCCGCCCGAGCCGGCGCCAGAGG
CCCGGCCCACCGCCGCCGCCCCCCGGCCCCGGCCC
CCACCGCCCGGCGTGGGCCCGGGGGGCGGGGCTGA
CCCCTCCCACCCCCCCTCGCGCCCCTTCCGCCTTC
CGCCGCGCCTCGCCCTCCGCCTGCGCGTCACCGCG
GAGCACCTGGCGCGCCTGCGCCTGCGACGCGCGGG
CGGGGAGGGGGCGCCGGAGCCCCCCGCGACCCCCG
CGACCCCCGCGACCCCCGCGACCCCCGCGACCCCC
GCGCGGGTGCGCTTCTCGCCCCACGTCCGGGTGCG
CCACCTGGTGGTCTGGGCCTCGGCCGCCCGCCTGG
CGCGCCGCGGCTCGTGGGCCCGCGAGCGGGCCGAC
CGGGCTCGGTTCCGGCGCCGGGTGGCGGAGGCCGA
GGCGGTCATCGGGCCGTGCCTGGGGCCCGAGGCCC
GTGCCCGGGCCCTGGCCCGCGGAGCCGGCCCGGCG
AACTCGGTC

Accordingly, preferably the HSV ICP34.5 polypeptide is encoded by the DNA nucleotide sequence substantially as set out in SEQ ID NO: 7, or a variant or fragment thereof.

Thus, the RNA construct may comprise an RNA nucleotide sequence of SEQ ID No: 8, as follows:

[SEQ ID No: 8]
AUGGCCCGCCGCCGCCGCCAUCGCGGCCCCCGCCG
CCCCCGGCCGCCCGGGCCCACGGGCGCCGUCCCAA
CCGCACAGUCCCAGGUAACCUCCACGCCCAACUCG
GAACCCGCGGUCAGGAGCGCGCCCGCGGCCGCCCC
GCCGCCGCCCCCCGCCGGUGGGCCCCCGCCUUCUU
GUUCGCUGCUGCUGCGCCAGUGGCUCCACGUUCCC
GAGUCCGCGUCCGACGACGACGAUGACGACGACUG
GCCGGACAGCCCCCCGCCCGAGCCGGCGCCAGAGG
CCCGGCCCACCGCCGCCGCCCCCCGGCCCCGGCCC
CCACCGCCCGGCGUGGGCCCGGGGGGCGGGGCUGA
CCCCUCCCACCCCCCCUCGCGCCCCUUCCGCCUUC
CGCCGCGCCUCGCCCUCCGCCUGCGCGUCACCGCG
GAGCACCUGGCGCGCCUGCGCCUGCGACGCGCGGG
CGGGGAGGGGGCGCCGGAGCCCCCCGCGACCCCCG
CGACCCCCGCGACCCCCGCGACCCCCGCGACCCCC
GCGCGGGUGCGCUUCUCGCCCCACGUCCGGGUGCG
CCACCUGGUGGUCUGGGCCUCGGCCGCCCGCCUGG
CGCGCCGCGGCUCGUGGGCCCGCGAGCGGGCCGAC
CGGGCUCGGUUCCGGCGCCGGGUGGCGGAGGCCGA
GGCGGUCAUCGGGCCGUGCCUGGGGCCCGAGGCCC
GUGCCCGGGCCCUGGCCCGCGGAGCCGGCCCGGCG
AACUCGGUC

Furthermore, preferably the RNA construct comprises an RNA nucleotide sequence substantially as set out in SEQ ID No: 8, or a variant or fragment thereof.

The inventors then subjected the protein sequence of SEQ ID No: 6 to codon optimisation for human expression, and one embodiment of the codon optimised nucleic acid (DNA) sequence that includes a start (ATG) and a stop (TGA) codon is provided herein as SEQ ID No: 9, as follows:

[SEQ ID No: 9]
ATGGCCAGAAGAAGGCGGCACAGAGGACCCAGAAG
GCCTAGACCTCCTGGACCAACAGGTGCCGTTCCTA
CCGCTCAGAGCCAAGTGACCAGCACACCCAATTCT
GAACCTGCCGICAGAAGCGCCCCTGCTGCTGCTCC
TCCACCTCCACCAGCTGGCGGACCTCCACCTTCTT
GTTCTCTGCTGCTGAGACAGIGGCTGCACGTGCCA
GAGTCCGCCTCCGACGATGATGACGATGACGACTG
GCCTGACAGCCCTCCTCCAGAACCTGCTCCTGAAG
CCAGACCTACAGCCGCTGCTCCTAGACCTAGACCA
CCACCTCCAGGTGTTGGACCTGGTGGCGGAGCTGA
TCCTTCTCACCCTCCTAGCAGACCCTTCCGGCTTC
CTCCTAGACTGGCCCTGAGACTGAGAGTGACAGCC
GAACACCTGGCCAGACTGAGACTTCGTAGAGCAGG
CGGAGAAGGCGCTCCTGAACCTCCTGCTACACCAG
CCACACCAGCTACTCCCGCAACTCCTGCCACTCCT
GCTAGAGTGCGGTTTAGCCCTCACGTCCGCGTCAG
ACATCIGGTCGTTTGGGCCTCTGCTGCCCGGCTTG
CTAGAAGAGGATCTTGGGCCAGAGAGAGAGCCGAC
GGGCTAGAGCACTTGCTAGAGGIGCCGGACCTGCC
AACAGCGTGTGA

Hence, preferably the RNA construct comprises a sequence substantially as set out in SEQ ID No: 9, or a fragment or variant thereof.

In an embodiment, the RNA sequence corresponding to the codon optimised DNA sequence of SEQ ID No: 9 that includes a start (AUG) and a stop (UGA) codon is provided herein as SEQ ID No: 10, as follows:

[SEQ ID No: 10]
AUGGCCAGAAGAAGGCGGCACAGAGGACCCAGAAG
GCCUAGACCUCCUGGACCAACAGGUGCCGUUCCUA
CCGCUCAGAGCCAAGUGACCAGCACACCCAAUUCU
GAACCUGCCGUCAGAAGCGCCCCUGCUGCUGCUCC
UCCACCUCCACCAGCUGGCGGACCUCCACCUUCUU
GUUCUCUGCUGCUGAGACAGUGGCUGCACGUGCCA
GAGUCCGCCUCCGACGAUGAUGACGAUGACGACUG
GCCUGACAGCCCUCCUCCAGAACCUGCUCCUGAAG
CCAGACCUACAGCCGCUGCUCCUAGACCUAGACCA
CCACCUCCAGGUGUUGGACCUGGUGGCGGAGCUGA
UCCUUCUCACCCUCCUAGCAGACCCUUCCGGCUUC
CUCCUAGACUGGCCCUGAGACUGAGAGUGACAGCC
GAACACCUGGCCAGACUGAGACUUCGUAGAGCAGG
CGGAGAAGGCGCUCCUGAACCUCCUGCUACACCAG
CCACACCAGCUACUCCCGCAACUCCUGCCACUCCU
GCUAGAGUGCGGUUUAGCCCUCACGUCCGCGUCAG
ACAUCUGGUCGUUUGGGCCUCUGCUGCCCGGCUUG
CUAGAAGAGGAUCUUGGGCCAGAGAGAGAGCCGAC
CGGGCUAGAUUUCGGAGAAGAGUGGCCGAAGCCGA
GGCCGUGAUUGGACCUUGUCUUGGCCCUGAAGCUC
GGGCUAGAGCACUUGCUAGAGGUGCCGGACCUGCC
AACAGCGUGUGA

Hence, preferably the RNA construct comprises a sequence substantially as set out in SEQ ID No: 10, or a fragment or variant thereof.

In one embodiment, the at least one IIP may be HCV E2 (hepatitis C virus E2; NS1 Protein from polyprotein ADC54662.1; Accession Number—Genomic RNA Translation ADC54662.1; UniProtKB—D3W8R2 (D3W8R2_9HEPC)), or an orthologue thereof. One action of HCV E2 is to inhibit PKR. Taylor D R, Shi S T, Romano P R, Barber G N, Lai M M C (1999). Inhibition of the interferon-inducible protein kinase PKR by HCV E2 protein. Science, 285, 107-110. doi: 10.1126/science.285.5424.107 One embodiment of the HCV E2 polypeptide sequence is represented herein as SEQ ID No:11, as follows:

[SEQ ID No: 11]
METHVTGGSAGHTVSGFVSLLAPGAKONVQLINTN
GSWHLNSTALNCNDSLNTGWLAGLFYHHKFNSSGC
PERLASCRPLTDFDQGWGPISYANGSGPDQRPYCW
HYPPKPCGIVPAKSVCGPVYCFTPSPVVVGTTDRS
GAPTYSWGENDTDVFVLNNTRPPLGNWFGCTWMNS
TGFTKVCGAPPCVIGGAGNNTLHCPTDCFRKHPDA
TYSRCGSGPWITPRCLVDYPYRLWHYPCTINYTIF
KIRMYVGGVEHRLEAACNWTRGERCDLEDRDRSEL
SPLLLTTTQWQVLPCSFTTLPALSTGLIHLHQNIV
DVQYLYGVGSSIASWAIKWEYVVLLFLLLADARVC
SCLWMMLLISQAEA

Therefore, preferably the RNA construct of the first aspect comprises a nucleotide sequence which encodes an amino acid sequence substantially as set out in SEQ ID No: 11, or a variant or fragment thereof.

In one embodiment, the HCV E2 polypeptide is encoded by the DNA nucleotide sequence of SEQ ID No: 12, as follows:

[SEQ ID No: 12]
ATGGAAACCCACGTCACCGGGGGAAGTGCCGGCCA
CACTGIGTCTGGATTTGTTAGCCTCCTCGCACCAG
GCGCCAAGCAGAACGTCCAGCTGATCAACACCAAC
GGCAGTTGGCACCTCAATAGCACGGCCCTGAACTG
CAATGATAGCCTTAACACCGGCTGGTTAGCAGGGC
TTTTCTATCACCACAAGTTCAACTCTTCAGGCTGT
ACGGAAGCGGCCCCGACCAGCGCCCCTACTGCTGG
CACTACCCCCCTAAACCTTGCGGTGTTGTGCCCGC
GAAGAGTGTGIGTGGTCCGGTATATTGCTTCACTC
CCAGCCCCGTGGTGGTGGGAACGACCGACAGGTCG
GGCGCGCCTACCIACAGCIGGGGTGAAAATGATAC
GGACGTCTTCGTCCTTAACAATACCAGGCCACCGC
TGGGCAATTGGTTCGGTTGTACCTGGATGAACTCA
ACTGGATTCACCAAAGTGTGCGGAGCGCCTCCCTG
TGTCATCGGAGGGGCGGGCAACAACACCCTGCACT
GCCCCACTGATTGCTTCCGCAAGCATCCGGACGCC
ACATACTCTCGGTGCGGCTCCGGTCCCTGGATCAC
ACCCAGGTGCCTGGTCGACTACCCGTATAGGCTTT
GGCATTATCCTTGTACCATCAACTACACCATATTT
AAAATCAGGATGTACGTGGGAGGGGTCGAGCACAG
GCTGGAAGCTGCTTGCAACTGGACGCGGGGCGAAC
GTTGCGATCTGGAAGACAGGGACAGGTCCGAGCTC
AGCCCGTTACTGCTGACCACTACACAGTGGCAGGT
CCTCCCGTGTTCCTTCACAACCCTGCCAGCCTTGT
CCACCGGCCTCATCCACCTCCACCAGAACATTGTG
GACGTGCAGTACTTGTACGGGGTGGGGTCAAGCAT
CGCGTCCTGGGCCATTAAGTGGGAGTACGTCGTTC
TCCTGTTCCTTCTGCTTGCAGACGCGCGCGTCTGC
TCCTGCTTGTGGATGATGCTACTCATATCCCAAGC
GGAGGCG

Accordingly, preferably the HCV E2 polypeptide is encoded by the DNA nucleotide sequence substantially as set out in SEQ ID NO: 12, or a variant or fragment thereof.

Thus, the RNA construct may comprise an RNA nucleotide sequence of SEQ ID No: 13, as follows:

[SEQ ID No: 13]
AUGGAAACCCACGUCACCGGGGGAAGUGCCGGCCA
CACUGUGUCUGGAUUUGUUAGCCUCCUCGCACCAG
GCGCCAAGCAGAACGUCCAGCUGAUCAACACCAAC
GGCAGUUGGCACCUCAAUAGCACGGCCCUGAACUG
CAAUGAUAGCCUUAACACCGGCUGGUUAGCAGGGC
UUUUCUAUCACCACAAGUUCAACUCUUCAGGCUGU
CCUGAGAGGCUAGCCAGCUGCCGACCCCUUACCGA
UUUUGACCAGGGCUGGGGCCCUAUCAGUUAUGCCA
ACGGAAGCGGCCCCGACCAGCGCCCCUACUGCUGG
CACUACCCCCCUAAACCUUGCGGUGUUGUGCCCGC
GAAGAGUGUGUGUGGUCCGGUAUAUUGCUUCACUC
CCAGCCCCGUGGUGGUGGGAACGACCGACAGGUCG
GGCGCGCCUACCUACAGCUGGGGUGAAAAUGAUAC
GGACGUCUUCGUCCUUAACAAUACCAGGCCACCGC
UGGGCAAUUGGUUCGGUUGUACCUGGAUGAACUCA
ACUGGAUUCACCAAAGUGUGCGGAGCGCCUCCCUG
UGUCAUCGGAGGGGCGGGCAACAACACCCUGCACU
GCCCCACUGAUUGCUUCCGCAAGCAUCCGGACGCC
ACAUACUCUCGGUGCGGCUCCGGUCCCUGGAUCAC
ACCCAGGUGCCUGGUCGACUACCCGUAUAGGCUUU
GGCAUUAUCCUUGUACCAUCAACUACACCAUAUUU
AAAAUCAGGAUGUACGUGGGAGGGGUCGAGCACAG
GCUGGAAGCUGCUUGCAACUGGACGCGGGGCGAAC
GUUGCGAUCUGGAAGACAGGGACAGGUCCGAGCUC
AGCCCGUUACUGCUGACCACUACACAGUGGCAGGU
CCUCCCGUGUUCCUUCACAACCCUGCCAGCCUUGU
CCACCGGCCUCAUCCACCUCCACCAGAACAUUGUG
GACGUGCAGUACUUGUACGGGGUGGGGUCAAGCAU
CGCGUCCUGGGCCAUUAAGUGGGAGUACGUCGUUC
UCCUGUUCCUUCUGCUUGCAGACGCGCGCGUCUGC
UCCUGCUUGUGGAUGAUGCUACUCAUAUCCCAAGC
GGAGGCG

Furthermore, preferably the RNA construct comprises an RNA nucleotide sequence substantially as set out in SEQ ID No: 13, or a variant or fragment thereof.

The inventors then subjected the protein sequence of SEQ ID No: 11 to codon optimisation for human expression, and one embodiment of the codon optimised nucleic acid (DNA) sequence that includes a start (ATG) and a stop (TGA) codon is provided herein as SEQ ID No: 14, as follows:

[SEQ ID No: 14]
ATGGAAACCCACGTGACAGGCGGATCTGCCGGCCA
TACAGTGTCCGGCTTTGTGTCTCTTCIGGCCCCTG
GCGCCAAGCAGAATGTGCAGCTGATCAACACCAAC
GGCAGCTGGCACCTGAACAGCACAGCCCTGAACTG
CAACGACAGCCTGAATACCGGATGGCTGGCCGGCC
TGTTCTACCACCACAAGTTCAATAGCAGCGGCTGC
CCCGAGAGACIGGCCTCTTGTAGACCTCTGACCGA
CTTCGATCAAGGCTGGGGCCCTATCAGCTACGCCA
ATGGCTCTGGACCTGACCAGAGGCCTTACTGCTGG
CACTACCCTCCAAAGCCTTGCGGAATCGTGCCTGC
CAAGTCTGTGTGTGGCCCCGTGTACTGCTTCACCC
CATCTCCAGTGGTCGTGGGCACCACCGATAGATCT
GGCGCCCCAACATATAGCIGGGGCGAGAACGACAC
CGACGTGTTCGTGCTGAACAACACCCGGCCTCCAC
TCGGAAATTGGTTCGGCTGCACCTGGATGAACTCC
ACCGGCTTCACAAAAGTGIGCGGAGCCCCTCCTTG
TGTGATTGGCGGAGCCGGAAACAATACCCTGCACT
GCCCTACCGACTGCTTCAGAAAGCACCCCGACGCC
ACCTACAGCAGATGTGGATCTGGCCCTTGGATCAC
CCCTAGATGCCTGGTGGACTACCCCTACCGGCTGT
GGCACTATCCCTGCACCATCAACTACACCATCTTC
AAGATCCGTATGTACGTCGGCGGCGTGGAACACAG
ACTGGAAGCCGCCTGTAACTGGACCAGGGGCGAGA
GATGCGACCTGGAAGATAGAGACAGAAGCGAGCTG
AGCCCTCTGCTGCTGACCACCACACAGTGGCAGGT
CCTGCCTTGCAGCTTCACCACACTGCCTGCTCTGA
GCACCGGCCTGATTCATCTGCACCAGAACATCGTG
GACGTGCAGTACCTGTACGGCGTGGGAAGCTCTAT
TGCCAGCTGGGCCATCAAGTGGGAGTACGTGGIGC
TGCTGTTCCTGCTGCIGGCCGATGCCAGAGIGTGT
AGCTGCCTGTGGATGATGCTGCTGATCTCTCAGGC
CGAGGCCTGA

Hence, preferably the RNA construct is encoded by a DNA sequence substantially as set out in SEQ ID No: 14, or a fragment or variant thereof.

In an embodiment, the RNA sequence corresponding to the codon optimised DNA sequence of SEQ ID No: 14 that includes a start (AUG) and a stop (UGA) codon is provided herein as SEQ ID No: 15, as follows:

[SEQ ID No: 15]
AUGGAAACCCACGUGACAGGCGGAUCUGCCGGCCA
UACAGUGUCCGGCUUUGUGUCUCUUCUGGCCCCUG
GCGCCAAGCAGAAUGUGCAGCUGAUCAACACCAAC
GGCAGCUGGCACCUGAACAGCACAGCCCUGAACUG
CAACGACAGCCUGAAUACCGGAUGGCUGGCCGGCC
UGUUCUACCACCACAAGUUCAAUAGCAGCGGCUGC
CCCGAGAGACUGGCCUCUUGUAGACCUCUGACCGA
CUUCGAUCAAGGCUGGGGCCCUAUCAGCUACGCCA
AUGGCUCUGGACCUGACCAGAGGCCUUACUGCUGG
CACUACCCUCCAAAGCCUUGCGGAAUCGUGCCUGC
CAAGUCUGUGUGUGGCCCCGUGUACUGCUUCACCC
CAUCUCCAGUGGUCGUGGGCACCACCGAUAGAUCU
GGCGCCCCAACAUAUAGCUGGGGCGAGAACGACAC
CGACGUGUUCGUGCUGAACAACACCCGGCCUCCAC
UCGGAAAUUGGUUCGGCUGCACCUGGAUGAACUCC
ACCGGCUUCACAAAAGUGUGCGGAGCCCCUCCUUG
UGUGAUUGGCGGAGCCGGAAACAAUACCCUGCACU
GCCCUACCGACUGCUUCAGAAAGCACCCCGACGCC
ACCUACAGCAGAUGUGGAUCUGGCCCUUGGAUCAC
CCCUAGAUGCCUGGUGGACUACCCCUACCGGCUGU
GGCACUAUCCCUGCACCAUCAACUACACCAUCUUC
AAGAUCCGUAUGUACGUCGGCGGCGUGGAACACAG
ACUGGAAGCCGCCUGUAACUGGACCAGGGGCGAGA
GAUGCGACCUGGAAGAUAGAGACAGAAGCGAGCUG
AGCCCUCUGCUGCUGACCACCACACAGUGGCAGGU
CCUGCCUUGCAGCUUCACCACACUGCCUGCUCUGA
GCACCGGCCUGAUUCAUCUGCACCAGAACAUCGUG
GACGUGCAGUACCUGUACGGCGUGGGAAGCUCUAU
UGCCAGCUGGGCCAUCAAGUGGGAGUACGUGGUGC
UGCUGUUCCUGCUGCUGGCCGAUGCCAGAGUGUGU
AGCUGCCUGUGGAUGAUGCUGCUGAUCUCUCAGGC
CGAGGCCUGA

Hence, preferably the RNA construct comprises a sequence substantially as set out in SEQ ID No: 15, or a fragment or variant thereof.

In one embodiment, the at least one IIP may be HCV NS5a (hepatitis C virus NS5a; isolate H—Genomic RNA translation: AAA45534.1; UniProtKB—P27958 (POLG_HCV77)), or an orthologue thereof. One embodiment of the HCV NS5a polypeptide sequence is represented herein as SEQ ID No:16, as follows:

[SEQ ID No: 16]
MSGSWLRDIWDWICEVLSDFKTWLKAKLMPQLPGI
PFVSCQRGYRGVWRGDGIMHTRCHCGAEITGHVKN
GTMRIVGPRTCKNMWSGTFFINAYTTGPCTPLPAP
NYKFALWRVSAEEYVEIRRVGDFHYVSGMTTDNLK
CPCQIPSPEFFTELDGVRLHRFAPPCKPLLREEVS
FRVGLHEYPVGSQLPCEPEPDVAVLTSMLTDPSHI
TAEAAGRRLARGSPPSMASSSASQLSAPSLKATCT
ANHDSPDAELIEANLLWRQEMGGNITRVESENKVV
ILDSFDPLVAEEDEREVSVPAEILRKSRRFAPALP
VWARPDYNPLLVETWKKPDYEPPVVHGCPLPPPRS
PPVPPPRKKRTVVLTESTLPTALAELATKSFGSSS
TSGITGDNTTTSSEPAPSGCPPDSDVESYSSMPPL
EGEPGDPDLSDGSWSTVSSGADTEDVVCC

Therefore, preferably the RNA construct of the first aspect comprises a nucleotide sequence which encodes an amino acid sequence substantially as set out in SEQ ID No: 16, or a variant or fragment thereof.

In one embodiment, the HCV NS5a polypeptide is encoded by the DNA nucleotide sequence of SEQ ID No: 17, as follows:

[SEQ ID No: 17]
ATGTCCGGTTCCTGGCTAAGGGACATCTGGGACTG
GATATGCGAGGTGCTGAGCGACTTTAAGACCTGGC
TGAAAGCCAAGCTCATGCCACAACTGCCTGGGATT
CCCTTTGTGTCCTGCCAGCGCGGGTATAGGGGGGT
CTGGCGAGGAGACGGCATTATGCACACTCGCTGCC
ACTGTGGAGCTGAGATCACTGGACATGTCAAAAAC
GGGACGATGAGGATCGTCGGTCCTAGGACCTGCAA
GAACATGTGGAGTGGGACGTTCTTCATTAATGCCT
ACACCACGGGCCCCTGTACTCCCCTTCCTGCGCCG
AACTATAAGTTCGCGCTGTGGAGGGIGTCTGCAGA
GGAATACGTGGAGATAAGGCGGGTGGGGGACTTCC
ACTACGTATCGGGCATGACTACTGACAATCTCAAA
TGCCCGTGCCAGATCCCATCGCCCGAATTTTTCAC
AGAATTGGACGGGGTGCGCCTACATAGGTTTGCGC
CCCCTTGCAAGCCCTTGCTGCGGGAGGAGGTATCA
TTCAGAGTAGGACTCCACGAGTACCCGGTGGGGTC
GCAATTACCTTGCGAGCCCGAACCGGACGTAGCCG
TGTTGACGTCCATGCTCACTGATCCCTCCCATATA
ACAGCAGAGGCGGCCGGGAGAAGGTTGGCGAGAGG
GTCACCCCCTTCTATGGCCAGCTCCTCGGCTAGCC
AGCTGTCCGCTCCATCTCTCAAGGCAACTTGCACC
GCCAACCATGACTCCCCTGACGCCGAGCTCATAGA
GGCTAACCTCCTGTGGAGGCAGGAGATGGGCGGCA
ACATCACCAGGGTTGAGTCAGAGAACAAAGTGGTG
ATTCTGGACTCCTTCGATCCGCTTGTGGCAGAGGA
GGATGAGCGGGAGGTCTCCGTACCCGCAGAAATTC
TGCGGAAGTCTCGGAGATTCGCCCCAGCCCTGCCC
GTCTGGGCGCGGCCGGACTACAACCCCCTGCTAGT
AGAGACGTGGAAAAAGCCTGACTACGAACCACCTG
IGGTCCATGGCTGCCCGCTACCACCTCCACGGTCC
CCTCCTGTGCCTCCGCCTCGGAAAAAGCGTACGGT
GGTCCTCACCGAATCAACCCTACCTACTGCCTTGG
CCGAGCTTGCCACCAAAAGTTTTGGCAGCTCCTCA
ACTTCCGGCATTACGGGCGACAATACGACAACATC
CTCTGAGCCCGCCCCTTCIGGCTGCCCCCCCGACT
CCGACGTTGAGTCCTATTCTTCCATGCCCCCCCTG
GAGGGGGAGCCIGGGGATCCGGATCTCAGCGACGG
GTCATGGTCGACGGTCAGTAGTGGGGCCGACACGG
AAGATGTCGTGTGCTGC

Accordingly, preferably the HCV NS5a polypeptide is encoded by the DNA nucleotide sequence substantially as set out in SEQ ID NO: 17, or a variant or fragment thereof.

Thus, the RNA construct may comprise an RNA nucleotide sequence of SEQ ID No: 18, as follows:

[SEQ ID No: 18]
AUGUCCGGUUCCUGGCUAAGGGACAUCUGGGACUGGAUAUGCGAGGUGCU
GAGCGACUUUAAGACCUGGCUGAAAGCCAAGCUCAUGCCACAACUGCCUG
GGAUUCCCUUUGUGUCCUGCCAGCGCGGGUAUAGGGGGGUCUGGCGAGGA
GACGGCAUUAUGCACACUCGCUGCCACUGUGGAGCUGAGAUCACUGGACA
UGUCAAAAACGGGACGAUGAGGAUCGUCGGUCCUAGGACCUGCAAGAACA
UGUGGAGUGGGACGUUCUUCAUUAAUGCCUACACCACGGGCCCCUGUACU
CCCCUUCCUGCGCCGAACUAUAAGUUCGCGCUGUGGAGGGUGUCUGCAGA
GGAAUACGUGGAGAUAAGGCGGGUGGGGGACUUCCACUACGUAUCGGGCA
UGACUACUGACAAUCUCAAAUGCCCGUGCCAGAUCCCAUCGCCCGAAUUU
UUCACAGAAUUGGACGGGGUGCGCCUACAUAGGUUUGCGCCCCCUUGCAA
GCCCUUGCUGCGGGAGGAGGUAUCAUUCAGAGUAGGACUCCACGAGUACC
CGGUGGGGUCGCAAUUACCUUGCGAGCCCGAACCGGACGUAGCCGUGUUG
ACGUCCAUGCUCACUGAUCCCUCCCAUAUAACAGCAGAGGCGGCCGGGAG
AAGGUUGGCGAGAGGGUCACCCCCUUCUAUGGCCAGCUCCUCGGCUAGCC
AGCUGUCCGCUCCAUCUCUCAAGGCAACUUGCACCGCCAACCAUGACUCC
CCUGACGCCGAGCUCAUAGAGGCUAACCUCCUGUGGAGGCAGGAGAUGGG
CGGCAACAUCACCAGGGUUGAGUCAGAGAACAAAGUGGUGAUUCUGGACU
CCUUCGAUCCGCUUGUGGCAGAGGAGGAUGAGCGGGAGGUCUCCGUACCC
GCAGAAAUUCUGCGGAAGUCUCGGAGAUUCGCCCCAGCCCUGCCCGUCUG
GGCGCGGCCGGACUACAACCCCCUGCUAGUAGAGACGUGGAAAAAGCCUG
ACUACGAACCACCUGUGGUCCAUGGCUGCCCGCUACCACCUCCACGGUCC
CCUCCUGUGCCUCCGCCUCGGAAAAAGCGUACGGUGGUCCUCACCGAAUC
AACCCUACCUACUGCCUUGGCCGAGCUUGCCACCAAAAGUUUUGGCAGCU
CCUCAACUUCCGGCAUUACGGGCGACAAUACGACAACAUCCUCUGAGCCC
GCCCCUUCUGGCUGCCCCCCCGACUCCGACGUUGAGUCCUAUUCUUCCAU
GCCCCCCCUGGAGGGGGAGCCUGGGGAUCCGGAUCUCAGCGACGGGUCAU
GGUCGACGGUCAGUAGUGGGGCCGACACGGAAGAUGUCGUGUGCUGC

Furthermore, preferably the RNA construct comprises an RNA nucleotide sequence substantially as set out in SEQ ID No: 18, or a variant or fragment thereof.

The inventors then subjected the protein sequence of SEQ ID No: 16 to codon optimisation for human expression, and one embodiment of the codon optimised nucleic acid (DNA) sequence that includes a start (ATG) and a stop (TGA) codon is provided herein as SEQ ID No: 19, as follows:

[SEQ ID No: 19]
ATGTCTGGCAGCTGGCTGAGAGACATCTGGGACTGGATTTGCGAGGTGCT
GAGCGACTTCAAGACCTGGCTGAAGGCCAAGCTGATGCCTCAGCTGCCTG
GCATCCCTTTCGTGTCCTGTCAGAGGGGCTATAGAGGCGTTTGGAGAGGC
GACGGCATCATGCACACCAGATGTCACTGTGGCGCCGAGATCACAGGCCA
CGTGAAGAACGGCACCATGAGAATCGTGGGCCCCAGAACCTGCAAGAATA
TGTGGAGCGGCACCTTCTTCATCAACGCCTACACCACCGGACCTTGCACA
CCTCTGCCTGCTCCTAACTACAAGTTCGCCCTGTGGCGGGTGTCCGCCGA
GGAATACGTGGAAATCAGAAGAGTGGGCGACTTCCACTACGTGTCCGGCA
TGACCACCGACAACCTGAAGTGCCCCTGTCAGATCCCATCTCCTGAGTTC
TTCACCGAGCTGGATGGCGTGCGGCTGCACAGATTTGCCCCTCCATGTAA
ACCCCTGCTGAGAGAAGAGGTGTCCTTTAGAGTGGGCCTGCACGAGTACC
CTGTGGGTTCTCAGCTCCCTTGCGAGCCTGAACCTGATGTGGCCGTGCTG
ACCTCCATGCTGACAGACCCTTCTCACATCACAGCCGAGGCCGCTGGAAG
AAGGCTGGCTAGAGGATCTCCTCCTAGCATGGCCTCTAGCAGCGCCTCTC
AACTGTCTGCCCCAAGCCTGAAAGCCACCTGTACCGCCAATCACGACAGC
CCTGATGCCGAGCTGATCGAGGCTAACCTGCTGTGGCGGCAAGAGATGGG
CGGCAACATCACCAGAGTGGAAAGCGAGAACAAGGTGGTCATCCTGGATA
GCTTCGACCCTCTGGTGGCCGAAGAGGACGAGAGGGAAGTGTCTGTGCCT
GCCGAGATCCTGAGAAAGAGCAGAAGATTCGCCCCTGCTCTGCCCGTGTG
GGCCAGACCTGATTACAATCCCCTGCTGGTGGAAACATGGAAGAAGCCCG
ACTACGAGCCTCCTGTGGTGCACGGATGTCCACTGCCTCCACCTAGATCT
CCACCTGTGCCACCTCCACGGAAGAAAAGAACCGTGGTGCTGACCGAGAG
CACCCTGCCTACAGCTCTGGCTGAGCTGGCCACAAAGAGCTTTGGCAGCA
GCAGCACCTCTGGCATCACCGGCGATAATACCACCACCAGCTCTGAGCCT
GCTCCAAGCGGATGTCCTCCTGACTCCGACGTGGAAAGCTACAGCAGCAT
GCCTCCTCTGGAAGGCGAACCCGGCGATCCTGATCTGTCTGATGGCTCTT
GGAGCACCGTGTCCTCTGGCGCCGATACAGAGGATGTCGTGTGCTGCTGA

Hence, preferably the RNA construct comprises a sequence substantially as set out in SEQ ID No: 19, or a fragment or variant thereof.

In an embodiment, the RNA sequence corresponding to the codon optimised DNA sequence of SEQ ID No: 19 that includes a start (AUG) and a stop (UGA) codon is provided herein as SEQ ID No: 20, as follows:

[SEQ ID No: 20]
AUGUCUGGCAGCUGGCUGAGAGACAUCUGGGACUGGAUUUGCGAGGUGCU
GAGCGACUUCAAGACCUGGCUGAAGGCCAAGCUGAUGCCUCAGCUGCCUG
GCAUCCCUUUCGUGUCCUGUCAGAGGGGCUAUAGAGGCGUUUGGAGAGGC
GACGGCAUCAUGCACACCAGAUGUCACUGUGGCGCCGAGAUCACAGGCCA
CGUGAAGAACGGCACCAUGAGAAUCGUGGGCCCCAGAACCUGCAAGAAUA
UGUGGAGCGGCACCUUCUUCAUCAACGCCUACACCACCGGACCUUGCACA
CCUCUGCCUGCUCCUAACUACAAGUUCGCCCUGUGGCGGGUGUCCGCCGA
GGAAUACGUGGAAAUCAGAAGAGUGGGCGACUUCCACUACGUGUCCGGCA
UGACCACCGACAACCUGAAGUGCCCCUGUCAGAUCCCAUCUCCUGAGUUC
UUCACCGAGCUGGAUGGCGUGCGGCUGCACAGAUUUGCCCCUCCAUGUAA
ACCCCUGCUGAGAGAAGAGGUGUCCUUUAGAGUGGGCCUGCACGAGUACC
CUGUGGGUUCUCAGCUCCCUUGCGAGCCUGAACCUGAUGUGGCCGUGCUG
ACCUCCAUGCUGACAGACCCUUCUCACAUCACAGCCGAGGCCGCUGGAAG
AAGGCUGGCUAGAGGAUCUCCUCCUAGCAUGGCCUCUAGCAGCGCCUCUC
AACUGUCUGCCCCAAGCCUGAAAGCCACCUGUACCGCCAAUCACGACAGC
CCUGAUGCCGAGCUGAUCGAGGCUAACCUGCUGUGGCGGCAAGAGAUGGG
CGGCAACAUCACCAGAGUGGAAAGCGAGAACAAGGUGGUCAUCCUGGAUA
GCUUCGACCCUCUGGUGGCCGAAGAGGACGAGAGGGAAGUGUCUGUGCCU
GCCGAGAUCCUGAGAAAGAGCAGAAGAUUCGCCCCUGCUCUGCCCGUGUG
GGCCAGACCUGAUUACAAUCCCCUGCUGGUGGAAACAUGGAAGAAGCCCG
ACUACGAGCCUCCUGUGGUGCACGGAUGUCCACUGCCUCCACCUAGAUCU
CCACCUGUGCCACCUCCACGGAAGAAAAGAACCGUGGUGCUGACCGAGAG
CACCCUGCCUACAGCUCUGGCUGAGCUGGCCACAAAGAGCUUUGGCAGCA
GCAGCACCUCUGGCAUCACCGGCGAUAAUACCACCACCAGCUCUGAGCCU
GCUCCAAGCGGAUGUCCUCCUGACUCCGACGUGGAAAGCUACAGCAGCAU
GCCUCCUCUGGAAGGCGAACCCGGCGAUCCUGAUCUGUCUGAUGGCUCUU
GGAGCACCGUGUCCUCUGGCGCCGAUACAGAGGAUGUCGUGUGCUGCUGA

Hence, preferably the RNA construct comprises a sequence substantially as set out in SEQ ID No: 20, or a fragment or variant thereof.

In one embodiment, the at least one IIP may be VACV E3L (vaccinia virus E3L; AEY72868.1; Accession Number—Genomic DNA Translation: AEY72868.1; UniProtKB—H2DSW3 (H2DSW3_9POXV)), or an orthologue thereof. VACV E3L would inhibit innate sensing systems through inhibition of PKR and IRF3 and 7 Perdiuero B, Esteban M (2009) The interferon system and vaccinia virus evasion mechanisms. J Interferon Cytokine Res, 29, 9, 581-198.

One embodiment of the VACV E3L polypeptide sequence is represented herein as SEQ ID No:21, as follows:

[SEQ ID No: 21]
MSKIYIDERSDAEIVCAAIKNIGIEGATAAQLTRQLNMEKREVNKALYDL
QRSAMVYSSDDIPPRWFMTTEADKPDADAMADVIIDDVSREKSMREDHKS
FDDVIPAKKIIDWKDANPVTIINEYCQITKRDWSFRIESVGPSNSPTFYA
CVDIDGRVFDKADGKSKRDAKNNAAKLAVDKLLGYVIIRF

Therefore, preferably the RNA construct of the first aspect comprises a nucleotide sequence which encodes an amino acid sequence substantially as set out in SEQ ID No: 21, or a variant or fragment thereof.

In one embodiment, the VACV E3L polypeptide is encoded by the DNA nucleotide sequence of SEQ ID No: 22, as follows:

[SEQ ID No: 22]
ATGTCTAAGATCTATATCGACGAGCGTTCTGACGCAGAGATTGTGTGTGC
GGCTATTAAAAACATTGGAATCGAAGGAGCTACTGCTGCACAACTAACTA
GACAACTTAATATGGAGAAGCGAGAAGTTAATAAAGCTCTGTACGATCTT
CAACGTAGTGCTATGGTGTACAGCTCCGACGATATTCCTCCTCGTTGGTT
TATGACAACGGAGGCGGATAAGCCGGATGCTGATGCTATGGCTGACGTCA
TAATAGATGATGTATCCCGCGAAAAATCAATGAGAGAGGATCATAAGTCT
TTTGATGATGTTATTCCGGCTAAAAAAATTATTGATTGGAAAGATGCTAA
CCCTGTCACCATTATTAATGAGTACTGCCAAATAACTAAGAGAGATTGGT
CTTTTCGTATTGAATCAGTTGGGCCTAGTAACTCTCCTACATTTTATGCC
TGTGTAGATATCGACGGAAGAGTATTCGATAAGGCCGATGGAAAATCTAA
ACGAGATGCTAAAAATAATGCAGCTAAATTGGCTGTAGATAAACTTCTTG
GGTACGTCATCATTAGATTC

Accordingly, preferably the VACV E3L polypeptide is encoded by the DNA nucleotide sequence substantially as set out in SEQ ID NO: 22, or a variant or fragment thereof.

Thus, the RNA construct may comprise an RNA nucleotide sequence of SEQ ID No: 23, as follows:

[SEQ ID No: 23]
AUGUCUAAGAUCUAUAUCGACGAGCGUUCUGACGCAGAGAUUGUGUGUGC
GGCUAUUAAAAACAUUGGAAUCGAAGGAGCUACUGCUGCACAACUAACUA
GACAACUUAAUAUGGAGAAGCGAGAAGUUAAUAAAGCUCUGUACGAUCUU
CAACGUAGUGCUAUGGUGUACAGCUCCGACGAUAUUCCUCCUCGUUGGUU
UAUGACAACGGAGGCGGAUAAGCCGGAUGCUGAUGCUAUGGCUGACGUCA
UAAUAGAUGAUGUAUCCCGCGAAAAAUCAAUGAGAGAGGAUCAUAAGUCU
UUUGAUGAUGUUAUUCCGGCUAAAAAAAUUAUUGAUUGGAAAGAUGCUAA
CCCUGUCACCAUUAUUAAUGAGUACUGCCAAAUAACUAAGAGAGAUUGGU
CUUUUCGUAUUGAAUCAGUUGGGCCUAGUAACUCUCCUACAUUUUAUGCC
UGUGUAGAUAUCGACGGAAGAGUAUUCGAUAAGGCCGAUGGAAAAUCUAA
ACGAGAUGCUAAAAAUAAUGCAGCUAAAUUGGCUGUAGAUAAACUUCUUG
GGUACGUCAUCAUUAGAUUC

Furthermore, preferably the RNA construct comprises an RNA nucleotide sequence substantially as set out in SEQ ID No: 23, or a variant or fragment thereof.

The inventors then subjected the protein sequence of SEQ ID No: 21 to codon optimisation for human expression, and one embodiment of the codon optimised nucleic acid (DNA) sequence that includes a start (ATG) and a stop (TGA) codon is provided herein as SEQ ID No: 24, as follows:

[SEQ ID No: 24]
ATGAGCAAGATCTACATCGACGAGCGGAGCGACGCCGAGATTGTGTGTGC
CGCCATCAAGAACATCGGCATCGAAGGCGCTACAGCCGCTCAGCTGACCA
GACAGCTGAACATGGAAAAGCGGGAAGTGAACAAGGCCCTGTACGACCTG
CAGAGAAGCGCCATGGTGTACAGCAGCGACGACATCCCTCCTCGGTGGTT
TATGACCACAGAGGCCGACAAGCCCGACGCCGATGCTATGGCCGATGTGA
TCATCGACGACGTGTCCCGCGAGAAGTCCATGAGAGAGGACCACAAGAGC
TTCGATGACGTGATCCCCGCCAAGAAGATCATCGATTGGAAGGACGCCAA
TCCTGTGACCATCATCAACGAGTACTGCCAGATCACCAAGCGCGACTGGT
CCTTCAGAATCGAGAGCGTGGGCCCCAGCAACAGCCCTACCTTTTATGCC
TGCGTGGACATCGACGGCCGGGTGTTCGATAAGGCCGATGGCAAGAGCAA
GCGGGACGCCAAAAACAACGCCGCCAAACTGGCCGTGGATAAGCTGCTGG
GCTACGTGATCATCCGGTTCTGA

Hence, preferably the RNA construct is encoded by a DNA sequence substantially as set out in SEQ ID No: 24, or a fragment or variant thereof.

In an embodiment, the RNA sequence corresponding to the codon optimised DNA sequence of SEQ ID No: 24 that includes a start (AUG) and a stop (UGA) codon is provided herein as SEQ ID No: 25, as follows:

[SEQ ID No: 25]
AUGAGCAAGAUCUACAUCGACGAGCGGAGCGACGCCGAGAUUGUGUGUGC
CGCCAUCAAGAACAUCGGCAUCGAAGGCGCUACAGCCGCUCAGCUGACCA
GACAGCUGAACAUGGAAAAGCGGGAAGUGAACAAGGCCCUGUACGACCUG
CAGAGAAGCGCCAUGGUGUACAGCAGCGACGACAUCCCUCCUCGGUGGUU
UAUGACCACAGAGGCCGACAAGCCCGACGCCGAUGCUAUGGCCGAUGUGA
UCAUCGACGACGUGUCCCGCGAGAAGUCCAUGAGAGAGGACCACAAGAGC
UUCGAUGACGUGAUCCCCGCCAAGAAGAUCAUCGAUUGGAAGGACGCCAA
UCCUGUGACCAUCAUCAACGAGUACUGCCAGAUCACCAAGCGCGACUGGU
CCUUCAGAAUCGAGAGCGUGGGCCCCAGCAACAGCCCUACCUUUUAUGCC
UGCGUGGACAUCGACGGCCGGGUGUUCGAUAAGGCCGAUGGCAAGAGCAA
GCGGGACGCCAAAAACAACGCCGCCAAACUGGCCGUGGAUAAGCUGCUGG
GCUACGUGAUCAUCCGGUUCUGA

Hence, preferably the RNA construct comprises a sequence substantially as set out in SEQ ID No: 25, or a fragment or variant thereof.

In one embodiment, the at least one IIP may be VACV K3L (vaccinia virus K3L; P20639.1; Accession Number—Genomic DNA Translation: AAA48009.1; UniProtKB—P20639 (K3_VACCC)), or an orthologue thereof. VACV K3L inhibits PKR. Perdiuero B, Esteban M (2009) The interferon system and vaccinia virus evasion mechanisms. J Interferon Cytokine Res, 29, 9, 581-198. One embodiment of the VACV K3L polypeptide sequence is represented herein as SEQ ID No:26, as follows:

[SEQ ID No: 26]
MLAFCYSLPNAGDVIKGRVYEKDYALYIYLFDYPHSEAILAESVKMHMDR
YVEYRDKLVGKTVKVKVIRVDYTKGYIDVNYKRMCRHQ

Therefore, preferably the RNA construct of the first aspect comprises a nucleotide sequence which encodes an amino acid sequence substantially as set out in SEQ ID No: 26, or a variant or fragment thereof.

In one embodiment, the VACV K3L polypeptide is encoded by the DNA nucleotide sequence of SEQ ID No: 27, as follows:

[SEQ ID No: 27]
ATGCTTGCATTTTGTTATTCGTTGCCCAATGCGGGCGATGTAATAAAGGG
CAGAGTATACGAGAAGGATTATGCTCTATACATTTATCTTTTTGACTATC
CTCACTCTGAAGCTATCTTGGCAGAGAGTGTTAAGATGCATATGGATAGA
TATGTTGAATATAGGGATAAACTGGTAGGGAAAACTGTAAAAGTTAAAGT
GATTAGAGTTGATTATACAAAAGGATATATAGATGTCAATTACAAAAGGA
TGTGTAGACATCAA

Accordingly, preferably the VACV K3L polypeptide is encoded by the DNA nucleotide sequence substantially as set out in SEQ ID NO: 27, or a variant or fragment thereof.

Thus, the RNA construct may comprise an RNA nucleotide sequence of SEQ ID No: 28, as follows:

[SEQ ID No: 28]
AUGCUUGCAUUUUGUUAUUCGUUGCCCAAUGCGGGCGAUGUAAUAAAGGG
CAGAGUAUACGAGAAGGAUUAUGCUCUAUACAUUUAUCUUUUUGACUAUC
CUCACUCUGAAGCUAUCUUGGCAGAGAGUGUUAAGAUGCAUAUGGAUAGA
UAUGUUGAAUAUAGGGAUAAACUGGUAGGGAAAACUGUAAAAGUUAAAGU
GAUUAGAGUUGAUUAUACAAAAGGAUAUAUAGAUGUCAAUUACAAAAGGA
UGUGUAGACAUCAA

Furthermore, preferably the RNA construct comprises an RNA nucleotide sequence substantially as set out in SEQ ID No: 28, or a variant or fragment thereof.

The inventors then subjected the protein sequence of SEQ ID No: 26 to codon optimisation for human expression, and one embodiment of the codon optimised nucleic acid (DNA) sequence that includes a start (ATG) and a stop (TGA) codon is provided herein as SEQ ID No: 29, as follows:

[SEQ ID No: 29]
ATGCTGGCCTTCTGCTACAGCCTGCCTAATGCCGGCGACGTGATCAAGGG
CAGAGTGTACGAGAAGGACTACGCCCTGTACATCTACCTGTTCGACTACC
CTCACAGCGAGGCCATCCTGGCCGAGTCTGTGAAGATGCACATGGACAGA
TACGTGGAATACCGGGACAAGCTCGTGGGCAAGACCGTGAAAGTGAAAGT
CATCAGAGTGGACTACACCAAGGGCTACATCGACGTGAACTACAAGCGGA
TGTGCAGGCACCAGTGA

Hence, preferably the RNA construct is encoded by a DNA sequence substantially as set out in SEQ ID No: 29, or a fragment or variant thereof.

In an embodiment, the RNA sequence corresponding to the codon optimised DNA sequence of SEQ ID No: 29 that includes a start (AUG) and a stop (UGA) codon is provided herein as SEQ ID No: 30, as follows:

[SEQ ID No: 30]
AUGCUGGCCUUCUGCUACAGCCUGCCUAAUGCCGGCGACGUGAUCAAGGG
CAGAGUGUACGAGAAGGACUACGCCCUGUACAUCUACCUGUUCGACUACC
CUCACAGCGAGGCCAUCCUGGCCGAGUCUGUGAAGAUGCACAUGGACAGA
UACGUGGAAUACCGGGACAAGCUCGUGGGCAAGACCGUGAAAGUGAAAGU
CAUCAGAGUGGACUACACCAAGGGCUACAUCGACGUGAACUACAAGCGGA
UGUGCAGGCACCAGUGA

Hence, preferably the RNA construct comprises a sequence substantially as set out in SEQ ID No: 30, or a fragment or variant thereof.

In one embodiment, the at least one IIP may be Vaccinia C6 (VACV C6—vaccinia virus C6; Accession Number—Genomic DNA Translation: AAA69602.1; UniProtKB—P17362 (C6_VACCW)), or an orthologue thereof. This protein is believed to prevent the establishment of a cellular antiviral state by blocking virus-induced phosphorylation and activation of interferon regulatory factors 3/IRF3 and 7/IRF7, transcription factors critical for the induction of interferons alpha and beta. This blockage may be produced through the inhibition of host TBK1, by binding host TBK1 adapter proteins TBKBP1 and AZI2, thereby producing a strong inhibition of the phosphorylation and activation of IRF3 and IRF7. It may also act as an inhibitor of the cellular response to type I IFN by interacting with host STAT2. Mechanistically, it may further exert its inhibitory effect after host ISGF3 complex (composed of STAT1. STAT2 and IRF9) binding to the interferon stimulated response element (Smith G L. Vaccinia Virus Protein C6: A Multifunctional Interferon Antagonist. Adv Exp Med Biol. 2018; 1052:1-7. doi: 10.1007/978-981-10-7572-8_1. PMID: 29785476.). One embodiment of the Vaccinia C6 polypeptide sequence is represented herein as SEQ ID No:31, as follows:

[SEQ ID No: 31]
MNAYNKADSFSLESDSIKDVIHDYICWLSMTDEMRPSIGNVFKAMETFKI
DAVRYYDGNIYELAKDINAMSFDGFIRSLQTIASKKDKLTVYGTMGLLSI
VVDINKGCDISNIKFAAGIIILMEYIFDDTDMSHLKVALYRRIQRRDDVD
R

Therefore, preferably the RNA construct of the first aspect comprises a nucleotide sequence which encodes an amino acid sequence substantially as set out in SEQ ID No: 31, or a variant or fragment thereof.

In one embodiment, the Vaccinia C6 polypeptide is encoded by the DNA nucleotide sequence of SEQ ID No: 32, as follows:

[SEQ ID No: 32]
ATGAATGCGTATAATAAAGCCGATTCGTTTTCTTTAGAGTCTGATTCTAT
CAAAGATGTTATACACGATTATATTTGTTGGCTCAGTATGACTGATGAAA
TGAGACCATCTATCGGAAACGTCTTTAAAGCGATGGAAACGTTTAAGATA
GACGCGGTTAGATATTACGATGGTAACATATATGAATTAGCTAAAGATAT
AAATGCGATGTCGTTTGACGGTTTTATAAGATCTCTACAAACTATCGCTT
CAAAGAAAGATAAACTCACTGTTTATGGAACCATGGGACTGCTGTCTATT
GTCGTAGATATTAACAAAGGTTGTGATATATCCAATATCAAGTTCGCTGC
CGGAATAATCATTTTAATGGAGTATATTTTTGATGACACGGATATGTCTC
ATCTTAAAGTAGCACTCTATCGTAGAATACAGAGACGTGATGATGTAGAT
AGA

Accordingly, preferably the Vaccinia C6 polypeptide is encoded by the DNA nucleotide sequence substantially as set out in SEQ ID NO: 32, or a variant or fragment thereof.

Thus, the RNA construct may comprise an RNA nucleotide sequence of SEQ ID No: 33, as follows:

[SEQ ID No: 33]
AUGAAUGCGUAUAAUAAAGCCGAUUCGUUUUCUUUAGAGUCUGAUUCUAU
CAAAGAUGUUAUACACGAUUAUAUUUGUUGGCUCAGUAUGACUGAUGAAA
UGAGACCAUCUAUCGGAAACGUCUUUAAAGCGAUGGAAACGUUUAAGAUA
GACGCGGUUAGAUAUUACGAUGGUAACAUAUAUGAAUUAGCUAAAGAUAU
AAAUGCGAUGUCGUUUGACGGUUUUAUAAGAUCUCUACAAACUAUCGCUU
CAAAGAAAGAUAAACUCACUGUUUAUGGAACCAUGGGACUGCUGUCUAUU
GUCGUAGAUAUUAACAAAGGUUGUGAUAUAUCCAAUAUCAAGUUCGCUGC
CGGAAUAAUCAUUUUAAUGGAGUAUAUUUUUGAUGACACGGAUAUGUCUC
AUCUUAAAGUAGCACUCUAUCGUAGAAUACAGAGACGUGAUGAUGUAGAU
AGA

Furthermore, preferably the RNA construct comprises an RNA nucleotide sequence substantially as set out in SEQ ID No: 33, or a variant or fragment thereof.

The inventors then subjected the protein sequence of SEQ ID No: 31 to codon optimisation for human expression, and one embodiment of the codon optimised nucleic acid (DNA) sequence that includes a start (ATG) and a stop (TGA) codon is provided herein as SEQ ID No: 34, as follows:

[SEQ ID No: 34]
ATGAACGCCTACAACAAGGCCGACAGCTTCAGCCTGGAAAGCGACAGCAT
CAAGGACGTGATCCACGACTACATCTGCTGGCTGAGCATGACCGACGAGA
TGAGGCCCAGCATCGGCAACGTGTTCAAGGCCATGGAAACCTTCAAGATC
GACGCCGTGCGGTACTACGACGGCAACATCTATGAGCTGGCCAAGGACAT
CAACGCCATGAGCTTCGACGGCTTCATCAGAAGCCTGCAGACAATCGCCA
GCAAGAAAGACAAGCTGACCGTGTACGGCACCATGGGCCTGCTGTCTATC
GTGGTGGATATCAACAAGGGCTGCGACATCAGCAACATCAAGTTCGCCGC
TGGCATCATCATCCTGATGGAGTACATCTTCGACGACACCGACATGAGCC
ACCTGAAGGTGGCCCTGTACAGAAGAATCCAGCGGAGGGACGACGTGGAC
AGATGA

Hence, preferably the RNA construct is encoded by a DNA sequence substantially as set out in SEQ ID No: 34, or a fragment or variant thereof.

In an embodiment, the RNA sequence corresponding to the codon optimised DNA sequence of SEQ ID No: 34 that includes a start (AUG) and a stop (UGA) codon is provided herein as SEQ ID No: 35, as follows:

[SEQ ID No: 35]
AUGAACGCCUACAACAAGGCCGACAGCUUCAGCCUGGAAAGCGACAGCAU
CAAGGACGUGAUCCACGACUACAUCUGCUGGCUGAGCAUGACCGACGAGA
UGAGGCCCAGCAUCGGCAACGUGUUCAAGGCCAUGGAAACCUUCAAGAUC
GACGCCGUGCGGUACUACGACGGCAACAUCUAUGAGCUGGCCAAGGACAU
CAACGCCAUGAGCUUCGACGGCUUCAUCAGAAGCCUGCAGACAAUCGCCA
GCAAGAAAGACAAGCUGACCGUGUACGGCACCAUGGGCCUGCUGUCUAUC
GUGGUGGAUAUCAACAAGGGCUGCGACAUCAGCAACAUCAAGUUCGCCGC
UGGCAUCAUCAUCCUGAUGGAGUACAUCUUCGACGACACCGACAUGAGCC
ACCUGAAGGUGGCCCUGUACAGAAGAAUCCAGCGGAGGGACGACGUGGAC
AGAUGA

Hence, preferably the RNA construct comprises a sequence substantially as set out in SEQ ID No: 35, or a fragment or variant thereof.

In one embodiment, the at least one IIP may be MERS ORF8b (Middle East Respiratory Syndrome virus ORF8b; Accession Number—GenBank: ANF29170.1; UniProtKB—A0A1W5LGP6 (A0A1W5LGP6_MERS)), or an orthologue thereof. MERS ORF8b is believed to be an antagonist of MDA5-mediated NF-κB activation and IRF3 activation. (Lee J-Y, Bae S, Myoung J (2019) Middle East Respiratory Syndrome Coronavirus-Encoded Accessory Proteins Impair MDA5- and TBK1-Mediated Activation of NF-κB. J Microbiol Biotechnol, 29, 8,1316-1323 doi: 10.4014/jmb.1908.08004. Wong, L-yR, Ye Z W, Lui P-Y, Zheng X, Yuan S, Zhu L, Fung S-Y et al. (2020) Middle East respiratory syndrome coronavirus ORF8b accessory protein suppresses type I IFN expression by impeding HSP70-dependent activation of IRF3 kinase IKK€. J Immunol, 205, 6, 1564-1579).

One embodiment of the MERS ORF8b polypeptide sequence is represented herein as SEQ ID No:36, as follows:

[SEQ ID No: 36]
MPIPPLRKMLGIGGDRTEKLIPGMELSNWLPGGTSTTLELDPKQHSHSGL
LRMASFGSMKMAPLMLLQLLGRGTLTMIQLLLHNSRPVLSFLKTSTLRGL
EAIVNHLQEPLAQAETLPDLVHKVQDQETLPAALLQVHLESEQ

Therefore, preferably the RNA construct of the first aspect comprises a nucleotide sequence which encodes an amino acid sequence substantially as set out in SEQ ID No: 36, or a variant or fragment thereof.

In one embodiment, the MERS ORF8b polypeptide is encoded by the DNA nucleotide sequence of SEQ ID No: 37, as follows:

[SEQ ID No: 37]
ATGCCAATTCCACCCCTGCGCAAAATGCTGGGTATTGGCGGAGACAGGAC
AGAAAAATTAATACCGGGAATTCCGGGCTGTTAAGGATGGCATCGTTTGG
GTCCATGAAGATGGCGCCACTGATGCTCCTTCAACTTTTGGGACGCGGAA
CCCTAACAATGATTCAGCTATTGTTACACAATTCGCGCCCGGTACTAAGC
TTCCTAAAAACTTCCACATTGAGGGGACTGGAGGCAATAGTCAATCATCT
TCAAGAGCCTCTAGCGCAAGCAGAAACTCTTCCAGATCTAGTTCACAAGG
TTCAAGATCAGGAAACTCTACCCGCGGCACTTCTCCAGGTCCATCTGGAA
TCGGAGCAG

Accordingly, preferably the MERS ORF8b polypeptide is encoded by the DNA nucleotide sequence substantially as set out in SEQ ID NO: 37, or a variant or fragment thereof.

Thus, the RNA construct may comprise an RNA nucleotide sequence of SEQ ID No: 38, as follows:

[SEQ ID No: 38]
AUGCCAAUUCCACCCCUGCGCAAAAUGCUGGGUAUUGGCGGAGACAGGAC
AGAAAAAUUAAUACCGGGAAUGGAAUUAAGCAACUGGCUCCCAGGUGGUA
CUUCUACUACACUGGAACUGGACCCGAAGCAGCACUCCCAUUCCGGGCUG
UUAAGGAUGGCAUCGUUUGGGUCCAUGAAGAUGGCGCCACUGAUGCUCCU
UCAACUUUUGGGACGCGGAACCCUAACAAUGAUUCAGCUAUUGUUACACA
AUUCGCGCCCGGUACUAAGCUUCCUAAAAACUUCCACAUUGAGGGGACUG
GAGGCAAUAGUCAAUCAUCUUCAAGAGCCUCUAGCGCAAGCAGAAACUCU
UCCAGAUCUAGUUCACAAGGUUCAAGAUCAGGAAACUCUACCCGCGGCAC
UUCUCCAGGUCCAUCUGGAAUCGGAGCAG

Furthermore, preferably the RNA construct comprises an RNA nucleotide sequence substantially as set out in SEQ ID No: 38, or a variant or fragment thereof.

The inventors then subjected the protein sequence of SEQ ID No: 36 to codon optimisation for human expression, and one embodiment of the codon optimised nucleic acid (DNA) sequence that includes a start (ATG) and a stop (TGA) codon is provided herein as SEQ ID No: 39, as follows:

[SEQ ID No: 39]
ATGCCCATTCCTCCACTGAGAAAGATGCTCGGCATCGGCGGCGACAGAAC
CGAGAAGCTGATCCCTGGCATGGAACTGAGCAACTGGCTGCCTGGCGGCA
CCAGCACAACACTGGAACTGGATCCTAAGCAGCACAGCCACAGCGGCCTG
CTGAGAATGGCCAGCTTTGGCAGCATGAAGATGGCCCCTCTGATGCTGCT
GCAGCTGCTCGGAAGAGGCACCCTGACAATGATCCAGCTGCTGCTCCACA
ACAGCAGACCCGTGCTGAGCTTCCTGAAAACCAGCACACTGAGAGGCCTG
GAAGCCATCGTGAACCATCTGCAAGAGCCCCTGGCTCAGGCCGAGACACT
GCCTGATCTGGTGCACAAGGIGCAGGACCAAGAAACCCTGCCTGCCGCTC
TGCTGCAGGTCCACCTGGAATCTGAGCAGTGA

Hence, preferably the RNA construct is encoded by a DNA sequence substantially as set out in SEQ ID No: 39, or a fragment or variant thereof.

In an embodiment, the RNA sequence corresponding to the codon optimised DNA sequence of SEQ ID No: 39 that includes a start (AUG) and a stop (UGA) codon is provided herein as SEQ ID No: 40, as follows:

[SEQ ID No: 40]
AUGCCCAUUCCUCCACUGAGAAAGAUGCUCGGCAUCGGCGGCGACAGAAC
CGAGAAGCUGAUCCCUGGCAUGGAACUGAGCAACUGGCUGCCUGGCGGCA
CCAGCACAACACUGGAACUGGAUCCUAAGCAGCACAGCCACAGCGGCCUG
CUGAGAAUGGCCAGCUUUGGCAGCAUGAAGAUGGCCCCUCUGAUGCUGCU
GCAGCUGCUCGGAAGAGGCACCCUGACAAUGAUCCAGCUGCUGCUCCACA
ACAGCAGACCCGUGCUGAGCUUCCUGAAAACCAGCACACUGAGAGGCCUG
GAAGCCAUCGUGAACCAUCUGCAAGAGCCCCUGGCUCAGGCCGAGACACU
GCCUGAUCUGGUGCACAAGGUGCAGGACCAAGAAACCCUGCCUGCCGCUC
UGCUGCAGGUCCACCUGGAAUCUGAGCAGUGA

Hence, preferably the RNA construct comprises a sequence substantially as set out in SEQ ID No: 40, or a fragment or variant thereof.

In one embodiment, the at least one IIP may be KSHV ORF52 (Kaposi's sarcoma-associated herpesvirus ORF52; Accession Number—Genomic DNA Translation: ACY00451.1; UniProtKB—F5HBL8 (F5HBL8_HHV8)), or an orthologue thereof. ORF52 protein of Kaposi's sarcoma-associated herpesvirus (KSHV) is believed to directly inhibit cGAS enzymatic activity and thus prevent generation of the signalling molecule cGAMP by binding to both cGAS and DNA. (Wu J-J, Li W, Shao Y, Avey D et al. (2015) Inhibition of cGAS DNA Sensing by a Herpesvirus Virion Protein. Cell Host Microbe. 18, 3, 333-344. doi: 10.1016/j.chom.2015.07.015.).

One embodiment of the KSHV ORF52 polypeptide sequence is represented herein as SEQ ID No:41, as follows:

[SEQ ID No: 41]
MAAPRGRPKKDLTMEDLTAKISQLTVENRELRKALGSTADPRDRPLTATE
KEAQLTATVGALSAAAAKKIEARVRTIFSKVVTQKQVDDALKGLSLRIDV
CMSDGGTAKPPPGANNRRRRGASTTRAGVDD

Therefore, preferably the RNA construct of the first aspect comprises a nucleotide sequence which encodes an amino acid sequence substantially as set out in SEQ ID No: 41, or a variant or fragment thereof.

In one embodiment, the KSHV ORF52 polypeptide is encoded by the DNA nucleotide sequence of SEQ ID No: 42, as follows:

[SEQ ID No: 42]
ATGGCCGCGCCCAGGGGCAGACCCAAAAAGGACCTTACGATGGAAGACCT
AACCGCAAAGATAAGCCAATTGACTGTGGAGAATCGGGAGCTTCGGAAAG
CACTGGGATCCACTGCCGATCCGAGAGATCGGCCTCTGACGGCCACCGAG
AAGGAAGCGCAGCTTACCGCTACTGTGGGTGCACTGAGTGCGGCGGCCGC
TAAGAAAATCGAAGCCAGGGTCAGGACAATATTCAGCAAGGTTGTAACAC
AAAAGCAAGTGGACGATGCCCTGAAGGGACTTTCGCTTAGAATCGACGTC
TGCATGTCAGACGGGGGCACCGCTAAACCGCCTCCTGGTGCCAATAACAG
GCGACGAAGAGGAGCCTCGACAACACGGGCGGGGGTTGATGAC

Accordingly, preferably the KSHV ORF52 polypeptide is encoded by the DNA nucleotide sequence substantially as set out in SEQ ID NO: 42, or a variant or fragment thereof.

Thus, the RNA construct may comprise an RNA nucleotide sequence of SEQ ID No: 43, as follows:

[SEQ ID No: 43]
AUGGCCGCGCCCAGGGGCAGACCCAAAAAGGACCUUACGAUGGAAGACCU
AACCGCAAAGAUAAGCCAAUUGACUGUGGAGAAUCGGGAGCUUCGGAAAG
CACUGGGAUCCACUGCCGAUCCGAGAGAUCGGCCUCUGACGGCCACCGAG
AAGGAAGCGCAGCUUACCGCUACUGUGGGUGCACUGAGUGCGGCGGCCGC
UAAGAAAAUCGAAGCCAGGGUCAGGACAAUAUUCAGCAAGGUUGUAACAC
AAAAGCAAGUGGACGAUGCCCUGAAGGGACUUUCGCUUAGAAUCGACGUC
UGCAUGUCAGACGGGGGCACCGCUAAACCGCCUCCUGGUGCCAAUAACAG
GCGACGAAGAGGAGCCUCGACAACACGGGCGGGGGUUGAUGAC

Furthermore, preferably the RNA construct comprises an RNA nucleotide sequence substantially as set out in SEQ ID No: 43, or a variant or fragment thereof.

The inventors then subjected the protein sequence of SEQ ID No: 41 to codon optimisation for human expression, and one embodiment of the codon optimised nucleic acid (DNA) sequence that includes a start (ATG) and a stop (TGA) codon is provided herein as SEQ ID No: 44, as follows:

[SEQ ID No: 44]
ATGGCTGCTCCTAGAGGCAGACCCAAGAAAGACCTGACCATGGAAGATCT
GACCGCCAAGATCAGCCAGCTGACCGTGGAAAACAGAGAGCTGAGAAAGG
CCCTGGGCAGCACCGCCGATCCTAGAGATAGACCTCTGACAGCCACCGAG
AAAGAGGCCCAGCTGACAGCTACAGTGGGAGCCCTTTCTGCCGCCGCTGC
CAAGAAAATTGAAGCCAGAGTGCGGACCATCTTCAGCAAGGTGGTCACCC
AGAAACAGGTGGACGATGCCCTGAAGGGCCTGAGCCTGAGAATCGACGTG
TGTATGTCTGACGGCGGCACCGCCAAACCTCCACCTGGCGCTAACAACAG
AAGAAGAAGAGGCGCCAGCACCACCAGAGCTGGCGTGGACGATTGA

Hence, preferably the RNA construct is encoded by a DNA sequence substantially as set out in SEQ ID No: 44, or a fragment or variant thereof.

In an embodiment, the RNA sequence corresponding to the codon optimised DNA sequence of SEQ ID No: 44 that includes a start (AUG) and a stop (UGA) codon is provided herein as SEQ ID No: 45, as follows:

[SEQ ID No: 45]
AUGGCUGCUCCUAGAGGCAGACCCAAGAAAGACCUGACCAUGGAAGAUCU
GACCGCCAAGAUCAGCCAGCUGACCGUGGAAAACAGAGAGCUGAGAAAGG
CCCUGGGCAGCACCGCCGAUCCUAGAGAUAGACCUCUGACAGCCACCGAG
AAAGAGGCCCAGCUGACAGCUACAGUGGGAGCCCUUUCUGCCGCCGCUGC
CAAGAAAAUUGAAGCCAGAGUGCGGACCAUCUUCAGCAAGGUGGUCACCC
AGAAACAGGUGGACGAUGCCCUGAAGGGCCUGAGCCUGAGAAUCGACGUG
UGUAUGUCUGACGGCGGCACCGCCAAACCUCCACCUGGCGCUAACAACAG
AAGAAGAAGAGGCGCCAGCACCACCAGAGCUGGCGUGGACGAUUGA

Hence, preferably the RNA construct comprises a sequence substantially as set out in SEQ ID No: 45, or a fragment or variant thereof.

In one embodiment, the at least one IIP may be Ebola VP35 (EBOV VP35; NP_066244.1; Accession Number—NCBI Reference Sequence: NC_002549.1; UniProtKB—Q05127 (VP35_EBOZM)), or an orthologue thereof. Ebola virus VP35 protein is thought to (amongst other effects on innate signalling cascades) bind to the cellular protein PACT, a cellular dsRNA binding protein required for activation of RIG-I, inhibit IRF3 and IRF7 activity (Luthra P, Raman P, Mire C E, Weisand C, Isuda Y et al. (2013) Mutual antagonism between Ebola virus VP35 protein and the RIG-I activator PACT determines infection outcome. Cell Host Microbe., 14(1):74-84. doi: 10.1016/j.chom.2013.06.010. Hartman A L, Bird B H, Towner J S, Anoniadou Z-A, Zaki S R, Nichol S T (2008) Inhibition of IRF-3 activation by VP35 is critical for the high level of virulence of Ebola virus. J Virol. 82, 6, 2699-2704. Audet J, Kobinger G P (2015). Immune evasion in ebolavirus infections. Viral Immunol., 28, 1, 10-18.).

One embodiment of the Ebola VP35 polypeptide sequence is represented herein as SEQ ID No:46, as follows:

[SEQ ID No: 46]
MTTRTKGRGHTAATTQNDRMPGPELSGWISEQLMTGRIPVSDIFCDIENN
PGLCYASQMQQTKPNPKTRNSQTQTDPICNHSFEEVVQTLASLATVVQQQ
TIASESLEQRITSLENGLKPVYDMAKTISSLNRVCAEMVAKYDLLVMTTG
RATATAAATEAYWAEHGQPPPGPSLYEESAIRGKIESRDETVPQSVREAF
NNLNSTTSLTEENFGKPDISAKDLRNIMYDHLPGFGTAFHQLVQVICKLG
KDSNSLDIIHAEFQASLAEGDSPQCALIQITKRVPIFQDAAPPVIHIRSR
GDIPRACQKSLRPVPPSPKIDRGWVCVFQLQDGKTLGLKI

Therefore, preferably the RNA construct of the first aspect comprises a nucleotide sequence which encodes an amino acid sequence substantially as set out in SEQ ID No: 46, or a variant or fragment thereof.

In one embodiment, the Ebola VP35 polypeptide is encoded by the DNA nucleotide sequence of SEQ ID No: 47, as follows:

[SEQ ID No: 47]
ATGACAACTAGAACAAAGGGCAGGGGCCATACTGCGGCCACGACTCAAAA
CGACAGAATGCCAGGCCCTGAGCTTTCGGGCTGGATCTCTGAGCAGCTAA
TGACCGGAAGAATTCCTGTAAGCGACATCTTCTGTGATATTGAGAACAAT
CCAGGATTATGCTACGCATCCCAAATGCAACAAACGAAGCCAAACCCGAA
GACGCGCAACAGTCAAACCCAAACGGACCCAATTTGCAATCATAGTTTTG
AGGAGGTAGTACAAACATTGGCTTCATTGGCTACTGTTGTGCAACAACAA
ACCATCGCATCAGAATCATTAGAACAACGCATTACGAGTCTTGAGAATGG
TCTAAAGCCAGTTTATGATATGGCAAAAACAATCTCCTCATTGAACAGGG
TTTGTGCTGAGATGGTTGCAAAATATGATCTTCTGGTGATGACAACCGGT
CGGGCAACAGCAACCGCTGCGGCAACTGAGGCTTATTGGGCCGAACATGG
TCAACCACCACCTGGACCATCACTTTATGAAGAAAGTGCGATTCGGGGTA
AGATTGAATCGAGGAAAATTTTGGGAAACCTGACATTTCGGCAAAGGATT
TGAGAAACATTATGTATGATCACTTGCCTGGTTTTGGAACTGCTTTCCAC
CAATTAGTACAAGTGATTTGTAAATTGGGAAAAGATAGCAACTCATTGGA
CATCATTCATGCTGAGTTCCAGGCCAGCCTGGCTGAAGGAGACTCTCCTC
AATGTGCCCTAATTCAAATTACAAAAAGAGTTCCAATCTTCCAAGATGCT
GCTCCACCTGTCATCCACATCCGCTCTCGAGGTGACATTCCCCGAGCTTG
CCAGAAAAGCTTGCGTCCAGTCCCACCATCGCCCAAGATTGATCGAGGTT
GGGTATGTGTTTTTCAGCTTCAAGATGGTAAAACACTTGGACTCAAAATT

Accordingly, preferably the Ebola VP35 polypeptide is encoded by the DNA nucleotide sequence substantially as set out in SEQ ID NO: 47, or a variant or fragment thereof.

Thus, the RNA construct may comprise an RNA nucleotide sequence of SEQ ID No: 48, as follows:

[SEQ ID No: 48]
AUGACAACUAGAACAAAGGGCAGGGGCCAUACUGCGGCCACGACUCAAAA
CGACAGAAUGCCAGGCCCUGAGCUUUCGGGCUGGAUCUCUGAGCAGCUAA
UGACCGGAAGAAUUCCUGUAAGCGACAUCUUCUGUGAUAUUGAGAACAAU
CCAGGAUUAUGCUACGCAUCCCAAAUGCAACAAACGAAGCCAAACCCGAA
GACGCGCAACAGUCAAACCCAAACGGACCCAAUUUGCAAUCAUAGUUUUG
AGGAGGUAGUACAAACAUUGGCUUCAUUGGCUACUGUUGUGCAACAACAA
ACCAUCGCAUCAGAAUCAUUAGAACAACGCAUUACGAGUCUUGAGAAUGG
UCUAAAGCCAGUUUAUGAUAUGGCAAAAACAAUCUCCUCAUUGAACAGGG
UUUGUGCUGAGAUGGUUGCAAAAUAUGAUCUUCUGGUGAUGACAACCGGU
CGGGCAACAGCAACCGCUGCGGCAACUGAGGCUUAUUGGGCCGAACAUGG
UCAACCACCACCUGGACCAUCACUUUAUGAAGAAAGUGCGAUUCGGGGUA
AGAUUGAAUCUAGAGAUGAGACCGUCCCUCAAAGUGUUAGGGAGGCAUUC
AACAAUCUAAACAGUACCACUUCACUAACUGAGGAAAAUUUUGGGAAACC
UGACAUUUCGGCAAAGGAUUUGAGAAACAUUAUGUAUGAUCACUUGCCUG
GUUUUGGAACUGCUUUCCACCAAUUAGUACAAGUGAUUUGUAAAUUGGGA
AAAGAUAGCAACUCAUUGGACAUCAUUCAUGCUGAGUUCCAGGCCAGCCU
GGCUGAAGGAGACUCUCCUCAAUGUGCCCUAAUUCAAAUUACAAAAAGAG
UUCCAAUCUUCCAAGAUGCUGCUCCACCUGUCAUCCACAUCCGCUCUCGA
GGUGACAUUCCCCGAGCUUGCCAGAAAAGCUUGCGUCCAGUCCCACCAUC
GCCCAAGAUUGAUCGAGGUUGGGUAUGUGUUUUUCAGCUUCAAGAUGGUA
AAACACUUGGACUCAAAAUU

Furthermore, preferably the RNA construct comprises an RNA nucleotide sequence substantially as set out in SEQ ID No: 48, or a variant or fragment thereof.

The inventors then subjected the protein sequence of SEQ ID No: 46 to codon optimisation for human expression, and one embodiment of the codon optimised nucleic acid (DNA) sequence that includes a start (ATG) and a stop (TGA) codon is provided herein as SEQ ID No: 49, as follows:

[SEQ ID No: 49]
ATGACCACCAGGACCAAAGGCAGAGGACACACCGCCGCCACCACACAGAA
CGACAGAATGCCTGGACCTGAGCTGAGCGGCTGGATCTCTGAGCAGCTGA
TGACAGGCAGAATCCCCGTGTCCGACATCTTCTGCGACATCGAGAACAAC
CCCGGCCTGTGTTACGCCAGCCAGATGCAGCAGACCAAGCCTAATCCTAA
GACACGGAACAGCCAGACACAGACAGACCCCATCTGCAACCACAGCTTCG
AGGAAGTGGTGCAGACACTGGCCAGCCTGGCCTGAAGCCTGTGTACGACA
TGGCCAAGACCATCAGCTCCCTGAACAGAGTGTGCGCCGAGATGGTGGCC
AAATACGACCTGCTCGTGATGACCACCGGCAGAGCCACAGCTACAGCCGC
TGCCACAGAAGCCTATTGGGCCGAACATGGACAGCCTCCACCTGGACCTA
GCCTGTACGAGGAATCTGCCATCCGGGGCAAGATCGAGAGCAGGGATGAG
ACAGTGCCCCAGTCTGTGCGCGAGGCCTTCAACAACCTGAACAGCACCAC
AAGCCTGACCGAGGAAAACTTCGGCAAGCCCGACATCAGCGCCAAGGACC
TGCGGAACATTATGTACGACCATCTGCCTGGCTTCGGCACCGCCTTCCAT
CAGCTGGTGCAAGTGATCTGCAAGCTGGGCAAAGACAGCAACAGCCTGGA
CATCATCCACGCCGAGTTTCAGGCCTCTCTGGCCGAAGGCGATTCTCCTC
AGTGTGCCCTGATCCAGATCACCAAGCGGGTGCCCATCTTCCAGGATGCT
GCCCCTCCTGTGATCCACATCAGAAGCAGAGGCGACATCCCCAGAGCCTG
CCAGAAATCTCTCAGACCCGTGCCTCCATCTCCTAAGATCGACAGAGGCT
GGGTCTGCGTGTTCCAGCTGCAAGATGGCAAGACCCTGGGCCTGAAGATC
TGA

Hence, preferably the RNA construct comprises a sequence substantially as set out in SEQ ID No: 49, or a fragment or variant thereof.

In an embodiment, the RNA sequence corresponding to the codon optimised DNA sequence of SEQ ID No: 49 that includes a start (AUG) and a stop (UGA) codon is provided herein as SEQ ID No: 50, as follows:

[SEQ ID No: 50]
AUGACCACCAGGACCAAAGGCAGAGGACACACCGCCGCCACCACACAGAA
CGACAGAAUGCCUGGACCUGAGCUGAGCGGCUGGAUCUCUGAGCAGCUGA
UGACAGGCAGAAUCCCCGUGUCCGACAUCUUCUGCGACAUCGAGAACAAC
CCCGGCCUGUGUUACGCCAGCCAGAUGCAGCAGACCAAGCCUAAUCCUAA
GACACGGAACAGCCAGACACAGACAGACCCCAUCUGCAACCACAGCUUCG
AGGAAGUGGUGCAGACACUGGCCAGCCUGGCUACAGUUGUGCAGCAGCAG
ACAAUCGCCAGCGAGAGCCUGGAACAGAGAAUCACCAGCCUGGAAAACGG
CCUGAAGCCUGUGUACGACAUGGCCAAGACCAUCAGCUCCCUGAACAGAG
UGUGCGCCGAGAUGGUGGCCAAAUACGACCUGCUCGUGAUGACCACCGGC
AGAGCCACAGCUACAGCCGCUGCCACAGAAGCCUAUUGGGCCGAACAUGG
ACAGCCUCCACCUGGACCUAGCCUGUACGAGGAAUCUGCCAUCCGGGGCA
AGAUCGAGAGCAGGGAUGAGACAGUGCCCCAGUCUGUGCGCGAGGCCUUC
AACAACCUGAACAGCACCACAAGCCUGACCGAGGAAAACUUCGGCAAGCC
CGACAUCAGCGCCAAGGACCUGCGGAACAUUAUGUACGACCAUCUGCCUG
GCUUCGGCACCGCCUUCCAUCAGCUGGUGCAAGUGAUCUGCAAGCUGGGC
AAAGACAGCAACAGCCUGGACAUCAUCCACGCCGAGUUUCAGGCCUCUCU
GGCCGAAGGCGAUUCUCCUCAGUGUGCCCUGAUCCAGAUCACCAAGCGGG
UGCCCAUCUUCCAGGAUGCUGCCCCUCCUGUGAUCCACAUCAGAAGCAGA
GGCGACAUCCCCAGAGCCUGCCAGAAAUCUCUCAGACCCGUGCCUCCAUC
UCCUAAGAUCGACAGAGGCUGGGUCUGCGUGUUCCAGCUGCAAGAUGGCA
AGACCCUGGGCCUGAAGAUCUGA

Hence, preferably the RNA construct comprises a sequence substantially as set out in SEQ ID No: 50, or a fragment or variant thereof.

In another embodiment, the at least one IIP may be derived from SARS-CoV-2 ORF3b (Accession Number—NCBI Reference Sequence: NC_045512.2) or an orthologue thereof. One embodiment of the wild type of SARS-CoV-2 ORF3b polypeptide sequence from which the at least one IIP may be derived is represented herein as SEQ ID No:51. The asterisks represent stop codons in the wild-type sequence, one or more of which may be mutated in the derived IIP. SEQ ID No:51 is as follows:

[SEQ ID No: 51]
MMPTIFFAGILIVTTIVYLTIV*LLQLSLLQVMAQQVLFLNMTTRLVVIL
KNGNLE*KTVLYYTVTSLQTITSCTQLN*VQTLVLNMLPSSSTIKLLMSL
KNMSKFTQSTVHPELLIQ*WNQFMMNRRRLLACLCKHKLMSTNLCTHSFR
KRQVR*

In one embodiment, the at least one IIP may be an ORF3b*57 variant of the wild type of SARS-CoV-2 ORF3b, or an orthologue thereof. One embodiment of the SARS-CoV-2 ORF3b*57 variant polypeptide sequence is represented herein as SEQ ID No:52, as follows:

[SEQ ID No: 52]
MMPTIFFAGILIVTTIVYLTIVQLLQLSLLQVMAQQVLFLNMTTRLVVIL
KNGNLE

Therefore, preferably the RNA construct of the first aspect comprises a nucleotide sequence which encodes an amino acid sequence substantially as set out in SEQ ID No: 52, or a variant or fragment thereof.

In one embodiment, the SARS-CoV-2 ORF3b*57 variant polypeptide is encoded by the DNA nucleotide sequence of SEQ ID No: 53, as follows:

[SEQ ID No: 53]
ATGATGCCAACTATTTTCTTTGCTGGCATACTAATTGTTACGACTATTGT
ATACCTTACAATAGTGCAACTTCTTCAATTGTCATTACTTCAGGTGATGG
CACAACAAGTCCTATTTCTGAACATGACTACCAGATTGGTGGTTATACTG
AAAAATGGGAATCTGGAG

Accordingly, preferably the SARS-CoV-2 ORF3b*57 variant polypeptide is encoded by the DNA nucleotide sequence substantially as set out in SEQ ID NO: 53, or a variant or fragment thereof.

Thus, the RNA construct may comprise an RNA nucleotide sequence of SEQ ID No: 54, as follows:

[SEQ ID No: 54]
AUGAUGCCAACUAUUUUCUUUGCUGGCAUACUAAUUGUUACGACUAUUGU
AUACCUUACAAUAGUGCAACUUCUUCAAUUGUCAUUACUUCAGGUGAUGG
CACAACAAGUCCUAUUUCUGAACAUGACUACCAGAUUGGUGGUUAUACUG
AAAAAUGGGAAUCUGGAG

Furthermore, preferably the RNA construct comprises an RNA nucleotide sequence substantially as set out in SEQ ID No: 54, or a variant or fragment thereof.

The inventors then subjected the protein sequence of SEQ ID No: 52 to codon optimisation for human expression, and one embodiment of the codon optimised nucleic acid (DNA) sequence that includes a start (ATG) and a stop (TGA) codon is provided herein as SEQ ID No: 55, as follows:

[SEQ ID No: 55]
ATGATGCCGACCATCTTCTTCGCCGGCATCCTGATCGTGACCACCATCGT
GTACCTGACCATCGTGCAGCTGCTGCAGCTCAGCCTGCTGCAAGTGATGG
CTCAGCAGGTCCTGTTCCTGAATATGACCACCAGACTGGTCGTGATCCTG
AAGAACGGCAACCTGGAATGA

Hence, preferably the RNA construct is encoded by a DNA sequence substantially as set out in SEQ ID No: 55, or a fragment or variant thereof.

In an embodiment, the RNA sequence corresponding to the codon optimised DNA is sequence of SEQ ID No: 55 that includes a start (AUG) and a stop (UGA) codon is provided herein as SEQ ID No: 56, as follows:

[SEQ ID No: 56]
AUGAUGCCGACCAUCUUCUUCGCCGGCAUCCUGAUCGUGACCACCAUCGU
GUACCUGACCAUCGUGCAGCUGCUGCAGCUCAGCCUGCUGCAAGUGAUGG
CUCAGCAGGUCCUGUUCCUGAAUAUGACCACCAGACUGGUCGUGAUCCUG
AAGAACGGCAACCUGGAAUGA

Hence, preferably the RNA construct comprises a sequence substantially as set out in SEQ ID No: 56, or a fragment or variant thereof.

In another embodiment, the at least one IIP may be an ORF3b*79 variant of the wild type of SARS-CoV-2 ORF3b, or an orthologue thereof. One embodiment of the SARS-CoV-2 ORF3b*79 variant polypeptide sequence is represented herein as SEQ ID No:57, as follows:

[SEQ ID No: 57]
MMPTIFFAGILIVTTIVYLTIVQLLQLSLLQVMAQQVLFLNMTTRLVVIL
KNGNLELKTVLYYTVTSLQTITSCTQLN

Therefore, preferably the RNA construct of the first aspect comprises a nucleotide sequence which encodes an amino acid sequence substantially as set out in SEQ ID No: 57, or a variant or fragment thereof.

In one embodiment, the SARS-CoV-2 ORF3b*79 variant polypeptide is encoded by the DNA nucleotide sequence of SEQ ID No: 58, as follows:

[SEQ ID No: 58]
ATGATGCCAACTATTTTCTTTGCTGGCATACTAATTGTTACGACTATTGT
ATACCTTACAATAGTGCAACTTCTTCAATTGTCATTACTTCAGGTGATGG
CACAACAAGTCCTATTTCTGAACATGACTACCAGATTGGTGGTTATACTG
AAAAATGGGAATCTGGAGTTAAAGACTGTGTTGTATTACACAGTTACTTC
ACTTCAGACTATTACCAGCTGTACTCAACTCAAT

Accordingly, preferably the SARS-CoV-2 ORF3b*79 variant polypeptide is encoded by the DNA nucleotide sequence substantially as set out in SEQ ID NO: 58, or a variant or fragment thereof.

Thus, the RNA construct may comprise an RNA nucleotide sequence of SEQ ID No: 59, as follows:

[SEQ ID No: 59]
AUGAUGCCAACUAUUUUCUUUGCUGGCAUACUAAUUGUUACGACUAUUGU
AUACCUUACAAUAGUGCAACUUCUUCAAUUGUCAUUACUUCAGGUGAUGG
CACAACAAGUCCUAUUUCUGAACAUGACUACCAGAUUGGUGGUUAUACUG
AAAAAUGGGAAUCUGGAGUUAAAGACUGUGUUGUAUUACACAGUUACUUC
ACUUCAGACUAUUACCAGCUGUACUCAACUCAAU

Furthermore, preferably the RNA construct comprises an RNA nucleotide sequence substantially as set out in SEQ ID No: 59, or a variant or fragment thereof.

The inventors then subjected the protein sequence of SEQ ID No: 57 to codon optimisation for human expression, and one embodiment of the codon optimised nucleic acid (DNA) sequence that includes a start (ATG) and a stop (TGA) codon is provided herein as SEQ ID No: 60, as follows:

[SEQ ID No: 60]
ATGATGCCGACCATCTTCTTCGCCGGCATCCTGATCGTGACCACCATCGT
GTACCTGACCATCGTGCAGCTGCTGCAGCTCAGCCTGCTGCAAGTGATGG
CTCAGCAGGTCCTGTTCCTGAATATGACCACCAGACTGGTCGTGATCCTG
AAGAACGGCAACCTGGAACTGAAAACCGTGCTGTACTACACCGTGACCAG
CCTGCAGACCATCACCAGCTGCACCCAGCTGAACTGA

Hence, preferably the RNA construct is encoded by a DNA sequence substantially as set out in SEQ ID No: 60, or a fragment or variant thereof.

In an embodiment, the RNA sequence corresponding to the codon optimised DNA sequence of SEQ ID No: 60 that includes a start (AUG) and a stop (UGA) codon is provided herein as SEQ ID No: 61, as follows:

[SEQ ID No: 61]
AUGAUGCCGACCAUCUUCUUCGCCGGCAUCCUGAUCGUGACCACCAUCGU
GUACCUGACCAUCGUGCAGCUGCUGCAGCUCAGCCUGCUGCAAGUGAUGG
CUCAGCAGGUCCUGUUCCUGAAUAUGACCACCAGACUGGUCGUGAUCCUG
AAGAACGGCAACCUGGAACUGAAAACCGUGCUGUACUACACCGUGACCAG
CCUGCAGACCAUCACCAGCUGCACCCAGCUGAACUGA

Hence, preferably the RNA construct comprises a sequence substantially as set out in SEQ ID No: 61, or a fragment or variant thereof.

In yet another embodiment, the at least one IIP may be an ORF3b*57 Ecuador variant of the wild type of SARS-CoV-2 ORF3b, or an orthologue thereof. One embodiment of the SARS-CoV-2 ORF3b*57 Ecuador variant polypeptide sequence is represented herein as SEQ ID No:62, as follows:

[SEQ ID No: 62]
MMPTIFFAGILIVTTIVYLTIVQMLQLSLLQVMAQQVLFLNMTTRLVVIL
KNGNLE

Therefore, preferably the RNA construct of the first aspect comprises a nucleotide sequence which encodes an amino acid sequence substantially as set out in SEQ ID No: 62, or a variant or fragment thereof.

In one embodiment, the SARS-CoV-2 ORF3b*57 Ecuador variant polypeptide is encoded by the DNA nucleotide sequence of SEQ ID No: 63, as follows:

[SEQ ID No: 63]
ATGATGCCAACTATTTTCTTTGCTGGCATACTAATTGTTACGACTATTGT
ATACCTTACAATAGTGCAAATGCTTCAATTGTCATTACTTCAGGTGATGG
CACAACAAGTCCTATTTCTGAACATGACTACCAGATTGGTGGTTATACTG
AAAAATGGGAATCTGGAGTAA

Accordingly, preferably the SARS-CoV-2 ORF3b*57 Ecuador variant polypeptide is encoded by the DNA nucleotide sequence substantially as set out in SEQ ID NO: 63, or a variant or fragment thereof.

Thus, the RNA construct may comprise an RNA nucleotide sequence of SEQ ID No: 64, as follows:

[SEQ ID No: 64]
AUGAUGCCAACUAUUUUCUUUGCUGGCAUACUAAUUGUUACGACUAUUGU
AUACCUUACAAUAGUGCAAAUGCUUCAAUUGUCAUUACUUCAGGUGAUGG
CACAACAAGUCCUAUUUCUGAACAUGACUACCAGAUUGGUGGUUAUACUG
AAAAAUGGGAAUCUGGAGUAA

Furthermore, preferably the RNA construct comprises an RNA nucleotide sequence substantially as set out in SEQ ID No: 64, or a variant or fragment thereof.

The inventors then subjected the protein sequence of SEQ ID No: 62 to codon optimisation for human expression, and one embodiment of the codon optimised nucleic acid (DNA) sequence that includes a start (ATG) and a stop (TGA) codon is provided herein as SEQ ID No: 65, as follows:

[SEQ ID No: 65]
ATGATGCCGACCATCTTCTTCGCCGGCATCCTGATCGTGACCACCATCGT
GTACCTGACCATCGTGCAGATGCTGCAGCTGAGCCTGCTGCAAGTGATGG
CCCAGCAGGTCCTGTTCCTGAATATGACCACCAGACTGGTCGTGATCCTG
AAGAACGGCAACCTGGAATGA

Hence, preferably the RNA construct is encoded by a DNA sequence substantially as set out in SEQ ID No: 65, or a fragment or variant thereof.

In an embodiment, the RNA sequence corresponding to the codon optimised DNA sequence of SEQ ID No: 65 that includes a start (AUG) and a stop (UGA) codon is provided herein as SEQ ID No: 66, as follows:

[SEQ ID No: 66]
AUGAUGCCGACCAUCUUCUUCGCCGGCAUCCUGAUCGUGACCACCAUCGU
GUACCUGACCAUCGUGCAGAUGCUGCAGCUGAGCCUGCUGCAAGUGAUGG
CCCAGCAGGUCCUGUUCCUGAAUAUGACCACCAGACUGGUCGUGAUCCUG
AAGAACGGCAACCUGGAAUGA

Hence, preferably the RNA construct comprises a sequence substantially as set out in SEQ ID No: 66, or a fragment or variant thereof.

In other embodiments, the at least one IIP may be a viral inhibitory protein which blocks or inhibits the activity of MDA-5, and selected from a group consisting of:
(i) a Paromyxoviridae V protein; optionally SV5B, PIV2, Mumps, SeV, Measles, or NiV;
(ii) Encephalomyocarditis virus 2C;
(iii) Birnavirus VP3; and
(iv) Porcine delta coronavirus NS6.

In other embodiments, the at least one IIP may be an Arenavirus Z protein selected from a group consisting of: LASV; DANV; LCMV; LUJV; CHPV; MACV; GTOV; JUNV; and SABV. These IIPs are believed to interact with the RLR, RIG-I and MDA-5 and inhibit interaction with mitochondrial antiviral signaling (MAVS).

In other embodiments, the at least one IIP may be a 3C protease protein selected from a group consisting of: Cosackieviruses CV-A16 or CV-A6; EV D-68 or 71; and Poliovirus HEV-C. These IIPs are believed to inhibit MDA interaction with MAVS.

In other embodiments, the at least one IIP may be a 2A protease protein selected from a group consisting of: Poliovirus HEV-C; CVB3; and EV71. These IIPs are believed to inhibit the RLR pathway by cleavage of MDA-5 and MAVS.

In other embodiments, the at least one IIP may be a protein degrading or acting on RIG-I and selected from a group consisting of: hMPV G; CVB3 3C protease; Polio Virus 3Cpro; Hepatitis C NS3/4A; DENV NS3; EV71 3Cpro; FMDV Lpro and 3Cpro; Toscana Virus NSS; Influenza A/PR/8/34 NS1; and MERS ORF8b. These IIPs are preferred in the embodiment in which the RNA construct comprises saRNA or mRNA.

In other embodiments, the at least one IIP may be a protein acting on PACT selected from a group consisting of: SARS CoV N protein; and MHV N.

In other embodiments, the at least one IIP may be a protein acting against LGP2 (which enhances MDA2 signalling), including, for example, FMDV Lpro, 3Cpro and 2B.

In other embodiments, the at least one IIP may be a protein that impacts activity of TRAF3, including, for example, MERS CoV M.

In other embodiments, the at least one IIP may be a protein which acts on MAVS selected from a group consisting of: Influenza virus A PB1-F2; Hepatitis A ABC; Hepatitis B X; Hepatitis C NS3/4A; SARS-CoV NSP15; SARS CoV ORF9 (96); Coxsackievirus B3 2Apro and 3Cpro; Rhinovirus 2Apro and 3Cpro; Rotavirus VP3; GB Virus B NS3/4A; EV71 2Apro; HAV 3Cpro; Human metapneumovirus M2-2; and Enterovirus EV71 2A.

In other embodiments, the at least one IIP may be a protein that blocks IRF-3 activity selected from a group consisting of: HCV and NSP1B; Ebola vNS3; HPV16 E6; Hepatitis E methyltransferase; PRV Npro; HSV1 Us3; HSV2 Us1; CSFV Npro; BDV Npro; Bovine RV NS1 and NS2; Hepatitis B virus Orf3; PBoV NP1; Hepatitis E ORF3; and MERS ORF8b.

In other embodiments, the at least one IIP may be a protein that inhibits IRF7 activity selected from a group consisting of: Rotavirus NSP1; KSHV ORF45; EBV BZLF-1; Ebola VP35; and Enterovirus 71 and 68 3CPro.

In other embodiments, the at least one IIP may be a protein that impacts NF-kβ activity selected from a group consisting of: Polio 3C; FMDV Lpro; MERS CoV ORF4b (246aa); MuHV ORF73; Torque Teno virus ORF2; EBV EBNA1; and SV5 and hPIV2 Vproteins.

In other embodiments, the at least one IIP may be a protein that impacts TBK-1 or IKKε activities and selected from a group consisting of: BDV P; HPV E6 and HPV E7; Arenavirus NP1; HCV NS3 protein; DENV1 NS4A; DENV1, 2 and 4 NS2A and NS2B; WNV NS4A; Ebola VP35; Rabies Virus PP; PEDV N protein; and HSV1 ICP27 and VP24.

In other embodiments, the at least one IIP may be a protein that impacts on the activity of NEMO, such as for example, murine CTMV M45.

The following viral IIPs are believed to block or inhibit activation of MDA-5.

The V protein binds to the helicase domain of MDA-5 and blocks its activation by inhibiting dsRNA binding and consequent self-association. (Childs K S, Andresjeva J, Randall R E & Goobourn S (2009) Mechanism of MDA-5 inhibition by paramyxovirus V proteins. J Virol 83, 3, 1465-1473) In one embodiment, the at least one IIP is SV5B, or an orthologue thereof. One embodiment of the polypeptide sequence of Simian Virus (PIV5 Non-structural protein V) is represented herein as SEQ ID No: 95, as follows:

[SEQ ID No: 95]
MDPTDLSFSPDEINKLIETGLNTVEYFTSQQVTGTSSLGKNTIPPGVTGL
LTNAAEAKIQESTNHQKGSVGGGAKPKKPRPKIAIVPADDKTVPGKPIPN
PLLGLDSTPSTQTVLDLSGKTLPSGSYKGVKLAKFGKENLMTRFIEEPRE
NPIATSSPIDFKRGRDIGGFHRREYSIGWVGDEVKVTEWCNPSCSPITAA
ARRFECTCHQCPVTCSECERDT

Therefore, preferably the RNA construct comprises a nucleotide sequence which encodes an amino acid sequence substantially as set out in SEQ ID No: 95, or a variant or fragment thereof.

In one embodiment, the SV5B polypeptide is encoded by the DNA nucleotide sequence of SEQ ID No: 96, as follows:

[SEQ ID No: 96]
ATGGATCCCACTGATCTGAGCTTCTCCCCAGATGAGATCAATAAGCTCAT
AGAGACAGGCCTGAATACTGTAGAGTATTTTACTTCCCAACAAGTCACAG
GAACATCCTCTCTTGGAAAGAATACAATACCACCAGGGGTCACAGGACTA
CTAACCAATGCTGCAGAGGCAAAGATCCAAGAGTCAACTAACCATCAGAA
GGGCTCAGTTGGTGGGGGTGCAAAACCAAAGAAACCGCGACCAAAAATTG
CCATTGTGCCAGCAGATGACAAAACAGTGCCCGGAAAGCCGATCCCAAAC
CCTCTATTAGGTCTGGACTCCACCCCGAGCACCCAAACTGTGCTTGATCT
AAGTGGGAAAACATTACCATCAGGATCCTATAAGGGGGTTAAGCTTGCGA
AATTTGGAAAAGAAAATCTGATGACACGGTTCATCGAGGAACCCAGAGAG
AATCCTATCGCAACCAGTTCCCCCATCGATTTTAAGAGGGGCAGGGATAC
CGGCGGGTTCCATAGAAGGGAGTACTCAATCGGATGGGTGGGAGATGAAG
TCAAGGTCACTGAGTGGTGCAATCCATCCTGTTCTCCAATCACCGCTGCA
GCAAGGCGATTTGAATGCACTTGTCACCAGTGTCCAGTCACTTGCTCTGA
ATGTGAACGAGATACT

Accordingly, preferably the SV5B polypeptide is encoded by the DNA nucleotide sequence substantially as set out in SEQ ID NO: 96, or a variant or fragment thereof.

One embodiment of codon optimised nucleic acid (DNA) encoding the SV5B polypeptide is provided herein as SEQ ID No: 97, as follows:

[SEQ ID No: 97]
ATGGACCCTACCGACCTGAGCTTCAGCCCCGACGAGATCAACAAGCTGAT
CGAGACAGGCCTGAACACCGTGGAATACTTCACCAGCCAGCAAGTGACCG
GCACAAGCAGCCTGGGCAAGAACACAATTCCTCCAGGCGTGACCGGCCTG
CTGACAAATGCTGCCGAGGCCAAGATCCAAGAGAGCACCAACCACCAGAA
GGGCTCTGTTGGAGGCGGAGCCAAGCCTAAGAAGCCCAGACCTAAGATCG
CCATCGTGCCCGCCGACGATAAGACAGTGCCTGGCAAGCCCATTCCTAAT
CCTCTGCTGGGCCTCGACAGCACCCCTAGCACACAGACAGTGCTGGATCT
GAGCGGCAAGACACTGCCTAGCGGCAGCTATAAGGGCGTGAAGCTGGCCA
AGTTCGGCAAAGAAAACCTGATGACCCGGTTCATCGAGGAACCCAGAGAG
AACCCTATCGCCACCAGCTCTCCCATCGACTTCAAGAGAGGCAGAGACAC
CGGCGGCTTCCACAGAAGAGAGTACAGCATTGGCTGGGTCGGAGATGAAG
TGAAAGTGACCGAGTGGTGCAACCCCAGCTGCAGCCCTATTACAGCCGCC
GCTAGAAGATTCGAGTGCACCTGTCACCAGTGTCCTGTGACCTGTAGCGA
GTGCGAGAGAGACACA

Hence, preferably the RNA construct is encoded by a DNA sequence substantially as set out in SEQ ID No: 97, or a fragment or variant thereof.

In an embodiment, the RNA sequence corresponding to the codon optimised DNA

[SEQ ID No: 98]
AUGGACCCUACCGACCUGAGCUUCAGCCCCGACGAGAUCAACAAGCUGAU
CGAGACAGGCCUGAACACCGUGGAAUACUUCACCAGCCAGCAAGUGACCG
GCACAAGCAGCCUGGGCAAGAACACAAUUCCUCCAGGCGUGACCGGCCUG
CUGACAAAUGCUGCCGAGGCCAAGAUCCAAGAGAGCACCAACCACCAGAA
GGGCUCUGUUGGAGGCGGAGCCAAGCCUAAGAAGCCCAGACCUAAGAUCG
CCAUCGUGCCCGCCGACGAUAAGACAGUGCCUGGCAAGCCCAUUCCUAAU
CCUCUGCUGGGCCUCGACAGCACCCCUAGCACACAGACAGUGCUGGAUCU
GAGCGGCAAGACACUGCCUAGCGGCAGCUAUAAGGGCGUGAAGCUGGCCA
AGUUCGGCAAAGAAAACCUGAUGACCCGGUUCAUCGAGGAACCCAGAGAG
AACCCUAUCGCCACCAGCUCUCCCAUCGACUUCAAGAGAGGCAGAGACAC
CGGCGGCUUCCACAGAAGAGAGUACAGCAUUGGCUGGGUCGGAGAUGAAG
UGAAAGUGACCGAGUGGUGCAACCCCAGCUGCAGCCCUAUUACAGCCGCC
GCUAGAAGAUUCGAGUGCACCUGUCACCAGUGUCCUGUGACCUGUAGCGA
GUGCGAGAGAGACACA

Hence, preferably the RNA construct comprises a sequence substantially as set out in SEQ ID No: 98, or a fragment or variant thereof.

In one embodiment, the at least one IIP is PIV2 Non-structural protein V (P19847), or an orthologue thereof. One embodiment of the polypeptide sequence of PIV2 is represented herein as SEQ ID No: 99, as follows:

[SEQ ID No: 99]
MAEEPTYTTEQVDELIHAGLGTVDFFLSRPIDAQSSLGKGSIPPGVTAVL
TSAAEAKSKPVAAGPVKPRRKKVISNTTPYTIADNIPPEKLPINTPIPNP
LLPLARPHGKMTDIDIVTGNITEGSYKGVELAKLGKQTLLTRFTSNEPVS
SAGSAQDPNFKRGGANRERARGNHRREWSIAWVGDQVKVFEWCNPRCAPV
TASARKFTCTCGSCPSICGECEGDH

Therefore, preferably the RNA construct comprises a nucleotide sequence which encodes an amino acid sequence substantially as set out in SEQ ID No: 99, or a variant or fragment thereof.

In one embodiment, the PIV2 polypeptide is encoded by the DNA nucleotide sequence of SEQ ID No: 100, as follows:

[SEQ ID No: 100]
ATGGCCGAGGAACCAACATACACCACTGAGCAAGTTGATGAATTAATCCA
TGCTGGACTGGGAACAGTAGATTTCTTCCTATCTAGACCCATAGATGCTC
AGTCTTCTTTAGGCAAAGGCAGCATCCCACCAGGTGTCACAGCTGTTCTA
ACTAGTGCAGCGGAGGCAAAATCCAAACCAGTTGCTGCTGGTCCAGTTAA
ACCCAGGCGGAAGAAAGTGATCAGCAATACTACTCCATACACTATTGCAG
ACAATATTCCACCTGAGAAGCTACCGATCAACACTCCAATACCCAATCCA
TTACTTCCACTGGCACGCCCTCACGGAAAGATGACAGACATTGACATTGT
CACTGGGAACATTACAGAAGGATCGTACAAAGGTGTGGAGCTTGCTAAAT
TAGGGAAGCAGACACTACTCACAAGGTTCACCTCGAATGAGCCAGTCTCC
TCAGCTGGATCCGCCCAAGACCCCAACTTTAAGAGGGGGGGAGCTAATAG
AGAAAGAGCAAGAGGCAACCATAGGAGAGAATGGAGTATTGCATGGGTCG
GAGATCAGGTCAAAGTCTTCGAGTGGTGTAATCCCAGGTGTGCCCCAGTC
ACGGCCTCAGCTCGCAAGTTCACCTGCACATGCGGATCCTGCCCCAGCAT
CTGCGGAGAATGTGAAGGAGATCAT

Accordingly, preferably the PIV2 polypeptide is encoded by the DNA nucleotide sequence substantially as set out in SEQ ID NO: 100, or a variant or fragment thereof.

One embodiment of codon optimised nucleic acid (DNA) encoding the PIV2 polypeptide is provided herein as SEQ ID No: 101, as follows:

[SEQ ID No: 101]
ATGGCCGAGGAACCTACCTACACCACCGAACAGGTGGACGAGCTGATTCA
CGCCGGACTGGGAACCGTGGACTTCTTTCTGTCCCGGCCTATCGATGCCC
AGAGCAGCCTCGGCAAGGGATCTATTCCTCCTGGCGTGACAGCCGTGCTG
ACATCTGCCGCCGAGGCCAAGTCTAAACCTGTGGCTGCTGGACCCGTGAA
GCCCAGACGGAAGAAAGTGATCAGCAACACCACACCTTACACGATCGCCG
ACAACATCCCTCCTGAGAAGCTGCCCATCAACACCCCTATTCCTAATCCT
CTGCTGCCCCTGGCCAGACCTCACGGCAAGATGACCGACATCGATATCGT
GACCGGCAACATCACCGAGGGCAGCTACAAAGGCGTGGAACTGGCCAAGC
TGGGCAAGCAGACACTGCTGACCAGATTCACCAGCAACGAGCCTGTGTCT
AGCGCCGGCTCTGCCCAGGATCCTAACTTCAAAAGAGGCGGAGCCAACAG
AGAGAGAGCCAGAGGCAACCATCGGAGAGAGTGGTCTATTGCCTGGGTCG
GAGATCAAGTGAAGGTGTTCGAGTGGTGCAACCCCAGATGTGCCCCTGTG
ACAGCCAGCGCCAGAAAGTTCACCTGTACCTGCGGCAGCTGTCCCAGCAT
TTGCGGAGAGTGTGAAGGCGACCAT

Hence, preferably the RNA construct is encoded by a DNA sequence substantially as set out in SEQ ID No: 101, or a fragment or variant thereof.

In an embodiment, the RNA sequence corresponding to the codon optimised DNA sequence of SEQ ID No: 101 is provided herein as SEQ ID No: 102, as follows:

[SEQ ID No: 102]
AUGGCCGAGGAACCUACCUACACCACCGAACAGGUGGACGAGCUGAUUCA
CGCCGGACUGGGAACCGUGGACUUCUUUCUGUCCCGGCCUAUCGAUGCCC
AGAGCAGCCUCGGCAAGGGAUCUAUUCCUCCUGGCGUGACAGCCGUGCUG
ACAUCUGCCGCCGAGGCCAAGUCUAAACCUGUGGCUGCUGGACCCGUGAA
GCCCAGACGGAAGAAAGUGAUCAGCAACACCACACCUUACACGAUCGCCG
ACAACAUCCCUCCUGAGAAGCUGCCCAUCAACACCCCUAUUCCUAAUCCU
CUGCUGCCCCUGGCCAGACCUCACGGCAAGAUGACCGACAUCGAUAUCGU
GACCGGCAACAUCACCGAGGGCAGCUACAAAGGCGUGGAACUGGCCAAGC
UGGGCAAGCAGACACUGCUGACCAGAUUCACCAGCAACGAGCCUGUGUCU
AGCGCCGGCUCUGCCCAGGAUCCUAACUUCAAAAGAGGCGGAGCCAACAG
AGAGAGAGCCAGAGGCAACCAUCGGAGAGAGUGGUCUAUUGCCUGGGUCG
GAGAUCAAGUGAAGGUGUUCGAGUGGUGCAACCCCAGAUGUGCCCCUGUG
ACAGCCAGCGCCAGAAAGUUCACCUGUACCUGCGGCAGCUGUCCCAGCAU
UUGCGGAGAGUGUGAAGGCGACCAU

Hence, preferably the RNA construct comprises a sequence substantially as set out in SEQ ID No: 102, or a fragment or variant thereof.

In one embodiment, the at least one IIP is Mumps Non-structural protein V (P30928), or an orthologue thereof. One embodiment of the polypeptide sequence of Mumps V protein is represented herein as SEQ ID No: 103, as follows:

[SEQ ID No: 103]
MDQFIKQDETGDLIETGMNVANHFLSAPIQGTNSLSKATIIPGVAPVLIG
NPEQKNIQYPTTSHQGSKSKGRGSGARPIIVSSSEGGTGGTQVPEPLFAQ
TGOGGIVTTVYQDPTIQPTGSYRSVELAKIGKERMINRFVEKPRTSTPVT
EFKRGAGSGCSRPDNPRGGHRREWSLSWVQGEVRVFEWCNPICSPITAAA
RFHSCKCGNCPAKCDQCERDYGPP

Therefore, preferably the RNA construct comprises a nucleotide sequence which encodes an amino acid sequence substantially as set out in SEQ ID No: 103, or a variant or fragment thereof.

In one embodiment, the Mumps V polypeptide is encoded by the DNA nucleotide sequence of SEQ ID No: 104, as follows:

[SEQ ID No: 104]
ATGGACCAATTTATAAAACAAGATGAGACTGGTGATTTAATTGAGACAGG
AATGAACGTTGCAAATCATTTCCTATCCGCCCCCATTCAGGGAACCAACT
CGTTGAGCAAGGCCACAATCATCCCTGGCGTTGCACCAGTACTCATTGGC
AATCCAGAGCAAAAGAACATTCAGTACCCCACCACATCACATCAGGGATC
CAAGTCAAAGGGCAGAGGCTCAGGGGCCAGGCCCATCATAGTCTCATCCT
CCGAAGGAGGCACTGGAGGGACTCAGGTTCCTGAGCCCCTTTTCGCACAA
ACAGGACAAGGTGGCATTGTCACCACCGTTTATCAGGATCCAACTATCCA
ACCAACAGGTTCATATCGAAGTGTGGAATTGGCTAAGATAGGAAAAGAGA
GAATGATTAATCGATTTGTTGAAAAACCAAGAACCTCAACGCCGGTAACA
GAATTTAAGAGGGGGGCCGGGAGCGGCTGCTCAAGGCCAGACAATCCAAG
AGGAGGGCATAGACGGGAATGGAGCCTCAGCTGGGTCCAAGGAGAGGTCC
GGGTCTTTGAGTGGTGCAACCCCATATGCTCACCTATCACTGCCGCAGCA
AGATTCCACTCCTGCAAATGTGGGAATTGCCCCGCAAAGTGCGATCAGTG
CGAACGAGATTATGGACCTCCT

Accordingly, preferably the Mumps V polypeptide is encoded by the DNA nucleotide sequence substantially as set out in SEQ ID NO: 104, or a variant or fragment thereof.

One embodiment of codon optimised nucleic acid (DNA) encoding the Mumps V polypeptide is provided herein as SEQ ID No: 105, as follows:

[SEQ ID No: 105]
ATGGACCAGTTCATCAAGCAGGACGAGACAGGCGACCTGATCGAAACCGG
CATGAACGTGGCCAACCACTTCCTGTCTGCCCCTATCCAGGGCACCAACA
GCCTGAGCAAGGCCACAATTATCCCTGGCGTGGCCCCTGTGCTGATCGGC
AATCCTGAGCAGAAGAACATTCAGTACCCCACCACCAGCCACCAGGGCAG
CAAGTCTAAAGGCAGAGGCTCTGGCGCTCGGCCCATCATCGTTTCTAGTA
GCGAAGGCGGCACCGGCGGAACACAGGTTCCAGAACCTCTGTTTGCCCAG
ACAGGCCAAGGCGGCATCGTGACCACAGTGTACCAGGATCCTACCATCCA
GCCTACCGGCAGCTACAGAAGCGTGGAACTGGCCAAGATCGGCAAAGAAC
GGATGATCAACCGCTTCGTGGAAAAGCCCAGAACCAGCACACCCGTGACC
GAGTTCAAAAGAGGCGCCGGAAGCGGCTGCAGCAGACCCGATAATCCTAG
AGGCGGCCATCGGAGAGAGTGGTCCCTGTCTTGGGTTCAGGGCGAAGTGC
GGGTGTTCGAGTGGTGCAATCCTATCTGCAGCCCCATCACAGCCGCCGCT
AGATTCCACAGCTGCAAGTGCGGAAACTGCCCCGCCAAGTGTGACCAGTG
CGAGAGAGATTACGGCCCTCCT

Hence, preferably the RNA construct is encoded by a DNA sequence substantially as set out in SEQ ID No: 105, or a fragment or variant thereof.

In an embodiment, the RNA sequence corresponding to the codon optimised DNA sequence of SEQ ID No: 105 is provided herein as SEQ ID No: 106, as follows:

[SEQ ID No: 106]
AUGGACCAGUUCAUCAAGCAGGACGAGACAGGCGACCUGAUCGAAACCGGCAUGAACGUGGCCAACCACUUCCUGUCU
GCCCCUAUCCAGGGCACCAACAGCCUGAGCAAGGCCACAAUUAUCCCUGGCGUGGCCCCUGUGCUGAUCGGCAAUCCU
GAGCAGAAGAACAUUCAGUACCCCACCACCAGCCACCAGGGCAGCAAGUCUAAAGGCAGAGGCUCUGGCGCUCGGCCC
AUCAUCGUUUCUAGUAGCGAAGGCGGCACCGGCGGAACACAGGUUCCAGAACCUCUGUUUGCCCAGACAGGCCAAGGC
GGCAUCGUGACCACAGUGUACCAGGAUCCUACCAUCCAGCCUACCGGCAGCUACAGAAGCGUGGAACUGGCCAAGAUC
GGCAAAGAACGGAUGAUCAACCGCUUCGUGGAAAAGCCCAGAACCAGCACACCCGUGACCGAGUUCAAAAGAGGCGCC
GGAAGCGGCUGCAGCAGACCCGAUAAUCCUAGAGGCGGCCAUCGGAGAGAGUGGUCCCUGUCUUGGGUUCAGGGCGAA
GUGCGGGUGUUCGAGUGGUGCAAUCCUAUCUGCAGCCCCAUCACAGCCGCCGCUAGAUUCCACAGCUGCAAGUGCGGA
AACUGCCCCGCCAAGUGUGACCAGUGCGAGAGAGAUUACGGCCCUCCU

Hence, preferably the RNA construct comprises a sequence substantially as set out in SEQ ID No: 106, or a fragment or variant thereof.

In one embodiment, the at least one IIP is Sendai Virus Protein V (strain Fushimi) (P69284), or an orthologue thereof. One embodiment of the polypeptide sequence of Sendai Virus Protein V (SeV V protein) is represented herein as SEQ ID No: 107, as follows:

[SEQ ID No: 107]
MDQDAFILKEDSEVEREAPGGRESLSDVIGFLDAVLSSEPTDIGGDRSWLHNTINTPQGPGSAHRAKSEGEGEVSTPS
TQDNRSGEESRVSGRTSKPEAEAHAGNLDKQNIHRAFGGRTGTNSVSQDLGDGGDSGILENPPNERGYPRSGIEDENR
EMAAHPDKRGEDQAEGLPEEVRGGTSLPDEGEGGASNNGRSMEPGSSHSARVTGVLVIPSPELEEAVLRRNKRRPTNS
GSKPLTPATVPGTRSPPLNRYNSTGSPPGKPPSTQDEHINSGDTPAVRVKDRKPPIGTRSVSDCPANGRPIHPGLETD
STKKGHRREHITYERDGYIVDESWCNPVCSRIRVIPRRELCVCKTCPKVCKLCRDDIQCMRPDPFCREIFRS

Therefore, preferably the RNA construct comprises a nucleotide sequence which encodes an amino acid sequence substantially as set out in SEQ ID No: 107, or a variant or fragment thereof.

In one embodiment, the SeV V polypeptide is encoded by the DNA nucleotide sequence of SEQ ID No: 108, as follows:

[SEQ ID No: 108]
ATGGATCAAGATGCCTTCATTCTTAAAGAAGATTCTGAAGTTGAGAGGGAGGCGCCAGGAGGAAGAGAGTCGCTCTCG
GATGTTATCGGATTCCTCGATGCTGTCCTGTCGAGTGAACCAACTGACATCGGAGGGGACAGAAGCTGGCTCCACAAC
ACCATCAACACTCCCCAAGGACCAGGCTCTGCCCATAGAGCCAAAAGTGAGGGCGAAGGAGAAGTCTCAACACCGTCG
ACCCAAGATAATCGATCAGGTGAGGAGAGTAGAGTCTCTGGGAGAACAAGCAAGCCAGAGGCAGAAGCACATGCTGGA
AACCTTGATAAACAAAATATACACCGGGCCTTTGGGGGAAGAACTGGTACAAACTCTGTATCTCAGGATCTGGGCGAT
GGAGGAGACTCCGGAATCCTTGAAAATCCCCCAAATGAGAGAGGATATCCGAGATCAGGTATTGAAGATGAAAACAGA
GAGATGGCTGCGCACCCTGATAAGAGGGGAGAAGACCAAGCTGAAGGACTTCCAGAAGAGGTACGAGGAGGTACATCC
CTACCTGATGAAGGAGAAGGTGGAGCAAGTAATAATGGAAGAAGCATGGAGCCTGGCAGCTCACATAGTGCAAGAGTA
ACTGGGGTCCTGGTGATTCCTAGCCCCGAACTCGAAGAGGCTGTGCTACGGAGGAACAAAAGAAGACCTACCAACAGT
GGGTCCAAACCTCTTACTCCAGCAACCGTGCCTGGCACCCGGTCCCCACCGCTGAATCGTTACAACAGCACAGGGTCA
CCACCAGGAAAACCCCCATCTACACAGGATGAGCACATCAACTCTGGGGACACCCCCGCCGTCAGGGTCAAAGACCGG
AAACCACCAATAGGGACCCGCTCTGTCTCAGATTGTCCAGCCAACGGCCGCCCAATCCACCCGGGTCTAGAGACCGAC
TCAACAAAAAAGGGgCATAGGAGAGAACACATCATCTATGAAAGAGATGGCTACATTGTTGACGAGTCTTGGTGTAAT
CCAGTCTGCTCAAGAATTCGAGTCATCCCGAGACGCGAGTTATGTGTTTGCAAGACGTGCCCTAAAGTCTGCAAACTA
TGCAGAGATGACATTCAATGTATGCGGCCTGATCCTTTCTGCCGAGAAATCTTCCGCTCG

Accordingly, preferably the SeV V polypeptide is encoded by the DNA nucleotide sequence substantially as set out in SEQ ID NO: 108, or a variant or fragment thereof.

One embodiment of codon optimised nucleic acid (DNA) encoding the SeV V polypeptide is provided herein as SEQ ID No: 109, as follows:

[SEQ ID No: 109]
ATGGACCAGGACGCCTTCATCCTGAAAGAGGACAGCGAGGTCGAGAGAGAAGCCCCTGGCGGAAGAGAAAGCCTGTCC
GATGTGATCGGCTTCCTGGATGCCGTGCTGAGCAGCGAGCCTACAGATATCGGCGGCGATAGAAGCTGGCTGCACAAC
ACCATCAACACCCCTCAAGGCCCTGGCTCTGCCCACAGAGCTAAGTCTGAAGGCGAGGGCGAAGTGTCTACCCCTAGC
ACACAGGACAACAGAAGCGGCGAGGAATCCAGAGTGTCCGGCAGAACAAGCAAGCCTGAGGCCGAAGCTCACGCCGGC
AATCTGGACAAGCAGAACATCCACAGAGCCTTCGGCGGCAGAACCGGCACAAATAGCGTGTCACAGGACCTCGGAGAT
GGCGGCGATTCTGGCATCCTGGAAAACCCTCCAAACGAGCGGGGCTACCCTAGAAGCGGAATCGAGGACGAGAACAGA
GAGATGGCCGCTCATCCCGACAAGAGAGGCGAAGATCAGGCCGAGGGACTGCCTGAAGAAGTGCGCGGAGGAACAAGC
CTGCCTGACGAAGGCGAAGGCGGAGCCTCTAACAACGGCAGATCTATGGAACCCGGCAGCAGCCATAGCGCCAGAGTT
ACAGGCGTGCTGGTCATCCCATCTCCAGAGCTGGAAGAGGCTGTGCTGAGGCGGAACAAGAGAAGGCCTACCAACAGC
GGCAGCAAGCCTCTGACACCAGCTACAGTGCCTGGCACAAGAAGCCCTCCACTGAACCGGTACAACAGCACAGGCTCT
CCACCTGGCAAGCCTCCATCCACACAGGATGAGCACATCAACTCCGGCGATACCCCTGCCGTCAGAGTGAAGGACAGA
AAGCCTCCTATCGGCACCAGAAGCGTGTCCGACTGTCCTGCCAATGGCAGACCTATTCACCCCGGCCTGGAAACCGAC
AGCACCAAGAAGGGACACAGACGGGAACACATCATCTACGAGCGCGACGGCTACATCGTGGACGAGAGCTGGTGCAAC
CCTGTGTGCAGCCGGATCAGAGTGATCCCTCGGAGAGAACTGTGCGTGTGCAAGACATGCCCCAAAGTGTGCAAGCTG
TGCCGGGACGACATCCAGTGTATGCGGCCCGATCCTTTCTGCAGAGAGATCTTCAGAAGC

Hence, preferably the RNA construct is encoded by a DNA sequence substantially as set out in SEQ ID No: 109, or a fragment or variant thereof.

In an embodiment, the RNA sequence corresponding to the codon optimised DNA sequence of SEQ ID No: 109 is provided herein as SEQ ID No: 110, as follows:

[SEQ ID No: 110]
AUGGACCAGGACGCCUUCAUCCUGAAAGAGGACAGCGAGGUCGAGAGAGAAGCCCCUGGCGGAAGAGAAAGCCUGUCC
GAUGUGAUCGGCUUCCUGGAUGCCGUGCUGAGCAGCGAGCCUACAGAUAUCGGCGGCGAUAGAAGCUGGCUGCACAAC
ACCAUCAACACCCCUCAAGGCCCUGGCUCUGCCCACAGAGCUAAGUCUGAAGGCGAGGGCGAAGUGUCUACCCCUAGC
ACACAGGACAACAGAAGCGGCGAGGAAUCCAGAGUGUCCGGCAGAACAAGCAAGCCUGAGGCCGAAGCUCACGCCGGC
AAUCUGGACAAGCAGAACAUCCACAGAGCCUUCGGCGGCAGAACCGGCACAAAUAGCGUGUCACAGGACCUCGGAGAU
GGCGGCGAUUCUGGCAUCCUGGAAAACCCUCCAAACGAGCGGGGCUACCCUAGAAGCGGAAUCGAGGACGAGAACAGA
GAGAUGGCCGCUCAUCCCGACAAGAGAGGCGAAGAUCAGGCCGAGGGACUGCCUGAAGAAGUGCGCGGAGGAACAAGC
CUGCCUGACGAAGGCGAAGGCGGAGCCUCUAACAACGGCAGAUCUAUGGAACCCGGCAGCAGCCAUAGCGCCAGAGUU
ACAGGCGUGCUGGUCAUCCCAUCUCCAGAGCUGGAAGAGGCUGUGCUGAGGCGGAACAAGAGAAGGCCUACCAACAGC
GGCAGCAAGCCUCUGACACCAGCUACAGUGCCUGGCACAAGAAGCCCUCCACUGAACCGGUACAACAGCACAGGCUCU
CCACCUGGCAAGCCUCCAUCCACACAGGAUGAGCACAUCAACUCCGGCGAUACCCCUGCCGUCAGAGUGAAGGACAGA
AAGCCUCCUAUCGGCACCAGAAGCGUGUCCGACUGUCCUGCCAAUGGCAGACCUAUUCACCCCGGCCUGGAAACCGAC
AGCACCAAGAAGGGACACAGACGGGAACACAUCAUCUACGAGCGCGACGGCUACAUCGUGGACGAGAGCUGGUGCAAC
CCUGUGUGCAGCCGGAUCAGAGUGAUCCCUCGGAGAGAACUGUGCGUGUGCAAGACAUGCCCCAAAGUGUGCAAGCUG
UGCCGGGACGACAUCCAGUGUAUGCGGCCCGAUCCUUUCUGCAGAGAGAUCUUCAGAAGC

Hence, preferably the RNA construct comprises a sequence substantially as set out in SEQ ID No: 110, or a fragment or variant thereof.

In one embodiment, the at least one IIP is Measles Non-structural protein V (strain Ichinose-B95a) (POC774), or an orthologue thereof. One embodiment of the polypeptide sequence of Measles V protein is represented herein as SEQ ID No: 111, as follows:

[SEQ ID No: 111]
MAEEQARHVKNGLECIRALKAEPIGSLAVEEAMAAWSEISDNPGQDRATCKEEEAGSSGLSKPCLSAIGSTEGGAPRI
RGQGSGESDDDAETLGIPSRNLQASSTGLQCYHVYDHSGEAVKGIQDADSIMVQSGLDGDSTLSGGDDESENSDVDIG
EPDTEGYAITDRGSAPISMGFRASDVETAEGGEIHELLKLQSRGNNFPKLGKTLNVPPPPNPSRASTSETPIKKGHRR
EIGLIWNGDRVFIDRWCNPMCSKVTLGTIRARCTCGECPRVCEQCRTDTGVDTRIWYHNLPEIPE

Therefore, preferably the RNA construct comprises a nucleotide sequence which encodes an amino acid sequence substantially as set out in SEQ ID No: 111, or a variant or fragment thereof.

In one embodiment, the Measles V polypeptide is encoded by the DNA nucleotide sequence of SEQ ID No: 112, as follows:

[SEQ ID No: 112]
ATGGCAGAAGAGCAGGCACGCCATGTCAAAAACGGACTGGAATGCATCCGGGCTCTCAAGGCCGAGCCCATCGGCTCA
CTGGCCGTCGAGGAAGCCATGGCAGCATGGTCAGAAATATCAGACAACCCAGGACAGGACCGAGCCACCTGCAAGGAA
GAGGAGGCAGGCAGTTCGGGTCTCAGCAAACCATGCCTCTCAGCAATTGGATCAACTGAAGGCGGTGCACCTCGCATC
CGCGGTCAGGGATCTGGAGAAAGCGATGACGACGCTGAAACTTTGGGAATCCCCTCAAGAAATCTCCAGGCATCAAGC
ACTGGGTTACAGTGTTATCATGTTTATGATCACAGCGGTGAAGCGGTTAAGGGAATCCAAGATGCTGACTCTATCATG
GTTCAATCAGGCCTTGATGGTGATAGCACCCTCTCAGGAGGAGACGATGAATCTGAAAACAGCGATGTGGATATTGGC
GAACCTGATACCGAGGGATATGCTATCACTGACCGGGGATCTGCTCCCATCTCTATGGGGTTCAGGGCTTCTGATGTT
GAAACTGCAGAAGGAGGGGAGATCCACGAGCTCCTGAAACTCCAATCCAGAGGCAACAACTTTCCGAAGCTTGGGAAA
ACTCTCAATGTTCCTCCGCCCCCGAACCCCAGTAGGGCCAGCACTTCCGAGACACCCATTAAAAAGGGgCACAGACGC
GAGATTGGCCTCATTTGGAACGGAGATCGCGTCTTTATTGACAGGTGGTGCAACCCAATGTGCTCGAAAGTCACCCTC
GGAACCATCAGGGCCAGGTGCACCTGCGGGGAATGTCCCCGAGIGTGTGAGCAATGCCGCACTGATACAGGAGTGGAC
ACCCGAATCTGGTACCACAATCTCCCCGAGATCCCAGAA

Accordingly, preferably the Measles V polypeptide is encoded by the DNA nucleotide sequence substantially as set out in SEQ ID NO: 112, or a variant or fragment thereof.

One embodiment of codon optimised nucleic acid (DNA) encoding the Measles V polypeptide is provided herein as SEQ ID No: 113, as follows:

[SEQ ID No: 113]
ATGGCCGAAGAACAGGCCAGACACGTGAAGAACGGCCTGGAATGCATCAGAGCCCTGAAGGCCGAGCCTATCGGATCT
CTGGCTGTGGAAGAAGCCATGGCCGCTTGGAGCGAGATCAGCGATAATCCCGGCCAGGACCGGGCCACCTGTAAAGAA
GAAGAGGCCGGATCTAGCGGCCTGAGCAAGCCTTGTCTGTCTGCCATCGGCTCTACAGAAGGCGGCGCTCCTAGAATC
AGAGGCCAAGGATCTGGCGAGAGCGACGACGATGCTGAGACACTGGGCATCCCCAGCAGAAATCTGCAGGCCAGCTCT
ACCGGCCTGCAGTGCTATCACGTGTACGATCACTCTGGCGAGGCCGTGAAGGGAATCCAGGATGCCGATAGCATCATG
GTGCAGAGCGGCCTGGATGGCGACTCTACACTTAGCGGCGGAGATGACGAGAGCGAGAACTCCGATGTGGACATCGGC
GAGCCTGATACAGAGGGCTACGCCATCACAGACAGAGGCAGCGCCCCTATCAGCATGGGCTTTAGAGCCAGCGACGTG
GAAACAGCCGAAGGCGGAGAGATTCACGAGCTGCTGAAGCTGCAGAGCCGGGGCAACAACTTTCCCAAGCTGGGCAAG
ACCCTGAACGTGCCACCTCCTCCAAATCCTAGCAGAGCCAGCACCAGCGAGACACCCATCAAGAAGGGCCACAGAAGA
GAGATCGGCCTGATCTGGAACGGCGACCGGGTGTTCATCGACAGATGGTGCAACCCCATGTGCAGCAAAGTGACCCTG
GGCACCATCCGGGCCAGATGTACATGCGGAGAGTGCCCTAGAGTGTGCGAGCAGTGCAGAACCGATACCGGCGTGGAC
ACCCGGATCTGGTATCACAACCTGCCTGAGATCCCCGAG

Hence, preferably the RNA construct is encoded by a DNA sequence substantially as set out in SEQ ID No: 113, or a fragment or variant thereof.

In an embodiment, the RNA sequence corresponding to the codon optimised DNA sequence of SEQ ID No: 113 is provided herein as SEQ ID No: 114, as follows:

[SEQ ID No: 114]
AUGGCCGAAGAACAGGCCAGACACGUGAAGAACGGCCUGGAAUGCAUCAGAGCCCUGAAGGCCGAGCCUAUCGGAUCU
CUGGCUGUGGAAGAAGCCAUGGCCGCUUGGAGCGAGAUCAGCGAUAAUCCCGGCCAGGACCGGGCCACCUGUAAAGAA
GAAGAGGCCGGAUCUAGCGGCCUGAGCAAGCCUUGUCUGUCUGCCAUCGGCUCUACAGAAGGCGGCGCUCCUAGAAUC
AGAGGCCAAGGAUCUGGCGAGAGCGACGACGAUGCUGAGACACUGGGCAUCCCCAGCAGAAAUCUGCAGGCCAGCUCU
ACCGGCCUGCAGUGCUAUCACGUGUACGAUCACUCUGGCGAGGCCGUGAAGGGAAUCCAGGAUGCCGAUAGCAUCAUG
GUGCAGAGCGGCCUGGAUGGCGACUCUACACUUAGCGGCGGAGAUGACGAGAGCGAGAACUCCGAUGUGGACAUCGGC
GAGCCUGAUACAGAGGGCUACGCCAUCACAGACAGAGGCAGCGCCCCUAUCAGCAUGGGCUUUAGAGCCAGCGACGUG
GAAACAGCCGAAGGCGGAGAGAUUCACGAGCUGCUGAAGCUGCAGAGCCGGGGCAACAACUUUCCCAAGCUGGGCAAG
ACCCUGAACGUGCCACCUCCUCCAAAUCCUAGCAGAGCCAGCACCAGCGAGACACCCAUCAAGAAGGGCCACAGAAGA
GAGAUCGGCCUGAUCUGGAACGGCGACCGGGUGUUCAUCGACAGAUGGUGCAACCCCAUGUGCAGCAAAGUGACCCUG
GGCACCAUCCGGGCCAGAUGUACAUGCGGAGAGUGCCCUAGAGUGUGCGAGCAGUGCAGAACCGAUACCGGCGUGGAC
ACCCGGAUCUGGUAUCACAACCUGCCUGAGAUCCCCGAG

Hence, preferably the RNA construct comprises a sequence substantially as set out in SEQ ID No: 114, or a fragment or variant thereof.

In one embodiment, the at least one IIP is Nipah virus Non-structural protein V (NiV V protein) (Q997F2; V_NIPAV), or an orthologue thereof. One embodiment of the polypeptide sequence of NiV V protein is represented herein as SEQ ID No: 115, as follows:

[SEQ ID No: 115]
MDKLELVNDGLNIIDFIQKNQKEIQKTYGRSSIQQPSIKDQTKAWEDFLQCTSGESEQVEGGMSKDDGDVERRNLEDL
SSTSPTDGTIGKRVSNTRDWAEGSDDIQLDPVVTDVVYHDHGGECTGYGFTSSPERGWSDYTSGANNGNVCLVSDAKM
LSYAPEIAVSKEDRETDLVHLENKLSTTGLNPTAVPFTLRNLSDPAKDSPVIAEHYYGLGVKEQNVGPQTSRNVNLDS
IKLYTSDDEEADQLEFEDEFAGSSSEVIVGISPEDEEPSSVGGKPNESIGRTIEGQSIRDNLQAKDNKSTDVPGAGPK
DSAVKEEPPQKRLPMLAEEFECSGSEDPIIRELLKENSLINCQQGKDAQPPYHWSIERSISPDKTEIVNGAVQTADRQ
RPGTPMPKSRGIPIKKGHRREISICWDGKRAWVEEWCNPACSRITPLPRRQECQCGECPTECFHCG

Therefore, preferably the RNA construct comprises a nucleotide sequence which encodes an amino acid sequence substantially as set out in SEQ ID No: 115, or a variant or fragment thereof.

In one embodiment, the NiV V polypeptide is encoded by the DNA nucleotide sequence of SEQ ID No: 116, as follows:

[SEQ ID No: 116]
ATGGATAAATTGGAACTAGTCAATGATGGCCTCAATATTATTGACTTTATTCAGAAGAACCAAAAAGAAATACAGAAG
ACATACGGACGATCAAGTATTCAACAACCCAGCATCAAAGATCAAACAAAAGCCTGGGAAGATTTTCTGCAGTGCACC
AGTGGAGAATCTGAACAAGTTGAGGGGGGAATGTCTAAGGATGATGGAGATGTTGAAAGAAGAAACTTGGAGGATCTA
TCCAGTACTTCTCCCACAGATGGAACTATTGGAAAGAGAGTGTCGAACACCCGTGACTGGGCAGAAGGTTCAGATGAC
ATACAACTGGACCCAGTGGTTACAGACGTTGTATACCATGATCATGGAGGAGAATGTACCGGATATGGATTTACTTCA
AGCCCTGAGAGAGGGTGGAGTGATTACACATCAGGAGCAAACAATGGGAATGTATGTCTTGTATCTGATGCAAAGATG
CTGTCCTATGCTCCCGAAATTGCAGTTTCTAAAGAAGATCGGGAAACTGATCTAGTTCATCTTGAGAATAAACTATCT
ACTACAGGACTGAATCCCACAGCAGTACCGTTCACTCTGAGAAACCTGTCTGATCCTGCAAAAGACTCTCCTGTGATT
GCTGAACACTACTACGGACTAGGAGTTAAAGAGCAAAACGTTGGCCCTCAGACTAGCAGAAATGTCAATTTGGACAGC
ATCAAATTGTACACATCAGATGACGAAGAGGCAGATCAGCTTGAATTCGAAGATGAGTTTGCAGGAAGCTCAAGTGAA
GTGATAGTCGGCATTTCTCCTGAAGATGAAGAGCCTTCAAGTGTTGGCGGAAAACCCAATGAATCCATTGGACGTACA
ATCGAAGGCCAATCAATCCGAGACAACCTTCAAGCCAAGGACAACAAATCAACAGATGTACCAGGAGCAGGACCGAAA
GATTCAGCAGTGAAGGAAGAACCACCCCAGAAGAGGCTACCTATGTTAGCTGAAGAATTTGAGTGCTCTGGATCGGAA
GACCCAATCATTCGGGAGCTGCTGAAGGAGAACTCACTCATAAATTGTCAGCAAGGGAAAGATGCTCAGCCTCCATAT
CATTGGAGCATCGAGAGGTCAATAAGCCCGGATAAAACTGAGATCGTCAACGGTGCTGTGCAAACTGCTGACAGGCAA
AGACCAGGAACTCCGATGCCAAAGTCCCGAGGTATTCCCATTAAAAAGGGGCACAGACGCGAAATATCCATCTGCTGG
GACGGAAAACGTGCCTGGGTCGAAGAGTGGTGCAACCCGGCATGTTCGAGGATCACCCCCCTACCAAGAAGGCAAGAG
TGTCAATGCGGAGAATGTCCAACTGAATGCTTCCACTGCGGT

Accordingly, preferably the NiV V polypeptide is encoded by the DNA nucleotide sequence substantially as set out in SEQ ID NO: 116, or a variant or fragment thereof.

One embodiment of codon optimised nucleic acid (DNA) encoding the NiV V polypeptide is provided herein as SEQ ID No: 117, as follows:

[SEQ ID No: 117]
ATGGACAAGCTGGAACTGGTCAACGACGGCCTGAACATCATCGACTTCATCCAGAAGAACCAGAAAGAGATCCAGAAA
ACCTACGGCCGGTCCAGCATCCAGCAGCCTAGCATCAAGGATCAGACCAAGGCCTGGGAAGATTTCCTGCAGTGTACC
AGCGGCGAGAGCGAACAGGTTGAAGGCGGCATGAGCAAGGACGACGGCGACGTGGAAAGACGGAACCTGGAAGATCTG
AGCAGCACAAGCCCTACCGATGGCACCATCGGCAAGCGGGTGTCCAACACAAGAGATTGGGCCGAGGGCAGCGACGAC
ATTCAGCTGGATCCTGTGGTCACCGATGTGGTGTACCACGATCACGGCGGCGAGTGTACAGGCTACGGCTTTACAAGC
AGCCCCGAGAGAGGCTGGAGCGATTATACAAGCGGCGCCAACAACGGCAACGTGTGCCTGGTGTCTGACGCCAAGATG
CTGAGCTACGCCCCTGAGATCGCCGTGTCCAAAGAGGACAGAGAAACCGACCTGGTGCACCTGGAAAACAAGCTGAGC
ACCACCGGACTGAACCCTACCGCCGTGCCTTTCACACTGAGAAACCTGAGCGACCCCGCCAAGGACTCTCCTGTGATT
GCCGAGCACTACTACGGCCTGGGCGTGAAAGAACAGAACGTGGGCCCTCAGACCAGCCGGAACGTGAACCTGGATTCC
ATCAAGCTGTACACCTCCGACGACGAGGAAGCCGACCAGCTGGAATTCGAGGATGAGTTTGCCGGCAGCAGCAGCGAA
GTGATTGTGGGCATCAGCCCTGAGGACGAGGAACCTAGCTCTGTTGGCGGCAAGCCCAATGAGAGCATCGGCAGAACA
ATCGAGGGCCAGAGCATCCGGGATAACCTGCAGGCCAAGGACAACAAGAGCACCGATGTTCCAGGCGCTGGCCCTAAG
GATAGCGCCGTGAAAGAGGAACCACCTCAGAAACGGCTGCCCATGCTGGCCGAGGAATTTGAGTGTAGCGGCAGCGAG
GACCCCATCATCAGAGAGCTGCTGAAAGAGAACAGCCTGATCAACTGCCAGCAGGGCAAAGACGCCCAGCCTCCTTAC
CACTGGTCCATCGAGAGATCTATCAGCCCCGACAAGACCGAGATCGTGAATGGCGCTGTGCAGACCGCCGATAGACAG
AGGCCTGGAACTCCCATGCCTAAGAGCAGAGGCATCCCCATCAAGAAGGGCCACAGAAGAGAGATCAGCATCTGCTGG
GACGGCAAGCGCGCCTGGGTTGAAGAGTGGTGTAATCCCGCCTGCAGCCGGATCACACCTCTGCCTAGAAGGCAAGAG
TGCCAGTGTGGCGAGTGTCCCACCGAGTGTTTTCACTGTGGA

Hence, preferably the RNA construct is encoded by a DNA sequence substantially as set out in SEQ ID No: 117, or a fragment or variant thereof.

In an embodiment, the RNA sequence corresponding to the codon optimised DNA sequence of SEQ ID No: 117 is provided herein as SEQ ID No: 118, as follows:

[SEQ ID No: 118]
AUGGACAAGCUGGAACUGGUCAACGACGGCCUGAACAUCAUCGACUUCAUCCAGAAGAACCAGAAAGAGAUCCAGAAA
ACCUACGGCCGGUCCAGCAUCCAGCAGCCUAGCAUCAAGGAUCAGACCAAGGCCUGGGAAGAUUUCCUGCAGUGUACC
AGCGGCGAGAGCGAACAGGUUGAAGGCGGCAUGAGCAAGGACGACGGCGACGUGGAAAGACGGAACCUGGAAGAUCUG
AGCAGCACAAGCCCUACCGAUGGCACCAUCGGCAAGCGGGUGUCCAACACAAGAGAUUGGGCCGAGGGCAGCGACGAC
AUUCAGCUGGAUCCUGUGGUCACCGAUGUGGUGUACCACGAUCACGGCGGCGAGUGUACAGGCUACGGCUUUACAAGC
AGCCCCGAGAGAGGCUGGAGCGAUUAUACAAGCGGCGCCAACAACGGCAACGUGUGCCUGGUGUCUGACGCCAAGAUG
CUGAGCUACGCCCCUGAGAUCGCCGUGUCCAAAGAGGACAGAGAAACCGACCUGGUGCACCUGGAAAACAAGCUGAGC
ACCACCGGACUGAACCCUACCGCCGUGCCUUUCACACUGAGAAACCUGAGCGACCCCGCCAAGGACUCUCCUGUGAUU
GCCGAGCACUACUACGGCCUGGGCGUGAAAGAACAGAACGUGGGCCCUCAGACCAGCCGGAACGUGAACCUGGAUUCC
AUCAAGCUGUACACCUCCGACGACGAGGAAGCCGACCAGCUGGAAUUCGAGGAUGAGUUUGCCGGCAGCAGCAGCGAA
GUGAUUGUGGGCAUCAGCCCUGAGGACGAGGAACCUAGCUCUGUUGGCGGCAAGCCCAAUGAGAGCAUCGGCAGAACA
AUCGAGGGCCAGAGCAUCCGGGAUAACCUGCAGGCCAAGGACAACAAGAGCACCGAUGUUCCAGGCGCUGGCCCUAAG
GAUAGCGCCGUGAAAGAGGAACCACCUCAGAAACGGCUGCCCAUGCUGGCCGAGGAAUUUGAGUGUAGCGGCAGCGAG
GACCCCAUCAUCAGAGAGCUGCUGAAAGAGAACAGCCUGAUCAACUGCCAGCAGGGCAAAGACGCCCAGCCUCCUUAC
CACUGGUCCAUCGAGAGAUCUAUCAGCCCCGACAAGACCGAGAUCGUGAAUGGCGCUGUGCAGACCGCCGAUAGACAG
AGGCCUGGAACUCCCAUGCCUAAGAGCAGAGGCAUCCCCAUCAAGAAGGGCCACAGAAGAGAGAUCAGCAUCUGCUGG
GACGGCAAGCGCGCCUGGGUUGAAGAGUGGUGUAAUCCCGCCUGCAGCCGGAUCACACCUCUGCCUAGAAGGCAAGAG
UGCCAGUGUGGCGAGUGUCCCACCGAGUGUUUUCACUGUGGA

Hence, preferably the RNA construct comprises a sequence substantially as set out in SEQ ID No: 118, or a fragment or variant thereof.

In one embodiment, the at least one IIP is POLG_EMCV (Encephalomyocarditis virus 2C) genome polyprotein (Li L, Fan H, Song Z, Liu X, Bai J, Jiang P (2019) Encephalomyocarditis virus 2C protein antagonizes interferon-B signaling pathway through interaction with MDA5 Antiviral Res, 161, 70-84), or an orthologue thereof. One embodiment of the polypeptide sequence of EMCV is represented herein as SEQ ID No: 119, as follows:

[SEQ ID No: 119]
LKARDINDIFAILKNGEWLVKLILAIRDWIKAWIASEEKFVTMTDLVPGILEKQRDLNDPSKYKEAKEWLDNARQACL
KSGNVHIANLCKVVAPAPSKSRPEPVVVCLRGKSGQGKSFLANVLAQAISTHFTGRIDSVWYCPPDPDHFDGYNQQTV
VVMDDLGQNPDGKDFKYFAQMVSTTGFIPPMASLEDKGKPFNSKVIIATTNLYSGFTPRTMVCPDALNRRFHFDIDVS
AKDGYKINSKLDIIKALEDTHANPVAMFQYDCALLNGMAVEMKRMQQDMFKPQPPLQNVYQLVQEVIDRVELHEKVSS
HPIFKQ

Therefore, preferably the RNA construct comprises a nucleotide sequence which encodes an amino acid sequence substantially as set out in SEQ ID No: 119, or a variant or fragment thereof.

In one embodiment, the EMCV polypeptide is encoded by the DNA nucleotide sequence of SEQ ID No: 120, as follows:

[SEQ ID No: 120]
CTCAAAGCACGTGACATCAACGACATCTTCGCCATTCTCAAGAACGGCGAGTGGCTGGTCAAACTGATCCTTGCCATC
CGCGACTGGATTAAGGCTTGGATCGCCTCAGAAGAGAAGTTTGTCACCATGACAGACTTGGTGCCTGGCATCCTTGAA
AAGCAGCGGGACCTGAACGACCCGAGCAAGTACAAGGAAGCCAAGGAGTGGCTCGACAACGCGCGCCAAGCGTGTTTG
AAGAGCGGGAACGTCCACATTGCCAACCTGTGCAAAGTGGTCGCACCAGCACCCAGCAAGTCGAGGCCCGAACCCGTG
GTTGTTTGCCTCCGCGGCAAATCTGGCCAGGGCAAGAGCTTCCTTGCAAACGTGCTTGCACAGGCAATTTCCACCCAC
TTCACCGGCAGAATCGACTCAGTGTGGTACTGCCCACCTGACCCTGACCACTTCGACGGTTACAACCAGCAAACCGTT
GTTGTGATGGATGATTTGGGCCAGAACCCTGACGGCAAGGACTTCAAATACTTTGCCCAAATGGTCTCGACCACAGGG
TTTATCCCGCCCATGGCATCACTCGAGGACAAAGGTAAACCTTTCAACAGCAAAGTCATCATCGCGACCACCAACTTG
TACTCGGGCTTCACCCCGAGGACCATGGTATGTCCCGACGCACTGAACCGGAGGTTTCACTTTGACATCGATGTGAGT
GCTAAGGATGGGTACAAAATTAACAGCAAATTGGACATTATCAAAGCACTCGAAGACACCCACGCCAACCCAGTGGCA
ATGTTTCAATACGACTGIGCCCTTCTCAACGGCATGGCCGTTGAAATGAAGAGAATGCAACAAGACATGTTCAAGCCT
CAACCACCCCTCCAGAATGTGTACCAGCTTGTTCAGGAGGTGATCGATCGGGTCGAGCTCCACGAGAAAGTGTCGAGT
CACCCGATCTTCAAGCAG

Accordingly, preferably the EMCV polypeptide is encoded by the DNA nucleotide sequence substantially as set out in SEQ ID NO: 120, or a variant or fragment thereof.

One embodiment of codon optimised nucleic acid (DNA) encoding the EMCV polypeptide is provided herein as SEQ ID No: 121, as follows:

[SEQ ID No: 121]
CTGAAGGCCAGAGACATCAACGACATCTTCGCCATCCTGAAGAACGGCGAGTGGCTGGTCAAGCTGATCCTGGCCATC
AGAGACTGGATCAAGGCCTGGATCGCCAGCGAAGAGAAGTTCGTGACCATGACCGATCTGGTGCCCGGCATCCTGGAA
AAGCAGAGGGACCTGAACGACCCCAGCAAGTACAAAGAGGCCAAAGAATGGCTGGACAACGCCAGACAGGCCTGCCTG
AAGTCCGGCAATGTGCATATCGCCAACCTGTGCAAGGTGGTGGCCCCTGCTCCTAGCAAGTCTAGACCTGAGCCTGTG
GTCGTGTGCCTGAGAGGCAAATCTGGCCAGGGCAAGAGCTTCCTGGCCAATGTTCTGGCCCAGGCCATCAGCACCCAC
TTCACCGGAAGAATCGACAGCGTGTGGTACTGCCCTCCTGATCCTGACCACTTCGACGGCTACAACCAGCAGACCGTG
GTGGTCATGGACGACCTGGGACAGAACCCCGACGGCAAGGACTTCAAGTACTTCGCCCAGATGGTGTCCACCACCGGC
TTCATTCCTCCAATGGCCAGCCTGGAAGATAAGGGCAAGCCCTTCAACAGCAAAGTGATCATTGCCACCACCAACCTG
TACAGCGGCTTCACCCCTAGAACCATGGTCTGCCCCGACGCTCTGAACAGACGGTTCCACTTTGACATCGACGTGTCC
GCCAAGGATGGCTACAAGATCAACTCCAAGCTGGACATCATCAAGGCCCTCGAGGACACCCACGCCAATCCTGTGGCC
ATGTTCCAGTACGATTGCGCCCTGCTGAATGGCATGGCCGTGGAAATGAAGCGGATGCAGCAGGACATGTTCAAGCCC
CAGCCTCCACTGCAGAACGTGTACCAGCTCGTGCAAGAAGTGATCGACCGGGTCGAGCTGCACGAGAAGGTGTCCTCT
CATCCCATCTTCAAGCAG

Hence, preferably the RNA construct is encoded by a DNA sequence substantially as set out in SEQ ID No: 121, or a fragment or variant thereof.

In an embodiment, the RNA sequence corresponding to the codon optimised DNA sequence of SEQ ID No: 121 is provided herein as SEQ ID No: 122, as follows:

[SEQ ID No: 122]
CUGAAGGCCAGAGACAUCAACGACAUCUUCGCCAUCCUGAAGAACGGCGAGUGGCUGGUCAAGCUGAUCCUGGCCAUC
AGAGACUGGAUCAAGGCCUGGAUCGCCAGCGAAGAGAAGUUCGUGACCAUGACCGAUCUGGUGCCCGGCAUCCUGGAA
AAGCAGAGGGACCUGAACGACCCCAGCAAGUACAAAGAGGCCAAAGAAUGGCUGGACAACGCCAGACAGGCCUGCCUG
AAGUCCGGCAAUGUGCAUAUCGCCAACCUGUGCAAGGUGGUGGCCCCUGCUCCUAGCAAGUCUAGACCUGAGCCUGUG
GUCGUGUGCCUGAGAGGCAAAUCUGGCCAGGGCAAGAGCUUCCUGGCCAAUGUUCUGGCCCAGGCCAUCAGCACCCAC
UUCACCGGAAGAAUCGACAGCGUGUGGUACUGCCCUCCUGAUCCUGACCACUUCGACGGCUACAACCAGCAGACCGUG
GUGGUCAUGGACGACCUGGGACAGAACCCCGACGGCAAGGACUUCAAGUACUUCGCCCAGAUGGUGUCCACCACCGGC
UUCAUUCCUCCAAUGGCCAGCCUGGAAGAUAAGGGCAAGCCCUUCAACAGCAAAGUGAUCAUUGCCACCACCAACCUG
UACAGCGGCUUCACCCCUAGAACCAUGGUCUGCCCCGACGCUCUGAACAGACGGUUCCACUUUGACAUCGACGUGUCC
GCCAAGGAUGGCUACAAGAUCAACUCCAAGCUGGACAUCAUCAAGGCCCUCGAGGACACCCACGCCAAUCCUGUGGCC
AUGUUCCAGUACGAUUGCGCCCUGCUGAAUGGCAUGGCCGUGGAAAUGAAGCGGAUGCAGCAGGACAUGUUCAAGCCC
CAGCCUCCACUGCAGAACGUGUACCAGCUCGUGCAAGAAGUGAUCGACCGGGUCGAGCUGCACGAGAAGGUGUCCUCU
CAUCCCAUCUUCAAGCAG

Hence, preferably the RNA construct comprises a sequence substantially as set out in SEQ ID No: 122, or a fragment or variant thereof.

In one embodiment, the at least one IIP is Birnavirus VP3 (Avian infectious bursal disease virus (IBDV) (Gumboro disease virus) Capsid Protein VP3), or an orthologue thereof (Ye C, Jia L, Sun Y, Hu B, Wang L, Xingmeng L, Zu J (2014) Inhibition of antiviral innate immunity by birnavirus VP3 protein via blockage of viral double-stranded RNA binding to the host cytoplasmic RNA detector MDA5. J Virol. 88, 18, 11154-11156). One embodiment of the polypeptide sequence of Birnavirus VP3 is represented herein as SEQ ID No: 123, as follows:

[SEQ ID No: 123]
ASEFKETPELESAVRAMEAAANVDPLFQSALSVFMWLEENGIVTDMANFALDSPNAHRMRNFLANAPQAGS
KSQRAKYGTAGYGVEARGPTPEEAQREKDTRISKKMETMGIYFATPEWVALNGHRGPSPGQLKYWQNTREIPDPNEDY
LDYVHAEKSRLASEEQILRAATSIYGAPGQAEPPQAFIDEVAKVYEINHGRGPNQEQMKDLLLTAMEMKHRNPRRALP
KPKPKPNAPTQRPPGRLGRWIRTVSDEDLE

Therefore, preferably the RNA construct comprises a nucleotide sequence which encodes an amino acid sequence substantially as set out in SEQ ID No: 123, or a variant or fragment thereof.

In one embodiment, the Birnavirus VP3 polypeptide is encoded by the DNA nucleotide sequence of SEQ ID No: 124, as follows:

[SEQ ID No: 124]
GCATCAGAGTTCAAAGAGACCCCCGAACTCGAGAGTGCCGTCAGAGCAATGGAAGCAGCAGCCAACGTGGACCCACTA
TTCCAATCTGCACTCAGTGTGTTCATGTGGCTGGAAGAGAATGGGATTGTGACTGACATGGCCAACTTCGCACTCAGC
GACCCGAACGCCCATCGGATGCGAAATTTTCTTGCAAACGCACCACAAGCAGGCAGCAAGTCGCAAAGGGCCAAGTAC
GGGACAGCAGGCTACGGAGTGGAGGCTCGGGGCCCCACACCAGAGGAAGCACAGAGGGAAAAAGACACACGGATCTCA
AAGAAGATGGAGACCATGGGCATCTACTTTGCAACACCAGAATGGGTAGCACTCAATGGGCACCGAGGGCCAAGCCCC
GGCCAGCTAAAGTACTGGCAGAACACACGAGAAATACCGGACCCAAACGAGGACTATCTAGACTACGTGCATGCAGAG
AAGAGCCGGTTGGCATCAGAAGAACAAATCCTAAGGGCAGCTACGTCGATCTACGGGGCTCCAGGACAGGCAGAGCCA
CCCCAAGCTTTCATAGACGAAGTTGCCAAAGTCTATGAAATCAACCATGGACGTGGCCCAAACCAAGAACAGATGAAA
GATCTGCTCTTGACTGCGATGGAGATGAAGCATCGCAATCCCAGGCGGGCTCTACCAAAGCCCAAGCCAAAACCCAAT
GCTCCAACACAGAGACCCCCTGGTCGGCTGGGCCGCTGGATCAGGACCGTCTCTGATGAGGACCTTGAG

Accordingly, preferably the Birnavirus VP3 polypeptide is encoded by the DNA nucleotide sequence substantially as set out in SEQ ID NO: 124, or a variant or fragment thereof.

One embodiment of codon optimised nucleic acid (DNA) encoding the Birnavirus VP3 polypeptide is provided herein as SEQ ID No: 125, as follows:

[SEQ ID No: 125]
GCCAGCGAGTTCAAAGAGACACCCGAGCTGGAAAGCGCCGTCAGAGCTATGGAAGCCGCCGCTAATGTGGACCCTCTG
TTTCAGTCTGCCCTGAGCGTGTTCATGTGGCTGGAAGAGAACGGCATCGTGACCGACATGGCCAACTTCGCCCTGTCT
GACCCCAACGCTCACCGGATGAGAAACTTTCTGGCCAACGCTCCTCAGGCCGGCAGCAAGTCTCAGAGAGCCAAATAC
GGCACAGCCGGCTACGGCGTGGAAGCCAGAGGACCTACACCTGAGGAAGCCCAGAGAGAGAAGGACACCCGGATCAGC
AAGAAAATGGAAACCATGGGCATCTACTTCGCCACACCTGAGTGGGTCGCCCTGAATGGACACAGAGGACCATCTCCA
GGCCAGCTGAAGTACTGGCAGAACACCAGAGAGATCCCCGATCCTAACGAGGACTACCTGGACTACGTGCACGCCGAG
AAAAGCAGACTGGCCAGCGAGGAACAGATCCTGAGAGCCGCCACATCCATCTATGGCGCTCCAGGACAAGCCGAACCT
CCACAGGCCTTTATCGACGAGGTGGCCAAGGTGTACGAGATCAACCACGGCAGAGGCCCCAATCAAGAGCAGATGAAG
GACCTGCTGCTGACCGCCATGGAAATGAAGCACAGAAACCCCAGACGGGCCCTGCCTAAGCCAAAGCCTAAACCTAAC
GCTCCCACACAGCGGCCTCCAGGCAGACTCGGAAGATGGATCAGAACCGTGTCCGACGAGGACCTGGAA

Hence, preferably the RNA construct is encoded by a DNA sequence substantially as set out in SEQ ID No: 125, or a fragment or variant thereof.

In an embodiment, the RNA sequence corresponding to the codon optimised DNA sequence of SEQ ID No: 125 is provided herein as SEQ ID No: 126, as follows:

[SEQ ID No: 126]
GCCAGCGAGUUCAAAGAGACACCCGAGCUGGAAAGCGCCGUCAGAGCUAUGGAAGCCGCCGCUAAUGUGGACCCUCUG
UUUCAGUCUGCCCUGAGCGUGUUCAUGUGGCUGGAAGAGAACGGCAUCGUGACCGACAUGGCCAACUUCGCCCUGUCU
GACCCCAACGCUCACCGGAUGAGAAACUUUCUGGCCAACGCUCCUCAGGCCGGCAGCAAGUCUCAGAGAGCCAAAUAC
GGCACAGCCGGCUACGGCGUGGAAGCCAGAGGACCUACACCUGAGGAAGCCCAGAGAGAGAAGGACACCCGGAUCAGC
AAGAAAAUGGAAACCAUGGGCAUCUACUUCGCCACACCUGAGUGGGUCGCCCUGAAUGGACACAGAGGACCAUCUCCA
GGCCAGCUGAAGUACUGGCAGAACACCAGAGAGAUCCCCGAUCCUAACGAGGACUACCUGGACUACGUGCACGCCGAG
AAAAGCAGACUGGCCAGCGAGGAACAGAUCCUGAGAGCCGCCACAUCCAUCUAUGGCGCUCCAGGACAAGCCGAACCU
CCACAGGCCUUUAUCGACGAGGUGGCCAAGGUGUACGAGAUCAACCACGGCAGAGGCCCCAAUCAAGAGCAGAUGAAG
GACCUGCUGCUGACCGCCAUGGAAAUGAAGCACAGAAACCCCAGACGGGCCCUGCCUAAGCCAAAGCCUAAACCUAAC
GCUCCCACACAGCGGCCUCCAGGCAGACUCGGAAGAUGGAUCAGAACCGUGUCCGACGAGGACCUGGAA

Hence, preferably the RNA construct comprises a sequence substantially as set out in SEQ ID No: 126, or a fragment or variant thereof.

In one embodiment, the at least one IIP is Porcine delta coronavirus NS6 protein (A0A0K2D1N4), or an orthologue thereof. Fang P, Fang L, Ren J, Hong Y, Liu X, Zhao Y, Wang D, Peng G, Xiai S (2018) Porcine deltacoronavirus accessory protein NS6 antagonises interferon beta production by interfering with the binding of RIG-I/MDA-5 to double stranded RNA. J Virol., 92, 15, e00712-18. One embodiment of the polypeptide sequence of Porcine delta coronavirus NS6 is represented herein as SEQ ID No: 127, as follows:

[SEQ ID No: 127]
MCNCHLQLRDLYRLCNKLHIRRDDVPELIDPLVKTRCFAYSLVVLANAPIAFSILPRKILINGEPLLLEYGSIYGKD
FIIRPSLQVILEDELN

Therefore, preferably the RNA construct comprises a nucleotide sequence which encodes an amino acid sequence substantially as set out in SEQ ID No: 127, or a variant or fragment thereof.

In one embodiment, the Porcine delta coronavirus NS6 polypeptide is encoded by the DNA nucleotide sequence of SEQ ID No: 128, as follows:

[SEQ ID No: 128]
ATGTGCAACTGCCATCTGCAGCTGCGAGATTTATATAGATTGTGCAATAAGCTGCACATCAGAAGAGACGATGTTCCT
GAGCTTATTGACCCTCTCGTTAAAACTCGCTGTTTTGCTTACAGTCTCGTGGTTCTTGCTAATGCTAATCCAATTGCA
TTTAGCATACTACCTCGGAAAATTCTTATCAATGGTGAGCCTTTACTGCTTGAATATGGTAGCATATATGGTAAAGAC
TTTATCATTAGACCATCGCTCCAAGTCATTCTTGAAGATGAATTAAAT

Accordingly, preferably the Porcine delta coronavirus NS6 polypeptide is encoded by the DNA nucleotide sequence substantially as set out in SEQ ID NO: 128, or a variant or fragment thereof.

One embodiment of codon optimised nucleic acid (DNA) encoding the Porcine delta coronavirus NS6 polypeptide is provided herein as SEQ ID No: 129, as follows:

[SEQ ID No: 129]
ATGTGCAACTGCCATCTGCAGCTGCGGGACCTGTACCGGCTGTGTAACAAGCTGCACATCAGACGGGACGACGTGCCC
GAGCTGATCGATCCTCTGGTCAAGACCAGATGCTTCGCCTACAGCCTGGTGGTGCTGGCCAACGCCAATCCTATCGCC
TTCAGCATCCTGCCTCGGAAGATCCTGATCAACGGCGAGCCTCTGCTGCTGGAATACGGCAGCATCTACGGCAAGGAC
TTCATCATCAGACCCAGCCTGCAAGTGATCCTGGAAGATGAGCTGAACTGA

Hence, preferably the RNA construct is encoded by a DNA sequence substantially as set out in SEQ ID No: 129, or a fragment or variant thereof.

In an embodiment, the RNA sequence corresponding to the codon optimised DNA sequence of SEQ ID No: 129 is provided herein as SEQ ID No: 130, as follows:

[SEQ ID No: 130]
AUGUGCAACUGCCAUCUGCAGCUGCGGGACCUGUACCGGCUGUGUAACAAGCUGCACAUCAGACGGGACGACGUGCCC
GAGCUGAUCGAUCCUCUGGUCAAGACCAGAUGCUUCGCCUACAGCCUGGUGGUGCUGGCCAACGCCAAUCCUAUCGCC
UUCAGCAUCCUGCCUCGGAAGAUCCUGAUCAACGGCGAGCCUCUGCUGCUGGAAUACGGCAGCAUCUACGGCAAGGAC
UUCAUCAUCAGACCCAGCCUGCAAGUGAUCCUGGAAGAUGAGCUGAACUGA

Hence, preferably the RNA construct comprises a sequence substantially as set out in SEQ ID No: 130, or a fragment or variant thereof.

In one embodiment, the at least one IIP is LASV Z protein (073557|RING finger protein Z Lassa virus (strain Mouse/Sierra Leone/Josiah/1976), or an orthologue thereof (Xing J, Ly H, Liang Y J (2015) The Z proteins of pathogenic but not nonpathogenic arenaviruses inhibit RIG-I-like receptor-dependent interferon production. J Virol., 89, 5, 2944-2955. One embodiment of the polypeptide sequence of LASV Z is represented herein as SEQ ID No: 131, as follows:

[SEQ ID No: 131]
MGNKQAKAPESKDSPRASLIPDATHLGPQFCKSCWFENKGLVECNNHYLCLNCLTLLLSVSNRCPICKMPLPTKLRPS
AAPTAPPTGAADSIRPPPYSP

Therefore, preferably the RNA construct comprises a nucleotide sequence which encodes an amino acid sequence substantially as set out in SEQ ID No: 131, or a variant or fragment thereof.

In one embodiment, the LASV Z polypeptide is encoded by the DNA nucleotide sequence of SEQ ID No: 132, as follows:

[SEQ ID No: 132]
ATGGGAAACAAGCAAGCCAAAGCCCCAGAATCAAAAGACAGTCCGAGAGCCAGCCTGATCCCAGATGCCACACATCTA
GGGCCACAGTTCTGTAAGAGCTGCTGGTTCGAAAACAAGGGCCTGGTTGAGTGCAACAACCACTATCTGTGTCTCAAC
TGCCTCACCTTACTTCTAAGTGTCAGCAACAGGTGTCCCATTTGCAAGATGCCTCTCCCCACAAAACTGAGACCATCA
GCCGCTCCAACAGCACCTCCAACCGGAGCAGCGGACAGCATCAGACCCCCACCCTACAGTCCC

Accordingly, preferably the LASV Z polypeptide is encoded by the DNA nucleotide sequence substantially as set out in SEQ ID NO: 132, or a variant or fragment thereof.

One embodiment of codon optimised nucleic acid (DNA) encoding the LASV Z polypeptide is provided herein as SEQ ID No: 133, as follows:

[SEQ ID No: 133]
ATGGGCAACAAGCAGGCCAAGGCTCCCGAGAGCAAGGATAGCCCTAGAGCCTCTCTGATCCCCGACGCCACACATCTG
GGACCCCAGTTCTGCAAGAGCTGTTGGTTCGAGAACAAAGGCCTGGTGGAATGCAACAACCACTACCTGTGCCTGAAC
TGTCTGACCCTGCTGCTGAGCGTGTCCAACAGATGCCCCATCTGCAAGATGCCCCTGCCTACCAAGCTGAGGCCTTCT
GCTGCTCCTACAGCTCCTCCAACAGGCGCCGCTGATAGCATCAGACCTCCACCTTATAGCCCC

Hence, preferably the RNA construct is encoded by a DNA sequence substantially as set out in SEQ ID No: 133, or a fragment or variant thereof.

In an embodiment, the RNA sequence corresponding to the codon optimised DNA sequence of SEQ ID No: 133 is provided herein as SEQ ID No: 134, as follows:

[SEQ ID No: 134]
AUGGGCAACAAGCAGGCCAAGGCUCCCGAGAGCAAGGAUAGCCCUAGAGCCUCUCUGAUCCCCGACGCCACACAUCUG
GGACCCCAGUUCUGCAAGAGCUGUUGGUUCGAGAACAAAGGCCUGGUGGAAUGCAACAACCACUACCUGUGCCUGAAC
UGUCUGACCCUGCUGCUGAGCGUGUCCAACAGAUGCCCCAUCUGCAAGAUGCCCCUGCCUACCAAGCUGAGGCCUUCU
GCUGCUCCUACAGCUCCUCCAACAGGCGCCGCUGAUAGCAUCAGACCUCCACCUUAUAGCCCC

Hence, preferably the RNA construct comprises a sequence substantially as set out in SEQ ID No: 134, or a fragment or variant thereof.

In one embodiment, the at least one IIP is DANV Z protein (B1NX60; RING finger protein Z Dandenong virus), or an orthologue thereof. One embodiment of the polypeptide sequence of DANV Z is represented herein as SEQ ID No: 135, as follows:

[SEQ ID No: 135]
MGQAKSKETKLSKKEDRAEVLPDATYLGPLNCKSCWQRFDSLVRCHDHYLCRQCLNLLLTVSDRCPLCKHPLPTKLRV
STAPSSPPPYEE

Therefore, preferably the RNA construct comprises a nucleotide sequence which encodes an amino acid sequence substantially as set out in SEQ ID No: 135, or a variant or fragment thereof.

In one embodiment, the DANV Z polypeptide is encoded by the DNA nucleotide sequence of SEQ ID No: 136, as follows:

[SEQ ID No: 136]
ATGGGTCAAGCAAAATCCAAAGAAACAAAGCTCTCCAAGAAAGAGGACAGAGCAGAGGTTCTACCTGACGCAACCTAT
CTTGGTCCTCTGAACTGCAAATCATGCTGGCAAAGGTTCGACAGTTTGGTTAGGTGCCATGACCACTACCTATGCAGG
CAATGTCTGAACCTTTTGTTGACAGTCTCAGACAGATGCCCTCTCTGCAAACACCCTCTACCGACCAAGCTGAGGGTG
TCGACAGCCCCCAGCTCACCTCCCCCCTACGAGGAG

Accordingly, preferably the DANV Z polypeptide is encoded by the DNA nucleotide sequence substantially as set out in SEQ ID NO: 136, or a variant or fragment thereof.

One embodiment of codon optimised nucleic acid (DNA) encoding the DANV Z polypeptide is provided herein as SEQ ID No: 137, as follows:

[SEQ ID No: 137]
ATGGGCCAAGCCAAGAGCAAAGAGACAAAGCTGAGCAAGAAAGAGGACCGCGCCGAGGTTCTGCCCGATGCCACATAT
CTGGGCCCTCTGAACTGCAAGAGCTGCTGGCAGAGATTCGACAGCCTCGTGCGGTGCCACGATCACTACCTGTGCAGA
CAGTGCCTGAACCTGCTGCTGACCGTGTCCGATAGATGCCCTCTGTGCAAGCACCCTCTGCCTACCAAGCTGAGAGTG
TCCACCGCTCCTAGCAGCCCTCCACCTTATGAGGAA

Hence, preferably the RNA construct is encoded by a DNA sequence substantially as set out in SEQ ID No: 137, or a fragment or variant thereof.

In an embodiment, the RNA sequence corresponding to the codon optimised DNA sequence of SEQ ID No: 137 is provided herein as SEQ ID No: 138, as follows:

[SEQ ID No: 138]
AUGGGCCAAGCCAAGAGCAAAGAGACAAAGCUGAGCAAGAAAGAGGACCGCGCCGAGGUUCUGCCCGAUGCCACAUAU
CUGGGCCCUCUGAACUGCAAGAGCUGCUGGCAGAGAUUCGACAGCCUCGUGCGGUGCCACGAUCACUACCUGUGCAGA
CAGUGCCUGAACCUGCUGCUGACCGUGUCCGAUAGAUGCCCUCUGUGCAAGCACCCUCUGCCUACCAAGCUGAGAGUG
UCCACCGCUCCUAGCAGCCCUCCACCUUAUGAGGAA

Hence, preferably the RNA construct comprises a sequence substantially as set out in SEQ ID No: 138, or a fragment or variant thereof.

In one embodiment, the at least one IIP is LCMV Z protein (P18541; RING finger protein Z Lymphocytic choriomeningitis virus (strain Armstong), or an orthologue thereof. One embodiment of the polypeptide sequence of LCMV Z protein is represented herein as SEQ ID No: 139, as follows:

[SEQ ID No: 139]
MGQGKSREEKGINSTNRAEILPDTTYLGPLSCKSCWQKFDSLVRCHDHYLCRHCLNLLLSVSDRCPLCKYPLPTRLKI
STAPSSPPPYEE

Therefore, preferably the RNA construct comprises a nucleotide sequence which encodes an amino acid sequence substantially as set out in SEQ ID No: 139, or a variant or fragment thereof.

In one embodiment, the LCMV Z polypeptide is encoded by the DNA nucleotide sequence of SEQ ID No: 140, as follows:

[SEQ ID No: 140]
ATGGGTCAAGGCAAGTCCAGAGAGGAGAAAGGCACCAATAGTACAAACAGGGCCGAAATCCTACCAGATACCACCTAT
CTTGGCCCTTTAAGCTGCAAATCTTGCTGGCAGAAATTTGACAGCTTGGTAAGATGCCATGACCACTACCTTTGCAGG
CACTGTTTAAACCTTCTGCTGTCAGTATCCGACAGGTGTCCTCTTTGTAAATATCCATTACCAACCAGATTGAAGATA
TCAACAGCCCCAAGCTCTCCACCTCCCTACGAAGAG

Accordingly, preferably the LCMV Z polypeptide is encoded by the DNA nucleotide sequence substantially as set out in SEQ ID NO: 140, or a variant or fragment thereof.

One embodiment of codon optimised nucleic acid (DNA) encoding the LCMV Z polypeptide is provided herein as SEQ ID No: 141, as follows:

[SEQ ID No: 141]
ATGGGCCAGGGCAAGTCCAGAGAGGAAAAGGGCACCAACTCCACCAACCGGGCCGAGATCCTGCCTGACACCACATAT
CTGGGCCCTCTGAGCTGCAAGAGCTGCTGGCAGAAATTCGACAGCCTCGTGCGGTGCCACGACCACTACCTGTGTAGA
CACTGCCTGAACCTGCTGCTGAGCGTGTCCGATAGATGCCCTCTGTGCAAGTACCCTCTGCCTACCAGACTGAAGATC
AGCACAGCCCCTAGCAGCCCTCCACCTTACGAAGAA

Hence, preferably the RNA construct is encoded by a DNA sequence substantially as set out in SEQ ID No: 141, or a fragment or variant thereof.

In an embodiment, the RNA sequence corresponding to the codon optimised DNA sequence of SEQ ID No: 141 is provided herein as SEQ ID No: 142, as follows:

[SEQ ID No: 142]
AUGGGCCAGGGCAAGUCCAGAGAGGAAAAGGGCACCAACUCCACCAACCGGGCCGAGAUCCUGCCUGACACCACAUAU
CUGGGCCCUCUGAGCUGCAAGAGCUGCUGGCAGAAAUUCGACAGCCUCGUGCGGUGCCACGACCACUACCUGUGUAGA
CACUGCCUGAACCUGCUGCUGAGCGUGUCCGAUAGAUGCCCUCUGUGCAAGUACCCUCUGCCUACCAGACUGAAGAUC
AGCACAGCCCCUAGCAGCCCUCCACCUUACGAAGAA

Hence, preferably the RNA construct comprises a sequence substantially as set out in SEQ ID No: 142, or a fragment or variant thereof.

In one embodiment, the at least one IIP is LUJV Z protein (C5ILC3; Multifunctional matrix-like protein Z Lujp mammarenavirus), or an orthologue thereof. One embodiment of the polypeptide sequence of LUJV Z protein is represented herein as SEQ ID No: 143, as follows:

[SEQ ID No: 143]
MGQRHSSGSGQPNPKPSDSDHEARRSELHSDASHLGPLNCKSCWKSKKALVKCYDHYLCLNCLSLLMGITPRCPFCYR
ELPKNLDLAEAPSAPPL

Therefore, preferably the RNA construct comprises a nucleotide sequence which encodes an amino acid sequence substantially as set out in SEQ ID No: 143, or a variant or fragment thereof.

In one embodiment, the LUJV Z polypeptide is encoded by the DNA nucleotide sequence of SEQ ID No: 144, as follows:

[SEQ ID No: 144]
ATGGGTCAGAGACATTCCTCTGGCTCCGGCCAACCCAACCCCAAGCCGAGTGACAGCGATCATGAAGCGAGAAGGTCA
GAGCTTCACTCGGACGCCTCCCATCTCGGACCTCTGAACTGCAAATCTTGCTGGAAGTCAAAGAAGGCACTGGTGAAG
TGCTATGATCACTACCTCTGTCTAAACTGCTTAAGCCTTCTAATGGGCATCACCCCAAGATGTCCCTTCTGTTACAGA
GAGCTACCCAAAAATCTGGACCTTGCAGAGGCACCAAGCGCACCACCCCTC

Accordingly, preferably the LUJV Z polypeptide is encoded by the DNA nucleotide sequence substantially as set out in SEQ ID NO: 144, or a variant or fragment thereof.

One embodiment of codon optimised nucleic acid (DNA) encoding the LUJV Z polypeptide is provided herein as SEQ ID No: 145, as follows:

[SEQ ID No: 145]
ATGGGCCAGAGACACAGCTCTGGAAGCGGCCAGCCTAATCCTAAGCCTAGCGACAGCGATCACGAGGCCAGAAGAAGC
GAGCTGCACTCCGATGCCTCTCACCTGGGACCTCTGAACTGCAAGAGCTGCTGGAAGTCCAAGAAAGCCCTGGTCAAG
TGCTACGACCACTACCTGTGCCTGAACTGCCTGAGCCTGCTGATGGGCATTACCCCTAGATGCCCCTTCTGCTACAGA
GAGCTGCCCAAGAACCTGGACCTGGCCGAAGCTCCTTCTGCTCCTCCTCTG

Hence, preferably the RNA construct is encoded by a DNA sequence substantially as set out in SEQ ID No: 145, or a fragment or variant thereof.

In an embodiment, the RNA sequence corresponding to the codon optimised DNA sequence of SEQ ID No: 145 is provided herein as SEQ ID No: 146, as follows:

[SEQ ID No: 146]
AUGGGCCAGAGACACAGCUCUGGAAGCGGCCAGCCUAAUCCUAAGCCUAGCGACAGCGAUCACGAGGCCAGAAGAAGC
GAGCUGCACUCCGAUGCCUCUCACCUGGGACCUCUGAACUGCAAGAGCUGCUGGAAGUCCAAGAAAGCCCUGGUCAAG
UGCUACGACCACUACCUGUGCCUGAACUGCCUGAGCCUGCUGAUGGGCAUUACCCCUAGAUGCCCCUUCUGCUACAGA
GAGCUGCCCAAGAACCUGGACCUGGCCGAAGCUCCUUCUGCUCCUCCUCUG

Hence, preferably the RNA construct comprises a sequence substantially as set out in SEQ ID No: 146, or a fragment or variant thereof.

In one embodiment, the at least one IIP is CHPV Z protein (B2C4J2; RING finger protein Z Chapare mammarenavirus (isolate Human/Bolivia/810419/2003), or an orthologue thereof. One embodiment of the polypeptide sequence of CHPV Z protein is represented herein as SEQ ID No: 147, as follows:

[SEQ ID No: 147]
MGNTKTKDRQYQSNSSQPTNTSAPVLLRRQAEPSLYGRHNCRCCWFADTNLVNCSNHYLCLKCLNTMLRRSNLCDICG
EELPTTIIVPVEPSAPLPGQ

Therefore, preferably the RNA construct comprises a nucleotide sequence which encodes an amino acid sequence substantially as set out in SEQ ID No: 147, or a variant or fragment thereof.

In one embodiment, the CHPV Z polypeptide is encoded by the DNA nucleotide sequence of SEQ ID No: 148, as follows:

[SEQ ID No: 148]
ATGGGTAACACCAAAACAAAGGACAGACAGTATCAATCGAACTCAAGCCAACCCACAAACACATCTGCACCAGTTCTG
CTGAGAAGGCAGGCAGAACCAAGTCTGTATGGGAGACACAACTGCAGATGCTGTTGGTTTGCAGACACAAACCTAGTC
AATTGTTCCAACCACTACCTTTGCCTTAAATGTCTGAACACAATGTTAAGAAGATCCAATCTCTGTGACATATGCGGT
GAAGAACTTCCCACAACAATCATTGTTCCAGTGGAACCATCAGCACCACTGCCCGGACAA

Accordingly, preferably the CHPV Z polypeptide is encoded by the DNA nucleotide sequence substantially as set out in SEQ ID NO: 148, or a variant or fragment thereof.

One embodiment of codon optimised nucleic acid (DNA) encoding the CHPV Z polypeptide is provided herein as SEQ ID No: 149, as follows:

[SEQ ID No: 149]
ATGGGCAACACCAAGACCAAGGACCGGCAGTACCAGAGCAACAGCAGCCAGCCTACCAACACATCTGCCCCTGTGCTG
CTGAGAAGGCAGGCCGAGCCTTCTCTGTACGGCAGACACAACTGCCGGTGCTGTTGGTTCGCCGACACCAACCTGGTC
AACTGCAGCAACCACTACCTGTGCCTGAAGTGTCTGAACACCATGCTGCGGCGGAGCAACCTGTGCGATATCTGTGGC
GAGGAACTGCCCACCACCATCATCGTGCCTGTGGAACCTTCTGCTCCTCTGCCTGGACAG

Hence, preferably the RNA construct is encoded by a DNA sequence substantially as set out in SEQ ID No: 149, or a fragment or variant thereof.

In an embodiment, the RNA sequence corresponding to the codon optimised DNA sequence of SEQ ID No: 149 is provided herein as SEQ ID No: 150, as follows:

[SEQ ID No: 150]
AUGGGCAACACCAAGACCAAGGACCGGCAGUACCAGAGCAACAGCAGCCAGCCUACCAACACAUCUGCCCCUGUGCUG
CUGAGAAGGCAGGCCGAGCCUUCUCUGUACGGCAGACACAACUGCCGGUGCUGUUGGUUCGCCGACACCAACCUGGUC
AACUGCAGCAACCACUACCUGUGCCUGAAGUGUCUGAACACCAUGCUGCGGCGGAGCAACCUGUGCGAUAUCUGUGGC
GAGGAACUGCCCACCACCAUCAUCGUGCCUGUGGAACCUUCUGCUCCUCUGCCUGGACAG

Hence, preferably the RNA construct comprises a sequence substantially as set out in SEQ ID No: 150, or a fragment or variant thereof.

In one embodiment, the at least one IIP is MACV Z protein (Q6IUF9; RING finger protein Z Machupo virus), or an orthologue thereof. One embodiment of the polypeptide sequence of MACV Z protein is represented herein as SEQ ID No: 151, as follows:

[SEQ ID No: 151]
MGNCNKPPKRPPNTQTSAAQPSAEFRRTALPSLYGRYNCKCCWFADTNLITCNDHYLCLRCHQTMLRNSELCHICWKP
LPTSITVPVEPSAPPP

Therefore, preferably the RNA construct comprises a nucleotide sequence which encodes an amino acid sequence substantially as set out in SEQ ID No: 151, or a variant or fragment thereof.

In one embodiment, the MACV Z polypeptide is encoded by the DNA nucleotide sequence of SEQ ID No: 152, as follows:

[SEQ ID No: 152]
ATGGGCAACTGTAACAAGCCTCCCAAGAGGCCTCCCAACACACAAACATCAGCCGCCCAGCCCTCAGCAGAGTTCAGG
AGAACAGCCCTACCCAGTCTCTATGGTCGCTACAACTGCAAATGTTGTTGGTTTGCCGACACAAACTTGATTACATGT
AACGACCACTACTTGTGTCTGAGATGTCATCAAACAATGCTCAGGAATTCTGAACTCTGTCACATATGCTGGAAACCA
CTACCGACATCCATCACAGTCCCCGTGGAGCCAAGCGCCCCCCCACCA

Accordingly, preferably the MACV Z polypeptide is encoded by the DNA nucleotide sequence substantially as set out in SEQ ID NO: 152, or a variant or fragment thereof.

One embodiment of codon optimised nucleic acid (DNA) encoding the MACV Z polypeptide is provided herein as SEQ ID No: 153, as follows:

[SEQ ID No: 153]
ATGGGCAACTGCAACAAGCCTCCTAAGCGGCCTCCTAACACACAGACATCTGCCGCTCAGCCTAGCGCCGAGTTCAGA
AGAACAGCCCTGCCTAGCCTGTACGGCCGGTACAACTGCAAGTGCTGTTGGTTCGCCGACACCAACCTGATCACCTGT
AACGACCACTACCTGTGCCTGCGGTGCCACCAGACCATGCTGAGAAATAGCGAGCTGTGCCACATCTGCTGGAAGCCC
CTGCCTACCAGCATCACCGTGCCTGTGGAACCTTCTGCTCCTCCTCCT

Hence, preferably the RNA construct is encoded by a DNA sequence substantially as set out in SEQ ID No: 153, or a fragment or variant thereof.

In an embodiment, the RNA sequence corresponding to the codon optimised DNA sequence of SEQ ID No: 153 is provided herein as SEQ ID No: 154, as follows:

[SEQ ID No: 154]
AUGGGCAACUGCAACAAGCCUCCUAAGCGGCCUCCUAACACACAGACAUCUGCCGCUCAGCCUAGCGCCGAGUUCAGA
AGAACAGCCCUGCCUAGCCUGUACGGCCGGUACAACUGCAAGUGCUGUUGGUUCGCCGACACCAACCUGAUCACCUGU
AACGACCACUACCUGUGCCUGCGGUGCCACCAGACCAUGCUGAGAAAUAGCGAGCUGUGCCACAUCUGCUGGAAGCCC
CUGCCUACCAGCAUCACCGUGCCUGUGGAACCUUCUGCUCCUCCUCCU

Hence, preferably the RNA construct comprises a sequence substantially as set out in SEQ ID No: 154, or a fragment or variant thereof.

In one embodiment, the at least one IIP is GTOV Z protein (Q6UY71; RING finger protein Z Guanarito mammarenavirus (isolate Human/Venezuela/NH-95551/1990), or an orthologue thereof. One embodiment of the polypeptide sequence of GTOV Z protein is represented herein as SEQ ID No: 155, as follows:

[SEQ ID No: 155]
MGNSKSKSNPSSSSESQKGAPTVTEFRRTAIHSLYGRYNCKCCWFADKNLIKCSDHYLCLRCLNVMLKNSDLCNICWE
QLPTCITVPEEPSAPPE

Therefore, preferably the RNA construct comprises a nucleotide sequence which encodes an amino acid sequence substantially as set out in SEQ ID No: 155, or a variant or fragment thereof.

In one embodiment, the GTOV Z polypeptide is encoded by the DNA nucleotide sequence of SEQ ID No: 156, as follows:

[SEQ ID No: 156]
ATGGGCAATTCAAAATCTAAATCCAACCCATCCAGTTCCTCAGAGTCTCAAAAAGGGGCACCAACAGTCACAGAATTT
AGGAGGACTGCCATTCACAGTCTCTATGGGAGGTACAACTGCAAGTGTTGCTGGTTTGCTGACAAGAATCTGATTAAA
TGCTCTGATCATTACCTCTGCTTGAGGTGTTTAAATGTCATGCTGAAAAACTCTGATCTTTGCAACATTTGTTGGGAG
CAGCTGCCCACATGCATCACAGTTCCGGAGGAGCCAAGCGCTCCACCGGAA

Accordingly, preferably the GTOV Z polypeptide is encoded by the DNA nucleotide sequence substantially as set out in SEQ ID NO: 156, or a variant or fragment thereof.

One embodiment of codon optimised nucleic acid (DNA) encoding the GTOV Z polypeptide is provided herein as SEQ ID No: 157, as follows:

[SEQ ID No: 157]
ATGGGCAACAGCAAGTCCAAGAGCAACCCCAGCAGCAGCTCCGAGTCTCAGAAAGGCGCTCCTACCGTGACCGAGTTC
AGAAGAACAGCCATCCACAGCCTGTACGGCCGGTACAACTGCAAGTGCTGTTGGTTCGCCGACAAGAACCTGATCAAG
TGCAGCGACCACTACCTGTGCCTGCGGTGCCTGAACGTGATGCTGAAGAACTCCGACCTGTGCAACATCTGCTGGGAG
CAGCTGCCTACCTGCATCACCGTGCCTGAGGAACCTTCTGCTCCTCCTGAA

Hence, preferably the RNA construct is encoded by a DNA sequence substantially as set out in SEQ ID No: 157, or a fragment or variant thereof.

In an embodiment, the RNA sequence corresponding to the codon optimised DNA sequence of SEQ ID No: 157 is provided herein as SEQ ID No: 158, as follows:

[SEQ ID No: 158]
AUGGGCAACAGCAAGUCCAAGAGCAACCCCAGCAGCAGCUCCGAGUCUCAGAAAGGCGCUCCUACCGUGACCGAGUUC
AGAAGAACAGCCAUCCACAGCCUGUACGGCCGGUACAACUGCAAGUGCUGUUGGUUCGCCGACAAGAACCUGAUCAAG
UGCAGCGACCACUACCUGUGCCUGCGGUGCCUGAACGUGAUGCUGAAGAACUCCGACCUGUGCAACAUCUGCUGGGAG
CAGCUGCCUACCUGCAUCACCGUGCCUGAGGAACCUUCUGCUCCUCCUGAA

Hence, preferably the RNA construct comprises a sequence substantially as set out in SEQ ID No: 158, or a fragment or variant thereof.

In one embodiment, the at least one IIP is JUNV Z protein (Q6IVU5; RING finger protein Z OS=Junin mammarenavirus), or an orthologue thereof. One embodiment of the polypeptide sequence of JUNV Z is represented herein as SEQ ID No: 159, as follows:

[SEQ ID No: 159]
MGNCNGASKSNQPDSSRVTQPAAEFRRVAHSSLYGRYNCKCCWFADTNLITCNDHYLCLRCHQVMLRNSDLCNICWKP
LPTTITVPVEPTAPPP

Therefore, preferably the RNA construct comprises a nucleotide sequence which encodes an amino acid sequence substantially as set out in SEQ ID No: 159, or a variant or fragment thereof.

In one embodiment, the JUNV Z polypeptide is encoded by the DNA nucleotide sequence of SEQ ID No: 160, as follows:

[SEQ ID No: 160]
ATGGGCAACTGCAACGGGGCATCCAAGTCAAACCAGCCAGACTCTTCAAGGGTCACACAGCCAGCCGCAGAATTCAGG
AGGGTGGCTCACAGCAGTCTATATGGTAGATACAACTGCAAGTGCTGCTGGTTTGCTGACACCAATCTGATAACCTGC
AATGATCACTACCTTTGTTTAAGGTGCCATCAGGTTATGTTAAGGAATTCGGACCTCTGCAATATCTGTTGGAAGCCC
CTACCTACCACAATCACAGTGCCGGTGGAGCCAACAGCACCACCACCA

Accordingly, preferably the JUNV Z polypeptide is encoded by the DNA nucleotide sequence substantially as set out in SEQ ID NO: 160, or a variant or fragment thereof.

One embodiment of codon optimised nucleic acid (DNA) encoding the JUNV Z polypeptide is provided herein as SEQ ID No: 161, as follows:

[SEQ ID No: 161]
ATGGGCAATTGCAACGGCGCCAGCAAGAGCAACCAGCCTGATAGCAGCAGAGTGACACAGCCTGCCGCCGAGTTTAGA
AGAGTGGCCCACAGCAGCCTGTACGGCCGGTACAATTGCAAGTGCTGTTGGTTCGCCGACACCAACCTGATCACCTGT
AACGACCACTACCTGTGCCTGCGGTGCCACCAAGTGATGCTGAGAAACAGCGACCTGTGCAACATCTGCTGGAAGCCC
CTGCCTACCACCATCACCGTGCCTGTGGAACCTACAGCTCCTCCTCCT

Hence, preferably the RNA construct is encoded by a DNA sequence substantially as set out in SEQ ID No: 161, or a fragment or variant thereof.

In an embodiment, the RNA sequence corresponding to the codon optimised DNA sequence of SEQ ID No: 161 is provided herein as SEQ ID No: 162, as follows:

[SEQ ID No: 162]
AUGGGCAAUUGCAACGGCGCCAGCAAGAGCAACCAGCCUGAUAGCAGCAGAGUGACACAGCCUGCCGCCGAGUUUAGA
AGAGUGGCCCACAGCAGCCUGUACGGCCGGUACAAUUGCAAGUGCUGUUGGUUCGCCGACACCAACCUGAUCACCUGU
AACGACCACUACCUGUGCCUGCGGUGCCACCAAGUGAUGCUGAGAAACAGCGACCUGUGCAACAUCUGCUGGAAGCCC
CUGCCUACCACCAUCACCGUGCCUGUGGAACCUACAGCUCCUCCUCCU

Hence, preferably the RNA construct comprises a sequence substantially as set out in SEQ ID No: 162, or a fragment or variant thereof.

In one embodiment, the at least one IIP is SABV Z protein (Q6UY62; RING finger protein Z Sabia mammarenavirus (isolate Human/Brasil/SPH114202/1990)), or an orthologue thereof. One embodiment of the polypeptide sequence of SABV Z protein is represented herein as SEQ ID No: 163, as follows:

[SEQ ID No: 163]
MGNSKSKSKLSANQYEQQTVNSTKQVAILKRQAEPSLYGRHNCRCCWFANTNLIKCSDHYICLKCLNIMLGKSSFCDI
CGEELPTSIVVPIEPSAPPPED

Therefore, preferably the RNA construct comprises a nucleotide sequence which encodes an amino acid sequence substantially as set out in SEQ ID No: 163, or a variant or fragment thereof.

In one embodiment, the SABV Z polypeptide is encoded by the DNA nucleotide sequence of SEQ ID No: 164, as follows:

[SEQ ID No: 164]
ATGGGTAACTCCAAGTCAAAATCAAAGCTGTCTGCTAACCAGTATGAACAGCAAACAGTCAATAGCACCAAACAGGTA
GCCATTTTAAAGAGACAGGCTGAACCTAGTCTGTATGGAAGACACAACTGCAGGTGCTGCTGGTTCGCCAATACAAAT
CTAATAAAATGTTCCGACCATTATATTTGTCTAAAATGTTTGAACATAATGTTGGGGAAGTCTTCTTTTTGTGACATT
TGTGGTGAAGAGCTCCCCACATCCATTGTGGTGCCCATCGAACCAAGTGCTCCACCTCCAGAAGAC

Accordingly, preferably the SABV Z polypeptide is encoded by the DNA nucleotide sequence substantially as set out in SEQ ID NO: 164, or a variant or fragment thereof.

One embodiment of codon optimised nucleic acid (DNA) encoding the SABV Z polypeptide is provided herein as SEQ ID No: 165, as follows:

[SEQ ID No: 165]
ATGGGCAACAGCAAGAGCAAGTCCAAGCTGAGCGCCAACCAGTACGAGCAGCAGACCGTGAACAGCACCAAACAGGTG
GCCATCCTGAAGAGACAGGCCGAGCCTAGCCTGTACGGCAGACACAACTGCCGGTGTTGTTGGTTCGCCAACACCAAC
CTGATCAAGTGCAGCGACCACTACATCTGCCTGAAGTGCCTGAACATCATGCTGGGCAAGAGCAGCTTCTGCGACATC
TGCGGAGAGGAACTGCCCACCTCTATCGTGGTGCCTATCGAGCCTTCTGCTCCTCCACCTGAGGAT

Hence, preferably the RNA construct is encoded by a DNA sequence substantially as set out in SEQ ID No: 165, or a fragment or variant thereof.

In an embodiment, the RNA sequence corresponding to the codon optimised DNA sequence of SEQ ID No: 165 is provided herein as SEQ ID No: 166, as follows:

[SEQ ID No: 166]
AUGGGCAACAGCAAGAGCAAGUCCAAGCUGAGCGCCAACCAGUACGAGCAGCAGACCGUGAACAGCACCAAACAGGUG
GCCAUCCUGAAGAGACAGGCCGAGCCUAGCCUGUACGGCAGACACAACUGCCGGUGUUGUUGGUUCGCCAACACCAAC
CUGAUCAAGUGCAGCGACCACUACAUCUGCCUGAAGUGCCUGAACAUCAUGCUGGGCAAGAGCAGCUUCUGCGACAUC
UGCGGAGAGGAACUGCCCACCUCUAUCGUGGUGCCUAUCGAGCCUUCUGCUCCUCCACCUGAGGAU

Hence, preferably the RNA construct comprises a sequence substantially as set out in SEQ ID No: 166, or a fragment or variant thereof.

In other embodiments, the at least one IIP may be a 3C protease protein.

In one embodiment, the at least one IIP is CV-A16 3C protease (Q9QF31; Coxsackievirus A16 (strain Tainan/5079/98 Protease 3C), or an orthologue thereof (Rui Y, Su Jm Wang H, Chang J, Wang S, Zhenf W, Cai Y, Gordy J T, Markham R, Kong W, Zhang W, Yu X-F. (2017) Disruption of MDA5-Mediated Innate Immune Responses by the 3C Proteins of Coxsackievirus A16, Coxsackievirus A6, and Enterovirus D68. J Virol 91, 13, e00546-17). One embodiment of the polypeptide sequence of CV-A16 3C protease is represented herein as SEQ ID No: 167, as follows:

[SEQ ID No: 167]
GPSLDFALSLLRRNIRQVQTDQGHFTMLGVRDRLAILPRHSQPGKTIWVEHKLINVLDAVELVDEQGVNLELTLVTLD
TNEKFRDVTKFIPETITGASDATLVINTEHMPSMFVPVGDVVQYGFLNLSGKPTHRTMMYNFPTKAGQCGGVVTSVGK
IIGIHIGGNGRQGFCAGLKRGYFASEQ

Therefore, preferably the RNA construct comprises a nucleotide sequence which encodes an amino acid sequence substantially as set out in SEQ ID No: 167, or a variant or fragment thereof.

In one embodiment, the CV-A16 3C protease polypeptide is encoded by the DNA nucleotide sequence of SEQ ID No: 168, as follows:

[SEQ ID No: 168]
GGACCGAGCTTAGACTTTGCCTTATCCCTCCTAAGGCGCAACATTAGACAGGTGCAAACCGACCAAGGACACTTCACT
ATGTTAGGGGTGCGAGATCGCCTAGCCATTTTGCCACGCCACTCGCAACCAGGAAAAACTATCTGGGTGGAGCACAAG
TTAATTAATGTGCTGGATGCTGTCGAATTAGTGGATGAGCAAGGTGTAAACTTGGAACTCACACTAGTAACCTTAGAC
ACCAACGAAAAGTTTAGGGATGTTACCAAGTTTATTCCAGAGACGATCACCGGGGCAAGCGACGCAACCTTGGTCATC
AACACTGAGCACATGCCCTCAATGTTCGTTCCAGTGGGTGATGTTGTACAATATGGATTTCTGAATCTCAGCGGTAAG
CCCACACACCGAACCATGATGTACAATTTCCCCACAAAGGCAGGACAGTGTGGAGGGGTGGTCACCTCAGTCGGTAAG
ATCATAGGAATTCACATTGGTGGGAATGGACGCCAGGGTTTCTGCGCTGGACTGAAGAGAGGCTATTTTGCCAGTGAA
CAG

Accordingly, preferably the CV-A16 3C protease polypeptide is encoded by the DNA nucleotide sequence substantially as set out in SEQ ID NO: 168, or a variant or fragment thereof.

One embodiment of codon optimised nucleic acid (DNA) encoding the CV-A16 3C protease polypeptide is provided herein as SEQ ID No: 169, as follows:

[SEQ ID No: 169]
GGCCCTTCTCTGGATTTTGCCCTGAGCCTGCTGCGGCGGAACATCAGACAGGTGCAGACAGATCAGGGCCACTTCACC
ATGCTGGGCGTCAGAGACAGACTGGCCATCCTGCCTAGACACAGCCAGCCTGGCAAGACCATCTGGGTCGAGCACAAG
CTGATCAACGTGCTGGACGCCGTGGAACTGGTGGATGAACAGGGCGTGAACCTGGAACTGACCCTGGTCACCCTGGAC
ACCAACGAGAAGTTCCGGGACGTGACCAAGTTCATCCCCGAGACAATCACCGGCGCCTCCGATGCCACACTGGTCATC
AATACCGAGCACATGCCCTCCATGTTCGTGCCTGTGGGAGATGTGGTGCAGTACGGCTTCCTGAACCTGAGCGGCAAG
CCCACACACCGGACCATGATGTACAACTTCCCTACCAAGGCCGGCCAGTGCGGCGGAGTGGTTACATCTGTGGGCAAG
ATCATCGGCATCCACATCGGCGGCAATGGCAGACAGGGATTTTGTGCCGGCCTGAAGAGAGGCTACTTCGCCTCTGAA
CAG

Hence, preferably the RNA construct is encoded by a DNA sequence substantially as set out in SEQ ID No: 169, or a fragment or variant thereof.

In an embodiment, the RNA sequence corresponding to the codon optimised DNA sequence of SEQ ID No: 169 is provided herein as SEQ ID No: 170, as follows:

[SEQ ID No: 170]
GGCCCUUCUCUGGAUUUUGCCCUGAGCCUGCUGCGGCGGAACAUCAGACAGGUGCAGACAGAUCAGGGCCACUUCACC
AUGCUGGGCGUCAGAGACAGACUGGCCAUCCUGCCUAGACACAGCCAGCCUGGCAAGACCAUCUGGGUCGAGCACAAG
CUGAUCAACGUGCUGGACGCCGUGGAACUGGUGGAUGAACAGGGCGUGAACCUGGAACUGACCCUGGUCACCCUGGAC
ACCAACGAGAAGUUCCGGGACGUGACCAAGUUCAUCCCCGAGACAAUCACCGGCGCCUCCGAUGCCACACUGGUCAUC
AAUACCGAGCACAUGCCCUCCAUGUUCGUGCCUGUGGGAGAUGUGGUGCAGUACGGCUUCCUGAACCUGAGCGGCAAG
CCCACACACCGGACCAUGAUGUACAACUUCCCUACCAAGGCCGGCCAGUGCGGCGGAGUGGUUACAUCUGUGGGCAAG
AUCAUCGGCAUCCACAUCGGCGGCAAUGGCAGACAGGGAUUUUGUGCCGGCCUGAAGAGAGGCUACUUCGCCUCUGAA
CAG

Hence, preferably the RNA construct comprises a sequence substantially as set out in SEQ ID No: 170, or a fragment or variant thereof.

In one embodiment, the at least one IIP is CV-A6 3C protease (L7P6C4; Genome polyprotein Coxsackievirus A6 Protease 3C), or an orthologue thereof. One embodiment of the polypeptide sequence of CV-A6 3C protease is represented herein as SEQ ID No: 171, as follows:

[SEQ ID No: 171]
GPSLDFALSLLRRNIRQVQTDQGHFTMLGVRDRLAVLPRHSQPGKTIWVEHKLVNVVDAVELVDEQGVNLELTLITLD
TNEKFRDITKFIPENISAASDATLVINTEHMPSMFVPVGDVVQYGFLNLSGKPTHRTMMYNFPTKAGQCGGVVTSVGK
VIGIHIGGNGRQGFCAGLKRSYF

Therefore, preferably the RNA construct comprises a nucleotide sequence which encodes an amino acid sequence substantially as set out in SEQ ID No: 171, or a variant or fragment thereof.

In one embodiment, the CV-A6 3C protease polypeptide is encoded by the DNA nucleotide sequence of SEQ ID No: 172, as follows:

[SEQ ID No: 172]
GGACCTAGCCTTGATTTTGCCCTATCCCTACTGAGAAGGAACATCAGACAAGTTCAAACGGACCAAGGGCACTTCACC
ATGCTAGGAGTCAGAGATCGCTTGGCCGTCCTCCCACGACACTCACAACCCGGAAAAACTATCTGGGTAGAGCACAAG
CTAGTGAATGTTGTGGATGCTGTCGAACTAGTGGATGAGCAGGGGGTCAACTTAGAGCTGACTTTAATCACCCTCGAC
ACTAATGAGAAGTTTAGAGACATCACCAAATTTATTCCAGAGAATATCAGCGCTGCCAGCGATGCCACTCTAGTGATT
AATACAGAGCACATGCCTTCTATGTTTGTGCCAGTAGGTGATGTTGTTCAGTATGGTTTCCTGAATCTTAGTGGGAAA
CCAACCCACCGCACTATGATGTATAACTTCCCTACCAAGGCAGGACAGTGTGGAGGGGTAGTCACATCAGTTGGAAAG
GTCATTGGTATCCACATAGGAGGCAATGGCAGGCAAGGTTTCTGTGCAGGGCTCAAGAGAAGCTACTTT

Accordingly, preferably the CV-A6 3C protease polypeptide is encoded by the DNA nucleotide sequence substantially as set out in SEQ ID NO: 172, or a variant or fragment thereof.

One embodiment of codon optimised nucleic acid (DNA) encoding the CV-A6 3C protease polypeptide is provided herein as SEQ ID No: 173, as follows:

[SEQ ID No: 173]
GGCCCTTCTCTGGATTTTGCCCTGAGCCTGCTGCGGCGGAACATCAGACAGGTGCAGACAGATCAGGGCCACTTCACC
ATGCTGGGCGTCAGAGATAGACTGGCCGTGCTGCCTAGACACAGCCAGCCTGGAAAGACCATCTGGGTCGAGCACAAG
CTGGTCAACGTGGTGGATGCCGTGGAACTGGTGGATGAGCAGGGCGTGAACCTGGAACTGACCCTGATCACCCTGGAC
ACCAACGAGAAGTTCCGGGACATCACCAAGTTCATCCCCGAGAACATCAGCGCCGCCTCCGATGCCACACTGGTCATC
AATACCGAGCACATGCCCTCCATGTTCGTGCCTGTGGGAGATGTGGTGCAGTACGGCTTCCTGAACCTGAGCGGCAAG
CCCACACACCGGACCATGATGTACAACTTCCCTACCAAGGCCGGCCAGTGCGGCGGAGTGGTTACATCTGTGGGCAAA
GTGATCGGAATCCACATCGGCGGCAATGGCAGACAGGGCTTTTGTGCCGGCCTGAAGAGAAGCTACTTC

Hence, preferably the RNA construct is encoded by a DNA sequence substantially as set out in SEQ ID No: 173, or a fragment or variant thereof.

In an embodiment, the RNA sequence corresponding to the codon optimised DNA sequence of SEQ ID No: 173 is provided herein as SEQ ID No: 174, as follows:

[SEQ ID No: 174]
GGCCCUUCUCUGGAUUUUGCCCUGAGCCUGCUGCGGCGGAACAUCAGACAGGUGCAGACAGAUCAGGGCCACUUCACC
AUGCUGGGCGUCAGAGAUAGACUGGCCGUGCUGCCUAGACACAGCCAGCCUGGAAAGACCAUCUGGGUCGAGCACAAG
CUGGUCAACGUGGUGGAUGCCGUGGAACUGGUGGAUGAGCAGGGCGUGAACCUGGAACUGACCCUGAUCACCCUGGAC
ACCAACGAGAAGUUCCGGGACAUCACCAAGUUCAUCCCCGAGAACAUCAGCGCCGCCUCCGAUGCCACACUGGUCAUC
AAUACCGAGCACAUGCCCUCCAUGUUCGUGCCUGUGGGAGAUGUGGUGCAGUACGGCUUCCUGAACCUGAGCGGCAAG
CCCACACACCGGACCAUGAUGUACAACUUCCCUACCAAGGCCGGCCAGUGCGGCGGAGUGGUUACAUCUGUGGGCAAA
GUGAUCGGAAUCCACAUCGGCGGCAAUGGCAGACAGGGCUUUUGUGCCGGCCUGAAGAGAAGCUACUUC

Hence, preferably the RNA construct comprises a sequence substantially as set out in SEQ ID No: 174, or a fragment or variant thereof.

In one embodiment, the at least one IIP is EV D-68 3C protease (Q68T42; Genome polyprotein Human enterovirus D68; IRF7 cleavage), or an orthologue thereof (Xiang Z, Liu L, Lei X, Zhou Z, He B, Wang J (2015) 3C protease of enterovirus D68 inhibits cellular defense mediated interferon regulatory factor. J Virol., 90, 3, 1613-2161. doi: 10.1128/JVI.02395-15. Print 2016 Feb. 1). One embodiment of the polypeptide sequence of EV D-68 3C protease is represented herein as SEQ ID No: 175, as follows:

[SEQ ID No: 175]
GPGFDFAQAIMKKNTVIARTEKGEFTMLGVYDRVAVIPTHASVGEIIYINDVETRVLDACALRDLTDTNLEITIVKLD
RNQKFRDIRHFLPRCEDDYNDAVLSVHTSKFPNMYIPVGQVTNYGFLNLGGTPTHRILMYNFPTRAGQCGGVVTTTGK
VIGIHVGGNGAQGFAAMLLHSYFTDTQ

Therefore, preferably the RNA construct comprises a nucleotide sequence which encodes an amino acid sequence substantially as set out in SEQ ID No: 175, or a variant or fragment thereof.

In one embodiment, the EV D-68 3C protease polypeptide is encoded by the DNA nucleotide sequence of SEQ ID No: 176, as follows:

[SEQ ID No: 176]
GGACCAGGATTTGATTTTGCGCAAGCCATAATGAAGAAAAATACTGTTATTGCTAGAACTGAAAAAGGCGAGTTCACA
ATGCTTGGTGTGTATGATAGAGTGGCAGTCATTCCAACACATGCATCTGTTGGAGAAATCATTTACATCAACGATGTA
GAAACCAGAGTTCTAGATGCATGTGCACTTAGAGACTTGACAGACACAAACCTAGAAATAACTATAGTCAAATTGGAT
CGCAATCAAAAATTTAGAGACATCAGACACTTTTTACCCAGATGTGAGGATGATTACAATGATGCTGTGCTTAGTGTA
CATACATCAAAATTCCCTAACATGTACATTCCAGTTGGACAAGTCACTAACTACGGCTTCTTGAACCTGGGCGGCACA
CCAACACATCGGATTTTAATGTATAATTTTCCAACAAGAGCTGGTCAGTGTGGTGGTGTGGTGACAACCACAGGTAAA
GTGATAGGAATACACGTGGGCGGGAATGGAGCTCAGGGATTCGCAGCAATGTTGCTCCACTCTTACTTTACTGATACA
CAA

Accordingly, preferably the EV D-68 3C protease polypeptide is encoded by the DNA nucleotide sequence substantially as set out in SEQ ID NO: 176, or a variant or fragment thereof.

One embodiment of codon optimised nucleic acid (DNA) encoding the EV D-68 3C protease polypeptide is provided herein as SEQ ID No: 177, as follows:

[SEQ ID No: 177]
GGCCCTGGCTTTGATTTTGCCCAGGCCATCATGAAGAAAAACACCGTGATCGCCCGGACCGAGAAGGGCGAGTTTACA
ATGCTGGGCGTGTACGACAGAGTGGCCGTGATTCCTACACACGCCTCTGTGGGCGAGATCATCTACATCAACGACGTG
GAAACCAGAGTGCTGGACGCCTGCGCTCTGAGAGATCTGACCGACACCAACCTGGAAATCACCATCGTGAAGCTGGAC
CGGAACCAGAAGTTCCGGGACATCCGGCACTTTCTGCCCAGATGCGAGGACGACTACAACGACGCTGTGCTGAGCGTG
CACACCAGCAAGTTCCCCAACATGTACATCCCCGTGGGCCAAGTGACCAACTACGGCTTCCTGAATCTCGGCGGCACC
CCTACACACCGGATCCTGATGTACAACTTCCCCACCAGAGCCGGCCAGTGTGGCGGAGTGGTTACCACAACAGGCAAA
GTGATCGGCATCCACGTCGGCGGAAATGGCGCTCAGGGATTTGCTGCCATGCTGCTGCACAGCTACTTCACCGACACA
CAG

Hence, preferably the RNA construct is encoded by a DNA sequence substantially as set out in SEQ ID No: 177, or a fragment or variant thereof.

In an embodiment, the RNA sequence corresponding to the codon optimised DNA sequence of SEQ ID No: 177 is provided herein as SEQ ID No: 178, as follows:

[SEQ ID No: 178]
GGCCCUGGCUUUGAUUUUGCCCAGGCCAUCAUGAAGAAAAACACCGUGAUCGCCCGGACCGAGAAGGGCGAGUUUACA
AUGCUGGGCGUGUACGACAGAGUGGCCGUGAUUCCUACACACGCCUCUGUGGGCGAGAUCAUCUACAUCAACGACGUG
GAAACCAGAGUGCUGGACGCCUGCGCUCUGAGAGAUCUGACCGACACCAACCUGGAAAUCACCAUCGUGAAGCUGGAC
CGGAACCAGAAGUUCCGGGACAUCCGGCACUUUCUGCCCAGAUGCGAGGACGACUACAACGACGCUGUGCUGAGCGUG
CACACCAGCAAGUUCCCCAACAUGUACAUCCCCGUGGGCCAAGUGACCAACUACGGCUUCCUGAAUCUCGGCGGCACC
CCUACACACCGGAUCCUGAUGUACAACUUCCCCACCAGAGCCGGCCAGUGUGGCGGAGUGGUUACCACAACAGGCAAA
GUGAUCGGCAUCCACGUCGGCGGAAAUGGCGCUCAGGGAUUUGCUGCCAUGCUGCUGCACAGCUACUUCACCGACACA
CAG

Hence, preferably the RNA construct comprises a sequence substantially as set out in SEQ ID No: 178, or a fragment or variant thereof.

In one embodiment, the at least one IIP is EVD-71 3C protease (Q0JRV3; Genome polyprotein Human enterovirus 71 Protease 3C), or an orthologue thereof. One embodiment of the polypeptide sequence of EVD-71 3C protease protein is represented herein as SEQ ID No: 179, as follows:

[SEQ ID No: 179]
GPSLDFALSLLRRNIRQVQTDQGHFTMLGVRDHLAVLPRHSQPGKTIWVEHKLVKIVDAVELVDEQGVNLELTLVTLD
TNEKFRDITRFIPETISPASDATLVINTEHMPSMFVPVGDVVQYGFLNLSGKPTHRTMMYNFPTKAGQCGGAVTAVGK
VIGIHIGGNGRQGFCAALKRGYF

Therefore, preferably the RNA construct comprises a nucleotide sequence which encodes an amino acid sequence substantially as set out in SEQ ID No: 179, or a variant or fragment thereof.

In one embodiment, the EVD-71 3C protease polypeptide is encoded by the DNA nucleotide sequence of SEQ ID No: 180, as follows:

[SEQ ID No: 180]
GGGCCGAGCTTGGACTTCGCCCTATCTCTACTTAGGAGGAACATTAGGCAGGTCCAAACCGACCAGGGCCACTTTACA
ATGTTAGGAGTGCGAGACCACTTGGCTGTGCTCCCCAGACACTCCCAACCAGGAAAGACCATCTGGGTTGAACACAAA
TTAGTGAAGATCGTAGACGCTGTGGAGCTAGTAGATGAACAAGGGGTTAACCTAGAGCTCACACTGGTAACGCTTGAC
ACCAACGAAAAATTTAGAGACATCACAAGATTCATACCAGAAACAATTAGTCCTGCTAGTGATGCCACTTTAGTTATA
AATACTGAACATATGCCCAGTATGTTTGTGCCAGTTGGAGATGTGGTCCAGTATGGATTTTTGAACCTTAGTGGTAAG
CCCACTCACAGGACTATGATGTACAATTTCCCAACAAAAGCAGGACAGTGTGGTGGTGCTGTGACTGCCGTAGGTAAA
GTGATTGGGATTCACATTGGTGGCAACGGTAGACAAGGTTTCTGCGCTGCCCTGAAGAGGGGTTACTTT

Accordingly, preferably the EVD-71 3C protease polypeptide is encoded by the DNA nucleotide sequence substantially as set out in SEQ ID NO: 180, or a variant or fragment thereof.

One embodiment of codon optimised nucleic acid (DNA) encoding the EVD-71 3C protease polypeptide is provided herein as SEQ ID No: 181, as follows:

[SEQ ID No: 181]
GGCCCTTCTCTGGATTTTGCCCTGAGCCTGCTGCGGCGGAACATCAGACAGGTGCAGACAGATCAGGGCCACTTCACC
ATGCTGGGCGTCAGAGATCATCTGGCCGTGCTGCCTAGACACAGCCAGCCTGGAAAGACCATCTGGGTCGAGCACAAG
CTGGTCAAGATCGTGGACGCCGTGGAACTGGTGGATGAGCAGGGCGTTAACCTGGAACTGACCCTGGTCACCCTGGAC
ACCAACGAGAAGTTCCGGGACATCACCCGGTTCATCCCCGAGACAATTAGCCCTGCCTCCGACGCCACACTGGTCATC
AATACCGAGCACATGCCCTCCATGTTCGTGCCTGTGGGAGATGTGGTGCAGTACGGCTTCCTGAACCTGAGCGGCAAG
CCCACACACCGGACCATGATGTACAACTTCCCTACCAAGGCCGGCCAGTGCGGCGGAGCTGTTACAGCTGTGGGAAAA
GTGATCGGCATCCACATCGGCGGCAATGGCAGACAGGGATTCTGTGCCGCTCTGAAGAGAGGCTACTTC

Hence, preferably the RNA construct is encoded by a DNA sequence substantially as set out in SEQ ID No: 181, or a fragment or variant thereof.

In an embodiment, the RNA sequence corresponding to the codon optimised DNA sequence of SEQ ID No: 181 is provided herein as SEQ ID No: 182, as follows:

[SEQ ID No: 182]
GGCCCUUCUCUGGAUUUUGCCCUGAGCCUGCUGCGGCGGAACAUCAGACAGGUGCAGACAGAUCAGGGCCACUUCACC
AUGCUGGGCGUCAGAGAUCAUCUGGCCGUGCUGCCUAGACACAGCCAGCCUGGAAAGACCAUCUGGGUCGAGCACAAG
CUGGUCAAGAUCGUGGACGCCGUGGAACUGGUGGAUGAGCAGGGCGUUAACCUGGAACUGACCCUGGUCACCCUGGAC
ACCAACGAGAAGUUCCGGGACAUCACCCGGUUCAUCCCCGAGACAAUUAGCCCUGCCUCCGACGCCACACUGGUCAUC
AAUACCGAGCACAUGCCCUCCAUGUUCGUGCCUGUGGGAGAUGUGGUGCAGUACGGCUUCCUGAACCUGAGCGGCAAG
CCCACACACCGGACCAUGAUGUACAACUUCCCUACCAAGGCCGGCCAGUGCGGCGGAGCUGUUACAGCUGUGGGAAAA
GUGAUCGGCAUCCACAUCGGCGGCAAUGGCAGACAGGGAUUCUGUGCCGCUCUGAAGAGAGGCUACUUC

Hence, preferably the RNA construct comprises a sequence substantially as set out in SEQ ID No: 182, or a fragment or variant thereof.

In one embodiment, the at least one IIP is Polio virus HEV-C 3C protease (P03300; Genome polyprotein Poliovirus type 1 (strain Mahoney) Human Enterovirus), or an orthologue thereof. Lei X, Xiao X, Wang J (2016) Innate Immunity Evasion by Enteroviruses: Insights into Virus-Host Interaction. Viruses 8, 22; doi:10.3390/v8010022. One embodiment of the polypeptide sequence of Polio virus HEV-C 3C protease is represented herein as SEQ ID No: 183, as follows:

[SEQ ID No: 183]
GPGFDYAVAMAKRNIVTATTSKGEFTMLGVHDNVAILPTHASPGESIVIDGKEVEILDAKALEDQAGTNLEITIITLK
RNEKFRDIRPHIPTQITETNDGVLIVNTSKYPNMYVPVGAVTEQGYLNLGGRQTARTLMYNFPTRAGQCGGVITCTGK
VIGMHVGGNGSHGFAAALKRSYFTQSQ

Therefore, preferably the RNA construct comprises a nucleotide sequence which encodes an amino acid sequence substantially as set out in SEQ ID No: 183, or a variant or fragment thereof.

In one embodiment, the Polio virus HEV-C 3C protease polypeptide is encoded by the DNA nucleotide sequence of SEQ ID No: 184, as follows:

[SEQ ID No: 184]
GGACCAGGGTTCGATTACGCAGTGGCTATGGCTAAAAGAAACATTGTTACAGCAACTACTAGCAAGGGAGAGTTCACT
ATGTTAGGAGTCCACGACAACGTGGCTATTTTACCAACCCACGCTTCACCTGGTGAAAGCATTGTGATCGATGGCAAA
GAAGTGGAGATCTTGGATGCCAAAGCGCTCGAAGATCAAGCAGGAACCAATCTTGAAATCACTATAATCACTCTAAAG
AGAAATGAAAAGTTCAGAGACATTAGACCACATATACCTACTCAAATCACTGAGACAAATGATGGAGTCTTGATCGTG
AACACTAGCAAGTACCCCAATATGTATGTTCCTGTCGGTGCTGTGACTGAACAGGGATATCTAAATCTCGGTGGGCGC
CAAACTGCTCGTACTCTAATGTACAACTTTCCAACCAGAGCAGGACAGTGTGGTGGAGTCATCACATGTACTGGGAAA
GTCATCGGGATGCATGTTGGTGGGAACGGTTCACACGGGTTTGCAGCGGCCCTGAAGCGATCATACTTCACTCAGAGT
CAA

Accordingly, preferably the Polio virus HEV-C 3C protease polypeptide is encoded by the DNA nucleotide sequence substantially as set out in SEQ ID NO: 184, or a variant or fragment thereof.

One embodiment of codon optimised nucleic acid (DNA) encoding the Polio virus HEV-C 3C protease polypeptide is provided herein as SEQ ID No: 185, as follows:

[SEQ ID No: 185]
GGCCCTGGCTTTGATTATGCCGTGGCCATGGCTAAGCGGAACATCGTGACAGCCACCACCAGCAAGGGCGAGTTTACA
ATGCTGGGCGTGCACGACAACGTGGCCATCCTGCCTACACATGCTAGCCCTGGCGAGAGCATCGTGATCGACGGCAAA
GAGGTGGAAATCCTGGACGCCAAGGCTCTGGAAGATCAGGCCGGCACCAACCTGGAAATCACCATCATCACCCTGAAG
CGGAACGAGAAGTTCCGGGACATCAGACCTCACATCCCCACACAGATCACCGAGACAAACGACGGCGTGCTGATCGTG
AATACCAGCAAGTACCCCAATATGTACGTGCCCGTGGGCGCCGTGACAGAGCAGGGATATCTGAATCTCGGCGGCAGA
CAGACCGCCAGAACACTGATGTACAACTTCCCCACCAGAGCCGGCCAGTGCGGCGGAGTGATTACATGTACCGGCAAA
GTGATCGGCATGCACGTCGGCGGCAATGGCTCTCACGGATTTGCTGCCGCTCTGAAGAGAAGCTACTTCACCCAGAGC
CAG

Hence, preferably the RNA construct is encoded by a DNA sequence substantially as set out in SEQ ID No: 185, or a fragment or variant thereof.

In an embodiment, the RNA sequence corresponding to the codon optimised DNA sequence of SEQ ID No: 185 is provided herein as SEQ ID No: 186, as follows:

[SEQ ID No: 186]
GGCCCUGGCUUUGAUUAUGCCGUGGCCAUGGCUAAGCGGAACAUCGUGACAGCCACCACCAGCAAGGGCGAGUUUACA
AUGCUGGGCGUGCACGACAACGUGGCCAUCCUGCCUACACAUGCUAGCCCUGGCGAGAGCAUCGUGAUCGACGGCAAA
GAGGUGGAAAUCCUGGACGCCAAGGCUCUGGAAGAUCAGGCCGGCACCAACCUGGAAAUCACCAUCAUCACCCUGAAG
CGGAACGAGAAGUUCCGGGACAUCAGACCUCACAUCCCCACACAGAUCACCGAGACAAACGACGGCGUGCUGAUCGUG
AAUACCAGCAAGUACCCCAAUAUGUACGUGCCCGUGGGCGCCGUGACAGAGCAGGGAUAUCUGAAUCUCGGCGGCAGA
CAGACCGCCAGAACACUGAUGUACAACUUCCCCACCAGAGCCGGCCAGUGCGGCGGAGUGAUUACAUGUACCGGCAAA
GUGAUCGGCAUGCACGUCGGCGGCAAUGGCUCUCACGGAUUUGCUGCCGCUCUGAAGAGAAGCUACUUCACCCAGAGC
CAG

Hence, preferably the RNA construct comprises a sequence substantially as set out in SEQ ID No: 186, or a fragment or variant thereof.

In one embodiment, the at least one IIP is Poliovirus HEV-C 2A protease (P03300; Genome polyprotein Poliovirus type 1 (strain Mahoney) Human Enterovirus), or an orthologue thereof. Feng Q, Langeris M A, Lork M, Nguyen M, Hato S V, Lanke K, Endad L, Bhoopathi P, Fisher P B, Lloyd R E, van Kuppeveld F J M (2014) Enterovirus 2Apro targets MDA5 and MAVS in infected cells. J Virol., 88, 6, 3369-3377. One embodiment of the polypeptide sequence of Poliovirus HEV-C 2A protease is represented herein as SEQ ID No: 187, as follows:

[SEQ ID No: 187]
GFGHQNKAVYTAGYKICNYHLATQDDLQNAVNVMWSRDLLVTESRAQGTDSIARCNCNAGVYYCESRRKYYPVSFVGP
TFQYMEANNYYPARYQSHMLIGHGFASPGDCGGILRCHHGVIGIITAGGEGLVAFSDIRDLYAYEEEAMEQ

Therefore, preferably the RNA construct comprises a nucleotide sequence which encodes an amino acid sequence substantially as set out in SEQ ID No: 187, or a variant or fragment thereof.

In one embodiment, the Poliovirus HEV-C 2A protease polypeptide is encoded by the DNA nucleotide sequence of SEQ ID No: 188, as follows:

[SEQ ID No: 188]
GGATTCGGACACCAAAACAAAGCGGTGTACACTGCAGGTTACAAAATTTGCAACTACCACTTGGCCACTCAGGATGAT
TTGCAAAACGCAGTGAACGTCATGTGGAGTAGAGACCTCTTAGTCACAGAATCAAGAGCCCAGGGCACCGATTCAATC
GCAAGGTGCAATTGCAACGCAGGGGTGTACTACTGCGAGTCTAGAAGGAAATACTACCCAGTATCCTTCGTTGGCCCA
ACGTTCCAGTACATGGAGGCTAATAACTATTACCCAGCTAGGTACCAGTCCCATATGCTCATTGGCCATGGATTCGCA
TCTCCAGGGGATTGTGGTGGCATACTCAGATGTCACCACGGGGTGATAGGGATCATTACTGCTGGTGGCGAAGGGTTG
GTTGCATTTTCAGACATTAGAGACTTGTATGCCTACGAAGAAGAAGCCATGGAACAA

Accordingly, preferably the Poliovirus HEV-C 2A protease polypeptide is encoded by the DNA nucleotide sequence substantially as set out in SEQ ID NO: 188, or a variant or fragment thereof.

One embodiment of codon optimised nucleic acid (DNA) encoding the Poliovirus HEV-C 2A protease polypeptide is provided herein as SEQ ID No: 189, as follows:

[SEQ ID No: 189]
GGCTTTGGCCACCAGAACAAGGCCGTGTACACAGCCGGCTACAAGATCTGCAACTACCACCTGGCCACACAGGACGAC
CTGCAGAACGCCGTGAATGTGATGTGGTCCAGGGACCTGCTGGTCACCGAATCTAGAGCCCAGGGCACCGACTCTATC
GCCAGATGCAACTGTAATGCCGGCGTGTACTACTGCGAGAGCCGGCGGAAGTACTACCCCGTGTCTTTTGTGGGCCCC
ACCTTCCAGTACATGGAAGCCAACAACTACTACCCTGCCAGATACCAGAGCCACATGCTGATCGGCCACGGCTTTGCT
AGCCCTGGCGATTGTGGCGGCATCCTGAGATGTCACCATGGCGTGATCGGCATCATCACCGCTGGCGGAGAAGGACTG
GTGGCCTTCAGCGACATCAGAGATCTGTACGCCTACGAAGAGGAAGCCATGGAACAG

Hence, preferably the RNA construct is encoded by a DNA sequence substantially as set out in SEQ ID No: 189, or a fragment or variant thereof.

In an embodiment, the RNA sequence corresponding to the codon optimised DNA sequence of SEQ ID No: 189 is provided herein as SEQ ID No: 190, as follows:

[SEQ ID No: 190]
GGCUUUGGCCACCAGAACAAGGCCGUGUACACAGCCGGCUACAAGAUCUGCAACUACCACCUGGCCACACAGGACGAC
CUGCAGAACGCCGUGAAUGUGAUGUGGUCCAGGGACCUGCUGGUCACCGAAUCUAGAGCCCAGGGCACCGACUCUAUC
GCCAGAUGCAACUGUAAUGCCGGCGUGUACUACUGCGAGAGCCGGCGGAAGUACUACCCCGUGUCUUUUGUGGGCCCC
ACCUUCCAGUACAUGGAAGCCAACAACUACUACCCUGCCAGAUACCAGAGCCACAUGCUGAUCGGCCACGGCUUUGCU
AGCCCUGGCGAUUGUGGCGGCAUCCUGAGAUGUCACCAUGGCGUGAUCGGCAUCAUCACCGCUGGCGGAGAAGGACUG
GUGGCCUUCAGCGACAUCAGAGAUCUGUACGCCUACGAAGAGGAAGCCAUGGAACAG

Hence, preferably the RNA construct comprises a sequence substantially as set out in SEQ ID No: 190, or a fragment or variant thereof.

In one embodiment, the at least one IIP is CVB3 2A protease (P03313; Genome polyprotein Coxsackievirus B3 (strain Nancy)), or an orthologue thereof. One embodiment of the polypeptide sequence of CVB3 2A protease is represented herein as SEQ ID No: 191, as follows:

[SEQ ID No: 191]
GAFGQQSGAVYVGNYRVVNRHLATSADWQNCVWESYNRDLLVSTTTAHGCDIIARCQCTTGVYFCASKNKHYPISFEG
PGLVEVQESEYYPRRYQSHVLLAAGFSEPGDCGGILRCEHGVIGIVTMGGEGVVGFADIRDLLWLEDDAMEQ

Therefore, preferably the RNA construct comprises a nucleotide sequence which encodes an amino acid sequence substantially as set out in SEQ ID No: 191, or a variant or fragment thereof.

In one embodiment, the CVB3 2A protease polypeptide is encoded by the DNA nucleotide sequence of SEQ ID No: 192, as follows:

[SEQ ID No: 192]
GGCGCATTTGGACAACAATCAGGGGCAGTGTATGTGGGGAACTACAGGGTGGTAAATAGACATCTAGCTACCAGTGCT
GACTGGCAAAACTGTGTGTGGGAAAGTTACAACAGAGACCTCTTAGTGAGCACGACCACAGCACATGGATGTGATATT
ATAGCCAGATGICAGTGCACAACGGGAGTGTACTTTTGTGCGTCCAAAAACAAGCACTACCCAATTTCGTTTGAAGGA
CCAGGTCTAGTAGAGGTCCAAGAGAGTGAATACTACCCCAGGAGATACCAATCCCATGTGCTTTTAGCAGCTGGATTT
TCCGAACCAGGIGACTGIGGCGGTATCCTAAGGIGTGAGCATGGTGTCATTGGCATTGTGACCATGGGGGGTGAAGGC
GTGGTCGGCTTTGCAGACATCCGTGATCTCCTGTGGCTGGAAGATGATGCAATGGAACAG

Accordingly, preferably the CVB3 2A protease polypeptide is encoded by the DNA nucleotide sequence substantially as set out in SEQ ID NO: 192, or a variant or fragment thereof.

One embodiment of codon optimised nucleic acid (DNA) encoding the CVB3 2A protease polypeptide is provided herein as SEQ ID No: 193, as follows:

[SEQ ID No: 193]
GGAGCTTTTGGACAGCAGTCTGGCGCCGTGTACGTGGGCAATTACCGGGTCGTGAATAGACACCTGGCCACCTCTGCC
GACTGGCAGAATTGTGTGTGGGAGAGCTACAACCGGGACCTGCTGGTGTCTACCACAACAGCCCACGGCTGCGACATC
ATTGCCAGATGCCAGTGTACAACCGGCGTGTACTTCTGCGCCAGCAAGAACAAGCACTACCCCATCAGCTTCGAAGGC
CCTGGCCTGGTGGAAGTGCAAGAGAGCGAGTACTACCCTCGGAGATACCAGAGCCACGTGCTGCTGGCCGCTGGCTTT
TCTGAACCTGGCGATTGTGGCGGCATCCTGAGATGTGAACACGGCGTGATCGGCATCGTGACCATGGGCGGAGAAGGC
GTTGTGGGCTTCGCCGACATTAGAGATCTGCTGTGGCTGGAAGATGACGCCATGGAACAG

Hence, preferably the RNA construct is encoded by a DNA sequence substantially as set out in SEQ ID No: 193, or a fragment or variant thereof.

In an embodiment, the RNA sequence corresponding to the codon optimised DNA sequence of SEQ ID No: 193 is provided herein as SEQ ID No: 194, as follows:

[SEQ ID No: 194]
GGAGCUUUUGGACAGCAGUCUGGCGCCGUGUACGUGGGCAAUUACCGGGUCGUGAAUAGACACCUGGCCACCUCUGCC
GACUGGCAGAAUUGUGUGUGGGAGAGCUACAACCGGGACCUGCUGGUGUCUACCACAACAGCCCACGGCUGCGACAUC
AUUGCCAGAUGCCAGUGUACAACCGGCGUGUACUUCUGCGCCAGCAAGAACAAGCACUACCCCAUCAGCUUCGAAGGC
CCUGGCCUGGUGGAAGUGCAAGAGAGCGAGUACUACCCUCGGAGAUACCAGAGCCACGUGCUGCUGGCCGCUGGCUUU
UCUGAACCUGGCGAUUGUGGCGGCAUCCUGAGAUGUGAACACGGCGUGAUCGGCAUCGUGACCAUGGGCGGAGAAGGC
GUUGUGGGCUUCGCCGACAUUAGAGAUCUGCUGUGGCUGGAAGAUGACGCCAUGGAACAG

Hence, preferably the RNA construct comprises a sequence substantially as set out in SEQ ID No: 194, or a fragment or variant thereof.

In one embodiment, the at least one IIP is EV71 2A protease (B9VUU3; Genome polyprotein Human enterovirus 71), or an orthologue thereof. One embodiment of the polypeptide sequence of EV71 2A protease is represented herein as SEQ ID No: 195, as follows:

[SEQ ID No: 195]
GKFGQQSGAIYVGNFRVVNRHLATHNDWANLVWEDSSRDLLVSSTTAQGCDTIARCNCQTGVYYCNSRRKHYPVSFSK
PSLIYVEASEYYPARYQSHLMLAQGHSEPGDCGGILRCQHGVVGIVSTGGNGLVGFADVRDLLWLDEEAMEQ

Therefore, preferably the RNA construct comprises a nucleotide sequence which encodes an amino acid sequence substantially as set out in SEQ ID No: 195, or a variant or fragment thereof.

In one embodiment, the EV71 2A protease polypeptide is encoded by the DNA nucleotide sequence of SEQ ID No: 196, as follows:

[SEQ ID No: 196]
GGAAAATTTGGGCAACAGTCTGGGGCCATTTATGTGGGTAACTTTAGAGT
GGTCAACCGTCATCTTGCCACTCACAATGATTGGGCAAATCTTGTTTGGG
AAGACAGCTCTCGCGACTTACTCGTGTCATCCACCACCGCCCAAGGTTGT
GACACGATTGCCCGCTGCAATTGCCAGACAGGGGTGTACTACTGTAACTC
GAGGAGAAAACACTACCCAGTCAGTTTTTCAAAACCCAGTCTGATCTATG
TAGAGGCTAGCGAGTATTACCCAGCCAGGTACCAGTCACATCTTATGCTC
GCACAGGGCCACTCAGAGCCTGGTGATTGCGGTGGTATCCTTAGATGCCA
ACATGGCGTCGTCGGTATAGTGTCAACTGGTGGTAACGGGCTCGTTGGCT
TTGCAGACGTCAGGGACCTCTTGTGGTTAGATGAAGAAGCTATGGAGCAG

Accordingly, preferably the EV71 2A protease polypeptide is encoded by the DNA nucleotide sequence substantially as set out in SEQ ID NO: 196, or a variant or fragment thereof.

One embodiment of codon optimised nucleic acid (DNA) encoding the EV71 2A protease polypeptide is provided herein as SEQ ID No: 197, as follows:

[SEQ ID No: 197]
GGCAAGTTTGGACAGCAGAGCGGCGCCATCTACGTGGGCAATTTCCGGGT
CGTGAACCGGCACCTGGCCACACATAACGACTGGGCCAATCTCGTGTGGG
AAGATAGCAGCAGGGACCTGCTGGTGTCCAGCACAACAGCCCAGGGCTGC
GATACAATCGCCAGATGCAATTGCCAGACCGGCGTGTACTACTGCAACAG
CAGACGGAAGCACTACCCCGTGTCCTTCAGCAAGCCCAGCCTGATCTATG
TGGAAGCCAGCGAGTACTACCCCGCCAGATACCAGTCTCACCTGATGCTG
GCCCAGGGCCATTCTGAGCCAGGCGATTGTGGCGGAATCCTGAGATGCCA
GCATGGCGTCGTGGGCATTGTGTCTACCGGCGGAAATGGCCTCGTGGGAT
TTGCCGATGTTCGCGACCTGCTGTGGCTGGACGAAGAGGCTATGGAACAG

Hence, preferably the RNA construct is encoded by a DNA sequence substantially as set out in SEQ ID No: 197, or a fragment or variant thereof.

In an embodiment, the RNA sequence corresponding to the codon optimised DNA sequence of SEQ ID No: 197 is provided herein as SEQ ID No: 198, as follows:

[SEQ ID No: 198]
GGCAAGUUUGGACAGCAGAGCGGCGCCAUCUACGUGGGCAAUUUCCGGGU
CGUGAACCGGCACCUGGCCACACAUAACGACUGGGCCAAUCUCGUGUGGG
AAGAUAGCAGCAGGGACCUGCUGGUGUCCAGCACAACAGCCCAGGGCUGC
GAUACAAUCGCCAGAUGCAAUUGCCAGACCGGCGUGUACUACUGCAACAG
CAGACGGAAGCACUACCCCGUGUCCUUCAGCAAGCCCAGCCUGAUCUAUG
UGGAAGCCAGCGAGUACUACCCCGCCAGAUACCAGUCUCACCUGAUGCUG
GCCCAGGGCCAUUCUGAGCCAGGCGAUUGUGGCGGAAUCCUGAGAUGCCA
GCAUGGCGUCGUGGGCAUUGUGUCUACCGGCGGAAAUGGCCUCGUGGGAU
UUGCCGAUGUUCGCGACCUGCUGUGGCUGGACGAAGAGGCUAUGGAACAG

Hence, preferably the RNA construct comprises a sequence substantially as set out in SEQ ID No: 198, or a fragment or variant thereof.

In one embodiment, the at least one IIP is hMPV G protein (Q6WB94; Major surface glycoprotein G Human metapneumovirus (strain CAN97-83)), or an orthologue thereof. Bao X, Liu T, Shan Y, Li K, Garofolo R P, Casola A (2008) Human Metapneumovirus Glycoprotein G Inhibits Innate Immune Responses. PLOS Pathogens, 4, 5, e1000077. One embodiment of the polypeptide sequence of hMPV G protein is represented herein as SEQ ID No: 199, as follows:

[SEQ ID No: 199]
MEVKVENIRAIDMLKARVKNRVARSKCFKNASLILIGITTLSIALNIYLI
INYTIQKTSSESEHHTSSPPTESNKEASTISTDNPDINPNSQHPTQQSTE
NPTLNPAASVSPSETEPASTPDTTNRLSSVDRSTAQPSESRTKTKPTVHT
RNNPSTASSTQSPPRATTKAIRRATTFRMSSTGKRPTTTSVQSDSSTTTQ
NHEETGSANPQASVSTMQN

Therefore, preferably the RNA construct comprises a nucleotide sequence which encodes an amino acid sequence substantially as set out in SEQ ID No: 199, or a variant or fragment thereof.

In one embodiment, the hMPV G polypeptide is encoded by the DNA nucleotide sequence of SEQ ID No: 200, as follows:

[SEQ ID No: 200]
ATGGAGGTGAAAGTAGAGAACATTCGAGCAATAGACATGCTCAAAGCAAG
AGTGAAAAATCGTGTGGCACGTAGCAAATGCTTTAAAAATGCTTCTTTAA
TCCTCATAGGAATAACTACACTGAGTATAGCTCTCAATATCTATCTGATC
ATAAACTACACAATACAAAAAACCTCATCTGAATCAGAACACCACACCAG
CTCACCACCCACAGAATCCAACAAGGAAGCTTCAACAATCTCCACAGACA
ACCCAGACATCAATCCAAACTCACAGCATCCAACTCAACAGTCCACAGAA
AACCCCACACTCAACCCCGCAGCATCAGTGAGCCCATCAGAAACAGAACC
AGCATCAACACCAGACACAACAAACCGCCTGTCCTCCGTAGACAGGTCCA
CAGCACAACCAAGTGAAAGCAGAACAAAGACAAAACCGACAGTCCACACA
AGAAACAACCCAAGCACAGCTTCCAGTACACAATCCCCACCACGGGCAAC
AACGAAGGCAATCCGCAGAGCCACCACTTTCCGCATGAGCAGCACAGGAA
AAAGACCAACCACAACATCAGTCCAGTCCGACAGCAGCACCACAACCCAA
AATCATGAAGAAACAGGTTCAGCGAACCCACAGGCATCTGTAAGCACAAT
GCAAAAC

Accordingly, preferably the hMPV G polypeptide is encoded by the DNA nucleotide sequence substantially as set out in SEQ ID NO: 200, or a variant or fragment thereof.

One embodiment of codon optimised nucleic acid (DNA) encoding the hMPV G polypeptide is provided herein as SEQ ID No: 201, as follows:

[SEQ ID No: 201]
ATGGAAGTGAAGGTCGAGAACATCCGGGCCATCGACATGCTGAAGGCCAG
AGTGAAGAACAGAGTGGCCCGGTCCAAGTGCTTCAAGAACGCCAGCCTGA
TCCTGATCGGCATCACCACACTGTCTATCGCCCTGAACATCTACCTGATC
ATCAACTACACCATCCAGAAAACCAGCAGCGAGAGCGAGCACCACACAAG
CTCTCCACCTACCGAGAGCAACAAAGAGGCCAGCACCATCAGCACCGACA
ATCCCGACATCAACCCCAACTCTCAGCACCCCACACAGCAGTCCACCGAG
AATCCCACACTGAACCCTGCCGCCTCTGTGTCCCCATCTGAGACAGAACC
TGCCAGCACACCCGACACCACCAACAGACTGTCTAGCGTGGACAGAAGCA
CAGCCCAGCCTAGCGAGAGCCGGACCAAGACAAAACCTACCGTGCACACC
CGGAACAACCCTAGCACAGCCAGCTCTACACAGAGCCCTCCAAGAGCCAC
CACCAAGGCCATTAGAAGGGCCACCACCTTCCGGATGAGCAGCACCGGCA
AAAGACCTACCACCACCAGCGTGCAGAGCGACAGCAGCACAACCACACAG
AACCACGAGGAAACCGGCAGCGCCAATCCTCAGGCTAGCGTGTCCACCAT
GCAGAAC

Hence, preferably the RNA construct is encoded by a DNA sequence substantially as set out in SEQ ID No: 201, or a fragment or variant thereof.

In an embodiment, the RNA sequence corresponding to the codon optimised DNA sequence of SEQ ID No: 201 is provided herein as SEQ ID No: 202, as follows:

[SEQ ID No: 202]
AUGGAAGUGAAGGUCGAGAACAUCCGGGCCAUCGACAUGCUGAAGGCCAG
AGUGAAGAACAGAGUGGCCCGGUCCAAGUGCUUCAAGAACGCCAGCCUGA
UCCUGAUCGGCAUCACCACACUGUCUAUCGCCCUGAACAUCUACCUGAUC
AUCAACUACACCAUCCAGAAAACCAGCAGCGAGAGCGAGCACCACACAAG
CUCUCCACCUACCGAGAGCAACAAAGAGGCCAGCACCAUCAGCACCGACA
AUCCCGACAUCAACCCCAACUCUCAGCACCCCACACAGCAGUCCACCGAG
AAUCCCACACUGAACCCUGCCGCCUCUGUGUCCCCAUCUGAGACAGAACC
UGCCAGCACACCCGACACCACCAACAGACUGUCUAGCGUGGACAGAAGCA
CAGCCCAGCCUAGCGAGAGCCGGACCAAGACAAAACCUACCGUGCACACC
CGGAACAACCCUAGCACAGCCAGCUCUACACAGAGCCCUCCAAGAGCCAC
CACCAAGGCCAUUAGAAGGGCCACCACCUUCCGGAUGAGCAGCACCGGCA
AAAGACCUACCACCACCAGCGUGCAGAGCGACAGCAGCACAACCACACAG
AACCACGAGGAAACCGGCAGCGCCAAUCCUCAGGCUAGCGUGUCCACCAU
GCAGAAC

Hence, preferably the RNA construct comprises a sequence substantially as set out in SEQ ID No: 202, or a fragment or variant thereof.

The following viral IIPs are believed to cause cleavage of RIG-I.

In one embodiment, the at least one IIP is CVB3 3C protease (P03313; Genome polyprotein Coxsackievirus B3 (strain Nancy)), or an orthologue thereof. Xiao H, Li J, Yang X, Li Z, Wang Y, Rui Y, Liu R, Zhang W (2021) Ectopic Expression of TRIM25 Restores RIG-I Expression and IFN Production Reduced by Multiple Enteroviruses 3Cpro. Virol Sin: 1-12.doi: 10.1007/s12250-021-00410-x. One embodiment of the polypeptide sequence of CVB3 3C protease is represented herein as SEQ ID No: 203, as follows:

[SEQ ID No: 203]
GPAFEFAVAMMKRNSSTVKTEYGEFTMLGIYDRWAVLPRHAKPGPTILMN
DQEVGVLDAKELVDKDGTNLELTLLKLNRNEKFRDIRGFLAKEEVEVNEA
VLAINTSKFPNMYIPVGQVTEYGFLNLGGTPTKRMLMYNFPTRAGQCGGV
LMSTGKVLGIHVGGNGHQGFSAALLKHYFNDEQ

Therefore, preferably the RNA construct comprises a nucleotide sequence which encodes an amino acid sequence substantially as set out in SEQ ID No: 203, or a variant or fragment thereof.

In one embodiment, the CVB3 3C protease polypeptide is encoded by the DNA nucleotide sequence of SEQ ID No: 204, as follows:

[SEQ ID No: 204]
GGCCCTGCCTTTGAGTTCGCCGTCGCAATGATGAAAAGGAACTCAAGCAC
GGTGAAAACTGAATATGGCGAGTTTACCATGCTGGGCATCTATGACAGGT
GGGCCGTTTTGCCACGCCACGCCAAACCTGGGCCAACCATCTTGATGAAT
GATCAAGAGGTTGGTGTGCTAGATGCCAAGGAGCTAGTAGACAAGGACGG
CACCAACTTAGAACTGACACTACTCAAATTGAACCGGAATGAGAAGTTCA
GAGACATCAGAGGCTTCTTAGCCAAGGAGGAAGTGGAGGTTAATGAGGCA
GTGCTAGCAATTAACACCAGCAAGTTTCCCAACATGTACATTCCAGTAGG
ACAGGTCACAGAATACGGCTTCCTAAACCTAGGTGGCACACCCACCAAGA
GAATGCTTATGTACAACTTCCCCACAAGAGCAGGCCAGTGTGGTGGAGTG
CTCATGTCCACCGGCAAGGTACTGGGTATCCATGTTGGTGGAAATGGCCA
TCAGGGCTTCTCAGCAGCACTCCTCAAACACTACTTCAATGATGAGCAA

Accordingly, preferably the CVB3 3C protease polypeptide is encoded by the DNA nucleotide sequence substantially as set out in SEQ ID NO: 204, or a variant or fragment thereof.

One embodiment of codon optimised nucleic acid (DNA) encoding the CVB3 3C protease polypeptide is provided herein as SEQ ID No: 205, as follows:

[SEQ ID No: 205]
GGACCTGCCTTTGAATTCGCCGTGGCCATGATGAAGCGGAACAGCAGCAC
CGTGAAAACCGAGTACGGCGAGTTCACCATGCTGGGCATCTACGACAGAT
GGGCCGTGCTGCCTAGACACGCCAAACCTGGACCTACCATCCTGATGAAC
GACCAAGAAGTGGGCGTTCTGGACGCCAAAGAACTGGTGGACAAGGACGG
CACCAACCTGGAACTGACCCTGCTGAAGCTGAACCGGAACGAGAAGTTCC
GGGATATCAGAGGCTTCCTGGCCAAAGAAGAGGTGGAAGTCAACGAAGCC
GTGCTGGCCATCAACACCAGCAAGTTCCCCAACATGTACATCCCCGTGGG
CCAAGTGACAGAGTACGGCTTCCTGAATCTCGGCGGCACCCCTACCAAGC
GGATGCTGATGTACAACTTCCCCACCAGAGCCGGCCAGTGTGGCGGAGTT
CTTATGTCTACCGGCAAGGTGCTGGGAATCCACGTTGGCGGAAATGGCCA
CCAGGGCTTTTCTGCCGCTCTGCTGAAACACTACTTCAACGACGAGCAG

Hence, preferably the RNA construct is encoded by a DNA sequence substantially as set out in SEQ ID No: 205, or a fragment or variant thereof.

In an embodiment, the RNA sequence corresponding to the codon optimised DNA sequence of SEQ ID No: 205 is provided herein as SEQ ID No: 206, as follows:

[SEQ ID No: 206]
GGACCUGCCUUUGAAUUCGCCGUGGCCAUGAUGAAGCGGAACAGCAGCAC
CGUGAAAACCGAGUACGGCGAGUUCACCAUGCUGGGCAUCUACGACAGAU
GGGCCGUGCUGCCUAGACACGCCAAACCUGGACCUACCAUCCUGAUGAAC
GACCAAGAAGUGGGCGUUCUGGACGCCAAAGAACUGGUGGACAAGGACGG
CACCAACCUGGAACUGACCCUGCUGAAGCUGAACCGGAACGAGAAGUUCC
GGGAUAUCAGAGGCUUCCUGGCCAAAGAAGAGGUGGAAGUCAACGAAGCC
GUGCUGGCCAUCAACACCAGCAAGUUCCCCAACAUGUACAUCCCCGUGGG
CCAAGUGACAGAGUACGGCUUCCUGAAUCUCGGCGGCACCCCUACCAAGC
GGAUGCUGAUGUACAACUUCCCCACCAGAGCCGGCCAGUGUGGCGGAGUU
CUUAUGUCUACCGGCAAGGUGCUGGGAAUCCACGUUGGCGGAAAUGGCCA
CCAGGGCUUUUCUGCCGCUCUGCUGAAACACUACUUCAACGACGAGCAG

Hence, preferably the RNA construct comprises a sequence substantially as set out in SEQ ID No: 206, or a fragment or variant thereof.

In one embodiment, the at least one IIP is Hepatitis C NS3/4A (039929; Genome polyprotein Hepatitis C virus genotype 4a (isolate ED43)), or an orthologue thereof. One embodiment of the polypeptide sequence of Hepatitis C NS3/4A is represented herein as SEQ ID No: 207, as follows:

[SEQ ID No: 207]
APITAYAQQTRGLFSTIVTSLTGRDTNENCGEVQVLSTATQSFLGTAVNG
VMWTVYHGAGAKTISGPKGPVNQMYTNVDQDLVGWPAPPGVRSLAPCTCG
SADLYLVTRHADVIPVRRRGDTRGALLSPRPISILKGSSGGPLLCPMGHR
AGIFRAAVCTRGVAKAVDFVPVESLETTMRSPVFTDNSTPPAVPQTYQVA
HLHAPTGSGKSTKVPAAHAAQGYKVLVLNPSVAATLGFGVYMSKAYGIDP
NIRSGVRTITTGAPITYSTYGKFLADGGCSGGAYDIIICDECYSTDSTTI
LGIGTVLDQAETAGVRLTVLATATPPGSVTTPHSNIEEVALPTTGEIPFY
GKAIPLELIKGGRHLIFCHSKKKCDELARQLTSLGLNAVAYYRGLDVSVI
PTSGDVVVCATDALMTGFTGDFDSVIDCNTSVIQTVDFSLDPTFSIEITT
VPQDAVSRSQRRGRTGRGRLGTYRYVTPGERPSGMFDTAELCECYDAGCA
WYELTPAETTTRLKAYFDTPGLPVCQDHLEFWESVFTGLTHIDGHFLSQT
KQSGENFPYLVAYQATVSAKVWLAPPSWDTMWKCLIRLKPTLHGPTPLLY
RLGSVQNEVVLTHPITKYIMACMSADLEVVT

Therefore, preferably the RNA construct comprises a nucleotide sequence which encodes an amino acid sequence substantially as set out in SEQ ID No: 207, or a variant or fragment thereof.

In one embodiment, the Hepatitis C NS3/4A polypeptide is encoded by the DNA nucleotide sequence of SEQ ID No: 208, as follows:

[SEQ ID No: 208]
GCCCCCATCACAGCATACGCaCAGCAGACCCGCGGCTTGTTCAGCACCAT
CGTAACGAGCCTCACTGGCAGGGACACCAATGAGAATTGTGGCGAAGTGC
AGGTCTTATCCACCGCTACGCAGTCCTTCCTGGGTACTGCGGTTAACGGC
GTGATGTGGACCGTCTACCACGGGGCGGGTGCCAAGACCATCAGCGGCCC
GAAGGGACCTGTCAATCAAATGTACACTAATGTTGACCAAGACTTGGTGG
GGTGGCCAGCACCCCCCGGAGTCAGATCTCTTGCTCCGTGCACCTGCGGC
TCGGCAGACTTGTATCTAGTCACCAGGCACGCGGATGTAATACCCGTGCG
CAGGAGAGGAGACACCAGAGGAGCTCTCTTGAGCCCTAGACCAATATCCA
TTCTTAAGGGATCTTCCGGAGGTCCGCTGCTGTGCCCCATGGGACACCGC
GCCGGCATATTCCGTGCGGCGGTGTGTACTCGGGGGGTAGCCAAGGCGGT
AGACTTCGTCCCGGTTGAATCTCTTGAGACTACCATGAGATCACCAGTGT
TCACTGACAACTCAACACCCCCAGCAGTGCCCCAGACCTACCAGGTCGCG
CACCTACACGCACCAACAGGAAGTGGCAAGAGCACTAAGGTCCCGGCGGC
GCATGCTGCCCAAGGCTATAAAGTGCTAGTGCTCAATCCTTCGGTTGCGG
CCACACTGGGTTTTGGGGTATACATGTCCAAGGCATATGGCATCGACCCG
AACATCCGGTCGGGAGTCAGGACCATCACCACGGGTGCGCCAATCACGTA
CTCAACGTATGGTAAGTTCCTGGCTGATGGAGGTTGCAGCGGAGGGGCAT
ACGACATAATCATCTGTGACGAGTGCTATTCCACTGACTCCACAACGATC
CTTGGCATAGGCACAGTCCTGGACCAAGCGGAGACCGCTGGAGTGCGCCT
CACCGTGCTCGCGACTGCTACTCCGCCAGGGTCAGTGACTACACCTCATT
CCAACATAGAGGAGGTCGCCCTGCCAACAACGGGGGAAATACCCTTTTAC
GGCAAGGCGATCCCTCTGGAGCTGATCAAGGGGGGCAGACATCTCATCTT
CTGCCATTCAAAGAAAAAGTGCGATGAACTGGCCAGACAACTGACATCTC
TTGGTCTGAATGCCGTAGCCTACTACAGAGGCTTAGACGTTTCGGTGATT
CCCACGTCTGGGGACGTCGTGGTATGCGCCACGGACGCCCTCATGACGGG
TTTCACCGGCGACTTTGACTCAGTGATAGACTGCAATACATCTGTGATAC
AGACTGTTGACTTCAGCTTGGACCCCACCTTCTCCATAGAGATTACAACC
GTTCCCCAGGACGCGGTATCCCGCAGCCAGCGGAGAGGCCGCACTGGTAG
GGGGAGGTTGGGCACATACCGGTATGTCACCCCGGGAGAGAGACCATCAG
GCATGTTTGACACTGCAGAGCTTTGCGAGTGCTACGATGCCGGGTGCGCC
TGGTACGAGCTGACACCTGCTGAAACCACAACAAGGCTGAAAGCTTACTT
CGACACACCAGGCCTTCCTGTGTGCCAAGACCATCTGGAATTCTGGGAGA
GCGTCTTTACAGGGTTAACCCACATAGACGGTCATTTCCTATCCCAGACC
AAGCAATCGGGTGAGAATTTCCCGTATCTTGTTGCTTACCAAGCGACGGT
GTCGGCCAAGGTCTGGCTCGCTCCACCAAGCTGGGACACCATGTGGAAGT
GCCTAATTCGCCTTAAGCCCACCCTGCACGGGCCCACGCCCCTCCTCTAC
AGACTGGGGTCTGTGCAGAATGAAGTGGTGCTCACCCATCCCATCACCAA
ATACATCATGGCTTGCATGTCAGCTGATCTCGAGGTAGTGACA

Accordingly, preferably the Hepatitis C NS3/4A polypeptide is encoded by the DNA nucleotide sequence substantially as set out in SEQ ID NO: 208, or a variant or fragment thereof.

One embodiment of codon optimised nucleic acid (DNA) encoding the Hepatitis C NS3/4A polypeptide is provided herein as SEQ ID No: 209, as follows:

[SEQ ID No: 209]
GCCCCTATCACAGCCTACGCTCAGCAGACCAGAGGCCTGTTCAGCACCAT
CGTGACAAGCCTGACCGGCAGAGACACCAACGAGAATTGTGGCGAGGTGC
AGGTCCTGTCTACAGCTACCCAGAGCTTTCTGGGCACCGCCGTGAATGGC
GTGATGTGGACAGTGTATCATGGCGCTGGCGCCAAGACAATCTCTGGCCC
TAAGGGCCCCGTGAACCAGATGTACACCAACGTGGACCAGGACCTCGTTG
GCTGGCCTGCTCCTCCTGGTGTTAGAAGCCTGGCTCCTTGTACATGCGGC
AGCGCCGATCTGTACCTGGTCACAAGACACGCCGACGTGATCCCCGTCAG
AAGAAGAGGCGATACAAGAGGCGCCCTGCTGAGCCCTAGACCTATCTCTA
TCCTGAAGGGCAGCTCTGGCGGCCCTCTGCTTTGTCCTATGGGACACAGA
GCCGGCATCTTCAGAGCCGCCGTGTGTACTAGAGGCGTGGCCAAGGCTGT
GGACTTCGTGCCTGTGGAAAGCCTGGAAACCACCATGAGAAGCCCCGTGT
TCACCGACAACAGCACCCCTCCAGCTGTGCCTCAGACATACCAGGTGGCC
CATCTGCATGCCCCTACAGGCTCTGGCAAGAGCACAAAAGTGCCTGCCGC
TCATGCTGCCCAGGGCTATAAGGTGCTGGTGCTCAATCCTAGCGTGGCCG
CCACACTCGGCTTTGGCGTGTACATGTCTAAGGCCTACGGCATCGACCCC
AACATCAGATCTGGCGTGCGGACCATCACAACAGGCGCCCCAATCACCTA
CTCTACCTACGGCAAGTTCCTGGCCGATGGCGGATGTTCTGGCGGAGCCT
ACGACATCATCATCTGCGACGAGTGCTACAGCACCGACAGCACCACAATC
CTCGGCATCGGCACAGTGCTGGATCAGGCTGAAACAGCCGGCGTCAGACT
GACTGTGCTGGCCACAGCTACACCTCCAGGCAGCGTGACAACCCCTCACA
GCAACATCGAGGAAGTGGCCCTGCCTACAACCGGCGAGATCCCATTCTAT
GGCAAGGCCATTCCTCTCGAGCTGATCAAAGGCGGCAGACACCTGATCTT
TTGCCACAGCAAGAAGAAGTGCGACGAGCTGGCCAGACAGCTGACATCCC
TGGGACTGAATGCCGTGGCCTACTACAGAGGACTGGACGTGTCCGTGATT
CCCACATCTGGCGACGTGGTCGTGTGTGCCACAGATGCCCTGATGACCGG
CTTCACCGGCGACTTCGATAGCGTGATCGACTGCAACACCAGCGTGATCC
AGACCGTGGACTTCTCTCTGGACCCCACCTTCAGCATCGAGATCACCACC
GTTCCTCAGGACGCCGTGTCTCGGTCACAGAGAAGAGGCAGAACAGGCAG
AGGCCGGCTGGGCACATACAGATATGTGACACCCGGCGAAAGACCCAGCG
GCATGTTTGATACAGCCGAGCTGTGCGAGTGTTACGACGCCGGATGTGCT
TGGTACGAGCTGACACCAGCCGAGACAACCACCAGACTGAAGGCCTACTT
CGACACCCCTGGCCTGCCTGTGTGTCAGGACCACCTGGAATTTTGGGAGA
GCGTGTTCACAGGACTGACCCACATCGACGGCCACTTTCTGAGCCAGACC
AAGCAGAGCGGCGAGAACTTCCCTTACCTGGTGGCCTATCAGGCTACCGT
GTCCGCCAAAGTTTGGCTGGCTCCTCCTAGCTGGGACACCATGTGGAAGT
GCCTGATCCGGCTGAAGCCTACACTGCACGGACCTACACCTCTGCTGTAC
AGACTGGGCAGCGTGCAGAATGAGGTGGTGCTGACCCATCCTATCACCAA
GTACATCATGGCCTGCATGAGCGCCGACCTGGAAGTGGTTACA

Hence, preferably the RNA construct is encoded by a DNA sequence substantially as set out in SEQ ID No: 209, or a fragment or variant thereof.

In an embodiment, the RNA sequence corresponding to the codon optimised DNA sequence of SEQ ID No: 209 is provided herein as SEQ ID No: 210, as follows:

[SEQ ID No: 210]
GCCCCUAUCACAGCCUACGCUCAGCAGACCAGAGGCCUGUUCAGCACCAU
CGUGACAAGCCUGACCGGCAGAGACACCAACGAGAAUUGUGGCGAGGUGC
AGGUCCUGUCUACAGCUACCCAGAGCUUUCUGGGCACCGCCGUGAAUGGC
GUGAUGUGGACAGUGUAUCAUGGCGCUGGCGCCAAGACAAUCUCUGGCCC
UAAGGGCCCCGUGAACCAGAUGUACACCAACGUGGACCAGGACCUCGUUG
GCUGGCCUGCUCCUCCUGGUGUUAGAAGCCUGGCUCCUUGUACAUGCGGC
AGCGCCGAUCUGUACCUGGUCACAAGACACGCCGACGUGAUCCCCGUCAG
AAGAAGAGGCGAUACAAGAGGCGCCCUGCUGAGCCCUAGACCUAUCUCUA
UCCUGAAGGGCAGCUCUGGCGGCCCUCUGCUUUGUCCUAUGGGACACAGA
GCCGGCAUCUUCAGAGCCGCCGUGUGUACUAGAGGCGUGGCCAAGGCUGU
GGACUUCGUGCCUGUGGAAAGCCUGGAAACCACCAUGAGAAGCCCCGUGU
UCACCGACAACAGCACCCCUCCAGCUGUGCCUCAGACAUACCAGGUGGCC
CAUCUGCAUGCCCCUACAGGCUCUGGCAAGAGCACAAAAGUGCCUGCCGC
UCAUGCUGCCCAGGGCUAUAAGGUGCUGGUGCUCAAUCCUAGCGUGGCCG
CCACACUCGGCUUUGGCGUGUACAUGUCUAAGGCCUACGGCAUCGACCCC
AACAUCAGAUCUGGCGUGCGGACCAUCACAACAGGCGCCCCAAUCACCUA
CUCUACCUACGGCAAGUUCCUGGCCGAUGGCGGAUGUUCUGGCGGAGCCU
ACGACAUCAUCAUCUGCGACGAGUGCUACAGCACCGACAGCACCACAAUC
CUCGGCAUCGGCACAGUGCUGGAUCAGGCUGAAACAGCCGGCGUCAGACU
GACUGUGCUGGCCACAGCUACACCUCCAGGCAGCGUGACAACCCCUCACA
GCAACAUCGAGGAAGUGGCCCUGCCUACAACCGGCGAGAUCCCAUUCUAU
GGCAAGGCCAUUCCUCUCGAGCUGAUCAAAGGCGGCAGACACCUGAUCUU
UUGCCACAGCAAGAAGAAGUGCGACGAGCUGGCCAGACAGCUGACAUCCC
UGGGACUGAAUGCCGUGGCCUACUACAGAGGACUGGACGUGUCCGUGAUU
CCCACAUCUGGCGACGUGGUCGUGUGUGCCACAGAUGCCCUGAUGACCGG
CUUCACCGGCGACUUCGAUAGCGUGAUCGACUGCAACACCAGCGUGAUCC
AGACCGUGGACUUCUCUCUGGACCCCACCUUCAGCAUCGAGAUCACCACC
GUUCCUCAGGACGCCGUGUCUCGGUCACAGAGAAGAGGCAGAACAGGCAG
AGGCCGGCUGGGCACAUACAGAUAUGUGACACCCGGCGAAAGACCCAGCG
GCAUGUUUGAUACAGCCGAGCUGUGCGAGUGUUACGACGCCGGAUGUGCU
UGGUACGAGCUGACACCAGCCGAGACAACCACCAGACUGAAGGCCUACUU
CGACACCCCUGGCCUGCCUGUGUGUCAGGACCACCUGGAAUUUUGGGAGA
GCGUGUUCACAGGACUGACCCACAUCGACGGCCACUUUCUGAGCCAGACC
AAGCAGAGCGGCGAGAACUUCCCUUACCUGGUGGCCUAUCAGGCUACCGU
GUCCGCCAAAGUUUGGCUGGCUCCUCCUAGCUGGGACACCAUGUGGAAGU
GCCUGAUCCGGCUGAAGCCUACACUGCACGGACCUACACCUCUGCUGUAC
AGACUGGGCAGCGUGCAGAAUGAGGUGGUGCUGACCCAUCCUAUCACCAA
GUACAUCAUGGCCUGCAUGAGCGCCGACCUGGAAGUGGUUACA

Hence, preferably the RNA construct comprises a sequence substantially as set out in SEQ ID No: 210, or a fragment or variant thereof.

In one embodiment, the at least one IIP is DENV NS3 protein (P17763; Genome polyprotein Dengue virus type 1 (strain Nauru/West Pac/1974)), or an orthologue thereof. One embodiment of the polypeptide sequence of DENV NS3 protein is represented herein as SEQ ID No: 211, as follows:

[SEQ ID No: 211]
SGVLWDTPSPPEVERAVLDDGIYRILQRGLLGRSQVGVGVFQEGVFHTMWHVTRGAVLMYQGKRLEPSWASVKKDLIS
YGGGWRFQGSWNAGEEVQVIAVEPGKNPKNVQTAPGTFKTPEGEVGAIALDFKPGTSGSPIVNREGKIVGLYGNGVVT
TSGTYVSAIAQAKASQEGPLPEIEDEVFRKRNLTIMDLHPGSGKTRRYLPAIVREAIRRNVRTLVLAPTRVVASEMAE
ALKGMPIRYQTTAVKSEHTGKEIVDLMCHATFTMRLLSPVRVPNYNMIIMDEAHFTDPASIAARGYISTRVGMGEAAA
IFMTATPPGSVEAFPQSNAVIQDEERDIPERSWNSGYDWITDFPGKTVWFVPSIKSGNDIANCLRKNGKRVVQLSRKT
FDTEYQKTKNNDWDYVVTTDISEMGANFRADRVIDPRRCLKPVILKDGPERVILAGPMPVTVASAAQRRGRIGRNQNK
EGDQYIYMGQPLNNDEDHAHWTEAKMLLDNINTPEGIIPALFEPEREKSAAIDGEYRLRGEARKTFVELMRRGDLPVW
LSYKVASEGFQYSDRRWCFDGERNNQVLEENMDVEIWTKEGERKKLRPRWLDARTYSDPLALREFKEFAAGRR

Therefore, preferably the RNA construct comprises a nucleotide sequence which encodes an amino acid sequence substantially as set out in SEQ ID No: 211, or a variant or fragment thereof.

In one embodiment, the DENV NS3 polypeptide is encoded by the DNA nucleotide sequence of SEQ ID No: 212, as follows:

[SEQ ID No: 212]
TCAGGAGTGCTATGGGACACACCCAGCCCTCCAGAAGTGGAAAGAGCAGTCCTTGATGATGGCATTTATAGAATTCTC
CAAAGAGGATTGTTGGGCAGGTCTCAAGTAGGAGTAGGAGTTTTTCAAGAAGGCGTGTTCCACACAATGTGGCACGTC
ACCAGGGGAGCTGTCCTCATGTACCAAGGGAAGAGACTGGAACCAAGTTGGGCCAGTGTCAAAAAAGACTTGATCTCA
TATGGAGGAGGTTGGAGGTTTCAAGGATCCTGGAACGCGGGAGAAGAAGTGCAGGTGATTGCTGTTGAACCGGGGAAG
AACCCCAAAAATGTACAGACAGCGCCGGGTACCTTCAAGACCCCTGAAGGCGAAGTTGGAGCCATAGCTCTAGACTTT
AAACCCGGCACATCTGGATCTCCTATCGTGAACAGAGAGGGAAAAATAGTAGGTCTTTATGGAAATGGAGTGGTGACA
ACAAGTGGTACCTACGTCAGCGCCATAGCTCAAGCTAAAGCATCACAAGAAGGGCCTCTACCAGAGATTGAGGACGAG
GTGTTTAGGAAAAGAAACTTAACAATAATGGACCTACATCCAGGATCGGGGAAAACAAGAAGATATCTTCCAGCCATA
GTCCGTGAGGCCATAAGAAGGAACGTGCGCACGCTAGTCTTAGCTCCCACAAGAGTTGTCGCTTCTGAAATGGCAGAG
GCGCTCAAGGGAATGCCAATAAGGTATCAGACAACAGCAGTGAAGAGTGAACACACAGGAAAAGAGATAGTTGACCTT
ATGTGTCACGCCACTTTCACTATGCGTCTCCTGTCTCCTGTGAGAGTTCCCAATTATAATATGATTATCATGGATGAA
GCACATTTTACCGATCCAGCCAGCATAGCAGCCAGAGGGTATATCTCAACCCGAGTGGGTATGGGTGAAGCAGCTGCG
ATTTTCATGACAGCCACTCCCCCCGGATCGGTGGAGGCCTTTCCACAGAGCAATGCAGTTATCCAAGATGAGGAAAGA
GACATTCCTGAAAGATCATGGAACTCAGGCTATGACTGGATCACTGATTTCCCAGGTAAAACAGTCTGGTTTGTTCCA
AGCATCAAATCAGGAAATGACATTGCCAACTGTTTAAGAAAGAATGGGAAACGGGTGGTCCAATTGAGCAGAAAAACT
TTTGACACTGAGTACCAGAAAACAAAAAATAACGACTGGGACTATGTTGTCACAACAGACATATCCGAAATGGGAGCA
AACTTCCGAGCCGACAGGGTAATAGACCCGAGGCGGTGCCTGAAACCGGTAATACTAAAAGATGGCCCAGAGCGTGTC
ATTCTAGCCGGACCGATGCCAGTTGACTGTGGCTAGCGCCGCCCAGAGGAGAGGAAGAATTGGAAGGAACCAAAATAAG
GAAGGCGATCAGTATATTTACATGGGACAGCCTCTAAACAATGATGAGGACCACGCCCATTGGACAGAAGCAAAAATG
CTCCTTGACAACATAAACACACCAGAAGGGATTATCCCAGCCCTCTTTGAGCCGGAGAGAGAAAAGAGTGCAGCAATA
GACGGGGAATACAGACTACGGGGTGAAGCGAGGAAAACGTTCGTGGAGCTCATGAGAAGAGGAGATCTACCTGTCTGG
CTATCCTACAAAGTTGCCTCAGAAGGCTTCCAGTACTCCGACAGAAGGTGGTGCTTTGATGGGGAAAGGAACAACCAG
GTGTTGGAGGAGAACATGGACGTGGAGATCTGGACAAAAGAAGGAGAAAGAAAGAAACTACGACCCCGCTGGCTGGAT
GCCAGAACATACTCTGACCCACTGGCTCTGCGCGAATTCAAAGAGTTCGCAGCAGGAAGAAGA

Accordingly, preferably the DENV NS3 polypeptide is encoded by the DNA nucleotide sequence substantially as set out in SEQ ID NO: 212, or a variant or fragment thereof.

One embodiment of codon optimised nucleic acid (DNA) encoding the DENV NS3 polypeptide is provided herein as SEQ ID No: 213, as follows:

[SEQ ID No: 213]
TCTGGCGTGCTGTGGGATACACCTTCTCCACCAGAGGTGGAAAGAGCCGTGCTGGACGACGGCATCTACCGGATTCTG
CAGAGAGGACTGCTGGGCAGATCTCAAGTTGGCGTGGGCGTGTTCCAAGAAGGGGTGTTCCACACCATGTGGCACGTG
ACAAGAGGCGCCGTGCTGATGTACCAGGGCAAGAGACTGGAACCTAGCTGGGCCAGCGTGAAGAAGGACCTGATCTCT
TACGGCGGAGGCTGGCGGTTTCAAGGCTCTTGGAATGCCGGCGAAGAGGTGCAAGTGATCGCCGTGGAACCCGGCAAG
AACCCCAAGAACGTTCAGACAGCCCCTGGCACCTTCAAGACCCCTGAAGGCGAAGTGGGAGCTATCGCCCTGGATTTC
AAGCCTGGCACAAGCGGCAGCCCCATCGTGAACAGAGAAGGCAAGATCGTGGGCCTGTACGGCAATGGCGTGGTCACC
ACATCTGGCACCTACGTGTCAGCCATTGCTCAGGCCAAGGCCTCTCAAGAGGGACCCCTGCCTGAGATCGAGGACGAG
GTGTTCCGGAAGCGGAACCTGACCATCATGGATCTGCACCCTGGCAGCGGCAAGACCAGAAGATATCTGCCCGCCATT
GTGCGCGAGGCCATCCGAAGAAATGTGCGGACACTGGTGCTGGCCCCTACAAGAGTGGTGGCCTCTGAAATGGCCGAG
GCTCTGAAGGGCATGCCTATCAGATACCAGACCACCGCCGTGAAGTCTGAGCACACCGGCAAAGAAATCGTGGACCTG
ATGTGCCACGCCACCTTCACCATGAGACTGCTGAGCCCTGTGCGGGTGCCCAACTACAACATGATCATCATGGACGAG
GCCCACTTCACAGACCCCGCCTCTATTGCCGCCAGAGGCTACATCTCTACCAGAGTCGGCATGGGAGAAGCCGCCGCT
ATCTTCATGACAGCCACACCTCCAGGCAGCGTGGAAGCCTTTCCTCAGTCCAATGCCGTGATCCAGGACGAAGAGAGA
GACATCCCCGAGCGGAGCTGGAACAGCGGCTACGACTGGATCACCGACTTTCCAGGCAAGACCGTTTGGTTCGTGCCC
AGCATCAAGAGCGGCAACGATATCGCCAACTGCCTGCGGAAGAACGGCAAGAGAGTGGTGCAGCTGAGCAGAAAGACC
TTCGACACCGAGTACCAAAAGACCAAGAACAACGACTGGGACTACGTCGTGACCACCGACATCTCTGAGATGGGCGCC
AACTTCAGGGCCGACAGAGTGATCGACCCTCGGAGATGTCTGAAGCCCGTGATCCTGAAGGACGGCCCTGAGAGAGTG
ATTCTGGCCGGACCTATGCCTGTGACAGTGGCTTCTGCCGCTCAGAGAAGAGGCCGGATCGGCCGGAATCAGAACAAA
GAGGGCGACCAGTACATCTACATGGGCCAGCCTCTGAACAACGATGAGGATCACGCCCACTGGACCGAGGCCAAGATG
CTGCTGGACAACATCAACACCCCTGAGGGCATCATCCCCGCTCTGTTCGAGCCCGAGAGAGAGAAGTCTGCCGCAATC
GACGGCGAGTACAGACTGAGAGGCGAGGCCAGAAAGACATTTGTGGAACTGATGCGGAGAGGCGACCTGCCTGTGTGG
CTGAGTTACAAGGTGGCCAGCGAGGGCTTCCAGTACAGCGATAGAAGATGGTGCTTCGATGGCGAGCGGAACAACCAG
GTGCTGGAAGAGAACATGGACGTGGAAATCTGGACCAAAGAAGGCGAGCGCAAGAAACTGCGGCCCAGATGGCTGGAT
GCCCGGACATATTCTGACCCTCTGGCTCTGCGCGAGTTCAAAGAGTTTGCCGCCGGAAGAAGA

Hence, preferably the RNA construct is encoded by a DNA sequence substantially as set out in SEQ ID No: 213, or a fragment or variant thereof.

In an embodiment, the RNA sequence corresponding to the codon optimised DNA sequence of SEQ ID No: 213 is provided herein as SEQ ID No: 214, as follows:

[SEQ ID No: 214]
UCUGGCGUGCUGUGGGAUACACCUUCUCCACCAGAGGUGGAAAGAGCCGUGCUGGACGACGGCAUCUACCGGAUUCUG
CAGAGAGGACUGCUGGGCAGAUCUCAAGUUGGCGUGGGCGUGUUCCAAGAAGGGGUGUUCCACACCAUGUGGCACGUG
ACAAGAGGCGCCGUGCUGAUGUACCAGGGCAAGAGACUGGAACCUAGCUGGGCCAGCGUGAAGAAGGACCUGAUCUCU
UACGGCGGAGGCUGGCGGUUUCAAGGCUCUUGGAAUGCCGGCGAAGAGGUGCAAGUGAUCGCCGUGGAACCCGGCAAG
AACCCCAAGAACGUUCAGACAGCCCCUGGCACCUUCAAGACCCCUGAAGGCGAAGUGGGAGCUAUCGCCCUGGAUUUC
AAGCCUGGCACAAGCGGCAGCCCCAUCGUGAACAGAGAAGGCAAGAUCGUGGGCCUGUACGGCAAUGGCGUGGUCACC
ACAUCUGGCACCUACGUGUCAGCCAUUGCUCAGGCCAAGGCCUCUCAAGAGGGACCCCUGCCUGAGAUCGAGGACGAG
GUGUUCCGGAAGCGGAACCUGACCAUCAUGGAUCUGCACCCUGGCAGCGGCAAGACCAGAAGAUAUCUGCCCGCCAUU
GUGCGCGAGGCCAUCCGAAGAAAUGUGCGGACACUGGUGCUGGCCCCUACAAGAGUGGUGGCCUCUGAAAUGGCCGAG
GCUCUGAAGGGCAUGCCUAUCAGAUACCAGACCACCGCCGUGAAGUCUGAGCACACCGGCAAAGAAAUCGUGGACCUG
AUGUGCCACGCCACCUUCACCAUGAGACUGCUGAGCCCUGUGCGGGUGCCCAACUACAACAUGAUCAUCAUGGACGAG
GCCCACUUCACAGACCCCGCCUCUAUUGCCGCCAGAGGCUACAUCUCUACCAGAGUCGGCAUGGGAGAAGCCGCCGCU
AUCUUCAUGACAGCCACACCUCCAGGCAGCGUGGAAGCCUUUCCUCAGUCCAAUGCCGUGAUCCAGGACGAAGAGAGA
GACAUCCCCGAGCGGAGCUGGAACAGCGGCUACGACUGGAUCACCGACUUUCCAGGCAAGACCGUUUGGUUCGUGCCC
AGCAUCAAGAGCGGCAACGAUAUCGCCAACUGCCUGCGGAAGAACGGCAAGAGAGUGGUGCAGCUGAGCAGAAAGACC
UUCGACACCGAGUACCAAAAGACCAAGAACAACGACUGGGACUACGUCGUGACCACCGACAUCUCUGAGAUGGGCGCC
AACUUCAGGGCCGACAGAGUGAUCGACCCUCGGAGAUGUCUGAAGCCCGUGAUCCUGAAGGACGGCCCUGAGAGAGUG
AUUCUGGCCGGACCUAUGCCUGUGACAGUGGCUUCUGCCGCUCAGAGAAGAGGCCGGAUCGGCCGGAAUCAGAACAAA
GAGGGCGACCAGUACAUCUACAUGGGCCAGCCUCUGAACAACGAUGAGGAUCACGCCCACUGGACCGAGGCCAAGAUG
CUGCUGGACAACAUCAACACCCCUGAGGGCAUCAUCCCCGCUCUGUUCGAGCCCGAGAGAGAGAAGUCUGCCGCAAUC
GACGGCGAGUACAGACUGAGAGGCGAGGCCAGAAAGACAUUUGUGGAACUGAUGCGGAGAGGCGACCUGCCUGUGUGG
CUGAGUUACAAGGUGGCCAGCGAGGGCUUCCAGUACAGCGAUAGAAGAUGGUGCUUCGAUGGCGAGCGGAACAACCAG
GUGCUGGAAGAGAACAUGGACGUGGAAAUCUGGACCAAAGAAGGCGAGCGCAAGAAACUGCGGCCCAGAUGGCUGGAU
GCCCGGACAUAUUCUGACCCUCUGGCUCUGCGCGAGUUCAAAGAGUUUGCCGCCGGAAGAAGA

Hence, preferably the RNA construct comprises a sequence substantially as set out in SEQ ID No: 214, or a fragment or variant thereof.

In one embodiment, the at least one IIP is EV71 3Cpro (B9VUU3; Genome polyprotein Human enterovirus 71), or an orthologue thereof. Lei X, Xiao X, Xue Q, Jin Q, He B, Wang J J. (2013) Cleavage of interferon regulatory factor 7 by enterovirus 71 3C suppresses cellular responses. J Virol, 87, 3, 1690-1698. doi: 10.1128/JVI.01855-12. Epub 2012 Nov. 21.). This IIP is believed to cleave IRF7. One embodiment of the polypeptide sequence of EV71 3Cpro is represented herein as SEQ ID No: 215, as follows:

[SEQ ID No: 215]
GPSLDFALSLLRRNVRQVQTDQGHFTMLGVRDRLAVLPRHSQPGKTIWIEHKLVNVLDAVELVDEQGVNLELTLITLD
TNEKFRDITKFIPENISTASDATLVINTEHMPSMFVPVGDVVQYGFLNLSGKPTHRTMMYNFPTKAGQCGGVVTSVGK
VIGIHIGGNGRQGFCAGLKRSYFASEQ

Therefore, preferably the RNA construct comprises a nucleotide sequence which encodes an amino acid sequence substantially as set out in SEQ ID No: 215, or a variant or fragment thereof.

In one embodiment, the EV71 3Cpro polypeptide is encoded by the DNA nucleotide sequence of SEQ ID No: 216, as follows:

[SEQ ID No: 216]
GGCCCGAGCCTTGATTTTGCTCTCTCCCTACTGAGGAGGAACGTCAGGCAAGTCCAAACAGACCAGGGGCATTTCACC
ATGTTGGGTGTTAGGGATCGCTTAGCAGTCCTCCCACGCCACTCACAACCCGGCAAAACTATTTGGATTGAGCACAAA
CTCGTGAACGTCCTTGATGCAGTTGAATTGGTGGATGAGCAAGGAGTCAACCTGGAATTAACCCTCATCACTCTTGAT
ACCAACGAGAAGTTTAGGGATATCACCAAATTCATCCCGGAAAATATTAGCACTGCTAGTGATGCCACCCTAGTGATC
AACACGGAGCACATGCCCTCGATGTTTGTCCCGGTGGGTGACGTTGTGCAGTATGGTTTCCTGAATCTCAGTGGTAAG
CCTACTCATCGCACCATGATGTACAACTTTCCTACTAAGGCAGGGCAATGTGGAGGGGTGGTGACATCAGTTGGAAAA
GTCATCGGTATACACATAGGTGGCAACGGTAGACAAGGATTTTGTGCAGGTCTTAAGAGAAGCTACTTTGCCAGCGAG
CAA

Accordingly, preferably the EV71 3Cpro polypeptide is encoded by the DNA nucleotide sequence substantially as set out in SEQ ID NO: 216, or a variant or fragment thereof.

One embodiment of codon optimised nucleic acid (DNA) encoding the EV71 3Cpro polypeptide is provided herein as SEQ ID No: 217, as follows:

[SEQ ID No: 217]
GGCCCTTCTCTGGATTTTGCCCTGAGCCTGCTGCGGAGAAATGTGCGCCAGGTGCAGACAGATCAGGGCCACTTTACA
ATGCTGGGCGTCAGAGACAGACTGGCCGTGCTGCCTAGACACTCTCAGCCTGGCAAGACCATCTGGATCGAGCACAAG
CTGGTCAACGTGCTGGACGCCGTGGAACTGGTTGATGAGCAGGGCGTGAACCTGGAACTGACCCTGATCACCCTGGAC
ACCAACGAGAAGTTCCGGGACATCACCAAGTTCATCCCCGAGAACATCAGCACCGCCAGCGACGCCACACTGGTCATC
AATACCGAGCACATGCCCAGCATGTTCGTGCCTGTGGGAGATGTGGTGCAGTACGGCTTCCTGAACCTGAGCGGCAAG
CCCACACACCGGACCATGATGTACAACTTCCCTACCAAGGCCGGCCAGTGCGGCGGAGTGGTTACATCTGTGGGCAAA
GTGATCGGCATCCACATCGGCGGCAATGGCAGACAGGGATTTTGTGCCGGCCTGAAGAGAAGCTACTTCGCCTCTGAA
CAG

Hence, preferably the RNA construct is encoded by a DNA sequence substantially as set out in SEQ ID No: 217, or a fragment or variant thereof.

In an embodiment, the RNA sequence corresponding to the codon optimised DNA sequence of SEQ ID No: 217 is provided herein as SEQ ID No: 218, as follows:

[SEQ ID No: 218]
GGCCCUUCUCUGGAUUUUGCCCUGAGCCUGCUGCGGAGAAAUGUGCGCCAGGUGCAGACAGAUCAGGGCCACUUUACA
AUGCUGGGCGUCAGAGACAGACUGGCCGUGCUGCCUAGACACUCUCAGCCUGGCAAGACCAUCUGGAUCGAGCACAAG
CUGGUCAACGUGCUGGACGCCGUGGAACUGGUUGAUGAGCAGGGCGUGAACCUGGAACUGACCCUGAUCACCCUGGAC
ACCAACGAGAAGUUCCGGGACAUCACCAAGUUCAUCCCCGAGAACAUCAGCACCGCCAGCGACGCCACACUGGUCAUC
AAUACCGAGCACAUGCCCAGCAUGUUCGUGCCUGUGGGAGAUGUGGUGCAGUACGGCUUCCUGAACCUGAGCGGCAAG
CCCACACACCGGACCAUGAUGUACAACUUCCCUACCAAGGCCGGCCAGUGCGGCGGAGUGGUUACAUCUGUGGGCAAA
GUGAUCGGCAUCCACAUCGGCGGCAAUGGCAGACAGGGAUUUUGUGCCGGCCUGAAGAGAAGCUACUUCGCCUCUGAA
CAG

Hence, preferably the RNA construct comprises a sequence substantially as set out in SEQ ID No: 218, or a fragment or variant thereof.

In one embodiment, the at least one IIP is FMDV Lpro (P03307; Leader Protease Genome polyprotein foot-and-mouth disease virus (isolate-/Germany/A5Westerwald/1951 serotype A)), or an orthologue thereof. One embodiment of the polypeptide sequence of FMDV Lpro is represented herein as SEQ ID No: 219, as follows:

[SEQ ID No: 219]
MHTTDCFIALVHAIREIRALFLPRTTGKMELTLHNGEKKTFYSRPNNHDNCWLNTILQLFRYVDEPFFDWVYNSPENL
TLEAINQLEELTGLELHEGGPPALVIWNIKHLLHTGIGTASRPSEVCMVDGTDMCLADFHAGIFLKGQEHAVFACVTS
NGWYAIDDEEFYPWTPDPSDVLVFVPYDQEPLNGDWKAMVQRKLK

Therefore, preferably the RNA construct comprises a nucleotide sequence which encodes an amino acid sequence substantially as set out in SEQ ID No: 219, or a variant or fragment thereof.

In one embodiment, the FMDV Lpro polypeptide is encoded by the DNA nucleotide sequence of SEQ ID No: 220, as follows:

[SEQ ID No: 220]
ATGCATACAACTGACTGTTTTATCGCTTTGGTGCACGCTATCAGAGAGATCAGAGCACTTTTTCTACCACGAACCACA
GGAAAGATGGAACTCACCCTGCACAACGGCGAGAAAAAGACTTTTTACTCTAGACCCAACAACCACGACAACTGCTGG
TTGAACACCATCCTTCAGTTGTTCAGGTATGTCGATGAACCCTTCTTCGACTGGGTCTACAACTCGCCCGAGAACCTC
ACGCTTGAAGCCATCAACCAATTGGAGGAACTCACAGGACTTGAGTTGCACGAGGGCGGACCGCCTGCCCTTGTGATC
TGGAACATCAAACACTTGCTCCACACCGGCATCGGCACCGCCTCACGACCCAGTGAGGTGTGTATGGTGGACGGCACG
GACATGTGTCTTGCTGACTTCCACGCAGGCATTTTCCTGAAGGGACAGGAACACGCAGTCTTTGCGTGTGTCACCTCC
AACGGGTGGTACGCGATTGACGACGAGGAATTTTACCCCTGGACGCCTGACCCGTCAGACGTCCTGGTGTTTGTCCCG
TACGATCAAGAACCACTCAACGGGGACTGGAAAGCGATGGTTCAGAGGAAGCTTAAG

Accordingly, preferably the FMDV Lpro polypeptide is encoded by the DNA nucleotide sequence substantially as set out in SEQ ID NO: 220, or a variant or fragment thereof.

One embodiment of codon optimised nucleic acid (DNA) encoding the FMDV Lpro polypeptide is provided herein as SEQ ID No: 221, as follows:

[SEQ ID No: 221]
ATGCACACCACCGACTGCTTTATCGCCCTGGTGCACGCCATCAGAGAGATCAGAGCCCTGTTCCTGCCTCGGACCACC
GGCAAGATGGAACTGACACTGCACAACGGCGAGAAGAAAACCTTCTACAGCAGACCCAACAACCACGACAACTGCTGG
CTGAACACCATCCTGCAGCTGTTCAGATACGTGGACGAGCCCTTCTTCGACTGGGTGTACAACAGCCCCGAGAATCTG
ACCCTGGAAGCCATCAACCAGCTGGAAGAACTGACCGGCCTGGAACTGCATGAAGGCGGACCTCCAGCTCTGGTCATC
TGGAACATCAAACATCTGCTGCACACCGGCATCGGCACCGCCTCTAGACCATCTGAAGTGTGCATGGTGGACGGCACC
GATATGTGCCTGGCCGATTTTCACGCCGGCATCTTTCTGAAGGGCCAAGAGCATGCCGTGTTCGCCTGCGTGACAAGC
AATGGATGGTACGCCATCGACGACGAGGAATTCTACCCCTGGACACCCGATCCTAGCGACGTGCTGGTGTTCGTGCCC
TACGATCAAGAGCCCCTGAACGGCGATTGGAAGGCCATGGTGCAGCGGAAGCTGAAG

Hence, preferably the RNA construct is encoded by a DNA sequence substantially as set out in SEQ ID No: 221, or a fragment or variant thereof.

In an embodiment, the RNA sequence corresponding to the codon optimised DNA sequence of SEQ ID No: 221 is provided herein as SEQ ID No: 222, as follows:

[SEQ ID No: 222]
AUGCACACCACCGACUGCUUUAUCGCCCUGGUGCACGCCAUCAGAGAGAUCAGAGCCCUGUUCCUGCCUCGGACCACC
GGCAAGAUGGAACUGACACUGCACAACGGCGAGAAGAAAACCUUCUACAGCAGACCCAACAACCACGACAACUGCUGG
CUGAACACCAUCCUGCAGCUGUUCAGAUACGUGGACGAGCCCUUCUUCGACUGGGUGUACAACAGCCCCGAGAAUCUG
ACCCUGGAAGCCAUCAACCAGCUGGAAGAACUGACCGGCCUGGAACUGCAUGAAGGCGGACCUCCAGCUCUGGUCAUC
UGGAACAUCAAACAUCUGCUGCACACCGGCAUCGGCACCGCCUCUAGACCAUCUGAAGUGUGCAUGGUGGACGGCACC
GAUAUGUGCCUGGCCGAUUUUCACGCCGGCAUCUUUCUGAAGGGCCAAGAGCAUGCCGUGUUCGCCUGCGUGACAAGC
AAUGGAUGGUACGCCAUCGACGACGAGGAAUUCUACCCCUGGACACCCGAUCCUAGCGACGUGCUGGUGUUCGUGCCC
UACGAUCAAGAGCCCCUGAACGGCGAUUGGAAGGCCAUGGUGCAGCGGAAGCUGAAG

Hence, preferably the RNA construct comprises a sequence substantially as set out in SEQ ID No: 222, or a fragment or variant thereof.

In one embodiment, the at least one IIP is FMDV 3Cpro (P03307; Protease 3C Genome polyprotein foot-and-mouth disease virus (isolate-/Germany/A5Westerwald/1951 serotype A), or an orthologue thereof. Ekanayaka P, Shin S H, Weeratunga P, Lee H, Kim T-H, Chathuranga K, Subasinghe A, Park J-H, Lee J-S (2021) Foot and mouth disease virus 3C protease antagonises interferon signaling and C142T substitution attenuates the FMD virus. Front Microbiol., 21, 737031. doi: 10.3389/fmicb.2021.737031

One embodiment of the polypeptide sequence of FMDV 3Cpro is represented herein as SEQ ID No: 223, as follows:

[SEQ ID No: 223]
SGAPPTDLQKMVMGNTKPVELILDGKTVAICCATGVFGTAYLVPRHLFAEKYDKIMLDGRAMTDSDYRVFEFEIKVKG
QDMLSDAALMVLHRGNRVRDITKHFRDTARMKKGTPVVGVINNADVGRLIFSGEALTYKDIVVCMDGDTMPGLFAYRA
ATKAGYCGGAVLAKDGADTFIVGTHSAGGNGVGYCSCVSRSMLLKMKAHIDPEPHHE

Therefore, preferably the RNA construct comprises a nucleotide sequence which encodes an amino acid sequence substantially as set out in SEQ ID No: 223, or a variant or fragment thereof.

In one embodiment, the FMDV 3Cpro polypeptide is encoded by the DNA nucleotide sequence of SEQ ID No: 224, as follows:

[SEQ ID No: 224]
AGTGGTGCCCCCCCGACCGACTTGCAAAAGATGGTCATGGGCAACACAAAGCCTGTTGAGCTCATCCTCGACGGGAAG
ACAGTAGCCATCTGCTGTGCTACTGGAGTGTTTGGCACTGCCTACCTCGTGCCTCGTCATCTTTTCGCTGAGAAGTAT
GACAAGATCATGTTGGACGGCAGAGCCATGACAGACAGTGACTACAGAGTGTTTGAGTTCGAGATCAAAGTAAAAGGA
CAGGACATGCTCTCAGACGCCGCACTCATGGTGCTCCACCGTGGGAACCGCGTGAGAGACATCACGAAGCACTTTCGT
GACACAGCAAGAATGAAGAAAGGCACCCCCGTTGTCGGCGTGATCAACAATGCCGATGTCGGGAGACTGATTTTCTCT
GGCGAAGCCCTTACCTACAAAGACATTGTAGTGTGCATGGACGGAGACACCATGCCCGGGCTTTTTGCCTACAGAGCC
GCCACTAAGGCAGGCTACTGCGGGGGAGCCGTTCTCGCTAAGGACGGGGCTGACACTTTCATCGTTGGCACTCACTCT
GCAGGAGGTAATGGAGTTGGATACTGCTCATGCGTTTCCAGGTCCATGCTTCTCAAGATGAAGGCACACATTGACCCT
GAGCCGCACCACGAG

Accordingly, preferably the FMDV 3Cpro polypeptide is encoded by the DNA nucleotide sequence substantially as set out in SEQ ID NO: 224, or a variant or fragment thereof.

One embodiment of codon optimised nucleic acid (DNA) encoding the FMDV 3Cpro polypeptide is provided herein as SEQ ID No: 225, as follows:

[SEQ ID No: 225]
TCTGGTGCCCCTCCTACCGACCTGCAGAAAATGGTCATGGGCAACACCAAGCCTGTGGAACTGATCCTGGACGGCAAG
ACCGTGGCCATCTGTTGTGCAACAGGCGTGTTCGGCACCGCCTACCTGGTTCCTAGACACCTGTTCGCCGAGAAGTAC
GACAAGATCATGCTGGATGGCAGAGCCATGACCGACAGCGACTACCGGGTGTTCGAGTTCGAGATCAAAGTGAAAGGC
CAGGACATGCTGAGCGACGCCGCTCTGATGGTTCTGCACAGAGGCAACAGAGTGCGGGACATCACCAAGCACTTCCGG
GACACCGCCAGAATGAAGAAAGGCACACCTGTCGTGGGCGTGATCAACAACGCTGACGTGGGCAGACTGATCTTCTCT
GGCGAGGCCCTGACCTACAAGGACATCGTCGTGTGCATGGACGGCGACACAATGCCTGGCCTGTTTGCCTATAGAGCC
GCCACAAAGGCCGGCTACTGTGGCGGAGCTGTGCTGGCTAAAGATGGCGCCGATACCTTCATCGTGGGCACACATTCT
GCCGGCGGAAATGGCGTGGGCTACTGCTCTTGTGTGTCCAGATCCATGCTGCTGAAGATGAAGGCCCACATCGACCCC
GAGCCTCACCATGAA

Hence, preferably the RNA construct is encoded by a DNA sequence substantially as set out in SEQ ID No: 225, or a fragment or variant thereof.

In an embodiment, the RNA sequence corresponding to the codon optimised DNA sequence of SEQ ID No: 225 is provided herein as SEQ ID No: 226, as follows:

[SEQ ID No: 226]
UCUGGUGCCCCUCCUACCGACCUGCAGAAAAUGGUCAUGGGCAACACCAAGCCUGUGGAACUGAUCCUGGACGGCAAG
ACCGUGGCCAUCUGUUGUGCAACAGGCGUGUUCGGCACCGCCUACCUGGUUCCUAGACACCUGUUCGCCGAGAAGUAC
GACAAGAUCAUGCUGGAUGGCAGAGCCAUGACCGACAGCGACUACCGGGUGUUCGAGUUCGAGAUCAAAGUGAAAGGC
CAGGACAUGCUGAGCGACGCCGCUCUGAUGGUUCUGCACAGAGGCAACAGAGUGCGGGACAUCACCAAGCACUUCCGG
GACACCGCCAGAAUGAAGAAAGGCACACCUGUCGUGGGCGUGAUCAACAACGCUGACGUGGGCAGACUGAUCUUCUCU
GGCGAGGCCCUGACCUACAAGGACAUCGUCGUGUGCAUGGACGGCGACACAAUGCCUGGCCUGUUUGCCUAUAGAGCC
GCCACAAAGGCCGGCUACUGUGGCGGAGCUGUGCUGGCUAAAGAUGGCGCCGAUACCUUCAUCGUGGGCACACAUUCU
GCCGGCGGAAAUGGCGUGGGCUACUGCUCUUGUGUGUCCAGAUCCAUGCUGCUGAAGAUGAAGGCCCACAUCGACCCC
GAGCCUCACCAUGAA

Hence, preferably the RNA construct comprises a sequence substantially as set out in SEQ ID No: 226, or a fragment or variant thereof.

In one embodiment, the at least one IIP is Toscana Virus NSS protein (RIG 1 degradation) (P21699; Non-structural protein NS-S Toscana virus), or an orthologue thereof. One embodiment of the polypeptide sequence of Toscana Virus NSS protein is represented herein as SEQ ID No: 227, as follows:

[SEQ ID No: 227]
MQSRAVILKYRSGSGHKRSLPRFYIDCDLDTFDFEKDCSLIENEFPIYINNYKVVYKSKPTLSHFLIEKEFPAVLGPG
MISAVRTRLYEPTMRELYQESIHQLKRSNKKYLLSALRWPTGIPTLEFTDYYFEELLFLSEFDPGSIQRYLKLLVKAS
GLYNSTNEEQIVEIHRRVLIEGKKHGLTAFDLPGNDILGDICVVQAARVTRLVAKTFSKMTRDTHLMIYFSISPVELV
LSKLDKKGDKRAKAKGLMSMSAARSYDYFMRTDLGFRETALSTFWAKDWPTPQETILSDKRCLKEDMRVTKWLPSPPH
YPPL

Therefore, preferably the RNA construct comprises a nucleotide sequence which encodes an amino acid sequence substantially as set out in SEQ ID No: 227, or a variant or fragment thereof.

In one embodiment, the Toscana Virus NSS polypeptide is encoded by the DNA nucleotide sequence of SEQ ID No: 228, as follows:

[SEQ ID No: 228]
ATGCAATCCAGAGCTGTCATCTTGAAGTATAGATCTGGTTCAGGCCACAAGAGGTCTTTGCCCAGGTTCTACATAGAC
TGTGATTTGGACACCTTTGATTTTGAGAAGGATTGCTCTCTGATTGAGAATGAGTTCCCCATTTACATAAACAATTAT
AAGGTGGTCTATAAGTCAAAGCCAACTCTCTCACATTTCCTCATTGAGAAGGAGTTTCCTGCTGTGCTGGGGCCTGGT
ATGATCAGTGCAGTTCGAACCAGACTTTACGAGCCAACTATGAGAGAGCTCTACCAGGAATCGATTCACCAACTAAAG
AGGAGCAACAAGAAATACCTTTTGTCTGCTCTCAGGTGGCCCACAGGGATTCCTACTCTAGAGTTTATAGACTATTAC
TTCGAGGAGCTCCTGTTCTTGTCAGAGTTTGACCCGGGGTCTATCCAGAGATACCTGAAATTACTGGTTAAGGCCTCT
GGGCTTTACAACTCCACTAATGAGGAGCAGATAGTGGAGATTCACAGACGAGTGCTCATAGAAGGCAAAAAGCACGGA
TTGACTGCTTTTGATCTCCCAGGAAATGACATCCTTGGAGACATCTGTGTGGTCCAAGCAGCACGGGTGACAAGACTG
GTTGCTAAGACATTCTCTAAGATGACCAGAGACACCCATCTGATGATATACTTCTCGATAAGCCCAGTTGAGTTGGTT
TTGAGTAAACTTGATAAGAAAGGGGACAAGAGGGCTAAAGCAAAAGGGTTGATGTCTATGAGTGCCGCTAGGTCTTAT
GACTATTTTATGAGAACTGACTTGGGATTCAGAGAGACTGCTCTTTCCACCTTTTGGGCTAAGGACTGGCCTACCCCA
CAAGAGACCATTCTATCTGACAAACGATGCCTTAAAGAAGACATGAGAGTGACAAAGTGGCTGCCTAGTCCCCCCCAC
TACCCACCCTTA

Accordingly, preferably the Toscana Virus NSS polypeptide is encoded by the DNA nucleotide sequence substantially as set out in SEQ ID NO: 228, or a variant or fragment thereof.

One embodiment of codon optimised nucleic acid (DNA) encoding the Toscana Virus NSS polypeptide is provided herein as SEQ ID No: 229, as follows:

[SEQ ID No: 229]
ATGCAGAGCAGAGCCGTGATCCTGAAGTACAGAAGCGGCAGCGGCCACAAGAGAAGCCTGCCTAGATTCTACATCGAC
TGCGACCTGGACACCTTCGACTTCGAGAAGGACTGCAGCCTGATCGAGAACGAGTTCCCCATCTACATCAACAACTAC
AAGGTGGTGTACAAGAGCAAGCCAACTCTGAGCCACTTCCTCATCGAGAAAGAATTCCCTGCCGTGCTCGGCCCTGGC
ATGATCTCTGCCGTTAGAACCAGACTGTACGAGCCCACCATGAGAGAGCTGTACCAAGAGAGCATCCACCAGCTGAAG
CGGAGCAACAAGAAGTACCTGCTGAGCGCCCTGAGATGGCCCACAGGCATTCCCACACTGGAATTCATCGACTACTAC
TTCGAGGAACTGCTGTTCCTGAGCGAGTTCGACCCTGGCAGCATCCAGAGATACCTGAAGCTGCTGGTCAAGGCCAGC
GGCCTGTACAACAGCACCAACGAGGAACAGATCGTGGAAATCCACCGGCGGGTGCTGATCGAGGGAAAGAAGCACGGA
CTGACCGCCTTCGACCTGCCTGGCAATGATATCCTGGGCGACATCTGCGTGGTGCAGGCCGCTAGAGTGACAAGACTG
GTGGCCAAGACCTTCAGCAAGATGACCAGAGACACCCACCTGATGATCTACTTCAGCATCAGCCCCGTGGAACTGGTG
CTGAGCAAGCTGGACAAGAAGGGCGACAAGAGAGCCAAGGCCAAGGGCCTGATGAGCATGTCTGCCGCCAGATCCTAC
GACTACTTCATGAGAACCGACCTGGGCTTCAGAGAGACAGCCCTGAGCACCTTCTGGGCCAAAGACTGGCCCACACCT
CAAGAGACAATCCTGTCCGACAAGCGGTGCCTGAAAGAAGATATGCGGGTCACCAAGTGGCTGCCCTCTCCACCTCAT
TACCCTCCACTT

Hence, preferably the RNA construct is encoded by a DNA sequence substantially as set out in SEQ ID No: 229, or a fragment or variant thereof.

In an embodiment, the RNA sequence corresponding to the codon optimised DNA sequence of SEQ ID No: 229 is provided herein as SEQ ID No: 230, as follows:

[SEQ ID No: 230]
AUGCAGAGCAGAGCCGUGAUCCUGAAGUACAGAAGCGGCAGCGGCCACAAGAGAAGCCUGCCUAGAUUCUACAUCGAC
UGCGACCUGGACACCUUCGACUUCGAGAAGGACUGCAGCCUGAUCGAGAACGAGUUCCCCAUCUACAUCAACAACUAC
AAGGUGGUGUACAAGAGCAAGCCAACUCUGAGCCACUUCCUCAUCGAGAAAGAAUUCCCUGCCGUGCUCGGCCCUGGC
AUGAUCUCUGCCGUUAGAACCAGACUGUACGAGCCCACCAUGAGAGAGCUGUACCAAGAGAGCAUCCACCAGCUGAAG
CGGAGCAACAAGAAGUACCUGCUGAGCGCCCUGAGAUGGCCCACAGGCAUUCCCACACUGGAAUUCAUCGACUACUAC
UUCGAGGAACUGCUGUUCCUGAGCGAGUUCGACCCUGGCAGCAUCCAGAGAUACCUGAAGCUGCUGGUCAAGGCCAGC
GGCCUGUACAACAGCACCAACGAGGAACAGAUCGUGGAAAUCCACCGGCGGGUGCUGAUCGAGGGAAAGAAGCACGGA
CUGACCGCCUUCGACCUGCCUGGCAAUGAUAUCCUGGGCGACAUCUGCGUGGUGCAGGCCGCUAGAGUGACAAGACUG
GUGGCCAAGACCUUCAGCAAGAUGACCAGAGACACCCACCUGAUGAUCUACUUCAGCAUCAGCCCCGUGGAACUGGUG
CUGAGCAAGCUGGACAAGAAGGGCGACAAGAGAGCCAAGGCCAAGGGCCUGAUGAGCAUGUCUGCCGCCAGAUCCUAC
GACUACUUCAUGAGAACCGACCUGGGCUUCAGAGAGACAGCCCUGAGCACCUUCUGGGCCAAAGACUGGCCCACACCU
CAAGAGACAAUCCUGUCCGACAAGCGGUGCCUGAAAGAAGAUAUGCGGGUCACCAAGUGGCUGCCCUCUCCACCUCAU
UACCCUCCACUU

Hence, preferably the RNA construct comprises a sequence substantially as set out in SEQ ID No: 230, or a fragment or variant thereof.

In one embodiment, the at least one IIP is Non-structural protein 1 Influenza A virus (strain A/Puerto Rico/8/1934 H1N1) (P03496; Influenza A/PR/8/34 NS1), or an orthologue thereof. One embodiment of the polypeptide sequence of Influenza A virus Non-structural protein 1 is represented herein as SEQ ID No: 231, as follows:

[SEQ ID No: 231]
MDPNTVSSFQVDCFLWHVRKRVADQELGDAPFLDRLRRDQKSLRGRGSTLGLDIETATRAGKQIVERILKEESDEALK
MTMASVPASRYLTDMTLEEMSREWSMLIPKQKVAGPLCIRMDQAIMDKNIILKANFSVIFDRLETLILLRAFTEEGAI
VGEISPLPSLPGHTAEDVKNAVGVLIGGLEWNDNTVRVSETLQRFAWRSSNENGRPPLTPKQKREMAGTIRSEV

Therefore, preferably the RNA construct comprises a nucleotide sequence which encodes an amino acid sequence substantially as set out in SEQ ID No: 231, or a variant or fragment thereof.

In one embodiment, the Non-structural protein 1 Influenza A virus polypeptide is encoded by the DNA nucleotide sequence of SEQ ID No: 232, as follows:

[SEQ ID No: 232]
ATGGATCCAAACACTGTGTCAAGCTTTCAGGTAGATTGCTTTCTTTGGCATGTCCGCAAACGAGTTGCAGACCAAGAA
CTAGGTGATGCCCCATTCCTTGATCGGCTTCGCCGAGATCAGAAATCCCTAAGAGGAAGGGGCAGCACTCTTGGTCTG
GACATCGAGACAGCCACACGTGCTGGAAAGCAGATAGTGGAGCGGATTCTGAAAGAAGAATCCGATGAGGCACTTAAA
ATGACCATGGCCTCTGTACCTGCGTCGCGTTACCTAACCGACATGACTCTTGAGGAAATGTCAAGGGAATGGTCCATG
CTCATACCCAAGCAGAAAGTGGCAGGCCCTCTTTGTATCAGAATGGACCAGGCGATCATGGATAAAAACATCATACTG
AAAGCGAACTTCAGTGTGATTTTTGACCGGCTGGAGACTCTAATATTGCTAAGGGCTTTCACCGAAGAGGGAGCAATT
GTTGGCGAAATTTCACCATTGCCTTCTCTTCCAGGACATACTGCTGAGGATGTCAAAAATGCAGTTGGAGTCCTCATC
GGAGGACTTGAATGGAATGATAACACAGTTCGAGTCTCTGAAACTCTACAGAGATTCGCTTGGAGAAGCAGTAATGAG
AATGGGAGACCTCCACTCACTCCAAAACAGAAACGAGAAATGGCGGGAACAATTAGGTCAGAAGTTTGA

Accordingly, preferably the Non-structural protein 1 Influenza A virus polypeptide is encoded by the DNA nucleotide sequence substantially as set out in SEQ ID NO: 232, or a variant or fragment thereof.

One embodiment of codon optimised nucleic acid (DNA) encoding the Non-structural protein 1 Influenza A virus polypeptide is provided herein as SEQ ID No: 233, as follows:

[SEQ ID No: 233]
ATGGACCCCAATACCGTCAGCAGCTTCCAGGTGGACTGCTTCCTGTGGCACGTGCGGAAAAGAGTGGCCGATCAAGAA
CTGGGCGACGCCCCATTCCTGGACAGACTGAGAAGAGATCAGAAGTCCCTGAGAGGCAGAGGCAGCACACTGGGCCTC
GACATTGAGACAGCCACAAGAGCCGGCAAGCAGATCGTGGAACGGATCCTGAAAGAGGAAAGCGACGAGGCCCTGAAG
ATGACCATGGCCTCTGTGCCTGCCAGCAGATACCTGACCGACATGACCCTGGAAGAGATGAGCCGCGAGTGGTCCATG
CTGATCCCCAAGCAGAAAGTGGCCGGACCTCTGTGCATCAGAATGGATCAGGCCATCATGGACAAGAACATCATCCTG
AAGGCCAACTTCAGCGTGATCTTCGACCGGCTGGAAACCCTGATCCTGCTGAGAGCCTTTACCGAAGAGGGCGCCATC
GTGGGAGAGATCAGTCCTCTGCCTTCTCTGCCTGGACACACCGCCGAGGATGTGAAGAATGCTGTGGGCGTGCTGATC
GGCGGCCTGGAATGGAACGATAACACCGTCAGAGTGTCCGAGACACTGCAGAGATTTGCCTGGCGGAGCAGCAACGAG
AACGGCAGACCTCCTCTGACACCTAAGCAGAAAAGAGAGATGGCCGGCACCATCCGCAGCGAAGTGTAA

Hence, preferably the RNA construct is encoded by a DNA sequence substantially as set out in SEQ ID No: 233, or a fragment or variant thereof.

In an embodiment, the RNA sequence corresponding to the codon optimised DNA sequence of SEQ ID No: 233 is provided herein as SEQ ID No: 234, as follows:

[SEQ ID No: 234]
AUGGACCCCAAUACCGUCAGCAGCUUCCAGGUGGACUGCUUCCUGUGGCACGUGCGGAAAAGAGUGGCCGAUCAAGAA
CUGGGCGACGCCCCAUUCCUGGACAGACUGAGAAGAGAUCAGAAGUCCCUGAGAGGCAGAGGCAGCACACUGGGCCUC
GACAUUGAGACAGCCACAAGAGCCGGCAAGCAGAUCGUGGAACGGAUCCUGAAAGAGGAAAGCGACGAGGCCCUGAAG
AUGACCAUGGCCUCUGUGCCUGCCAGCAGAUACCUGACCGACAUGACCCUGGAAGAGAUGAGCCGCGAGUGGUCCAUG
CUGAUCCCCAAGCAGAAAGUGGCCGGACCUCUGUGCAUCAGAAUGGAUCAGGCCAUCAUGGACAAGAACAUCAUCCUG
AAGGCCAACUUCAGCGUGAUCUUCGACCGGCUGGAAACCCUGAUCCUGCUGAGAGCCUUUACCGAAGAGGGCGCCAUC
GUGGGAGAGAUCAGUCCUCUGCCUUCUCUGCCUGGACACACCGCCGAGGAUGUGAAGAAUGCUGUGGGCGUGCUGAUC
GGCGGCCUGGAAUGGAACGAUAACACCGUCAGAGUGUCCGAGACACUGCAGAGAUUUGCCUGGCGGAGCAGCAACGAG
AACGGCAGACCUCCUCUGACACCUAAGCAGAAAAGAGAGAUGGCCGGCACCAUCCGCAGCGAAGUGUAA

Hence, preferably the RNA construct comprises a sequence substantially as set out in SEQ ID No: 234, or a fragment or variant thereof.

In one embodiment, the at least one IIP is SARS CoV N protein (P59595; Nucleoprotein Severe acute respiratory syndrome coronavirus), or an orthologue thereof. One embodiment of the polypeptide sequence of SARS CoV N protein is represented herein as SEQ ID No: 235, as follows:

[SEQ ID No: 235]
MSDNGPQSNQRSAPRITFGGPTDSTDNNQNGGRNGARPKQRRPQGLPNNTASWFTALTQHGKEELRFPRGQGVPINTN
SGPDDQIGYYRRATRRVRGGDGKMKELSPRWYFYYLGTGPEASLPYGANKEGIVWVATEGALNTPKDHIGTRNPNNNA
ATVLOLPQGTTLPKGFYAEGSRGGSQASSRSSSRSRGNSRNSTPGSSRGNSPARMASGGGETALALLLLDRLNQLESK
VSGKGQQQQGQTVTKKSAAEASKKPRQKRTATKQYNVTQAFGRRGPEQTQGNFGDQDLIRQGTDYKHWPQIAQFAPSA
SAFFGMSRIGMEVTPSGTWLTYHGAIKLDDKDPQFKDNVILLNKHIDAYKTFPPTEPKKDKKKKTDEAQPLPQRQKKQ
PTVTLLPAADMDDFSRQLQNSMSGASADSTQA

Therefore, preferably the RNA construct comprises a nucleotide sequence which encodes an amino acid sequence substantially as set out in SEQ ID No: 235, or a variant or fragment thereof.

In one embodiment, the SARS CoV N polypeptide is encoded by the DNA nucleotide sequence of SEQ ID No: 236, as follows:

[SEQ ID No: 236]
ATGTCTGATAATGGACCCCAATCAAACCAACGTAGTGCCCCCCGCATTACATTTGGTGGACCCACAGATTCAACTGAC
AATAACCAGAATGGAGGACGCAATGGGGCAAGGCCAAAACAGCGCCGACCCCAAGGTTTACCCAATAATACTGCGTCT
TGGTTCACAGCTCTCACTCAGCATGGCAAGGAGGAACTTAGATTCCCTCGAGGCCAGGGCGTTCCAATCAACACCAAT
AGTGGTCCAGATGACCAAATTGGCTACTACCGAAGAGCTACCCGACGAGTTCGTGGTGGTGACGGCAAAATGAAAGAG
ATCGTATGGGTTGCAACTGAGGGAGCCTTGAATACACCCAAAGACCACATTGGCACCCGCAATCCTAATAACAATGCT
GCCACCGTGCTACAACTTCCTCAAGGAACAACATTGCCAAAAGGCTTCTACGCAGAGGGAAGCAGAGGCGGCAGTCAA
GCCTCTTCTCGCTCCTCATCACGTAGTCGCGGTAATTCAAGAAATTCAACTCCTGGCAGCAGTAGGGGAAATTCTCCT
GCTCGAATGGCTAGCGGAGGTGGTGAAACTGCCCTCGCGCTATTGCTGCTAGACAGATTGAACCAGCTTGAGAGCAAA
GTTTCTGGTAAAGGCCAACAACAACAAGGCCAAACTGTCACTAAGAAATCTGCTGCTGAGGCATCTAAAAAGCCTCGC
CAAAAACGTACTGCCACAAAACAGTACAACGTCACTCAAGCATTTGGGAGACGTGGTCCAGAACAAACCCAAGGAAAT
TTCGGGGACCAAGACCTAATCAGACAAGGAACTGATTACAAACATTGGCCGCAAATTGCACAATTTGCTCCAAGTGCC
TCTGCATTCTTTGGAATGTCACGCATTGGCATGGAAGTCACACCTTCGGGAACATGGCTGACTTATCATGGAGCCATT
AAATTGGATGACAAAGATCCACAATTCAAAGACAACGTCATACTGCTGAACAAGCACATTGACGCATACAAAACATTC
CCACCAACAGAGCCTAAAAAGGACAAAAAGAAAAAGACTGATGAAGCTCAGCCTTTGCCGCAGAGACAAAAGAAGCAG
CCCACTGTGACTCTTCTTCCTGCGGCTGACATGGATGATTTCTCCAGACAACTTCAAAATTCCATGAGTGGAGCTTCT
GCTGATTCAACTCAGGCA

Accordingly, preferably the SARS CoV N polypeptide is encoded by the DNA nucleotide sequence substantially as set out in SEQ ID NO: 236, or a variant or fragment thereof.

One embodiment of codon optimised nucleic acid (DNA) encoding the SARS CoV N polypeptide is provided herein as SEQ ID No: 237, as follows:

[SEQ ID No: 237]
ATGAGCGACAATGGCCCTCAGAGCAACCAGAGAAGCGCCCCTAGAATCACCTTTGGCGGCCCTACCGACAGCACCGAC
AACAACCAGAACGGCGGCAGAAATGGCGCCAGACCTAAGCAGAGAAGGCCTCAGGGCCTGCCTAACAATACCGCCAGC
TGGTTCACAGCCCTGACACAGCACGGCAAAGAGGAACTGAGATTCCCCAGAGGACAGGGCGTGCCCATCAACACAAAT
AGCGGCCCTGACGACCAGATCGGCTACTACAGACGGGCCACCAGAAGAGTTAGAGGCGGCGACGGCAAGATGAAGGAA
CTGTCCCCTCGGTGGTACTTCTACTACCTCGGCACAGGACCCGAAGCCAGCCTTCCTTATGGCGCCAACAAAGAGGGC
ATCGTCTGGGTTGCAACAGAAGGCGCCCTGAACACCCCTAAGGACCACATCGGCACCAGAAATCCCAACAACAACGCC
GCCACAGTGCTGCAGTTGCCACAGGGAACAACACTGCCCAAGGGCTTCTACGCCGAGGGATCTAGAGGCGGATCTCAG
GCCAGCAGCAGAAGCAGCTCTAGAAGCAGAGGCAACAGCCGGAATAGCACCCCTGGCAGCTCCAGAGGCAATTCCCCT
GCCAGAATGGCTTCTGGCGGCGGAGAAACAGCTCTGGCACTGCTGCTGCTCGACCGGCTGAATCAGCTGGAATCTAAG
GTGTCCGGCAAGGGCCAGCAACAGCAGGGACAGACCGTGACCAAGAAGTCTGCCGCTGAGGCCAGCAAGAAGCCCAGA
CAGAAGAGAACCGCCACCAAGCAGTACAACGTGACCCAGGCCTTTGGCAGAAGAGGCCCAGAACAGACCCAGGGCAAT
TTCGGCGACCAGGACCTGATCAGACAGGGCACCGATTACAAGCACTGGCCCCAGATCGCCCAGTTTGCCCCTTCTGCC
TCTGCCTTTTTCGGCATGAGCCGGATCGGCATGGAAGTGACACCTAGCGGCACCTGGCTGACATATCACGGCGCCATC
AAGCTGGACGACAAGGACCCTCAGTTCAAGGACAACGTGATCCTGCTGAACAAGCACATCGACGCCTACAAGACATTC
CCTCCAACCGAGCCTAAGAAGGACAAGAAGAAGAAAACCGACGAGGCCCAGCCTCTGCCACAGAGACAGAAAAAGCAG
CCCACCGTGACACTGCTGCCTGCCGCCGATATGGACGACTTCTCTAGACAGCTGCAGAACAGCATGAGCGGCGCCAGC
GCTGATTCTACACAAGCT

Hence, preferably the RNA construct is encoded by a DNA sequence substantially as set out in SEQ ID No: 237, or a fragment or variant thereof.

In an embodiment, the RNA sequence corresponding to the codon optimised DNA sequence of SEQ ID No: 237 is provided herein as SEQ ID No: 238, as follows:

[SEQ ID No: 238]
AUGAGCGACAAUGGCCCUCAGAGCAACCAGAGAAGCGCCCCUAGAAUCACCUUUGGCGGCCCUACCGACAGCACCGAC
AACAACCAGAACGGCGGCAGAAAUGGCGCCAGACCUAAGCAGAGAAGGCCUCAGGGCCUGCCUAACAAUACCGCCAGC
UGGUUCACAGCCCUGACACAGCACGGCAAAGAGGAACUGAGAUUCCCCAGAGGACAGGGCGUGCCCAUCAACACAAAU
AGCGGCCCUGACGACCAGAUCGGCUACUACAGACGGGCCACCAGAAGAGUUAGAGGCGGCGACGGCAAGAUGAAGGAA
CUGUCCCCUCGGUGGUACUUCUACUACCUCGGCACAGGACCCGAAGCCAGCCUUCCUUAUGGCGCCAACAAAGAGGGC
AUCGUCUGGGUUGCAACAGAAGGCGCCCUGAACACCCCUAAGGACCACAUCGGCACCAGAAAUCCCAACAACAACGCC
GCCACAGUGCUGCAGUUGCCACAGGGAACAACACUGCCCAAGGGCUUCUACGCCGAGGGAUCUAGAGGCGGAUCUCAG
GCCAGCAGCAGAAGCAGCUCUAGAAGCAGAGGCAACAGCCGGAAUAGCACCCCUGGCAGCUCCAGAGGCAAUUCCCCU
GCCAGAAUGGCUUCUGGCGGCGGAGAAACAGCUCUGGCACUGCUGCUGCUCGACCGGCUGAAUCAGCUGGAAUCUAAG
GUGUCCGGCAAGGGCCAGCAACAGCAGGGACAGACCGUGACCAAGAAGUCUGCCGCUGAGGCCAGCAAGAAGCCCAGA
CAGAAGAGAACCGCCACCAAGCAGUACAACGUGACCCAGGCCUUUGGCAGAAGAGGCCCAGAACAGACCCAGGGCAAU
UUCGGCGACCAGGACCUGAUCAGACAGGGCACCGAUUACAAGCACUGGCCCCAGAUCGCCCAGUUUGCCCCUUCUGCC
UCUGCCUUUUUCGGCAUGAGCCGGAUCGGCAUGGAAGUGACACCUAGCGGCACCUGGCUGACAUAUCACGGCGCCAUC
AAGCUGGACGACAAGGACCCUCAGUUCAAGGACAACGUGAUCCUGCUGAACAAGCACAUCGACGCCUACAAGACAUUC
CCUCCAACCGAGCCUAAGAAGGACAAGAAGAAGAAAACCGACGAGGCCCAGCCUCUGCCACAGAGACAGAAAAAGCAG
CCCACCGUGACACUGCUGCCUGCCGCCGAUAUGGACGACUUCUCUAGACAGCUGCAGAACAGCAUGAGCGGCGCCAGC
GCUGAUUCUACACAAGCU

Hence, preferably the RNA construct comprises a sequence substantially as set out in SEQ ID No: 238, or a fragment or variant thereof.

In one embodiment, the at least one IIP is MHV N protein (P03416; Nucleoprotein Murine coronavirus (strain A59)), or an orthologue thereof. One embodiment of the polypeptide sequence of MHV N protein is represented herein as SEQ ID No: 239, as so follows:

[SEQ ID No: 239]
MSFVPGQENAGGRSSSVNRAGNGILKKTTWADQTERGPNNQNRGRRNQPKQTATTQPNSGSVVPHYSWFSGITQFQKG
KEFQFAEGQGVPIANGIPASEQKGYWYRHNRRSFKTPDGQQKQLLPRWYFYYLGTGPHAGASYGDSIEGVFWVANSQA
DTNTRSDIVERDPSSHEAIPTRFAPGTVLPQGFYVEGSGRSAPASRSGSRSQSRGPNNRARSSSNQRQPASTVKPDMA
EEIAALVLAKLGKDAGQPKQVTKQSAKEVROKILNKPRQKRTPNKQCPVQQCFGKRGPNQNFGGSEMLKLGTSDPQFP
ILAELAPTVGAFFFGSKLELVKKNSGGADEPTKDVYELQYSGAVRFDSTLPGFETIMKVLNENLNAYQKDGGADVVSP
KPQRKGRRQAQEKKDEVDNVSVAKPKSSVQRNVSRELTPEDRSLLAQILDDGVVPDGLEDDSNV

Therefore, preferably the RNA construct comprises a nucleotide sequence which encodes an amino acid sequence substantially as set out in SEQ ID No: 239, or a variant or fragment thereof.

In one embodiment, the MHV N polypeptide is encoded by the DNA nucleotide sequence of SEQ ID No: 240, as follows:

[SEQ ID No: 240]
ATGTCTTTTGTTCCTGGGCAAGAAAATGCCGGTGGCAGAAGCTCCTCTGTAAACCGCGCTGGTAATGGAATCCTCAAG
AAGACCACTTGGGCTGACCAAACCGAGCGTGGACCAAATAATCAAAATAGAGGCAGAAGGAATCAGCCAAAGCAGACT
GCAACTACTCAACCCAACTCCGGGAGTGTGGTTCCCCATTACTCCTGGTTTTCTGGCATTACCCAGTTCCAAAAGGGA
AAGGAGTTTCAGTTTGCAGAAGGACAAGGAGTGCCTATTGCCAATGGAATCCCCGCTTCAGAGCAAAAGGGATATTGG
TATAGACACAACCGCCGTTCTTTTAAAACACCTGATGGGCAGCAGAAGCAATTACTGCCCAGATGGTATTTTTACTAT
CTTGGCACAGGGCCCCATGCTGGAGCCAGTTATGGAGACAGCATTGAAGGTGTCTTCTGGGTTGCAAACAGCCAAGCG
GACACCAATACCCGCTCTGATATTGTCGAAAGGGACCCAAGCAGTCATGAGGCTATTCCTACTAGGTTTGCGCCCGGC
ACGGTATTGCCTCAGGGCTTTTATGTTGAAGGCTCTGGAAGGTCTGCACCTGCTAGCCGATCTGGTTCGCGGTCACAA
TCCCGTGGGCCAAATAATCGCGCTAGAAGCAGTTCCAACCAGCGCCAGCCTGCCTCTACTGTAAAACCTGATATGGCC
GAAGAAATTGCTGCTCTTGTTTTGGCTAAGCTCGGTAAAGATGCCGGCCAGCCCAAGCAAGTAACGAAGCAAAGTGCC
AAAGAAGTCAGGCAGAAAATTTTAAACAAGCCTCGCCAAAAGAGGACTCCAAACAAGCAGTGCCCAGTGCAGCAGTGT
TTTGGAAAGAGAGGCCCCAATCAGAATTTTGGAGGCTCTGAAATGTTAAAACTTGGAACTAGTGATCCACAGTTCCCC
ATTCTTGCAGAGTTGGCTCCAACAGTTGGTGCCTTCTTCTTTGGATCTAAATTAGAATTGGTCAAAAAGAATTCTGGT
GGTGCTGATGAACCCACCAAAGATGTGTATGAGCTGCAATATTCAGGTGCAGTTAGATTTGATAGTACTCTACCTGGT
TTTGAGACTATCATGAAAGTGTTGAATGAGAATTTGAATGCCTACCAGAAGGATGGTGGTGCAGATGTGGTGAGCCCA
AAGCCCCAAAGAAAAGGGCGTAGACAGGCTCAGGAAAAGAAAGATGAAGTAGATAATGTAAGCGTTGCAAAGCCCAAA
AGCTCTGTGCAGCGAAATGTAAGTAGAGAATTAACCCCAGAGGATAGAAGTCTGTTGGCTCAGATCCTTGATGATGGC
GTAGTGCCAGATGGGTTAGAAGATGACTCTAATGTG

Accordingly, preferably the MHV N polypeptide is encoded by the DNA nucleotide sequence substantially as set out in SEQ ID NO: 240, or a variant or fragment thereof.

One embodiment of codon optimised nucleic acid (DNA) encoding the MHV N polypeptide is provided herein as SEQ ID No: 241, as follows:

ATGAGCTTCGTGCCCGGCCAAGAAAATGCCGGCGGAAGATCTAGCAGCGTGAACAGAGCCGGCAACGGCATCCTGAAG
AAAACCACCTGGGCCGACCAGACCGAGAGAGGCCCCAACAACCAGAACCGGGGCAGAAGAAACCAGCCTAAGCAGACC
GCCACCACACAGCCTAATAGCGGCTCTGTGGTGCCCCACTACAGCTGGTTTAGCGGCATCACCCAGTTCCAGAAGGGC
AAAGAGTTCCAGTTCGCCGAAGGACAGGGCGTGCCAATCGCCAATGGAATCCCTGCCTCTGAGCAGAAAGGCTACTGG
TACAGACACAACCGGCGGAGCTTCAAGACCCCTGATGGACAGCAGAAACAGCTGCTGCCCAGATGGTACTTCTACTAC
CTCGGCACAGGACCTCACGCAGGCGCCTCTTATGGCGATTCTATCGAGGGCGTGTTCTGGGTCGCCAACAGCCAGGCC
GATACCAACACCAGATCCGACATCGTGGAACGGGACCCTAGCAGCCACGAGGCCATTCCTACAAGATTTGCCCCTGGC
ACCGTGCTGCCTCAGGGCTTTTATGTGGAAGGCAGCGGAAGAAGCGCCCCTGCCAGTAGATCTGGCAGCAGATCTCAG
AGCAGGGGCCCTAACAACAGAGCCAGAAGCAGCAGCAACCAGAGACAGCCTGCCAGCACCGTGAAGCCCGATATGGCC
GAAGAAATCGCCGCTCTGGTGCTGGCCAAGCTGGGAAAAGATGCCGGCCAGCCAAAGCAAGTGACCAAGCAGAGCGCC
AAAGAAGTGCGGCAGAAGATCCTGAACAAGCCCCGGCAGAAGCGGACCCCTAACAAGCAGTGTCCTGTGCAGCAGTGC
TTCGGCAAGAGGGGCCCCAATCAGAATTTTGGCGGCAGCGAGATGCTGAAGCTGGGCACAAGCGATCCTCAGTTCCCT
ATCCTGGCCGAGCTGGCTCCTACAGTGGGCGCATTTTTCTTTGGCTCCAAACTCGAGCTGGTCAAGAAGAACAGCGGC
GGAGCCGATGAGCCCACCAAGGATGTGTACGAGCTGCAGTACTCTGGCGCCGTCAGATTCGATAGCACCCTGCCTGGC
TTCGAGACAATCATGAAGGTGCTGAACGAGAACCTGAACGCCTACCAGAAGGATGGCGGCGCTGACGTGGTGTCTCCT
AAGCCTCAGAGAAAAGGCAGACGGCAGGCCCAAGAGAAGAAAGACGAGGTGGACAACGTGTCCGTGGCCAAGCCTAAG

[SEQ ID No: 241]
AGCAGCGTGCAGAGAAACGTGTCCAGAGAGCTGACCCCAGAGGACAGATCTCTGCTGGCTCAGATCCTGGACGATGGC
GTGGTGCCAGATGGCCTGGAAGATGACAGCAACGTG

Hence, preferably the RNA construct is encoded by a DNA sequence substantially as set out in SEQ ID No: 241, or a fragment or variant thereof.

In an embodiment, the RNA sequence corresponding to the codon optimised DNA sequence of SEQ ID No: 241 is provided herein as SEQ ID No: 242, as follows:

[SEQ ID No: 242]
AUGAGCUUCGUGCCCGGCCAAGAAAAUGCCGGCGGAAGAUCUAGCAGCGUGAACAGAGCCGGCAACGGCAUCCUGAAG
AAAACCACCUGGGCCGACCAGACCGAGAGAGGCCCCAACAACCAGAACCGGGGCAGAAGAAACCAGCCUAAGCAGACC
GCCACCACACAGCCUAAUAGCGGCUCUGUGGUGCCCCACUACAGCUGGUUUAGCGGCAUCACCCAGUUCCAGAAGGGC
AAAGAGUUCCAGUUCGCCGAAGGACAGGGCGUGCCAAUCGCCAAUGGAAUCCCUGCCUCUGAGCAGAAAGGCUACUGG
UACAGACACAACCGGCGGAGCUUCAAGACCCCUGAUGGACAGCAGAAACAGCUGCUGCCCAGAUGGUACUUCUACUAC
CUCGGCACAGGACCUCACGCAGGCGCCUCUUAUGGCGAUUCUAUCGAGGGCGUGUUCUGGGUCGCCAACAGCCAGGCC
GAUACCAACACCAGAUCCGACAUCGUGGAACGGGACCCUAGCAGCCACGAGGCCAUUCCUACAAGAUUUGCCCCUGGC
ACCGUGCUGCCUCAGGGCUUUUAUGUGGAAGGCAGCGGAAGAAGCGCCCCUGCCAGUAGAUCUGGCAGCAGAUCUCAG
AGCAGGGGCCCUAACAACAGAGCCAGAAGCAGCAGCAACCAGAGACAGCCUGCCAGCACCGUGAAGCCCGAUAUGGCC
GAAGAAAUCGCCGCUCUGGUGCUGGCCAAGCUGGGAAAAGAUGCCGGCCAGCCAAAGCAAGUGACCAAGCAGAGCGCC
AAAGAAGUGCGGCAGAAGAUCCUGAACAAGCCCCGGCAGAAGCGGACCCCUAACAAGCAGUGUCCUGUGCAGCAGUGC
UUCGGCAAGAGGGGCCCCAAUCAGAAUUUUGGCGGCAGCGAGAUGCUGAAGCUGGGCACAAGCGAUCCUCAGUUCCCU
AUCCUGGCCGAGCUGGCUCCUACAGUGGGCGCAUUUUUCUUUGGCUCCAAACUCGAGCUGGUCAAGAAGAACAGCGGC
GGAGCCGAUGAGCCCACCAAGGAUGUGUACGAGCUGCAGUACUCUGGCGCCGUCAGAUUCGAUAGCACCCUGCCUGGC
UUCGAGACAAUCAUGAAGGUGCUGAACGAGAACCUGAACGCCUACCAGAAGGAUGGCGGCGCUGACGUGGUGUCUCCU
AAGCCUCAGAGAAAAGGCAGACGGCAGGCCCAAGAGAAGAAAGACGAGGUGGACAACGUGUCCGUGGCCAAGCCUAAG
AGCAGCGUGCAGAGAAACGUGUCCAGAGAGCUGACCCCAGAGGACAGAUCUCUGCUGGCUCAGAUCCUGGACGAUGGC
GUGGUGCCAGAUGGCCUGGAAGAUGACAGCAACGUG

Hence, preferably the RNA construct comprises a sequence substantially as set out in SEQ ID No: 242, or a fragment or variant thereof.

In one embodiment, the at least one IIP is FMDV 2B protein (P03307; Protease 3C Genome polyprotein foot-and-mouth disease virus (isolate-/Germany/A5Westerwald/1951 serotype A), or an orthologue thereof. One embodiment of the polypeptide sequence of FMDV 2B protein is represented herein as SEQ ID No: 243, as follows:

[SEQ ID No: 243]
PFFFSDVRSNFSKLVETINQMQEDMSTKHGPDFNRLVSAFEELAAGVKAIRTGLDEAKPWYKLIKLLSRLSCMAAVAA
RSKDPVLVAIMLADTGLEILDSTFVVKKISDSLSSLFHVPAPVFSFGAPILLAGLVKVASSFFRSTPEDLERAEKQ

Therefore, preferably the RNA construct comprises a nucleotide sequence which encodes an amino acid sequence substantially as set out in SEQ ID No: 243, or a variant or fragment thereof.

In one embodiment, the FMDV 2B polypeptide is encoded by the DNA nucleotide sequence of SEQ ID No: 244, as follows:

[SEQ ID No: 244]
CCCTTCTTCTTCTCTGACGTTAGGTCAAACTTTTCTAAGCTGGTGGAAACCATCAACCAGATGCAGGAAGACATGTCA
ACAAAACACGGGCCCGACTTTAACCGGTTGGTGTCCGCCTTTGAGGAACTGGCCGCTGGAGTAAAAGCCATCAGGACC
GGCCTCGACGAGGCCAAACCCTGGTACAAGCTTATCAAACTCCTAAGCCGCCTGTCGTGCATGGCCGCTGTGGCAGCA
CGGTCCAAGGACCCAGTCCTTGTGGCCATCATGCTGGCCGACACCGGTCTCGAGATTCTGGACAGCACTTTCGTCGTG
AAGAAGATCTCCGACTCGCTCTCCAGTCTCTTCCACGTGCCGGCCCCCGTCTTCAGTTTCGGAGCCCCGATTCTGCTA
GCCGGGCTGGTCAAGGTCGCCTCGAGTTTCTTCCGGTCCACGCCCGAAGACCTTGAGAGAGCAGAGAAACAG

Accordingly, preferably the FMDV 2B polypeptide is encoded by the DNA nucleotide sequence substantially as set out in SEQ ID NO: 244, or a variant or fragment thereof.

One embodiment of codon optimised nucleic acid (DNA) encoding the FMDV 2B polypeptide is provided herein as SEQ ID No: 245, as follows:

[SEQ ID No: 245]
CCGTTCTTCTTTAGCGACGTGCGGAGCAACTTCAGCAAGCTGGTGGAAACCATCAACCAGATGCAAGAGGACATGAGC
ACCAAGCACGGCCCCGACTTCAACAGACTGGTGTCCGCCTTTGAGGAACTGGCCGCTGGCGTGAAGGCCATCAGAACA
GGACTGGATGAGGCCAAGCCTTGGTACAAGCTGATCAAGCTGCTGAGCCGGCTGAGCTGTATGGCTGCTGTGGCCGCC
AGATCCAAGGATCCTGTGCTGGTGGCCATCATGCTGGCCGATACAGGCCTGGAAATCCTGGACAGCACCTTCGTGGTC
AAGAAGATCAGCGACAGCCTGAGCAGCCTGTTCCACGTGCCAGCTCCAGTGTTCTCTTTTGGCGCCCCTATTCTGCTG
GCCGGCCTGGTCAAAGTGGCCAGCAGCTTCTTTAGAAGCACCCCTGAGGACCTGGAACGGGCCGAAAAACAA

Hence, preferably the RNA construct is encoded by a DNA sequence substantially as set out in SEQ ID No: 245, or a fragment or variant thereof.

In an embodiment, the RNA sequence corresponding to the codon optimised DNA sequence of SEQ ID No: 245 is provided herein as SEQ ID No: 246, as follows:

[SEQ ID No: 246]
CCGUUCUUCUUUAGCGACGUGCGGAGCAACUUCAGCAAGCUGGUGGAAACCAUCAACCAGAUGCAAGAGGACAUGAGC
ACCAAGCACGGCCCCGACUUCAACAGACUGGUGUCCGCCUUUGAGGAACUGGCCGCUGGCGUGAAGGCCAUCAGAACA
GGACUGGAUGAGGCCAAGCCUUGGUACAAGCUGAUCAAGCUGCUGAGCCGGCUGAGCUGUAUGGCUGCUGUGGCCGCC
AGAUCCAAGGAUCCUGUGCUGGUGGCCAUCAUGCUGGCCGAUACAGGCCUGGAAAUCCUGGACAGCACCUUCGUGGUC
AAGAAGAUCAGCGACAGCCUGAGCAGCCUGUUCCACGUGCCAGCUCCAGUGUUCUCUUUUGGCGCCCCUAUUCUGCUG
GCCGGCCUGGUCAAAGUGGCCAGCAGCUUCUUUAGAAGCACCCCUGAGGACCUGGAACGGGCCGAAAAACAA

Hence, preferably the RNA construct comprises a sequence substantially as set out in SEQ ID No: 246, or a fragment or variant thereof.

In one embodiment, the at least one IIP is Influenza virus A PB1-F2 (B4URE5; Protein PB1-F2 Influenza A virus (strain A/Russia:St.Petersburg/8/2006 H1N1), or an orthologue thereof. One embodiment of the polypeptide sequence of Influenza virus A PB1-F2 is represented herein as SEQ ID No: 247, as follows:

[SEQ ID No: 247]
MGQEQDTPWILSTGHISTQKREDGQQTPKLEHRNSTRLMGHFQKTMNQVVMPKQIVYWRRWLSLRNPILVFLKTRVLK
RWRLFSKHE

Therefore, preferably the RNA construct comprises a nucleotide sequence which encodes an amino acid sequence substantially as set out in SEQ ID No: 247, or a variant or fragment thereof.

In one embodiment, the Influenza virus A PB1-F2 polypeptide is encoded by the DNA nucleotide sequence of SEQ ID No: 248, as follows:

[SEQ ID No: 248]
ATGGGACAGGAACAGGATACACCATGGATACTGTCAACAGGACACATCAGTACTCAGAAAAGGGAAGATGGACAACAA
ACACCGAAACTGGAGCACCGCAACTCAACCCGATTGATGGGCCACTTCCAGAAGACAATGAACCAAGTGGTTATGCCC
AAACAGATTGTGTATTGGAGGCGATGGCTTTCCTTGAGGAATCCCATCCTGGTATTTTTGAAAACTCGTGTATTGAAA
CGATGGAGGTTGTTCAGCAAACACGAG

Accordingly, preferably the Influenza virus A PB1-F2 polypeptide is encoded by the DNA nucleotide sequence substantially as set out in SEQ ID NO: 248, or a variant or fragment thereof.

One embodiment of codon optimised nucleic acid (DNA) encoding the Influenza virus A PB1-F2 polypeptide is provided herein as SEQ ID No: 249, as follows:

[SEQ ID No: 249]
ATGGGCCAAGAGCAGGACACACCTTGGATCCTGAGCACCGGCCACATCAGCACCCAGAAGAGAGAGGACGGACAGCAG
ACCCCTAAGCTGGAACACCGGAACAGCACCAGACTGATGGGCCACTTCCAGAAAACCATGAACCAGGTGGTCATGCCC
AAGCAGATCGTGTACTGGCGGAGATGGCTGAGCCTGCGGAATCCTATCCTGGTGTTCCTGAAAACCCGGGTGCTGAAG
AGATGGCGGCTGTTCTCTAAGCACGAG

Hence, preferably the RNA construct is encoded by a DNA sequence substantially as set out in SEQ ID No: 249, or a fragment or variant thereof.

In an embodiment, the RNA sequence corresponding to the codon optimised DNA sequence of SEQ ID No: 249 is provided herein as SEQ ID No: 250, as follows:

[SEQ ID No: 250]
AUGGGCCAAGAGCAGGACACACCUUGGAUCCUGAGCACCGGCCACAUCAGCACCCAGAAGAGAGAGGACGGACAGCAG
ACCCCUAAGCUGGAACACCGGAACAGCACCAGACUGAUGGGCCACUUCCAGAAAACCAUGAACCAGGUGGUCAUGCCC
AAGCAGAUCGUGUACUGGCGGAGAUGGCUGAGCCUGCGGAAUCCUAUCCUGGUGUUCCUGAAAACCCGGGUGCUGAAG
AGAUGGCGGCUGUUCUCUAAGCACGAG

Hence, preferably the RNA construct comprises a sequence substantially as set out in SEQ ID No: 250, or a fragment or variant thereof.

In one embodiment, the at least one IIP is Hepatitis A Protein 3ABC (Q05794; Genome polyprotein Human hepatitis A virus genotype IA (isolate HAS-15)), or an orthologue thereof. One embodiment of the polypeptide sequence of Hepatitis A Protein 3ABC is represented herein as SEQ ID No: 251, as follows:

[SEQ ID No: 251]
GISDDDSAVAEFFQSFPSGEPSNSKLSSFFQSVTNHKWVAVGAAVGILGLLVGGWFVYKHFSRKEEEPIPAEGVYHGV
TKPKQVIKLDADPVESQSTLEIAGLVRKNLVQFGVGEKNGCVRWVMNALGVKDDWLLVPSHAYKFEKDYEMMEFYFNR
GGTYYSISAGNVVIQSLDVGFQDVVLMKVPTIPKFRDITQHFIKKGDVPRALNRLATLVTTVNGTPMLISEGPLKMEE
KATYVHKKNDGTTVDLTVDQAWRGKGEGLPGMCGGALVSSNQSIQNAILGIHVAGGNSILVAKLITQEMFQNIDKKIE
SQ

Therefore, preferably the RNA construct comprises a nucleotide sequence which encodes an amino acid sequence substantially as set out in SEQ ID No: 251, or a variant or fragment thereof.

In one embodiment, the Hepatitis A Protein 3ABC polypeptide is encoded by the DNA nucleotide sequence of SEQ ID No: 252, as follows:

[SEQ ID No: 252]
GGAATTTCAGATGATGACAGTGCAGTAGCTGAGTTTTTCCAGTCTTTTCCATCTGGTGAACCATCAAATTCCAAGTTA
TCTAGTTTTTTCCAATCTGTCACTAATCACAAGTGGGTTGCTGTGGGAGCTGCAGTTGGTATTCTTGGATTGCTAGTG
GGAGGATGGTTTGTGTATAAGCATTTTTCCCGCAAAGAGGAAGAACCAATTCCAGCTGAAGGGGTTTATCATGGAGTG
ACTAAGCCCAAACAAGTGATTAAATTGGATGCAGATCCAGTAGAGTCCCAGTCAACTCTAGAAATAGCAGGATTAGTT
AGGAAAAATTTGGTTCAGTTTGGAGTTGGTGAGAAAAATGGATGTGTGAGATGGGTCATGAATGCCTTAGGAGTGAAG
GATGATTGGTTGTTAGTACCTTCTCATGCTTATAAATTTGAAAAGGATTATGAAATGATGGAGTTTTATTTCAATAGA
GGTGGAACTTACTATTCAATTTCAGCTGGTAATGTTGTTATTCAATCTTTAGATGTGGGATTCCAAGATGTTGTTCTA
ATGAAGGTTCCTACAATTCCCAAGTTTAGAGATATTACTCAACATTTTATTAAGAAAGGAGATGTGCCTAGAGCCTTG
AATCGCTTGGCAACATTAGTGACAACCGTTAATGGAACTCCTATGTTAATTTCTGAGGGACCTTTAAAAATGGAAGAA
AAAGCCACTTATGTTCATAAGAAGAACGATGGTACTACGGTTGATTTGACTGTAGATCAGGCATGGAGAGGAAAAGGT
GAAGGTCTTCCTGGAATGTGTGGTGGGGCCCTAGTGTCATCAAATCAGTCCATACAAAATGCAATTTTGGGTATTCAT
GTTGCTGGAGGAAATTCAATTCTTGTGGCAAAGTTGATTACTCAAGAAATGTTTCAAAACATTGATAAGAAAATTGAA
AGTCAG

Accordingly, preferably the Hepatitis A Protein 3ABC polypeptide is encoded by the DNA nucleotide sequence substantially as set out in SEQ ID NO: 252, or a variant or fragment thereof.

One embodiment of codon optimised nucleic acid (DNA) encoding the Hepatitis A Protein 3ABC polypeptide is provided herein as SEQ ID No: 253, as follows:

[SEQ ID No: 253]
GGCATCAGCGACGATGATTCTGCCGTGGCCGAGTTCTTCCAGAGCTTTCCTAGCGGCGAGCCCAGCAACAGCAAGCTG
AGCAGCTTCTTCCAGTCCGTGACCAACCACAAATGGGTCGCCGTGGGAGCCGCTGTGGGAATTCTGGGACTTCTCGTT
GGCGGATGGTTCGTGTACAAGCACTTCAGCCGGAAAGAGGAAGAACCCATTCCTGCCGAGGGCGTGTACCACGGCGTG
ACCAAACCTAAGCAAGTGATCAAGCTGGACGCCGATCCTGTGGAAAGCCAGAGCACACTGGAAATCGCCGGACTCGTG
CGGAAGAACCTGGTGCAGTTTGGCGTGGGCGAGAAGAACGGCTGTGTCAGATGGGTCATGAACGCCCTGGGCGTGAAG
GACGATTGGCTGCTGGTTCCTAGCCACGCCTACAAGTTCGAGAAGGACTACGAGATGATGGAATTCTACTTCAACAGA
GGCGGCACCTACTACAGCATCAGCGCCGGCAATGTGGTCATCCAGTCTCTGGATGTGGGCTTCCAGGACGTGGTGCTG
ATGAAGGTGCCAACAATCCCCAAGTTCCGGGACATCACCCAGCACTTCATCAAGAAAGGCGACGTGCCCAGGGCTCTG
AACAGACTGGCTACCCTGGTCACCACCGTGAACGGCACACCCATGCTGATCTCTGAGGGCCCACTGAAGATGGAAGAG
AAGGCCACCTACGTGCACAAGAAGAACGACGGCACCACAGTGGACCTGACCGTGGATCAAGCTTGGAGAGGCAAAGGC
GAGGGCCTGCCTGGAATGTGTGGCGGAGCACTGGTGTCCAGCAACCAGAGCATCCAGAATGCCATCCTGGGCATCCAT
GTGGCTGGCGGCAATTCTATCCTGGTGGCCAAGCTGATCACCCAAGAGATGTTCCAGAACATCGACAAGAAGATCGAG
AGCCAG

Hence, preferably the RNA construct is encoded by a DNA sequence substantially as set out in SEQ ID No: 253, or a fragment or variant thereof.

In an embodiment, the RNA sequence corresponding to the codon optimised DNA sequence of SEQ ID No: 253 is provided herein as SEQ ID No: 254, as follows:

[SEQ ID No: 254]
GGCAUCAGCGACGAUGAUUCUGCCGUGGCCGAGUUCUUCCAGAGCUUUCCUAGCGGCGAGCCCAGCAACAGCAAGCUG
AGCAGCUUCUUCCAGUCCGUGACCAACCACAAAUGGGUCGCCGUGGGAGCCGCUGUGGGAAUUCUGGGACUUCUCGUU
GGCGGAUGGUUCGUGUACAAGCACUUCAGCCGGAAAGAGGAAGAACCCAUUCCUGCCGAGGGCGUGUACCACGGCGUG
ACCAAACCUAAGCAAGUGAUCAAGCUGGACGCCGAUCCUGUGGAAAGCCAGAGCACACUGGAAAUCGCCGGACUCGUG
CGGAAGAACCUGGUGCAGUUUGGCGUGGGCGAGAAGAACGGCUGUGUCAGAUGGGUCAUGAACGCCCUGGGCGUGAAG
GACGAUUGGCUGCUGGUUCCUAGCCACGCCUACAAGUUCGAGAAGGACUACGAGAUGAUGGAAUUCUACUUCAACAGA
GGCGGCACCUACUACAGCAUCAGCGCCGGCAAUGUGGUCAUCCAGUCUCUGGAUGUGGGCUUCCAGGACGUGGUGCUG
AUGAAGGUGCCAACAAUCCCCAAGUUCCGGGACAUCACCCAGCACUUCAUCAAGAAAGGCGACGUGCCCAGGGCUCUG
AACAGACUGGCUACCCUGGUCACCACCGUGAACGGCACACCCAUGCUGAUCUCUGAGGGCCCACUGAAGAUGGAAGAG
AAGGCCACCUACGUGCACAAGAAGAACGACGGCACCACAGUGGACCUGACCGUGGAUCAAGCUUGGAGAGGCAAAGGC
GAGGGCCUGCCUGGAAUGUGUGGCGGAGCACUGGUGUCCAGCAACCAGAGCAUCCAGAAUGCCAUCCUGGGCAUCCAU
GUGGCUGGCGGCAAUUCUAUCCUGGUGGCCAAGCUGAUCACCCAAGAGAUGUUCCAGAACAUCGACAAGAAGAUCGAG
AGCCAG

Hence, preferably the RNA construct comprises a sequence substantially as set out in SEQ ID No: 254, or a fragment or variant thereof.

In one embodiment, the at least one IIP is Hepatitis B X protein (P03165; Protein X Hepatitis B virus genotype D), or an orthologue thereof. One embodiment of the polypeptide sequence of Hepatitis B X protein is represented herein as SEQ ID No: 255, as follows:

[SEQ ID No: 255]
MAARLCCQLDPARDVLCLRPVGAESRGRPFSGSLGTLSSPSPSAVSTDHG
AHLSLRGLPVCAFSSAGPCALRFTSARRMETTVKAQPFLPKVLHKRTLGL
SVMSTTDLEAYFKDCLFKDWEELGEEIRLKVFVLGGCRHKLVCAPAPCNF
FTSA

Therefore, preferably the RNA construct comprises a nucleotide sequence which encodes an amino acid sequence substantially as set out in SEQ ID No: 255, or a variant or fragment thereof.

In one embodiment, the Hepatitis B X polypeptide is encoded by the DNA nucleotide sequence of SEQ ID No: 256, as follows:

[SEQ ID No: 256]
ATGGCTGCTAGGCTGTGCTGCCAACTGGATCCTGCGCGGGACGTCCTTTG
TTTACGTCCCGTCGGCGCTGAATCCCGCGGACGACCCTTCTCGGGGTCGC
TTGGGACTCTCTCGTCCCCTTCTCCGTCTGCCGTTTCGACCGACCACGGG
GCGCACCTCTCTTTACGCGGACTCCCCGTCTGTGCCTTCTCATCTGCCGG
ACCGTGTGCACTTCGCTTCACCTCTGCACGTCGCATGGAGACCACCGTGA
AAGCCCAACCATTCTTGCCCAAGGTCTTACATAAGAGGACTCTTGGACTC
TCTGTAATGTCAACGACCGACCTTGAGGCATACTTCAAAGACTGTTTGTT
TAAAGACTGGGAGGAGTTGGGGGAGGAGATTAGATTAAAGGTCTTTGTAT
TAGGAGGCTGTAGGCATAAATTGGTCTGCGCACCAGCACCATGCAACTTT
TTCACCTCTGCC

Accordingly, preferably the Hepatitis B X polypeptide is encoded by the DNA nucleotide sequence substantially as set out in SEQ ID NO: 256, or a variant or fragment thereof.

One embodiment of codon optimised nucleic acid (DNA) encoding the Hepatitis B X polypeptide is provided herein as SEQ ID No: 257, as follows:

[SEQ ID No: 257]
ATGGCCGCCAGACTGTGCTGTCAACTGGACCCTGCTAGGGACGTGCTGTG
TCTCAGACCTGTGGGAGCCGAGTCTAGAGGCAGACCTTTTTCTGGCTCTC
TGGGCACCCTGAGCAGCCCATCTCCATCTGCCGTGTCTACAGATCACGGC
GCCCACCTGTCTCTGAGAGGACTGCCTGTGTGTGCCTTTAGCAGCGCCGG
ACCTTGCGCTCTGAGATTCACATCTGCCAGACGGATGGAAACCACCGTGA
AGGCCCAGCCTTTCCTGCCTAAGGTGCTGCACAAGAGAACCCTGGGCCTG
AGCGTGATGAGCACCACAGATCTGGAAGCCTACTTCAAGGATTGCCTGTT
CAAGGACTGGGAAGAACTGGGCGAAGAGATCCGGCTGAAGGTGTTCGTGC
TCGGCGGATGCAGACACAAGCTCGTGTGTGCTCCCGCTCCTTGCAACTTC
TTTACCAGCGCT

Hence, preferably the RNA construct is encoded by a DNA sequence substantially as set out in SEQ ID No: 257, or a fragment or variant thereof.

In an embodiment, the RNA sequence corresponding to the codon optimised DNA sequence of SEQ ID No: 257 is provided herein as SEQ ID No: 258, as follows:

[SEQ ID No: 258]
AUGGCCGCCAGACUGUGCUGUCAACUGGACCCUGCUAGGGACGUGCUGUG
UCUCAGACCUGUGGGAGCCGAGUCUAGAGGCAGACCUUUUUCUGGCUCUC
UGGGCACCCUGAGCAGCCCAUCUCCAUCUGCCGUGUCUACAGAUCACGGC
GCCCACCUGUCUCUGAGAGGACUGCCUGUGUGUGCCUUUAGCAGCGCCGG
ACCUUGCGCUCUGAGAUUCACAUCUGCCAGACGGAUGGAAACCACCGUGA
AGGCCCAGCCUUUCCUGCCUAAGGUGCUGCACAAGAGAACCCUGGGCCUG
AGCGUGAUGAGCACCACAGAUCUGGAAGCCUACUUCAAGGAUUGCCUGUU
CAAGGACUGGGAAGAACUGGGCGAAGAGAUCCGGCUGAAGGUGUUCGUGC
UCGGCGGAUGCAGACACAAGCUCGUGUGUGCUCCCGCUCCUUGCAACUUC
UUUACCAGCGCU

Hence, preferably the RNA construct comprises a sequence substantially as set out in SEQ ID No: 258, or a fragment or variant thereof.

In one embodiment, the at least one IIP is SARS-CoV NSP15 (P0C6X7; Replicase polyprotein Severe acute respiratory syndrome coronavirus), or an orthologue thereof. One embodiment of the polypeptide sequence of SARS-CoV NSP15 is represented herein as SEQ ID No: 259, as follows:

[SEQ ID No: 259]
SLENVAYNVVNKGHFDGHAGEAPVSIINNAVYTKVDGIDVEIFENKTTLP
VNVAFELWAKRNIKPVPEIKILNNLGVDIAANTVIWDYKREAPAHVSTIG
VCTMTDIAKKPTESACSSLTVLFDGRVEGQVDLFRNARNGVLITEGSVKG
LTPSKGPAQASVNGVTLIGESVKTQFNYFKKVDGIIQQLPETYFTQSRDL
EDFKPRSQMETDFLELAMDEFIQRYKLEGYAFEHIVYGDFSHGQLGGLHL
MIGLAKRSQDSPLKLEDFIPMDSTVKNYFITDAQTGSSKCVCSVIDLLLD
DFVEIIKSQDLSVISKVVKVTIDYAEISFMLWCKDGHVETFYPKLQ

Therefore, preferably the RNA construct comprises a nucleotide sequence which encodes an amino acid sequence substantially as set out in SEQ ID No: 259, or a variant or fragment thereof.

In one embodiment, the SARS-CoV NSP15 polypeptide is encoded by the DNA nucleotide sequence of SEQ ID No: 260, as follows:

[SEQ ID No: 260]
AGTTTAGAAAATGTGGCTTATAATGTTGTTAATAAAGGACACTTTGATGG
ACACGCCGGCGAAGCACCTGTTTCCATCATTAATAATGCTGTTTACACAA
AGGTAGATGGTATTGATGTGGAGATCTTTGAAAATAAGACAACACTTCCT
GTTAATGTTGCATTTGAGCTTTGGGCTAAGCGTAACATTAAACCAGTGCC
AGAGATTAAGATACTCAATAATTTGGGTGTTGATATCGCTGCTAATACTG
TAATCTGGGACTACAAAAGAGAAGCCCCAGCACATGTATCTACAATAGGT
GTCTGCACAATGACTGACATTGCCAAGAAACCTACTGAGAGTGCTTGTTC
TTCACTTACTGTCTTGTTTGATGGTAGAGTGGAAGGACAGGTAGACCTTT
TTAGAAACGCCCGTAATGGTGTTTTAATAACAGAAGGTTCAGTCAAAGGT
CTAACACCTTCAAAGGGACCAGCACAAGCTAGCGTCAATGGAGTCACATT
AATTGGAGAATCAGTAAAAACACAGTTTAACTACTTTAAGAAAGTAGACG
GCATTATTCAACAGTTGCCTGAAACCTACTTTACTCAGAGCAGAGACTTA
GAGGATTTTAAGCCCAGATCACAAATGGAAACTGACTTTCTCGAGCTCGC
TATGGATGAATTCATACAGCGATATAAGCTCGAGGGCTATGCCTTCGAAC
ACATCGTTTATGGAGATTTCAGTCATGGACAACTTGGCGGTCTTCATTTA
ATGATAGGCTTAGCCAAGCGCTCACAAGATTCACCACTTAAATTAGAGGA
TTTTATCCCTATGGACAGCACAGTGAAAAATTACTTCATAACAGATGCGC
AAACAGGTTCATCAAAATGTGTGTGTTCTGTGATTGATCTTTTACTTGAT
GACTTTGTCGAGATAATAAAGTCACAAGATTTGTCAGTGATTTCAAAAGT
GGTCAAGGTTACAATTGACTATGCTGAAATTTCATTCATGCTTTGGTGTA
AGGATGGACATGTTGAAACCTTCTACCCAAAACTACAA

Accordingly, preferably the SARS-CoV NSP15 polypeptide is encoded by the DNA nucleotide sequence substantially as set out in SEQ ID NO: 260, or a variant or fragment thereof.

One embodiment of codon optimised nucleic acid (DNA) encoding the SARS-CoV NSP15 polypeptide is provided herein as SEQ ID No: 261, as follows:

[SEQ ID No: 261]
AGCCTGGAAAACGTGGCCTACAACGTGGTCAACAAGGGCCACTTTGATGG
CCACGCTGGCGAAGCCCCTGTGTCCATCATTAACAACGCCGTGTACACCA
AGGTGGACGGCATCGACGTGGAAATCTTCGAGAACAAGACCACACTGCCC
GTGAATGTGGCCTTCGAGCTGTGGGCCAAGCGGAACATTAAGCCCGTGCC
TGAGATCAAGATCCTGAACAACCTGGGCGTCGACATTGCCGCCAACACCG
TGATCTGGGACTACAAGAGAGAAGCCCCAGCTCACGTGTCCACCATCGGC
GTGTGTACCATGACCGATATCGCCAAGAAGCCCACCGAGAGCGCCTGTAG
CTCTCTGACCGTGCTGTTCGACGGCAGAGTGGAAGGCCAGGTGGACCTGT
TCAGAAACGCCAGAAACGGCGTGCTGATCACCGAGGGCTCTGTGAAGGGA
CTGACCCCTTCTAAGGGACCTGCTCAGGCCTCTGTGAATGGCGTGACACT
GATCGGCGAGAGCGTGAAAACCCAGTTCAACTACTTCAAGAAGGTCGACG
GGATCATCCAGCAGCTGCCCGAGACATACTTCACCCAGAGCCGCGACCTG
GAAGATTTCAAGCCTCGGAGCCAGATGGAAACCGACTTCCTGGAACTGGC
CATGGACGAGTTCATCCAGCGGTACAAGCTGGAAGGCTACGCCTTTGAGC
ACATCGTGTACGGCGATTTCAGCCACGGACAGCTCGGAGGACTGCACCTG
ATGATTGGCCTGGCCAAGAGAAGCCAGGACAGCCCTCTGAAGCTCGAGGA
CTTCATCCCCATGGACAGCACCGTGAAGAATTACTTCATCACAGACGCCC
AGACCGGCAGCTCTAAGTGCGTGTGTAGCGTGATCGACCTGCTGCTGGAC
GACTTTGTGGAAATCATCAAGAGCCAGGACCTGAGCGTGATCTCCAAGGT
GGTCAAAGTGACCATCGACTACGCCGAGATCAGCTTCATGCTGTGGTGCA
AGGACGGCCACGTGGAAACATTCTACCCCAAGCTGCAG

Hence, preferably the RNA construct is encoded by a DNA sequence substantially as set out in SEQ ID No: 261, or a fragment or variant thereof.

In an embodiment, the RNA sequence corresponding to the codon optimised DNA sequence of SEQ ID No: 261 is provided herein as SEQ ID No: 262, as follows:

[SEQ ID No: 262]
AGCCUGGAAAACGUGGCCUACAACGUGGUCAACAAGGGCCACUUUGAUGG
CCACGCUGGCGAAGCCCCUGUGUCCAUCAUUAACAACGCCGUGUACACCA
AGGUGGACGGCAUCGACGUGGAAAUCUUCGAGAACAAGACCACACUGCCC
GUGAAUGUGGCCUUCGAGCUGUGGGCCAAGCGGAACAUUAAGCCCGUGCC
UGAGAUCAAGAUCCUGAACAACCUGGGCGUCGACAUUGCCGCCAACACCG
UGAUCUGGGACUACAAGAGAGAAGCCCCAGCUCACGUGUCCACCAUCGGC
GUGUGUACCAUGACCGAUAUCGCCAAGAAGCCCACCGAGAGCGCCUGUAG
CUCUCUGACCGUGCUGUUCGACGGCAGAGUGGAAGGCCAGGUGGACCUGU
UCAGAAACGCCAGAAACGGCGUGCUGAUCACCGAGGGCUCUGUGAAGGGA
CUGACCCCUUCUAAGGGACCUGCUCAGGCCUCUGUGAAUGGCGUGACACU
GAUCGGCGAGAGCGUGAAAACCCAGUUCAACUACUUCAAGAAGGUCGACG
GGAUCAUCCAGCAGCUGCCCGAGACAUACUUCACCCAGAGCCGCGACCUG
GAAGAUUUCAAGCCUCGGAGCCAGAUGGAAACCGACUUCCUGGAACUGGC
CAUGGACGAGUUCAUCCAGCGGUACAAGCUGGAAGGCUACGCCUUUGAGC
ACAUCGUGUACGGCGAUUUCAGCCACGGACAGCUCGGAGGACUGCACCUG
AUGAUUGGCCUGGCCAAGAGAAGCCAGGACAGCCCUCUGAAGCUCGAGGA
CUUCAUCCCCAUGGACAGCACCGUGAAGAAUUACUUCAUCACAGACGCCC
AGACCGGCAGCUCUAAGUGCGUGUGUAGCGUGAUCGACCUGCUGCUGGAC
GACUUUGUGGAAAUCAUCAAGAGCCAGGACCUGAGCGUGAUCUCCAAGGU
GGUCAAAGUGACCAUCGACUACGCCGAGAUCAGCUUCAUGCUGUGGUGCA
AGGACGGCCACGUGGAAACAUUCUACCCCAAGCUGCAG

Hence, preferably the RNA construct comprises a sequence substantially as set out in SEQ ID No: 262, or a fragment or variant thereof.

In one embodiment, the at least one IIP is SARS CoV Orf9b (P59636; SARS ORF9b protein Severe acute respiratory syndrome coronavirus), or an orthologue thereof. One embodiment of the polypeptide sequence of SARS CoV Orf9b is represented herein as SEQ ID No: 263, as follows:

[SEQ ID No: 263]
MDPNQTNVVPPALHLVDPQIQLTITRMEDAMGQGQNSADPKVYPIILRLG
SQLSLSMARRNLDSLEARAFQSTPIVVQMTKLATTEELPDEFVVVTAK

Therefore, preferably the RNA construct comprises a nucleotide sequence which encodes an amino acid sequence substantially as set out in SEQ ID No: 263, or a variant or fragment thereof.

In one embodiment, the SARS CoV Orf9b polypeptide is encoded by the DNA nucleotide sequence of SEQ ID No: 264, as follows:

[SEQ ID No: 264]
ATGGACCCCAATCAAACCAACGTAGTGCCCCCCGCATTACATTTGGTGGA
CCCACAGATTCAACTGACAATAACCAGAATGGAGGACGCAATGGGGCAAG
GCCAAAACAGCGCCGACCCCAAGGTTTACCCAATAATACTGCGTCTTGGT
TCACAGCTCTCACTCAGCATGGCAAGGAGGAACTTAGATTCCCTCGAGGC
CAGGGCGTTCCAATCAACACCAATAGTGGTCCAGATGACCAAATTGGCTA
CTACCGAAGAGCTACCCGACGAGTTCGTGGTGGTGACGGCAAAA

Accordingly, preferably the SARS CoV Orf9b polypeptide is encoded by the DNA nucleotide sequence substantially as set out in SEQ ID NO: 264, or a variant or fragment thereof.

One embodiment of codon optimised nucleic acid (DNA) encoding the SARS CoV Orf9b polypeptide is provided herein as SEQ ID No: 265, as follows:

[SEQ ID No: 265]
ATGGACCCCAACCAGACCAATGTGGTGCCTCCTGCTCTGCACCTGGTGGA
CCCTCAGATCCAGCTGACCATCACCAGAATGGAAGATGCCATGGGCCAGG
GCCAGAACAGCGCCGATCCTAAGGTGTACCCCATCATCCTGAGACTGGGC
AGCCAGCTGAGCCTGAGCATGGCCAGAAGAAACCTGGACAGCCTGGAAGC
CAGAGCCTTCCAGAGCACACCTATCGTGGTGCAGATGACCAAGCTGGCCA
CCACCGAGGAACTGCCCGATGAGTTTGTGGTGGTCACCGCCAAA

Hence, preferably the RNA construct is encoded by a DNA sequence substantially as set out in SEQ ID No: 265, or a fragment or variant thereof.

In an embodiment, the RNA sequence corresponding to the codon optimised DNA sequence of SEQ ID No: 265 is provided herein as SEQ ID No: 266, as follows:

[SEQ ID No: 266]
AUGGACCCCAACCAGACCAAUGUGGUGCCUCCUGCUCUGCACCUGGUGGA
CCCUCAGAUCCAGCUGACCAUCACCAGAAUGGAAGAUGCCAUGGGCCAGG
GCCAGAACAGCGCCGAUCCUAAGGUGUACCCCAUCAUCCUGAGACUGGGC
AGCCAGCUGAGCCUGAGCAUGGCCAGAAGAAACCUGGACAGCCUGGAAGC
CAGAGCCUUCCAGAGCACACCUAUCGUGGUGCAGAUGACCAAGCUGGCCA
CCACCGAGGAACUGCCCGAUGAGUUUGUGGUGGUCACCGCCAAA

Hence, preferably the RNA construct comprises a sequence substantially as set out in SEQ ID No: 266, or a fragment or variant thereof.

In one embodiment, the at least one IIP is Rhinovirus 2Apro (P23008; Genome polyprotein Human rhinovirus 1A), or an orthologue thereof. One embodiment of the polypeptide sequence of Rhinovirus 2Apro is represented herein as SEQ ID No: 267, as follows:

[SEQ ID No: 267]
GPSDLYVHVGNLIYRNLHLFNSEMHDSILISYSSDLITYRTNTIGDDYIP
NCNCTEATYYCRHKNRYYPIKVTPHDWYEIQESEYYPKHIQYNLLIGEGP
CEPGDCGGKLLCRHGVIGIITAGGEGHVAFIDLRQFHCAEEQ

Therefore, preferably the RNA construct comprises a nucleotide sequence which encodes an amino acid sequence substantially as set out in SEQ ID No: 267, or a variant or fragment thereof.

In one embodiment, the Rhinovirus 2Apro polypeptide is encoded by the DNA nucleotide sequence of SEQ ID No: 268, as follows:

[SEQ ID No: 268]
GGGCCCAGTGATCTATATGTGCATGTAGGTAACTTAATATATAGAAACTT
ACATCTGTTCAATTCTGAAATGCATGATTCAATTTTGATTTCATACTCTT
CTGATTTAATCATATACCGCACAAACACTATAGGTGATGATTATATTCCC
AATTGTAACTGCACTGAGGCTACTTATTATTGTAGACACAAAAATAGGTA
TTACCCAATAAAAGTTACTCCACATGATTGGTATGAAATACAAGAGAGTG
AATATTACCCCAAACACATCCAATACAACCTATTAATTGGTGAAGGACCA
TGTGAACCTGGTGATTGTGGTGGAAAACTTCTTTGTAGACATGGTGTCAT
TGGCATAATCACAGCAGGTGGTGAAGGTCATGTAGCATTTATAGATCTTA
GACAATTTCACTGTGCTGAGGAACAA

Accordingly, preferably the Rhinovirus 2Apro polypeptide is encoded by the DNA nucleotide sequence substantially as set out in SEQ ID NO: 268, or a variant or fragment thereof.

One embodiment of codon optimised nucleic acid (DNA) encoding the Rhinovirus 2Apro polypeptide is provided herein as SEQ ID No: 269, as follows:

[SEQ ID No: 269]
GGCCCTAGCGATCTGTATGTGCACGTGGGCAACCTGATCTACCGGAACCT
GCACCTGTTCAACAGCGAGATGCACGACAGCATCCTGATCAGCTACAGCA
GCGACCTGATCATCTATCGGACCAACACCATCGGCGACGACTACATCCCC
AACTGCAACTGTACCGAGGCCACCTACTACTGCCGGCACAAGAACCGGTA
CTACCCCATCAAAGTGACCCCTCACGATTGGTACGAGATCCAAGAGAGCG
AGTACTACCCTAAGCACATCCAGTACAACCTGCTGATCGGCGAGGGACCT
TGCGAGCCTGGCGATTGTGGTGGAAAGCTGCTGTGTAGACACGGCGTGAT
CGGCATCATTACAGCCGGCGGAGAAGGACACGTGGCCTTTATCGACCTGC
GGCAGTTTCACTGCGCCGAGGAACAG

Hence, preferably the RNA construct is encoded by a DNA sequence substantially as set out in SEQ ID No: 269, or a fragment or variant thereof.

In an embodiment, the RNA sequence corresponding to the codon optimised DNA sequence of SEQ ID No: 269 is provided herein as SEQ ID No: 270, as follows:

[SEQ ID No: 270]
GGCCCUAGCGAUCUGUAUGUGCACGUGGGCAACCUGAUCUACCGGAACCU
GCACCUGUUCAACAGCGAGAUGCACGACAGCAUCCUGAUCAGCUACAGCA
GCGACCUGAUCAUCUAUCGGACCAACACCAUCGGCGACGACUACAUCCCC
AACUGCAACUGUACCGAGGCCACCUACUACUGCCGGCACAAGAACCGGUA
CUACCCCAUCAAAGUGACCCCUCACGAUUGGUACGAGAUCCAAGAGAGCG
AGUACUACCCUAAGCACAUCCAGUACAACCUGCUGAUCGGCGAGGGACCU
UGCGAGCCUGGCGAUUGUGGUGGAAAGCUGCUGUGUAGACACGGCGUGAU
CGGCAUCAUUACAGCCGGCGGAGAAGGACACGUGGCCUUUAUCGACCUGC
GGCAGUUUCACUGCGCCGAGGAACAG

Hence, preferably the RNA construct comprises a sequence substantially as set out in SEQ ID No: 270, or a fragment or variant thereof.

In one embodiment, the at least one IIP is Rhinovirus 3Cpro (P23008; Genome polyprotein Human rhinovirus 1A), or an orthologue thereof. One embodiment of the polypeptide sequence of Rhinovirus 3Cpro is represented herein as SEQ ID No: 271, as follows:

[SEQ ID No: 271]
GPEEEFGRSILKNNTCVITTGNGKFTGLGIHDRILIIPTHADPGREVQVN
GVHTKVLDSYDLYNRDGVKLEITVIQLDRNEKFRDIRKYIPETEDDYPEC
NLALSANQDEPTIIKVGDVVSYGNILLSGNQTARMLKYNYPTKSGYCGGV
LYKIGQILGIHVGGNGRDGFSAMLLRSYFTDTQ

Therefore, preferably the RNA construct comprises a nucleotide sequence which encodes an amino acid sequence substantially as set out in SEQ ID No: 271, or a variant or fragment thereof.

In one embodiment, the Rhinovirus 3Cpro polypeptide is encoded by the DNA nucleotide sequence of SEQ ID No: 272, as follows:

[SEQ ID No: 272]
GGTCCAGAAGAAGAATTTGGAAGGTCAATTCTCAAAAACAATACTTGTGT
GATTACTACAGGTAATGGAAAATTTACAGGTCTTGGTATACATGACAGAA
TTCTAATCATCCCAACACATGCTGATCCAGGTAGAGAGGTCCAAGTTAAT
GGTGTCCACACTAAGGTTCTAGACTCATATGATCTTTATAATAGAGATGG
AGTTAAACTTGAAATAACGGTCATACAATTAGATAGAAATGAAAAATTTA
GGGACATTAGAAAGTATATACCTGAAACAGAAGACGATTATCCAGAATGC
AATTTGGCACTTTCAGCTAATCAAGATGAACCAACTATAATTAAAGTAGG
AGATGTAGTGTCCTATGGCAATATTTTGCTTAGTGGAAATCAAACAGCCA
GAATGCTTAAATATAATTACCCCACAAAATCAGGGTATTGTGGAGGGGTA
CTATATAAAATTGGTCAAATTCTAGGTATTCATGTGGGTGGAAATGGAAG
GGATGGTTTTTCAGCTATGTTACTTAGATCATACTTTACAGATACTCAG

Accordingly, preferably the Rhinovirus 3Cpro polypeptide is encoded by the DNA nucleotide sequence substantially as set out in SEQ ID NO: 272, or a variant or fragment thereof.

One embodiment of codon optimised nucleic acid (DNA) encoding the Rhinovirus 3Cpro polypeptide is provided herein as SEQ ID No: 273, as follows:

[SEQ ID No: 273]
GGACCTGAGGAAGAGTTCGGCAGATCCATCCTGAAGAACAATACCTGCGT
GATCACCACCGGCAACGGCAAGTTTACAGGCCTGGGCATCCACGACCGGA
TCCTGATCATTCCCACACACGCCGATCCTGGCCGGGAAGTGCAAGTGAAT
GGCGTGCACACCAAGGTGCTGGACAGCTACGACCTGTACAACCGCGACGG
CGTGAAGCTGGAAATCACCGTGATTCAGCTGGACCGGAACGAGAAGTTCC
GGGACATCCGGAAGTACATCCCCGAGACAGAGGACGACTACCCCGAGTGT
AATCTGGCCCTGAGCGCCAACCAGGACGAGCCCACAATTATCAAAGTGGG
CGACGTGGTGTCCTACGGCAACATCCTGCTGTCCGGCAATCAGACCGCCA
GAATGCTGAAGTACAACTACCCCACCAAGAGCGGCTACTGTGGCGGCGTG
CTGTATAAGATCGGCCAGATCCTGGGAATTCACGTCGGCGGCAATGGCAG
AGATGGCTTCTCTGCTATGCTGCTGCGGAGCTACTTCACCGACACACAG

Hence, preferably the RNA construct is encoded by a DNA sequence substantially as set out in SEQ ID No: 273, or a fragment or variant thereof.

In an embodiment, the RNA sequence corresponding to the codon optimised DNA sequence of SEQ ID No: 273 is provided herein as SEQ ID No: 274, as follows:

[SEQ ID No: 274]
GGACCUGAGGAAGAGUUCGGCAGAUCCAUCCUGAAGAACAAUACCUGCGU
GAUCACCACCGGCAACGGCAAGUUUACAGGCCUGGGCAUCCACGACCGGA
UCCUGAUCAUUCCCACACACGCCGAUCCUGGCCGGGAAGUGCAAGUGAAU
GGCGUGCACACCAAGGUGCUGGACAGCUACGACCUGUACAACCGCGACGG
CGUGAAGCUGGAAAUCACCGUGAUUCAGCUGGACCGGAACGAGAAGUUCC
GGGACAUCCGGAAGUACAUCCCCGAGACAGAGGACGACUACCCCGAGUGU
AAUCUGGCCCUGAGCGCCAACCAGGACGAGCCCACAAUUAUCAAAGUGGG
CGACGUGGUGUCCUACGGCAACAUCCUGCUGUCCGGCAAUCAGACCGCCA
GAAUGCUGAAGUACAACUACCCCACCAAGAGCGGCUACUGUGGCGGCGUG
CUGUAUAAGAUCGGCCAGAUCCUGGGAAUUCACGUCGGCGGCAAUGGCAG
AGAUGGCUUCUCUGCUAUGCUGCUGCGGAGCUACUUCACCGACACACAG

Hence, preferably the RNA construct comprises a sequence substantially as set out in SEQ ID No: 274, or a fragment or variant thereof.

In one embodiment, the at least one IIP is Rotavirus VP3 (A2T3S5; Protein VP3 Rotavirus A (isolate RVA/Monkey/South Africa/SA11-H96/1958/G3P5B[2])), or an orthologue thereof. One embodiment of the polypeptide sequence of Rotavirus VP3 is represented herein as SEQ ID No: 275, as follows:

[SEQ ID No: 275]
MKVLALRHSVAQVYADTQVYVHDDTKDSYENAFLISNLTTHNILYLNYSI
KTLEILNKSGIAAIALQSLEELFTLIRCNFTYDYELDIIYLHDYSYYTNN
EIRTDQHWITKTNIEEYLLPGWKLTYVGYNGSETRGHYNFSFKCQNAATD
DDLIIEYIYSEALDFQNFMLKKIKERMTTSLPIARLSNRVFRDKLFPSLL
KEHKNVVNVGPRNESMFTFLNYPTIKQFSNGAYLVKDTIKLKQERWLGKR
ISQFDIGQYKNMLNVLTAIYYYYNLYKSKPIIYMIGSAPSYWIYDVRHYS
DFFFETWDPLDTPYSSIHHKELFFINDVKKLKDNSILYIDIRTDRGNADW
KKWRKTVEEQTINNLDIAYEYLRTGKAKVCCVKMTAMDLELPISAKLLHH
PTTEIRSEFYLLLDTWDLTNIRRFIPKGVLYSFINNIITENVFIQQPFKV
KVLNDSYIVALYALSNDFNNRSEVIKLINNQKQSLITVRINNTFKDEPKV
GFKNIYDWTFLPTDFDTKEAIITSYDGCLGLFGLSISLASKPTGNNHLFI
LSGTDKYYKLDQFANHTSISRRSHQIRFSESATSYSGYIFRDLSNNNFNL
IGTNIENSVSGHVYNALIYYRYNYSFDLKRWIYLHSIDKVDIEGGKYYEL
APIELIYACRSAKEFATLQDDLTVLRYSNEIENYINTVYSITYADDPNYF
IGIQFRNIPYKYDVKIPHLTFGVLHISDNMVPDVIDILKIMKNELFKMDI
TTSYTYMLSDGIYVANVSGVLSTYFKIYNVFYKNQITFGQSRMFIPHITL
SFNNMRTVRIETTKLQIKSTYLRKIKGDTVFDMVE

Therefore, preferably the RNA construct comprises a nucleotide sequence which encodes an amino acid sequence substantially as set out in SEQ ID No: 275, or a variant or fragment thereof.

In one embodiment, the Rotavirus VP3 polypeptide is encoded by the DNA nucleotide sequence of SEQ ID No: 276, as follows:

[SEQ ID No: 276]
ATGAAAGTACTAGCTTTAAGACACAGTGTGGCTCAAGTGTATGCAGACAC
TCAAGTCTACGTTCATGATGATACAAAAGATAGTTATGAAAACGCTTTTT
TAATCTCTAATCTTACGACCCATAATATTTTATACTTAAATTATAGCATT
AAAACATTAGAAATATTAAATAAGTCAGGAATAGCTGCAATTGCTTTACA
ATCACTTGAAGAATTATTCACATTAATAAGGTGTAATTTCACTTATGATT
ATGAACTTGATATAATATATTTACATGATTATTCATATTATACCAATAAT
GAAATTAGAACAGACCAACATTGGATAACAAAAACAAATATTGAAGAATA
TTTACTACCTGGATGGAAATTAACATATGTTGGTTATAATGGAAGTGAAA
CTAGAGGACATTATAACTTTTCATTTAAATGTCAAAACGCTGCAACAGAT
GATGATCTAATAATTGAATACATTTATTCAGAAGCGTTGGACTTCCAAAA
TTTTATGTTAAAAAAGATAAAGGAAAGAATGACTACATCGTTGCCTATAG
CTAGATTATCTAACAGAGTATTTAGGGATAAGTTATTCCCATCATTATTG
AAAGAACATAAGAATGTAGTGAACGTTGGTCCGCGTAATGAATCTATGTT
TACATTTTTAAATTATCCAACTATAAAACAATTTTCAAATGGTGCGTATT
TAGTAAAAGATACTATAAAATTAAAACAAGAACGATGGTTAGGTAAAAGG
ATATCTCAGTTTGATATTGGTCAGTATAAAAATATGCTGAATGTTCTTAC
AGCAATTTATTATTACTATAATTTATATAAAAGTAAACCAATTATATATA
TGATCGGATCTGCTCCATCTTATTGGATATATGACGTTAGGCATTATTCC
GATTTTTTCTTTGAAACTTGGGATCCATTGGACACACCATATTCATCAAT
CCATCACAAAGAATTATTTTTTATAAATGATGTGAAGAAACTGAAGGATA
ACTCAATATTGTATATTGATATAAGAACCGATAGGGGCAATGCTGATTGG
AAAAAATGGAGAAAGACAGTAGAAGAACAAACTATTAATAATTTGGACAT
AGCTTATGAATATTTACGAACGGGTAAAGCGAAGGTGTGTTGTGTTAAGA
TGACAGCTATGGATTTGGAACTGCCAATTTCAGCTAAATTACTGCACCAC
CCAACTACGGAAATAAGATCAGAATTTTATTTATTACTAGATACTTGGGA
TTTAACTAACATTAGGAGGTTCATTCCTAAAGGCGTGTTATATTCATTTA
TAAACAATATAATAACTGAAAATGTGTTTATTCAACAACCATTTAAAGTA
AAAGTACTGAATGATAGTTATATTGTAGCGTTATATGCATTATCAAATGA
TTTTAATAATAGATCAGAAGTAATTAAATTAATTAATAATCAGAAACAAT
CTCTAATAACTGTTAGAATAAATAATACGTTTAAGGATGAACCAAAAGTT
GGGTTCAAAAATATCTATGATTGGACCTTTCTTCCAACCGACTTTGATAC
CAAAGAAGCTATAATTACTTCATACGACGGTTGTTTAGGACTCTTTGGTT
TGTCTATATCGTTAGCATCAAAACCAACAGGGAATAATCATTTATTCATT
TTAAGTGGTACAGATAAGTATTATAAATTGGATCAATTTGCTAATCACAC
CAGTATATCGAGAAGATCACACCAAATTAGGTTTTCGGAATCTGCTACTT
CATATTCAGGTTATATATTTAGAGATTTGTCCAATAATAATTTTAATCTA
ATTGGTACTAATATAGAGAATTCAGTATCAGGTCATGTATATAATGCTTT
AATTTATTATAGATATAATTATTCATTTGATCTTAAACGCTGGATTTATT
TACATTCTATAGATAAAGTTGATATAGAAGGAGGAAAGTATTATGAACTC
GCACCAATAGAATTAATTTATGCATGTAGATCAGCAAAAGAATTTGCTAC
ATTGCAGGATGACTTAACTGTATTGAGATATTCAAACGAAATAGAGAATT
ATATTAATACAGTATATAGTATAACATACGCTGATGATCCGAATTACTTT
ATCGGAATACAATTTAGAAATATACCATATAAATATGATGTTAAAATACC
GCATTTAACCTTCGGAGTATTACATATTTCTGATAACATGGTGCCAGACG
TGATTGACATACTAAAGATAATGAAGAATGAATTATTTAAAATGGATATT
ACGACCAGTTATACATATATGTTATCAGATGGAATCTACGTAGCAAATGT
TAGTGGAGTATTATCTACATACTTTAAAATCTATAACGTATTTTATAAAA
ATCAAATAACTTTTGGCCAATCCAGAATGTTTATTCCGCACATAACATTA
AGCTTCAATAACATGAGAATGGTTGAG

Accordingly, preferably the Rotavirus VP3 polypeptide is encoded by the DNA nucleotide sequence substantially as set out in SEQ ID NO: 276, or a variant or fragment thereof.

One embodiment of codon optimised nucleic acid (DNA) encoding the Rotavirus VP3 polypeptide is provided herein as SEQ ID No: 277, as follows:

[SEQ ID No: 277]
ATGAAGGTGCTGGCCCTGAGACATTCTGTGGCCCAGGTGTACGCCGACAC
ACAGGTCTACGTGCACGACGACACCAAGGACAGCTACGAGAACGCCTTCC
TGATCAGCAACCTGACCACACACAACATCCTGTACCTGAACTACAGCATC
AAGACCCTCGAGATCCTGAACAAGAGCGGAATCGCCGCTATCGCCCTGCA
GAGCCTGGAAGAACTGTTCACCCTGATCCGGTGCAACTTCACCTACGACT
ACGAGCTGGACATCATCTACCTGCACGATTACAGCTACTACACCAACAAC
GAGATCCGGACCGACCAGCACTGGATCACCAAGACCAACATCGAGGAATA
CCTGCTGCCTGGCTGGAAGCTGACCTACGTGGGCTACAATGGCAGCGAGA
CACGGGGCCACTACAACTTCAGCTTCAAGTGCCAGAACGCCGCCACCGAC
GACGACCTGATCATCGAGTACATCTACAGCGAGGCCCTGGACTTCCAGAA
CTTCATGCTGAAGAAAATCAAAGAACGGATGACCACCAGCCTGCCTATCG
CCAGACTGAGCAACCGGGTGTTCCGGGACAAGCTGTTTCCCAGCCTGCTG
AAAGAACACAAGAACGTGGTCAACGTGGGCCCCAGAAACGAGAGCATGTT
CACCTTTCTGAACTACCCCACCATCAAGCAGTTCAGCAACGGCGCCTACC
TGGTCAAGGACACAATCAAGCTGAAGCAAGAGAGATGGCTGGGCAAGAGA
ATCAGCCAGTTCGACATCGGCCAGTACAAGAACATGCTGAACGTGCTGAC
CGCCATCTACTACTACTATAACCTGTACAAGAGCAAGCCGATCATCTACA
TGATTGGCAGCGCCCCTAGCTACTGGATCTACGACGTGCGGCACTACAGC
GACTTTTTCTTCGAAACCTGGGATCCCCTGGACACCCCTTACAGCAGCAT
CCACCACAAAGAGCTGTTCTTCATCAACGACGTGAAGAAGCTCAAGGACA
ACAGCATCCTCTACATCGACATCAGAACCGACCGGGGCAACGCCGACTGG
AAGAAATGGCGGAAAACCGTGGAAGAACAGACCATCAACAACCTGGATAT
CGCCTACGAGTACCTGCGGACCGGCAAGGCCAAAGTGTGCTGCGTGAAGA
TGACAGCCATGGACCTGGAACTGCCCATCAGCGCCAAACTGCTGCACCAT
CCTACCACCGAGATCAGAAGCGAGTTCTATCTGCTGCTGGACACCTGGGA
CCTGACCAATATCAGACGGTTCATCCCCAAGGGCGTGCTGTACTCCTTTA
TCAACAACATCATCACCGAGAACGTGTTCATCCAGCAGCCGTTCAAAGTG
AAAGTGCTGAACGACAGCTACATCGTGGCCCTGTACGCCCTGAGCAACGA
CTTCAACAATCGGAGCGAAGTGATCAAACTGATCAACAATCAGAAGCAGT
CCCTGATCACCGTGCGCATCAACAATACCTTCAAGGACGAGCCCAAAGTG
GGCTTCAAGAATATCTACGACTGGACCTTCCTGCCTACCGACTTCGACAC
CAAAGAGGCCATCATCACAAGCTACGACGGCTGCCTGGGCCTGTTTGGCC
TGTCTATTAGCCTGGCCAGCAAGCCCACCGGCAACAACCACCTGTTTATC
CTGAGCGGCACCGACAAGTACTACAAGCTGGATCAGTTCGCCAACCACAC
CAGCATCAGCAGAAGAAGCCACCAGATCCGGTTCAGCGAGAGCGCCACAA
GCTATAGCGGCTACATCTTCCGGGACCTGTCCAACAACAACTTCAACCTG
ATCGGCACGAACATCGAGAACAGCGTGTCCGGCCACGTGTACAACGCCCT
GATCTACTACCGGTACAACTACTCCTTCGACCTGAAGCGGTGGATCTATC
TGCACAGCATCGACAAGGTGGACATCGAAGGCGGCAAGTACTATGAGCTG
GCCCCTATCGAGCTGATCTACGCCTGCAGAAGCGCCAAAGAGTTCGCCAC
ACTGCAGGACGATCTGACCGTGCTGAGATACAGCAATGAGATCGAGAACT
ACATCAACACCGTGTACTCCATCACCTACGCCGACGATCCCAACTACTTC
ATCGGAATCCAGTTCCGCAACATCCCCTATAAGTACGACGTCAAGATCCC
TCACCTGACCTTCGGCGTGCTGCACATCAGCGACAACATGGTGCCCGACG
TGATCGACATCCTGAAGATCATGAAGAATGAGCTGTTCAAGATGGACATC
ACCACCAGCTACACCTACATGCTGAGCGACGGCATCTACGTGGCCAATGT
GTCTGGCGTGCTGAGCACCTACTTCAAGATCTACAACGTGTTCTACAAGA
ACCAGATCACCTTCGGCCAGAGCCGGATGTTCATCCCTCACATCACCCTG
AGCTTTAACAACATGCGGACCGTGCGGATCGAAACCACCAAGCTGCAGAT
CAAGAGCATCTACCTCCGGAAGATCAAGGGCGACACCGTGTTCGACATGG
TGGAA

Hence, preferably the RNA construct is encoded by a DNA sequence substantially as set out in SEQ ID No: 277, or a fragment or variant thereof.

In an embodiment, the RNA sequence corresponding to the codon optimised DNA sequence of SEQ ID No: 277 is provided herein as SEQ ID No: 278, as follows:

[SEQ ID No: 278]
AUGAAGGUGCUGGCCCUGAGACAUUCUGUGGCCCAGGUGUACGCCGACAC
ACAGGUCUACGUGCACGACGACACCAAGGACAGCUACGAGAACGCCUUCC
UGAUCAGCAACCUGACCACACACAACAUCCUGUACCUGAACUACAGCAUC
AAGACCCUCGAGAUCCUGAACAAGAGCGGAAUCGCCGCUAUCGCCCUGCA
GAGCCUGGAAGAACUGUUCACCCUGAUCCGGUGCAACUUCACCUACGACU
ACGAGCUGGACAUCAUCUACCUGCACGAUUACAGCUACUACACCAACAAC
GAGAUCCGGACCGACCAGCACUGGAUCACCAAGACCAACAUCGAGGAAUA
CCUGCUGCCUGGCUGGAAGCUGACCUACGUGGGCUACAAUGGCAGCGAGA
CACGGGGCCACUACAACUUCAGCUUCAAGUGCCAGAACGCCGCCACCGAC
GACGACCUGAUCAUCGAGUACAUCUACAGCGAGGCCCUGGACUUCCAGAA
CUUCAUGCUGAAGAAAAUCAAAGAACGGAUGACCACCAGCCUGCCUAUCG
CCAGACUGAGCAACCGGGUGUUCCGGGACAAGCUGUUUCCCAGCCUGCUG
AAAGAACACAAGAACGUGGUCAACGUGGGCCCCAGAAACGAGAGCAUGUU
CACCUUUCUGAACUACCCCACCAUCAAGCAGUUCAGCAACGGCGCCUACC
UGGUCAAGGACACAAUCAAGCUGAAGCAAGAGAGAUGGCUGGGCAAGAGA
AUCAGCCAGUUCGACAUCGGCCAGUACAAGAACAUGCUGAACGUGCUGAC
CGCCAUCUACUACUACUAUAACCUGUACAAGAGCAAGCCGAUCAUCUACA
UGAUUGGCAGCGCCCCUAGCUACUGGAUCUACGACGUGCGGCACUACAGC
GACUUUUUCUUCGAAACCUGGGAUCCCCUGGACACCCCUUACAGCAGCAU
CCACCACAAAGAGCUGUUCUUCAUCAACGACGUGAAGAAGCUCAAGGACA
ACAGCAUCCUCUACAUCGACAUCAGAACCGACCGGGGCAACGCCGACUGG
AAGAAAUGGCGGAAAACCGUGGAAGAACAGACCAUCAACAACCUGGAUAU
CGCCUACGAGUACCUGCGGACCGGCAAGGCCAAAGUGUGCUGCGUGAAGA
UGACAGCCAUGGACCUGGAACUGCCCAUCAGCGCCAAACUGCUGCACCAU
CCUACCACCGAGAUCAGAAGCGAGUUCUAUCUGCUGCUGGACACCUGGGA
CCUGACCAAUAUCAGACGGUUCAUCCCCAAGGGCGUGCUGUACUCCUUUA
UCAACAACAUCAUCACCGAGAACGUGUUCAUCCAGCAGCCGUUCAAAGUG
AAAGUGCUGAACGACAGCUACAUCGUGGCCCUGUACGCCCUGAGCAACGA
CUUCAACAAUCGGAGCGAAGUGAUCAAACUGAUCAACAAUCAGAAGCAGU
CCCUGAUCACCGUGCGCAUCAACAAUACCUUCAAGGACGAGCCCAAAGUG
GGCUUCAAGAAUAUCUACGACUGGACCUUCCUGCCUACCGACUUCGACAC
CAAAGAGGCCAUCAUCACAAGCUACGACGGCUGCCUGGGCCUGUUUGGCC
UGUCUAUUAGCCUGGCCAGCAAGCCCACCGGCAACAACCACCUGUUUAUC
CUGAGCGGCACCGACAAGUACUACAAGCUGGAUCAGUUCGCCAACCACAC
CAGCAUCAGCAGAAGAAGCCACCAGAUCCGGUUCAGCGAGAGCGCCACAA
GCUAUAGCGGCUACAUCUUCCGGGACCUGUCCAACAACAACUUCAACCUG
AUCGGCACGAACAUCGAGAACAGCGUGUCCGGCCACGUGUACAACGCCCU
GAUCUACUACCGGUACAACUACUCCUUCGACCUGAAGCGGUGGAUCUAUC
UGCACAGCAUCGACAAGGUGGACAUCGAAGGCGGCAAGUACUAUGAGCUG
GCCCCUAUCGAGCUGAUCUACGCCUGCAGAAGCGCCAAAGAGUUCGCCAC
ACUGCAGGACGAUCUGACCGUGCUGAGAUACAGCAAUGAGAUCGAGAACU
ACAUCAACACCGUGUACUCCAUCACCUACGCCGACGAUCCCAACUACUUC
AUCGGAAUCCAGUUCCGCAACAUCCCCUAUAAGUACGACGUCAAGAUCCC
UCACCUGACCUUCGGCGUGCUGCACAUCAGCGACAACAUGGUGCCCGACG
UGAUCGACAUCCUGAAGAUCAUGAAGAAUGAGCUGUUCAAGAUGGACAUC
ACCACCAGCUACACCUACAUGCUGAGCGACGGCAUCUACGUGGCCAAUGU
GUCUGGCGUGCUGAGCACCUACUUCAAGAUCUACAACGUGUUCUACAAGA
ACCAGAUCACCUUCGGCCAGAGCCGGAUGUUCAUCCCUCACAUCACCCUG
AGCUUUAACAACAUGCGGACCGUGCGGAUCGAAACCACCAAGCUGCAGAU
CAAGAGCAUCUACCUCCGGAAGAUCAAGGGCGACACCGUGUUCGACAUGG
UGGAA

Hence, preferably the RNA construct comprises a sequence substantially as set out in SEQ ID No: 278, or a fragment or variant thereof.

In one embodiment, the at least one IIP is GB Virus B NS3/4A (Q69422; Genome polyprotein Hepatitis GB virus B), or an orthologue thereof. One embodiment of the polypeptide sequence of GB Virus B NS3/4A is represented herein as SEQ ID No: 279, as follows:

[SEQ ID No: 279]
APFTLQCLSERGTLSAMAVVMTGIDPRTWTGTIFRLGSLATSYMGFVCDN
VLYTAHHGSKGRRLAHPTGSIHPITVDAANDQDIYQPPCGAGSLTRCSCG
ETKGYLVTRLGSLVEVNKSDDPYWCVCGALPMAVAKGSSGAPILCSSGHV
IGMFTAARNSGGSVSQIRVRPLVCAGYHPQYTAHATLDTKPTVPNEYSVQ
ILIAPTGSGKSTKLPLSYMQEKYEVLVLNPSVATTASMPKYMHATYGVNP
NCYFNGKCTNTGASLTYSTYGMYLTGACSRNYDVIICDECHATDATTVLG
IGKVLTEAPSKNVRLVVLATATPPGVIPTPHANITEIQLTDEGTIPFHGK
KIKEENLKKGRHLIFEATKKHCDELANELARKGITAVSYYRGCDISKIPE
GDCVVVATDALCTGYTGDFDSVYDCSLMVEGTCHVDLDPTFTMGVRVCGV
SAIVKGQRRGRTGRGRAGIYYYVDGSCTPSGMVPECNIVEAFDAAKAWYG
LSSTEAQTILDTYRTQPGLPAIGANLDEWADLFSMVNPEPSFVNTAKRTA
DNYVLLTAAQLQLCHQYGYAAPNDAPRWQGARLGKKPCGVLWRLDGADAC
PGPEPSEVTRYQMCFTEVNTSGTAALAVGVGVAMAYLAIDTFGATCVRRC
WSITSVPTGATVAPVVDEEEIVEEC

Therefore, preferably the RNA construct comprises a nucleotide sequence which encodes an amino acid sequence substantially as set out in SEQ ID No: 279, or a variant or fragment thereof.

In one embodiment, the GB Virus B NS3/4A polypeptide is encoded by the DNA nucleotide sequence of SEQ ID No: 280, as follows:

[SEQ ID No: 280]
GCACCTTTTACGCTGCAGTGTCTCTCTGAACGTGGCACGCTGTCAGCGAT
GGCAGTGGTCATGACTGGTATAGACCCCCGAACTTGGACTGGAACTATCT
TCAGATTAGGATCTCTGGCCACTAGCTACATGGGATTTGTTTGTGACAAC
GTGTTGTATACTGCTCACCATGGCAGCAAGGGGCGCCGGTTGGCTCATCC
CACAGGCTCCATACACCCAATAACCGTTGACGCGGCTAATGACCAGGACA
TCTATCAACCACCATGTGGAGCTGGGTCCCTTACTCGGTGCTCTTGCGGG
GAGACCAAGGGGTATCTGGTAACACGACTGGGGTCATTGGTTGAGGTCAA
CAAATCCGATGACCCTTATTGGTGTGTGTGCGGGGCCCTTCCCATGGCTG
TTGCCAAGGGTTCTTCAGGTGCCCCGATTCTGTGCTCCTCCGGGCATGTT
ATTGGGATGTTCACCGCTGCTAGAAATTCTGGCGGTTCAGTCAGCCAGAT
TAGGGTTAGGCCGTTGGTGTGTGCTGGATACCATCCCCAGTACACAGCAC
ATGCCACTCTTGATACAAAACCTACTGTGCCTAACGAGTATTCAGTGCAA
ATTTTAATTGCCCCCACTGGCAGCGGCAAGTCAACCAAATTACCACTTTC
TTACATGCAGGAGAAGTATGAGGTCTTGGTCCTAAATCCCAGTGTGGCTA
CAACAGCATCAATGCCAAAGTACATGCACGCGACGTACGGCGTGAATCCA
AATTGCTATTTTAATGGCAAATGTACCAACACAGGGGCTTCACTTACGTA
CAGCACATATGGCATGTACCTGACCGGAGCATGTTCCCGGAACTATGACG
TCATCATTTGTGACGAATGCCATGCTACCGATGCAACCACCGTGTTGGGC
ATTGGAAAGGTTCTAACCGAAGCTCCATCCAAAAATGTTAGGCTAGTGGT
TCTTGCCACGGCTACCCCCCCTGGAGTAATCCCTACACCACATGCCAACA
TAACTGAGATTCAATTAACCGATGAAGGCACTATCCCCTTTCATGGAAAA
AAGATTAAGGAGGAAAATCTGAAGAAAGGGAGACACCTTATCTTTGAGGC
TACCAAAAAACACTGTGATGAGCTTGCTAACGAGTTAGCTCGAAAGGGAA
TAACAGCTGTCTCTTACTATAGGGGATGTGACATCTCAAAAATCCCTGAG
GGCGACTGTGTAGTAGTTGCCACTGATGCCTTGTGTACAGGGTACACTGG
TGACTTTGATTCCGTGTATGACTGCAGCCTCATGGTAGAAGGCACATGCC
ATGTTGACCTTGACCCTACTTTCACCATGGGTGTTCGTGTGTGCGGGGTC
TCAGCAATAGTTAAAGGCCAGCGTAGGGGCCGCACAGGCCGTGGGAGAGC
TGGCATATACTACTATGTAGACGGGAGTTGTACCCCTTCGGGTATGGTTC
CTGAATGCAACATTGTTGAAGCCTTCGACGCAGCCAAGGCATGGTATGGT
TTGTCATCAACAGAAGCTCAAACTATTCTGGACACCTATCGCACCCAACC
TGGGTTACCTGCGATAGGAGCAAATTTGGACGAGTGGGCTGATCTCTTTT
CTATGGTCAACCCCGAACCTTCATTTGTCAATACTGCAAAAAGAACTGCT
GACAATTATGTTTTGTTGACTGCAGCCCAACTACAACTGTGTCATCAGTA
TGGCTATGCTGCTCCCAATGACGCACCACGGTGGCAGGGAGCCCGGCTTG
GGAAAAAACCTTGTGGGGTTCTGTGGCGCTTGGACGGCGCTGACGCCTGT
CCTGGCCCAGAGCCCAGCGAGGTGACCAGATACCAAATGTGCTTCACTGA
AGTCAATACTTCTGGGACAGCCGCACTCGCTGTTGGCGTTGGAGTGGCTA
TGGCTTATCTAGCCATTGACACTTTTGGCGCCACTTGTGTGCGGCGTTGC
TGGTCTATTACATCAGTCCCTACCGGTGCTACTGTCGCCCCAGTGGTTGA
CGAAGAAGAAATCGTGGAGGAGTGT

Accordingly, preferably the GB Virus B NS3/4A polypeptide is encoded by the DNA nucleotide sequence substantially as set out in SEQ ID NO: 280, or a variant or fragment thereof.

One embodiment of codon optimised nucleic acid (DNA) encoding the GB Virus B NS3/4A polypeptide is provided herein as SEQ ID No: 281, as follows:

[SEQ ID No: 281]
GCCCCTTTCACACTGCAATGCCTGAGCGAGAGAGGCACCCTGTCTGCCAT
GGCCGTGGTTATGACAGGCATCGACCCTAGAACCTGGACCGGCACCATCT
TCAGACTGGGAAGCCTGGCCACAAGCTACATGGGCTTCGTGTGCGACAAC
GTGCTGTACACAGCCCACCACGGCAGCAAAGGCAGAAGGCTGGCTCACCC
TACAGGCAGCATTCACCCCATCACAGTGGACGCCGCCAACGACCAGGATA
TCTACCAACCTCCTTGCGGCGCTGGCAGCCTGACCAGATGTTCTTGTGGC
GAGACAAAGGGCTACCTGGTCACCAGGCTGGGATCCCTGGTGGAAGTGAA
CAAGAGCGACGACCCCTATTGGTGCGTGTGTGGCGCACTGCCTATGGCTG
TGGCCAAAGGATCTTCTGGCGCCCCTATCCTGTGTAGCTCTGGCCACGTG
ATCGGCATGTTTACCGCCGCCAGAAATAGCGGCGGCAGCGTGTCACAGAT
TAGAGTGCGGCCTCTTGTGTGCGCCGGCTATCACCCTCAGTATACAGCCC
ACGCCACACTGGACACCAAGCCTACCGTGCCTAACGAGTACAGCGTGCAG
ATCCTGATCGCCCCAACAGGCAGCGGCAAGAGCACAAAACTGCCCCTGAG
CTACATGCAAGAGAAGTACGAGGTGCTGGTGCTGAACCCTAGCGTGGCCA
CAACAGCCAGCATGCCCAAGTACATGCACGCCACCTATGGCGTGAACCCC
AACTGCTACTTCAACGGCAAGTGCACCAATACCGGCGCCAGCCTGACATA
CAGCACCTACGGCATGTATCTGACCGGCGCCTGCAGCAGAAACTACGACG
TGATCATCTGCGACGAGTGCCACGCCACCGATGCCACAACTGTGCTCGGA
ATCGGCAAGGTGCTGACAGAGGCCCCTAGCAAGAATGTGCGACTGGTGGT
GCTGGCCACTGCTACACCACCTGGCGTTATCCCTACACCTCACGCCAACA
TCACCGAGATCCAGCTGACCGACGAGGGCACAATCCCATTCCACGGCAAG
AAGATCAAAGAGGAAAACCTGAAGAAGGGCCGCCACCTGATCTTCGAGGC
CACCAAGAAACACTGTGACGAGCTGGCCAACGAACTGGCCAGAAAGGGCA
TCACCGCCGTGTCCTACTACAGAGGCTGCGACATCAGCAAGATCCCCGAG
GGCGATTGTGTGGTGGTGGCTACAGATGCCCTGTGTACCGGCTACACCGG
CGACTTCGATAGCGTGTACGACTGCAGCCTGATGGTGGAAGGCACCTGTC
ATGTGGATCTGGACCCCACCTTTACCATGGGCGTCAGAGTGTGCGGAGTG
TCCGCCATCGTGAAGGGCCAGAGAAGAGGCAGAACTGGCAGAGGCAGAGC
CGGCATCTACTACTATGTGGACGGCAGCTGTACCCCTAGCGGCATGGTGC
CTGAGTGCAACATCGTGGAAGCCTTCGATGCCGCCAAGGCTTGGTACGGC
CTGTCTAGCACAGAGGCTCAGACCATCCTGGACACCTACAGAACCCAGCC
AGGACTGCCTGCCATCGGCGCCAATCTTGATGAATGGGCCGACCTGTTCA
GCATGGTCAACCCCGAGCCTAGCTTCGTGAACACCGCCAAGAGAACCGCC
GACAACTACGTGCTGCTGACAGCCGCTCAGCTCCAGCTGTGTCACCAGTA
CGGATACGCCGCTCCTAACGACGCCCCTAGATGGCAAGGCGCTAGACTGG
GCAAGAAACCATGTGGCGTTCTGTGGCGACTGGATGGCGCTGATGCTTGT
CCTGGACCTGAGCCTAGCGAAGTGACCAGATACCAGATGTGCTTCACCGA
AGTGAATACCAGCGGCACAGCTGCCCTGGCCGTTGGTGTTGGAGTGGCCA
TGGCTTACCTGGCCATCGATACCTTTGGCGCCACATGCGTGCGGAGATGC
TGGTCTATCACCAGCGTTCCAACAGGCGCTACAGTGGCCCCTGTGGTGGA
TGAGGAAGAGATTGTGGAAGAGTGC

Hence, preferably the RNA construct is encoded by a DNA sequence substantially as set out in SEQ ID No: 281, or a fragment or variant thereof.

In an embodiment, the RNA sequence corresponding to the codon optimised DNA sequence of SEQ ID No: 281 is provided herein as SEQ ID No: 282, as follows:

[SEQ ID No: 282]
GCCCCUUUCACACUGCAAUGCCUGAGCGAGAGAGGCACCCUGUCUGCCAU
GGCCGUGGUUAUGACAGGCAUCGACCCUAGAACCUGGACCGGCACCAUCU
UCAGACUGGGAAGCCUGGCCACAAGCUACAUGGGCUUCGUGUGCGACAAC
GUGCUGUACACAGCCCACCACGGCAGCAAAGGCAGAAGGCUGGCUCACCC
UACAGGCAGCAUUCACCCCAUCACAGUGGACGCCGCCAACGACCAGGAUA
UCUACCAACCUCCUUGCGGCGCUGGCAGCCUGACCAGAUGUUCUUGUGGC
GAGACAAAGGGCUACCUGGUCACCAGGCUGGGAUCCCUGGUGGAAGUGAA
CAAGAGCGACGACCCCUAUUGGUGCGUGUGUGGCGCACUGCCUAUGGCUG
UGGCCAAAGGAUCUUCUGGCGCCCCUAUCCUGUGUAGCUCUGGCCACGUG
AUCGGCAUGUUUACCGCCGCCAGAAAUAGCGGCGGCAGCGUGUCACAGAU
UAGAGUGCGGCCUCUUGUGUGCGCCGGCUAUCACCCUCAGUAUACAGCCC
ACGCCACACUGGACACCAAGCCUACCGUGCCUAACGAGUACAGCGUGCAG
AUCCUGAUCGCCCCAACAGGCAGCGGCAAGAGCACAAAACUGCCCCUGAG
CUACAUGCAAGAGAAGUACGAGGUGCUGGUGCUGAACCCUAGCGUGGCCA
CAACAGCCAGCAUGCCCAAGUACAUGCACGCCACCUAUGGCGUGAACCCC
AACUGCUACUUCAACGGCAAGUGCACCAAUACCGGCGCCAGCCUGACAUA
CAGCACCUACGGCAUGUAUCUGACCGGCGCCUGCAGCAGAAACUACGACG
UGAUCAUCUGCGACGAGUGCCACGCCACCGAUGCCACAACUGUGCUCGGA
AUCGGCAAGGUGCUGACAGAGGCCCCUAGCAAGAAUGUGCGACUGGUGGU
GCUGGCCACUGCUACACCACCUGGCGUUAUCCCUACACCUCACGCCAACA
UCACCGAGAUCCAGCUGACCGACGAGGGCACAAUCCCAUUCCACGGCAAG
AAGAUCAAAGAGGAAAACCUGAAGAAGGGCCGCCACCUGAUCUUCGAGGC
CACCAAGAAACACUGUGACGAGCUGGCCAACGAACUGGCCAGAAAGGGCA
UCACCGCCGUGUCCUACUACAGAGGCUGCGACAUCAGCAAGAUCCCCGAG
GGCGAUUGUGUGGUGGUGGCUACAGAUGCCCUGUGUACCGGCUACACCGG
CGACUUCGAUAGCGUGUACGACUGCAGCCUGAUGGUGGAAGGCACCUGUC
AUGUGGAUCUGGACCCCACCUUUACCAUGGGCGUCAGAGUGUGCGGAGUG
UCCGCCAUCGUGAAGGGCCAGAGAAGAGGCAGAACUGGCAGAGGCAGAGC
CGGCAUCUACUACUAUGUGGACGGCAGCUGUACCCCUAGCGGCAUGGUGC
CUGAGUGCAACAUCGUGGAAGCCUUCGAUGCCGCCAAGGCUUGGUACGGC
CUGUCUAGCACAGAGGCUCAGACCAUCCUGGACACCUACAGAACCCAGCC
AGGACUGCCUGCCAUCGGCGCCAAUCUUGAUGAAUGGGCCGACCUGUUCA
GCAUGGUCAACCCCGAGCCUAGCUUCGUGAACACCGCCAAGAGAACCGCC
GACAACUACGUGCUGCUGACAGCCGCUCAGCUCCAGCUGUGUCACCAGUA
CGGAUACGCCGCUCCUAACGACGCCCCUAGAUGGCAAGGCGCUAGACUGG
GCAAGAAACCAUGUGGCGUUCUGUGGCGACUGGAUGGCGCUGAUGCUUGU
CCUGGACCUGAGCCUAGCGAAGUGACCAGAUACCAGAUGUGCUUCACCGA
AGUGAAUACCAGCGGCACAGCUGCCCUGGCCGUUGGUGUUGGAGUGGCCA
UGGCUUACCUGGCCAUCGAUACCUUUGGCGCCACAUGCGUGCGGAGAUGC
UGGUCUAUCACCAGCGUUCCAACAGGCGCUACAGUGGCCCCUGUGGUGGA
UGAGGAAGAGAUUGUGGAAGAGUGC

Hence, preferably the RNA construct comprises a sequence substantially as set out in SEQ ID No: 282, or a fragment or variant thereof.

In one embodiment, the at least one IIP is HAV 3Cpro (P08617; Genome polyprotein Human hepatitis A virus genotype IB (isolate HM175)), or an orthologue thereof. One embodiment of the polypeptide sequence of HAV 3Cpro is represented herein as SEQ ID No: 283, as follows:

[SEQ ID No: 283]
STLEIAGLVRKNLVQFGVGEKNGCVRWVMNALGVKDDWLLVPSHAYKFEK
DYEMMEFYFNRGGTYYSISAGNVVIQSLDVGFQDVVLMKVPTIPKFRDIT
QHFIKKGDVPRALNRLATLVTTVNGTPMLISEGPLKMEEKATYVHKKNDG
TTVDLTVDQAWRGKGEGLPGMCGGALVSSNQSIQNAILGIHVAGGNSILV
AKLVTQEMFQNIDKKIESQ

Therefore, preferably the RNA construct comprises a nucleotide sequence which encodes an amino acid sequence substantially as set out in SEQ ID No: 283, or a variant or fragment thereof.

In one embodiment, the HAV 3Cpro polypeptide is encoded by the DNA nucleotide sequence of SEQ ID No: 284, as follows:

[SEQ ID No: 284]
TCAACTTTGGAAATAGCAGGACTGGTTAGGAAGAACTTGGTTCAGTTTGG
AGTTGGAGAGAAGAATGGATGTGTGAGATGGGTTATGAATGCCTTGGGAG
TGAAAGATGATTGGCTGCTTGTGCCTTCCCATGCTTATAAATTTGAGAAA
GATTATGAAATGATGGAGTTTTATTTTAATAGAGGTGGAACTTACTATTC
AATTTCAGCTGGTAATGTTGTTATTCAATCTTTGGATGTGGGATTCCAGG
ATGTTGTTCTGATGAAGGTTCCTACAATTCCTAAGTTTAGAGATATTACT
CAGCATTTTATTAAGAAAGGGGATGTGCCTAGAGCTTTGAATCGCCTGGC
AACATTAGTGACAACTGTAAATGGAACCCCTATGTTAATTTCTGAGGGCC
CACTAAAGATGGAAGAGAAAGCTACTTATGTTCATAAGAAAAATGATGGT
ACAACAGTTGATTTAACTGTGGATCAGGCATGGAGAGGAAAAGGCGAAGG
TCTTCCTGGAATGTGTGGTGGGGCCTTGGTTTCATCGAATCAATCTATAC
AGAATGCAATCTTGGGCATCCATGTTGCTGGAGGAAATTCAATTCTTGTT
GCAAAATTGGTTACTCAAGAAATGTTCCAAAATATTGATAAGAAAATTGA
AAGTCAG

Accordingly, preferably the HAV 3Cpro polypeptide is encoded by the DNA nucleotide sequence substantially as set out in SEQ ID NO: 284, or a variant or fragment thereof.

One embodiment of codon optimised nucleic acid (DNA) encoding the HAV 3Cpro polypeptide is provided herein as SEQ ID No: 285, as follows:

[SEQ ID No: 285]
AGCACACTGGAAATCGCCGGACTCGTGCGGAAGAACCTGGTGCAGTTTGGCGTGGGCGAGAAGAACGGCTGTGTCAGA
TGGGTCATGAACGCCCTGGGCGTGAAGGACGATTGGCTGCTGGTTCCTAGCCACGCCTACAAGTTCGAGAAGGACTAC
GAGATGATGGAATTCTACTTCAACAGAGGCGGCACCTACTACAGCATCAGCGCCGGCAATGTGGTCATCCAGTCTCTG
GATGTGGGCTTCCAGGACGTGGTGCTGATGAAGGTGCCAACAATCCCCAAGTTCCGGGACATCACCCAGCACTTCATC
AAGAAAGGCGACGTGCCCAGGGCTCTGAACAGACTGGCTACCCTGGTCACCACCGTGAACGGCACACCCATGCTGATC
TCTGAGGGCCCACTGAAGATGGAAGAGAAGGCCACCTACGTGCACAAGAAGAACGACGGCACCACAGTGGACCTGACC
GTGGATCAAGCTTGGAGAGGCAAAGGCGAGGGCCTGCCTGGAATGTGTGGCGGAGCACTGGTGTCCAGCAACCAGAGC
ATCCAGAATGCCATCCTGGGCATCCATGTGGCTGGCGGCAATTCTATCCTGGTGGCCAAGCTGGTCACCCAAGAGATG
TTCCAGAACATCGACAAGAAGATCGAGAGCCAG

Hence, preferably the RNA construct is encoded by a DNA sequence substantially as set out in SEQ ID No: 285, or a fragment or variant thereof.

In an embodiment, the RNA sequence corresponding to the codon optimised DNA sequence of SEQ ID No: 285 is provided herein as SEQ ID No: 286, as follows:

[SEQ ID No: 286]
AGCACACUGGAAAUCGCCGGACUCGUGCGGAAGAACCUGGUGCAGUUUGGCGUGGGCGAGAAGAACGGCUGUGUCAGA
UGGGUCAUGAACGCCCUGGGCGUGAAGGACGAUUGGCUGCUGGUUCCUAGCCACGCCUACAAGUUCGAGAAGGACUAC
GAGAUGAUGGAAUUCUACUUCAACAGAGGCGGCACCUACUACAGCAUCAGCGCCGGCAAUGUGGUCAUCCAGUCUCUG
GAUGUGGGCUUCCAGGACGUGGUGCUGAUGAAGGUGCCAACAAUCCCCAAGUUCCGGGACAUCACCCAGCACUUCAUC
AAGAAAGGCGACGUGCCCAGGGCUCUGAACAGACUGGCUACCCUGGUCACCACCGUGAACGGCACACCCAUGCUGAUC
UCUGAGGGCCCACUGAAGAUGGAAGAGAAGGCCACCUACGUGCACAAGAAGAACGACGGCACCACAGUGGACCUGACC
GUGGAUCAAGCUUGGAGAGGCAAAGGCGAGGGCCUGCCUGGAAUGUGUGGCGGAGCACUGGUGUCCAGCAACCAGAGC
AUCCAGAAUGCCAUCCUGGGCAUCCAUGUGGCUGGCGGCAAUUCUAUCCUGGUGGCCAAGCUGGUCACCCAAGAGAUG
UUCCAGAACAUCGACAAGAAGAUCGAGAGCCAG

Hence, preferably the RNA construct comprises a sequence substantially as set out in SEQ ID No: 286, or a fragment or variant thereof.

In one embodiment, the at least one IIP is Human metapneumovirus M2-2 (Q6WB96; Protein M2-2 Human metapneumovirus (strain CAN97-83)), or an orthologue thereof. One embodiment of the polypeptide sequence of Human metapneumovirus M2-2 is represented herein as SEQ ID No: 287, as follows:

[SEQ ID No: 287]
MTLHMPCKTVKALIKCSEHGPVFITIEVDEMIWTQKELKEALSDGIVKS
HTNIYNCYLENIEITYVKAYLS

Therefore, preferably the RNA construct comprises a nucleotide sequence which encodes an amino acid sequence substantially as set out in SEQ ID No: 287, or a variant or fragment thereof.

In one embodiment, the Human metapneumovirus M2-2 polypeptide is encoded by the DNA nucleotide sequence of SEQ ID No: 288, as follows:

[SEQ ID No: 288]
ATGACTCTTCATATGCCCTGCAAGACAGTGAAAGCATTAATCAAGTGCAGTGAGCATGGTCCTGTTTTCATTACTATA
GAGGTTGATGAAATGATATGGACTCAAAAAGAATTAAAAGAAGCTTTGTCCGATGGGATAGTGAAGTCTCACACCAAC
ATTTACAATTGTTATTTAGAAAACATAGAAATTATATATGTCAAGGCTTACTTAAGT

Accordingly, preferably the Human metapneumovirus M2-2 polypeptide is encoded by the DNA nucleotide sequence substantially as set out in SEQ ID NO: 288, or a variant or fragment thereof.

One embodiment of codon optimised nucleic acid (DNA) encoding the Human metapneumovirus M2-2 polypeptide is provided herein as SEQ ID No: 289, as follows:

[SEQ ID No: 289]
ATGACCCTGCACATGCCCTGCAAGACAGTGAAGGCCCTGATCAAGTGTAGCGAGCACGGCCCCGTGTTCATCACCATT
GAGGTGGACGAGATGATCTGGACCCAGAAAGAGCTGAAAGAGGCCCTGAGCGACGGCATCGTGAAGTCCCACACCAAC
ATCTACAACTGCTACCTCGAGAACATCGAGATCATCTACGTGAAGGCCTACCTGAGC

Hence, preferably the RNA construct is encoded by a DNA sequence substantially as set out in SEQ ID No: 289, or a fragment or variant thereof.

In an embodiment, the RNA sequence corresponding to the codon optimised DNA sequence of SEQ ID No: 289 is provided herein as SEQ ID No: 290, as follows:

[SEQ ID No: 290]
AUGACCCUGCACAUGCCCUGCAAGACAGUGAAGGCCCUGAUCAAGUGUAGCGAGCACGGCCCCGUGUUCAUCACCAUU
GAGGUGGACGAGAUGAUCUGGACCCAGAAAGAGCUGAAAGAGGCCCUGAGCGACGGCAUCGUGAAGUCCCACACCAAC
AUCUACAACUGCUACCUCGAGAACAUCGAGAUCAUCUACGUGAAGGCCUACCUGAGC

Hence, preferably the RNA construct comprises a sequence substantially as set out in SEQ ID No: 290, or a fragment or variant thereof.

In one embodiment, the at least one IIP is Hepatitis E methyltransferase (Q9WC28; Non-structural polyprotein pORF1 Methyltransferase Hepatitis E virus genotype 1), or an orthologue thereof. One embodiment of the polypeptide sequence of Hepatitis E methyltransferase is represented herein as SEQ ID No: 291, as follows:

[SEQ ID No: 291]
EVFWNQPIQRVIHNELELYCRARSGRCLEIGAHPRSINDNPNVVHRCFLRPVGRDVQRWYTAPTRGPAANCRRSALRG
LPAADRTYCFDGFSGCSCPAETGIALYSLHDMSPSDVAEAMFRHGMTRLYAALHLPPEVLLPPGTYRTASYLLIHDGR
RVVVTYEGDTSAGYNHDVSNLRSWI

Therefore, preferably the RNA construct comprises a nucleotide sequence which encodes an amino acid sequence substantially as set out in SEQ ID No: 291, or a variant or fragment thereof.

In one embodiment, the Hepatitis E methyltransferase polypeptide is encoded by the DNA nucleotide sequence of SEQ ID No: 292, as follows:

[SEQ ID No: 292]
GAGGTTTTCTGGAATCAACCCATCCAGCGTGTCATTCATAACGAGCTGGAGCTTTACTGCCGCGCTCGCTCCGGCCGC
TGTCTTGAAATTGGCGCCCATCCCCGCTCAATAAATGATAATCCTAATGTGGTCCACCGCTGCTTCCTCCGCCCTGTT
GGGCGTGATGTTCAGCGCTGGTATACTGCTCCCACTCGCGGGCCGGCTGCTAATTGCCGCCGTTCCGCGTTGCGTGGG
CTTCCCGCTGCTGACCGCACATACTGCTTCGACGGGTTTTCTGGCTGTAGCTGCCCCGCCGAGACGGGTATCGCCCTT
TACTCCCTCCATGATATGTCACCATCTGATGTTGCCGAGGCCATGTTCCGCCATGGTATGACGCGGCTTTATGCTGCC
CTCCATCTTCCGCCTGAGGTCTTGCTGCCCCCTGGCACATATCGCACCGCATCGTATTTGCTGATTCATGACGGCAGG
CGCGTTGTGGTGACGTATGAGGGTGATACTAGTGCTGGTTACAACCACGATGTCTCCAACTTGCGCTCCTGGATT

Accordingly, preferably the Hepatitis E methyltransferase polypeptide is encoded by the DNA nucleotide sequence substantially as set out in SEQ ID NO: 292, or a variant or fragment thereof.

One embodiment of codon optimised nucleic acid (DNA) encoding the Hepatitis E methyltransferase polypeptide is provided herein as SEQ ID No: 293, as follows:

[SEQ ID No: 293]
GAGGTGTTCTGGAACCAGCCTATCCAGAGAGTGATCCACAACGAGCTGGAACTGTACTGCAGAGCCAGATCCGGCCGG
TGTCTGGAAATTGGAGCCCATCCTCGGAGCATCAACGACAACCCCAACGTGGTGCACAGATGCTTTCTGAGGCCCGTG
GGCAGAGATGTGCAGCGGTGGTATACAGCCCCTACAAGAGGACCTGCCGCCAACTGTAGAAGAAGCGCCCTGAGAGGA
CTGCCTGCCGCCGATAGAACCTACTGCTTCGATGGCTTCAGCGGCTGCAGCTGTCCTGCCGAAACTGGAATCGCCCTG
TACAGCCTGCACGACATGAGCCCATCTGATGTGGCCGAGGCCATGTTCAGACACGGCATGACCAGACTGTACGCCGCT
CTGCATCTGCCTCCAGAAGTTCTGCTGCCTCCTGGCACCTACAGAACCGCCAGCTATCTGCTGATCCACGATGGGAGA
AGAGTGGTGGTCACCTACGAGGGCGATACAAGCGCCGGCTACAACCACGACGTGTCCAACCTGAGAAGCTGGATC

Hence, preferably the RNA construct is encoded by a DNA sequence substantially as set out in SEQ ID No: 293, or a fragment or variant thereof.

In an embodiment, the RNA sequence corresponding to the codon optimised DNA sequence of SEQ ID No: 293 is provided herein as SEQ ID No: 294, as follows:

[SEQ ID No: 294]
GAGGUGUUCUGGAACCAGCCUAUCCAGAGAGUGAUCCACAACGAGCUGGAACUGUACUGCAGAGCCAGAUCCGGCCGG
UGUCUGGAAAUUGGAGCCCAUCCUCGGAGCAUCAACGACAACCCCAACGUGGUGCACAGAUGCUUUCUGAGGCCCGUG
GGCAGAGAUGUGCAGCGGUGGUAUACAGCCCCUACAAGAGGACCUGCCGCCAACUGUAGAAGAAGCGCCCUGAGAGGA
CUGCCUGCCGCCGAUAGAACCUACUGCUUCGAUGGCUUCAGCGGCUGCAGCUGUCCUGCCGAAACUGGAAUCGCCCUG
UACAGCCUGCACGACAUGAGCCCAUCUGAUGUGGCCGAGGCCAUGUUCAGACACGGCAUGACCAGACUGUACGCCGCU
CUGCAUCUGCCUCCAGAAGUUCUGCUGCCUCCUGGCACCUACAGAACCGCCAGCUAUCUGCUGAUCCACGAUGGGAGA
AGAGUGGUGGUCACCUACGAGGGCGAUACAAGCGCCGGCUACAACCACGACGUGUCCAACCUGAGAAGCUGGAUC

Hence, preferably the RNA construct comprises a sequence substantially as set out in SEQ ID No: 294, or a fragment or variant thereof.

In one embodiment, the at least one IIP is PRRV Npro (P24381; Serine/threonine-protein kinase US3 homolog Suid herpesvirus 2 (strain NIA-3) Pseudorabies Virus), or an orthologue thereof. One embodiment of the polypeptide sequence of PRRV Npro is represented herein as SEQ ID No: 295, as follows:

[SEQ ID No: 295]
MLAMWRWVTKRSRLRRGHAHLGGNKGVRGICSLYLAGLSRGLSRVHAQRSHAATMADAGIPDEILYSDISDDEIIIDG
DGDGDSSGDEDDDDGGLTRQAASRIATDLGFEVLQPLQSGSEGRVFVARRPGEADTVVLKVGQKPSTLMEGMLLKRLA
HDNVMSLKQMLARGPVTCLVLPHFRCDLYSYLTMRDGPLDMRDAGRVIRSVLRGLAYLHGMRIMHRDVKAENIFLEDV
DTVCLGDLGAARCNVAAPNFYGLAGTIETNAPEVLARDRYDTKVDVWGAGVVLFETLAYPKTIAGGDEPAINGEMHLI
DLIRALGVHPEEFPPDTRLRSEFVRYAGTHRQPYTQYARVARLGLPETGAFLIYKMLTFDPVRRPSADEILNFGMWTV

Therefore, preferably the RNA construct comprises a nucleotide sequence which encodes an amino acid sequence substantially as set out in SEQ ID No: 295, or a variant or fragment thereof.

In one embodiment, the PRRV Npro polypeptide is encoded by the DNA nucleotide sequence of SEQ ID No: 296, as follows:

[SEQ ID No: 296]
ATGCTGGCGATGTGGAGATGGGTCACCAAGAGGTCGCGGCTCCGCCGAGGCCACGCCCATCTTGGGGGAAATAAAGGA
GTCCGGGGAATTTGTTCCTTATACCTTGCCGGGCTCAGCAGGGGGTTGTCGCGCGTCCACGCCCAGCGCTCGCACGCA
GCAACAATGGCCGACGCCGGAATCCCCGACGAGATCCTGTACTCGGACATCAGCGACGACGAGATCATCATCGACGGC
GACGGCGACGGCGACAGCAGCGGGGACGAGGACGACGATGACGGGGGGCTGACGCGGCAGGCCGCGTCGCGCATCGCC
ACGGACCTGGGCTTCGAGGTGCTGCAGCCCCTGCAGTCGGGCTCGGAGGGCCGCGTCTTCGTGGCCCGCCGGCCCGGC
GAGGCGGACACGGTGGTGCTGAAGGTGGGCCAGAAGCCCTCGACGCTGATGGAGGGCATGCTGCTGAAGCGCCTGGCC
CACGATAACGTCATGAGCCTGAAGCAGATGCTCGCCCGGGGCCCGGTGACGTGCCTGGTCCTGCCGCACTTTCGGTGC
GATCTGTACAGCTACCTGACCATGCGGGACGGGCCGCTGGACATGCGCGACGCCGGGCGCGTGATCCGGTCCGTGCTC
CGCGGGCTCGCCTACCTGCACGGGATGCGCATCATGCACCGCGACGTCAAGGCGGAGAACATCTTCCTCGAGGACGTG
GACACGGTGTGCCTGGGGGACCTCGGGGCCGCGCGCTGCAACGTGGCGGCGCCCAACTTTTACGGGCTCGCCGGGACC
ATCGAGACCAACGCCCCCGAGGTGCTCGCGCGCGACCGCTACGACACCAAGGTCGACGTCTGGGGCGCGGGGGTGGTG
CTCTTCGAGACGCTGGCCTACCCCAAGACGATCGCCGGCGGGGACGAGCCCGCGATCAACGGGGAGATGCACCTGATC
GACCTCATCCGCGCCCTCGGGGTGCACCCCGAGGAGTTCCCGCCCGACACGCGCCTCCGGAGCGAGTTCGTCCGGTAC
GCCGGGACCCACCGCCAGCCGTACACGCAGTACGCGCGCGTGGCTCGCCTCGGGCTGCCCGAGACGGGGGCTTTCCTG
ATTTACAAGATGTTGACGTTTGATCCCGTCCGCCGCCCTTCCGCTGATGAGATACTCAACTTTGGAATGTGGACCGTA

Accordingly, preferably the PRRV Npro polypeptide is encoded by the DNA nucleotide sequence substantially as set out in SEQ ID NO: 296, or a variant or fragment thereof.

One embodiment of codon optimised nucleic acid (DNA) encoding the PRRV Npro polypeptide is provided herein as SEQ ID No: 297, as follows:

[SEQ ID No: 297]
ATGCTGGCCATGTGGCGCTGGGTCACCAAGAGAAGCAGACTGAGAAGAGGACACGCCCACCTCGGCGGAAACAAGGGC
GTTAGAGGCATCTGCAGCCTGTATCTGGCCGGCCTGTCTAGAGGACTGAGCAGAGTGCATGCCCAGAGATCTCACGCC
GCCACAATGGCCGATGCTGGCATCCCTGATGAGATCCTGTACAGCGACATCAGCGACGACGAGATCATCATCGATGGC
GACGGCGACGGGGATAGCAGCGGAGATGAGGATGACGATGATGGCGGCCTGACAAGACAGGCTGCCAGCAGAATTGCC
ACCGACCTGGGATTTGAGGTGCTGCAGCCTCTGCAGTCTGGCTCTGAGGGCAGAGTGTTCGTGGCTAGAAGGCCTGGC
GAAGCCGATACCGTGGTGCTGAAAGTGGGCCAGAAACCTAGCACACTGATGGAAGGCATGCTGCTGAAGAGACTGGCC
CACGACAACGTGATGAGCCTGAAGCAGATGCTGGCTAGAGGCCCTGTGACCTGTCTGGTGCTGCCTCACTTCAGATGC
GACCTGTACTCCTACCTGACCATGAGAGATGGCCCTCTGGATATGCGCGACGCCGGCAGAGTGATCAGATCTGTGCTG
AGAGGCCTGGCCTACCTGCACGGCATGAGAATCATGCACAGGGACGTGAAGGCCGAGAACATCTTTCTGGAAGATGTG
GACACCGTGTGCCTGGGCGATCTGGGAGCCGCTAGATGTAATGTGGCCGCTCCTAACTTCTACGGCCTGGCCGGAACC
ATCGAGACAAATGCCCCTGAAGTGCTGGCCCGGGACAGATACGATACCAAGGTGGACGTTTGGGGAGCCGGCGTGGTC
CTGTTTGAGACACTGGCTTACCCCAAGACAATCGCTGGCGGCGACGAGCCTGCTATCAATGGCGAGATGCACCTGATC
GACCTGATCAGAGCCCTGGGCGTGCACCCTGAGGAATTCCCTCCAGACACACGGCTGCGGAGCGAGTTCGTTAGATAC
GCCGGAACACACAGACAGCCCTACACACAGTATGCCAGAGTGGCCAGACTGGGCCTGCCTGAAACAGGCGCCTTCCTG
ATCTACAAGATGCTGACCTTCGATCCCGTGCGGAGGCCTTCTGCCGATGAGATTCTGAACTTCGGCATGTGGACCGTG

Hence, preferably the RNA construct is encoded by a DNA sequence substantially as set out in SEQ ID No: 297, or a fragment or variant thereof.

In an embodiment, the RNA sequence corresponding to the codon optimised DNA sequence of SEQ ID No: 297 is provided herein as SEQ ID No: 298, as follows:

[SEQ ID No: 298]
AUGCUGGCCAUGUGGCGCUGGGUCACCAAGAGAAGCAGACUGAGAAGAGGACACGCCCACCUCGGCGGAAACAAGGGC
GUUAGAGGCAUCUGCAGCCUGUAUCUGGCCGGCCUGUCUAGAGGACUGAGCAGAGUGCAUGCCCAGAGAUCUCACGCC
GCCACAAUGGCCGAUGCUGGCAUCCCUGAUGAGAUCCUGUACAGCGACAUCAGCGACGACGAGAUCAUCAUCGAUGGC
GACGGCGACGGGGAUAGCAGCGGAGAUGAGGAUGACGAUGAUGGCGGCCUGACAAGACAGGCUGCCAGCAGAAUUGCC
ACCGACCUGGGAUUUGAGGUGCUGCAGCCUCUGCAGUCUGGCUCUGAGGGCAGAGUGUUCGUGGCUAGAAGGCCUGGC
GAAGCCGAUACCGUGGUGCUGAAAGUGGGCCAGAAACCUAGCACACUGAUGGAAGGCAUGCUGCUGAAGAGACUGGCC
CACGACAACGUGAUGAGCCUGAAGCAGAUGCUGGCUAGAGGCCCUGUGACCUGUCUGGUGCUGCCUCACUUCAGAUGC
GACCUGUACUCCUACCUGACCAUGAGAGAUGGCCCUCUGGAUAUGCGCGACGCCGGCAGAGUGAUCAGAUCUGUGCUG
AGAGGCCUGGCCUACCUGCACGGCAUGAGAAUCAUGCACAGGGACGUGAAGGCCGAGAACAUCUUUCUGGAAGAUGUG
GACACCGUGUGCCUGGGCGAUCUGGGAGCCGCUAGAUGUAAUGUGGCCGCUCCUAACUUCUACGGCCUGGCCGGAACC
AUCGAGACAAAUGCCCCUGAAGUGCUGGCCCGGGACAGAUACGAUACCAAGGUGGACGUUUGGGGAGCCGGCGUGGUC
CUGUUUGAGACACUGGCUUACCCCAAGACAAUCGCUGGCGGCGACGAGCCUGCUAUCAAUGGCGAGAUGCACCUGAUC
GACCUGAUCAGAGCCCUGGGCGUGCACCCUGAGGAAUUCCCUCCAGACACACGGCUGCGGAGCGAGUUCGUUAGAUAC
GCCGGAACACACAGACAGCCCUACACACAGUAUGCCAGAGUGGCCAGACUGGGCCUGCCUGAAACAGGCGCCUUCCUG
AUCUACAAGAUGCUGACCUUCGAUCCCGUGCGGAGGCCUUCUGCCGAUGAGAUUCUGAACUUCGGCAUGUGGACCGUG

Hence, preferably the RNA construct comprises a sequence substantially as set out in SEQ ID No: 298, or a fragment or variant thereof.

In one embodiment, the at least one IIP is HSV1 US3 (P04413; Serine/threonine-protein kinase US3 Human herpesvirus 1 (strain 17)), or an orthologue thereof. One embodiment of the polypeptide sequence of HSV1 US3 is represented herein as SEQ ID No: 299, as follows:

[SEQ ID No: 299]
MACRKFCRVYGGQGRRKEEAVPPETKPSRVFPHGPFYTPAEDACLDSPPPETPKPSHTTPPSEAERLCHLQEILAQMY
GNQDYPIEDDPSADAADDVDEDAPDDVAYPEEYAEELFLPGDATGPLIGANDHIPPPCGASPPGIRRRSRDEIGATGF
TAEELDAMDREAARAISRGGKPPSTMAKLVTGMGFTIHGALTPGSEGCVFDSSHPDYPQRVIVKAGWYTSTSHEARLL
RRLDHPAILPLLDLHVVSGVTCLVLPKYQADLYTYLSRRLNPLGRPQIAAVSROLLSAVDYIHRQGIIHRDIKTENIF
INTPEDICLGDFGAACFVQGSRSSPFPYGIAGTIDTNAPEVLAGDPYTTTVDIWSAGLVIFETAVHNASLFSAPRGPK
RGPCDSQITRIIRQAQVHVDEFSPHPESRLTSRYRSRAAGNNRPPYTRPAWTRYYKMDIDVEYLVCKALTFDGALRPS
AAELLCLPLFQQK

Therefore, preferably the RNA construct comprises a nucleotide sequence which encodes an amino acid sequence substantially as set out in SEQ ID No: 299, or a variant or fragment thereof.

In one embodiment, the HSV1 US3 polypeptide is encoded by the DNA nucleotide sequence of SEQ ID No: 300, as follows:

[SEQ ID No: 300]
ATGGCCTGTCGTAAGTTTTGTCGCGTTTACGGGGGACAGGGCAGGAGGAAGGAGGAGGCCGTCCCGCCGGAGACAAAG
CCGTCCCGGGTGTTTCCTCATGGCCCCTTTTATACCCCAGCCGAGGACGCGTGCCTGGACTCCCCGCCCCCGGAGACC
CCCAAACCTTCCCACACCACACCACCCAGCGAGGCCGAGCGCCTGTGTCATCTGCAGGAGATCCTTGCCCAGATGTAC
GGAAACCAGGACTACCCCATAGAGGACGACCCCAGCGCGGATGCCGCGGACGATGTCGACGAGGACGCCCCGGACGAC
GTGGCCTATCCGGAGGAATACGCAGAGGAGCTTTTTCTGCCCGGGGACGCGACCGGTCCCCTTATCGGGGCCAACGAC
CACATCCCTCCCCCGTGTGGCGCATCTCCCCCCGGTATACGACGACGCAGCCGGGATGAGATTGGGGCCACGGGATTT
ACCGCGGAAGAGCTGGACGCCATGGACAGGGAGGCGGCTCGAGCCATCAGCCGCGGCGGCAAGCCCCCCTCGACCATG
GCCAAGCTGGTGACTGGCATGGGCTTTACGATCCACGGAGCGCTCACCCCAGGATCGGAGGGGTGTGTCTTTGACAGC
AGCCATCCAGATTACCCCCAACGGGTAATCGTGAAGGCGGGGTGGTACACGAGCACGAGCCACGAGGCGCGACTGCTG
AGGCGACTGGACCACCCGGCGATCCTGCCCCTCCTGGACCTGCATGTCGTCTCCGGGGTCACGTGTCTGGTCCTCCCC
AAGTACCAGGCCGACCTGTATACCTATCTGAGTAGGCGCCTGAACCCACTGGGACGCCCGCAGATCGCAGCGGTCTCC
CGGCAGCTCCTAAGCGCCGTTGACTACATTCACCGCCAGGGCATTATCCACCGCGACATTAAGACCGAAAATATTTTT
ATTAACACCCCCGAGGACATTTGCCTGGGGGACTTTGGCGCCGCGTGCTTCGTGCAGGGTTCCCGATCAAGCCCCTTC
CCCTACGGAATCGCCGGAACCATCGACACCAACGCCCCCGAGGTCCTGGCCGGGGATCCGTATACCACGACCGTCGAC
ATTTGGAGCGCCGGTCTGGTGATCTTCGAGACTGCCGTCCACAACGCGTCCTTGTTCTCGGCCCCCCGCGGCCCCAAA
AGGGGCCCGTGCGACAGTCAGATCACCCGCATCATCCGACAGGCCCAGGTCCACGTTGACGAGTTTTCCCCGCATCCA
GAATCGCGCCTCACCTCGCGCTACCGCTCCCGCGCGGCCGGGAACAATCGCCCGCCGTACACCCGACCGGCCTGGACC
CGCTACTACAAGATGGACATAGACGTCGAATATCTGGTTTGCAAAGCCCTCACCTTCGACGGCGCGCTTCGCCCCAGC
GCCGCAGAGCTGCTTTGTTTGCCGCTGTTTCAACAGAAA

Accordingly, preferably the HSV1 US3 polypeptide is encoded by the DNA nucleotide sequence substantially as set out in SEQ ID NO: 300, or a variant or fragment thereof.

One embodiment of codon optimised nucleic acid (DNA) encoding the HSV1 US3 polypeptide is provided herein as SEQ ID No: 301, as follows:

[SEQ ID No: 301]
ATGGCCTGCCGGAAGTTCTGTAGAGTGTACGGCGGACAGGGGCGCAGAAAAGAGGAAGCCGTTCCTCCTGAGACAAAG
CCCAGCAGAGTGTTCCCTCACGGCCCCTTTTACACCCCTGCCGAAGATGCCTGTCTGGACAGCCCTCCTCCAGAAACA
CCTAAGCCTAGCCACACCACACCTCCAAGCGAGGCCGAAAGACTGTGCCATCTGCAAGAGATTCTGGCCCAGATGTAC
GGCAACCAGGACTACCCCATCGAGGACGATCCATCTGCCGATGCCGCCGACGATGTGGATGAAGATGCCCCTGATGAC
GTGGCCTATCCTGAGGAATACGCCGAGGAACTGTTCCTGCCTGGCGACGCTACAGGACCTCTGATCGGAGCCAACGAT
CACATCCCTCCACCTTGTGGCGCTAGCCCTCCTGGCATCAGAAGAAGAAGCAGGGACGAGATCGGCGCCACCGGCTTT
ACAGCCGAAGAACTGGACGCCATGGACAGAGAAGCCGCCAGAGCCATTTCTAGAGGCGGCAAGCCTCCTAGCACCATG
GCCAAACTGGTTACCGGCATGGGCTTCACCATTCACGGCGCTCTGACACCTGGCTCTGAGGGCTGTGTGTTCGACAGC
TCTCACCCCGACTATCCCCAGCGCGTGATCGTGAAAGCCGGCTGGTACACAAGCACAAGCCACGAGGCCAGACTGCTG
CGGAGACTGGATCATCCTGCCATCCTGCCTCTGCTGGATCTGCATGTGGTGTCCGGCGTGACATGTCTGGTGCTGCCT
AAGTACCAGGCCGACCTGTACACCTACCTGAGCAGAAGGCTGAACCCTCTGGGCAGACCTCAGATTGCCGCTGTGTCA
AGACAGCTGCTGAGCGCTGTGGACTACATCCACAGACAGGGCATCATCCACCGGGACATCAAGACCGAGAATATCTTC
ATCAACACGCCCGAGGACATCTGCCTGGGCGATTTTGGCGCCGCTTGCTTCGTGCAAGGCAGCAGAAGCAGCCCCTTT
CCTTATGGAATCGCCGGCACCATCGACACAAACGCCCCTGAAGTTCTGGCCGGCGATCCTTACACCACCACCGTGGAT
ATTTGGAGCGCCGGACTGGTCATCTTCGAGACAGCCGTGCATAACGCCAGCCTGTTCTCTGCCCCTAGAGGCCCTAAA
AGAGGCCCCTGCGATAGCCAGATCACCCGGATCATTAGACAGGCCCAGGTGCACGTGGACGAGTTCTCTCCACATCCT
GAGAGCCGGCTGACCAGCCGGTACAGATCTAGAGCCGCCGGAAACAACCGGCCTCCATACACAAGACCTGCCTGGACA
CGGTACTACAAGATGGACATCGACGTGGAATACCTCGTGTGCAAGGCCCTGACCTTCGATGGCGCCCTTAGACCTTCT
GCCGCCGAACTGCTTTGCCTGCCACTGTTCCAGCAGAAG

Hence, preferably the RNA construct is encoded by a DNA sequence substantially as set out in SEQ ID No: 301, or a fragment or variant thereof.

In an embodiment, the RNA sequence corresponding to the codon optimised DNA sequence of SEQ ID No: 301 is provided herein as SEQ ID No: 302, as follows:

[SEQ ID No: 302]
AUGGCCUGCCGGAAGUUCUGUAGAGUGUACGGCGGACAGGGGCGCAGAAAAGAGGAAGCCGUUCCUCCUGAGACAAAG
CCCAGCAGAGUGUUCCCUCACGGCCCCUUUUACACCCCUGCCGAAGAUGCCUGUCUGGACAGCCCUCCUCCAGAAACA
CCUAAGCCUAGCCACACCACACCUCCAAGCGAGGCCGAAAGACUGUGCCAUCUGCAAGAGAUUCUGGCCCAGAUGUAC
GGCAACCAGGACUACCCCAUCGAGGACGAUCCAUCUGCCGAUGCCGCCGACGAUGUGGAUGAAGAUGCCCCUGAUGAC
GUGGCCUAUCCUGAGGAAUACGCCGAGGAACUGUUCCUGCCUGGCGACGCUACAGGACCUCUGAUCGGAGCCAACGAU
CACAUCCCUCCACCUUGUGGCGCUAGCCCUCCUGGCAUCAGAAGAAGAAGCAGGGACGAGAUCGGCGCCACCGGCUUU
ACAGCCGAAGAACUGGACGCCAUGGACAGAGAAGCCGCCAGAGCCAUUUCUAGAGGCGGCAAGCCUCCUAGCACCAUG
GCCAAACUGGUUACCGGCAUGGGCUUCACCAUUCACGGCGCUCUGACACCUGGCUCUGAGGGCUGUGUGUUCGACAGC
UCUCACCCCGACUAUCCCCAGCGCGUGAUCGUGAAAGCCGGCUGGUACACAAGCACAAGCCACGAGGCCAGACUGCUG
CGGAGACUGGAUCAUCCUGCCAUCCUGCCUCUGCUGGAUCUGCAUGUGGUGUCCGGCGUGACAUGUCUGGUGCUGCCU
AAGUACCAGGCCGACCUGUACACCUACCUGAGCAGAAGGCUGAACCCUCUGGGCAGACCUCAGAUUGCCGCUGUGUCA
AGACAGCUGCUGAGCGCUGUGGACUACAUCCACAGACAGGGCAUCAUCCACCGGGACAUCAAGACCGAGAAUAUCUUC
AUCAACACGCCCGAGGACAUCUGCCUGGGCGAUUUUGGCGCCGCUUGCUUCGUGCAAGGCAGCAGAAGCAGCCCCUUU
CCUUAUGGAAUCGCCGGCACCAUCGACACAAACGCCCCUGAAGUUCUGGCCGGCGAUCCUUACACCACCACCGUGGAU
AUUUGGAGCGCCGGACUGGUCAUCUUCGAGACAGCCGUGCAUAACGCCAGCCUGUUCUCUGCCCCUAGAGGCCCUAAA
AGAGGCCCCUGCGAUAGCCAGAUCACCCGGAUCAUUAGACAGGCCCAGGUGCACGUGGACGAGUUCUCUCCACAUCCU
GAGAGCCGGCUGACCAGCCGGUACAGAUCUAGAGCCGCCGGAAACAACCGGCCUCCAUACACAAGACCUGCCUGGACA
CGGUACUACAAGAUGGACAUCGACGUGGAAUACCUCGUGUGCAAGGCCCUGACCUUCGAUGGCGCCCUUAGACCUUCU
GCCGCCGAACUGCUUUGCCUGCCACUGUUCCAGCAGAAG

Hence, preferably the RNA construct comprises a sequence substantially as set out in SEQ ID No: 302, or a fragment or variant thereof.

In one embodiment, the at least one HP is HSV2 US1 (A0A290Y3Z3; A0A290Y3Z3 HHV2 US1 Human herpesvirus 2), or an orthologue thereof. One embodiment of the polypeptide sequence of HSV2 US1 is represented herein as SEQ ID No: 303, as follows:

[SEQ ID No: 303]
MADIPPDPPALNTTPVNHAPPSPPPGSRKRRRPVLPSSSESEGKPDTESESSSTESSEDEAGDLRGGRRRSPRELGGR
YFLDLSAESTTGTESEGTGPSDDDDDDASDGWLVDTPPRKSKRPRINLRLTSSPDRRAGVVFPEVWRNDRPIRAAQPQ
APAQSSGDRAAAPRRSARQAQMRSGAAWTLDLHYIRQCVNQLFRILRAAPNPPGSANRLRHLVRDCYLMGYCRTRLGP
RTWGRLLQISGGTWDVRLRNAIREVEARFEPAAEPVCELPCLNARRYGPECDVGNLETNGGSTSDDEISDATDSDDTL
ASHSDTEGGPSPAGRENPESASGGAIAARLECEFGTFDWTSEEGSQPWLSAVVADTSSAERSGLPAPGACRATEAPER
EDGCRKMRFPAACPYPCGHTFLRP

Therefore, preferably the RNA construct comprises a nucleotide sequence which encodes an amino acid sequence substantially as set out in SEQ ID No: 303, or a variant or fragment thereof.

In one embodiment, the HSV2 US1 polypeptide is encoded by the DNA nucleotide sequence of SEQ ID No: 304, as follows:

[SEQ ID No: 304]
ATGGCAGACATCCCCCCGGACCCGCCCGCGCTCAACACGACGCCTGTGAATCATGCTCCCCCATCCCCGCCCCCGGGT
TCACGGAAGCGCAGACGCCCCGTCCTCCCCAGCTCGTCGGAATCTGAGGGTAAGCCCGACACAGAATCGGAATCCTCC
TCGACCGAGTCGTCCGAGGATGAGGCGGGAGACCTACGCGGCGGGCGCCGTCGCTCCCCGCGGGAGCTCGGGGGGAGG
TATTTTTTGGATCTGTCGGCAGAATCGACCACGGGGACGGAATCGGAGGGAACGGGGCCGTCGGACGACGATGATGAT
GATGCGTCAGACGGCTGGTTGGTTGACACCCCCCCCCGTAAATCCAAGCGACCCCGAATCAACCTGCGATTAACGAGC
TCCCCCGACCGGCGCGCGGGTGTGGTTTTCCCCGAGGTGTGGAGAAACGACAGACCTATCCGCGCGGCGCAACCCCAG
GCCCCGGCCCAGTCTTCCGGGGATCGCGCAGCCGCACCGCGGCGCTCTGCTCGCCAGGCCCAGATGCGGAGCGGAGCC
GCCTGGACGCTTGATCTGCATTACATACGCCAGTGCGTCAACCAGCTCTTTCGGATCCTGCGTGCCGCCCCGAACCCG
CCCGGCAGCGCCAACCGCCTGCGCCACCTGGTGCGAGACTGCTACCTCATGGGCTACTGCCGGACCCGCCTGGGGCCG
CGCACGTGGGGCCGCCTGCTGCAGATCTCGGGCGGAACCTGGGACGTGCGCCTGCGAAACGCAATCCGGGAGGTCGAG
GCGCGTTTTGAACCCGCCGCCGAGCCCGTGTGCGAGCTGCCCTGTCTGAACGCCAGGCGTTACGGCCCCGAGTGTGAT
GTTGGCAATCTCGAGACCAACGGCGGCTCGACGAGCGATGATGAGATATCGGATGCGACGGACTCGGACGATACCCTC
GCGTCCCATTCCGACACGGAGGGGGGGCCCTCCCCGGCCGGCCGGGAGAACCCGGAATCCGCGTCCGGCGGGGCTATC
GCGGCTCGGCTGGAGTGTGAGTTTGGGACGTTTGACTGGACGTCCGAGGAGGGCTCCCAGCCCTGGCTGTCCGCGGTG
GTCGCCGATACCAGCTCCGCCGAACGCTCTGGCCTACCCGCCCCGGGCGCGTGTCGCGCAACGGAAGCCCCAGAACGC
GAGGACGGGTGCCGAAAAATGCGCTTCCCCGCCGCCTGCCCCTATCCCTGCGGCCACACATTTCTCCGGCCA

Accordingly, preferably the HSV2 US1 polypeptide is encoded by the DNA nucleotide sequence substantially as set out in SEQ ID NO: 304, or a variant or fragment thereof.

One embodiment of codon optimised nucleic acid (DNA) encoding the HSV2 US1 polypeptide is provided herein as SEQ ID No: 305, as follows:

[SEQ ID No: 305]
ATGGCCGACATTCCTCCTGATCCTCCAGCTCTGAACACCACACCTGTGAATCACGCCCCTCCATCTCCACCACCTGGC
AGCAGAAAGAGAAGAAGGCCTGTCCTGCCTAGCAGCAGCGAGTCTGAGGGCAAGCCTGATACAGAGAGCGAGAGCAGC
AGCACAGAGAGCAGCGAGGACGAAGCTGGCGATCTTAGAGGCGGCAGAAGAAGAAGCCCCAGAGAACTCGGCGGCAGA
TACTTCCTGGATCTGAGCGCCGAGAGCACCACCGGCACTGAATCTGAAGGCACAGGCCCCAGCGACGACGATGACGAT
GATGCCTCTGATGGCTGGCTGGTGGACACCCCTCCTAGAAAGTCCAAGCGGCCCAGAATCAACCTGCGGCTGACAAGC
TCTCCTGATCGCAGAGCTGGCGTGGTGTTCCCCGAAGTGTGGCGGAACGACAGACCTATCAGAGCCGCTCAGCCTCAG
GCTCCTGCTCAGTCTAGCGGAGATAGAGCTGCCGCTCCTAGAAGATCTGCCAGACAGGCCCAGATGAGAAGCGGAGCT
GCTTGGACACTGGACCTGCACTACATCCGGCAGTGCGTGAACCAGCTGTTCCGGATCCTTCGGGCTGCCCCTAATCCA
CCTGGCTCCGCCAATAGACTGAGACACCTTGTGCGGGACTGCTACCTGATGGGCTACTGCAGAACAAGACTGGGCCCC
AGAACATGGGGCAGACTGCTGCAAATCTCTGGCGGCACATGGGACGTGCGGCTGAGAAACGCCATTAGAGAGGTGGAA
GCCAGATTCGAGCCAGCCGCTGAGCCTGTGTGTGAACTGCCTTGTCTGAACGCTCGGAGATACGGCCCCGAGTGTGAT
GTGGGCAACCTGGAAACAAATGGCGGCAGCACCTCCGACGACGAGATCTCTGATGCCACCGACAGCGACGATACACTG
GCCAGCCACAGCGATACAGAAGGCGGACCATCTCCTGCCGGAAGAGAGAATCCTGAGTCTGCCTCTGGCGGAGCTATC
GCCGCAAGACTGGAATGCGAGTTCGGCACCTTCGACTGGACAAGCGAGGAAGGCTCTCAGCCTTGGCTGTCTGCTGTG
GTGGCCGATACCTCTAGCGCCGAAAGATCTGGACTTCCTGCTCCTGGCGCCTGCAGAGCTACAGAAGCTCCTGAAAGA
GAGGACGGCTGCAGAAAGATGCGGTTCCCTGCCGCCTGTCCTTATCCTTGCGGCCACACATTTCTGCGGCCT

Hence, preferably the RNA construct is encoded by a DNA sequence substantially as set out in SEQ ID No: 305, or a fragment or variant thereof.

In an embodiment, the RNA sequence corresponding to the codon optimised DNA sequence of SEQ ID No: 305 is provided herein as SEQ ID No: 306, as follows:

[SEQ ID No: 306]
AUGGCCGACAUUCCUCCUGAUCCUCCAGCUCUGAACACCACACCUGUGAAUCACGCCCCUCCAUCUCCACCACCUGGC
AGCAGAAAGAGAAGAAGGCCUGUCCUGCCUAGCAGCAGCGAGUCUGAGGGCAAGCCUGAUACAGAGAGCGAGAGCAGC
AGCACAGAGAGCAGCGAGGACGAAGCUGGCGAUCUUAGAGGCGGCAGAAGAAGAAGCCCCAGAGAACUCGGCGGCAGA
UACUUCCUGGAUCUGAGCGCCGAGAGCACCACCGGCACUGAAUCUGAAGGCACAGGCCCCAGCGACGACGAUGACGAU
GAUGCCUCUGAUGGCUGGCUGGUGGACACCCCUCCUAGAAAGUCCAAGCGGCCCAGAAUCAACCUGCGGCUGACAAGC
UCUCCUGAUCGCAGAGCUGGCGUGGUGUUCCCCGAAGUGUGGCGGAACGACAGACCUAUCAGAGCCGCUCAGCCUCAG
GCUCCUGCUCAGUCUAGCGGAGAUAGAGCUGCCGCUCCUAGAAGAUCUGCCAGACAGGCCCAGAUGAGAAGCGGAGCU
GCUUGGACACUGGACCUGCACUACAUCCGGCAGUGCGUGAACCAGCUGUUCCGGAUCCUUCGGGCUGCCCCUAAUCCA
CCUGGCUCCGCCAAUAGACUGAGACACCUUGUGCGGGACUGCUACCUGAUGGGCUACUGCAGAACAAGACUGGGCCCC
AGAACAUGGGGCAGACUGCUGCAAAUCUCUGGCGGCACAUGGGACGUGCGGCUGAGAAACGCCAUUAGAGAGGUGGAA
GCCAGAUUCGAGCCAGCCGCUGAGCCUGUGUGUGAACUGCCUUGUCUGAACGCUCGGAGAUACGGCCCCGAGUGUGAU
GUGGGCAACCUGGAAACAAAUGGCGGCAGCACCUCCGACGACGAGAUCUCUGAUGCCACCGACAGCGACGAUACACUG
GCCAGCCACAGCGAUACAGAAGGCGGACCAUCUCCUGCCGGAAGAGAGAAUCCUGAGUCUGCCUCUGGCGGAGCUAUC
GCCGCAAGACUGGAAUGCGAGUUCGGCACCUUCGACUGGACAAGCGAGGAAGGCUCUCAGCCUUGGCUGUCUGCUGUG
GUGGCCGAUACCUCUAGCGCCGAAAGAUCUGGACUUCCUGCUCCUGGCGCCUGCAGAGCUACAGAAGCUCCUGAAAGA
GAGGACGGCUGCAGAAAGAUGCGGUUCCCUGCCGCCUGUCCUUAUCCUUGCGGCCACACAUUUCUGCGGCCU

Hence, preferably the RNA construct comprises a sequence substantially as set out in SEQ ID No: 306, or a fragment or variant thereof.

In one embodiment, the at least one IIP is CSFV NPro (P19712; Genome polyprotein Classical swine fever virus (strain Alfort)), or an orthologue thereof. One embodiment of the polypeptide sequence of CSFV NPro is represented herein as SEQ ID No: 307, as follows:

[SEQ ID No: 307]
MELNHFELLYKTSKQKPVGVEEPVYDTAGRPLFGNPSEVHPQSTLKLPHDRGRGDIRTTLRDLPRKGDCRSGNHLGPV
SGIYIKPGPVYYQDYTGPVYHRAPLEFFDEAQFCEVTKRIGRVTGSDGKLYHIYVCVDGCILLKLAKRGTPRTLKWIR
NFTNCPLWVTSC

Therefore, preferably the RNA construct comprises a nucleotide sequence which encodes an amino acid sequence substantially as set out in SEQ ID No: 307, or a variant or fragment thereof.

In one embodiment, the CSFV NPro polypeptide is encoded by the DNA nucleotide sequence of SEQ ID No: 308, as follows:

[SEQ ID No: 308]
ATGGAGTTGAATCATTTTGAATTATTATACAAAACAAGCAAACAAAAACCAGTGGGAGTGGAGGAACCGGTGTATGAC
ACCGCGGGGAGACCACTATTTGGGAACCCAAGTGAGGTACACCCACAATCAACGCTGAAGCTGCCACACGACAGGGGG
AGAGGAGATATCAGAACAACACTGAGGGACCTACCCAGGAAAGGTGACTGTAGGAGTGGCAACCATCTAGGCCCGGTT
AGTGGGATATACATAAAGCCCGGCCCTGTCTACTATCAGGACTACACGGGCCCAGTCTATCACAGAGCTCCTTTAGAG
TTCTTTGATGAGGCCCAGTTCTGCGAGGTGACTAAGAGAATAGGCAGGGTCACGGGTAGTGATGGTAAGCTTTACCAC
ATATATGTGTGCGTCGATGGTTGCATACTGCTGAAATTAGCCAAAAGGGGCACACCCAGAACCCTAAAGTGGATTAGG
AACTTCACCAACTGTCCATTATGGGTAACCAGTTGC

Accordingly, preferably the CSFV NPro polypeptide is encoded by the DNA nucleotide sequence substantially as set out in SEQ ID NO: 308, or a variant or fragment thereof.

One embodiment of codon optimised nucleic acid (DNA) encoding the CSFV NPro polypeptide is provided herein as SEQ ID No: 309, as follows:

[SEQ ID No: 309]
ATGGAACTGAACCACTTCGAGCTGCTGTACAAGACCAGCAAGCAGAAACCCGTGGGCGTCGAGGAACCCGTGTATGAT
ACAGCTGGCAGACCCCTGTTCGGCAACCCCTCTGAAGTGCACCCTCAGAGCACACTGAAGCTGCCCCACGATAGAGGC
AGAGGCGACATCAGAACCACACTGCGGGACCTGCCTAGAAAGGGCGATTGCAGAAGCGGCAATCATCTGGGCCCTGTG
TCCGGCATCTACATCAAGCCTGGACCAGTGTACTACCAGGACTACACAGGCCCCGTGTACCACAGAGCCCCTCTGGAA
TTCTTCGACGAGGCCCAGTTCTGCGAAGTGACCAAGAGAATCGGCAGAGTGACCGGCTCCGACGGCAAGCTGTACCAC
ATCTACGTGTGCGTGGACGGCTGCATCCTGCTGAAGCTGGCCAAGAGAGGCACCCCTAGAACACTGAAGTGGATCCGG
AACTTCACCAACTGTCCTCTGTGGGTCACCAGCTGC

Hence, preferably the RNA construct is encoded by a DNA sequence substantially as set out in SEQ ID No: 309, or a fragment or variant thereof.

In an embodiment, the RNA sequence corresponding to the codon optimised DNA sequence of SEQ ID No: 309 is provided herein as SEQ ID No: 310, as follows:

[SEQ ID No: 310]
AUGGAACUGAACCACUUCGAGCUGCUGUACAAGACCAGCAAGCAGAAACCCGUGGGCGUCGAGGAACCCGUGUAUGAU
ACAGCUGGCAGACCCCUGUUCGGCAACCCCUCUGAAGUGCACCCUCAGAGCACACUGAAGCUGCCCCACGAUAGAGGC
AGAGGCGACAUCAGAACCACACUGCGGGACCUGCCUAGAAAGGGCGAUUGCAGAAGCGGCAAUCAUCUGGGCCCUGUG
UCCGGCAUCUACAUCAAGCCUGGACCAGUGUACUACCAGGACUACACAGGCCCCGUGUACCACAGAGCCCCUCUGGAA
UUCUUCGACGAGGCCCAGUUCUGCGAAGUGACCAAGAGAAUCGGCAGAGUGACCGGCUCCGACGGCAAGCUGUACCAC
AUCUACGUGUGCGUGGACGGCUGCAUCCUGCUGAAGCUGGCCAAGAGAGGCACCCCUAGAACACUGAAGUGGAUCCGG
AACUUCACCAACUGUCCUCUGUGGGUCACCAGCUGC

Hence, preferably the RNA construct comprises a sequence substantially as set out in SEQ ID No: 310, or a fragment or variant thereof.

In one embodiment, the at least one IIP is BDV Npro (A0A290YXI0; Autoprotease p20 Border disease virus), or an orthologue thereof. One embodiment of the polypeptide sequence of BDV Npro is represented herein as SEQ ID No: 311, as follows:

[SEQ ID No: 311]
MELNKFELLYKTSKQRPVGAVEPVYDSAGNPLYGERTTVHPQATLKLPHHRGVAEVITTLKDLPRKGDCRSGNHRGPV
SGIYIKPGPVIYQDYKRPVYHRAPLEQFTKVQICEATKRVGRVTGSDGKLYHLYVCMDGCILLKLASRTVNAVLKWTH
NTLDCPLWVTSC

Therefore, preferably the RNA construct comprises a nucleotide sequence which encodes an amino acid sequence substantially as set out in SEQ ID No: 311, or a variant or fragment thereof.

In one embodiment, the BDV Npro polypeptide is encoded by the DNA nucleotide sequence of SEQ ID No: 312, as follows:

[SEQ ID No: 312]
ATGGAGTTGAATAAGTTTGAACTTTTATACAAAACAAGTAAACAAAGACCAGTAGGGGCTGTTGAACCAGTTTATGAC
TCAGCGGGTAACCCCCTATATGGTGAAAGAACAACAGTACACCCGCAAGCCACTCTGAAACTACCACATCACAGGGGA
GTAGCCGAGGTGATAACAACCCTGAAGGATTTGCCCAGGAAAGGAGACTGCAGGAGTGGAAACCATCGAGGCCCAGTG
AGTGGTATATACATCAAGCCAGGTCCAGTCATATACCAGGATTACAAGAGACCGGTGTACCACAGGGCTCCTCTGGAG
CAGTTCACGAAGGTACAAATCTGTGAGGCTACGAAAAGGGTGGGGAGAGTCACTGGCAGCGATGGCAAATTGTACCAC
CTATACGTTTGCATGGATGGTTGCATATTGCTGAAACTGGCAAGCAGGACCGTGAATGCAGTGCTAAAATGGACACAT
AACACTCTGGACTGTCCACTTTGGGTTACAAGCTGC

Accordingly, preferably the BDV Npro polypeptide is encoded by the DNA nucleotide sequence substantially as set out in SEQ ID NO: 312, or a variant or fragment thereof.

One embodiment of codon optimised nucleic acid (DNA) encoding the BDV Npro polypeptide is provided herein as SEQ ID No: 313, as follows:

[SEQ ID No: 313]
ATGGAACTGAACAAGTTCGAGCTGCTGTACAAGACCAGCAAGCAGAGGCCTGTGGGAGCCGTGGAACCTGTGTATGAT
AGCGCCGGCAATCCCCTGTACGGCGAGAGAACAACAGTGCACCCTCAGGCCACACTGAAGCTGCCTCATCATAGAGGC
GTGGCCGAAGTGATCACAACCCTGAAGGACCTGCCTCGGAAGGGCGATTGCAGAAGCGGCAATCACAGAGGCCCTGTG
TCCGGCATCTACATCAAGCCCGGACCTGTGATCTACCAGGACTACAAGCGGCCCGTGTACCACAGAGCACCCCTGGAA
CAGTTCACCAAGGTGCAGATTTGCGAGGCCACCAAGCGCGTGGGAAGAGTGACAGGCTCTGACGGCAAGCTGTACCAC
CTGTACGTGTGCATGGACGGCTGCATCCTGCTGAAACTGGCCAGCAGAACCGTGAACGCCGTGCTGAAGTGGACCCAC
AACACCCTGGATTGCCCTCTGTGGGTCACCAGCTGT

Hence, preferably the RNA construct is encoded by a DNA sequence substantially as set out in SEQ ID No: 313, or a fragment or variant thereof.

In an embodiment, the RNA sequence corresponding to the codon optimised DNA sequence of SEQ ID No: 313 is provided herein as SEQ ID No: 314, as follows:

[SEQ ID No: 314]
AUGGAACUGAACAAGUUCGAGCUGCUGUACAAGACCAGCAAGCAGAGGCCUGUGGGAGCCGUGGAACCUGUGUAUGAU
AGCGCCGGCAAUCCCCUGUACGGCGAGAGAACAACAGUGCACCCUCAGGCCACACUGAAGCUGCCUCAUCAUAGAGGC
GUGGCCGAAGUGAUCACAACCCUGAAGGACCUGCCUCGGAAGGGCGAUUGCAGAAGCGGCAAUCACAGAGGCCCUGUG
UCCGGCAUCUACAUCAAGCCCGGACCUGUGAUCUACCAGGACUACAAGCGGCCCGUGUACCACAGAGCACCCCUGGAA
CAGUUCACCAAGGUGCAGAUUUGCGAGGCCACCAAGCGCGUGGGAAGAGUGACAGGCUCUGACGGCAAGCUGUACCAC
CUGUACGUGUGCAUGGACGGCUGCAUCCUGCUGAAACUGGCCAGCAGAACCGUGAACGCCGUGCUGAAGUGGACCCAC
AACACCCUGGAUUGCCCUCUGUGGGUCACCAGCUGU

Hence, preferably the RNA construct comprises a sequence substantially as set out in SEQ ID No: 314, or a fragment or variant thereof.

In one embodiment, the at least one IIP is Bovine RV NS1 (Q8JZ13; Non-structural protein 1 Bovine Rotavirus A), or an orthologue thereof. One embodiment of the polypeptide sequence of Bovine RV NS1 is represented herein as SEQ ID No: 315, as follows:

[SEQ ID No: 315]
MATFKDACYHYKKLNKLNSLVLKLGANDEWRPAPVTKYKGWCLDCCQYTN
LTYCRGCALYHVCQWCSQYNRCFLDEEPHLLRMRTFKDVVTKEDIEGLLT
MYETLFPINEKLVNKFINSVKQRKCRNEYLLEWYNHLLMPITLQALTINL
EDNVYYMFGYYDCMEHENQTPFQFVNLLEKYDKLLLDDRNFHRMSHLPVI
LQQEYALRYFSKSRFLSKGKKRLSRSDFSDNLMEDRHSPTSLMQVVRNCI
SIHIDDCEWNKACTLIVDARNYISIMNSSYTEHYSVSQRCKLFTKYKFGI
VSKLVKPNYIFSSHESCALNVHNCKWCQINNHYKVWEDFRLRKIYNNVMD
FIRALVKSNVNVGHCSSQESVYKYVPDLFLICKTEKWSEAVEMLFNYLEP
VNVNGTEYVLLDYEVNWEVRGLVMQNMDGKVPRILNMNDTKKILSAMIFD
WFDTRYMRETPMTTSTTNQLRTLNKRNELIDEYDLELSDVE

Therefore, preferably the RNA construct comprises a nucleotide sequence which encodes an amino acid sequence substantially as set out in SEQ ID No: 315, or a variant or fragment thereof.

In one embodiment, the Bovine RV NS1 polypeptide is encoded by the DNA nucleotide sequence of SEQ ID No: 316, as follows:

[SEQ ID No: 316]
ATGGCGACTTTTAAGGACGCTTGTTATCATTATAAAAAGTTGAATAAATT
AAATAGTTTAGTGCTCAAACTAGGAGCAAATGATGAATGGAGGCCAGCAC
CAGTGACAAAATATAAAGGATGGTGTTTAGATTGTTGTCAATATACAAAT
TTGACATATTGCAGAGGGTGCGCTCTATACCATGTATGTCAGTGGTGCAG
TCAGTATAACAGGTGTTTCTTAGATGAAGAACCCCATTTGCTGAGAATGC
GAACATTTAAAGATGTAGTAACAAAAGAAGATATAGAAGGACTGCTAACC
ATGTATGAAACGTTGTTTCCAATAAATGAAAAGTTAGTGAATAAATTCAT
AAACTCTGTGAAGCAGCGTAAGTGTAGGAATGAGTATTTGTTAGAATGGT
ATAACCACTTACTAATGCCAATAACATTGCAAGCATTGACTATAAATCTT
GAGGATAATGTATATTATATGTTTGGATACTATGATTGCATGGAGCATGA
AAACCAAACACCATTCCAATTTGTTAACCTACTAGAAAAATATGATAAAT
TGCTACTAGATGATAGAAATTTCCATAGAATGTCACACTTACCAGTAATA
TTGCAACAAGAGTATGCGTTGAGATATTTTTCAAAATCAAGATTTTTAAG
TAAAGGGAAGAAAAGATTGAGTAGGAGTGATTTCTCAGATAATCTTATGG
AAGATAGACATAGTCCAACATCATTAATGCAAGTGGTACGTAACTGCATC
AGTATACACATAGATGATTGTGAATGGAATAAAGCGTGTACGCTTATAGT
TGATGCTAGAAATTATATTAGTATTATGAATTCATCGTATACTGAGCATT
ACAGTGTGTCACAAAGATGTAAACTGTTCACTAAGTATAAATTTGGGATT
GTATCAAAATTGGTGAAACCGAATTACATTTTTTCTAGCCATGAATCATG
CGCATTAAACGTACACAATTGTAAATGGTGTCAGATCAATAACCATTACA
AAGTGTGGGAAGATTTTAGACTTAGGAAAATATACAATAATGTAATGGAT
TTTATCAGGGCACTTGTGAAATCGAATGTAAACGTTGGACATTGTTCATC
ACAGGAATCAGTGTATAAGTATGTACCGGATTTATTTTTAATTTGTAAAA
CGGAAAAATGGAGCGAAGCTGTCGAAATGTTATTTAATTATCTAGAACCA
GTGAACGTAAATGGAACGGAGTATGTATTATTAGACTATGAAGTGAACTG
GGAAGTGAGGGGACTAGTCATGCAAAACATGGACGGGAAAGTACCAAGAA
TTTTGAATATGAATGATACAAAGAAGATACTGAGTGCAATGATATTTGAC
TGGTTTGACACAAGATATATGAGAGAAACACCAATGACGACGTCAACAAC
AAACCAACTTCGAACACTGAACAAAAGGAATGAGCTCATAGATGAGTACG
ATTTAGAACTTTCAGATGTTGAA

Accordingly, preferably the Bovine RV NS1 polypeptide is encoded by the DNA nucleotide sequence substantially as set out in SEQ ID NO: 316, or a variant or fragment thereof.

One embodiment of codon optimised nucleic acid (DNA) encoding the Bovine RV NS1 polypeptide is provided herein as SEQ ID No: 317, as follows:

[SEQ ID No: 317]
ATGGCCACCTTCAAGGACGCCTGCTACCACTACAAGAAGCTGAACAAGCT
GAATAGCCTGGTGCTGAAGCTGGGCGCCAATGATGAATGGCGACCTGCTC
CTGTGACCAAGTACAAAGGCTGGTGCCTGGACTGCTGCCAGTACACCAAT
CTGACCTACTGCAGAGGCTGCGCCCTGTACCACGTCTGTCAGTGGTGCAG
CCAGTACAACCGGTGCTTCCTGGACGAGGAACCCCATCTGCTGCGGATGC
GGACCTTTAAGGACGTGGTCACCAAAGAGGACATCGAGGGCCTGCTGACT
ATGTACGAGACACTGTTCCCCATCAACGAGAAGCTGGTCAACAAGTTCAT
CAACAGCGTGAAGCAGCGGAAGTGCCGGAACGAGTACCTGCTGGAATGGT
ACAATCATCTGCTGATGCCCATCACACTGCAGGCCCTGACCATCAACCTG
GAAGATAACGTGTACTACATGTTCGGCTACTACGACTGCATGGAACACGA
GAATCAGACCCCTTTCCAGTTCGTGAACCTGCTCGAGAAGTACGACAAGC
TGCTGCTGGACGACCGGAACTTCCACCGGATGTCTCATCTGCCCGTGATC
CTGCAGCAAGAGTACGCCCTGCGGTACTTCAGCAAGAGCCGGTTTCTGAG
CAAGGGCAAGAAGCGGCTGAGCAGAAGCGACTTCAGCGACAACCTGATGG
AAGATCGGCACAGCCCCACCAGCCTGATGCAGGTCGTCAGAAACTGCATC
AGCATCCACATCGACGACTGTGAATGGAACAAGGCCTGCACACTGATCGT
GGACGCCCGCAACTACATCTCCATCATGAACAGCAGCTACACCGAGCACT
ACAGCGTGTCCCAGCGGTGCAAGCTGTTCACAAAGTACAAGTTCGGCATC
GTGTCCAAGCTCGTGAAGCCCAATTACATCTTCAGCAGCCACGAGAGCTG
TGCCCTGAACGTGCACAACTGCAAGTGGTGCCAGATCAACAATCACTACA
AAGTGTGGGAAGATTTCCGGCTGCGGAAGATCTACAACAACGTGATGGAC
TTCATCCGCGCTCTGGTCAAGAGCAACGTGAACGTGGGCCACTGCAGCAG
CCAAGAGTCCGTGTACAAATACGTGCCCGACCTGTTCCTGATCTGCAAGA
CCGAGAAGTGGAGCGAGGCCGTGGAAATGCTGTTCAACTACCTGGAACCT
GTGAACGTCAACGGCACCGAGTACGTCCTGCTGGACTACGAAGTGAACTG
GGAAGTGCGGGGCCTCGTGATGCAGAACATGGATGGCAAGGTGCCCCGGA
TCCTGAACATGAACGACACCAAGAAAATCCTGAGCGCCATGATCTTCGAT
TGGTTCGACACCCGGTACATGCGCGAGACACCTATGACCACCAGCACCAC
CAACCAGCTGCGGACCCTGAACAAGAGAAACGAGCTGATCGACGAGTACG
ACCTGGAACTGAGCGACGTGGAA

Hence, preferably the RNA construct is encoded by a DNA sequence substantially as set out in SEQ ID No: 317, or a fragment or variant thereof.

In an embodiment, the RNA sequence corresponding to the codon optimised DNA sequence of SEQ ID No: 317 is provided herein as SEQ ID No: 318, as follows:

[SEQ ID No: 318]
AUGGCCACCUUCAAGGACGCCUGCUACCACUACAAGAAGCUGAACAAGCU
GAAUAGCCUGGUGCUGAAGCUGGGCGCCAAUGAUGAAUGGCGACCUGCUC
CUGUGACCAAGUACAAAGGCUGGUGCCUGGACUGCUGCCAGUACACCAAU
CUGACCUACUGCAGAGGCUGCGCCCUGUACCACGUCUGUCAGUGGUGCAG
CCAGUACAACCGGUGCUUCCUGGACGAGGAACCCCAUCUGCUGCGGAUGC
GGACCUUUAAGGACGUGGUCACCAAAGAGGACAUCGAGGGCCUGCUGACU
AUGUACGAGACACUGUUCCCCAUCAACGAGAAGCUGGUCAACAAGUUCAU
CAACAGCGUGAAGCAGCGGAAGUGCCGGAACGAGUACCUGCUGGAAUGGU
ACAAUCAUCUGCUGAUGCCCAUCACACUGCAGGCCCUGACCAUCAACCUG
GAAGAUAACGUGUACUACAUGUUCGGCUACUACGACUGCAUGGAACACGA
GAAUCAGACCCCUUUCCAGUUCGUGAACCUGCUCGAGAAGUACGACAAGC
UGCUGCUGGACGACCGGAACUUCCACCGGAUGUCUCAUCUGCCCGUGAUC
CUGCAGCAAGAGUACGCCCUGCGGUACUUCAGCAAGAGCCGGUUUCUGAG
CAAGGGCAAGAAGCGGCUGAGCAGAAGCGACUUCAGCGACAACCUGAUGG
AAGAUCGGCACAGCCCCACCAGCCUGAUGCAGGUCGUCAGAAACUGCAUC
AGCAUCCACAUCGACGACUGUGAAUGGAACAAGGCCUGCACACUGAUCGU
GGACGCCCGCAACUACAUCUCCAUCAUGAACAGCAGCUACACCGAGCACU
ACAGCGUGUCCCAGCGGUGCAAGCUGUUCACAAAGUACAAGUUCGGCAUC
GUGUCCAAGCUCGUGAAGCCCAAUUACAUCUUCAGCAGCCACGAGAGCUG
UGCCCUGAACGUGCACAACUGCAAGUGGUGCCAGAUCAACAAUCACUACA
AAGUGUGGGAAGAUUUCCGGCUGCGGAAGAUCUACAACAACGUGAUGGAC
UUCAUCCGCGCUCUGGUCAAGAGCAACGUGAACGUGGGCCACUGCAGCAG
CCAAGAGUCCGUGUACAAAUACGUGCCCGACCUGUUCCUGAUCUGCAAGA
CCGAGAAGUGGAGCGAGGCCGUGGAAAUGCUGUUCAACUACCUGGAACCU
GUGAACGUCAACGGCACCGAGUACGUCCUGCUGGACUACGAAGUGAACUG
GGAAGUGCGGGGCCUCGUGAUGCAGAACAUGGAUGGCAAGGUGCCCCGGA
UCCUGAACAUGAACGACACCAAGAAAAUCCUGAGCGCCAUGAUCUUCGAU
UGGUUCGACACCCGGUACAUGCGCGAGACACCUAUGACCACCAGCACCAC
CAACCAGCUGCGGACCCUGAACAAGAGAAACGAGCUGAUCGACGAGUACG
ACCUGGAACUGAGCGACGUGGAA

Hence, preferably the RNA construct comprises a sequence substantially as set out in SEQ ID No: 318, or a fragment or variant thereof.

In one embodiment, the at least one IIP is Bovine RV NS2 (Q86505; Non-structural protein 2 Bovine Rotavirus A), or an orthologue thereof. One embodiment of the polypeptide sequence of Bovine RV NS2 is represented herein as SEQ ID No: 319, as follows:

[SEQ ID No: 319]
MAELACFCYPHLESDTYRFIPFNSLAIKCMLTAKVDKKDQDKFYNSIIYG
IAPPPQFKKRYNTNDNSRGMNYETPMFNKVAVLICEALNSIKVTQSDVAS
VLSKVISVRHLENLVLRRENHQDVLFHSKELLLRSVLIAIGHSKEIETTA
TAEGGEVVFQNAAFTMWKLTYLEHRLMPILDQNFIEYKITVNEDKPISES
HVRELIAELRWQYNKFAVITHGKGHYRVVKYSSVANHADRVYATFKSNNK
NGNVIEFNLLDQRIIWQNWYAFTSSMKQGNTLEICKKLLFQKMKRESNPF
KGLSTDRKMDEVSQIGI

Therefore, preferably the RNA construct comprises a nucleotide sequence which encodes an amino acid sequence substantially as set out in SEQ ID No: 319, or a variant or fragment thereof.

In one embodiment, the Bovine RV NS2 polypeptide is encoded by the DNA nucleotide sequence of SEQ ID No: 320, as follows:

[SEQ ID No: 320]
ATGGCTGAGCTAGCTTGCTTTTGTTATCCCCATTTGGAGAGCGATACGTA
TAGATTCATTCCATTTAACAGTTTAGCTATAAAATGTATGTTGACAGCAA
AAGTGGACAAAAAAGATCAGGATAAGTTTTACAATTCAATAATTTATGGC
ATTGCACCACCGCCACAGTTCAAAAAACGTTATAACACAAATGATAATTC
GAGAGGAATGAATTATGAAACTCCAATGTTTAATAAAGTGGCGGTATTAA
TTTGTGAAGCGTTGAATTCAATTAAAGTTACTCAATCTGATGTTGCGAGT
GTACTTTCAAAAGTAATTTCTGTAAGACATTTAGAGAATTTGGTACTGAG
AAGAGAGAACCATCAGGACGTGCTTTTCCATTCAAAAGAGTTGTTGCTGA
GATCAGTACTAATAGCTATTGGTCACTCAAAAGAAATTGAAACAACTGCC
ACTGCTGAAGGAGGGGAAGTAGTTTTTCAAAATGCAGCTTTTACAATGTG
GAAACTGACATACCTGGAGCATAGACTAATGCCAATTTTGGATCAAAATT
TTATCGAATATAAAATAACAGTGAATGAAGATAAACCAATTTCAGAATCA
CATGTAAGAGAACTCATTGCTGAATTGCGGTGGCAATACAACAAATTTGC
AGTAATTACACATGGTAAAGGTCACTACAGAGTTGTAAAATATTCATCAG
TTGCGAATCATGCAGATAGAGTTTACGCTACTTTCAAGAGCAATAATAAG
AATGGGAATGTGATAGAGTTTAATCTACTTGATCAAAGAATAATATGGCA
GAACTGGTATGCGTTTACATCCTCAATGAAACAAGGCAACACTCTTGAAA
TATGCAAGAAACTACTGTTCCAAAAGATGAAGCGAGAAAGTAATCCGTTT
AAGGGACTGTCAACTGATAGAAAGATGGATGAGGTCTCTCAAATAGGAAT
T

Accordingly, preferably the Bovine RV NS2 polypeptide is encoded by the DNA nucleotide sequence substantially as set out in SEQ ID NO: 320, or a variant or fragment thereof.

One embodiment of codon optimised nucleic acid (DNA) encoding the Bovine RV NS2 polypeptide is provided herein as SEQ ID No: 321, as follows:

[SEQ ID No: 321]
ATGGCCGAGCTGGCCTGCTTTTGTTACCCTCACCTGGAAAGCGATACCTA
CCGGTTCATCCCCTTCAACAGCCTGGCCATCAAGTGCATGCTGACCGCCA
AGGTGGACAAGAAGGACCAGGACAAGTTCTACAACAGCATCATCTACGGA
ATCGCCCCTCCACCTCAGTTCAAGAAGCGGTACAACACCAACGACAACAG
CCGGGGCATGAACTACGAGACACCCATGTTCAACAAGGTGGCCGTGCTGA
TCTGCGAGGCCCTGAACTCCATCAAAGTGACCCAGTCCGATGTGGCCAGC
GTGCTGAGCAAAGTGATCTCTGTGCGGCACCTCGAGAACCTGGTGCTGCG
GAGAGAAAACCACCAGGACGTGCTGTTCCACAGCAAAGAGCTGCTGCTGA
GATCTGTGCTGATCGCCATCGGCCACTCCAAAGAGATCGAGACAACCGCC
ACAGCCGAAGGCGGAGAGGTGGTGTTTCAGAATGCCGCCTTCACCATGTG
GAAGCTGACCTACCTGGAACACCGGCTGATGCCCATCCTGGACCAGAACT
TCATCGAGTACAAGATCACCGTGAACGAGGACAAGCCCATCAGCGAGTCT
CACGTGCGGGAACTGATTGCCGAGCTGCGGTGGCAGTACAACAAGTTCGC
CGTGATCACACACGGCAAGGGCCACTACAGAGTGGTCAAGTACAGCAGCG
TGGCCAACCACGCCGATAGAGTGTACGCCACCTTCAAGAGCAACAACAAG
AACGGCAACGTGATCGAGTTCAACCTGCTGGACCAGCGGATCATCTGGCA
GAATTGGTACGCCTTTACCAGCAGCATGAAGCAGGGCAACACCCTGGAAA
TCTGCAAGAAGCTCCTGTTCCAGAAGATGAAGAGAGAGAGCAACCCCTTC
AAGGGCCTGAGCACCGACCGGAAGATGGATGAGGTGTCCCAGATCGGCAT
C

Hence, preferably the RNA construct comprises a sequence substantially as set out in SEQ ID No: 321, or a fragment or variant thereof.

In an embodiment, the RNA sequence corresponding to the codon optimised DNA sequence of SEQ ID No: 321 is provided herein as SEQ ID No: 322, as follows:

[SEQ ID No: 322]
AUGGCCGAGCUGGCCUGCUUUUGUUACCCUCACCUGGAAAGCGAUACCUA
CCGGUUCAUCCCCUUCAACAGCCUGGCCAUCAAGUGCAUGCUGACCGCCA
AGGUGGACAAGAAGGACCAGGACAAGUUCUACAACAGCAUCAUCUACGGA
AUCGCCCCUCCACCUCAGUUCAAGAAGCGGUACAACACCAACGACAACAG
CCGGGGCAUGAACUACGAGACACCCAUGUUCAACAAGGUGGCCGUGCUGA
UCUGCGAGGCCCUGAACUCCAUCAAAGUGACCCAGUCCGAUGUGGCCAGC
GUGCUGAGCAAAGUGAUCUCUGUGCGGCACCUCGAGAACCUGGUGCUGCG
GAGAGAAAACCACCAGGACGUGCUGUUCCACAGCAAAGAGCUGCUGCUGA
GAUCUGUGCUGAUCGCCAUCGGCCACUCCAAAGAGAUCGAGACAACCGCC
ACAGCCGAAGGCGGAGAGGUGGUGUUUCAGAAUGCCGCCUUCACCAUGUG
GAAGCUGACCUACCUGGAACACCGGCUGAUGCCCAUCCUGGACCAGAACU
UCAUCGAGUACAAGAUCACCGUGAACGAGGACAAGCCCAUCAGCGAGUCU
CACGUGCGGGAACUGAUUGCCGAGCUGCGGUGGCAGUACAACAAGUUCGC
CGUGAUCACACACGGCAAGGGCCACUACAGAGUGGUCAAGUACAGCAGCG
UGGCCAACCACGCCGAUAGAGUGUACGCCACCUUCAAGAGCAACAACAAG
AACGGCAACGUGAUCGAGUUCAACCUGCUGGACCAGCGGAUCAUCUGGCA
GAAUUGGUACGCCUUUACCAGCAGCAUGAAGCAGGGCAACACCCUGGAAA
UCUGCAAGAAGCUCCUGUUCCAGAAGAUGAAGAGAGAGAGCAACCCCUUC
AAGGGCCUGAGCACCGACCGGAAGAUGGAUGAGGUGUCCCAGAUCGGCAU
C

Hence, preferably the RNA construct comprises a sequence substantially as set out in SEQ ID No: 322, or a fragment or variant thereof.

In one embodiment, the at least one IIP is PBoV NP1 (D7RF52; Non-structural protein NP-1 Porcine bocavirus 1), or an orthologue thereof. One embodiment of the polypeptide sequence of PBoV NP1 is represented herein as SEQ ID No: 323, as follows:

[SEQ ID No: 323]
MSSARSDTDTGRRGKRSRSRSRSRDRDQAPGLPPKKRDYRRRSGERGSES
SPDRSTRGSPSCSTASRTSRVTSATWRRPNDSRDGGKIWGNKNKKNKTNP
YEVFSQHMARFKPDKSYCGFYWHSCRMARKGTDYIFTEGMRDFQKRCKDN
KCEWKDVREIMFGLKKVLDQGYRNMMYHFRHTQCEKCNYWDEVYKMHLAN
VSPSETEPQELTDEEILAAAMEVDGTHE

Therefore, preferably the RNA construct comprises a nucleotide sequence which encodes an amino acid sequence substantially as set out in SEQ ID No: 323, or a variant or fragment thereof.

In one embodiment, the PBoV NP1 polypeptide is encoded by the DNA nucleotide sequence of SEQ ID No: 324, as follows:

[SEQ ID No: 324]
ATGAGCTCAGCGAGATCCGACACAGACACAGGCAGGAGAGGGAAGCGGTC
GAGGAGCCGGTCGAGGAGCCGCGACCGGGACCAAGCACCGGGGCTGCCTC
CGAAAAAAAGGGACTATCGCCGTCGGAGTGGGGAGAGAGGCTCGGAGTCC
TCACCAGATCGCTCGACGAGGGGGAGCCCATCGTGCTCCACTGCTTCGAG
AACATCCCGAGTGACTTCAGCGACATGGAGGAGACCGAATGACTCTCGGG
ACGGGGGGAAAATATGGGGAAATAAAAATAAAAAGAATAAAACAAACCCT
TACGAGGTATTCAGCCAGCACATGGCCAGGTTCAAGCCAGATAAAAGCTA
TTGTGGCTTCTACTGGCACAGCTGCCGGATGGCTCGTAAGGGCACAGATT
ATATCTTTACCGAGGGAATGAGGGATTTCCAAAAACGCTGTAAAGACAAT
AAATGTGAGTGGAAAGATGTCAGGGAGATCATGTTCGGCCTCAAAAAGGT
CTTAGATCAGGGATATAGAAATATGATGTATCACTTTAGACATACCCAGT
GTGAGAAATGTAACTACTGGGATGAAGTGTATAAAATGCACCTGGCTAAC
GTGTCTCCTTCTGAAACAGAACCGCAGGAACTGACAGACGAGGAGATATT
AGCCGCGGCCATGGAGGTCGATGGCACCCACGAA

Accordingly, preferably the PBoV NP1 polypeptide is encoded by the DNA nucleotide sequence substantially as set out in SEQ ID NO: 324, or a variant or fragment thereof.

One embodiment of codon optimised nucleic acid (DNA) encoding the PBoV NP1 polypeptide is provided herein as SEQ ID No: 325, as follows:

[SEQ ID No: 325]
ATGAGCAGCGCCAGAAGCGATACCGACACAGGCAGACGGGGCAAGAGAAG
CAGAAGCCGGTCCAGAAGCAGAGACAGAGATCAGGCTCCTGGCCTGCCTC
CTAAGAAGCGGGACTACAGAAGAAGATCCGGCGAGAGAGGCAGCGAGAGC
AGCCCTGATAGAAGCACAAGAGGCAGCCCTAGCTGTAGCACCGCCAGCAG
AACAAGCAGAGTGACCTCTGCCACTTGGCGGAGGCCCAACGATTCTAGAG
ATGGCGGCAAGATCTGGGGCAACAAGAACAAGAAGAACAAAACGAACCCC
TACGAGGTGTTCAGCCAGCACATGGCCAGATTCAAGCCCGACAAGAGCTA
CTGCGGCTTCTACTGGCACAGCTGCCGGATGGCCAGAAAGGGCACCGACT
ACATCTTCACCGAGGGCATGAGAGACTTCCAGAAGCGGTGCAAGGACAAC
AAGTGCGAGTGGAAGGACGTGCGCGAGATCATGTTCGGCCTGAAGAAGGT
GCTGGACCAGGGCTACAGAAACATGATGTACCACTTCCGGCACACCCAGT
GCGAGAAGTGCAACTACTGGGACGAAGTGTACAAGATGCACCTGGCCAAC
GTGTCCCCATCCGAGACAGAGCCTCAAGAGCTGACCGACGAGGAAATTCT
GGCCGCTGCCATGGAAGTGGATGGCACACATGAA

Hence, preferably the RNA construct is encoded by a DNA sequence substantially as set out in SEQ ID No: 325, or a fragment or variant thereof.

In an embodiment, the RNA sequence corresponding to the codon optimised DNA sequence of SEQ ID No: 325 is provided herein as SEQ ID No: 326, as follows:

[SEQ ID No: 326]
AUGAGCAGCGCCAGAAGCGAUACCGACACAGGCAGACGGGGCAAGAGAAG
CAGAAGCCGGUCCAGAAGCAGAGACAGAGAUCAGGCUCCUGGCCUGCCUC
CUAAGAAGCGGGACUACAGAAGAAGAUCCGGCGAGAGAGGCAGCGAGAGC
AGCCCUGAUAGAAGCACAAGAGGCAGCCCUAGCUGUAGCACCGCCAGCAG
AACAAGCAGAGUGACCUCUGCCACUUGGCGGAGGCCCAACGAUUCUAGAG
AUGGCGGCAAGAUCUGGGGCAACAAGAACAAGAAGAACAAAACGAACCCC
UACGAGGUGUUCAGCCAGCACAUGGCCAGAUUCAAGCCCGACAAGAGCUA
CUGCGGCUUCUACUGGCACAGCUGCCGGAUGGCCAGAAAGGGCACCGACU
ACAUCUUCACCGAGGGCAUGAGAGACUUCCAGAAGCGGUGCAAGGACAAC
AAGUGCGAGUGGAAGGACGUGCGCGAGAUCAUGUUCGGCCUGAAGAAGGU
GCUGGACCAGGGCUACAGAAACAUGAUGUACCACUUCCGGCACACCCAGU
GCGAGAAGUGCAACUACUGGGACGAAGUGUACAAGAUGCACCUGGCCAAC
GUGUCCCCAUCCGAGACAGAGCCUCAAGAGCUGACCGACGAGGAAAUUCU
GGCCGCUGCCAUGGAAGUGGAUGGCACACAUGAA

Hence, preferably the RNA construct comprises a sequence substantially as set out in SEQ ID No: 326, or a fragment or variant thereof.

In one embodiment, the at least one IIP is Hepatitis E Orf3 (P69616; Protein ORF3 Hepatitis E virus genotype 1), or an orthologue thereof. One embodiment of the polypeptide sequence of Hepatitis E Orf3 is represented herein as SEQ ID No: 327, as follows:

[SEQ ID No: 327]
MGSRPCALGLFCCCSSCFCLCCPRHRPVSRLAAVVGGAAAVPAVVSGVTG
LILSPSQSPIFIQPTPSPPMSPLRPGLDLVFANPPDHSAPLGVTRPSAPP
LPHVVDLPQLGPRR

Therefore, preferably the RNA construct comprises a nucleotide sequence which encodes an amino acid sequence substantially as set out in SEQ ID No: 327, or a variant or fragment thereof.

In one embodiment, the Hepatitis E Orf3 polypeptide is encoded by the DNA nucleotide sequence of SEQ ID No: 328, as follows:

[SEQ ID No: 328]
ATGGGTTCGCGACCATGCGCCCTCGGCCTATTTTGTTGCTGCTCCTCATG
TTTTTGCCTATGCTGCCCGCGCCACCGCCCGGTCAGCCGTCTGGCCGCCG
TCGTGGGCGGCGCAGCGGCGGTTCCGGCGGTGGTTTCTGGGGTGACCGGG
TTGATTCTCAGCCCTTCGCAATCCCCTATATTCATCCAACCAACCCCTTC
GCCCCCGATGTCACCGCTGCGGCCGGGGCTGGACCTCGTGTTCGCCAACC
CGCCCGACCACTCGGCTCCGCTTGGCGTGACCAGGCCCAGCGCCCCGCCG
TTGCCTCACGTCGTAGACCTACCACAGCTGGGGCCGCGCCGC

Accordingly, preferably the Hepatitis E Orf3 polypeptide is encoded by the DNA nucleotide sequence substantially as set out in SEQ ID NO: 328, or a variant or fragment thereof.

One embodiment of codon optimised nucleic acid (DNA) encoding the Hepatitis E Orf3 polypeptide is provided herein as SEQ ID No: 329, as follows:

[SEQ ID No: 329]
ATGGGCAGCAGACCTTGTGCTCTGGGCCTGTTCTGCTGCTGCAGCTCCTG
CTTCTGCCTGTGCTGCCCTAGACACAGACCCGTGTCTAGACTGGCCGCTG
TTGTTGGCGGAGCTGCTGCTGTTCCAGCTGTGGTGTCTGGCGTGACAGGC
CTGATTCTGAGCCCTTCTCAGAGCCCCATCTTCATCCAGCCTACACCTAG
TCCTCCAATGAGCCCTCTGAGGCCTGGACTGGATCTGGTGTTCGCCAATC
CTCCTGATCACTCTGCCCCTCTGGGCGTGACAAGACCTTCTGCTCCTCCT
CTGCCACACGTGGTGGATCTGCCTCAACTGGGCCCTAGAAGA

Hence, preferably the RNA construct is encoded by a DNA sequence substantially as set out in SEQ ID No: 329, or a fragment or variant thereof.

In an embodiment, the RNA sequence corresponding to the codon optimised DNA sequence of SEQ ID No: 329 is provided herein as SEQ ID No: 330, as follows:

[SEQ ID No: 330]
AUGGGCAGCAGACCUUGUGCUCUGGGCCUGUUCUGCUGCUGCAGCUCCUG
CUUCUGCCUGUGCUGCCCUAGACACAGACCCGUGUCUAGACUGGCCGCUG
UUGUUGGCGGAGCUGCUGCUGUUCCAGCUGUGGUGUCUGGCGUGACAGGC
CUGAUUCUGAGCCCUUCUCAGAGCCCCAUCUUCAUCCAGCCUACACCUAG
UCCUCCAAUGAGCCCUCUGAGGCCUGGACUGGAUCUGGUGUUCGCCAAUC
CUCCUGAUCACUCUGCCCCUCUGGGCGUGACAAGACCUUCUGCUCCUCCU
CUGCCACACGUGGUGGAUCUGCCUCAACUGGGCCCUAGAAGA

Hence, preferably the RNA construct comprises a sequence substantially as set out in SEQ ID No: 330, or a fragment or variant thereof.

In one embodiment, the at least one IIP is Rotavirus NSP1 (Q99FX5; Non-structural protein 1 Rotavirus A (strain RVA/SA11-4F/G3P6[1])), or an orthologue thereof. Barro M, Patton J T (2007) Rotavirus NSP1 inhibits expression of type 1 interferon by antagonising the function of interferon regulatory factors IRF3, IRF5 and IRF7. J Virol, 81, 9, 4473-4481. This IIP is believed to induce degradation of IRF7. One embodiment of the polypeptide sequence of Rotavirus NSP1 is represented herein as SEQ ID No: 331, as follows:

[SEQ ID No: 331]
MATFKDACFHYRRLTALNRRLCNIGANSICMPVPDAKIKGWCLECCQIAD
LTHCYGCSLPHVCKWCVQNRRCFLDNEPHLLKLRTVKHPITKDKLQCIID
LYNIIFPINDKVIRKFERMIKQRKCRNQYKIEWYNHLLLPITLNAAAFKF
DENNLYYVFGLYEKSVSDIYAPYRIVNFINEFDKLLLDDINFTRMSNLPI
ELRNHYAKKYFQLSRLPSSKLKQIYFSDFTKETVIFNTYTKTPGRSIYRN
VTEFNWRDELELYSDLKNDKNKLIAAMMTSKYTRFYAHDNNFGRLKMTIF
ELGHHCQPNYVASNHPGNASDIQYCKWCNIKYFLSKIDWRIRDMYNLLME
FIKDCYKSNVNVGHCSSVENIYPLIKRLIWSLFTNHMDQTIEEVFNHMSP
VSVEGTNVIMLILGLNISLYNEIKRTLNVDSIPMVLNLNEFSSIVKSISS
KWYNVDELDKLPMSIKSTEELIEMKNSGTLTEEFELLISNSEDDNE

Therefore, preferably the RNA construct comprises a nucleotide sequence which encodes an amino acid sequence substantially as set out in SEQ ID No: 331, or a variant or fragment thereof.

In one embodiment, the Rotavirus NSP1 polypeptide is encoded by the DNA nucleotide sequence of SEQ ID No: 332, as follows:

[SEQ ID No: 332]
ATGGCTACTTTTAAAGATGCATGCTTTCATTATCGTAGATTAACTGCTTT
AAATCGGAGATTATGCAACATTGGTGCAAATTCTATTTGCATGCCAGTTC
CTGATGCGAAGATTAAGGGGTGGTGTTTAGAATGTTGTCAAATAGCTGAT
TTAACCCATTGTTATGGTTGCTCATTGCCGCATGTTTGCAAATGGTGTGT
TCAGAACAGAAGATGCTTCCTTGACAATGAACCTCATTTGCTTAAGCTTA
GAACTGTGAAACATCCAATTACCAAAGACAAATTACAGTGTATCATAGAC
TTGTACAATATAATATTTCCAATTAATGATAAAGTAATTAGAAAATTTGA
AAGAATGATAAAGCAAAGAAAATGTAGGAATCAATATAAAATTGAATGGT
ATAATCATTTGCTGCTCCCAATTACATTAAATGCTGCTGCATTTAAGTTT
GATGAAAATAATCTTTATTATGTTTTTGGGTTATATGAGAAATCAGTCAG
TGATATATATGCTCCATATAGAATTGTTAACTTTATAAATGAATTTGATA
AATTATTGCTTGATGATATTAACTTTACAAGAATGTCCAATCTACCAATA
GAGTTGAGAAACCATTATGCAAAGAAATACTTCCAATTATCAAGACTGCC
ATCATCAAAACTAAAGCAAATTTACTTTTCAGATTTTACTAAAGAAACTG
TGATTTTTAATACTTATACAAAAACGCCAGGAAGATCAATATACAGAAAT
GTAACTGAATTTAATTGGAGAGATGAATTGGAGCTTTATTCTGATTTAAA
AAATGATAAGAATAAATTAATTGCTGCAATGATGACGAGTAAGTATACTC
GGTTCTATGCTCATGATAATAATTTTGGAAGGTTGAAAATGACAATATTT
GAGTTGGGACATCATTGTCAGCCTAACTACGTGGCATCTAATCACCCAGG
CAATGCTTCCGATATCCAGTACTGTAAATGGTGTAATATAAAATATTTTC
TTAGTAAAATTGATTGGCGGATTCGTGATATGTATAATTTATTGATGGAA
TTTATTAAGGATTGTTATAAAAGTAATGTTAACGTTGGACATTGTAGTTC
TGTTGAAAACATATATCCTTTAATTAAAAGATTAATTTGGAGTTTGTTTA
CTAATCACATGGATCAAACAATTGAAGAAGTGTTTAATCACATGTCGCCA
GTGTCAGTTGAAGGTACGAATGTCATCATGTTGATTCTTGGATTGAATAT
TAGTTTGTATAATGAAATTAAGCGCACTTTGAATGTAGATAGCATACCAA
TGGTACTTAATTTAAATGAATTCAGTGAGGAACTGATTGAAATGAAGAAT
TCTGGAACTTTAACTGAAGAATTTGAGCTACTGATCTCCAACTCAGAAGA
TGACAATGAG

Accordingly, preferably the Rotavirus NSP1 polypeptide is encoded by the DNA nucleotide sequence substantially as set out in SEQ ID NO: 332, or a variant or fragment thereof.

One embodiment of codon optimised nucleic acid (DNA) encoding the Rotavirus NSP1 polypeptide is provided herein as SEQ ID No: 333, as follows:

[SEQ ID No: 333]
ATGGCCACCTTCAAGGACGCCTGCTTCCACTACAGACGGCTGACAGCCCT
GAATCGGCGGCTGTGTAATATCGGCGCCAACAGCATCTGCATGCCCGTGC
CTGACGCCAAGATCAAAGGCTGGTGCCTGGAATGCTGCCAGATCGCCGAT
CTCACCCACTGCTACGGCTGTTCTCTGCCCCATGTGTGCAAGTGGTGCGT
GCAGAACAGACGGTGCTTCCTGGACAACGAGCCCCATCTGCTGAAGCTGA
GAACCGTGAAGCACCCCATCACCAAGGACAAGCTGCAGTGCATCATCGAC
CTGTACAACATCATCTTCCCCATCAACGACAAAGTGATCCGGAAGTTCGA
GCGGATGATCAAGCAGCGGAAGTGCCGGAACCAGTACAAGATCGAGTGGT
ACAATCATCTGCTGCTGCCCATCACACTGAACGCCGCTGCCTTCAAGTTC
GACGAGAACAACCTGTACTACGTGTTCGGCCTGTACGAGAAGTCCGTGTC
CGACATCTACGCCCCTTACCGGATCGTGAACTTCATCAACGAGTTCGATA
AGCTGCTGCTGGACGACATCAACTTCACCCGGATGAGCAACCTGCCTATC
GAGCTGAGAAACCACTACGCCAAGAAGTACTTTCAGCTGAGCAGACTGCC
CAGCAGCAAGCTGAAGCAGATCTACTTCTCCGACTTCACCAAAGAAACCG
TGATCTTCAACACCTACACCAAGACACCCGGCAGATCCATCTACCGGAAC
GTGACCGAGTTCAACTGGCGGGACGAGCTGGAACTGTACAGCGACCTGAA
GAACGACAAGAACAAGCTGATCGCCGCCATGATGACCAGCAAGTACACCC
GGTTCTACGCCCACGACAACAATTTCGGCCGGCTGAAGATGACCATCTTC
GAGCTGGGCCACCACTGCCAGCCTAATTACGTGGCCTGGCGGATCCGGGA
CATGTACAACCTGCTGATGGAATTCATCAAGGACTGCTACAAGAGCAACG
TGAACGTGGGCCACTGCAGCAGCGTCGAGAACATCTACCCTCTGATCAAG
CGGCTGATCTGGTCCCTGTTCACCAACCACATGGACCAGACCATCGAAGA
GGTGTTCAATCACATGAGCCCCGTGTCCGTGGAAGGCACCAACGTGATCA
TGCTGATCCTGGGCCTGAACATCAGCCTGTACAATGAGATCAAGCGCACC
CTGAACGTGGACAGCATCCCCATGGTGCTGAACCTGAACGAGTTCAGCAG
CATCGTGAAGTCCATCTCCAGCAAGTGGTATAACGTGGACGAACTGGACA
AACTGCCCATGAGCATCAAGTCCACCGAGGAACTGATCGAGATGAAGAAC
AGCGGCACCCTGACCGAGGAATTCGAGCTGCTGATCTCCAACAGCGAGGA
CGACAACGAG

Hence, preferably the RNA construct is encoded by a DNA sequence substantially as set out in SEQ ID No: 333, or a fragment or variant thereof.

In an embodiment, the RNA sequence corresponding to the codon optimised DNA sequence of SEQ ID No: 333 is provided herein as SEQ ID No: 334, as follows:

[SEQ ID No: 334]
AUGGCCACCUUCAAGGACGCCUGCUUCCACUACAGACGGCUGACAGCCCU
GAAUCGGCGGCUGUGUAAUAUCGGCGCCAACAGCAUCUGCAUGCCCGUGC
CUGACGCCAAGAUCAAAGGCUGGUGCCUGGAAUGCUGCCAGAUCGCCGAU
CUCACCCACUGCUACGGCUGUUCUCUGCCCCAUGUGUGCAAGUGGUGCGU
GCAGAACAGACGGUGCUUCCUGGACAACGAGCCCCAUCUGCUGAAGCUGA
GAACCGUGAAGCACCCCAUCACCAAGGACAAGCUGCAGUGCAUCAUCGAC
CUGUACAACAUCAUCUUCCCCAUCAACGACAAAGUGAUCCGGAAGUUCGA
GCGGAUGAUCAAGCAGCGGAAGUGCCGGAACCAGUACAAGAUCGAGUGGU
ACAAUCAUCUGCUGCUGCCCAUCACACUGAACGCCGCUGCCUUCAAGUUC
GACGAGAACAACCUGUACUACGUGUUCGGCCUGUACGAGAAGUCCGUGUC
CGACAUCUACGCCCCUUACCGGAUCGUGAACUUCAUCAACGAGUUCGAUA
AGCUGCUGCUGGACGACAUCAACUUCACCCGGAUGAGCAACCUGCCUAUC
GAGCUGAGAAACCACUACGCCAAGAAGUACUUUCAGCUGAGCAGACUGCC
CAGCAGCAAGCUGAAGCAGAUCUACUUCUCCGACUUCACCAAAGAAACCG
UGAUCUUCAACACCUACACCAAGACACCCGGCAGAUCCAUCUACCGGAAC
GUGACCGAGUUCAACUGGCGGGACGAGCUGGAACUGUACAGCGACCUGAA
GAACGACAAGAACAAGCUGAUCGCCGCCAUGAUGACCAGCAAGUACACCC
GGUUCUACGCCCACGACAACAAUUUCGGCCGGCUGAAGAUGACCAUCUUC
GAGCUGGGCCACCACUGCCAGCCUAAUUACGUGGCCUCUAAUCACCCCGG
CAACGCCAGCGAUAUCCAGUACUGCAAAUGGUGCAAUAUCAAGUACUUCC
UGAGCAAGAUCGACUGGCGGAUCCGGGACAUGUACAACCUGCUGAUGGAA
UUCAUCAAGGACUGCUACAAGAGCAACGUGAACGUGGGCCACUGCAGCAG
CGUCGAGAACAUCUACCCUCUGAUCAAGCGGCUGAUCUGGUCCCUGUUCA
CCAACCACAUGGACCAGACCAUCGAAGAGGUGUUCAAUCACAUGAGCCCC
GUGUCCGUGGAAGGCACCAACGUGAUCAUGCUGAUCCUGGGCCUGAACAU
CAGCCUGUACAAUGAGAUCAAGCGCACCCUGAACGUGGACAGCAUCCCCA
UGGUGCUGAACCUGAACGAGUUCAGCAGCAUCGUGAAGUCCAUCUCCAGC
AAGUGGUAUAACGUGGACGAACUGGACAAACUGCCCAUGAGCAUCAAGUC
CACCGAGGAACUGAUCGAGAUGAAGAACAGCGGCACCCUGACCGAGGAAU
UCGAGCUGCUGAUCUCCAACAGCGAGGACGACAACGAG

Hence, preferably the RNA construct comprises a sequence substantially as set out in SEQ ID No: 334, or a fragment or variant thereof.

In one embodiment, the at least one IIP is KSHV ORF45 (Q77UV9; HHV8 ORF 45 Human herpesvirus 8 OX), or an orthologue thereof. One embodiment of the polypeptide sequence of KSHV ORF45 is represented herein as SEQ ID No: 335, as follows:

[SEQ ID No: 335]
MAMFVRTSSSTHDEERMLPIEGAPRRRPPVKFIFPPPPLSSLPGFGRPRG
YAGPTVIDMSAPDDVFAEDTPSPPATPLDLQISPDQSSGESEYDEDEEDE
DEEENDDVQEEDEPEGYPADFFQPLSHLRPRPLARRAHTPKPVAVVAGRV
RSSTDTAESEASMGWVSQDDGFSPAGLSPSDDEGVAILEPMAAYTGTGAY
GLSPASRNSVPGTQSSPYSDPDEGPSWRPLRAAPTAIVDLTSDSDSDDSS
NSPDVNNEAAFTDARHFSHQPPSSEEDGEDQGEVLSQRIGLMDVGQKRKR
QSTASSGSEDVVRCQRQPNLSRKAVASVIIISSGSDTDEEPSSAVSVIVS
PSSTKGHLPTQSPSTSAHSISSGSTTTAGSRCSDPTRILASTPPLCGNGA
YNWPWLD

Therefore, preferably the RNA construct comprises a nucleotide sequence which encodes an amino acid sequence substantially as set out in SEQ ID No: 335, or a variant or fragment thereof.

In one embodiment, the KSHV ORF45 polypeptide is encoded by the DNA nucleotide sequence of SEQ ID No: 336, as follows:

[SEQ ID No: 336]
CGACCCCCCGTGAAGTTCATATTCCCACCTCCACCTCTTTCATCACTTCC
AGGATTTGGCAGGCCGCGCGGCTATGCTGGACCCACGGTGATAGATATGT
CTGCCCCAGACGACGTCTTCGCCGAGGACACGCCATCGCCGCCAGCAACC
CCTCTGGATCTACAGATATCCCCGGATCAGTCGAGCGGCGAATCTGAATA
TGACGAGGATGAGGAAGATGAAGATGAAGAAGAAAATGACGATGTTCAGG
AGGAAGACGAGCCAGAGGGGTACCCTGCAGACTTTTTTCAACCTTTATCT
CACTTGCGCCCGAGGCCTCTGGCCAGACGGGCCCATACGCCCAAACCGGT
AGCAGTGGTAGCGGGCCGCGTGCGCAGTTCAACGGACACGGCGGAGTCCG
AGGCGTCCATGGGATGGGTTAGTCAGGATGACGGATTTTCCCCTGCTGGG
CTCTCACCTTCAGACGACGAGGGGGTTGCTATCCTGGAACCGATGGCGGC
ATACACTGGGACCGGGGCATACGGACTTTCACCTGCTTCCAGAAATAGTG
TACCTGGAACACAAAGTTCACCATACAGCGACCCTGATGAAGGGCCCTCG
TGGCGCCCCCTGCGCGCCGCACCCACCGCGATCGTCGACCTGACATCGGA
CTCTGATAGCGATGACAGTTCCAACTCTCCGGACGTGAACAATGAGGCCG
CGTTTACCGACGCGCGCCATTTTTCCCACCAGCCACCCTCGTCCGAGGAG
GACGGAGAAGACCAAGGGGAAGTATTGAGTCAGAGAATCGGGCTCATGGA
CGTGGGCCAGAAGCGCAAAAGGCAGTCTACCGCCTCCTCIGGTAGCGAGG
ATGTGGTGCGCTGCCAGAGACAACCAAACTTAAGCCGCAAAGCAGTGGCG
TCTGTGATAATTATATCCTCGGGGAGTGACACAGACGAGGAGCCCTCGTC
CGCCGTGAGCGTGATCGTGTCTCCGTCGAGCACAAAGGGTCACCTCCCAA
CCCAATCTCCCAGTACTTCCGCCCACTCGATTTCATCAGGAAGCACAACT
ACCGCGGGGTCCAGGTGCAGCGACCCAACCCGCATCCTGGCCTCCACGCC
ACCCCTGTGTGGAAACGGTGCATATAACTGGCCGTGGCTGGAC

Accordingly, preferably the KSHV ORF45 polypeptide is encoded by the DNA nucleotide sequence substantially as set out in SEQ ID NO: 336, or a variant or fragment thereof.

One embodiment of codon optimised nucleic acid (DNA) encoding the KSHV ORF45 polypeptide is provided herein as SEQ ID No: 337, as follows:

[SEQ ID No: 337]
ATGGCCATGTTTGTGCGGACCAGCAGCAGCACCCACGACGAGGAAAGAAT
GCTGCCTATCGAGGGCGCTCCTCGGAGAAGGCCTCCTGTGAAGTTCATCT
TCCCACCTCCACCACTGAGCAGCCTGCCTGGATTTGGCAGACCTAGAGGC
TACGCCGGACCTACCGTGATCGATATGAGCGCCCCTGACGATGTGTTCGC
CGAGGATACACCTTCTCCACCAGCCACACCTCTGGACCTGCAGATCAGCC
CTGATCAGTCTAGCGGCGAGAGCGAGTACGATGAGGACGAAGAGGACGAG
GATGAGGAAGAGAACGACGACGTCCAAGAGGAAGATGAGCCCGAGGGCTA
CCCCGCCGATTTCTTTCAGCCTCTGTCTCACCTGAGGCCTGCCGAGTCCG
AAGCCAGCATGGGATGGGTGTCACAGGACGATGGATTCAGCCCTGCCGGA
CTGAGCCCTTCCGATGATGAAGGCGTGGCCATCCTGGAACCTATGGCCGC
CTATACTGGCACAGGCGCCTATGGACTGTCTCCCGCCAGCAGAAATAGCG
TGCCAGGCACACAGAGCAGCCCCTACTCTGATCCTGATGAGGGCCCATCT
TGGAGGCCCCTTAGAGCTGCTCCTACCGCCATCGTGGATCTGACCAGCGA
CAGCGATAGCGACGACAGCAGCAATAGCCCCGACGTGAACAATGAGGCCG
CCTTCACAGACGCCCGGCACTTTTCTCATCAGCCTCCAAGCAGCGAAGAG
GATGGCGAGGATCAGGGCGAAGTGCTGTCTCAGAGAATCGGCCTGATGGA
CGTGGGCCAGAAGCGGAAGAGACAGAGCACAGCCAGCAGCGGCTCTGAGG
ATGTCGTCAGATGCCAGAGACAGCCCAACCTGAGCAGAAAGGCCGTGGCC
AGCGTGATCATCATCAGCTCTGGCAGCGACACCGATGAGGAACCTAGCTC
TGCCGTGTCCGTGATCGTGTCTCCTAGCAGCACCAAGGGCCATCTGCCTA
CACAGAGCCCTAGCACAAGCGCCCACTCTATCTCTAGCGGCAGCACAACA
ACAGCCGGCAGCAGATGCAGCGACCCCACAAGAATTCTGGCCAGCACACC
TCCTCTGTGCGGCAACGGCGCTTACAATTGGCCTTGGCTGGAT

Hence, preferably the RNA construct is encoded by a DNA sequence substantially as set out in SEQ ID No: 337, or a fragment or variant thereof.

In an embodiment, the RNA sequence corresponding to the codon optimised DNA sequence of SEQ ID No: 337 is provided herein as SEQ ID No: 338, as follows:

[SEQ ID No: 338]
AUGGCCAUGUUUGUGCGGACCAGCAGCAGCACCCACGACGAGGAAAGAAU
GCUGCCUAUCGAGGGCGCUCCUCGGAGAAGGCCUCCUGUGAAGUUCAUCU
UCCCACCUCCACCACUGAGCAGCCUGCCUGGAUUUGGCAGACCUAGAGGC
UACGCCGGACCUACCGUGAUCGAUAUGAGCGCCCCUGACGAUGUGUUCGC
CGAGGAUACACCUUCUCCACCAGCCACACCUCUGGACCUGCAGAUCAGCC
CUGAUCAGUCUAGCGGCGAGAGCGAGUACGAUGAGGACGAAGAGGACGAG
GAUGAGGAAGAGAACGACGACGUCCAAGAGGAAGAUGAGCCCGAGGGCUA
CCCCGCCGAUUUCUUUCAGCCUCUGUCUCACCUGAGGCCUCGGCCUCUUG
CUAGAAGGGCCCACACACCUAAACCUGUGGCUGUGGUGGCCGGAAGAGUG
CGGUCUAGCACAGAUACAGCCGAGUCCGAAGCCAGCAUGGGAUGGGUGUC
ACAGGACGAUGGAUUCAGCCCUGCCGGACUGAGCCCUUCCGAUGAUGAAG
GCGUGGCCAUCCUGGAACCUAUGGCCGCCUAUACUGGCACAGGCGCCUAU
GGACUGUCUCCCGCCAGCAGAAAUAGCGUGCCAGGCACACAGAGCAGCCC
CUACUCUGAUCCUGAUGAGGGCCCAUCUUGGAGGCCCCUUAGAGCUGCUC
CUACCGCCAUCGUGGAUCUGACCAGCGACAGCGAUAGCGACGACAGCAGC
AAUAGCCCCGACGUGAACAAUGAGGCCGCCUUCACAGACGCCCGGCACUU
UUCUCAUCAGCCUCCAAGCAGCGAAGAGGAUGGCGAGGAUCAGGGCGAAG
UGCUGUCUCAGAGAAUCGGCCUGAUGGACGUGGGCCAGAAGCGGAAGAGA
CAGAGCACAGCCAGCAGCGGCUCUGAGGAUGUCGUCAGAUGCCAGAGACA
GCCCAACCUGAGCAGAAAGGCCGUGGCCAGCGUGAUCAUCAUCAGCUCUG
GCAGCGACACCGAUGAGGAACCUAGCUCUGCCGUGUCCGUGAUCGUGUCU
CCUAGCAGCACCAAGGGCCAUCUGCCUACACAGAGCCCUAGCACAAGCGC
CCACUCUAUCUCUAGCGGCAGCACAACAACAGCCGGCAGCAGAUGCAGCG
ACCCCACAAGAAUUCUGGCCAGCACACCUCCUCUGUGCGGCAACGGCGCU
UACAAUUGGCCUUGGCUGGAU

Hence, preferably the RNA construct comprises a sequence substantially as set out in SEQ ID No: 338, or a fragment or variant thereof.

In one embodiment, the at least one IIP is EBV BZLF-1 (P03206; Trans-activator protein BZLF1 Epstein-Barr virus (strain B95-8)), or an orthologue thereof. Hahn A M, Huye L E, Ning S, Webster-Cyriaque J M Pagano J S (2005) Interferon Regulatory Factor 7 Is Negatively Regulated by the Epstein-Barr Virus Immediate-Early Gene, J Virol, 79, 15, 10040-10052 doi:10.1128/JVI.790.15.10040-10052.2005. One embodiment of the polypeptide sequence of EBV BZLF-1 is represented herein as SEQ ID No: 339, as follows:

[SEQ ID No: 339]
MMDPNSTSEDVKFTPDPYQVPFVQAFDQATRVYQDLGGPSQAPLPCVLWP
VLPEPLPQGQLTAYHVSTAPTGSWFSAPQPAPENAYQAYAAPQLFPVSDI
TQNQQTNQAGGEAPQPGDNSTVQTAAAVVFACPGANQGQQLADIGVPQPA
PVAAPARRTRKPQQPESLEECDSELEIKRYKNRVASRKCRAKFKQLLQHY
REVAAAKSSENDRLRLLLKQMCPSLDVDSIIPRTPDVLHEDLLNF

Therefore, preferably the RNA construct comprises a nucleotide sequence which encodes an amino acid sequence substantially as set out in SEQ ID No: 339, or a variant or fragment thereof.

In one embodiment, the EBV BZLF-1 polypeptide is encoded by the DNA nucleotide sequence of SEQ ID No: 340, as follows:

[SEQ ID No: 340]
ATGATGGACCCAAACTCGACTTCTGAAGATGTAAAATTTACACCTGACCC
ATACCAGGTGCCTTTTGTACAAGCTTTTGACCAAGCTACCAGAGTCTATC
AGGACCTGGGAGGGCCATCGCAAGCTCCTTTGCCTTGTGTGCTGTGGCCG
GTGCTGCCAGAGCCTCTGCCACAAGGCCAGCTAACTGCCTATCATGTTTC
AACCGCTCCGACTGGGTCGTGGTTTTCTGCCCCTCAGCCTGCTCCTGAGA
ATGCTTATCAAGCTTATGCAGCACCTCAGCTGTTCCCAGTCTCCGACATA
ACCCAGAATCAACAGACTAACCAAGCCGGGGGAGAAGCACCTCAACCTGG
AGACAATTCTACTGTTCAAACAGCAGCAGCAGTGGTGTTTGCTTGCCCCG
GGGCTAACCAAGGACAACAGCTAGCAGACATTGGTGTTCCACAGCCTGCA
CCAGTGGCTGCCCCGGCACGACGCACACGGAAACCACAACAGCCAGAATC
GCTGGAGGAATGCGATTCTGAACTAGAAATAAAGCGATACAAGAATCGGG
TGGCTTCCAGAAAATGCCGGGCCAAGTTTAAGCAACTGCTGCAGCACTAC
CGTGAGGTCGCTGCTGCCAAATCATCTGAAAATGACAGGCTGCGCCTCCT
GTTGAAGCAGATGTGCCCAAGCCTGGATGTTGACTCCATTATCCCCCGGA
CACCAGATGTTTTACACGAGGATCTCTTAAATTTC

Accordingly, preferably the EBV BZLF-1 polypeptide is encoded by the DNA nucleotide sequence substantially as set out in SEQ ID NO: 340, or a variant or fragment thereof.

One embodiment of codon optimised nucleic acid (DNA) encoding the EBV BZLF-1 polypeptide is provided herein as SEQ ID No: 341, as follows:

[SEQ ID No: 341]
ATGATGGACCCCAACAGCACCAGCGAGGACGTGAAGTTCACCCCTGATCC
TTACCAGGTGCCATTCGTGCAGGCCTTCCCTGAACCTCTGCCTCAGGGAC
AGCTGACAGCCTACCATGTGTCTACAGCCCCTACCGGCAGCTGGTTTTCT
GCTCCTCAACCTGCTCCTGAGAACGCCTACCAGGCCTATGCTGCCCCTCA
GCTGTTTCCCGTGTCCGACATCACCCAGAACCAGCAGACAAATCAGGCTG
GCGGAGAAGCTCCTCAGCCTGGCGATAATAGCACCGTGCAGACAGCTGCC
GCCGTGGTGTTTGCTTGTCCTGGCGCTAATCAGGGCCAGCAGCTGGCTGA
TATTGGCGTGCCACAACCAGCTCCAGTGGCCGCTCCTGCCAGAAGAACAA
GAAAGCCTCAGCAGCCCGAGAGCCTGGAAGAGTGCGATAGCGAGCTGGAA
ATCAAGCGGTACAAGAACAGAGTGGCCAGCCGGAAGTGCCGGGCCAAGTT
TAAACAGCTGCTCCAGCACTACAGAGAGGTGGCCGCTGCCAAGAGCAGCG
AGAACGATAGACTGCGGCTGCTGCTGAAGCAGATGTGCCCTAGCCTGGAC
GTGGACAGCATCATCCCCAGAACACCCGATGTGCTGCACGAGGACCTGCT
GAACTTT

Hence, preferably the RNA construct is encoded by a DNA sequence substantially as set out in SEQ ID No: 341, or a fragment or variant thereof.

In an embodiment, the RNA sequence corresponding to the codon optimised DNA sequence of SEQ ID No: 341 is provided herein as SEQ ID No: 342, as follows:

[SEQ ID No: 342]
AUGAUGGACCCCAACAGCACCAGCGAGGACGUGAAGUUCACCCCUGAUCC
UUACCAGGUGCCAUUCGUGCAGGCCUUCGAUCAGGCCACCAGAGUGUACC
AGGAUCUCGGCGGACCUUCUCAGGCUCCUCUGCCUUGUGUUCUGUGGCCU
GUGCUGCCUGAACCUCUGCCUCAGGGACAGCUGACAGCCUACCAUGUGUC
UACAGCCCCUACCGGCAGCUGGUUUUCUGCUCCUCAACCUGCUCCUGAGA
ACGCCUACCAGGCCUAUGCUGCCCCUCAGCUGUUUCCCGUGUCCGACAUC
ACCCAGAACCAGCAGACAAAUCAGGCUGGCGGAGAAGCUCCUCAGCCUGG
CGAUAAUAGCACCGUGCAGACAGCUGCCGCCGUGGUGUUUGCUUGUCCUG
GCGCUAAUCAGGGCCAGCAGCUGGCUGAUAUUGGCGUGCCACAACCAGCU
CCAGUGGCCGCUCCUGCCAGAAGAACAAGAAAGCCUCAGCAGCCCGAGAG
CCUGGAAGAGUGCGAUAGCGAGCUGGAAAUCAAGCGGUACAAGAACAGAG
UGGCCAGCCGGAAGUGCCGGGCCAAGUUUAAACAGCUGCUCCAGCACUAC
AGAGAGGUGGCCGCUGCCAAGAGCAGCGAGAACGAUAGACUGCGGCUGCU
GCUGAAGCAGAUGUGCCCUAGCCUGGACGUGGACAGCAUCAUCCCCAGAA
CACCCGAUGUGCUGCACGAGGACCUGCUGAACUUU

Hence, preferably the RNA construct comprises a sequence substantially as set out in SEQ ID No: 342, or a fragment or variant thereof.

In one embodiment, the at least one IIP is MuHV Orf73 (041974; MHV68 ORF73 protein Murine herpesvirus 4), or an orthologue thereof. One embodiment of the polypeptide sequence of MuHV Orf73 is represented herein as SEQ ID No: 343, as follows:

[SEQ ID No: 343]
MPTSPPTTRNTTSGKTRSGCKRRCFNKPAAMPPKRRRAPKRPAPPPPPGC
QGDEESSQGTQTPNPPSPPVPPSSPTLPSSPVPPSSPVHEPPSPSPPPAP
PSPDVDVEGLDVGETDDPGPPPPKRYSRYQKPHNPSDPLPKKYQGMRRHL
QVTAPRLFDPEGHPPTHFKSAVMFSSTHPYTLNKLHKCIQSKHVLSTPVS
CLPLVPGTTQQCVTYYLLSFVEDKKQAKKLKRVVLAYCEKYHSSVEGTIV
KAKPYFPLPEPPTEPPTDPEQPSTSTQASGTQHGPTASLDAGAEQGATGS
PGSSPGQQGQGSQT

Therefore, preferably the RNA construct comprises a nucleotide sequence which encodes an amino acid sequence substantially as set out in SEQ ID No: 343, or a variant or fragment thereof.

In one embodiment, the MuHV Orf73 polypeptide is encoded by the DNA nucleotide sequence of SEQ ID No: 344, as follows:

[SEQ ID No: 344]
ATGCCCACATCCCCACCGACTACACGCAACACAACCTCAGGCAAAACCAG
ATCAGGGTGCAAACGTAGGTGCTTCAACAAACCAGCAGCCATGCCTCCTA
AAAGACGCCGCGCTCCAAAAAGACCAGCCCCTCCTCCACCACCGGGATGC
CAAGGTGATGAGGAGTCCAGCCAGGGAACTCAAACGCCAAACCCCCCATC
ACCACCAGTGCCCCCTTCATCACCAACACTTCCCTCATCCCCCGTCCCTC
CTTCATCACCAGTACATGAGCCACCATCTCCTTCCCCCCCACCAGCCCCA
CCATCACCAGATGTTGATGTTGAAGGTTTAGATGTAGGAGAGACAGACGA
TCCCGGTCCCCCTCCACCAAAAAGATACTCCAGGTATCAAAAACCGCATA
ATCCATCTGATCCATTGCCTAAAAAATATCAGGGAATGCGAAGACACCTG
CAGGTGACAGCACCCAGGTTATTTGATCCCGAGGGTCACCCCCCAACACA
TTTTAAGTCAGCTGTTATGTTTAGTAGCACACATCCCTACACTTTGAATA
AACTTCACAAGTGTATCCAAAGCAAACATGTACTCTCAACACCAGTTAGC
TGTTTACCCTTGGTACCAGGCACAACACAACAGTGTGTAACATACTATTT
ACTTTCATTTGTTGAAGACAAGAAACAGGCCAAAAAACTAAAAAGGGTTG
TCTTGGCCTACTGTGAAAAATACCACAGCAGCGTAGAAGGTACTATAGTC
AAGGCAAAGCCTTATTTTCCCTTACCAGAGCCCCCTACAGAGCCCCCTAC
AGACCCCGAGCAGCCATCCACAAGTACACAAGCTTCTGGCACACAACATG
GTCCCACAGCATCTCTGGATGCCGGTGCAGAGCAAGGTGCCACAGGATCA
CCTGGATCTAGTCCAGGACAACAGGGACAAGGGTCTCAGACA

Accordingly, preferably the MuHV Orf73 polypeptide is encoded by the DNA nucleotide sequence substantially as set out in SEQ ID NO: 344, or a variant or fragment thereof.

One embodiment of codon optimised nucleic acid (DNA) encoding the MuHV Orf73 polypeptide is provided herein as SEQ ID No: 345, as follows:

[SEQ ID No: 345]
ATGCCTACAAGCCCTCCTACCACCAGAAACACCACCAGCGGCAAGACAAGAAGCGGCTGCAAGCGGCGGTGCTTCAAC
AAACCTGCTGCCATGCCTCCTAAGCGGCGGAGAGCACCTAAAAGACCTGCTCCTCCTCCACCTCCTGGTTGCCAAGGC
GACGAGGAATCTTCTCAGGGCACCCAGACACCTAATCCTCCATCTCCACCTGTGCCTCCAAGCAGCCCTACACTGCCA
TCTTCTCCAGTGCCACCTAGCAGCCCAGTGCACGAACCACCTAGTCCAAGTCCTCCACCAGCTCCACCTTCTCCAGAC
GTGGACGTGGAAGGACTGGATGTGGGCGAGACAGACGATCCTGGACCTCCACCACCTAAGCGGTACAGCAGATACCAG
AAGCCTCACAACCCCAGCGATCCTCTGCCTAAGAAATACCAGGGCATGCGCCGGCATCTGCAAGTGACAGCCCCTAGA
CTGTTCGACCCTGAGGGACACCCTCCTACACACTTCAAGAGCGCCGTGATGTTCAGCAGCACACACCCCTACACTCTG
AACAAGCTGCACAAGTGCATCCAGAGCAAACACGTGCTGAGCACCCCTGTGTCCTGTCTGCCTCTGGTGCCTGGAACC
ACACAGCAGTGCGTGACCTACTACCTGCTGAGCTTCGTGGAAGATAAGAAGCAGGCCAAGAAACTGAAGAGAGTGGTG
CTGGCCTACTGCGAGAAGTACCACAGCAGCGTGGAAGGCACCATCGTGAAGGCCAAGCCTTACTTCCCACTGCCTGAG
CCTCCAACCGAGCCACCTACAGATCCTGAGCAGCCTAGCACAAGCACACAGGCCTCTGGAACACAGCACGGACCTACA
GCTAGTCTGGATGCTGGTGCTGAACAGGGCGCCACAGGATCACCTGGAAGTAGCCCTGGACAGCAAGGCCAGGGATCT
CAGACA

Hence, preferably the RNA construct is encoded by a DNA sequence substantially as set out in SEQ ID No: 345, or a fragment or variant thereof.

In an embodiment, the RNA sequence corresponding to the codon optimised DNA sequence of SEQ ID No: 345 is provided herein as SEQ ID No: 346, as follows:

[SEQ ID No: 346]
AUGCCUACAAGCCCUCCUACCACCAGAAACACCACCAGCGGCAAGACAAGAAGCGGCUGCAAGCGGCGGUGCUUCAAC
AAACCUGCUGCCAUGCCUCCUAAGCGGCGGAGAGCACCUAAAAGACCUGCUCCUCCUCCACCUCCUGGUUGCCAAGGC
GACGAGGAAUCUUCUCAGGGCACCCAGACACCUAAUCCUCCAUCUCCACCUGUGCCUCCAAGCAGCCCUACACUGCCA
UCUUCUCCAGUGCCACCUAGCAGCCCAGUGCACGAACCACCUAGUCCAAGUCCUCCACCAGCUCCACCUUCUCCAGAC
GUGGACGUGGAAGGACUGGAUGUGGGCGAGACAGACGAUCCUGGACCUCCACCACCUAAGCGGUACAGCAGAUACCAG
AAGCCUCACAACCCCAGCGAUCCUCUGCCUAAGAAAUACCAGGGCAUGCGCCGGCAUCUGCAAGUGACAGCCCCUAGA
CUGUUCGACCCUGAGGGACACCCUCCUACACACUUCAAGAGCGCCGUGAUGUUCAGCAGCACACACCCCUACACUCUG
AACAAGCUGCACAAGUGCAUCCAGAGCAAACACGUGCUGAGCACCCCUGUGUCCUGUCUGCCUCUGGUGCCUGGAACC
ACACAGCAGUGCGUGACCUACUACCUGCUGAGCUUCGUGGAAGAUAAGAAGCAGGCCAAGAAACUGAAGAGAGUGGUG
CUGGCCUACUGCGAGAAGUACCACAGCAGCGUGGAAGGCACCAUCGUGAAGGCCAAGCCUUACUUCCCACUGCCUGAG
CCUCCAACCGAGCCACCUACAGAUCCUGAGCAGCCUAGCACAAGCACACAGGCCUCUGGAACACAGCACGGACCUACA
GCUAGUCUGGAUGCUGGUGCUGAACAGGGCGCCACAGGAUCACCUGGAAGUAGCCCUGGACAGCAAGGCCAGGGAUCU
CAGACA

Hence, preferably the RNA construct comprises a sequence substantially as set out in SEQ ID No: 346, or a fragment or variant thereof.

In one embodiment, the at least one IIP is Torque Teno virus Orf2 (A7XCD9; ORF2 protein Torque teno virus (isolate Human/Finland/Hel32/2002)), or an orthologue thereof. Zheng H, Ye L, Fang X, Li B, Wang Y, Xiang X, Kong L, Wang W et al. (2007) Torque teno virus (SANBAN isolate) ORF2 protein suppresses NF-kB pathways via interaction with IkappaB kinases. J Virol, 81, 21, 11917-11924. It is believed that this IIP suppresses canonical and non-canonical Nf-KB pathways. One embodiment of the polypeptide sequence of Torque Teno virus Orf2 is represented herein as SEQ ID No: 347, as follows:

[SEQ ID No: 347]
MWQPPTQNGTQLERHWFESVWRSHAAFCSCGDCIGHLQHLATNLGRPPAPQPPRDQHPPHIRGLPALPAPPSNRNSWP
GTGGDAAGGEAGGSRGAGDGGDGELADEDLLDAIALAAE

Therefore, preferably the RNA construct comprises a nucleotide sequence which encodes an amino acid sequence substantially as set out in SEQ ID No: 347, or a variant or fragment thereof.

In one embodiment, the Torque Teno virus Orf2 polypeptide is encoded by the DNA nucleotide sequence of SEQ ID No: 348, as follows:

[SEQ ID No: 348]
ATGTGGCAGCCACCTACCCAGAATGGAACCCAACTCGAACGGCACTGGTTCGAGTCCGTTTGGCGTTCGCATGCTGCC
TTTTGTAGCTGTGGCGACTGTATTGGCCATCTTCAGCATCTGGCTACTAACCTGGGTCGACCACCTGCTCCACAACCG
CCGCGAGACCAACACCCACCGCACATAAGAGGGCTCCCGGCACTCCCGGCACCTCCCAGTAACAGAAACTCATGGCCT
GGTACTGGTGGAGACGCCGCCGGAGGAGAGGCTGGTGGAAGCCGAGGCGCAGGAGATGGAGGAGACGGAGAGCTCGCA
GACGAGGACCTGCTAGACGCCATCGCGCTCGCCGCAGAG

Accordingly, preferably the Torque Teno virus Orf2 polypeptide is encoded by the DNA nucleotide sequence substantially as set out in SEQ ID NO: 348, or a variant or fragment thereof.

One embodiment of codon optimised nucleic acid (DNA) encoding the Torque Teno virus Orf2 polypeptide is provided herein as SEQ ID No: 349, as follows:

[SEQ ID No: 349]
ATGTGGCAGCCTCCTACACAGAATGGCACCCAGCTGGAACGGCATTGGTTCGAGAGCGTTTGGAGAAGCCACGCCGCT
TTCTGCAGCTGCGGAGATTGCATCGGACATCTGCAGCACCTGGCCACCAATCTGGGTAGACCTCCAGCTCCTCAGCCT
CCTCGAGATCAGCACCCTCCTCACATCAGAGGACTGCCTGCACTTCCTGCTCCTCCAAGCAACAGAAACAGCTGGCCT
GGCACAGGCGGAGATGCTGCTGGCGGAGAAGCTGGTGGATCTAGAGGTGCCGGCGACGGTGGCGACGGCGAACTTGCT
GATGAAGATCTGCTGGACGCTATCGCCCTGGCCGCTGAA

Hence, preferably the RNA construct is encoded by a DNA sequence substantially as set out in SEQ ID No: 349, or a fragment or variant thereof.

In an embodiment, the RNA sequence corresponding to the codon optimised DNA sequence of SEQ ID No: 349 is provided herein as SEQ ID No: 350, as follows:

[SEQ ID No: 350]
AUGUGGCAGCCUCCUACACAGAAUGGCACCCAGCUGGAACGGCAUUGGUUCGAGAGCGUUUGGAGAAGCCACGCCGCU
UUCUGCAGCUGCGGAGAUUGCAUCGGACAUCUGCAGCACCUGGCCACCAAUCUGGGUAGACCUCCAGCUCCUCAGCCU
CCUCGAGAUCAGCACCCUCCUCACAUCAGAGGACUGCCUGCACUUCCUGCUCCUCCAAGCAACAGAAACAGCUGGCCU
GGCACAGGCGGAGAUGCUGCUGGCGGAGAAGCUGGUGGAUCUAGAGGUGCCGGCGACGGUGGCGACGGCGAACUUGCU
GAUGAAGAUCUGCUGGACGCUAUCGCCCUGGCCGCUGAA

Hence, preferably the RNA construct comprises a sequence substantially as set out in SEQ ID No: 350, or a fragment or variant thereof.

In one embodiment, the at least one IIP is EBV EBNA1 (P03211; Epstein-Barr nuclear antigen 1 Epstein-Barr virus (strain B95-8)), or an orthologue thereof. One embodiment of the polypeptide sequence of EBV EBNA1 is represented herein as SEQ ID No: 351, as follows:

[SEQ ID No: 351]
MSDEGPGTGPGNGLGEKGDTSGPEGSGGSGPQRRGGDNHGRGRGRGRGRGGGRPGAPGGSGSGPRHRDGVRRPQKRPS
CIGCKGTHGGTGAGAGAGGAGAGGAGAGGGAGAGGGAGGAGGAGGAGAGGGAGAGGGAGGAGGAGAGGGAGAGGGAGG
AGAGGGAGGAGGAGAGGGAGAGGGAGGAGAGGGAGGAGGAGAGGGAGAGGAGGAGGAGAGGAGAGGGAGGAGGAGAGG
AGAGGAGAGGAGAGGAGGAGAGGAGGAGAGGAGGAGAGGGAGGAGAGGGAGGAGAGGAGGAGAGGAGGAGAGGAGGAG
AGGGAGAGGAGAGGGGRGRGGSGGRGRGGSGGRGRGGSGGRRGRGRERARGGSRERARGRGRGRGEKRPRSPSSQSSS
SGSPPRRPPPGRRPFFHPVGEADYFEYHQEGGPDGEPDVPPGAIEQGPADDPGEGPSTGPRGQGDGGRRKKGGWFGKH
RGQGGSNPKFENIAEGLRALLARSHVERTTDEGTWVAGVFVYGGSKTSLYNLRRGTALAIPQCRLTPLSRLPFGMAPG
PGPQPGPLRESIVCYFMVFLQTHIFAEVLKDAIKDLVMTKPAPTCNIRVTVCSFDDGVDLPPWFPPMVEGAAAEGDDG
DDGDEGGDGDEGEEGQE

Therefore, preferably the RNA construct comprises a nucleotide sequence which encodes an amino acid sequence substantially as set out in SEQ ID No: 351, or a variant or fragment thereof.

In one embodiment, the EBV EBNA1 polypeptide is encoded by the DNA nucleotide sequence of SEQ ID No: 352, as follows:

[SEQ ID No: 352]
ATGTCTGACGAGGGGCCAGGTACAGGACCTGGAAATGGCCTAGGAGAGAAGGGAGACACATCTGGACCAGAAGGCTCC
GGCGGCAGTGGACCTCAAAGAAGAGGGGGTGATAACCATGGACGAGGACGGGGAAGAGGACGAGGACGAGGAGGCGGA
AGACCAGGAGCCCCGGGCGGCTCAGGATCAGGGCCAAGACATAGAGATGGTGTCCGGAGACCCCAAAAACGTCCAAGT
TGCATTGGCTGCAAAGGGACCCACGGTGGAACAGGAGCAGGAGCAGGAGCGGGAGGGGCAGGAGCAGGAGGGGCAGGA
GCAGGAGGAGGGGCAGGAGCAGGAGGAGGGGCAGGAGGGGCAGGAGGGGCAGGAGGGGCAGGAGCAGGAGGAGGGGCA
GGAGCAGGAGGAGGGGCAGGAGGGGCAGGAGGGGCAGGAGCAGGAGGAGGGGCAGGAGCAGGAGGAGGGGCAGGAGGG
GCAGGAGCAGGAGGAGGGGCAGGAGGGGCAGGAGGGGCAGGAGCAGGAGGAGGGGCAGGAGCAGGAGGAGGGGCAGGA
GGGGCAGGAGCAGGAGGAGGGGCAGGAGGGGCAGGAGGGGCAGGAGCAGGAGGAGGGGCAGGAGCAGGAGGGGCAGGA
GGGGCAGGAGGGGCAGGAGCAGGAGGGGCAGGAGCAGGAGGAGGGGCAGGAGGGGCAGGAGGGGCAGGAGCAGGAGGG
GCAGGAGCAGGAGGGGCAGGAGCAGGAGGGGCAGGAGCAGGAGGGGCAGGAGGGGCAGGAGCAGGAGGGGCAGGAGGG
GCAGGAGCAGGAGGGGCAGGAGGGGCAGGAGCAGGAGGAGGGGCAGGAGGGGCAGGAGCAGGAGGAGGGGCAGGAGGG
GCAGGAGCAGGAGGGGCAGGAGGGGCAGGAGCAGGAGGGGCAGGAGGGGCAGGAGCAGGAGGGGCAGGAGGGGCAGGA
GCAGGAGGAGGGGCAGGAGCAGGAGGGGCAGGAGCAGGAGGTGGAGGCCGGGGTCGAGGAGGCAGTGGAGGCCGGGGT
CGAGGAGGTAGTGGAGGCCGGGGTCGAGGAGGTAGTGGAGGCCGCCGGGGTAGAGGACGTGAAAGAGCCAGGGGGGGA
AGTCGTGAAAGAGCCAGGGGGAGAGGTCGTGGACGTGGAGAAAAGAGGCCCAGGAGTCCCAGTAGTCAGTCATCATCA
TCCGGGTCTCCACCGCGCAGGCCCCCTCCAGGTAGAAGGCCATTTTTCCACCCTGTAGGGGAAGCCGATTATTTTGAA
TACCACCAAGAAGGTGGCCCAGATGGTGAGCCTGACGTGCCCCCGGGAGCGATAGAGCAGGGCCCCGCAGATGACCCA
GGAGAAGGCCCAAGCACTGGACCCCGGGGTCAGGGTGATGGAGGCAGGCGCAAAAAAGGAGGGTGGTTTGGAAAGCAT
CGTGGTCAAGGAGGTTCCAACCCGAAATTTGAGAACATTGCAGAAGGTTTAAGAGCTCTCCTGGCTAGGAGTCACGTA
GAAAGGACTACCGACGAAGGAACTTGGGTCGCCGGTGTGTTCGTATATGGAGGTAGTAAGACCTCCCTTTACAACCTA
AGGCGAGGAACTGCCCTTGCTATTCCACAATGTCGTCTTACACCATTGAGTCGTCTCCCCTTTGGAATGGCCCCTGGA
CCCGGCCCACAACCTGGCCCGCTAAGGGAGTCCATTGTCTGTTATTTCATGGTCTTTTTACAAACTCATATATTTGCT
GAGGTTTTGAAGGATGCGATTAAGGACCTTGTTATGACAAAGCCCGCTCCTACCTGCAATATCAGGGTGACTGTGTGC
AGCTTTGACGATGGAGTAGATTTGCCTCCCTGGTTTCCACCTATGGTGGAAGGGGCTGCCGCGGAGGGTGATGACGGA
GATGACGGAGATGAAGGAGGTGATGGAGATGAGGGTGAGGAAGGGCAGGAGTGA

Accordingly, preferably the EBV EBNA1 polypeptide is encoded by the DNA nucleotide sequence substantially as set out in SEQ ID NO: 352, or a variant or fragment thereof.

One embodiment of codon optimised nucleic acid (DNA) encoding the EBV EBNA1 polypeptide is provided herein as SEQ ID No: 353, as follows:

[SEQ ID No: 353]
ATGTCCGATGAAGGCCCTGGAACAGGCCCTGGCAATGGACTGGGAGAGAAGGGCGATACAAGCGGCCCTGAAGGTTCT
GGCGGATCTGGCCCTCAAAGAAGAGGCGGCGATAATCACGGCAGAGGACGCGGAAGAGGTAGAGGCAGAGGCGGAGGT
AGACCTGGTGCTCCTGGTGGTTCTGGCTCTGGCCCTAGACATAGAGATGGCGTCAGACGGCCTCAGAAGAGGCCTTCT
TGTATCGGCTGCAAGGGCACACATGGCGGAACAGGTGCTGGTGCTGGCGCAGGCGGAGCAGGCGCTGGTGGTGCAGGC
GCTGGCGGCGGTGCCGGTGCAGGCGGCGGAGCTGGTGGCGCTGGCGGTGCTGGCGGAGCTGGTGCAGGCGGAGGTGCC
GGCGCTGGTGGCGGAGCAGGCGGAGCTGGCGGAGCCGGCGCTGGCGGTGGCGCTGGTGCCGGCGGAGGCGCAGGCGGC
GCTGGTGCTGGTGGTGGTGCTGGCGGCGCAGGCGGTGCAGGCGCAGGCGGAGGCGCTGGCGCTGGCGGTGGTGCAGGC
GGTGCTGGCGCTGGCGGCGGTGCTGGCGGAGCCGGTGGTGCTGGTGCTGGTGGCGGAGCTGGCGCTGGCGGAGCTGGC
GGTGCAGGCGGCGCAGGCGCTGGTGGCGCTGGCGCAGGCGGTGGCGCTGGCGGAGCAGGCGGAGCTGGCGCTGGCGGC
GCAGGCGCAGGCGGAGCCGGTGCTGGCGGAGCTGGTGCTGGTGGTGCAGGCGGAGCTGGTGCCGGTGGCGCTGGTGGT
GCCGGTGCCGGTGGTGCCGGCGGAGCCGGCGCAGGCGGCGGTGCAGGCGGAGCAGGCGCAGGCGGCGGAGCTGGTGGT
GCCGGCGCAGGCGGCGCTGGTGGTGCTGGTGCCGGCGGAGCTGGTGGCGCAGGCGCTGGCGGTGCAGGCGGTGCCGGT
GCCGGTGGTGGTGCAGGCGCAGGCGGTGCTGGTGCCGGCGGTGGCGGAAGAGGAAGAGGTGGTAGCGGAGGCCGAGGA
CGAGGCGGAAGTGGTGGTCGTGGTAGAGGCGGCAGCGGAGGAAGAAGAGGACGGGGTAGAGAACGAGCTAGAGGCGGA
TCTAGAGAGAGAGCCCGAGGCAGAGGAAGAGGCCGCGGAGAGAAAAGACCTAGAAGCCCTAGCAGCCAGAGCAGCTCT
AGCGGATCTCCACCTAGAAGGCCACCTCCAGGCAGACGGCCATTCTTTCACCCTGTGGGCGAAGCCGACTACTTCGAG
TACCACCAAGAAGGCGGACCTGACGGCGAACCTGATGTTCCTCCTGGCGCCATTGAACAGGGCCCAGCTGATGATCCT
GGCGAGGGACCTTCTACAGGCCCTAGAGGACAAGGCGACGGCGGCAGACGAAAGAAAGGCGGATGGTTCGGCAAGCAC
AGAGGCCAAGGTGGCAGCAACCCCAAGTTCGAGAATATCGCCGAGGGCCTGAGAGCCCTGCTGGCCAGATCTCACGTG
GAAAGAACCACCGACGAAGGCACATGGGTGGCAGGCGTGTTCGTTTACGGCGGCTCTAAGACCAGCCTGTACAACCTG
AGAAGAGGCACAGCCCTGGCCATTCCTCAGTGCAGACTGACCCCTCTGAGCAGACTGCCTTTTGGCATGGCTCCTGGA
CCTGGACCTCAACCTGGACCACTGAGAGAATCCATCGTGTGCTACTTCATGGTGTTTCTGCAGACCCACATCTTCGCC
GAGGTGCTGAAGGACGCCATCAAGGACCTGGTCATGACAAAGCCCGCTCCTACCTGCAACATCAGAGTGACCGTGTGC
AGCTTCGACGACGGCGTTGACCTGCCTCCTTGGTTTCCTCCAATGGTGGAAGGCGCTGCTGCCGAAGGCGACGATGGC
GACGACGGCGACGAAGGTGGCGACGGCGACGAGGGCGAAGAAGGACAAGAGTAA

Hence, preferably the RNA construct is encoded by a DNA sequence substantially as set out in SEQ ID No: 353, or a fragment or variant thereof.

In an embodiment, the RNA sequence corresponding to the codon optimised DNA sequence of SEQ ID No: 353 is provided herein as SEQ ID No: 354, as follows:

[SEQ ID No: 354]
AUGUCCGAUGAAGGCCCUGGAACAGGCCCUGGCAAUGGACUGGGAGAGAAGGGCGAUACAAGCGGCCCUGAAGGUUCU
GGCGGAUCUGGCCCUCAAAGAAGAGGCGGCGAUAAUCACGGCAGAGGACGCGGAAGAGGUAGAGGCAGAGGCGGAGGU
AGACCUGGUGCUCCUGGUGGUUCUGGCUCUGGCCCUAGACAUAGAGAUGGCGUCAGACGGCCUCAGAAGAGGCCUUCU
UGUAUCGGCUGCAAGGGCACACAUGGCGGAACAGGUGCUGGUGCUGGCGCAGGCGGAGCAGGCGCUGGUGGUGCAGGC
GCUGGCGGCGGUGCCGGUGCAGGCGGCGGAGCUGGUGGCGCUGGCGGUGCUGGCGGAGCUGGUGCAGGCGGAGGUGCC
GGCGCUGGUGGCGGAGCAGGCGGAGCUGGCGGAGCCGGCGCUGGCGGUGGCGCUGGUGCCGGCGGAGGCGCAGGCGGC
GCUGGUGCUGGUGGUGGUGCUGGCGGCGCAGGCGGUGCAGGCGCAGGCGGAGGCGCUGGCGCUGGCGGUGGUGCAGGC
GGUGCUGGCGCUGGCGGCGGUGCUGGCGGAGCCGGUGGUGCUGGUGCUGGUGGCGGAGCUGGCGCUGGCGGAGCUGGC
GGUGCAGGCGGCGCAGGCGCUGGUGGCGCUGGCGCAGGCGGUGGCGCUGGCGGAGCAGGCGGAGCUGGCGCUGGCGGC
GCAGGCGCAGGCGGAGCCGGUGCUGGCGGAGCUGGUGCUGGUGGUGCAGGCGGAGCUGGUGCCGGUGGCGCUGGUGGU
GCCGGUGCCGGUGGUGCCGGCGGAGCCGGCGCAGGCGGCGGUGCAGGCGGAGCAGGCGCAGGCGGCGGAGCUGGUGGU
GCCGGCGCAGGCGGCGCUGGUGGUGCUGGUGCCGGCGGAGCUGGUGGCGCAGGCGCUGGCGGUGCAGGCGGUGCCGGU
GCCGGUGGUGGUGCAGGCGCAGGCGGUGCUGGUGCCGGCGGUGGCGGAAGAGGAAGAGGUGGUAGCGGAGGCCGAGGA
CGAGGCGGAAGUGGUGGUCGUGGUAGAGGCGGCAGCGGAGGAAGAAGAGGACGGGGUAGAGAACGAGCUAGAGGCGGA
UCUAGAGAGAGAGCCCGAGGCAGAGGAAGAGGCCGCGGAGAGAAAAGACCUAGAAGCCCUAGCAGCCAGAGCAGCUCU
AGCGGAUCUCCACCUAGAAGGCCACCUCCAGGCAGACGGCCAUUCUUUCACCCUGUGGGCGAAGCCGACUACUUCGAG
UACCACCAAGAAGGCGGACCUGACGGCGAACCUGAUGUUCCUCCUGGCGCCAUUGAACAGGGCCCAGCUGAUGAUCCU
GGCGAGGGACCUUCUACAGGCCCUAGAGGACAAGGCGACGGCGGCAGACGAAAGAAAGGCGGAUGGUUCGGCAAGCAC
AGAGGCCAAGGUGGCAGCAACCCCAAGUUCGAGAAUAUCGCCGAGGGCCUGAGAGCCCUGCUGGCCAGAUCUCACGUG
GAAAGAACCACCGACGAAGGCACAUGGGUGGCAGGCGUGUUCGUUUACGGCGGCUCUAAGACCAGCCUGUACAACCUG
AGAAGAGGCACAGCCCUGGCCAUUCCUCAGUGCAGACUGACCCCUCUGAGCAGACUGCCUUUUGGCAUGGCUCCUGGA
CCUGGACCUCAACCUGGACCACUGAGAGAAUCCAUCGUGUGCUACUUCAUGGUGUUUCUGCAGACCCACAUCUUCGCC
GAGGUGCUGAAGGACGCCAUCAAGGACCUGGUCAUGACAAAGCCCGCUCCUACCUGCAACAUCAGAGUGACCGUGUGC
AGCUUCGACGACGGCGUUGACCUGCCUCCUUGGUUUCCUCCAAUGGUGGAAGGCGCUGCUGCCGAAGGCGACGAUGGC
GACGACGGCGACGAAGGUGGCGACGGCGACGAGGGCGAAGAAGGACAAGAGUAA

Hence, preferably the RNA construct comprises a sequence substantially as set out in SEQ 50 ID No: 354, or a fragment or variant thereof.

In one embodiment, the at least one IIP is BDV P protein (POC799; Phosphoprotein Borna disease virus (strain V)), or an orthologue thereof. It is believed that this IIP acts as a decoy protein for phosphorylation by TBK, thus effecting reduction in TBK activity and activation of IRF3 and 7. One embodiment of the polypeptide sequence of BDV P protein is represented herein as SEQ ID No: 355, as follows:

[SEQ ID No: 355]
MATRPSSLVDSLEDEEDPQTLRRERPGSPRPRKVPRNALTQPVDQLLKDLRKNPSMISDPDQRTGREQLSNDELIKKL
VTELAENSMIEAEEVRGTLGDISARIEAGFESLSALQVETIQTAQRCDHSDSIRILGENIKILDRSMKTMMETMKLMM
EKVDLLYASTAVGTSAPMLPSHPAPPRIYPQLPSAPTTDEWDIIP

Therefore, preferably the RNA construct comprises a nucleotide sequence which encodes an amino acid sequence substantially as set out in SEQ ID No: 355, or a variant or fragment thereof.

In one embodiment, the BDV P polypeptide is encoded by the DNA nucleotide sequence of SEQ ID No: 356, as follows:

[SEQ ID No: 356]
ATGGCAACGCGACCATCGAGTCTGGTCGACTCCCTGGAGGACGAAGAAGATCCCCAGACACTACGACGGGAACGACCG
GGGTCACCAAGACCACGGAAGGTCCCAAGGAATGCATTGACCCAACCAGTAGACCAGCTCCTGAAGGACCTCAGGAAG
AACCCCTCCATGATCTCAGACCCAGACCAGCGAACCGGAAGGGAGCAGCTGTCGAATGATGAGCTAATCAAGAAGTTA
GTGACGGAGCTGGCCGAGAATAGCATGATCGAGGCTGAGGAGGTGCGGGGCACTCTTGGAGACATCTCGGCTCGTATC
GAGGCAGGGTTTGAGTCCCTGTCCGCCCTCCAAGTGGAAACCATCCAGACAGCTCAGCGGTGCGATCACTCCGACAGC
ATCAGGATCCTCGGCGAGAACATCAAGATACTAGATCGCTCCATGAAGACAATGATGGAGACAATGAAGCTCATGATG
GAGAAGGTGGATCTCCTCTACGCATCAACCGCCGTTGGGACCTCTGCACCCATGTTGCCCTCCCATCCTGCACCTCCG
CGCATTTATCCCCAGCTCCCAAGTGCCCCGACAACGGATGAATGGGACATCATACCA

Accordingly, preferably the BDV P polypeptide is encoded by the DNA nucleotide sequence substantially as set out in SEQ ID NO: 356, or a variant or fragment thereof.

One embodiment of codon optimised nucleic acid (DNA) encoding the BDV P polypeptide is provided herein as SEQ ID No: 357, as follows:

[SEQ ID No: 357]
ATGGCCACAAGACCTAGCAGCCTGGTGGACAGCCTGGAAGATGAGGAAGATCCCCAGACACTGCGGAGAGAGAGGCCT
GGATCTCCCAGACCTAGAAAGGTGCCCAGAAACGCCCTGACACAGCCCGTTGATCAGCTGCTGAAGGACCTGAGAAAG
AACCCCAGCATGATCAGCGACCCCGACCAGAGAACCGGAAGAGAGCAGCTGTCTAACGACGAGCTGATTAAGAAGCTG
GTCACCGAGCTGGCCGAGAACTCCATGATTGAGGCCGAAGAAGTGCGGGGCACCCTGGGCGATATCTCTGCCAGAATC
GAGGCCGGCTTTGAGTCTCTGAGCGCCCTGCAGGTTGAGACAATCCAGACAGCCCAGAGATGCGACCACAGCGACAGC
ATCAGAATCCTGGGCGAGAACATCAAGATCCTGGATCGGAGCATGAAGACCATGATGGAAACCATGAAGCTGATGATG
GAAAAGGTGGACCTGCTGTACGCCAGCACAGCCGTGGGAACATCTGCTCCCATGCTGCCTTCTCACCCCGCTCCTCCA
AGAATCTACCCTCAGCTGCCTAGCGCTCCCACCACCGATGAGTGGGATATCATCCCT

Hence, preferably the RNA construct is encoded by a DNA sequence substantially as set out in SEQ ID No: 357, or a fragment or variant thereof.

In an embodiment, the RNA sequence corresponding to the codon optimised DNA sequence of SEQ ID No: 357 is provided herein as SEQ ID No: 358, as follows:

[SEQ ID No: 358]
AUGGCCACAAGACCUAGCAGCCUGGUGGACAGCCUGGAAGAUGAGGAAGAUCCCCAGACACUGCGGAGAGAGAGGCCU
GGAUCUCCCAGACCUAGAAAGGUGCCCAGAAACGCCCUGACACAGCCCGUUGAUCAGCUGCUGAAGGACCUGAGAAAG
AACCCCAGCAUGAUCAGCGACCCCGACCAGAGAACCGGAAGAGAGCAGCUGUCUAACGACGAGCUGAUUAAGAAGCUG
GUCACCGAGCUGGCCGAGAACUCCAUGAUUGAGGCCGAAGAAGUGCGGGGCACCCUGGGCGAUAUCUCUGCCAGAAUC
GAGGCCGGCUUUGAGUCUCUGAGCGCCCUGCAGGUUGAGACAAUCCAGACAGCCCAGAGAUGCGACCACAGCGACAGC
AUCAGAAUCCUGGGCGAGAACAUCAAGAUCCUGGAUCGGAGCAUGAAGACCAUGAUGGAAACCAUGAAGCUGAUGAUG
GAAAAGGUGGACCUGCUGUACGCCAGCACAGCCGUGGGAACAUCUGCUCCCAUGCUGCCUUCUCACCCCGCUCCUCCA
AGAAUCUACCCUCAGCUGCCUAGCGCUCCCACCACCGAUGAGUGGGAUAUCAUCCCU

Hence, preferably the RNA construct comprises a sequence substantially as set out in SEQ ID No: 358, or a fragment or variant thereof.

In one embodiment, the at least one IIP is HPV E7 (P03129; Protein E7 Human papillomavirus type 16), or an orthologue thereof. One embodiment of the polypeptide sequence of HPV E7 is represented herein as SEQ ID No: 359, as follows:

[SEQ ID No: 359]
MHGDTPTLHEYMLDLQPETTDLYCYEQLNDSSEEEDEIDGPAGQAEPDRAHYNIVTFCCKCDSTLRLCVQSTHVDIRT
LEDLLMGTLGIVCPICSQKP

Therefore, preferably the RNA construct comprises a nucleotide sequence which encodes an amino acid sequence substantially as set out in SEQ ID No: 359, or a variant or fragment thereof.

In one embodiment, the HPV E7 polypeptide is encoded by the DNA nucleotide sequence of SEQ ID No: 360, as follows:

[SEQ ID No: 360]
ATGCATGGAGATACACCTACATTGCATGAATATATGTTAGATTTGCAACCAGAGACAACTGATCTCTACTGTTATGAG
CAATTAAATGACAGCTCAGAGGAGGAGGATGAAATAGATGGTCCAGCTGGACAAGCAGAACCGGACAGAGCCCATTAC
AATATTGTAACCTTTTGTTGCAAGTGTGACTCTACGCTTCGGTTGTGCGTACAAAGCACACACGTAGACATTCGTACT
TTGGAAGACCTGTTAATGGGCACACTAGGAATTGTGTGCCCCATCTGTTCTCAGAAACCA

Accordingly, preferably the HPV E7 polypeptide is encoded by the DNA nucleotide sequence substantially as set out in SEQ ID NO: 360, or a variant or fragment thereof.

One embodiment of codon optimised nucleic acid (DNA) encoding the HPV E7 polypeptide is provided herein as SEQ ID No: 361, as follows:

[SEQ ID No: 361]
ATGCACGGCGATACCCCTACACTGCACGAGTACATGCTGGACCTGCAGCCTGAGACAACCGACCTGTACTGCTACGAG
CAGCTGAACGACAGCAGCGAGGAAGAGGACGAGATTGACGGACCTGCCGGACAGGCCGAACCTGATAGAGCCCACTAC
AATATCGTGACCTTCTGCTGCAAGTGCGACAGCACCCTGAGACTGTGTGTGCAGAGCACCCACGTGGACATCAGAACC
CTGGAAGATCTGCTGATGGGCACCCTGGGCATCGTGTGCCCTATCTGTTCTCAGAAGCCC

Hence, preferably the RNA construct is encoded by a DNA sequence substantially as set out in SEQ ID No: 361, or a fragment or variant thereof.

In an embodiment, the RNA sequence corresponding to the codon optimised DNA sequence of SEQ ID No: 361 is provided herein as SEQ ID No: 362, as follows:

[SEQ ID No: 362]
AUGCACGGCGAUACCCCUACACUGCACGAGUACAUGCUGGACCUGCAGCCUGAGACAACCGACCUGUACUGCUACGAG
CAGCUGAACGACAGCAGCGAGGAAGAGGACGAGAUUGACGGACCUGCCGGACAGGCCGAACCUGAUAGAGCCCACUAC
AAUAUCGUGACCUUCUGCUGCAAGUGCGACAGCACCCUGAGACUGUGUGUGCAGAGCACCCACGUGGACAUCAGAACC
CUGGAAGAUCUGCUGAUGGGCACCCUGGGCAUCGUGUGCCCUAUCUGUUCUCAGAAGCCC

Hence, preferably the RNA construct comprises a sequence substantially as set out in SEQ ID No: 362, or a fragment or variant thereof.

In one embodiment, the at least one IIP is Arenavirus NP1 protein (A0A2H4RDN2; Nucleoprotein Arenavirus sp.), or an orthologue thereof. One embodiment of the polypeptide sequence of Arenavirus NP1 protein is represented herein as SEQ ID No: 363, as follows:

[SEQ ID No: 363]
MSNSKEVKSFLWTQALRRELSPYCTSVKLQVIKDAQSLLHSLDFSEVSNVQRLMRKDKRDDGDLKRLRDLNQAVNNLV
ELKSQQQKNVLSVGQLSSDDLLVLAADIDRLKAKITRTERPQSNGVYMGNLTAQQLEQRKKLLELVGMTRPNLRAGSD
GVVRVWDVKNPDLLNNQFGTMPSLTIACMTKQGQSDINDVVQALTDLGLIYTAKYPNSSDLDQLVKTHPVLGIIDTEK
SAINVSGYNFSLSAAVKAGACMLDGGNMLETIKVTPQNIDPILKKTLAVKKSVGMFVSDTPGDRNPYENLLYKICLSG
NGWPYIASRTSILGRAWDNTVVDLGSSNPITKPLNQQARDKVPGLQQTVGLTYSQIMCLKDIMTGMDPTSKTWIDIEG
RAEDPVEIAIYQPAGGQYIHFYREPTDAKQFKQDSKYSHGIDIVDLFRVQPGLTSAVIESLPKGMVLICQGSEDIRKL
LDSQGRRDIKLIDVMMSKIDARKFENEVWDDLKTLCNMHTGVVHEKKKRGGKQEITPHCALLDCIMYEAATQGSYKTP
KLTPLLPTDLVFRAGAPKVTL

Therefore, preferably the RNA construct comprises a nucleotide sequence which encodes an amino acid sequence substantially as set out in SEQ ID No: 363, or a variant or fragment thereof.

In one embodiment, the Arenavirus NP1 polypeptide is encoded by the DNA nucleotide sequence of SEQ ID No: 364, as follows:

[SEQ ID No: 364]
ATGAGCAACTCCAAGGAGGTGAAGTCCTTTCTTTGGACTCAAGCCCTTAGGAGGGAATTATCACCATACTGCACAAGT
GTCAAACTTCAAGTTATCAAGGATGCTCAGAGTCTCCTTCACAGCTTGGATTTCTCAGAAGTGAGTAATGTACAACGC
TTGATGAGGAAGGACAAAAGGGATGACGGTGACTTGAAGAGGCTGAGAGATTTAAATCAGGCAGTCAACAATCTTGTT
GAACTAAAATCTCAACAGCAGAAAAATGTCTTAAGTGTGGGGCAGCTGTCATCTGATGACCTTTTAGTCCTCGCTGCT
GACATTGACAGGCTGAAAGCAAAGATAACCAGGACAGAGAGGCCCCAATCTAATGGGGTCTACATGGGGAACCTCACA
GCTCAGCAACTTGAACAACGAAAGAAGCTCCTAGAGTTGGTGGGGATGACCAGACCAAACTTAAGAGCTGGTTCTGAT
GGTGTTGTCAGGGTGTGGGACGTGAAGAATCCTGATCTCTTGAATAACCAATTTGGCACAATGCCCAGTCTAACGATT
GCCTGCATGACAAAACAAGGACAATCAGACATAAATGATGTTGTTCAGGCATTAACTGACTTGGGGCTAATTTATACA
GCTAAGTACCCAAATTCATCAGATCTTGATCAACTTGTCAAAACCCATCCAGTTTTGGGCATCATAGACACAGAAAAA
TCTGCCATCAATGTTTCAGGTTACAACTTCAGCCTGTCAGCTGCAGTTAAGGCAGGTGCATGTATGCTAGATGGGGGT
AACATGCTCGAGACCATAAAGGTAACACCTCAGAATATTGATCCAATTCTGAAGAAGACTCTGGCAGTTAAAAAGTCT
GTTGGCATGTTTGTCTCAGACACACCAGGTGACAGAAACCCATATGAAAACTTACTATACAAGATCTGCCTCTCAGGC
AGTAACCCAATCACAAAGCCCCTCAATCAGCAAGCTAGAGACAAAGTTCCTGGTTTGCAGCAAACAGTTGGACTCACA
TACTCACAAATCATGTGTCTCAAAGACATAATGACCGGTATGGACCCGACAAGTAAGACTTGGATTGACATTGAGGGC
AGGGCTGAGGACCCAGTGGAGATTGCCATCTACCAGCCAGCTGGTGGGCAATATATTCATTTCTACAGAGAACCAACA
GATGCCAAGCAATTTAAGCAGGATTCTAAGTACTCACATGGCATTGACATTGTTGACCTGTTTAGGGTGCAACCAGGC
CTTACAAGTGCTGTGATAGAGAGTCTACCGAAAGGGATGGTCTTAACTTGTCAGGGATCTGAGGACATAAGAAAGCTG
TTAGATAGTCAGGGGCGCCGAGACATCAAGTTAATTGATGTGATGATGAGCAAGATTGATGCACGGAAGTTTGAAAAT
GAGGTCTGGGATGATCTTAAAACACTGTGCAACATGCACACTGGGGTGGTCCATGAGAAGAAGAAGAGAGGTGGTAAA
CAAGAAATAACACCTCACTGTGCACTTCTAGACTGCATTATGTATGAGGCAGCCACCCAGGGGTCATACAAGACCCCC
AAATTAACACCTCTGCTACCAACTGACTTGGTGTTTAGAGCAGGAGCACCCAAAGTCACTCTG

Accordingly, preferably the Arenavirus NP1 polypeptide is encoded by the DNA nucleotide sequence substantially as set out in SEQ ID NO: 364, or a variant or fragment thereof.

One embodiment of codon optimised nucleic acid (DNA) encoding the Arenavirus NP1 polypeptide is provided herein as SEQ ID No: 365, as follows:

[SEQ ID No: 365]
ATGAGCAACAGCAAAGAAGTCAAGAGCTTCCTCTGGACACAGGCCCTGAGAAGAGAGCTGAGCCCTTACTGCACCAGC
GTGAAGCTGCAAGTGATCAAGGACGCCCAGAGCCTGCTGCACAGCCTGGATTTTTCCGAGGTGTCCAACGTGCAGCGG
CTGATGCGGAAGGACAAGAGAGATGACGGCGACCTGAAGCGGCTGAGGGATCTGAATCAGGCCGTGAACAACCTGGTG
GAACTGAAGTCCCAGCAGCAGAAAAACGTGCTGAGCGTGGGCCAGCTGAGCAGCGACGATCTGCTTGTTCTGGCCGCC
GACATCGACAGACTGAAGGCCAAGATCACCAGAACCGAGCGGCCTCAGAGCAACGGCGTGTACATGGGAAATCTGACA
GCCCAGCAGCTGGAACAGCGGAAGAAACTGCTGGAACTCGTGGGCATGACCCGGCCTAATCTGAGAGCTGGCTCTGAT
GGCGTCGTCAGAGTGTGGGACGTGAAGAACCCCGACCTGCTGAACAACCAGTTCGGCACCATGCCTAGCCTGACAATC
GCCTGCATGACCAAGCAGGGCCAGAGCGACATCAACGATGTGGTGCAGGCACTGACCGACCTGGGCCTGATCTACACC
GCCAAGTATCCCAACAGCAGCGACCTGGATCAGCTGGTCAAGACACACCCTGTGCTGGGCATCATCGACACCGAGAAG
TCCGCCATCAACGTGTCCGGCTACAACTTCTCTCTGTCTGCCGCCGTGAAAGCCGGCGCTTGTATGCTGGATGGCGGC
AACATGCTGGAAACCATCAAAGTGACCCCTCAGAACATCGACCCCATCCTGAAGAAAACCCTGGCCGTGAAGAAAAGC
GTGGGGATGTTCGTGTCTGACACCCCTGGCGACAGAAACCCCTACGAGAACCTGCTGTACAAGATCTGCCTGAGCGGC
AACGGCTGGCCCTATATCGCCAGCAGAACCAGCATTCTGGGCAGAGCCTGGGACAACACCGTGGTGGATCTGGGCAGC
AGCAACCCCATCACCAAGCCTCTGAACCAGCAGGCCAGAGATAAGGTGCCAGGCCTGCAGCAGACAGTGGGCCTGACA
TACAGCCAGATCATGTGCCTGAAGGACATCATGACCGGCATGGACCCCACCAGCAAGACATGGATCGACATCGAGGGC
AGAGCTGAGGACCCTGTGGAAATCGCCATCTACCAACCTGCCGGCGGACAGTACATCCACTTCTACAGAGAGCCCACC
GACGCCAAGCAGTTCAAGCAGGACAGCAAGTACAGCCACGGCATCGATATCGTGGACCTGTTCAGAGTGCAGCCCGGA
CTGACATCTGCCGTGATCGAGTCTCTGCCCAAAGGCATGGTCCTGACCTGTCAGGGCAGCGAGGACATCAGAAAGCTG
CTCGACAGCCAGGGCAGAAGAGACATCAAGCTGATCGACGTGATGATGAGCAAGATCGACGCCCGGAAGTTCGAGAAC
GAAGTGTGGGATGACCTGAAAACCCTCTGCAACATGCACACCGGCGTGGTGCACGAGAAGAAGAAGAGAGGCGGCAAG
CAAGAGATCACCCCTCACTGTGCTCTGCTGGACTGCATTATGTACGAGGCCGCCACACAGGGCAGCTACAAGACCCCT
AAACTGACCCCTCTGCTGCCTACCGATCTGGTGTTTAGAGCCGGCGCACCCAAAGTGACACTG

Hence, preferably the RNA construct is encoded by a DNA sequence substantially as set out in SEQ ID No: 365, or a fragment or variant thereof.

In an embodiment, the RNA sequence corresponding to the codon optimised DNA sequence of SEQ ID No: 365 is provided herein as SEQ ID No: 366, as follows:

[SEQ ID No: 366]
AUGAGCAACAGCAAAGAAGUCAAGAGCUUCCUCUGGACACAGGCCCUGAGAAGAGAGCUGAGCCCUUACUGCACCAGC
GUGAAGCUGCAAGUGAUCAAGGACGCCCAGAGCCUGCUGCACAGCCUGGAUUUUUCCGAGGUGUCCAACGUGCAGCGG
CUGAUGCGGAAGGACAAGAGAGAUGACGGCGACCUGAAGCGGCUGAGGGAUCUGAAUCAGGCCGUGAACAACCUGGUG
GAACUGAAGUCCCAGCAGCAGAAAAACGUGCUGAGCGUGGGCCAGCUGAGCAGCGACGAUCUGCUUGUUCUGGCCGCC
GACAUCGACAGACUGAAGGCCAAGAUCACCAGAACCGAGCGGCCUCAGAGCAACGGCGUGUACAUGGGAAAUCUGACA
GCCCAGCAGCUGGAACAGCGGAAGAAACUGCUGGAACUCGUGGGCAUGACCCGGCCUAAUCUGAGAGCUGGCUCUGAU
GGCGUCGUCAGAGUGUGGGACGUGAAGAACCCCGACCUGCUGAACAACCAGUUCGGCACCAUGCCUAGCCUGACAAUC
GCCUGCAUGACCAAGCAGGGCCAGAGCGACAUCAACGAUGUGGUGCAGGCACUGACCGACCUGGGCCUGAUCUACACC
GCCAAGUAUCCCAACAGCAGCGACCUGGAUCAGCUGGUCAAGACACACCCUGUGCUGGGCAUCAUCGACACCGAGAAG
UCCGCCAUCAACGUGUCCGGCUACAACUUCUCUCUGUCUGCCGCCGUGAAAGCCGGCGCUUGUAUGCUGGAUGGCGGC
AACAUGCUGGAAACCAUCAAAGUGACCCCUCAGAACAUCGACCCCAUCCUGAAGAAAACCCUGGCCGUGAAGAAAAGC
GUGGGGAUGUUCGUGUCUGACACCCCUGGCGACAGAAACCCCUACGAGAACCUGCUGUACAAGAUCUGCCUGAGCGGC
AACGGCUGGCCCUAUAUCGCCAGCAGAACCAGCAUUCUGGGCAGAGCCUGGGACAACACCGUGGUGGAUCUGGGCAGC
AGCAACCCCAUCACCAAGCCUCUGAACCAGCAGGCCAGAGAUAAGGUGCCAGGCCUGCAGCAGACAGUGGGCCUGACA
UACAGCCAGAUCAUGUGCCUGAAGGACAUCAUGACCGGCAUGGACCCCACCAGCAAGACAUGGAUCGACAUCGAGGGC
AGAGCUGAGGACCCUGUGGAAAUCGCCAUCUACCAACCUGCCGGCGGACAGUACAUCCACUUCUACAGAGAGCCCACC
GACGCCAAGCAGUUCAAGCAGGACAGCAAGUACAGCCACGGCAUCGAUAUCGUGGACCUGUUCAGAGUGCAGCCCGGA
CUGACAUCUGCCGUGAUCGAGUCUCUGCCCAAAGGCAUGGUCCUGACCUGUCAGGGCAGCGAGGACAUCAGAAAGCUG
CUCGACAGCCAGGGCAGAAGAGACAUCAAGCUGAUCGACGUGAUGAUGAGCAAGAUCGACGCCCGGAAGUUCGAGAAC
GAAGUGUGGGAUGACCUGAAAACCCUCUGCAACAUGCACACCGGCGUGGUGCACGAGAAGAAGAAGAGAGGCGGCAAG
CAAGAGAUCACCCCUCACUGUGCUCUGCUGGACUGCAUUAUGUACGAGGCCGCCACACAGGGCAGCUACAAGACCCCU
AAACUGACCCCUCUGCUGCCUACCGAUCUGGUGUUUAGAGCCGGCGCACCCAAAGUGACACUG

Hence, preferably the RNA construct comprises a sequence substantially as set out in SEQ ID No: 366, or a fragment or variant thereof.

In one embodiment, the at least one IIP is HCV NS3 protein (P27958; Genome polyprotein Hepatitis C virus genotype 1a (isolate H77)), or an orthologue thereof. One embodiment of the polypeptide sequence of HCV NS3 protein is represented herein as SEQ ID No: 367, as follows:

[SEQ ID No: 367]
APITAYAQQTRGLLGCIITSLTGRDKNQVEGEVQIVSTATQTFLATCINGVCWTVYHGAGTRTIASPKGPVIQTYTNV
DQDLVGWPAPQGSRSLTPCTCGSSDLYLVTRHADVIPVRRRGDSRGSLLSPRPISYLKGSSGGPLLCPTGHAVGLFRA
AVCTRGVAKAVDFIPVENLETTMRSPVFTDNSSPPAVPQSFQVAHLHAPTGSGKSTKVPAAYAAKGYKVLVLNPSVAA
TLGFGAYMSKAHGVDPNIRTGVRTITTGSPITYSTYGKFLADAGCSGGAYDIIICDECHSTDATSISGIGTVLDQAET
AGARLVVLATATPPGSVTVSHPNIEEVALSTTGEIPFYGKAIPLEVIKGGRHLIFCHSKKKCDELAAKLVALGINAVA
YYRGLDVSVIPTSGDVVVVSTDALMTGFTGDFDSVIDCNTCVTQTVDFSLDPTFTIETTTLPQDAVSRTQRRGRTGRG
KPGIYRFVAPGERPSGMFDSSVLCECYDAGCAWYELTPAETTVRLRAYMNTPGLPVCQDHLGFWEGVFTGLTHIDAHF
LSQTKQSGENFPYLVAYQATVCARAQAPPPSWDQMRKCLIRLKPTLHGPTPLLYRLGAVQNEVTLTHPITKYIMTCMS
ADLEVVT

Therefore, preferably the RNA construct comprises a nucleotide sequence which encodes an amino acid sequence substantially as set out in SEQ ID No: 367, or a variant or fragment thereof.

In one embodiment, the HCV NS3 polypeptide is encoded by the DNA nucleotide sequence of SEQ ID No: 368, as follows:

[SEQ ID No: 368]
GCGCCCATCACGGCGTACGCCCAGCAGACGAGAGGCCTCCTAGGGTGTATAATCACCAGCCTGACTGGCCGGGACAAA
AACCAAGTGGAGGGTGAGGTCCAGATCGTGTCAACTGCTACCCAGACCTTCCTGGCAACGTGCATCAATGGGGTATGC
TGGACTGTCTACCACGGGGCCGGAACGAGGACCATCGCATCACCCAAGGGTCCTGTCATCCAGACGTATACCAATGTG
GATCAAGACCTCGTGGGCTGGCCCGCTCCTCAAGGTTCCCGCTCATTGACACCCTGCACCTGCGGCTCCTCGGACCTT
TACCTGGTCACGAGGCACGCCGATGTCATTCCCGTGCGCCGGCGAGGTGATAGCAGGGGTAGCCTGCTTTCGCCCCGG
CCCATTTCCTACTTGAAAGGCTCCTCGGGGGGTCCGCTGTTGTGCCCCACGGGACACGCCGTGGGCCTATTCAGGGCC
GCGGTGTGCACCCGTGGAGTGGCTAAGGCGGTGGACTTTATCCCTGTGGAGAACCTAGAGACAACCATGAGATCCCCG
GTGTTCACGGACAACTCCTCTCCACCAGCAGTGCCCCAGAGCTTCCAGGTGGCCCACCTGCATGCTCCCACCGGCAGC
GGTAAGAGCACCAAGGTCCCGGCTGCGTACGCAGCCAAGGGCTACAAGGTGTTGGTGCTCAACCCCTCTGTTGCTGCA
ACACTGGGCTTTGGTGCTTACATGTCCAAGGCCCATGGGGTTGATCCTAATATCAGGACCGGGGTGAGAACAATTACC
ACTGGCAGCCCCATCACGTACTCCACCTACGGCAAGTTCCTTGCCGACGCCGGGTGCTCAGGAGGTGCTTATGACATA
ATAATTTGTGACGAGTGCCACTCCACGGATGCCACATCCATCTCGGGCATCGGCACTGTCCTTGACCAAGCAGAGACT
GCGGGGGCGAGACTGGTTGTGCTCGCCACTGCTACCCCTCCGGGCTCCGTCACTGTGTCCCATCCTAACATCGAGGAG
GTTGCTCTGTCCACCACCGGAGAGATCCCCTTTTACGGCAAGGCTATCCCCCTCGAGGTGATCAAGGGGGGAAGACAT
CTCATCTTCTGCCACTCAAAGAAGAAGTGCGACGAGCTCGCCGCGAAGCTGGTCGCATTGGGCATCAATGCCGTGGCC
TACTACCGCGGTCTTGACGTGTCTGTCATCCCGACCAGCGGCGATGTTGTCGTCGTGTCGACCGATGCTCTCATGACT
GGCTTTACCGGCGACTTCGACTCTGTGATAGACTGCAACACGTGTGTCACTCAGACAGTCGATTTTAGCCTTGACCCT
ACCTTTACCATTGAGACAACCACGCTCCCCCAGGATGCTGTCTCCAGGACTCAACGCCGGGGCAGGACTGGCAGGGGG
AAGCCAGGCATCTATAGATTTGTGGCACCGGGGGAGCGCCCCTCCGGCATGTTCGACTCGTCCGTCCTCTGTGAGTGC
TATGACGCGGGCTGTGCTTGGTATGAGCTCACGCCCGCCGAGACTACAGTTAGGCTACGAGCGTACATGAACACCCCG
GGGCTTCCCGTGTGCCAGGACCATCTTGGATTTTGGGAGGGCGTCTTTACGGGCCTCACTCATATAGATGCCCACTTT
CTATCCCAGACAAAGCAGAGTGGGGAGAACTTTCCTTACCTGGTAGCGTACCAAGCCACCGTGTGCGCTAGGGCTCAA
GCCCCTCCCCCATCGTGGGACCAGATGCGGAAGTGTTTGATCCGCCTTAAACCCACCCTCCATGGGCCAACACCCCTG
CTATACAGACTGGGCGCTGTTCAGAATGAAGTCACCCTGACGCACCCAATCACCAAATACATCATGACATGCATGTCG
GCCGACCTGGAGGTCGTCACG

Accordingly, preferably the HCV NS3 polypeptide is encoded by the DNA nucleotide sequence substantially as set out in SEQ ID NO: 368, or a variant or fragment thereof.

One embodiment of codon optimised nucleic acid (DNA) encoding the HCV NS3 polypeptide is provided herein as SEQ ID No: 369, as follows:

[SEQ ID No: 369]
GCCCCTATCACAGCCTACGCTCAGCAGACAAGAGGCCTGCTGGGCTGCATCATCACAAGCCTGACCGGCAGAGACAAG
AACCAGGTGGAAGGCGAGGTGCAGATCGTGTCTACAGCTACCCAGACCTTCCTGGCCACCTGTATCAATGGCGTGTGC
TGGACCGTGTATCACGGCGCTGGCACCAGAACAATCGCCTCTCCAAAGGGCCCTGTGATCCAGACCTACACCAACGTG
GACCAGGATCTCGTTGGCTGGCCTGCTCCTCAGGGCAGCAGATCTCTGACCCCTTGTACATGCGGCAGCAGCGACCTG
TACCTGGTCACAAGACACGCCGACGTGATCCCCGTCAGAAGAAGAGGCGATAGCAGAGGCAGCCTGCTGAGCCCTAGA
CCTATCAGCTACCTGAAGGGCAGCTCTGGCGGACCTCTGCTGTGTCCAACAGGACATGCCGTGGGCCTGTTTAGAGCC
GCCGTGTGTACAAGAGGCGTGGCCAAAGCCGTGGACTTCATCCCCGTGGAAAACCTGGAAACCACCATGCGGAGCCCC
GTGTTCACCGACAATTCTAGCCCTCCAGCCGTGCCTCAGAGCTTCCAAGTGGCTCATCTGCATGCCCCTACAGGCAGC
GGCAAGAGCACAAAAGTGCCTGCCGCCTATGCCGCCAAGGGCTATAAGGTGCTGGTGCTGAATCCCAGCGTGGCCGCC
ACACTTGGCTTTGGCGCCTATATGTCTAAAGCCCACGGCGTGGACCCCAACATCAGAACCGGCGTGCGGACAATCACA
ACAGGCAGCCCTATCACCTACTCTACCTACGGCAAGTTCCTGGCCGATGCCGGATGTTCTGGCGGAGCCTACGACATC
ATCATCTGCGACGAGTGCCACAGCACCGACGCCACATCTATCTCTGGCATCGGCACCGTGCTGGATCAGGCCGAAACA
GCTGGTGCTAGACTGGTGGTGCTGGCCACAGCTACACCTCCAGGCTCTGTGACAGTGTCTCACCCCAATATCGAGGAA
GTGGCCCTGTCTACAACCGGCGAGATCCCATTCTATGGCAAGGCCATTCCTCTGGAAGTGATCAAAGGCGGCAGACAC
CTGATCTTTTGCCACTCCAAGAAGAAGTGCGACGAGCTGGCCGCCAAACTGGTGGCCCTTGGAATCAATGCCGTGGCC
TACTACAGAGGACTGGACGTGTCCGTGATTCCCACATCTGGCGACGTGGTGGTGGTGTCCACTGATGCCCTGATGACC
GGCTTCACCGGCGACTTCGATAGCGTGATCGACTGCAATACCTGCGTGACCCAGACCGTGGATTTCTCTCTGGACCCC
ACCTTCACCATCGAGACAACCACACTGCCTCAGGACGCCGTGTCTCGGACACAGAGAAGAGGCAGAACCGGAAGAGGC
AAGCCCGGCATCTACAGATTTGTGGCCCCTGGCGAAAGACCCAGCGGCATGTTTGATAGCAGCGTGCTGTGCGAGTGC
TACGATGCTGGCTGTGCTTGGTACGAGCTGACCCCTGCCGAGACTACCGTTAGACTGCGGGCCTACATGAACACCCCT
GGCCTGCCTGTGTGTCAGGACCACCTCGGATTTTGGGAGGGCGTGTTCACAGGACTGACCCACATCGACGCCCACTTT
CTGAGCCAGACAAAGCAGAGCGGCGAGAACTTCCCTTACCTGGTGGCTTACCAGGCCACCGTGTGTGCTAGAGCACAA
GCCCCTCCACCTAGCTGGGACCAGATGAGGAAGTGCCTGATCCGGCTGAAGCCTACACTGCACGGACCAACACCACTG
CTGTATAGACTGGGCGCCGTGCAGAACGAAGTGACCCTGACACATCCCATCACCAAGTACATCATGACCTGCATGAGC
GCCGACCTGGAAGTGGTCACA

Hence, preferably the RNA construct is encoded by a DNA sequence substantially as set out in SEQ ID No: 369, or a fragment or variant thereof.

In an embodiment, the RNA sequence corresponding to the codon optimised DNA sequence of SEQ ID No: 369 is provided herein as SEQ ID No: 370, as follows:

[SEQ ID No: 370]
GCCCCUAUCACAGCCUACGCUCAGCAGACAAGAGGCCUGCUGGGCUGCAUCAUCACAAGCCUGACCGGCAGAGACAAG
AACCAGGUGGAAGGCGAGGUGCAGAUCGUGUCUACAGCUACCCAGACCUUCCUGGCCACCUGUAUCAAUGGCGUGUGC
UGGACCGUGUAUCACGGCGCUGGCACCAGAACAAUCGCCUCUCCAAAGGGCCCUGUGAUCCAGACCUACACCAACGUG
GACCAGGAUCUCGUUGGCUGGCCUGCUCCUCAGGGCAGCAGAUCUCUGACCCCUUGUACAUGCGGCAGCAGCGACCUG
UACCUGGUCACAAGACACGCCGACGUGAUCCCCGUCAGAAGAAGAGGCGAUAGCAGAGGCAGCCUGCUGAGCCCUAGA
CCUAUCAGCUACCUGAAGGGCAGCUCUGGCGGACCUCUGCUGUGUCCAACAGGACAUGCCGUGGGCCUGUUUAGAGCC
GCCGUGUGUACAAGAGGCGUGGCCAAAGCCGUGGACUUCAUCCCCGUGGAAAACCUGGAAACCACCAUGCGGAGCCCC
GUGUUCACCGACAAUUCUAGCCCUCCAGCCGUGCCUCAGAGCUUCCAAGUGGCUCAUCUGCAUGCCCCUACAGGCAGC
GGCAAGAGCACAAAAGUGCCUGCCGCCUAUGCCGCCAAGGGCUAUAAGGUGCUGGUGCUGAAUCCCAGCGUGGCCGCC
ACACUUGGCUUUGGCGCCUAUAUGUCUAAAGCCCACGGCGUGGACCCCAACAUCAGAACCGGCGUGCGGACAAUCACA
ACAGGCAGCCCUAUCACCUACUCUACCUACGGCAAGUUCCUGGCCGAUGCCGGAUGUUCUGGCGGAGCCUACGACAUC
AUCAUCUGCGACGAGUGCCACAGCACCGACGCCACAUCUAUCUCUGGCAUCGGCACCGUGCUGGAUCAGGCCGAAACA
GCUGGUGCUAGACUGGUGGUGCUGGCCACAGCUACACCUCCAGGCUCUGUGACAGUGUCUCACCCCAAUAUCGAGGAA
GUGGCCCUGUCUACAACCGGCGAGAUCCCAUUCUAUGGCAAGGCCAUUCCUCUGGAAGUGAUCAAAGGCGGCAGACAC
CUGAUCUUUUGCCACUCCAAGAAGAAGUGCGACGAGCUGGCCGCCAAACUGGUGGCCCUUGGAAUCAAUGCCGUGGCC
UACUACAGAGGACUGGACGUGUCCGUGAUUCCCACAUCUGGCGACGUGGUGGUGGUGUCCACUGAUGCCCUGAUGACC
GGCUUCACCGGCGACUUCGAUAGCGUGAUCGACUGCAAUACCUGCGUGACCCAGACCGUGGAUUUCUCUCUGGACCCC
ACCUUCACCAUCGAGACAACCACACUGCCUCAGGACGCCGUGUCUCGGACACAGAGAAGAGGCAGAACCGGAAGAGGC
AAGCCCGGCAUCUACAGAUUUGUGGCCCCUGGCGAAAGACCCAGCGGCAUGUUUGAUAGCAGCGUGCUGUGCGAGUGC
UACGAUGCUGGCUGUGCUUGGUACGAGCUGACCCCUGCCGAGACUACCGUUAGACUGCGGGCCUACAUGAACACCCCU
GGCCUGCCUGUGUGUCAGGACCACCUCGGAUUUUGGGAGGGCGUGUUCACAGGACUGACCCACAUCGACGCCCACUUU
CUGAGCCAGACAAAGCAGAGCGGCGAGAACUUCCCUUACCUGGUGGCUUACCAGGCCACCGUGUGUGCUAGAGCACAA
GCCCCUCCACCUAGCUGGGACCAGAUGAGGAAGUGCCUGAUCCGGCUGAAGCCUACACUGCACGGACCAACACCACUG
CUGUAUAGACUGGGCGCCGUGCAGAACGAAGUGACCCUGACACAUCCCAUCACCAAGUACAUCAUGACCUGCAUGAGC
GCCGACCUGGAAGUGGUCACA

Hence, preferably the RNA construct comprises a sequence substantially as set out in SEQ ID No: 370, or a fragment or variant thereof.

In one embodiment, the at least one IIP is DENV 1 NS4A protein (P17763; Genome polyprotein Dengue virus type 1 (strain Nauru/West Pac/1974)), or an orthologue thereof. One embodiment of the polypeptide sequence of DENV 1 NS4A protein is represented herein as SEQ ID No: 371, as follows:

[SEQ ID No: 371]
SVSGDLILEIGKLPQHLTQRAQNALDNLVMLHNSEQGGKAYRHAMEELPDTIETLMLLALIAVLTGGVTLFFLSGRGL
GKTSIGLLCVIASSALLWMASVEPHWIAASIILEFFLMVLLIPEPDRQR

Therefore, preferably the RNA construct comprises a nucleotide sequence which encodes an amino acid sequence substantially as set out in SEQ ID No: 371, or a variant or fragment thereof.

In one embodiment, the DENV 1 NS4A polypeptide is encoded by the DNA nucleotide sequence of SEQ ID No: 372, as follows:

[SEQ ID No: 372]
AGCGTCTCAGGTGACCTAATATTAGAAATAGGGAAACTTCCACAACATTTAACGCAAAGGGCCCAGAACGCCTTGGAC
AATCTGGTTATGTTGCACAACTCTGAACAAGGAGGAAAAGCCTATAGACACGCCATGGAAGAACTACCAGACACCATA
GAAACGTTAATGCTCCTAGCTTTGATAGCTGTGCTGACTGGTGGAGTGACGTTGTTCTTCCTATCAGGAAGGGGTCTA
GGAAAAACATCCATTGGCCTACTCTGCGTGATTGCCTCAAGCGCACTGCTATGGATGGCCAGTGTGGAACCCCATTGG
ATAGCGGCCTCTATCATACTGGAGTTCTTTCTGATGGTGTTGCTTATTCCAGAGCCGGACAGACAGCGC

Accordingly, preferably the DENV 1 NS4A polypeptide is encoded by the DNA nucleotide sequence substantially as set out in SEQ ID NO: 372, or a variant or fragment thereof.

One embodiment of codon optimised nucleic acid (DNA) encoding the DENV 1 NS4A polypeptide is provided herein as SEQ ID No: 373, as follows:

[SEQ ID No: 373]
TCCGTTAGCGGCGACCTGATCCTGGAAATCGGCAAGCTGCCTCAGCACCTGACACAGAGAGCACAGAACGCCCTGGAC
AACCTGGTCATGCTGCACAACTCTGAGCAAGGCGGCAAGGCCTACAGACACGCCATGGAAGAACTGCCCGACACCATC
GAGACACTGATGCTGCTGGCCCTGATCGCTGTTCTGACAGGCGGAGTGACCCTGTTCTTCCTGTCTGGCAGAGGCCTG
GGCAAGACCTCTATCGGACTGCTGTGTGTGATCGCCAGCTCTGCCCTGCTGTGGATGGCTTCTGTGGAACCTCATTGG
ATCGCCGCCTCTATTATCCTGGAATTCTTCCTGATGGTGCTGCTGATCCCCGAGCCTGACAGACAGAGA

Hence, preferably the RNA construct is encoded by a DNA sequence substantially as set out in SEQ ID No: 373, or a fragment or variant thereof.

In an embodiment, the RNA sequence corresponding to the codon optimised DNA sequence of SEQ ID No: 373 is provided herein as SEQ ID No: 374, as follows:

[SEQ ID No: 374]
UCCGUUAGCGGCGACCUGAUCCUGGAAAUCGGCAAGCUGCCUCAGCACCUGACACAGAGAGCACAGAACGCCCUGGAC
AACCUGGUCAUGCUGCACAACUCUGAGCAAGGCGGCAAGGCCUACAGACACGCCAUGGAAGAACUGCCCGACACCAUC
GAGACACUGAUGCUGCUGGCCCUGAUCGCUGUUCUGACAGGCGGAGUGACCCUGUUCUUCCUGUCUGGCAGAGGCCUG
GGCAAGACCUCUAUCGGACUGCUGUGUGUGAUCGCCAGCUCUGCCCUGCUGUGGAUGGCUUCUGUGGAACCUCAUUGG
AUCGCCGCCUCUAUUAUCCUGGAAUUCUUCCUGAUGGUGCUGCUGAUCCCCGAGCCUGACAGACAGAGA

Hence, preferably the RNA construct comprises a sequence substantially as set out in SEQ ID No: 374, or a fragment or variant thereof.

In one embodiment, the at least one IIP is DENV 1 NS2A (P17763; Genome polyprotein Dengue virus type 1 (strain Nauru/West Pac/1974)), or an orthologue thereof. One embodiment of the polypeptide sequence of DENV 1 NS2A is represented herein as SEQ ID No: 375, as follows:

[SEQ ID No: 375]
GSGEVDSFSLGLLCISIMIEEVMRSRWSRKMLMTGTLAVFLLLTMGQLTWNDLIRLCIMVGANASDKMGMGTTYLALM
ATFRMRPMFAVGLLFRRLTSREVLLLTVGLSLVASVELPNSLEELGDGLAMGIMMLKLLTDFQSHQLWATLLSLTFVK
TTFSLHYAWKTMAMILSIVSLFPLCLSTTSQKTTWLPVLLGSLGCKPLTMFLITENKIWGRK

Therefore, preferably the RNA construct comprises a nucleotide sequence which encodes an amino acid sequence substantially as set out in SEQ ID No: 375, or a variant or fragment thereof.

In one embodiment, the DENV 1 NS2A polypeptide is encoded by the DNA nucleotide sequence of SEQ ID No: 376, as follows:

[SEQ ID No: 376]
GGGTCAGGAGAAGTGGACAGTTTTTCACTAGGACTGCTATGCATATCAATAATGATCGAAGAGGTAATGAGATCCAGA
TGGAGCAGAAAAATGCTGATGACTGGAACATTGGCTGTGTTCCTCCTTCTCACAATGGGACAATTGACATGGAATGAT
CTGATCAGGCTATGTATCATGGTTGGAGCCAACGCTTCAGACAAGATGGGGATGGGAACAACGTACCTAGCTTTGATG
GCCACTTTCAGAATGAGACCAATGTTCGCAGTCGGGCTACTGTTTCGCAGATTAACATCTAGAGAAGTTCTTCTTCTT
ACAGTTGGATTGAGTCTGGTGGCATCTGTAGAACTACCAAATTCCTTAGAGGAGCTAGGGGATGGACTTGCAATGGGC
ATCATGATGTTGAAATTACTGACTGATTTTCAGTCACATCAGCTATGGGCTACCTTGCTGTCTTTAACATTTGTCAAA
ACAACTTTTTCATTGCACTATGCATGGAAGACAATGGCTATGATACTGTCAATTGTATCTCTCTTCCCTTTATGCCTG
TCCACGACTTCTCAAAAAACAACATGGCTTCCGGTGTTGCTGGGATCTCTTGGATGCAAACCACTAACCATGTTTCTT
ATAACAGAAAACAAAATCTGGGGAAGGAAA

Accordingly, preferably the DENV 1 NS2A polypeptide is encoded by the DNA nucleotide sequence substantially as set out in SEQ ID NO: 376, or a variant or fragment thereof.

One embodiment of codon optimised nucleic acid (DNA) encoding the DENV 1 NS2A polypeptide is provided herein as SEQ ID No: 377, as follows:

[SEQ ID No: 377]
GGATCTGGCGAGGTGGACTCTTTTTCTCTGGGCCTGCTGTGCATCAGCATCATGATCGAGGAAGTGATGCGGAGCCGC
TGGTCCCGGAAAATGCTGATGACTGGAACCCTGGCCGTGTTCCTGCTGCTGACAATGGGACAGCTGACCTGGAACGAC
CTGATCCGGCTGTGTATCATGGTCGGAGCCAACGCCAGCGACAAGATGGGCATGGGCACAACCTATCTGGCCCTGATG
GCCACCTTCCGGATGAGGCCTATGTTTGCCGTGGGACTGCTGTTCAGAAGGCTGACCTCTAGAGAGGTGCTGCTGCTC
ACAGTGGGCCTGTCTCTGGTGGCTTCTGTGGAACTGCCCAACAGCCTGGAAGAACTCGGAGATGGACTGGCCATGGGC
ATTATGATGCTCAAGCTGCTGACCGACTTCCAGAGCCACCAGCTGTGGGCTACACTGCTGAGCCTGACCTTCGTGAAA
ACCACCTTCAGCCTGCACTACGCCTGGAAAACAATGGCCATGATCCTGAGCATCGTGTCTCTGTTCCCTCTGTGCCTG
AGCACCACCAGCCAGAAAACCACATGGCTGCCTGTGCTGCTGGGCTCTCTGGGCTGTAAACCCCTGACCATGTTCCTG
ATCACCGAGAACAAGATCTGGGGCAGAAAG

Hence, preferably the RNA construct is encoded by a DNA sequence substantially as set out in SEQ ID No: 377, or a fragment or variant thereof.

In an embodiment, the RNA sequence corresponding to the codon optimised DNA sequence of SEQ ID No: 377 is provided herein as SEQ ID No: 378, as follows:

[SEQ ID No: 378]
GGAUCUGGCGAGGUGGACUCUUUUUCUCUGGGCCUGCUGUGCAUCAGCAUCAUGAUCGAGGAAGUGAUGCGGAGCCGC
UGGUCCCGGAAAAUGCUGAUGACUGGAACCCUGGCCGUGUUCCUGCUGCUGACAAUGGGACAGCUGACCUGGAACGAC
CUGAUCCGGCUGUGUAUCAUGGUCGGAGCCAACGCCAGCGACAAGAUGGGCAUGGGCACAACCUAUCUGGCCCUGAUG
GCCACCUUCCGGAUGAGGCCUAUGUUUGCCGUGGGACUGCUGUUCAGAAGGCUGACCUCUAGAGAGGUGCUGCUGCUC
ACAGUGGGCCUGUCUCUGGUGGCUUCUGUGGAACUGCCCAACAGCCUGGAAGAACUCGGAGAUGGACUGGCCAUGGGC
AUUAUGAUGCUCAAGCUGCUGACCGACUUCCAGAGCCACCAGCUGUGGGCUACACUGCUGAGCCUGACCUUCGUGAAA
ACCACCUUCAGCCUGCACUACGCCUGGAAAACAAUGGCCAUGAUCCUGAGCAUCGUGUCUCUGUUCCCUCUGUGCCUG
AGCACCACCAGCCAGAAAACCACAUGGCUGCCUGUGCUGCUGGGCUCUCUGGGCUGUAAACCCCUGACCAUGUUCCUG
AUCACCGAGAACAAGAUCUGGGGCAGAAAG

Hence, preferably the RNA construct comprises a sequence substantially as set out in SEQ ID No: 378, or a fragment or variant thereof.

In one embodiment, the at least one IIP is DENV 2 NS2A (P29990; Genome polyprotein Dengue virus type 2 (strain Thailand/16681/1984)), or an orthologue thereof. One embodiment of the polypeptide sequence of DENV 2 NS2A is represented herein as SEQ ID No: 379, as follows:

[SEQ ID No: 379]
GHGQVDNFSLGVLGMALFLEEMLRTRVGTKHAILLVAVSFVTLIIGNMSFRDLGRVMVMVGATMTDDIGMGVTYLALL
AAFKVRPTFAAGLLLRKLTSKALMMTTIGIVLSSQSTTPETILELTDALALGMMVLKMVRNMEKYQLAVTIMAILCVP
NAVILQNAWKVSCTILAVVSVSPLFLTSSQQKTDWIPLALTIKGLNPTAIFLTTLSRISKKR

Therefore, preferably the RNA construct comprises a nucleotide sequence which encodes an amino acid sequence substantially as set out in SEQ ID No: 379, or a variant or fragment thereof.

In one embodiment, the DENV 2 NS2A polypeptide is encoded by the DNA nucleotide sequence of SEQ ID No: 380, as follows:

[SEQ ID No: 380]
GGACATGGGCAGGTCGACAACTTTTCACTAGGAGTCTTGGGAATGGCATTGTTCCTGGAGGAAATGCTTAGGACCCGA
GTAGGAACGAAACATGCAATACTACTAGTTGCAGTTTCTTTTGTGACATTGATCATAGGGAACATGTCCTTTAGAGAC
CTGGGAAGAGTAATGGTTATGGTAGGCGCCACTATGACGGATGACATAGGTATGGGCGTGACTTATCTTGCCCTACTA
GCAGCCTTCAAAGTCAGACCAACTTTTGCAGCTGGACTACTCTTGAGAAAGCTGACCTCCAAGGCATTGATGATGACT
ACTATAGGAATTGTACTCTCCTCCCAGAGCACTACACCAGAGACCATTCTTGAGTTGACTGATGCGTTAGCCTTAGGC
ATGATGGTCCTCAAAATGGTGAGAAATATGGAAAAGTATCAATTGGCAGTGACTATCATGGCTATCTTGTGCGTCCCA
AACGCAGTGATATTACAAAACGCATGGAAAGTGAGTTGCACGATATTGGCAGTGGTGTCCGTTTCCCCACTGTTCTTA
ACATCCTCACAGCAAAAAACGGATTGGATACCATTAGCGTTGACGATCAAAGGTCTCAATCCAACAGCTATTTTTCTA
ACAACCCTCTCAAGAACCAGCAAGAAAAGG

Accordingly, preferably the DENV 2 NS2A polypeptide is encoded by the DNA nucleotide sequence substantially as set out in SEQ ID NO: 380, or a variant or fragment thereof.

One embodiment of codon optimised nucleic acid (DNA) encoding the DENV 2 NS2A polypeptide is provided herein as SEQ ID No: 381, as follows:

[SEQ ID No: 381]
GGACACGGACAGGTGGACAATTTTTCCCTGGGCGTGCTCGGCATGGCCCTGTTTCTGGAAGAGATGCTGAGAACCAGA
GTGGGCACCAAGCACGCCATTCTGCTGGTGGCCGTGTCCTTCGTGACACTGATCATCGGCAACATGAGCTTCCGCGAC
CTGGGCAGAGTGATGGTCATGGTCGGAGCCACCATGACCGACGATATCGGCATGGGCGTGACCTATCTGGCTCTGCTG
GCCGCTTTTAAAGTGCGGCCTACATTTGCCGCCGGACTGCTGCTGAGAAAGCTGACATCTAAGGCCCTGATGATGACC
ACCATCGGCATCGTGCTGAGCAGCCAGAGCACCACACCTGAGACAATCCTGGAACTGACCGACGCTCTGGCCCTGGGA
ATGATGGTGCTGAAGATGGTCCGAAACATGGAAAAGTACCAGCTGGCCGTGACCATCATGGCCATCCTGTGTGTGCCC
AACGCCGTGATCCTGCAGAACGCCTGGAAGGTGTCCTGTACCATCCTGGCCGTGGTGTCTGTGTCCCCTCTGTTTCTG
ACCAGCAGCCAGCAGAAAACCGACTGGATCCCACTGGCTCTGACCATCAAGGGCCTGAATCCTACCGCCATCTTCCTG
ACCACACTGAGCCGGACCAGCAAGAAGAGA

Hence, preferably the RNA construct is encoded by a DNA sequence substantially as set out in SEQ ID No: 381, or a fragment or variant thereof.

In an embodiment, the RNA sequence corresponding to the codon optimised DNA sequence of SEQ ID No: 381 is provided herein as SEQ ID No: 382, as follows:

[SEQ ID No: 382]
GGACACGGACAGGUGGACAAUUUUUCCCUGGGCGUGCUCGGCAUGGCCCUGUUUCUGGAAGAGAUGCUGAGAACCAGA
GUGGGCACCAAGCACGCCAUUCUGCUGGUGGCCGUGUCCUUCGUGACACUGAUCAUCGGCAACAUGAGCUUCCGCGAC
CUGGGCAGAGUGAUGGUCAUGGUCGGAGCCACCAUGACCGACGAUAUCGGCAUGGGCGUGACCUAUCUGGCUCUGCUG
GCCGCUUUUAAAGUGCGGCCUACAUUUGCCGCCGGACUGCUGCUGAGAAAGCUGACAUCUAAGGCCCUGAUGAUGACC
ACCAUCGGCAUCGUGCUGAGCAGCCAGAGCACCACACCUGAGACAAUCCUGGAACUGACCGACGCUCUGGCCCUGGGA
AUGAUGGUGCUGAAGAUGGUCCGAAACAUGGAAAAGUACCAGCUGGCCGUGACCAUCAUGGCCAUCCUGUGUGUGCCC
AACGCCGUGAUCCUGCAGAACGCCUGGAAGGUGUCCUGUACCAUCCUGGCCGUGGUGUCUGUGUCCCCUCUGUUUCUG
ACCAGCAGCCAGCAGAAAACCGACUGGAUCCCACUGGCUCUGACCAUCAAGGGCCUGAAUCCUACCGCCAUCUUCCUG
ACCACACUGAGCCGGACCAGCAAGAAGAGA

Hence, preferably the RNA construct comprises a sequence substantially as set out in SEQ ID No: 382, or a fragment or variant thereof.

In one embodiment, the at least one IIP is DENV 4 NS2A (P09866; Genome polyprotein Dengue virus type 4 (strain Dominica/814669/1981)), or an orthologue thereof. One embodiment of the polypeptide sequence of DENV 4 NS2A is represented herein as SEQ ID No: 383, as follows:

[SEQ ID No: 383]
GQGTSETFSMGLLCLTLFVEECLRRRVTRKHMILVVVITLCAIILGGLTWMDLLRALIMLGDTMSGRIGGQIHLAIMA
VFKMSPGYVLGVFLRKLTSRETALMVIGMAMTTVLSIPHDLMELIDGISLGLILLKIVTQFDNTQVGTLALSLTFIRS
TMPLVMAWRTIMAVLFVVTLIPLCRTSCLQKQSHWVEITALILGAQALPVYLMTLMKGASRR

Therefore, preferably the RNA construct comprises a nucleotide sequence which encodes an amino acid sequence substantially as set out in SEQ ID No: 383, or a variant or fragment thereof.

In one embodiment, the DENV 4 NS2A polypeptide is encoded by the DNA nucleotide sequence of SEQ ID No: 384, as follows:

[SEQ ID No: 384]
GGACAGGGCACATCAGAAACTTTTTCTATGGGTCTGTTGTGCCTGACCTTGTTTGTGGAAGAATGCTTGAGGAGAAGA
TTACTACGAGCCCTCATCATGTTGGGGGACACTATGTCTGGTAGAATAGGAGGACAGATCCACCTAGCCATCATGGCA
GTGTTCAAGATGTCACCAGGATACGTGCTGGGTGTGTTTTTAAGGAAACTCACTTCAAGAGAGACAGCACTAATGGTA
ATAGGAATGGCCATGACAACGGTGCTTTCAATTCCACATGACCTTATGGAACTCATTGATGGAATATCACTGGGACTA
AGCTGTCTTCAAAAACAGTCTCATTGGGTAGAAATAACAGCACTCATCCTAGGAGCCCAAGCTCTGCCAGTGTACCTA
ATGACTCTTATGAAAGGAGCCTCAAGAAGA

Accordingly, preferably the DENV 4 NS2A polypeptide is encoded by the DNA nucleotide sequence substantially as set out in SEQ ID NO: 384, or a variant or fragment thereof.

One embodiment of codon optimised nucleic acid (DNA) encoding the DENV 4 NS2A polypeptide is provided herein as SEQ ID No: 385, as follows:

[SEQ ID No: 385]
GGCCAGGGAACAAGCGAGACATTTTCCATGGGCCTGCTGTGTCTGACCCTGTTCGTGGAAGAGTGCCTGCGGAGAAGA
GTGACCCGGAAGCACATGATCCTGGTGGTGGTCATCACCCTGTGCGCCATCATTCTCGGCGGCCTGACATGGATGGAT
CTGCTGAGAGCCCTGATCATGCTGGGCGATACCATGAGCGGCAGAATCGGCGGACAGATCCACCTGGCCATCATGGCC
GTGTTCAAGATGAGCCCTGGCTACGTGCTGGGCGTGTTCCTGAGAAAGCTGACCAGCAGAGAAACAGCCCTGATGGTC
ATCGGAATGGCCATGACCACCGTGCTGAGCATCCCTCACGACCTGATGGAACTGATCGACGGCATCAGCCTGGGCCTG
ATCCTGCTGAAGATCGTGACCCAGTTCGACAACACCCAAGTGGGCACACTGGCCCTGAGCCTGACCTTCATCAGATCC
ACAATGCCCCTCGTGATGGCCTGGCGGACAATTATGGCCGTGCTGTTCGTCGTGACACTGATCCCTCTGTGCAGAACC
AGCTGCCTGCAGAAACAGAGCCACTGGGTCGAGATCACCGCTCTGATTCTGGGAGCACAGGCCCTGCCTGTGTACCTG
ATGACACTTATGAAGGGCGCCAGCAGACGG

Hence, preferably the RNA construct is encoded by a DNA sequence substantially as set out in SEQ ID No: 385, or a fragment or variant thereof.

In an embodiment, the RNA sequence corresponding to the codon optimised DNA sequence of SEQ ID No: 385 is provided herein as SEQ ID No: 386, as follows:

[SEQ ID No: 386]
GGCCAGGGAACAAGCGAGACAUUUUCCAUGGGCCUGCUGUGUCUGACCCUGUUCGUGGAAGAGUGCCUGCGGAGAAGA
GUGACCCGGAAGCACAUGAUCCUGGUGGUGGUCAUCACCCUGUGCGCCAUCAUUCUCGGCGGCCUGACAUGGAUGGAU
CUGCUGAGAGCCCUGAUCAUGCUGGGCGAUACCAUGAGCGGCAGAAUCGGCGGACAGAUCCACCUGGCCAUCAUGGCC
GUGUUCAAGAUGAGCCCUGGCUACGUGCUGGGCGUGUUCCUGAGAAAGCUGACCAGCAGAGAAACAGCCCUGAUGGUC
AUCGGAAUGGCCAUGACCACCGUGCUGAGCAUCCCUCACGACCUGAUGGAACUGAUCGACGGCAUCAGCCUGGGCCUG
AUCCUGCUGAAGAUCGUGACCCAGUUCGACAACACCCAAGUGGGCACACUGGCCCUGAGCCUGACCUUCAUCAGAUCC
ACAAUGCCCCUCGUGAUGGCCUGGCGGACAAUUAUGGCCGUGCUGUUCGUCGUGACACUGAUCCCUCUGUGCAGAACC
AGCUGCCUGCAGAAACAGAGCCACUGGGUCGAGAUCACCGCUCUGAUUCUGGGAGCACAGGCCCUGCCUGUGUACCUG
AUGACACUUAUGAAGGGCGCCAGCAGACGG

Hence, preferably the RNA construct comprises a sequence substantially as set out in SEQ ID No: 386, or a fragment or variant thereof.

In one embodiment, the at least one IIP is DENV 1 NS2B (P17763; Genome polyprotein Dengue virus type 1 (strain Nauru/West Pac/1974)), or an orthologue thereof. One embodiment of the polypeptide sequence of DENV 1 NS2B is represented herein as SEQ ID No: 387, as follows:

[SEQ ID No: 387]
SWPLNEGIMAVGIVSILLSSLLKNDVPLAGPLIAGGMLIACYVISGSSADLSLEKAAEVSWEEEAEHSGASHNILVEV
QDDGTMKIKDEERDDTLTILLKATLLAISGVYPMSIPATLFVWYFWQKKKQR

Therefore, preferably the RNA construct comprises a nucleotide sequence which encodes an amino acid sequence substantially as set out in SEQ ID No: 387, or a variant or fragment thereof.

In one embodiment, the DENV 1 NS2B polypeptide is encoded by the DNA nucleotide sequence of SEQ ID No: 388, as follows:

[SEQ ID No: 388]
AGCTGGCCTCTCAATGAAGGAATTATGGCTGTTGGAATAGTTAGCATTCTTCTAAGTTCACTTCTCAAGAATGATGTG
CCACTAGCTGGCCCACTAATAGCTGGAGGCATGCTAATAGCATGTTATGTCATATCTGGAAGCTCGGCCGATTTATCA
CTGGAGAAAGCGGCTGAGGTCTCCTGGGAAGAAGAAGCAGAACACTCTGGTGCCTCACACAACATACTAGTGGAGGTC
CAAGATGATGGAACCATGAAGATAAAGGATGAAGAGAGAGATGACACACTCACCATTCTCCTCAAAGCAACTCTGCTA
GCAATCTCAGGGGTATACCCAATGTCAATACCGGCGACCCTCTTTGTGTGGTATTTTTGGCAGAAAAAGAAACAGAGA

Accordingly, preferably the DENV 1 NS2B polypeptide is encoded by the DNA nucleotide sequence substantially as set out in SEQ ID NO: 388, or a variant or fragment thereof.

One embodiment of codon optimised nucleic acid (DNA) encoding the DENV 1 NS2B polypeptide is provided herein as SEQ ID No: 389, as follows:

[SEQ ID No: 389]
AGCTGGCCTCTGAACGAGGGAATTATGGCCGTGGGCATCGTGTCCATCCTGCTGTCTAGCCTGCTGAAGAACGACGTG
CCACTGGCCGGACCTCTTATTGCTGGCGGAATGCTGATCGCCTGCTACGTGATCAGCGGCAGCTCTGCCGATCTGAGC
CTGGAAAAAGCCGCCGAGGTGTCCTGGGAAGAAGAGGCCGAACATTCTGGCGCCTCTCACAACATCCTGGTGGAAGTG
CAGGACGACGGCACCATGAAGATCAAGGACGAGGAACGGGACGACACCCTGACCATTCTGCTGAAGGCTACCCTGCTG
GCCATCAGCGGAGTGTACCCTATGAGCATCCCCGCCACTCTGTTCGTGTGGTACTTCTGGCAGAAGAAGAAGCAGCGG

Hence, preferably the RNA construct is encoded by a DNA sequence substantially as set out in SEQ ID No: 389, or a fragment or variant thereof.

[SEQ ID No: 390]
AGCUGGCCUCUGAACGAGGGAAUUAUGGCCGUGGGCAUCGUGUCCAUCCUGCUGUCUAGCCUGCUGAAGAACGACGUG
CCACUGGCCGGACCUCUUAUUGCUGGCGGAAUGCUGAUCGCCUGCUACGUGAUCAGCGGCAGCUCUGCCGAUCUGAGC
CUGGAAAAAGCCGCCGAGGUGUCCUGGGAAGAAGAGGCCGAACAUUCUGGCGCCUCUCACAACAUCCUGGUGGAAGUG
CAGGACGACGGCACCAUGAAGAUCAAGGACGAGGAACGGGACGACACCCUGACCAUUCUGCUGAAGGCUACCCUGCUG
GCCAUCAGCGGAGUGUACCCUAUGAGCAUCCCCGCCACUCUGUUCGUGUGGUACUUCUGGCAGAAGAAGAAGCAGCGG

Hence, preferably the RNA construct comprises a sequence substantially as set out in SEQ ID No: 390, or a fragment or variant thereof.

In one embodiment, the at least one IIP is DENV 2 NS2B (P29990; Genome polyprotein Dengue virus type 2 (strain Thailand/16681/1984)), or an orthologue thereof. One embodiment of the polypeptide sequence of DENV 2 NS2B is represented herein as SEQ ID No: 391, as follows:

[SEQ ID No: 391]
SWPLNEAIMAVGMVSILASSLLKNDIPMTGPLVAGGPLTVCYVLTGRSA
DLELERAADVKWEDQAEISGSSPILSITISEDGSMSIKNEEEEQTLTIL
IRTGLLVISGLFPVSIPITAAAWYLWEVKKQR

Therefore, preferably the RNA construct comprises a nucleotide sequence which encodes an amino acid sequence substantially as set out in SEQ ID No: 391, or a variant or fragment thereof.

In one embodiment, the DENV 2 NS2B polypeptide is encoded by the DNA nucleotide sequence of SEQ ID No: 392, as follows:

[SEQ ID No: 392]
AGCTGGCCATTAAATGAGGCTATCATGGCAGTCGGGATGGTGAGCATTTTAGCCAGTTCTCTCCTAAAAAATGATATT
CTGGAGAGAGCAGCCGATGTCAAATGGGAAGACCAGGCAGAGATATCAGGAAGCAGCCCAATCCTGTCAATAACAATA
TCAGAAGATGGTAGCATGTCGATAAAAAATGAAGAGGAAGAACAAACACTGACCATACTCATTAGAACAGGATTGCTG
GTGATCTCAGGACTTTTTCCTGTATCAATACCAATCACGGCAGCAGCATGGTACCTGTGGGAAGTGAAGAAACAACGG

Accordingly, preferably the DENV 2 NS2B polypeptide is encoded by the DNA nucleotide sequence substantially as set out in SEQ ID NO: 392, or a variant or fragment thereof.

One embodiment of codon optimised nucleic acid (DNA) encoding the DENV 2 NS2B polypeptide is provided herein as SEQ ID No: 393, as follows:

[SEQ ID No: 393]
AGCTGGCCTCTGAACGAGGCCATTATGGCCGTCGGCATGGTGTCTATCCTGGCCAGCAGCCTGCTGAAGAACGACATC
CCTATGACAGGCCCTCTGGTGGCTGGTGGACCTCTGACAGTGTGTTACGTGCTGACAGGCAGAAGCGCCGACCTGGAA
CTTGAAAGGGCCGCTGATGTGAAGTGGGAAGATCAGGCCGAGATCAGCGGCAGCAGCCCTATCCTGAGCATCACCATC
AGCGAGGACGGCAGCATGAGCATCAAGAACGAGGAAGAGGAACAGACCCTGACCATCCTGATCAGAACCGGCCTGCTG
GTCATCAGCGGACTGTTCCCTGTGTCAATCCCCATCACAGCCGCCGCTTGGTATCTGTGGGAAGTGAAGAAGCAGCGG

Hence, preferably the RNA construct is encoded by a DNA sequence substantially as set out in SEQ ID No: 393, or a fragment or variant thereof.

In an embodiment, the RNA sequence corresponding to the codon optimised DNA sequence of SEQ ID No: 393 is provided herein as SEQ ID No: 394, as follows:

[SEQ ID No: 394]
AGCUGGCCUCUGAACGAGGCCAUUAUGGCCGUCGGCAUGGUGUCUAUCCUGGCCAGCAGCCUGCUGAAGAACGACAUC
CCUAUGACAGGCCCUCUGGUGGCUGGUGGACCUCUGACAGUGUGUUACGUGCUGACAGGCAGAAGCGCCGACCUGGAA
CUUGAAAGGGCCGCUGAUGUGAAGUGGGAAGAUCAGGCCGAGAUCAGCGGCAGCAGCCCUAUCCUGAGCAUCACCAUC
AGCGAGGACGGCAGCAUGAGCAUCAAGAACGAGGAAGAGGAACAGACCCUGACCAUCCUGAUCAGAACCGGCCUGCUG
GUCAUCAGCGGACUGUUCCCUGUGUCAAUCCCCAUCACAGCCGCCGCUUGGUAUCUGUGGGAAGUGAAGAAGCAGCGG

Hence, preferably the RNA construct comprises a sequence substantially as set out in SEQ ID No: 394, or a fragment or variant thereof.

In one embodiment, the at least one IIP is DENV 4 NS2B (P09866; Genome polyprotein virus type 4 (strain Dominica/814669/1981)), or an orthologue thereof. One embodiment of the polypeptide sequence of DENV 4 NS2B is represented herein as SEQ ID No: 395, as follows:

[SEQ ID No: 395]
SWPLNEGIMAVGLVSLLGSALLKNDVPLAGPMVAGGLLLAAYVMSGSSA
DLSLEKAANVQWDEMADITGSSPIIEVKQDEDGSFSIRDVEETNMITLL
VKLALITVSGLYPLAIPVTMTLWYMWQVKTQR

Therefore, preferably the RNA construct comprises a nucleotide sequence which encodes an amino acid sequence substantially as set out in SEQ ID No: 395, or a variant or fragment thereof.

In one embodiment, the DENV 4 NS2B polypeptide is encoded by the DNA nucleotide sequence of SEQ ID No: 396, as follows:

[SEQ ID No: 396]
TCTTGGCCTCTTAACGAGGGCATAATGGCTGTGGGTTTGGTTAGTCTCTTAGGAAGCGCTCTTTTAAAGAATGATGTC
CCTTTAGCTGGCCCAATGGTGGCAGGAGGCTTACTTCTGGCGGCTTACGTGATGAGTGGTAGCTCAGCAGATCTGTCA
CTAGAGAAGGCCGCCAACGTGCAGTGGGATGAAATGGCAGACATAACAGGCTCAAGCCCAATCATAGAAGTGAAGCAG
GATGAAGATGGCTCTTTCTCCATACGGGACGTCGAGGAAACCAATATGATAACCCTTTTGGTGAAACTGGCACTGATA
ACAGTGTCAGGTCTCTACCCCTTGGCAATTCCAGTCACAATGACCTTATGGTACATGTGGCAAGTGAAAACACAAAGA

Accordingly, preferably the DENV 4 NS2B polypeptide is encoded by the DNA nucleotide sequence substantially as set out in SEQ ID NO: 396, or a variant or fragment thereof.

One embodiment of codon optimised nucleic acid (DNA) encoding the DENV 4 NS2B polypeptide is provided herein as SEQ ID No: 397, as follows:

[SEQ ID No: 397]
AGCTGGCCTCTGAACGAGGGAATCATGGCCGTTGGCCTGGTGTCTCTGCTGGGATCTGCCCTGCTGAAGAACGATGTG
CCTCTGGCCGGACCTATGGTTGCTGGTGGACTGCTGCTGGCCGCCTATGTGATGTCTGGAAGCAGCGCCGATCTGAGC
CTGGAAAAGGCCGCTAACGTGCAGTGGGACGAGATGGCCGATATCACAGGCAGCAGCCCCATCATCGAAGTGAAGCAG
GATGAGGACGGCAGCTTCAGCATCCGCGACGTGGAAGAGACAAACATGATCACCCTGCTGGTCAAGCTGGCCCTGATC
ACCGTGTCTGGCCTGTATCCTCTGGCTATCCCCGTGACCATGACACTGTGGTACATGTGGCAAGTGAAAACCCAGCGG

Hence, preferably the RNA construct is encoded by a DNA sequence substantially as set out in SEQ ID No: 397, or a fragment or variant thereof.

In an embodiment, the RNA sequence corresponding to the codon optimised DNA sequence of SEQ ID No: 397 is provided herein as SEQ ID No: 398, as follows:

[SEQ ID No: 398]
AGCUGGCCUCUGAACGAGGGAAUCAUGGCCGUUGGCCUGGUGUCUCUGCUGGGAUCUGCCCUGCUGAAGAACGAUGUG
CCUCUGGCCGGACCUAUGGUUGCUGGUGGACUGCUGCUGGCCGCCUAUGUGAUGUCUGGAAGCAGCGCCGAUCUGAGC
CUGGAAAAGGCCGCUAACGUGCAGUGGGACGAGAUGGCCGAUAUCACAGGCAGCAGCCCCAUCAUCGAAGUGAAGCAG
GAUGAGGACGGCAGCUUCAGCAUCCGCGACGUGGAAGAGACAAACAUGAUCACCCUGCUGGUCAAGCUGGCCCUGAUC
ACCGUGUCUGGCCUGUAUCCUCUGGCUAUCCCCGUGACCAUGACACUGUGGUACAUGUGGCAAGUGAAAACCCAGCGG

Hence, preferably the RNA construct comprises a sequence substantially as set out in SEQ ID No: 398, or a fragment or variant thereof.

In one embodiment, the at least one IIP is WNV NS4A (P06935; Genome polyprotein West Nile virus NS4A), or an orthologue thereof. One embodiment of the polypeptide sequence of WNV NS4A is represented herein as SEQ ID No: 399, as follows:

[SEQ ID No: 399]
SQIGLVEVLGRMPEHFMVKTWEALDTMYVVATAEKGGRAHRMALEELPD
ALQTIVLIALLSVMSLGVFFLLMQRKGIGKIGLGGVILGAATFFCWMAE
VPGTKIAGMLLLSLLLMIVLIPEPEKQR

Therefore, preferably the RNA construct comprises a nucleotide sequence which encodes an amino acid sequence substantially as set out in SEQ ID No: 399, or a variant or fragment thereof.

In one embodiment, the WNV NS4A polypeptide is encoded by the DNA nucleotide sequence of SEQ ID No: 400, as follows:

[SEQ ID No: 400]
TCACAAATCGGGCTCGTTGAGGTGCTCGGGAGAATGCCTGAACACTTCATGGTGAAAACTTGGGAGGCATTGGACACG
ATGTATGTGGTGGCGACCGCTGAAAAAGGAGGCCGAGCTCACAGGATGGCTCTTGAGGAGCTACCGGACGCCCTTCAG
ACAATAGTTTTGATTGCACTATTGAGTGTGATGTCCTTAGGTGTGTTTTTTCTACTCATGCAAAGGAAGGGCATTGGT
AAGATTGGCTTGGGAGGAGTAATCTTAGGAGCTGCCACATTCTTCTGCTGGATGGCTGAAGTCCCAGGAACGAAAATA
GCAGGCATGCTCCTGCTTTCCCTGCTGCTCATGATTGTTTTGATTCCGGAGCCGGAAAAGCAGCGC

Accordingly, preferably the WNV NS4A polypeptide is encoded by the DNA nucleotide sequence substantially as set out in SEQ ID NO: 400, or a variant or fragment thereof.

One embodiment of codon optimised nucleic acid (DNA) encoding the WNV NS4A polypeptide is provided herein as SEQ ID No: 401, as follows:

[SEQ ID No: 401]
TCTCAGATCGGCCTGGTGGAAGTGCTGGGCAGAATGCCTGAGCACTTCATGGTCAAGACCTGGGAAGCCCTGGACACT
ATGTACGTGGTGGCCACAGCCGAGAAAGGCGGCAGAGCACATAGAATGGCCCTGGAAGAACTGCCCGACGCTCTGCAG
ACAATCGTGCTGATTGCCCTGCTGAGCGTGATGAGCCTGGGCGTGTTCTTCCTGCTGATGCAGAGAAAAGGCATCGGC
AAGATCGGACTCGGCGGCGTTATACTGGGAGCCGCCACCTTCTTTTGCTGGATGGCTGAAGTGCCCGGCACCAAGATT
GCCGGAATGCTGCTGCTGTCCCTGCTGCTGATGATTGTGCTGATCCCCGAGCCTGAGAAGCAGAGA

Hence, preferably the RNA construct is encoded by a DNA sequence substantially as set out in SEQ ID No: 401, or a fragment or variant thereof.

In an embodiment, the RNA sequence corresponding to the codon optimised DNA sequence of SEQ ID No: 401 is provided herein as SEQ ID No: 402, as follows:

[SEQ ID No: 402]
UCUCAGAUCGGCCUGGUGGAAGUGCUGGGCAGAAUGCCUGAGCACUUCAUGGUCAAGACCUGGGAAGCCCUGGACACU
AUGUACGUGGUGGCCACAGCCGAGAAAGGCGGCAGAGCACAUAGAAUGGCCCUGGAAGAACUGCCCGACGCUCUGCAG
ACAAUCGUGCUGAUUGCCCUGCUGAGCGUGAUGAGCCUGGGCGUGUUCUUCCUGCUGAUGCAGAGAAAAGGCAUCGGC
AAGAUCGGACUCGGCGGCGUUAUACUGGGAGCCGCCACCUUCUUUUGCUGGAUGGCUGAAGUGCCCGGCACCAAGAUU
GCCGGAAUGCUGCUGCUGUCCCUGCUGCUGAUGAUUGUGCUGAUCCCCGAGCCUGAGAAGCAGAGA

Hence, preferably the RNA construct comprises a sequence substantially as set out in SEQ ID No: 402, or a fragment or variant thereof.

In one embodiment, the at least one IIP is WNV NS4B (P06935; Genome polyprotein West Nile virus NS4A), or an orthologue thereof. One embodiment of the polypeptide sequence of WNV NS4B is represented herein as SEQ ID No: 403, as follows:

[SEQ ID No: 403]
NEMGWLDKTKNDIGSLLGHRPEARETTLGVESFLLDLRPATAWSLYAVTTAVLTPLLKHLITSDYINTSLTSINVQAS
ALFTLARGFPFVDVGVSALLLAVGCWGQVTLTVTVTAAALLFCHYAYMVPGWQAEAMRSAQRRTAAGIMKNVVVDGIV
ATDVPELERTTPVMQKKVGQIILILVSMAAVVVNPSVRTVREAGILTTAAAVTLWENGASSVWNATTAIGLCHIMRGG
WLSCLSIMWTLIKNMEKPGLKR

Therefore, preferably the RNA construct comprises a nucleotide sequence which encodes an amino acid sequence substantially as set out in SEQ ID No: 403, or a variant or fragment thereof.

In one embodiment, the WNV NS4B polypeptide is encoded by the DNA nucleotide sequence of SEQ ID No: 404, as follows:

[SEQ ID No: 404]
AATGAAATGGGCTGGCTGGACAAGACCAAGAATGACATTGGCAGCCTGTTGGGGCACAGGCCAGAAGCTAGAGAGACG
ACCCTGGGAGTTGAGAGCTTCTTACTTGATCTGCGGCCGGCCACGGCATGGTCGCTCTATGCCGTAACGACAGCCGTT
CTCACCCCTTTGCTGAAGCATCTAATCACGTCAGACTACATCAACACTTCGTTGACCTCAATAAACGTCCAAGCCAGC
GCGTTGTTCACTTTGGCCAGAGGCTTCCCTTTTGTGGACGTTGGTGTGTCAGCTCTCTTGCTGGCGGTCGGGTGCTGG
GGTCAGGTGACTCTGACTGTGACTGTGACTGCAGCTGCTCTGCTCTTTTGCCACTATGCTTACATGGTGCCAGGCTGG
CAAGCGGAAGCCATGCGATCTGCCCAGCGGCGGACAGCTGCTGGCATCATGAAAAATGTAGTGGTGGATGGGATCGTG
GCCACTGATGTACCTGAACTTGAACGAACAACTCCAGTCATGCAGAAAAAAGTTGGACAGATCATATTGATCTTGGTA
TCAATGGCCGCGGTGGTCGTCAATCCATCAGTGAGAACCGTCAGAGAGGCCGGAATTCTGACTACAGCAGCAGCAGTC
ACCCTATGGGAGAATGGTGCTAGTTCAGTGTGGAATGCAACGACAGCTATTGGCCTTTGTCACATCATGCGAGGAGGA
TGGCTCTCGTGTCTCTCCATCATGTGGACTCTCATCAAAAACATGGAGAAACCAGGCCTCAAGAGG

Accordingly, preferably the WNV NS4B polypeptide is encoded by the DNA nucleotide sequence substantially as set out in SEQ ID NO: 404, or a variant or fragment thereof.

One embodiment of codon optimised nucleic acid (DNA) encoding the WNV NS4B polypeptide is provided herein as SEQ ID No: 405, as follows:

[SEQ ID No: 405]
AACGAGATGGGCTGGCTGGACAAGACCAAGAACGACATCGGAAGCCTGCTGGGCCACAGACCTGAGGCCAGAGAAACA
ACCCTGGGCGTCGAGAGCTTCCTGCTGGATCTTAGACCTGCCACCGCTTGGAGCCTGTACGCCGTTACAACAGCCGTG
CTGACCCCTCTGCTGAAGCACCTGATCACCAGCGACTACATCAACACCAGCCTGACCAGCATCAACGTGCAGGCCAGC
GCTCTGTTTACCCTGGCCAGAGGCTTCCCCTTTGTGGACGTGGGAGTTTCTGCTCTGCTGCTGGCCGTTGGCTGTTGG
GGACAAGTGACCCTGACCGTGACAGTGACTGCTGCCGCTCTGCTGTTCTGCCACTACGCCTATATGGTGCCTGGATGG
CAGGCCGAGGCCATGAGATCTGCCCAGAGAAGAACAGCCGCCGGAATCATGAAGAACGTGGTGGTGGATGGCATCGTG
GCCACCGACGTTCCAGAGCTGGAAAGAACCACACCTGTGATGCAGAAGAAAGTCGGCCAGATCATCCTGATCCTGGTG
TCCATGGCCGCCGTGGTGGTCAATCCTAGCGTGCGGACAGTTAGAGAGGCCGGCATCCTGACAACAGCTGCCGCTGTT
ACCCTGTGGGAGAATGGCGCTAGCAGCGTGTGGAATGCCACCACAGCCATCGGCCTGTGCCACATCATGAGAGGCGGC
TGGCTGAGCTGCCTGAGCATCATGTGGACCCTGATCAAGAACATGGAAAAGCCCGGCCTGAAGCGG

Hence, preferably the RNA construct is encoded by a DNA sequence substantially as set out in SEQ ID No: 405, or a fragment or variant thereof.

In an embodiment, the RNA sequence corresponding to the codon optimised DNA sequence of SEQ ID No: 405 is provided herein as SEQ ID No: 406, as follows:

[SEQ ID No: 406]
AACGAGAUGGGCUGGCUGGACAAGACCAAGAACGACAUCGGAAGCCUGCUGGGCCACAGACCUGAGGCCAGAGAAACA
ACCCUGGGCGUCGAGAGCUUCCUGCUGGAUCUUAGACCUGCCACCGCUUGGAGCCUGUACGCCGUUACAACAGCCGUG
CUGACCCCUCUGCUGAAGCACCUGAUCACCAGCGACUACAUCAACACCAGCCUGACCAGCAUCAACGUGCAGGCCAGC
GCUCUGUUUACCCUGGCCAGAGGCUUCCCCUUUGUGGACGUGGGAGUUUCUGCUCUGCUGCUGGCCGUUGGCUGUUGG
GGACAAGUGACCCUGACCGUGACAGUGACUGCUGCCGCUCUGCUGUUCUGCCACUACGCCUAUAUGGUGCCUGGAUGG
CAGGCCGAGGCCAUGAGAUCUGCCCAGAGAAGAACAGCCGCCGGAAUCAUGAAGAACGUGGUGGUGGAUGGCAUCGUG
GCCACCGACGUUCCAGAGCUGGAAAGAACCACACCUGUGAUGCAGAAGAAAGUCGGCCAGAUCAUCCUGAUCCUGGUG
UCCAUGGCCGCCGUGGUGGUCAAUCCUAGCGUGCGGACAGUUAGAGAGGCCGGCAUCCUGACAACAGCUGCCGCUGUU
ACCCUGUGGGAGAAUGGCGCUAGCAGCGUGUGGAAUGCCACCACAGCCAUCGGCCUGUGCCACAUCAUGAGAGGCGGC
UGGCUGAGCUGCCUGAGCAUCAUGUGGACCCUGAUCAAGAACAUGGAAAAGCCCGGCCUGAAGCGG

Hence, preferably the RNA construct comprises a sequence substantially as set out in SEQ ID No: 406, or a fragment or variant thereof.

In one embodiment, the at least one IIP is Rabies Virus PP (P69479; Phosphoprotein Rabies virus (strain ERA)), or an orthologue thereof. One embodiment of the polypeptide sequence of Rabies Virus PP is represented herein as SEQ ID No: 407, as follows:

[SEQ ID No: 407]
MSKIFVNPSAIRAGLADLEMAEETVDLINRNIEDNQAHLQGEPIEVDNLPEDMGRLHLDDGKSPNPGEMAKVGEGKYR
EDFQMDEGEDPSFLFQSYLENVGVQIVRQMRSGERFLKIWSQTVEEIISYVAVNFPNPPGKSSEDKSTQTTGRELKKE
TTPTPSQRESQSSKARMAAQTASGPPALEWSATNEKDDLSVEAEIAHQIAESFSKKYKFPSRSSGILLYNFEQLKMNL
DDIVKEAKNVPGVTRLAHDGSKLPLRCVLGWVALANSKKFQLLVESDKLSKIMQDDLNRYTSC

Therefore, preferably the RNA construct comprises a nucleotide sequence which encodes an amino acid sequence substantially as set out in SEQ ID No: 407, or a variant or fragment thereof.

In one embodiment, the Rabies Virus PP polypeptide is encoded by the DNA nucleotide sequence of SEQ ID No: 408, as follows:

[SEQ ID No: 408]
ATGAGCAAGATCTTTGTCAATCCTAGTGCTATTAGAGCCGGTCTGGCCGATCTTGAGATGGCTGAAGAAACTGTTGAT
CTGATCAATAGAAATATCGAAGACAATCAGGCTCATCTCCAAGGGGAACCCATAGAAGTGGACAATCTCCCTGAGGAT
ATGGGGCGACTTCACCTGGATGATGGAAAATCGCCCAACCCTGGTGAGATGGCCAAGGTGGGAGAAGGCAAGTATCGA
GAGGACTTTCAGATGGATGAAGGAGAGGATCCTAGCTTCCTGTTCCAGTCATACCTGGAAAATGTTGGAGTCCAAATA
GTCAGACAAATGAGGTCAGGAGAGAGATTTCTCAAGATATGGTCACAGACCGTAGAAGAGATTATATCCTATGTCGCG
GTCAACTTTCCCAACCCTCCAGGAAAGTCTTCAGAGGATAAATCAACCCAGACTACTGGCCGAGAGCTCAAGAAGGAG
ACAACACCCACTCCTTCTCAGAGAGAAAGCCAATCATCGAAAGCCAGGATGGCGGCTCAAACTGCTTCTGGCCCTCCA
GCCCTTGAATGGTCGGCCACCAATGAAAAGGATGATCTATCAGTGGAGGCTGAGATCGCTCACCAGATTGCAGAAAGT
TTCTCCAAAAAATATAAGTTTCCCTCTCGATCCTCAGGGATACTCTTGTATAATTTTGAGCAATTGAAAATGAACCTT
GATGATATAGTTAAAGAGGCAAAAAATGTACCAGGTGTGACCCGTTTAGCCCATGACGGGTCCAAACTCCCCCTAAGA
TGTGTACTGGGATGGGTCGCTTTGGCCAACTCTAAGAAATTCCAGTTGTTAGTCGAATCCGACAAGCTGAGTAAAATC
ATGCAAGATGACTTGAATCGCTATACATCTTGC

Accordingly, preferably the Rabies Virus PP polypeptide is encoded by the DNA nucleotide sequence substantially as set out in SEQ ID NO: 408, or a variant or fragment thereof.

One embodiment of codon optimised nucleic acid (DNA) encoding the Rabies Virus PP polypeptide is provided herein as SEQ ID No: 409, as follows:

[SEQ ID No: 409]
ATGAGCAAGATCTTCGTGAACCCCAGCGCCATCAGAGCCGGACTGGCTGATCTGGAAATGGCCGAGGAAACCGTGGAC
CTGATCAACCGGAACATCGAGGACAATCAGGCCCATCTGCAGGGCGAGCCTATCGAGGTTGACAACCTGCCTGAGGAC
ATGGGCAGACTGCACCTGGATGATGGCAAGAGCCCTAATCCTGGCGAGATGGCCAAAGTCGGCGAGGGCAAGTACCGC
GAGGACTTCCAAATGGACGAGGGCGAAGATCCCAGCTTCCTGTTCCAGTCCTACCTGGAAAACGTGGGCGTGCAGATC
GTGCGGCAGATGAGAAGCGGCGAGCGGTTCCTGAAGATCTGGTCCCAGACCGTGGAAGAGATCATCAGCTACGTGGCC
GTGAACTTCCCCAATCCTCCAGGCAAGAGCAGCGAGGACAAGAGCACACAGACCACCGGCAGAGAGCTGAAGAAAGAG
ACAACCCCTACACCTAGCCAGAGAGAGAGCCAGAGCAGCAAGGCCAGAATGGCCGCTCAGACAGCTTCTGGACCTCCT
GCACTTGAGTGGAGCGCCACCAACGAGAAGGACGACCTGTCTGTGGAAGCCGAGATCGCCCACCAGATCGCCGAGAGC
TTCAGCAAGAAGTACAAGTTCCCCAGCAGAAGCAGCGGCATCCTGCTGTACAACTTCGAGCAGCTGAAGATGAACCTG
GACGACATCGTGAAAGAGGCCAAGAACGTCCCCGGCGTGACAAGACTGGCCCACGATGGATCTAAGCTGCCCCTGAGA
TGTGTGCTCGGATGGGTTGCCCTGGCCAACAGCAAGAAATTCCAGCTGCTGGTGGAAAGCGACAAGCTGTCCAAGATC
ATGCAGGACGATCTGAACCGGTACACCAGCTGC

Hence, preferably the RNA construct is encoded by a DNA sequence substantially as set out in SEQ ID No: 409, or a fragment or variant thereof.

In an embodiment, the RNA sequence corresponding to the codon optimised DNA sequence of SEQ ID No: 409 is provided herein as SEQ ID No: 410, as follows:

[SEQ ID No: 410]
AUGAGCAAGAUCUUCGUGAACCCCAGCGCCAUCAGAGCCGGACUGGCUGAUCUGGAAAUGGCCGAGGAAACCGUGGAC
CUGAUCAACCGGAACAUCGAGGACAAUCAGGCCCAUCUGCAGGGCGAGCCUAUCGAGGUUGACAACCUGCCUGAGGAC
AUGGGCAGACUGCACCUGGAUGAUGGCAAGAGCCCUAAUCCUGGCGAGAUGGCCAAAGUCGGCGAGGGCAAGUACCGC
GAGGACUUCCAAAUGGACGAGGGCGAAGAUCCCAGCUUCCUGUUCCAGUCCUACCUGGAAAACGUGGGCGUGCAGAUC
GUGCGGCAGAUGAGAAGCGGCGAGCGGUUCCUGAAGAUCUGGUCCCAGACCGUGGAAGAGAUCAUCAGCUACGUGGCC
GUGAACUUCCCCAAUCCUCCAGGCAAGAGCAGCGAGGACAAGAGCACACAGACCACCGGCAGAGAGCUGAAGAAAGAG
ACAACCCCUACACCUAGCCAGAGAGAGAGCCAGAGCAGCAAGGCCAGAAUGGCCGCUCAGACAGCUUCUGGACCUCCU
GCACUUGAGUGGAGCGCCACCAACGAGAAGGACGACCUGUCUGUGGAAGCCGAGAUCGCCCACCAGAUCGCCGAGAGC
UUCAGCAAGAAGUACAAGUUCCCCAGCAGAAGCAGCGGCAUCCUGCUGUACAACUUCGAGCAGCUGAAGAUGAACCUG
GACGACAUCGUGAAAGAGGCCAAGAACGUCCCCGGCGUGACAAGACUGGCCCACGAUGGAUCUAAGCUGCCCCUGAGA
UGUGUGCUCGGAUGGGUUGCCCUGGCCAACAGCAAGAAAUUCCAGCUGCUGGUGGAAAGCGACAAGCUGUCCAAGAUC
AUGCAGGACGAUCUGAACCGGUACACCAGCUGC

Hence, preferably the RNA construct comprises a sequence substantially as set out in SEQ ID No: 410, or a fragment or variant thereof.

In one embodiment, the at least one IIP is PEDV N protein (Q07499; Nucleoprotein Porcine epidemic diarrhea virus (strain CV777)), or an orthologue thereof. One embodiment of the polypeptide sequence of PEDV N protein is represented herein as SEQ ID No: 411, as follows:

[SEQ ID No: 411]
MASVSFQDRGRKRVPLSLYAPLRVTNDKPLSKVLANNAVPTNKGNKDQQIGYWNEQIRWRMRRGERIEQPSNWHFYYL
GTGPHGDLRYRTRTEGVFWVAKEGAKTEPTNLGVRKASEKPIIPKFSQQLPSVVEIVEPNTPPASRANSRSRSRGNGN
NRSRSPSNNRGNNQSRGNSQNRGNNQGRGASQNRGGNNNNNNKSRNQSNNRNQSNDRGGVTSRDDLVAAVKDALKSLG
IGENPDRHKQQQKPKQEKSDNSGKNTPKKNKSRATSKERDLKDIPEWRRIPKGENSVAACFGPRGGFKNFGDAEFVEK
GVDASGYAQIASLAPNVAALLFGGNVAVRELADSYEITYNYKMTVPKSDPNVELLVSQVDAFKTGNAKLQRKKEKKNK
RETTLQQHEEAIYDDVGAPSDVTHANLEWDTAVDGGDTAVEIINEIFDTGN

Therefore, preferably the RNA construct comprises a nucleotide sequence which encodes an amino acid sequence substantially as set out in SEQ ID No: 411, or a variant or fragment thereof.

In one embodiment, the PEDV N polypeptide is encoded by the DNA nucleotide sequence of SEQ ID No: 412, as follows:

[SEQ ID No: 412]
ATGGCTTCTGTCAGCTTTCAGGATCGTGGCCGCAAACGGGTGCCATTATCTCTCTATGCCCCTCTTAGGGTTACTAAT
GACAAGCCCCTTTCTAAGGTACTTGCAAACAACGCTGTACCCACTAACAAGGGGAATAAGGACCAGCAAATTGGGTAC
TGGAATGAGCAAATTCGCTGGCGCATGCGCCGTGGTGAGCGAATTGAACAACCTTCCAATTGGCATTTCTACTACCTC
GGAACAGGACCTCACGGCGACCTCCGTTATAGGACTCGTACTGAGGGTGTTTTCTGGGTTGCTAAAGAAGGCGCAAAG
ACTGAACCCACTAATTTGGGTGTCAGAAAGGCGTCTGAAAAGCCAATCATTCCAAAATTCTCTCAACAGCTCCCCAGT
GTAGTTGAGATTGTTGAACCTAACACACCTCCTGCTTCACGTGCAAATTCGCGTAGCAGGAGTCGTGGCAATGGCAAC
AATAGGTCTAGATCTCCAAGTAACAACAGAGGCAATAACCAGTCCCGTGGTAATTCACAGAATCGTGGAAATAACCAG
GGTCGTGGAGCTTCTCAGAACAGAGGAGGCAATAATAATAACAATAACAAGTCTCGTAACCAGTCCAATAACAGGAAC
CAGTCAAATGACCGTGGTGGTGTAACATCACGCGATGATCTGGTGGCTGCTGTCAAGGATGCACTTAAATCTTTGGGT
ATTGGAGAAAATCCTGACAGGCATAAGCAACAGCAGAAGCCTAAGCAGGAAAAGTCTGACAACAGCGGCAAAAATACA
CCTAAGAAGAACAAATCCAGGGCCACTTCGAAGGAACGTGACCTCAAAGACATCCCAGAGTGGAGGAGAATTCCCAAG
GGCGAAAATAGCGTAGCAGCTTGCTTCGGACCCAGAGGGGGCTTCAAAAACTTTGGAGATGCGGAATTTGTCGAAAAA
GGTGTTGATGCGTCAGGCTATGCTCAGATCGCCAGTTTAGCACCAAATGTTGCAGCATTGCTCTTTGGTGGTAATGTG
GCTGTTCGTGAGCTAGCGGACTCTTACGAGATTACATACAACTATAAAATGACTGTGCCAAAGTCAGATCCAAATGTT
GAGCTTCTTGTTTCACAGGTGGATGCATTTAAAACTGGGAATGCAAAACTCCAGAGAAAGAAGGAAAAGAAGAACAAG
CGTGAAACCACGCTGCAGCAGCATGAAGAGGCCATCTACGATGATGTGGGTGCGCCATCTGATGTGACCCATGCCAAT
CTGGAATGGGACACAGCTGTTGATGGTGGTGATACGGCCGTTGAAATTATCAACGAGATCTTCGATACAGGAAAT

Accordingly, preferably the PEDV N polypeptide is encoded by the DNA nucleotide sequence substantially as set out in SEQ ID NO: 412, or a variant or fragment thereof.

One embodiment of codon optimised nucleic acid (DNA) encoding the PEDV N polypeptide is provided herein as SEQ ID No: 413, as follows:

[SEQ ID No: 413]
ATGGCCAGCGTCAGCTTTCAGGACCGGGGCAGAAAAAGAGTGCCCCTGTCTCTGTACGCCCCTCTGAGAGTGACCAAC
GACAAGCCCCTGAGCAAGGTGCTGGCCAACAATGCCGTGCCTACCAACAAGGGCAACAAGGACCAGCAGATCGGCTAC
TGGAACGAGCAGATCCGGTGGCGGATGAGAAGAGGCGAGAGAATCGAGCAGCCCAGCAACTGGCACTTCTACTACCTC
GGCACAGGCCCTCACGGCGACCTGAGATACAGAACCAGAACCGAGGGCGTGTTCTGGGTCGCCAAAGAGGGCGCCAAG
ACCGAGCCTACAAATCTCGGCGTCAGAAAGGCCAGCGAGAAGCCTATCATCCCCAAGTTCAGCCAGCAGCTGCCCAGC
GTGGTGGAAATCGTGGAACCCAATACTCCTCCTGCCAGCCGGGCCAACAGCAGAAGCAGATCTAGAGGCAACGGCAAC
AATCGGAGCAGAAGCCCCAGCAACAACCGGGGCAACAACCAGTCCAGAGGCAACAGCCAGAACCGCGGAAACAATCAA
GGCAGAGGCGCTAGCCAGAACAGAGGCGGCAACAACAACAATAACAACAAGAGCCGGAACCAGTCTAACAACCGCAAC
CAGAGCAACGATAGAGGCGGCGTGACCAGCAGGGATGATCTGGTGGCTGCCGTGAAGGATGCCCTGAAGTCTCTCGGC
ATCGGCGAGAACCCCGACAGACACAAGCAGCAGCAGAAACCCAAGCAAGAGAAGTCCGACAACAGCGGCAAGAACACC
CCTAAGAAGAACAAGAGCAGGGCCACCAGCAAAGAGCGGGACCTGAAGGATATTCCCGAGTGGCGGAGAATCCCCAAG
GGCGAGAATTCTGTGGCCGCCTGCTTTGGACCAAGAGGCGGCTTCAAGAATTTCGGCGACGCCGAGTTCGTGGAAAAA
GGCGTGGACGCCTCTGGCTATGCCCAGATTGCATCTCTGGCCCCTAATGTGGCTGCCCTGCTGTTTGGCGGAAACGTG
GCCGTTAGAGAGCTGGCCGATAGCTACGAGATCACCTACAACTACAAGATGACCGTGCCTAAGAGCGACCCCAACGTG
GAACTGCTGGTGTCTCAGGTGGACGCATTCAAGACCGGCAACGCCAAGCTGCAGCGCAAGAAAGAGAAGAAAAACAAG
CGCGAGACAACCCTGCAGCAGCACGAGGAAGCTATCTACGACGACGTGGGAGCCCCTTCCGATGTGACACACGCCAAC
CTGGAATGGGACACAGCAGTGGATGGCGGCGATACCGCCGTGGAAATCATCAACGAGATCTTCGACACCGGCAAC

Hence, preferably the RNA construct is encoded by a DNA sequence substantially as set out in SEQ ID No: 413, or a fragment or variant thereof.

In an embodiment, the RNA sequence corresponding to the codon optimised DNA sequence of SEQ ID No: 413 is provided herein as SEQ ID No: 414, as follows:

[SEQ ID No: 414]
AUGGCCAGCGUCAGCUUUCAGGACCGGGGCAGAAAAAGAGUGCCCCUGUCUCUGUACGCCCCUCUGAGAGUGACCAAC
GACAAGCCCCUGAGCAAGGUGCUGGCCAACAAUGCCGUGCCUACCAACAAGGGCAACAAGGACCAGCAGAUCGGCUAC
UGGAACGAGCAGAUCCGGUGGCGGAUGAGAAGAGGCGAGAGAAUCGAGCAGCCCAGCAACUGGCACUUCUACUACCUC
GGCACAGGCCCUCACGGCGACCUGAGAUACAGAACCAGAACCGAGGGCGUGUUCUGGGUCGCCAAAGAGGGCGCCAAG
ACCGAGCCUACAAAUCUCGGCGUCAGAAAGGCCAGCGAGAAGCCUAUCAUCCCCAAGUUCAGCCAGCAGCUGCCCAGC
GUGGUGGAAAUCGUGGAACCCAAUACUCCUCCUGCCAGCCGGGCCAACAGCAGAAGCAGAUCUAGAGGCAACGGCAAC
AAUCGGAGCAGAAGCCCCAGCAACAACCGGGGCAACAACCAGUCCAGAGGCAACAGCCAGAACCGCGGAAACAAUCAA
GGCAGAGGCGCUAGCCAGAACAGAGGCGGCAACAACAACAAUAACAACAAGAGCCGGAACCAGUCUAACAACCGCAAC
CAGAGCAACGAUAGAGGCGGCGUGACCAGCAGGGAUGAUCUGGUGGCUGCCGUGAAGGAUGCCCUGAAGUCUCUCGGC
AUCGGCGAGAACCCCGACAGACACAAGCAGCAGCAGAAACCCAAGCAAGAGAAGUCCGACAACAGCGGCAAGAACACC
CCUAAGAAGAACAAGAGCAGGGCCACCAGCAAAGAGCGGGACCUGAAGGAUAUUCCCGAGUGGCGGAGAAUCCCCAAG
GGCGAGAAUUCUGUGGCCGCCUGCUUUGGACCAAGAGGCGGCUUCAAGAAUUUCGGCGACGCCGAGUUCGUGGAAAAA
GGCGUGGACGCCUCUGGCUAUGCCCAGAUUGCAUCUCUGGCCCCUAAUGUGGCUGCCCUGCUGUUUGGCGGAAACGUG
GCCGUUAGAGAGCUGGCCGAUAGCUACGAGAUCACCUACAACUACAAGAUGACCGUGCCUAAGAGCGACCCCAACGUG
GAACUGCUGGUGUCUCAGGUGGACGCAUUCAAGACCGGCAACGCCAAGCUGCAGCGCAAGAAAGAGAAGAAAAACAAG
CGCGAGACAACCCUGCAGCAGCACGAGGAAGCUAUCUACGACGACGUGGGAGCCCCUUCCGAUGUGACACACGCCAAC
CUGGAAUGGGACACAGCAGUGGAUGGCGGCGAUACCGCCGUGGAAAUCAUCAACGAGAUCUUCGACACCGGCAAC

Hence, preferably the RNA construct comprises a sequence substantially as set out in SEQ ID No: 414, or a fragment or variant thereof.

In one embodiment, the at least one IIP is HSV1 ICP27 (P10238; ICP27 mRNA export factor Human herpesvirus 1 (strain 17)), or an orthologue thereof. One embodiment of the polypeptide sequence of HSV1 ICP27 is represented herein as SEQ ID No: 415, as follows:

[SEQ ID No: 415]
MATDIDMLIDLGLDLSDSDLDEDPPEPAESRRDDLESDSSGECSSSDEDMEDPHGEDGPEPILDAARPAVRPSRPEDP
GVPSTQTPRPTERQGPNDPQPAPHSVWSRLGARRPSCSPEQHGGKVARLQPPPTKAQPARGGRRGRRRGRGRGGPGAA
DGLSDPRRRAPRTNRNPGGPRPGAGWTDGPGAPHGEAWRGSEQPDPPGGQRTRGVRQAPPPLMTLAIAPPPADPRAPA
PERKAPAADTIDATTRLVLRSISERAAVDRISESFGRSAQVMHDPFGGQPFPAANSPWAPVLAGQGGPFDAETRRVSW
ETLVAHGPSLYRTFAGNPRAASTAKAMRDCVLRQENFIEALASADETLAWCKMCIHHNLPLRPQDPIIGTTAAVLDNL
ATRLRPFLQCYLKARGLCGLDELCSRRRLADIKDIASFVFVILARLANRVERGVAEIDYATLGVGVGEKMHFYLPGAC
MAGLIEILDTHRQECSSRVCELTASHIVAPPYVHGKYFYCNSLF

Therefore, preferably the RNA construct comprises a nucleotide sequence which encodes an amino acid sequence substantially as set out in SEQ ID No: 415, or a variant or fragment thereof.

In one embodiment, the HSV1 ICP27 polypeptide is encoded by the DNA nucleotide sequence of SEQ ID No: 416, as follows:

[SEQ ID No: 416]
ATGGCGACTGACATTGATATGCTAATTGACCTCGGCCTGGACCTCTCCGACAGCGATCTGGACGAGGACCCCCCCGAG
CCGGCGGAGAGCCGCCGCGACGACCTGGAATCGGACAGCAGCGGGGAGTGTTCCTCGTCGGACGAGGACATGGAAGAC
CCCCACGGAGAGGACGGACCGGAGCCGATACTCGACGCCGCTCGCCCGGCGGTCCGCCCGTCTCGTCCAGAAGACCCC
GGCGTACCCAGCACCCAGACGCCTCGTCCGACGGAGCGGCAGGGCCCCAACGATCCTCAACCAGCGCCCCACAGTGTG
TGGTCGCGCCTCGGGGCCCGGCGACCGTCTTGCTCCCCCGAGCAGCACGGGGGCAAGGTGGCCCGCCTCCAACCCCCA
CCGACCAAAGCCCAGCCTGCCCGCGGCGGACGCCGTGGGCGTCGCAGGGGTCGGGGTCGCGGTGGTCCCGGGGCTGCC
GATGGTTTGTCGGACCCCCGCCGGCGTGCCCCCAGAACCAATCGCAACCCTGGGGGACCCCGCCCCGGGGCGGGGTGG
ACGGACGGCCCCGGCGCCCCCCATGGCGAGGCGTGGCGCGGCAGTGAGCAGCCCGACCCACCCGGAGGCCAGCGGACA
CGGGGCGTGCGCCAAGCACCCCCCCCGCTAATGACGCTGGCGATTGCCCCCCCGCCCGCGGACCCCCGCGCCCCGGCC
CCGGAGCGAAAGGCGCCCGCCGCCGACACCATCGACGCCACCACGCGGTTGGTCCTGCGCTCCATCTCCGAGCGCGCG
GCGGTCGACCGCATCAGCGAGAGCTTTGGCCGCAGCGCACAGGTCATGCACGACCCCTTTGGGGGGCAGCCGTTTCCC
GCCGCGAATAGCCCCTGGGCCCCGGTGCTGGCGGGCCAAGGAGGGCCCTTTGACGCCGAGACCAGACGGGTCTCCTGG
GAAACCTTGGTCGCCCACGGCCCGAGCCTCTATCGCACTTTTGCCGGCAATCCTCGGGCCGCATCGACCGCCAAGGCC
ATGCGCGACTGCGTGCTGCGCCAAGAAAATTTCATCGAGGCGCTGGCCTCCGCCGACGAGACGCTGGCGTGGTGCAAG
ATGTGCATCCACCACAACCTGCCGCTGCGCCCCCAGGACCCCATTATCGGGACGACCGCGGCTGTGCTGGATAACCTC
GCCACGCGCCTGCGGCCCTTTCTCCAGTGCTACCTGAAGGCGCGAGGCCTGTGCGGCCTGGACGAACTGTGTTCGCGG
CGGCGTCTGGCGGACATTAAGGACATTGCATCCTTCGTGTTTGTCATTCTGGCCAGGCTCGCCAACCGCGTCGAGCGT
GGCGTCGCGGAGATCGACTACGCGACCCTTGGTGTCGGGGTCGGAGAGAAGATGCATTTCTACCTCCCCGGGGCCTGC
ATGGCGGGCCTGATCGAAATCCTAGACACGCACCGCCAGGAGTGTTCGAGTCGTGTCTGCGAGTTGACGGCCAGTCAC
ATCGTCGCCCCCCCGTACGTGCACGGCAAATATTTTTATTGCAACTCCCTGTTT

Accordingly, preferably the HSV1 ICP27 polypeptide is encoded by the DNA nucleotide sequence substantially as set out in SEQ ID NO: 416, or a variant or fragment thereof.

One embodiment of codon optimised nucleic acid (DNA) encoding the HSV1 ICP27 polypeptide is provided herein as SEQ ID No: 417, as follows:

[SEQ ID No: 417]
ATGGCCACCGACATCGACATGCTGATCGACCTGGGCCTCGACCTGAGCGACTCTGACCTGGATGAAGATCCTCCTGAG
CCTGCCGAGAGCAGAAGGGACGATCTGGAAAGCGATAGCAGCGGCGAGTGTAGCAGCAGCGACGAGGACATGGAAGAT
CCCCACGGCGAGGATGGACCTGAGCCTATTCTGGATGCCGCCAGACCTGCCGTCAGACCTTCTAGACCTGAAGATCCA
GGCGTGCCCAGCACACAGACCCCTAGACCTACAGAGAGACAGGGCCCCAACGATCCTCAGCCTGCTCCTCATAGCGTG
TGGTCTAGACTGGGAGCCAGAAGGCCTAGCTGTAGCCCTGAACAGCACGGCGGAAAAGTGGCCAGACTGCAGCCTCCT
CCAACAAAGGCTCAACCTGCTAGAGGCGGCAGACGGGGCAGAAGAAGAGGTAGAGGAAGAGGTGGACCTGGCGCCGCT
GATGGACTGTCTGATCCTAGAAGAAGGGCCCCTCGGACCAACAGAAATCCTGGCGGACCTAGACCAGGCGCCGGATGG
ACAGATGGACCAGGTGCTCCACATGGCGAGGCTTGGAGAGGATCTGAGCAGCCTGATCCTCCAGGCGGCCAAAGAACA
AGAGGCGTTAGACAGGCTCCTCCTCCTCTGATGACCCTGGCTATTGCTCCTCCACCAGCCGATCCTAGAGCACCCGCT
CCAGAAAGAAAAGCCCCTGCCGCCGATACCATCGACGCCACAACAAGACTGGTGCTGCGGAGCATCTCTGAGAGGGCC
GCTGTGGATAGAATCAGCGAGAGCTTTGGGAGAAGCGCCCAAGTGATGCACGACCCTTTTGGCGGCCAGCCTTTTCCT
GCCGCCAATTCTCCTTGGGCTCCTGTGCTTGCTGGACAAGGCGGCCCTTTTGACGCCGAGACAAGAAGAGTGTCCTGG
GAGACACTGGTGGCCCACGGACCTAGCCTGTACAGAACATTCGCCGGCAATCCAAGAGCCGCCAGCACAGCCAAAGCC
ATGAGAGACTGCGTGCTGAGACAAGAGAACTTCATCGAGGCCCTGGCCAGCGCCGATGAGACACTTGCTTGGTGCAAG
ATGTGCATCCACCACAACCTGCCTCTGAGGCCACAGGACCCTATCATCGGAACAACAGCTGCCGTGCTGGATAACCTG
GCTACCAGACTGAGGCCCTTCCTGCAGTGCTACCTGAAGGCCAGAGGACTGTGTGGCCTGGATGAGCTGTGCTCCAGA
AGAAGGCTGGCTGACATCAAGGATATCGCCAGCTTCGTGTTCGTGATTCTGGCCCGGCTGGCCAACAGAGTGGAAAGA
GGCGTGGCCGAGATCGACTATGCCACACTCGGAGTTGGCGTGGGCGAGAAGATGCACTTTTATCTGCCTGGCGCCTGC
ATGGCCGGCCTGATCGAAATTCTGGACACCCACAGACAAGAGTGCAGCTCCAGAGTGTGCGAGCTGACAGCCTCTCAC
ATTGTGGCCCCTCCATACGTGCACGGCAAGTACTTCTACTGCAACAGCCTGTTC

Hence, preferably the RNA construct is encoded by a DNA sequence substantially as set out in SEQ ID No: 417, or a fragment or variant thereof.

In an embodiment, the RNA sequence corresponding to the codon optimised DNA sequence of SEQ ID No: 417 is provided herein as SEQ ID No: 418, as follows:

[SEQ ID No: 418]
AUGGCCACCGACAUCGACAUGCUGAUCGACCUGGGCCUCGACCUGAGCGACUCUGACCUGGAUGAAGAUCCUCCUGAG
CCUGCCGAGAGCAGAAGGGACGAUCUGGAAAGCGAUAGCAGCGGCGAGUGUAGCAGCAGCGACGAGGACAUGGAAGAU
CCCCACGGCGAGGAUGGACCUGAGCCUAUUCUGGAUGCCGCCAGACCUGCCGUCAGACCUUCUAGACCUGAAGAUCCA
GGCGUGCCCAGCACACAGACCCCUAGACCUACAGAGAGACAGGGCCCCAACGAUCCUCAGCCUGCUCCUCAUAGCGUG
UGGUCUAGACUGGGAGCCAGAAGGCCUAGCUGUAGCCCUGAACAGCACGGCGGAAAAGUGGCCAGACUGCAGCCUCCU
CCAACAAAGGCUCAACCUGCUAGAGGCGGCAGACGGGGCAGAAGAAGAGGUAGAGGAAGAGGUGGACCUGGCGCCGCU
GAUGGACUGUCUGAUCCUAGAAGAAGGGCCCCUCGGACCAACAGAAAUCCUGGCGGACCUAGACCAGGCGCCGGAUGG
ACAGAUGGACCAGGUGCUCCACAUGGCGAGGCUUGGAGAGGAUCUGAGCAGCCUGAUCCUCCAGGCGGCCAAAGAACA
AGAGGCGUUAGACAGGCUCCUCCUCCUCUGAUGACCCUGGCUAUUGCUCCUCCACCAGCCGAUCCUAGAGCACCCGCU
CCAGAAAGAAAAGCCCCUGCCGCCGAUACCAUCGACGCCACAACAAGACUGGUGCUGCGGAGCAUCUCUGAGAGGGCC
GCUGUGGAUAGAAUCAGCGAGAGCUUUGGGAGAAGCGCCCAAGUGAUGCACGACCCUUUUGGCGGCCAGCCUUUUCCU
GCCGCCAAUUCUCCUUGGGCUCCUGUGCUUGCUGGACAAGGCGGCCCUUUUGACGCCGAGACAAGAAGAGUGUCCUGG
GAGACACUGGUGGCCCACGGACCUAGCCUGUACAGAACAUUCGCCGGCAAUCCAAGAGCCGCCAGCACAGCCAAAGCC
AUGAGAGACUGCGUGCUGAGACAAGAGAACUUCAUCGAGGCCCUGGCCAGCGCCGAUGAGACACUUGCUUGGUGCAAG
AUGUGCAUCCACCACAACCUGCCUCUGAGGCCACAGGACCCUAUCAUCGGAACAACAGCUGCCGUGCUGGAUAACCUG
GCUACCAGACUGAGGCCCUUCCUGCAGUGCUACCUGAAGGCCAGAGGACUGUGUGGCCUGGAUGAGCUGUGCUCCAGA
AGAAGGCUGGCUGACAUCAAGGAUAUCGCCAGCUUCGUGUUCGUGAUUCUGGCCCGGCUGGCCAACAGAGUGGAAAGA
GGCGUGGCCGAGAUCGACUAUGCCACACUCGGAGUUGGCGUGGGCGAGAAGAUGCACUUUUAUCUGCCUGGCGCCUGC
AUGGCCGGCCUGAUCGAAAUUCUGGACACCCACAGACAAGAGUGCAGCUCCAGAGUGUGCGAGCUGACAGCCUCUCAC
AUUGUGGCCCCUCCAUACGUGCACGGCAAGUACUUCUACUGCAACAGCCUGUUC

Hence, preferably the RNA construct comprises a sequence substantially as set out in SEQ ID No: 418, or a fragment or variant thereof.

In one embodiment, the at least one IIP is HSV1VP24 (F8RDC3; Capsid scaffolding protein VP24 cleavage product of UL26 Human herpesvirus 1), or an orthologue thereof. One embodiment of the polypeptide sequence of HSV1 VP24 is represented herein as SEQ ID No: 419, as follows:

[SEQ ID No: 419]
MAADAPGDRMEEPLPDRAVPIYVAGFLALYDSGDSGELALDPDTVRAALPPDNPLPINVDHRAGCEVGRVLAVVDDPR
GPFFVGLIACVQLERVLETAASAAIFERRGPPLSREERLLYLITNYLPSVSLATKRLGGEAHPDRTLFAHVALCAIGR
RLGTIVTYDTGLDAAIAPFRHLSPASREGARRLAAEAELALSGRTWAPGVEALTHTLLSTAVNNMMLRDRWSLVAERR
RQAGIAGHTYLQA

Therefore, preferably the RNA construct comprises a nucleotide sequence which encodes an amino acid sequence substantially as set out in SEQ ID No: 419, or a variant or fragment thereof.

In one embodiment, the HSV1VP24 polypeptide is encoded by the DNA nucleotide sequence of SEQ ID No: 420, as follows:

[SEQ ID No: 420]
ATGGCAGCCGATGCCCCGGGAGACAGGATGGAGGAGCCCCTGCCAGACAG
GGCCGTGCCCATTTACGTGGCTGGGTTTTTGGCCCTGTATGACAGCGGGG
ACTCGGGCGAGTTGGCATTGGATCCGGATACGGTGCGGGCGGCCCTGCCT
CCGGATAACCCACTCCCGATTAACGTGGACCACCGCGCTGGCTGCGAGGT
GGGGCGGGTGCTGGCCGTGGTCGACGACCCCCGCGGGCCGTTTTTTGTGG
GGCTGATCGCCTGCGTGCAGCTGGAGCGCGTCCTCGAGACGGCCGCCAGC
GCTGCGATTTTCGAGCGCCGCGGGCCGCCGCTCTCCCGGGAGGAGCGCCT
GTTGTACCTGATCACCAACTACCTGCCCTCGGTCTCCCTGGCCACAAAAC
GCCTGGGGGGCGAGGCGCACCCCGATCGCACGCTGTTCGCGCACGTCGCG
CTGTGCGCGATCGGGAGGCGCCTCGGCACTATCGTCACCTACGACACCGG
TCTCGACGCCGCCATCGCGCCCTTTCGCCACCTGTCGCCGGCGTCTCGCG
AGGGGGCGCGGCGACTGGCCGCCGAGGCCGAGCTCGCGCTGTCCGGACGC
ACCTGGGCGCCCGGCGTGGAGGCGCTGACCCACACGCTGCTTTCCACCGC
CGTTAACAACATGATGCTGCGGGACCGCTGGAGCCTGGTGGCCGAGCGGC
GGCGGCAGGCCGGGATTGCCGGACACACCTACCTCCAGGCG

Accordingly, preferably the HSV1 VP24 polypeptide is encoded by the DNA nucleotide sequence substantially as set out in SEQ ID NO: 420, or a variant or fragment thereof.

One embodiment of codon optimised nucleic acid (DNA) encoding the HSV1 VP24 polypeptide is provided herein as SEQ ID No: 421, as follows:

[SEQ ID No: 421]
ATGGCTGCTGATGCCCCTGGCGACAGAATGGAAGAACCCCTGCCTGATAG
AGCCGTGCCTATCTACGTGGCCGGATTTCTGGCCCTGTACGACTCTGGCG
ATTCTGGCGAACTGGCCCTGGATCCTGATACAGTCAGAGCCGCTCTGCCT
CCTGACAACCCTCTGCCAATCAACGTGGACCACAGAGCCGGCTGTGAAGT
GGGAAGAGTGCTGGCCGTGGTGGACGATCCTAGAGGCCCTTTCTTTGTGG
GCCTGATCGCCTGCGTGCAGCTGGAAAGAGTTCTGGAAACAGCCGCCAGC
GCCGCCATCTTCGAAAGAAGAGGACCTCCTCTGAGCCGGGAAGAGAGACT
GCTGTACCTGATCACCAACTACCTGCCTAGCGTGTCCCTGGCCACAAAGA
GACTTGGCGGAGAGGCCCATCCTGACAGAACCCTGTTTGCCCATGTGGCC
CTGTGTGCCATTGGTAGAAGGCTGGGCACCATCGTGACCTACGATACAGG
ACTGGACGCCGCTATCGCCCCATTCAGACATCTGAGCCCTGCCAGCAGAG
AAGGCGCCAGAAGGCTTGCTGCTGAAGCCGAACTGGCTCTGAGCGGCAGA
ACATGGGCTCCAGGTGTTGAAGCCCTGACACACACCCTGCTGAGCACCGC
CGTGAACAACATGATGCTGCGGGACAGATGGTCCCTGGTGGCCGAGAGAA
GAAGGCAGGCTGGAATTGCCGGCCACACATATCTGCAGGCT

Hence, preferably the RNA construct is encoded by a DNA sequence substantially as set out in SEQ ID No: 421, or a fragment or variant thereof.

In an embodiment, the RNA sequence corresponding to the codon optimised DNA sequence of SEQ ID No: 421 is provided herein as SEQ ID No: 422, as follows:

[SEQ ID No: 422]
AUGGCUGCUGAUGCCCCUGGCGACAGAAUGGAAGAACCCCUGCCUGAUAG
AGCCGUGCCUAUCUACGUGGCCGGAUUUCUGGCCCUGUACGACUCUGGCG
AUUCUGGCGAACUGGCCCUGGAUCCUGAUACAGUCAGAGCCGCUCUGCCU
CCUGACAACCCUCUGCCAAUCAACGUGGACCACAGAGCCGGCUGUGAAGU
GGGAAGAGUGCUGGCCGUGGUGGACGAUCCUAGAGGCCCUUUCUUUGUGG
GCCUGAUCGCCUGCGUGCAGCUGGAAAGAGUUCUGGAAACAGCCGCCAGC
GCCGCCAUCUUCGAAAGAAGAGGACCUCCUCUGAGCCGGGAAGAGAGACU
GCUGUACCUGAUCACCAACUACCUGCCUAGCGUGUCCCUGGCCACAAAGA
GACUUGGCGGAGAGGCCCAUCCUGACAGAACCCUGUUUGCCCAUGUGGCC
CUGUGUGCCAUUGGUAGAAGGCUGGGCACCAUCGUGACCUACGAUACAGG
ACUGGACGCCGCUAUCGCCCCAUUCAGACAUCUGAGCCCUGCCAGCAGAG
AAGGCGCCAGAAGGCUUGCUGCUGAAGCCGAACUGGCUCUGAGCGGCAGA
ACAUGGGCUCCAGGUGUUGAAGCCCUGACACACACCCUGCUGAGCACCGC
CGUGAACAACAUGAUGCUGCGGGACAGAUGGUCCCUGGUGGCCGAGAGAA
GAAGGCAGGCUGGAAUUGCCGGCCACACAUAUCUGCAGGCU

Hence, preferably the RNA construct comprises a sequence substantially as set out in SEQ ID No: 422, or a fragment or variant thereof.

In one embodiment, the at least one IIP is Murine CTMV M45 (Q06A28; Ribonucleoside-diphosphate reductase large subunit-like protein Murine herpesvirus 1 (strain Smith) Murine Cytomegalovirus M45 Protein), or an orthologue thereof. Fliss P, Pechenick Jowers T et al (2012) Viral Mediated Redirection of NEMO/IKKγ to Autophagosomes Curtails the Inflammatory Cascade. PLoS Pathogens 8, 2. It is believed that M45 induces proteasome-independent degradation of NEMO. One embodiment of the polypeptide sequence of Murine CTMV M45 is represented herein as SEQ ID No: 423, as follows:

[SEQ ID No: 423]
MDRQPKVYSDPDNGFFFLDVPMPDDGQGGQQTATTAAGGAFGVGGGHSVP
YVRIMNGVSGIQIGNHNAMSIASCWSPSYTDRRRRSYPKTATNAAADRVA
AAVSAANAAVNAAAAAAAAGGGGGANLLAAAVTCANQRGCCGGNGGHSLP
PTRMPKTNATAAAAPAVAGASNAKSDNNHANATSGAGSAAATPAATTPAA
TAVENRRPSPSPSTASTAPCDEGSSPRHHRPSHVSVGTQATPSTPIPIPA
PRCSTGQQQQQPQAKKLKPAKADPLLYAATMPPPASVTTAAAAAVAPESE
SSPAASAPPAAAAMATGGDDEDQSSFSFVSDDVLGEFEDLRIAGLPVRDE
MRPPTPTMTVIPVSRPFRAGRDSGRDALFDDAVESVRCYCHGILGNSRFC
ALVNEKCSEPAKERMARIRRYAADVTRCGPLALYTAIVSSANRLIQTDPS
CDLDLAECYVETASKRNAVPLSAFYRDCDRLRDAVAAFFKTYGMVVDAMA
QRITERVGPALGRGLYSTVVMMDRCGNSFQGREETPISVFARVAAALAVE
CEVDGGVSYKILSSKPVDAAQAFDAFLSALCSFAIIPSPRVLAYAGFGGS
NPIFDAVSYRAQFYSAESTINGTLHDICDMVTNGLSVSVSAADLGGDIVA
SLHILGQQCKALRPYARFKTVLRIYFDIWSVDALKIFSFILDVGREYEGL
MAFAVNTPRIFWDRYLDSSGDKMWLMFARREAAALCGLDLKSFRNVYEKM
ERDGRSAITVSPWWAVCQLDACVARGNTAVVFPHNVKSMIPENIGRPAVC
GPGVSVVSGGFVGCTPIHELCINLENCVLEGAAVESSVDVVLGLGCRFSF
KALESLVRDAVVLGNLLIDMTVRTNAYGAGKLLTLYRDLHIGVVGFHAVM
NRLGQKFADMESYDLNQRIAEFIYYTAVRASVDLCMAGADPFPKFPKSLY
AAGRFYPDLFDDDERGPRRMTKEFLEKLREDVVKHGIRNASFITGCSADE
AANLAGTTPGFWPRRDNVFLEQTPLMMTPTKDQMLDECVRSVKIEPHRLH
EEDLSCLGENRPVELPVLNSRLRQISKESATVAVRRGRSAPFYDDSDDED
EVACSETGWTVSTDAVIKMCVDRQPFVDHAQSLPVAIGFGGSSVELARHL
RRGNALGLSVGVYKCSMPPSVNYR

Therefore, preferably the RNA construct comprises a nucleotide sequence which encodes an amino acid sequence substantially as set out in SEQ ID No: 423, or a variant or fragment thereof.

In one embodiment, the Murine CTMV M45 polypeptide is encoded by the DNA nucleotide sequence of SEQ ID No: 424, as follows:

[SEQ ID No: 424]
ATGGATCGCCAGCCCAAAGTCTACTCCGACCCGGACAACGGATTCTTCTT
TTTGGATGTCCCCATGCCTGACGACGGGCAGGGAGGCCAGCAGACCGCCA
CCACCGCTGCCGGGGGAGCCTTCGGGGTGGGTGGGGGGCACAGCGTGCCC
TACGTCAGGATCATGAATGGAGTCTCTGGAATACAGATCGGAAACCATAA
TGCTATGAGCATCGCCTCTTGCTGGAGTCCCTCCTACACTGACCGACGCC
GCAGGAGCTACCCCAAGACCGCGACCAACGCGGCGGCAGACAGGGTCGCC
GCTGCCGTCTCCGCCGCCAATGCTGCTGTCAATGCTGCTGCCGCGGCTGC
TGCCGCCGGCGGGGGCGGCGGCGCTAACCTACTGGCTGCTGCTGTCACTT
GTGCAAATCAGCGAGGTTGCTGCGGAGGAAATGGGGGGCATTCCCTCCCT
CCCACCCGAATGCCGAAGACCAACGCTACCGCCGCGGCCGCTCCTGCCGT
CGCCGGTGCTTCCAACGCCAAGAGTGACAACAATCACGCTAACGCCACCT
CTGGTGCTGGATCCGCGGCCGCCACCCCCGCCGCCACCACCCCCGCCGCC
ACCGCCGTCGAAAACCGACGACCCAGCCCGAGCCCCTCTACAGCCTCGAC
TGCGCCCTGTGACGAGGGATCTTCTCCTCGCCACCATCGTCCTAGTCACG
TTAGTGTCGGCACTCAGGCGACTCCGTCGACTCCTATCCCGATTCCCGCT
CCCCGGTGCAGCACAGGCCAACAACAACAGCAACCTCAAGCCAAGAAGCT
TAAGCCCGCTAAAGCTGATCCCCTCCTGTACGCGGCGACGATGCCGCCTC
CCGCGAGCGTAACGACCGCCGCTGCCGCTGCCGTCGCCCCTGAATCCGAA
TCCTCACCTGCCGCTTCGGCACCACCAGCAGCAGCAGCGATGGCGACCGG
GGGAGACGACGAAGATCAGTCGTCTTTCTCGTTCGTGAGCGACGACGTCC
TCGGAGAATTCGAAGATCTGCGCATCGCCGGGCTCCCCGTCAGGGACGAG
ATGCGCCCCCCGACCCCGACGATGACGGTCATTCCCGTCAGCAGGCCCTT
CCGCGCGGGGCGCGACAGCGGGCGCGACGCCTTGTTTGACGACGCCGTCG
AGTCCGTGCGCTGCTACTGCCACGGCATCCTCGGCAACAGCCGATTCTGC
GCCCTCGTCAACGAGAAGTGCTCCGAACCCGCCAAGGAGCGCATGGCTCG
CATCCGCCGCTACGCCGCGGACGTGACGCGCTGCGGACCCCTCGCGCTCT
ACACCGCCATCGTCTCCAGCGCCAACCGTCTCATCCAGACCGACCCGTCG
TGCGACCTGGATCTCGCCGAATGTTACGTCGAGACGGCGTCCAAGAGGAA
CGCCGTCCCCCTCTCGGCCTTCTACCGCGACTGCGATCGCCTGCGGGATG
CTGTCGCCGCGTTCTTCAAGACCTACGGCATGGTGGTGGACGCCATGGCG
CAGCGCATCACGGAGCGGGTCGGGCCGGCCCTGGGTAGGGGCCTCTACTC
GACCGTCGTCATGATGGATCGCTGCGGAAACAGCTTCCAGGGACGCGAGG
AGACCCCCATCTCCGTCTTCGCCCGGGTCGCCGCGGCTCTCGCCGTCGAG
TGCGAGGTCGACGGGGGCGTCTCGTACAAGATCCTCAGCTCCAAGCCCGT
CGACGCCGCGCAGGCCTTCGACGCCTTCCTCTCCGCCCTCTGCTCCTTCG
CCATCATCCCCTCGCCGCGGGTCCTGGCCTACGCCGGGTTCGGCGGTTCC
AACCCGATCTTCGACGCCGTTTCTTACCGCGCTCAGTTCTACTCGGCCGA
GAGCACGATCAACGGCACCCTGCACGACATCTGCGACATGGTGACCAACG
GCCTCTCGGTGTCCGTCAGCGCGGCGGACCTCGGAGGCGACATCGTGGCC
TCTCTGCACATCCTCGGACAGCAGTGCAAGGCGCTGCGGCCGTACGCGCG
ATTCAAGACCGTCTTGAGGATCTACTTCGACATCTGGTCCGTCGACGCTC
TCAAGATCTTCTCTTTCATCCTCGACGTCGGGCGGGAATACGAGGGCCTG
ATGGCCTTCGCGGTCAACACGCCGAGGATCTTCTGGGATCGCTACCTAGA
CAGCTCCGGCGACAAGATGTGGCTCATGTTCGCGAGGCGGGAGGCCGCGG
CCCTGTGCGGCCTCGACCTCAAGTCCTTCCGTAACGTCTACGAGAAGATG
GAGCGTGACGGGCGCAGCGCCATCACCGTCTCGCCCTGGTGGGCCGTCTG
TCAGCTCGACGCGTGCGTGGCGCGGGGCAACACGGCCGTGGTCTTCCCTC
ACAACGTCAAGAGTATGATCCCCGAGAACATCGGGCGCCCCGCCGTGTGC
GGACCCGGCGTCTCCGTCGTCTCCGGCGGCTTCGTCGGCTGTACCCCCAT
CCACGAGCTGTGCATCAACCTGGAGAACTGCGTCCTGGAGGGCGCGGCAG
TCGAGAGCTCCGTCGACGTGGTCCTCGGTCTCGGTTGCCGCTTCAGCTTC
AAGGCCCTGGAGTCCCTGGTCCGCGACGCGGTGGTGCTGGGTAACCTGCT
CATCGACATGACCGTGCGCACCAACGCGTACGGCGCCGGCAAGCTCCTGA
CGCTCTATCGCGACCTGCACATCGGGGTCGTCGGCTTCCACGCTGTGATG
AATCGCCTCGGGCAGAAGTTCGCCGACATGGAGTCTTACGACCTCAACCA
GCGTATCGCGGAGTTTATCTACTACACCGCCGTGCGGGCCAGCGTCGACC
TGTGCATGGCGGGCGCCGATCCGTTCCCCAAGTTCCCTAAGAGCCTGTAC
GCGGCCGGCCGCTTCTACCCCGACCTCTTCGACGACGACGAGCGCGGCCC
GCGTCGCATGACCAAGGAGTTCCTCGAGAAACTACGTGAAGACGTGGTGA
AACACGGCATCAGGAACGCCTCCTTCATCACCGGCTGCTCGGCCGACGAA
GCCGCCAACCTGGCGGGCACCACTCCGGGCTTCTGGCCGCGCCGCGACAA
CGTCTTTCTCGAGCAGACGCCGCTCATGATGACCCCGACGAAGGATCAGA
TGCTCGATGAGTGCGTGCGCTCCGTCAAGATCGAGCCCCATCGCCTGCAC
GAGGAGGATCTCTCCTGTCTCGGCGAGAACCGGCCCGTCGAGCTGCCCGT
GCTCAACAGCCGCCTCAGGCAGATCTCGAAAGAGTCCGCGACGGTCGCCG
TGCGCCGCGGCCGCTCGGCGCCCTTCTACGACGACTCGGACGACGAGGAC
GAGGTGGCGTGCTCCGAGACCGGCTGGACCGTGTCGACCGACGCGGTCAT
CAAGATGTGCGTCGACAGACAGCCGTTCGTCGACCATGCGCAGTCGCTGC
CCGTCGCCATCGGCTTCGGGGGGTCTTCGGTGGAATTGGCGCGTCATCTG
AGACGAGGGAACGCTCTGGGACTGTCCGTCGGAGTATATAAATGTAGTAT
GCCCCCTTCCGTGAATTATCGC

Accordingly, preferably the Murine CTMV M45 polypeptide is encoded by the DNA nucleotide sequence substantially as set out in SEQ ID NO: 424, or a variant or fragment thereof.

One embodiment of codon optimised nucleic acid (DNA) encoding the Murine CTMV M45 polypeptide is provided herein as SEQ ID No: 425, as follows:

[SEQ ID No: 425]
ATGGACAGACAGCCCAAGGTGTACAGCGACCCCGACAACGGCTTCTTCTT
CCTGGATGTGCCCATGCCTGACGATGGCCAAGGCGGACAGCAGACAGCTA
CAACAGCTGCCGGCGGAGCCTTTGGAGTTGGCGGAGGACATTCTGTGCCC
TACGTGCGGATCATGAATGGCGTGTCCGGCATCCAGATCGGCAACCACAA
CGCCATGTCTATCGCCAGCTGTTGGAGCCCCAGCTACACCGATCGGCGGA
GAAGAAGCTACCCTAAGACCGCCACAAACGCCGCTGCCGATAGAGTGGCT
GCTGCTGTGTCTGCCGCTAACGCTGCTGTGAATGCTGCTGCCGCTGCTGC
AGCCGCAGGCGGCGGAGGCGGAGCTAATCTTCTTGCAGCAGCCGTGACCT
GCGCCAACCAGAGAGGATGTTGCGGAGGAAATGGCGGCCACAGCCTGCCT
CCAACCAGAATGCCTAAGACCAATGCCACAGCCGCTGCAGCTCCAGCAGT
TGCCGGTGCCTCTAATGCCAAGAGCGACAACAACCACGCCAACGCCACAT
CTGGCGCTGGATCTGCTGCTGCTACACCAGCCGCTACAACACCAGCTGCC
ACCGCCGTTGAGAACAGAAGGCCATCTCCAAGTCCTAGCACCGCCAGCAC
AGCCCCTTGTGATGAGGGAAGCAGCCCCAGACACCACAGACCTAGCCATG
TGTCTGTGGGCACACAGGCCACACCTAGCACACCAATTCCTATTCCAGCT
CCTCGGTGCTCCACAGGCCAGCAGCAACAACAGCCTCAGGCCAAGAAGCT
GAAGCCCGCCAAAGCTGACCCTCTGCTGTATGCCGCAACCATGCCTCCTC
CAGCCTCTGTGACTACTGCCGCAGCAGCTGCAGTGGCCCCTGAGTCTGAA
TCTTCTCCTGCCGCTTCTGCCCCTCCAGCAGCCGCCGCTATGGCTACAGG
CGGAGATGATGAGGACCAGAGCAGCTTCTCCTTCGTGTCCGATGATGTGC
TGGGCGAGTTCGAGGACCTGAGAATTGCTGGACTGCCCGTGCGGGATGAG
ATGAGGCCTCCTACACCTACCATGACAGTGATCCCCGTGTCTCGGCCTTT
CAGAGCCGGCAGAGATTCTGGCAGAGATGCCCTGTTCGACGACGCCGTGG
AAAGCGTGCGGTGTTACTGTCACGGCATCCTGGGCAACAGCAGATTCTGC
GCCCTGGTCAACGAGAAGTGTAGCGAGCCTGCCAAAGAACGGATGGCCCG
GATTAGAAGATACGCCGCCGACGTGACAAGATGCGGACCTCTGGCTCTGT
ACACCGCCATTGTGTCTAGCGCCAACCGGCTGATCCAGACAGACCCTAGC
TGTGACCTGGATCTGGCCGAGTGCTACGTGGAAACCGCCTCCAAGAGAAA
CGCCGTGCCTCTGAGCGCCTTCTACAGAGACTGCGACAGACTGAGAGATG
CCGTGGCCGCCTTCTTCAAGACCTACGGCATGGTGGTGGACGCCATGGCT
CAGAGAATCACCGAGAGAGTGGGACCCGCTCTCGGCAGAGGACTGTATTC
TACCGTGGTCATGATGGACAGATGCGGCAACAGCTTCCAGGGCAGAGAAG
AGACACCCATCAGCGTGTTCGCCAGAGTGGCTGCTGCTCTGGCCGTGGAA
TGTGAAGTGGATGGCGGCGTGTCCTACAAGATCCTGAGCAGCAAGCCTGT
GGATGCCGCTCAGGCCTTCGATGCCTTTCTGAGCGCCCTGTGCAGCTTCG
CCATCATTCCATCTCCAAGAGTGCTGGCCTACGCCGGCTTTGGCGGCAGC
AATCCTATCTTTGACGCCGTGTCTTACAGGGCCCAGTTCTACAGCGCCGA
GAGCACCATCAATGGCACCCTGCACGACATCTGCGACATGGTCACAAACG
GCCTGTCCGTGTCTGTGTCTGCCGCTGATCTCGGCGGAGATATCGTGGCC
TCTCTGCACATTCTGGGCCAGCAGTGCAAAGCCCTGAGGCCTTACGCCAG
ATTCAAGACCGTGCTGCGGATCTACTTCGACATTTGGAGCGTGGACGCCC
TGAAGATCTTTAGCTTCATCCTGGACGTGGGGCGCGAGTACGAAGGACTG
ATGGCCTTTGCCGTGAATACCCCTCGGATCTTCTGGGACAGATACCTGGA
CAGCAGCGGCGACAAGATGTGGCTGATGTTTGCCAGAAGAGAAGCCGCCG
CTCTGTGCGGCCTGGATCTGAAGTCCTTCCGGAACGTGTACGAGAAGATG
GAACGCGACGGCCGCTCTGCCATCACAGTTAGTCCTTGGTGGGCCGTGTG
TCAGCTGGATGCCTGTGTGGCCAGAGGCAATACCGCCGTGGTGTTCCCTC
ACAACGTGAAGTCTATGATCCCCGAGAACATCGGCAGGCCAGCCGTGTGT
GGACCTGGCGTTAGTGTTGTGTCTGGCGGCTTCGTGGGCTGCACACCTAT
TCACGAGCTGTGCATCAACCTGGAAAACTGCGTGCTGGAAGGCGCCGCTG
TGGAATCCTCTGTGGATGTGGTGCTCGGCCTGGGCTGCAGATTCAGCTTT
AAGGCCCTGGAAAGCCTCGTGCGGGATGCTGTGGTTCTGGGCAACCTGCT
GATCGACATGACCGTGCGGACCAATGCCTATGGCGCTGGCAAACTGCTGA
CCCTGTACCGGGATCTGCACATCGGCGTTGTGGGATTCCACGCCGTGATG
AACAGACTGGGCCAGAAATTCGCCGACATGGAAAGCTACGACCTGAACCA
GCGGATCGCCGAGTTCATCTACTACACAGCCGTCAGAGCCAGCGTGGACC
TGTGTATGGCTGGCGCCGATCCATTTCCTAAGTTCCCCAAGAGCCTGTAC
GCCGCTGGCAGATTCTACCCCGACCTGTTCGACGACGATGAGAGGGGCCC
TAGACGGATGACCAAAGAGTTCCTGGAAAAGCTGAGGGAAGATGTGGTCA
AGCACGGCATCCGGAACGCCAGCTTTATCACAGGCTGTAGCGCCGACGAG
GCCGCCAATCTTGCTGGAACAACACCCGGCTTTTGGCCCAGACGGGACAA
TGTGTTTCTGGAACAGACCCCTCTGATGATGACCCCTACCAAGGACCAGA
TGCTGGACGAGTGCGTGCGGAGCGTGAAGATCGAACCTCACAGACTGCAC
GAAGAGGACCTGAGCTGCCTGGGCGAGAACAGACCTGTGGAACTGCCCGT
GCTGAACAGCAGACTGCGGCAGATCAGCAAAGAAAGCGCCACCGTGGCCG
TGCGGAGAGGAAGAAGTGCTCCATTCTACGACGACAGCGACGACGAGGAT
GAAGTGGCCTGTTCTGAGACAGGCTGGACCGTGTCTACCGATGCCGTGAT
CAAGATGTGCGTGGACAGACAGCCCTTCGTGGATCACGCTCAGTCTCTGC
CTGTGGCCATCGGCTTTGGAGGCTCTAGCGTGGAACTGGCCAGACACCTG
AGAAGAGGCAATGCCCTGGGACTGTCTGTGGGCGTGTACAAGTGTAGCAT
GCCTCCTAGCGTGAACTACCGG

Hence, preferably the RNA construct is encoded by a DNA sequence substantially as set out in SEQ ID No: 425, or a fragment or variant thereof.

In an embodiment, the RNA sequence corresponding to the codon optimised DNA sequence of SEQ ID No: 425 is provided herein as SEQ ID No: 426, as follows:

[SEQ ID No: 426]
AUGGACAGACAGCCCAAGGUGUACAGCGACCCCGACAACGGCUUCUUCUU
CCUGGAUGUGCCCAUGCCUGACGAUGGCCAAGGCGGACAGCAGACAGCUA
CAACAGCUGCCGGCGGAGCCUUUGGAGUUGGCGGAGGACAUUCUGUGCCC
UACGUGCGGAUCAUGAAUGGCGUGUCCGGCAUCCAGAUCGGCAACCACAA
CGCCAUGUCUAUCGCCAGCUGUUGGAGCCCCAGCUACACCGAUCGGCGGA
GAAGAAGCUACCCUAAGACCGCCACAAACGCCGCUGCCGAUAGAGUGGCU
GCUGCUGUGUCUGCCGCUAACGCUGCUGUGAAUGCUGCUGCCGCUGCUGC
AGCCGCAGGCGGCGGAGGCGGAGCUAAUCUUCUUGCAGCAGCCGUGACCU
GCGCCAACCAGAGAGGAUGUUGCGGAGGAAAUGGCGGCCACAGCCUGCCU
CCAACCAGAAUGCCUAAGACCAAUGCCACAGCCGCUGCAGCUCCAGCAGU
UGCCGGUGCCUCUAAUGCCAAGAGCGACAACAACCACGCCAACGCCACAU
CUGGCGCUGGAUCUGCUGCUGCUACACCAGCCGCUACAACACCAGCUGCC
ACCGCCGUUGAGAACAGAAGGCCAUCUCCAAGUCCUAGCACCGCCAGCAC
AGCCCCUUGUGAUGAGGGAAGCAGCCCCAGACACCACAGACCUAGCCAUG
UGUCUGUGGGCACACAGGCCACACCUAGCACACCAAUUCCUAUUCCAGCU
CCUCGGUGCUCCACAGGCCAGCAGCAACAACAGCCUCAGGCCAAGAAGCU
GAAGCCCGCCAAAGCUGACCCUCUGCUGUAUGCCGCAACCAUGCCUCCUC
CAGCCUCUGUGACUACUGCCGCAGCAGCUGCAGUGGCCCCUGAGUCUGAA
UCUUCUCCUGCCGCUUCUGCCCCUCCAGCAGCCGCCGCUAUGGCUACAGG
CGGAGAUGAUGAGGACCAGAGCAGCUUCUCCUUCGUGUCCGAUGAUGUGC
UGGGCGAGUUCGAGGACCUGAGAAUUGCUGGACUGCCCGUGCGGGAUGAG
AUGAGGCCUCCUACACCUACCAUGACAGUGAUCCCCGUGUCUCGGCCUUU
CAGAGCCGGCAGAGAUUCUGGCAGAGAUGCCCUGUUCGACGACGCCGUGG
AAAGCGUGCGGUGUUACUGUCACGGCAUCCUGGGCAACAGCAGAUUCUGC
GCCCUGGUCAACGAGAAGUGUAGCGAGCCUGCCAAAGAACGGAUGGCCCG
GAUUAGAAGAUACGCCGCCGACGUGACAAGAUGCGGACCUCUGGCUCUGU
ACACCGCCAUUGUGUCUAGCGCCAACCGGCUGAUCCAGACAGACCCUAGC
UGUGACCUGGAUCUGGCCGAGUGCUACGUGGAAACCGCCUCCAAGAGAAA
CGCCGUGCCUCUGAGCGCCUUCUACAGAGACUGCGACAGACUGAGAGAUG
CCGUGGCCGCCUUCUUCAAGACCUACGGCAUGGUGGUGGACGCCAUGGCU
CAGAGAAUCACCGAGAGAGUGGGACCCGCUCUCGGCAGAGGACUGUAUUC
UACCGUGGUCAUGAUGGACAGAUGCGGCAACAGCUUCCAGGGCAGAGAAG
AGACACCCAUCAGCGUGUUCGCCAGAGUGGCUGCUGCUCUGGCCGUGGAA
UGUGAAGUGGAUGGCGGCGUGUCCUACAAGAUCCUGAGCAGCAAGCCUGU
GGAUGCCGCUCAGGCCUUCGAUGCCUUUCUGAGCGCCCUGUGCAGCUUCG
CCAUCAUUCCAUCUCCAAGAGUGCUGGCCUACGCCGGCUUUGGCGGCAGC
AAUCCUAUCUUUGACGCCGUGUCUUACAGGGCCCAGUUCUACAGCGCCGA
GAGCACCAUCAAUGGCACCCUGCACGACAUCUGCGACAUGGUCACAAACG
GCCUGUCCGUGUCUGUGUCUGCCGCUGAUCUCGGCGGAGAUAUCGUGGCC
UCUCUGCACAUUCUGGGCCAGCAGUGCAAAGCCCUGAGGCCUUACGCCAG
AUUCAAGACCGUGCUGCGGAUCUACUUCGACAUUUGGAGCGUGGACGCCC
UGAAGAUCUUUAGCUUCAUCCUGGACGUGGGGCGCGAGUACGAAGGACUG
AUGGCCUUUGCCGUGAAUACCCCUCGGAUCUUCUGGGACAGAUACCUGGA
CAGCAGCGGCGACAAGAUGUGGCUGAUGUUUGCCAGAAGAGAAGCCGCCG
CUCUGUGCGGCCUGGAUCUGAAGUCCUUCCGGAACGUGUACGAGAAGAUG
GAACGCGACGGCCGCUCUGCCAUCACAGUUAGUCCUUGGUGGGCCGUGUG
UCAGCUGGAUGCCUGUGUGGCCAGAGGCAAUACCGCCGUGGUGUUCCCUC
ACAACGUGAAGUCUAUGAUCCCCGAGAACAUCGGCAGGCCAGCCGUGUGU
GGACCUGGCGUUAGUGUUGUGUCUGGCGGCUUCGUGGGCUGCACACCUAU
UCACGAGCUGUGCAUCAACCUGGAAAACUGCGUGCUGGAAGGCGCCGCUG
UGGAAUCCUCUGUGGAUGUGGUGCUCGGCCUGGGCUGCAGAUUCAGCUUU
AAGGCCCUGGAAAGCCUCGUGCGGGAUGCUGUGGUUCUGGGCAACCUGCU
GAUCGACAUGACCGUGCGGACCAAUGCCUAUGGCGCUGGCAAACUGCUGA
CCCUGUACCGGGAUCUGCACAUCGGCGUUGUGGGAUUCCACGCCGUGAUG
AACAGACUGGGCCAGAAAUUCGCCGACAUGGAAAGCUACGACCUGAACCA
GCGGAUCGCCGAGUUCAUCUACUACACAGCCGUCAGAGCCAGCGUGGACC
UGUGUAUGGCUGGCGCCGAUCCAUUUCCUAAGUUCCCCAAGAGCCUGUAC
GCCGCUGGCAGAUUCUACCCCGACCUGUUCGACGACGAUGAGAGGGGCCC
UAGACGGAUGACCAAAGAGUUCCUGGAAAAGCUGAGGGAAGAUGUGGUCA
AGCACGGCAUCCGGAACGCCAGCUUUAUCACAGGCUGUAGCGCCGACGAG
GCCGCCAAUCUUGCUGGAACAACACCCGGCUUUUGGCCCAGACGGGACAA
UGUGUUUCUGGAACAGACCCCUCUGAUGAUGACCCCUACCAAGGACCAGA
UGCUGGACGAGUGCGUGCGGAGCGUGAAGAUCGAACCUCACAGACUGCAC
GAAGAGGACCUGAGCUGCCUGGGCGAGAACAGACCUGUGGAACUGCCCGU
GCUGAACAGCAGACUGCGGCAGAUCAGCAAAGAAAGCGCCACCGUGGCCG
UGCGGAGAGGAAGAAGUGCUCCAUUCUACGACGACAGCGACGACGAGGAU
GAAGUGGCCUGUUCUGAGACAGGCUGGACCGUGUCUACCGAUGCCGUGAU
CAAGAUGUGCGUGGACAGACAGCCCUUCGUGGAUCACGCUCAGUCUCUGC
CUGUGGCCAUCGGCUUUGGAGGCUCUAGCGUGGAACUGGCCAGACACCUG
AGAAGAGGCAAUGCCCUGGGACUGUCUGUGGGCGUGUACAAGUGUAGCAU
GCCUCCUAGCGUGAACUACCGG

In another embodiment, the inhibitor of an innate signalling pathway is Pangolin CoV-2 ORF3b*57 variant protein, (i.e. a mutated form the Pangolin CoV-2 ORF3b protein where a premature stop codon has been altered to AA glutamine (NCBI Reference Sequence: QIG55946.1; UniProtKB—A0A6M3G7Q4 (A0A6M3G7Q4_9BETC)), or an orthologue thereof (Konno Y, Kimura I, Uriu K, Fukushi M, Irie T, Koyanagi Y, Sauter D, Gifford R, USFQ-COVID19 Consortium, Nakagawa S and Sato K. 2020. SARS-CoV-2 ORF3b is a potent interferon antagonist whose activity is increased by a naturally occurring elongation variant. Cell Reports 32: Issue 12. doi.org/10.116/j.celrep.2020.108185. One embodiment of the Pangolin CoV-2 ORF3b*57 is represented herein as SEQ ID No: 427, as follows:

[SEQ ID No: 427]
MMLTTSCVGILIVTTIVFHTIVQLLQLSLPPVMAQQIPLQNMTTKLVVIL
RNGNLE

Therefore, preferably the RNA construct comprises a nucleotide sequence which encodes an amino acid sequence substantially as set out in SEQ ID No: 427, or a variant or fragment thereof.

In one embodiment, the Pangolin CoV-2 ORF3b*57 polypeptide is encoded by the DNA nucleotide sequence of SEQ ID No: 428, as follows:

[SEQ ID No: 428]
ATGATGCTAACTACTTCCTGTGTTGGCATACTAATTGTTACGACTATTGT
ATTCCATACAATAGTGCAACTTCTTCAATTGTCATTACCTCCGGTGATGG
CACAACAAATCCCATTACAGAACATGACTACCAAATTGGTGGTTATTTTG
AGAAATGGGAATCTGGAG

Accordingly, preferably the Pangolin CoV-2 ORF3b*57 polypeptide is encoded by the DNA nucleotide sequence substantially as set out in SEQ ID NO: 428, or a variant or fragment thereof.

Thus, the RNA construct may comprise an RNA nucleotide sequence of SEQ ID No: 429, as follows:

[SEQ ID No: 429]
AUGAUGCUAACUACUUCCUGUGUUGGCAUACUAAUUGUUACGACUAUUGU
AUUCCAUACAAUAGUGCAACUUCUUCAAUUGUCAUUACCUCCGGUGAUGG
CACAACAAAUCCCAUUACAGAACAUGACUACCAAAUUGGUGGUUAUUUUG
AGAAAUGGGAAUCUGGAG

Therefore, preferably the RNA construct comprises an RNA nucleotide sequence substantially as set out in SEQ ID No: 429, or a variant or fragment thereof.

The inventors then subjected the protein sequence of SEQ ID No: 442 to codon optimisation for human expression, and one embodiment of the codon optimised nucleic acid (DNA) sequence that includes that includes a start (ATG) and a stop (TGA) codon is provided herein as SEQ ID No: 430, as follows:

[SEQ ID No: 430]
ATGATGCTGACCACCAGCTGTGTGGGCATCCTGATCGTGACCACCATCGT
GTTCCACACAATCGTGCAGCTGCTGCAGCTCAGCCTGCCTCCTGTTATGG
CCCAGCAGATCCCTCTGCAGAACATGACCACAAAGCTGGTCGTGATCCTG
CGGAACGGCAACCTGGAATGA

Hence, preferably the RNA construct is encoded by a DNA sequence substantially as set out in SEQ ID No: 430, or a fragment or variant thereof.

In an embodiment, the RNA sequence corresponding to the codon optimised DNA sequence of SEQ ID No: 430 that includes a start (AUG) and a stop (UGA) codon is provided herein as SEQ ID No: 431, as follows:

[SEQ ID No: 431]
AUGAUGCUGACCACCAGCUGUGUGGGCAUCCUGAUCGUGACCACCAUCGU
GUUCCACACAAUCGUGCAGCUGCUGCAGCUCAGCCUGCCUCCUGUUAUGG
CCCAGCAGAUCCCUCUGCAGAACAUGACCACAAAGCUGGUCGUGAUCCUG
CGGAACGGCAACCUGGAAUGA

Hence, preferably the RNA construct comprises a sequence substantially as set out in SEQ ID No: 431, or a fragment or variant thereof.

In another embodiment, the inhibitor of an innate signalling pathway is Pangolin CoV-2 ORF3b*79 variant protein, (i.e. a mutated form the Pangolin CoV-2 ORF3b protein where a premature stop codon has been altered to AA glutamine (NCBI Reference Sequence: QIG55946.1; UniProtKB—A0A6M3G7Q4 (A0A6M3G7Q4_9BETC)), or an orthologue thereof (Konno Y, Kimura I, Uriu K, Fukushi M, Irie T, Koyanagi Y, Sauter D, Gifford R, USFQ-COVID19 Consortium, Nakagawa S and Sato K. 2020. SARS-CoV-2 ORF3b is a potent interferon antagonist whose activity is increased by a naturally occurring elongation variant. Cell Reports 32: Issue 12. doi.org/10.116/j.celrep.2020.108185. One embodiment of the Pangolin CoV-2 ORF3b*79 is represented herein as SEQ ID No: 432, as follows:

[SEQ ID No: 432]
MMLTTSCVGILIVTTIVFHTIVQLLQLSLPPVMAQQIPLQNMTTKLVVIL
RNGNLELKTVLYYTATSLQITTSCTQLN

Therefore, preferably the RNA construct comprises a nucleotide sequence which encodes an amino acid sequence substantially as set out in SEQ ID No: 432, or a variant or fragment thereof.

In one embodiment, the Pangolin CoV-2 ORF3b*79 polypeptide is encoded by the DNA nucleotide sequence of SEQ ID No: 433, as follows:

[SEQ ID No: 433]
ATGATGCTAACTACTTCCTGTGTTGGCATACTAATTGTTACGACTATTGT
ATTCCATACAATAGTGCAACTTCTTCAATTGTCATTACCTCCGGTGATGG
CACAACAAATCCCATTACAGAACATGACTACCAAATTGGTGGTTATTTTG
AGAAATGGGAATCTGGAGCTGAAGACTGTGTTGTATTACACAGCTACTTC
ACTTCAGATTACTACCAGCTGTACTCAACTCAAT

Accordingly, preferably the Pangolin CoV-2 ORF3b*79 polypeptide is encoded by the DNA nucleotide sequence substantially as set out in SEQ ID NO: 433, or a variant or fragment thereof.

Thus, the RNA construct may comprise an RNA nucleotide sequence of SEQ ID No: 434, as follows:

[SEQ ID No: 434]
AUGAUGCUAACUACUUCCUGUGUUGGCAUACUAAUUGUUACGACUAUUGU
AUUCCAUACAAUAGUGCAACUUCUUCAAUUGUCAUUACCUCCGGUGAUGG
CACAACAAAUCCCAUUACAGAACAUGACUACCAAAUUGGUGGUUAUUUUG
AGAAAUGGGAAUCUGGAGCUGAAGACUGUGUUGUAUUACACAGCUACUUC
ACUUCAGAUUACUACCAGCUGUACUCAACUCAAU

Therefore, preferably the RNA construct comprises an RNA nucleotide sequence substantially as set out in SEQ ID No: 434, or a variant or fragment thereof.

The inventors then subjected the protein sequence of SEQ ID No: 432 to codon optimisation for human expression, and one embodiment of the codon optimised nucleic acid (DNA) sequence that includes that includes a start (ATG) and a stop (TGA) codon is provided herein as SEQ ID No: 435, as follows:

[SEQ ID No: 435]
ATGATGCTGACCACCAGCTGTGTGGGCATCCTGATCGTGACCACCATCGTG
TTCCACACAATCGTGCAGCTGCTGCAGCTCAGCCTGCCTCCTGTTATGGCC
CAGCAGATCCCTCTGCAGAACATGACCACAAAGCTGGTCGTGATCCTGCGG
AACGGCAACCTGGAACTGAAAACCGTGCTGTACTACACCGCCACCAGCCTG
CAGATCACCACAAGCTGCACCCAGCTGAACTGA

Hence, preferably the RNA construct is encoded by a DNA sequence substantially as set out in SEQ ID No: 435, or a fragment or variant thereof.

In an embodiment, the RNA sequence corresponding to the codon optimised DNA sequence of SEQ ID No: 435 that includes a start (AUG) and a stop (UGA) codon is provided herein as SEQ ID No: 436, as follows:

[SEQ ID No: 436]
AUGAUGCUGACCACCAGCUGUGUGGGCAUCCUGAUCGUGACCACCAUCGUG
UUCCACACAAUCGUGCAGCUGCUGCAGCUCAGCCUGCCUCCUGUUAUGGCC
CAGCAGAUCCCUCUGCAGAACAUGACCACAAAGCUGGUCGUGAUCCUGCGG
AACGGCAACCUGGAACUGAAAACCGUGCUGUACUACACCGCCACCAGCCUG
CAGAUCACCACAAGCUGCACCCAGCUGAACUGA

Hence, preferably the RNA construct comprises a sequence substantially as set out in SEQ ID No: 436, or a fragment or variant thereof.

In another embodiment, the inhibitor of an innate signalling pathway is MERS-ORF4a protein (NCBI Reference Sequence: AGV08457.1; UniProtKB—T2BBG6 (T2BBG6_MERS)), or an orthologue thereof. One embodiment of the MERS-ORF4a is represented herein as SEQ ID No: 437, as follows:

[SEQ ID No: 437]
MDYVSLLNQIWQKYLNSPYTTCLYIPKPTAKYTPLVGTSLHPVLWNCQLSF
AGYTESAVNSTKALAKQDAAQRIAWLLHKDGGIPDGCSLYLRHSSLFAQSE
EEESFSN

Therefore, preferably the RNA construct comprises a nucleotide sequence which encodes an amino acid sequence substantially as set out in SEQ ID No: 437, or a variant or fragment thereof.

In one embodiment, the MERS-ORF4a polypeptide is encoded by the DNA nucleotide sequence of SEQ ID No: 438, as follows:

[SEQ ID No: 438]
ATGGACTACGTGTCCCTGCTGAACCAGATTTGGCAGAAGTACCTGAACAGC
CCCTACACCACCTGTCTGTACATCCCCAAGCCTACCGCCAAGTACACACCT
CTCGTGGGCACATCTCTGCACCCCGTGCTGTGGAATTGCCAGCTGAGCTTT
GCCGGCTACACCGAGTCTGCCGTGAACAGCACAAAGGCCCTGGCCAAACAG
GACGCCGCTCAGAGAATTGCCTGGCTGCTGCACAAGGATGGCGGCATCCCT
GATGGCTGTAGCCTGTACCTGAGACACAGCAGCCTGTTCGCCCAGAGCGAG
GAAGAGGAATCCTTCAGCAAC

Accordingly, preferably the MERS-ORF4a polypeptide is encoded by the DNA nucleotide sequence substantially as set out in SEQ ID NO: 438, or a variant or fragment thereof.

Thus, the RNA construct may comprise an RNA nucleotide sequence of SEQ ID No: 439, as follows:

[SEQ ID No: 439]
AUGGACUACGUGUCCCUGCUGAACCAGAUUUGGCAGAAGUACCUGAACAGC
CCCUACACCACCUGUCUGUACAUCCCCAAGCCUACCGCCAAGUACACACCU
CUCGUGGGCACAUCUCUGCACCCCGUGCUGUGGAAUUGCCAGCUGAGCUUU
GCCGGCUACACCGAGUCUGCCGUGAACAGCACAAAGGCCCUGGCCAAACAG
GACGCCGCUCAGAGAAUUGCCUGGCUGCUGCACAAGGAUGGCGGCAUCCCU
GAUGGCUGUAGCCUGUACCUGAGACACAGCAGCCUGUUCGCCCAGAGCGAG
GAAGAGGAAUCCUUCAGCAAC

Therefore, preferably the RNA construct comprises an RNA nucleotide sequence substantially as set out in SEQ ID No: 439, or a variant or fragment thereof.

The inventors then subjected the protein sequence of SEQ ID No: 437 to codon optimisation for human expression, and one embodiment of the codon optimised nucleic acid (DNA) sequence that includes that includes a start (ATG) and a stop (TGA) codon is provided herein as SEQ ID No: 440, as follows:

[SEQ ID No: 440]
ATGGACTACGTGTCCCTGCTGAATCAGATCTGGCAGAAGTACCTGAACAGC
CCCTACACCACCTGTCTGTACATCCCCAAGCCTACCGCCAAGTACACACCT
CTCGTGGGCACATCTCTGCACCCCGTGCTGTGGAATTGCCAGCTGAGCTTT
GCCGGCTACACCGAGAGCGCCGTGAATAGCACAAAGGCCCTGGCCAAACAG
GACGCCGCTCAGAGAATTGCCTGGCTGCTGCACAAGGATGGCGGCATCCCT
GATGGCTGTAGCCTGTACCTGAGACACAGCAGCCTGTTCGCCCAGAGCGAG
GAAGAGGAATCCTTCAGCAACTGA

Hence, preferably the RNA construct is encoded by a DNA sequence substantially as set out in SEQ ID No: 440 or a fragment or variant thereof.

In an embodiment, the RNA sequence corresponding to the codon optimised DNA sequence of SEQ ID No: 440 that includes a start (AUG) and a stop (UGA) codon is provided herein as SEQ ID No: 441, as follows:

[SEQ ID No: 441]
AUGGACUACGUGUCCCUGCUGAAUCAGAUCUGGCAGAAGUACCUGAACAGC
CCCUACACCACCUGUCUGUACAUCCCCAAGCCUACCGCCAAGUACACACCU
CUCGUGGGCACAUCUCUGCACCCCGUGCUGUGGAAUUGCCAGCUGAGCUUU
GCCGGCUACACCGAGAGCGCCGUGAAUAGCACAAAGGCCCUGGCCAAACAG
GACGCCGCUCAGAGAAUUGCCUGGCUGCUGCACAAGGAUGGCGGCAUCCCU
GAUGGCUGUAGCCUGUACCUGAGACACAGCAGCCUGUUCGCCCAGAGCGAG
GAAGAGGAAUCCUUCAGCAACUGA

Hence, preferably the RNA construct comprises a sequence substantially as set out in SEQ ID No: 441, or a fragment or variant thereof.

In another embodiment, the inhibitor of an innate signalling pathway is BVDV nPro protein (NCBI Reference Sequence: AAA42854.1; UniProtKB—P19711 (POLG_BVDVN)), or an orthologue thereof. One embodiment of the BVDV nPro is represented herein as SEQ ID No:442, as follows:

[SEQ ID No: 442]
MELITNELLYKTYKQKPVGVEEPVYDQAGDPLFGERGAVHPQSTLKLPHKR
GERDVPTNLASLPKRGDCRTGNSRGPVSGIYLKPGPLFYQDYKGPVYHRAP
LELFEEGSMCETTKRIGRVTGSDGKLYHIYVCIDGCIIIKSATRSYQRVFR
WVHNRLDCPLWVTSC

Therefore, preferably the RNA construct comprises a nucleotide sequence which encodes an amino acid sequence substantially as set out in SEQ ID No: 442, or a variant or fragment thereof.

In one embodiment, the BVDV nPro polypeptide is encoded by the DNA nucleotide sequence of SEQ ID No: 443, as follows:

[SEQ ID No: 443]
ATGGAGTTGATCACAAATGAACTTTTATACAAAACATACAAACAAAAACCC
GTCGGGGTGGAGGAACCTGTTTATGATCAGGCAGGTGATCCCTTATTTGGT
GAAAGGGGAGCAGTCCACCCTCAATCGACGCTAAAGCTCCCACACAAGAGA
GGGGAACGCGATGTTCCAACCAACTTGGCATCCTTACCAAAAAGAGGTGAC
TGCAGGACGGGTAATAGCAGAGGACCTGTGAGCGGGATCTACCTGAAGCCA
GGGCCACTATTTTACCAGGACTATAAAGGTCCCGTCTATCACAGGGCCCCG
CTGGAGCTCTTTGAGGAGGGATCCATGTGTGAAACGACTAAACGGATAGGG
AGAGTAACTGGAAGTGACGGAAAGCTGTACCACATTTATGTGTGTATAGAT
GGATGTATAATAATAAAAAGTGCCACGAGAAGTTACCAAAGGGTGTTCAGG
TGGGTCCATAATAGGCTTGACTGCCCTCTATGGGTCACAAGTTGC

Accordingly, preferably the BVDV nPro polypeptide is encoded by the DNA nucleotide sequence substantially as set out in SEQ ID NO:443, or a variant or fragment thereof.

Thus, the RNA construct may comprise an RNA nucleotide sequence of SEQ ID No:444, as follows:

[SEQ ID No: 444]
AUGGAGUUGAUCACAAAUGAACUUUUAUACAAAACAUACAAACAAAAACCC
GUCGGGGUGGAGGAACCUGUUUAUGAUCAGGCAGGUGAUCCCUUAUUUGGU
GAAAGGGGAGCAGUCCACCCUCAAUCGACGCUAAAGCUCCCACACAAGAGA
GGGGAACGCGAUGUUCCAACCAACUUGGCAUCCUUACCAAAAAGAGGUGAC
UGCAGGACGGGUAAUAGCAGAGGACCUGUGAGCGGGAUCUACCUGAAGCCA
GGGCCACUAUUUUACCAGGACUAUAAAGGUCCCGUCUAUCACAGGGCCCCG
CUGGAGCUCUUUGAGGAGGGAUCCAUGUGUGAAACGACUAAACGGAUAGGG
AGAGUAACUGGAAGUGACGGAAAGCUGUACCACAUUUAUGUGUGUAUAGAU
GGAUGUAUAAUAAUAAAAAGUGCCACGAGAAGUUACCAAAGGGUGUUCAGG
UGGGUCCAUAAUAGGCUUGACUGCCCUCUAUGGGUCACAAGUUGC

Therefore, preferably the RNA construct comprises an RNA nucleotide sequence substantially as set out in SEQ ID No:444, or a variant or fragment thereof.

The inventors then subjected the protein sequence of SEQ ID No: 442 to codon optimisation for human expression, and one embodiment of the codon optimised nucleic acid (DNA) sequence that includes that includes a start (ATG) and a stop (TGA) codon is provided herein as SEQ ID No: 445, as follows:

[SEQ ID No: 445]
ATGGAACTGATCACCAACGAGCTGCTGTACAAGACCTACAAGCAGAAACCC
GTGGGCGTCGAGGAACCCGTGTATGATCAAGCTGGCGACCCTCTGTTTGGC
GAGAGAGGCGCTGTTCACCCTCAGAGCACACTGAAGCTGCCCCACAAGCGG
GGCGAAAGAGATGTGCCTACCAACCTGGCCAGCCTGCCTAAGAGAGGCGAT
TGCAGAACCGGCAATAGCAGAGGCCCTGTGTCCGGCATCTACCTGAAACCT
GGACCACTGTTCTACCAGGACTACAAGGGCCCCGTGTACCACAGAGCACCC
CTGGAACTTTTCGAAGAGGGCAGCATGTGCGAAACCACCAAGCGGATCGGA
AGAGTGACCGGCTCTGACGGCAAGCTGTACCACATCTACGTGTGCATCGAC
GGCTGCATCATCATCAAGAGCGCCACCAGATCCTACCAGCGGGTGTTCAGA
TGGGTGCACAACAGACTGGACTGCCCTCTGTGGGTCACCAGCTGCTGA

Hence, preferably the RNA construct is encoded by a DNA sequence substantially as set out in SEQ ID No: 445, or a fragment or variant thereof.

In an embodiment, the RNA sequence corresponding to the codon optimised DNA sequence of SEQ ID No: 445 that includes a start (AUG) and a stop (UGA) codon is provided herein as SEQ ID No: 446, as follows:

[SEQ ID No: 446]
AUGGAACUGAUCACCAACGAGCUGCUGUACAAGACCUACAAGCAGAAACCC
GUGGGCGUCGAGGAACCCGUGUAUGAUCAAGCUGGCGACCCUCUGUUUGGC
GAGAGAGGCGCUGUUCACCCUCAGAGCACACUGAAGCUGCCCCACAAGCGG
GGCGAAAGAGAUGUGCCUACCAACCUGGCCAGCCUGCCUAAGAGAGGCGAU
UGCAGAACCGGCAAUAGCAGAGGCCCUGUGUCCGGCAUCUACCUGAAACCU
GGACCACUGUUCUACCAGGACUACAAGGGCCCCGUGUACCACAGAGCACCC
CUGGAACUUUUCGAAGAGGGCAGCAUGUGCGAAACCACCAAGCGGAUCGGA
AGAGUGACCGGCUCUGACGGCAAGCUGUACCACAUCUACGUGUGCAUCGAC
GGCUGCAUCAUCAUCAAGAGCGCCACCAGAUCCUACCAGCGGGUGUUCAGA
UGGGUGCACAACAGACUGGACUGCCCUCUGUGGGUCACCAGCUGCUGA

Hence, preferably the RNA construct comprises a sequence substantially as set out in SEQ ID No:446, or a fragment or variant thereof.

In another embodiment, the inhibitor of an innate signalling pathway is HSV US1 protein (NCBI Reference Sequence: CAB06708.1; UniProtKB—P89474 (ICP22_HHV2H)), or an orthologue thereof. One embodiment of the HSV US1 is represented herein as SEQ ID No: 447, as follows:

[SEQ ID No: 447]
MVRDCYLMGYCRTRLGPRTWGRLLQISGGTWDVRLRNAIREVEAHFEPAAE
PVCELPCLNARRYGPECDVGNLETNGGSTSDDEISDATDSDDTLASHSDTE
GGPSPAGRENPESASGGAIAARLECEFGTFDWTSEEGSQPWLSAVVADTSS
AERSGLPAPGACRATEAPEREDGCRKMRFPAACPYPCGHTFLRP

Therefore, preferably the RNA construct comprises a nucleotide sequence which encodes an amino acid sequence substantially as set out in SEQ ID No: 447, or a variant or fragment thereof.

In one embodiment, the HSV US1 polypeptide is encoded by the DNA nucleotide sequence of SEQ ID No:448, as follows:

[SEQ ID No: 448]
ATGGTGCGAGACTGCTACCTCATGGGCTACTGCCGGACCCGCCTGGGGCCG
CGCACGTGGGGCCGCCTGCTGCAGATCTCGGGCGGAACCTGGGACGTGCGC
CTGCGAAACGCAATCCGGGAGGTCGAGGCGCATTTTGAACCCGCCGCCGAG
CCCGTGTGCGAGCTGCCCTGTCTGAACGCCAGGCGTTACGGCCCCGAGTGT
GATGTTGGCAATCTCGAGACCAACGGCGGCTCGACGAGCGATGATGAGATA
TCGGATGCGACGGACTCGGACGATACCCTCGCGTCCCATTCCGACACGGAG
GGGGGGCCCTCCCCGGCCGGCCGGGAGAACCCGGAATCCGCGTCCGGCGGG
GCTATCGCGGCTCGGCTGGAGTGTGAGTTTGGGACGTTTGACTGGACGTCC
GAGGAGGGCTCCCAGCCCTGGCTGTCCGCGGTGGTCGCCGATACCAGCTCC
GCCGAACGCTCTGGCCTACCCGCCCCGGGCGCGTGTCGCGCAACGGAAGCC
CCAGAACGCGAGGACGGGTGCCGAAAAATGCGCTTCCCCGCCGCCTGCCCC
TATCCCTGCGGCCACACATTTCTCCGGCCA

Accordingly, preferably the HSV US1 polypeptide is encoded by the DNA nucleotide sequence substantially as set out in SEQ ID NO:448, or a variant or fragment thereof.

Thus, the RNA construct may comprise an RNA nucleotide sequence of SEQ ID No: 449, as follows:

[SEQ ID No: 449]
AUGGUGCGAGACUGCUACCUCAUGGGCUACUGCCGGACCCGCCUGGGGCCG
CGCACGUGGGGCCGCCUGCUGCAGAUCUCGGGCGGAACCUGGGACGUGCGC
CUGCGAAACGCAAUCCGGGAGGUCGAGGCGCAUUUUGAACCCGCCGCCGAG
CCCGUGUGCGAGCUGCCCUGUCUGAACGCCAGGCGUUACGGCCCCGAGUGU
GAUGUUGGCAAUCUCGAGACCAACGGCGGCUCGACGAGCGAUGAUGAGAUA
UCGGAUGCGACGGACUCGGACGAUACCCUCGCGUCCCAUUCCGACACGGAG
GGGGGGCCCUCCCCGGCCGGCCGGGAGAACCCGGAAUCCGCGUCCGGCGGG
GCUAUCGCGGCUCGGCUGGAGUGUGAGUUUGGGACGUUUGACUGGACGUCC
GAGGAGGGCUCCCAGCCCUGGCUGUCCGCGGUGGUCGCCGAUACCAGCUCC
GCCGAACGCUCUGGCCUACCCGCCCCGGGCGCGUGUCGCGCAACGGAAGCC
CCAGAACGCGAGGACGGGUGCCGAAAAAUGCGCUUCCCCGCCGCCUGCCCC
UAUCCCUGCGGCCACACAUUUCUCCGGCCA

Therefore, preferably the RNA construct comprises an RNA nucleotide sequence substantially as set out in SEQ ID No: 449, or a variant or fragment thereof.

The inventors then subjected the protein sequence of SEQ ID No: 447 to codon optimisation for human expression, and one embodiment of the codon optimised nucleic acid (DNA) sequence that includes that includes a start (ATG) and a stop (TGA) codon is provided herein as SEQ ID No:450, as follows:

[SEQ ID No: 450]
ATGGTCCGAGACTGCTACCTGATGGGCTACTGCAGAACCAGACTGGGCCC
TAGAACATGGGGCAGACTGCTGCAGATCTCTGGCGGCACATGGGATGTGC
GGCTGAGAAACGCCATCAGAGAGGTGGAAGCCCACTTCGAGCCTGCCGCT
GAACCTGTGTGTGAACTGCCCTGTCTGAACGCTAGAAGATACGGCCCTGA
GTGCGACGTGGGCAACCTGGAAACAAATGGCGGCAGCACCAGCGACGACG
AGATCTCTGATGCCACCGACAGCGACGATACACTGGCCAGCCACAGCGAT
ACAGAAGGCGGACCATCTCCTGCCGGAAGAGAGAATCCTGAGTCTGCCTC
TGGCGGAGCTATCGCCGCTAGACTGGAATGCGAGTTCGGCACCTTCGACT
GGACAAGCGAGGAAGGCTCTCAGCCTTGGCTGTCTGCTGTGGTGGCCGAT
ACAAGCAGCGCCGAAAGATCTGGACTTCCTGCTCCTGGCGCCTGCAGAGC
TACAGAAGCTCCTGAAAGAGAGGACGGCTGCAGAAAGATGCGGTTCCCTG
CCGCCTGTCCTTATCCTTGCGGCCACACATTTCTGCGGCCCTGA

Hence, preferably the RNA construct is encoded by a DNA sequence substantially as set out in SEQ ID No: 450, or a fragment or variant thereof.

In an embodiment, the RNA sequence corresponding to the codon optimised DNA sequence of SEQ ID No: 450 that includes a start (AUG) and a stop (UGA) codon is provided herein as SEQ ID No:451, as follows:

[SEQ ID No: 451]
AUGGUCCGAGACUGCUACCUGAUGGGCUACUGCAGAACCAGACUGGGCCC
UAGAACAUGGGGCAGACUGCUGCAGAUCUCUGGCGGCACAUGGGAUGUGC
GGCUGAGAAACGCCAUCAGAGAGGUGGAAGCCCACUUCGAGCCUGCCGCU
GAACCUGUGUGUGAACUGCCCUGUCUGAACGCUAGAAGAUACGGCCCUGA
GUGCGACGUGGGCAACCUGGAAACAAAUGGCGGCAGCACCAGCGACGACG
AGAUCUCUGAUGCCACCGACAGCGACGAUACACUGGCCAGCCACAGCGAU
ACAGAAGGCGGACCAUCUCCUGCCGGAAGAGAGAAUCCUGAGUCUGCCUC
UGGCGGAGCUAUCGCCGCUAGACUGGAAUGCGAGUUCGGCACCUUCGACU
GGACAAGCGAGGAAGGCUCUCAGCCUUGGCUGUCUGCUGUGGUGGCCGAU
ACAAGCAGCGCCGAAAGAUCUGGACUUCCUGCUCCUGGCGCCUGCAGAGC
UACAGAAGCUCCUGAAAGAGAGGACGGCUGCAGAAAGAUGCGGUUCCCUG
CCGCCUGUCCUUAUCCUUGCGGCCACACAUUUCUGCGGCCCUGA

Hence, preferably the RNA construct comprises a sequence substantially as set out in SEQ ID No: 452 or a fragment or variant thereof.

In another embodiment, the inhibitor of an innate signalling pathway is MERS CoV M protein (NCBI Reference Sequence: AGV08396.1; UniProtKB—T2BB40 (T2BB40_MERS)), or an orthologue thereof. One embodiment of the MERS CoV M is represented herein as SEQ ID No: 452, as follows:

[SEQ ID No: 452]
MSNMTQLTEAQIIAIIKDWNFAWSLIFLLITIVLQYGYPSRSMTVYVFKM
FVLWLLWPSSMALSIFSAIYPIDLASQIISGIVAAVSAMMWISYFVQSIR
LFMRTGSWWSFNPETNCLLNVPFGGTTVVRPLVEDSTSVTAVVTNGHLKM
AGMHFGACDYDRLPNEVTVAKPNVLIALKMVKRQSYGTNSGVAIYHRYKA
GNYRSPPITADIELALLR

Therefore, preferably the RNA construct comprises a nucleotide sequence which encodes an amino acid sequence substantially as set out in SEQ ID No: 452, or a variant or fragment thereof.

In one embodiment, the MERS CoV M polypeptide is encoded by the DNA nucleotide sequence of SEQ ID No: 453, as follows:

[SEQ ID No: 453]
ATGTCTAATATGACGCAACTCACTGAGGCGCAGATTATTGCCATTATTAA
AGACTGGAACTTTGCATGGTCCCTGATCTTTCTCTTAATTACTATCGTAC
TACAGTATGGATACCCATCCCGTAGTATGACTGTCTATGTCTTTAAAATG
TTTGTTTTATGGCTCCTATGGCCATCTTCCATGGCGCTATCAATATTTAG
CGCCATTTATCCAATTGATCTAGCTTCCCAGATAATCTCTGGCATTGTAG
CAGCTGTTTCAGCTATGATGTGGATTTCCTACTTTGTGCAGAGTATCCGG
CTGTTTATGAGAACTGGATCATGGTGGTCATTCAATCCTGAGACTAATTG
CCTTTTGAACGTTCCATTTGGTGGTACAACTGTCGTACGTCCACTCGTAG
AGGACTCCACCAGTGTAACTGCTGTTGTAACCAATGGCCACCTCAAAATG
GCTGGCATGCATTTCGGTGCTTGTGACTACGACAGACTTCCTAATGAAGT
CACCGTGGCCAAACCCAATGTGCTGATTGCTTTAAAAATGGTGAAGCGGC
AAAGCTACGGAACTAATTCCGGCGTTGCCATTTACCATAGATATAAGGCA
GGTAATTACAGGAGTCCGCCTATTACGGCGGATATTGAACTTGCATTGCT
TCGA

Accordingly, preferably the MERS CoV M polypeptide is encoded by the DNA nucleotide sequence substantially as set out in SEQ ID NO: 453, or a variant or fragment thereof.

Thus, the RNA construct may comprise an RNA nucleotide sequence of SEQ ID No: 454, as follows:

[SEQ ID No: 454]
AUGUCUAAUAUGACGCAACUCACUGAGGCGCAGAUUAUUGCCAUUAUUAA
AGACUGGAACUUUGCAUGGUCCCUGAUCUUUCUCUUAAUUACUAUCGUAC
UACAGUAUGGAUACCCAUCCCGUAGUAUGACUGUCUAUGUCUUUAAAAUG
UUUGUUUUAUGGCUCCUAUGGCCAUCUUCCAUGGCGCUAUCAAUAUUUAG
CGCCAUUUAUCCAAUUGAUCUAGCUUCCCAGAUAAUCUCUGGCAUUGUAG
CAGCUGUUUCAGCUAUGAUGUGGAUUUCCUACUUUGUGCAGAGUAUCCGG
CUGUUUAUGAGAACUGGAUCAUGGUGGUCAUUCAAUCCUGAGACUAAUUG
CCUUUUGAACGUUCCAUUUGGUGGUACAACUGUCGUACGUCCACUCGUAG
AGGACUCCACCAGUGUAACUGCUGUUGUAACCAAUGGCCACCUCAAAAUG
GCUGGCAUGCAUUUCGGUGCUUGUGACUACGACAGACUUCCUAAUGAAGU
CACCGUGGCCAAACCCAAUGUGCUGAUUGCUUUAAAAAUGGUGAAGCGGC
AAAGCUACGGAACUAAUUCCGGCGUUGCCAUUUACCAUAGAUAUAAGGCA
GGUAAUUACAGGAGUCCGCCUAUUACGGCGGAUAUUGAACUUGCAUUGCU
UCGA

Therefore, preferably the RNA construct comprises an RNA nucleotide sequence substantially as set out in SEQ ID No: 454, or a variant or fragment thereof.

The inventors then subjected the protein sequence of SEQ ID No: 452 to codon optimisation for human expression, and one embodiment of the codon optimised nucleic acid (DNA) sequence that includes that includes a start (ATG) and a stop (TGA) codon is provided herein as SEQ ID No:455, as follows:

[SEQ ID No: 455]
ATGAGCAACATGACCCAGCTGACAGAGGCCCAGATCATTGCCATCATCAA
GGACTGGAACTTCGCTTGGAGCCTGATCTTCCTGCTGATCACCATCGTGC
TGCAGTACGGCTACCCCAGCAGATCCATGACCGTGTACGTGTTCAAGATG
TTCGTCCTGTGGCTGCTGTGGCCCAGCTCTATGGCCCTGAGCATCTTCAG
CGCCATCTATCCCATCGACCTGGCCAGCCAGATCATCTCTGGAATCGTGG
CCGCCGTGTCCGCCATGATGTGGATCAGCTACTTCGTGCAGAGCATCCGG
CTGTTCATGAGAACCGGCAGCTGGTGGTCCTTCAATCCCGAGACAAACTG
CCTGCTGAACGTGCCCTTTGGCGGCACTACAGTCGTCAGACCCCTGGTGG
AAGATAGCACCTCTGTGACCGCCGTGGTCACCAATGGCCACCTGAAAATG
GCCGGCATGCACTTCGGCGCCTGCGACTATGACAGACTGCCCAACGAAGT
GACCGTGGCCAAGCCTAATGTGCTGATCGCCCTGAAGATGGTCAAGCGGC
AGAGCTACGGCACCAATTCTGGCGTGGCCATCTACCACAGATACAAGGCC
GGCAACTACAGAAGCCCTCCTATCACCGCCGACATCGAGCTGGCTCTGCT
GAGATGA

Hence, preferably the RNA construct is encoded by a DNA sequence substantially as set out in SEQ ID No: 455, or a fragment or variant thereof.

In an embodiment, the RNA sequence corresponding to the codon optimised DNA sequence of SEQ ID No: 455 that includes a start (AUG) and a stop (UGA) codon is provided herein as SEQ ID No: 456, as follows:

[SEQ ID No: 456]
AUGAGCAACAUGACCCAGCUGACAGAGGCCCAGAUCAUUGCCAUCAUCAA
GGACUGGAACUUCGCUUGGAGCCUGAUCUUCCUGCUGAUCACCAUCGUGC
UGCAGUACGGCUACCCCAGCAGAUCCAUGACCGUGUACGUGUUCAAGAUG
UUCGUCCUGUGGCUGCUGUGGCCCAGCUCUAUGGCCCUGAGCAUCUUCAG
CGCCAUCUAUCCCAUCGACCUGGCCAGCCAGAUCAUCUCUGGAAUCGUGG
CCGCCGUGUCCGCCAUGAUGUGGAUCAGCUACUUCGUGCAGAGCAUCCGG
CUGUUCAUGAGAACCGGCAGCUGGUGGUCCUUCAAUCCCGAGACAAACUG
CCUGCUGAACGUGCCCUUUGGCGGCACUACAGUCGUCAGACCCCUGGUGG
AAGAUAGCACCUCUGUGACCGCCGUGGUCACCAAUGGCCACCUGAAAAUG
GCCGGCAUGCACUUCGGCGCCUGCGACUAUGACAGACUGCCCAACGAAGU
GACCGUGGCCAAGCCUAAUGUGCUGAUCGCCCUGAAGAUGGUCAAGCGGC
AGAGCUACGGCACCAAUUCUGGCGUGGCCAUCUACCACAGAUACAAGGCC
GGCAACUACAGAAGCCCUCCUAUCACCGCCGACAUCGAGCUGGCUCUGCU
GAGAUGA

Hence, preferably the RNA construct comprises a sequence substantially as set out in SEQ ID No:456, or a fragment or variant thereof.

The at least one innate inhibitor protein (IIP) may not be selected from a group of IIPs consisting of: ORF4a (NS4a) of any coronavirus, ORF3b of any coronavirus, or the nucleocapsid proteins of mouse hepatitis virus and SARS (coronavirus); and orthologues thereof.

The at least one innate inhibitor protein (IIP) may not be selected from a group of IIPs consisting of: HSV-2 Us1; HSV-1 Us1; HSV-1Us11; ORF V20.0L; BVDV Npro; Langat NS5; Influenza NS1; PIV-5 V; SARS-CoV-2 ORF3b; and MERS-CoV ORF4a.

The RNA construct comprises a nucleotide sequence which encodes the at least one therapeutic biomolecule. This is referred to as the gene of interest (GOI) in FIG. 1.

The at least one therapeutic biomolecule may comprise a therapeutic protein. The skilled person would understand that therapeutic protein relates to any protein that has therapeutic application, preferably in human. Exemplary therapeutic biomolecules that can be encoded by the RNA molecule include proteins or peptides derived from pathogens, such as bacteria, viruses, fungi, protozoa/or parasites. The protein or peptide may be an antigen, and therefore one which may stimulate or trigger and immune response in the host. Hence, in the embodiment in which the at least one therapeutic biomolecule is an antigen, the RNA construct of the first aspect may be regarded as a vaccine.

The protein or peptide derived from a virus may be a viral antigen. The viral antigen may be derived from a virus selected from the group consisting of: Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainkuenza virus types 1-4 (PIV), Mumps, Sendai viruses, Simian virus 5, Bovine parainkuenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS CoV-1, SARS-CoV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses.

The protein or peptide derived from bacteria may be a bacterial antigen.

The bacterial antigen may derived from a bacterium selected from the group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae, Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcusfaecalis, Enterococcus faecium, Helicobacter pylori, Staphylococcus saprophyticus, Yersinia enter ocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp.

The protein or peptide derived from a fungus may be a fungal antigen.

The fungal antigen may be derived from a fungus selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisiae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp. The protein or peptide derived from a protozoan may be a protozoan antigen.

The protozoan antigen may be derived from a protozoan selected from the group consisting of: Entamoeba histolytica, Giardia lambli, Cryptosporidium parvum, Cyclospora cayatanensis and Toxoplasma.

The therapeutic biomolecule may be a protein or peptide derived from a plant. Preferably, the protein or peptide is a plant antigen. For example, the plant antigen may be derived from Ricinus communis.

In another embodiment, the therapeutic biomolecule may be an immunogen or an antigen. Preferably, the immunogen or an antigen is a tumour immunogen or antigen, or cancer immunogen or antigen. The tumour immunogens and antigens may be peptide-containing tumour antigens, such as a polypeptide tumour antigen or glycoprotein tumour antigens.

The tumour antigens may be (a) full length molecules associated with cancer cells, (b) homologs and modified forms of the same, including molecules with deleted, added and/or substituted portions, and (c) fragments of the same.

Suitable tumour immunogens include: class I-restricted antigens recognized by CD 8+ lymphocytes or class II-restricted antigens recognized by CD4+ lymphocytes.

The tumour antigen may be an antigen that is associated with a cancer selected from the group consisting of: a testis cancer, melanoma, lung cancer, head and neck cancer, NSCLC, breast cancer, gastrointestinal cancer, bladder cancer, colorectal cancer, pancreatic cancer, lymphoma, leukaemia, renal cancer, hepatoma, ovarian cancer, gastric cancer and prostate cancer.

The tumour antigen may be selected from:
(a) cancer-testis antigens, such as NY-ESO-I, SSX2, SCP-1, as well as RAGE, BAGE, GAGE and MAGE family polypeptides, for example, GAGE-I, GAGE-2, MAGE-I, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours);
(b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUM-1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT;
(c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukaemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer);
(d) shared antigens, for example, melanoma-melanocyte differentiation antigens, such as MART-1/Melan A, gplOO, MClR, melanocyte-stimulating hormone receptor, tyrosinase, tyrosinase related protein-1/TRPl and tyrosinase related protein-2/TRP2 (associated with, e.g., melanoma);
(e) prostate-associated antigens, such as PAP, PSA, PSMA, PSH-Pl, PSM-Pl, PSM-P2, associated with e.g., prostate cancer; and/or
(f) immunoglobulin idiotypes (associated with myeloma and B cell lymphomas, for example).

The therapeutic biomolecule may be a eukaryotic protein or peptide. In one embodiment, the eukaryotic protein or peptide is a mammalian protein or peptide. The mammalian protein or peptide may be selected from the group consisting of: an enzyme; an enzyme inhibitor; a hormone; an immune system protein; a receptor; a binding protein; a transcription factor; translation factor; tumour growth suppressing protein; a structural protein; and a blood protein.

The immune system protein may be an antibody or antigen binding fragment thereof. Accordingly, the therapeutic biomolecule may be an antibody or antigen binding fragment thereof. The antigen binding fragment may comprise an individual heavy or light chain, or a fragment thereof, such as VL, VH and Fd; a monovalent fragment, such as Fv, Fab, and Fab′; a bivalent fragment, such as F(ab′)₂; a single chain Fv (scFv); one or more complementarity determining region (CDR); or a Fc fragment.

The enzyme may be selected from the group consisting of: chymosin; gastric lipase; tissue plasminogen activator; streptokinase; a cholesterol biosynthetic or degradative steriodogenic enzyme; kinases; phosphodiesterases; methylases; de-methylases; dehydrogenases; cellulases; proteases; lipases; phospholipases; aromatases; cytochromes; adenylate or guanylaste cyclases and neuramidases.

The enzyme inhibitor may be tissue inhibitor of metalloproteinase (TIMP). The hormone may be growth hormone.

The immune system protein may be selected from the group consisting of: a cytokine; a chemokine; a lymphokine; erythropoietin; an integrin; addressin; selectin; homing receptors; T cell receptors and immunoglobulins.

The cytokine may be an interleukin, for example IL-2, IL-4 and/or IL-6, colony stimulating factor (CSF), granulocyte colony stimulating factor (G-CSF), granulocyte-macrophage colony stimulating factor (GM-CSF) or tumour necrosis factor (TNF).

The chemokine may be a macrophage inflammatory protein-2 and/or a plasminogen activator.

The lymphokine may be an interferon.

The immunoglobulin may be a natural, modified or chimeric immunoglobulin or a fragment thereof. Preferably, the immunoglobulin is a chimeric immunoglobulin having dual activity such as antibody enzyme or antibody-toxin chimera.

The hormone may be selected from the group consisting of: insulin, thyroid hormone, catecholamines, gonadotrophines, trophic hormones, prolactin, oxytocin, dopamine, bovine somatotropin, leptins; growth hormones (e.g., human growth hormone), growth factors (e.g., epidermal growth factor, nerve growth factor, insulin-like growth factor and the like).

The receptor may be a steroid hormone receptor or a peptide receptor. Preferably, the receptor is a growth factor receptor.

The binding protein may be a growth factor binding protein.

The tumour growth suppressing protein may be a protein that inhibits angiogenesis.

The structural protein may be selected from the group consisting of: collagen; fibroin; fibrinogen; elastin; tubulin; actin; and myosin.

The blood protein may be selected from the group consisting of thrombin; serum albumin; Factor VII; Factor VIII; insulin; Factor IX; Factor X; tissue plasminogen activator; protein C; von Willebrand factor; antithrombin III; glucocerebrosidase; erythropoietin granulocyte colony stimulating factor (GCSF) or modified Factor VIII; and anticoagulants.

In one preferred embodiment, the therapeutic biomolecule is a cytokine which is capable of regulating lymphoid homeostasis, preferably a cytokine which is involved in and preferably induces or enhances development, priming, expansion, differentiation and/or survival of T cells. Thus, preferably, the cytokine is an interleukin. Most preferably, IL-2, IL-7, IL-12, IL-15, or IL-21.

The therapeutic biomolecule may be protein that is capable of enhancing reprogramming of somatic cells to cells having stem cell characteristics. The protein that is capable of enhancing reprogramming of somatic cells to cells having stem cell characteristics may be selected from the group consisting of: OCT4, SOX2, NANOG, LIN28, p53, ART-4, BAGE, ss-catenin/m, Bcr-abL CAMEL, CAP-1, CASP-8, CDC27/m, CD 4/m, CEA, CLAUDIN-12, c-MYC, CT, Cyp-B, DAM, ELF2M, ETV6-AML1, G250, GAGE, GnT-V, GaplOO, HAGE, HER-2/neu, HPV-E7, HPV-E6, HAST-2, hTERT (or hTRT), LAGE, LDLR/FUT, MAGE-A, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Plac-1, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

Preferably, MAGE-A is selected from the group consisting of: MAGE-A 1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A 12. Preferably, the protein that is capable of enhancing reprogramming of somatic cells to cells having stem cell characteristics is OCT4, SOX2, LF4; c-MYC; NANOG; LIN28.

The therapeutic biomolecule may be a biomolecule that is utilised for the modification of cells ex vivo for cell-therapy indications. Thus, preferably the therapeutic biomolecule may be selected from the group consisting of an immunoglobulin, a T-cell receptor and NK receptor.

The therapeutic biomolecule may be an RNA molecule that is capable of regulating expression of endogenous host genes, for example an interfering RNA, such as small RNA, siRNA or microRNA.

The sequence encoding the at least one viral innate inhibitor protein (IIP) may be disposed anywhere within the RNA construct of the first aspect, such that the sequence encoding the therapeutic biomolecule (i.e. the GOI in FIG. 1) may be disposed either 5′ or 3′ to the sequence encoding the at least one IIP.

For example, in one embodiment, the sequence encoding the therapeutic biomolecule is preferably disposed 5′ to the sequence encoding the at least one innate modulatory protein. See for example, the saRNA embodiments 2a, 3a, 4a, and the mRNA embodiments 6a and 7a shown in FIG. 1.

However, in another embodiment, the sequence encoding the therapeutic biomolecule is preferably disposed 3′ to the sequence encoding the at least one innate modulatory protein. See for example, the saRNA embodiments 2b, 3b, 4b, and the mRNA embodiments 6b and 7b shown in FIG. 1.

Preferably, the RNA construct according to the first aspect comprises at least one promotor, which may be either genomic or subgenomic. Preferably, however, the promoter is a subgenomic promoter, as is shown in FIG. 1 (embodiments 1-4b).

Preferably, therefore, saRNA constructs of the invention comprise a promoter. The skilled person would understand that the subgenomic promotor relates to a promoter that is operably linked to the sequences encoding the at least one therapeutic biomolecule and the at least one innate inhibitor protein, such that it enables the transcription of the nucleotide sequence encoding the therapeutic biomolecule and the at least one innate modulatory protein.

Preferably, the subgenomic promoter is 26S, which is provided herein as SEQ ID No: 67, as follows:

[SEQ ID No: 67]
GGGCCCCTATAACTCTCTACGGCTAACCTGAATGGACTACGACAT

Accordingly, preferably the promoter (which is preferably a subgenomic promoter) is as substantially as set out in SEQ ID NO: 67, or a variant or fragment thereof.

In one embodiment, the same promotor is operably linked to the sequence encoding the at least therapeutic biomolecule and the sequence encoding the at least one innate modulatory protein.

The inventor's designs, wherein both the therapeutic biomolecule (i.e. GOI) and IIP are encoded by a single strand of RNA, advantageously enables the use of much smaller doses of RNA, because it ensures that the protein is being expressed in the same cell that is sensing the RNA, and can also be replicated, therefore having the additional aspect of expression and amplification of the innate modulatory component.

Thus, in one embodiment of the RNA construct, the promoter is disposed 5′ of the sequence encoding the at least one therapeutic biomolecule and the sequence encoding the at least one innate inhibitor protein, such that the promoter is operably linked to both sequences, thereby driving expression of both.

In another embodiment, however, a first promotor is operably linked to the sequence encoding the at least one therapeutic biomolecule, and a second promotor is operably linked the sequence encoding the at least one innate inhibitor protein. This is referred to as a double genomic construct. Preferably, the first and/or second promoter is genomic or subgenomic. Preferably, both promoters are subgenomic promoters, such as 26S.

The RNA construct may encode at least two, three, four or five IIPs. In embodiments in which there is more than one sequence encoding an innate modulatory protein, a single promotor may be operably linked to all sequences encoding an innate modulatory protein.

Alternatively, a promotor may be linked to each of the sequences encoding an innate modulatory protein, such that each innate modulatory protein is operably linked to a separate promoter. In this embodiment, the separate promoters may comprise the same promotor sequence or different promoter sequences. In another embodiment, different promotors are operably linked to each sequence encoding an innate modulatory protein.

The RNA construct may further comprise a linker sequence disposed between the sequence encoding the at least one therapeutic biomolecule and the sequence encoding the at least one innate modulatory protein. This linker sequence is such that it allows the production of the IIP and the production of the therapeutic molecule from the single promoter. In one embodiment, the linker sequence encodes a peptide linker that is configured to be digested or cleaved following translation, to thereby separate the at least one therapeutic biomolecule and the at least one innate modulatory protein in the host cell. As such, the linker sequence is preferably a cleavable peptide, which may form a cleavage site, for example a 2A peptide (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873).

Preferably, the linker sequence encoding the 2A peptide sequence connects the two coding sequence together. This enables the RNA construct to overcome the size restrictions that may occur with expression in various vectors and enables expression and translation of all of the peptides encoded by the RNA construct of the first aspect to occur under control of a single promoter, as a single protein. Thus, following the translation of the single protein comprising the sequences of the IIP, the 2A peptide, and the therapeutic biomolecule, cleavage occurs in the viral 2A peptide sequence at the terminal glycine-proline link, thereby liberating two polypeptides.

The 2A spacer sequence may be any known variant, which includes those sequences referred to as E2A, F2A, P2A and T2A, as disclosed in Wang Y et al. Scientific Reports 2015, 5, i.e. suitable 2A peptides include the porcine teschovirus-1 2A (P2A)—ATNFSLLKQAGDVEENPGP (SEQ ID No: 68), Thosea asigna virus 2A (T2A)—QCTNYALLKLAGDVESNPGP (SEQ ID No: 69), equine rhinitis A virus 2A (E2A), and Foot and mouth disease virus 2A (F2A) VKQTLNFDLLKLAGDVESNPGP (SEQ ID No: 70). Preferably, the 2A peptide is Thosea asigna virus 2A (T2A).

In another embodiment, the cleavable peptide is a self-cleaving peptide. In an embodiment, the linker comprises a viral 2A peptide spacer and further comprises a furin cleavage site. Preferably, the self-cleaving peptide is a furin/2A peptide. Insertion of an upstream furin cleavage site allows the removal of 2A residues that would otherwise remain attached to the upstream protein.

The furin sequence may be disposed 3′ or 5′ of the 2A sequence. Preferably, however, the furin sequence is disposed 5′ of the 2A sequence, and preferably with a GSG spacer disposed between the furin and 2A sequence.

The skilled person would appreciate that furin is a ubiquitous calcium-dependent proprotein convertase located in the secretory pathway (mainly in the golgi and trans-golgi network) that cleaves precursor proteins at a specific recognition sequence—canonically R-X-R/K/X-R (SEQ ID No: 71), and cleaving the proprotein after the final R. Thus, in one embodiment the furin sequence is R-X-R/K/X-R. However, preferably, the furin sequence is the optimised sequence RRRRRR (SEQ ID No: 72) a GSG sequence. Preferably, the GSG spacer is disposed 3′ of the furin sequence and 5′ of the 2A sequence.

Thus, preferably, the spacer sequence is the furin/T2A, as provided by NCBI Reference Sequence: GenBank: AAC97195.1, and provided herein as SEQ ID No: 73, as follows:

[SEQ ID No: 73]
RRRRRRGSGEGRGSLLTCGDVEENPGP

Hence, preferably the spacer sequence comprises an amino acid sequence substantially as set out in SEQ ID NO: 73, or a variant or fragment thereof.

FIG. 1 shows embodiments 2a, 2b and 6a, 6b in which the GOI and IIP are linked by a nucleotide sequence which encodes the Furin-T2a cleavage site. In one embodiment, shown as either 2a or 6a in FIG. 1, the F-T2a cleavage site separates a 5′ GOI and a 3′ IIP. In one embodiment, shown as either 2b or 6b in FIG. 1, the F-T2a cleavage site separates a 3′ GOI and a 5′ IIP.

In embodiments in which the RNA construct or replicon comprises more than one sequence encoding an innate modulatory protein, the construct may comprise linker sequences disposed between each sequence encoding an innate modulatory protein, or only between some IIPs.

In one embodiment, the sequence encoding the at least one therapeutic biomolecule and the sequence encoding the at least one innate modulatory protein may be separated by a stop codon followed by an internal ribosome entry site (IRES) sequence capable of initiating translation of the downstream sequence, whichever sequence that may be (i.e. GOI or IIP as shown in embodiments 3a, 3b, 7a or 7b in FIG. 1). Therefore, preferably the IRES sequence is disposed between the sequence encoding the at least one therapeutic biomolecule and the sequence encoding at least one innate modulatory protein. Where multiple sequences encoding at least one innate modulatory protein are used, linker sequences may include combinations of known cleavage sequences and/or IRES sequences. In one embodiment, shown as either 3a or 7a in FIG. 1, the IRES site separates a 5′ GOI and a 3′ IIP. In one embodiment, shown as either 3b or 7b in FIG. 1, the IRES site separates a 3′ GOI and a 5′ IIP.

In an embodiment, the IRES is a picornavirus IRES. Oher typical IRES sequences include those such as the IRES sequence of encephalomyocarditis virus (EMCV) or vascular endothelial growth factor and type 1 collagen-inducible protein (VCIP), and would be known to those skilled in the art.

In other embodiments, the IRES may be selected from a rhinovirus IRES, a hepatitis A virus IRES, a hepatitis C virus IRES, a poliovirus IRES, an enterovirus IRES, a cardiovirus IRES, an aphthovirus IRES, flavivirus IRES, a pestivirus IRES, a cripavirus IRES, a Rhopalosiphum padi virus IRES, or any suitable IRES. In particular, the IRES may be any IRES described by the “IRESite” which provides a database of experimentally verified IRES structures (http://www.iresite.org/), or as disclosed in “New Messenger RNA Research Communications” (ISBN: 1-60021-488-6).

In a preferred embodiment, the IRES is a foot-and-mouth disease virus (FMDV) IRES, which may be as set out in SEQ ID No:74, or a fragment or variant thereof, as follows:

[SEQ ID NO: 74]
AGCAGGTTTCCCCAACTGACACAAAACGTGCAACTTGAAACTCCGCCTGG
TCTTTCCAGGTCTAGAGGGGTAACACTTTGTACTGCGTTTGGCTCCACGC
TCGATCCACTGGCGAGTGTTAGTAACAGCACTGTTGCTTCGTAGCGGAGC
ATGACGGCCGTGGGAACTCCTCCTTGGTAACAAGGACCCACGGGGCCAAA
AGCCACGCCCACACGGGCCCGTCATGTGTGCAACCCCAGCACGGCGACTT
TACTGCGAAACCCACTTTAAAGTGACATTGAAACTGGTACCCACACACTG
GTGACAGGCTAAGGATGCCCTTCAGGTACCCCGAGGTAACACGCGACACT
CGGGATCTGAGAAGGGGACTGGGGCTTCTATAAAAGCGCTCGGTTTAAAA
AGCTTCTATGCCTGAATAGGTGACCGGAGGTCGGCACCTTTCCTTTGCAA
TTACTGACCAC

In another preferred embodiment, the IRES is an encephalomyocarditis virus (EMCV) IRES. The EMCV IRES may be as set out in SEQ ID No:75, or a fragment or variant thereof, as follows:

[SEQ ID NO: 75]
CGTTACTGGCCGAAGCCGCTTGGAATAAGGCCGGTGTGCGTTTGTCTATA
TGTTATTTTCCACCATATTGCCGTCTTTTGGCAATGTGAGGGCCCGGAAA
CCTGGCCCTGTCTTCTTGACGAGCATTCCTAGGGGTCTTTCCCCTCTCGC
CAAAGGAATGCAAGGTCTGTTGAATGTCGTGAAGGAAGCAGTTCCTCTGG
AAGCTTCTTGAAGACAAACAACGTCTGTAGCGACCCTTTGCAGGCAGCGG
AACCCCCCACCTGGCGACAGGTGCCTCTGCGGCCAAAAGCCACGTGTATA
AGATACACCTGCCAACAAGGGGCTGAAGGATGCCCAGAAGGTACCCCATT
GTATGGGATCTGATCTGGGGCCTCGGTGCACATGCTTTTCATGTGTTTAG
TCGAGGTTAAAAAACGTCTAGGCCCCCCGAACCACGGGGACGTGGTTTTC
CTTTGAAAAACACGATGATAATA

Therefore, preferably the IRES comprises a nucleotide sequence substantially as set out in SEQ ID No: 74 or 75, or a fragment or variant thereof.

Alternatively, instead of an IRES or a 2A linker, the linker sequence may comprise a sequence encoding a flexible linker, which allows for the expression of both the therapeutic biomolecule and IIP as a single polypeptide chain, but wherein the therapeutic biomolecule and IIP act as independent proteins. Hence, the proteins exert their effects in the same manner as if they were singly expressed.

The flexible linker sequence may be as disclosed by WO 2013/061076 A1 (Oxford Biomedica). The flexible linker sequence may be referred to herein as SEQ ID No:76, or a fragment or variant thereof, as follows:

[SEQ ID NO: 76]
GGAGGTGGCGGGTCCGGGGGCGGGGGTAGCGGTGGCGGGGGCTCC

Preferably, therefore, the flexible linker sequence comprises a nucleotide sequence substantially as set out in SEQ ID No: 76, or a fragment or variant thereof.

In one preferred embodiment, the flexible linker sequence comprises a nucleotide sequence encoding an amino acid sequence referred to herein as SEQ ID NO: 77, or a fragment or variant thereof, as set out below:

[SEQ ID NO: 77]
GGGGSGGGGSGGGGS

Preferably, therefore, the flexible linker sequence encodes an amino acid sequence substantially as set out in SEQ ID No: 77, or a fragment or variant thereof.

In yet another embodiment, the sequence encoding the at least one therapeutic biomolecule and the at least one innate inhibitor protein may be separated by a stop codon followed by a second subgenomic promotor sequence capable of initiating transcription of the downstream sequence. Examples of this embodiment are illustrated in FIG. 1, embodiments 4a and 4b.

The RNA construct (preferably when it is a saRNA construct) may encode at least one non-structural protein (NSP), disposed 5′ or 3′ of the sequence encoding the at least one therapeutic biomolecule and the at least one innate modulatory protein. Preferably, the sequence encoding the at least one NSP is disposed 5′ of the sequences encoding the therapeutic biomolecule and the at least one innate modulatory protein. Thus, preferably the sequence encoding the at least one NSP is disposed at the 5′ end of the RNA construct.

The at least one non-structural protein, which is encoded by the RNA construct, may be the RNA polymerase NSP4. The one or more non-structural protein preferably encodes a replicase. Preferably, the construct encodes NSP1, NSP2, NSP3 and NSP4. The skilled person would understand that NSP1 is the viral capping enzyme and membrane anchor of the replication complex (RC), while NSP2 is an RNA helicase and the protease responsible for the ns polyprotein processing. NSP3 interacts with several host proteins and may modulate protein poly- and mono-ADP-ribosylation, and NSP4 is the core viral RNA-dependent RNA polymerase.

In one embodiment, NSP1 is provided herein as SEQ ID No: 78, as follows:

[SEQ ID No: 78]
MEKVHVDIEEDSPFLRALQRSFPQFEVEAKQVTDNDHANARAFSHLASKLI
ETEVDPSDTILDIGSAPARRMYSKHKYHCICPMRCAEDPDRLYKYATKLKK
NCKEITDKELDKKMKELAAVMSDPDLETETMCLHDDESCRYEGQVAVYQDV
YAVDGPTSLYHQANKGVRVAYWIGFDTTPFMFKNLAGAYPSYSTNWADETV
LTARNIGLCSSDVMERSRRGMSILRKKYLKPSNNVLFSVGSTIYHEKRDLL
RSWHLPSVFHLRGKQNYTCRCETIVSCDGYVVKRIAISPGLYGKPSGYAAT
MHREGFFARWAKEYKEDQEDERPLGLRDRQLVMGCCWAFRRHKITSIYKRP
DTQTIIKVNSDFHSFVLPRIGSNTLEIGLRTRIRKMLEEHKEPSPLITAED
VQEAKCAADEAKEVREAEELRAALPPLAADVEEPTLEADVDLMLQEAGA

Accordingly, NSP1 preferably comprises an amino acid sequence as substantially as set out in SEQ ID No: 78, or a biologically active variant or fragment thereof.

In one embodiment, NSP1 is encoded by a nucleotide sequence a defined in SEQ ID No: 79, as follows:

[SEQ ID No: 79]
ATGGAGAAAGTTCACGTTGACATCGAGGAAGACAGCCCATTCCTCAGAGCT
TTGCAGCGGAGCTTCCCGCAGTTTGAGGTAGAAGCCAAGCAGGTCACTGAT
AATGACCATGCTAATGCCAGAGCGTTTTCGCATCTGGCTTCAAAACTGATC
GAAACGGAGGTGGACCCATCCGACACGATCCTTGACATTGGAAGTGCGCCC
GCCCGCAGAATGTATTCTAAGCACAAGTATCATTGTATCTGTCCGATGAGA
TGTGCGGAAGATCCGGACAGATTGTATAAGTATGCAACTAAGCTGAAGAAA
AACTGTAAGGAAATAACTGATAAGGAATTGGACAAGAAAATGAAGGAGCTG
GCCGCCGTCATGAGCGACCCTGACCTGGAAACTGAGACTATGTGCCTCCAC
GACGACGAGTCGTGTCGCTACGAAGGGCAAGTCGCTGTTTACCAGGATGTA
TACGCGGTTGACGGACCGACAAGTCTCTATCACCAAGCCAATAAGGGAGTT
AGAGTCGCCTACTGGATAGGCTTTGACACCACCCCTTTTATGTTTAAGAAC
TTGGCTGGAGCATATCCATCATACTCTACCAACTGGGCCGACGAAACCGTG
TTAACGGCTCGTAACATAGGCCTATGCAGCTCTGACGTTATGGAGCGGTCA
CGTAGAGGGATGTCCATTCTTAGAAAGAAGTATTTGAAACCATCCAACAAT
GTTCTATTCTCTGTTGGCTCGACCATCTACCACGAGAAGAGGGACTTACTG
AGGAGCTGGCACCTGCCGTCTGTATTTCACTTACGTGGCAAGCAAAATTAC
ACATGTCGGTGTGAGACTATAGTTAGTTGCGACGGGTACGTCGTTAAAAGA
ATAGCTATCAGTCCAGGCCTGTATGGGAAGCCTTCAGGCTATGCTGCTACG
ATGCACCGCGAGGGATTCTTGTGCTGCAAAGTGACAGACACATTGAACGGG
GAGAGGGTCTCTTTTCCCGTGTGCACGTATGTGCCAGCTACATTGTGTGAC
CAAATGACTGGCATACTGGCAACAGATGTCAGTGCGGACGACGCGCAAAAA
CTGCTGGTTGGGCTCAACCAGCGTATAGTCGTCAACGGTCGCACCCAGAGA
AACACCAATACCATGAAAAATTACCTTTTGCCCGTAGTGGCCCAGGCATTT
GCTAGGTGGGCAAAGGAATATAAGGAAGATCAAGAAGATGAAAGGCCACTA
GGACTACGAGATAGACAGTTAGTCATGGGGTGTTGTTGGGCTTTTAGAAGG
CACAAGATAACATCTATTTATAAGCGCCCGGATACCCAAACCATCATCAAA
GTGAACAGCGATTTCCACTCATTCGTGCTGCCCAGGATAGGCAGTAACACA
TTGGAGATCGGGCTGAGAACAAGAATCAGGAAAATGTTAGAGGAGCACAAG
GAGCCGTCACCTCTCATTACCGCCGAGGACGTACAAGAAGCTAAGTGCGCA
GCCGATGAGGCTAAGGAGGTGCGTGAAGCCGAGGAGTTGCGCGCAGCTCTA
CCACCTTTGGCAGCTGATGTTGAGGAGCCCACTCTGGAAGCCGATGTCGAC
TTGATGTTACAAGAGGCTGGGGCC

Accordingly, NSP1 is preferably encoded by a nucleotide sequence as substantially as set out in SEQ ID No: 79, or a variant or fragment thereof.

Accordingly, therefore, preferably the RNA construct comprises an RNA nucleotide sequence substantially as set out as SEQ ID No: 80, or a variant or fragment thereof.

[SEQ ID No: 80]
AUGGAGAAAGUUCACGUUGACAUCGAGGAAGACAGCCCAUUCCUCAGAGCUUUGCAGCGGAGCUUCCCGCAGUUUGAG
GUAGAAGCCAAGCAGGUCACUGAUAAUGACCAUGCUAAUGCCAGAGCGUUUUCGCAUCUGGCUUCAAAACUGAUCGAA
ACGGAGGUGGACCCAUCCGACACGAUCCUUGACAUUGGAAGUGCGCCCGCCCGCAGAAUGUAUUCUAAGCACAAGUAU
CAUUGUAUCUGUCCGAUGAGAUGUGCGGAAGAUCCGGACAGAUUGUAUAAGUAUGCAACUAAGCUGAAGAAAAACUGU
AAGGAAAUAACUGAUAAGGAAUUGGACAAGAAAAUGAAGGAGCUGGCCGCCGUCAUGAGCGACCCUGACCUGGAAACU
GAGACUAUGUGCCUCCACGACGACGAGUCGUGUCGCUACGAAGGGCAAGUCGCUGUUUACCAGGAUGUAUACGCGGUU
GACGGACCGACAAGUCUCUAUCACCAAGCCAAUAAGGGAGUUAGAGUCGCCUACUGGAUAGGCUUUGACACCACCCCU
UUUAUGUUUAAGAACUUGGCUGGAGCAUAUCCAUCAUACUCUACCAACUGGGCCGACGAAACCGUGUUAACGGCUCGU
AACAUAGGCCUAUGCAGCUCUGACGUUAUGGAGCGGUCACGUAGAGGGAUGUCCAUUCUUAGAAAGAAGUAUUUGAAA
CCAUCCAACAAUGUUCUAUUCUCUGUUGGCUCGACCAUCUACCACGAGAAGAGGGACUUACUGAGGAGCUGGCACCUG
CCGUCUGUAUUUCACUUACGUGGCAAGCAAAAUUACACAUGUCGGUGUGAGACUAUAGUUAGUUGCGACGGGUACGUC
GUUAAAAGAAUAGCUAUCAGUCCAGGCCUGUAUGGGAAGCCUUCAGGCUAUGCUGCUACGAUGCACCGCGAGGGAUUC
UUGUGCUGCAAAGUGACAGACACAUUGAACGGGGAGAGGGUCUCUUUUCCCGUGUGCACGUAUGUGCCAGCUACAUUG
UGUGACCAAAUGACUGGCAUACUGGCAACAGAUGUCAGUGCGGACGACGCGCAAAAACUGCUGGUUGGGCUCAACCAG
CGUAUAGUCGUCAACGGUCGCACCCAGAGAAACACCAAUACCAUGAAAAAUUACCUUUUGCCCGUAGUGGCCCAGGCA
UUUGCUAGGUGGGCAAAGGAAUAUAAGGAAGAUCAAGAAGAUGAAAGGCCACUAGGACUACGAGAUAGACAGUUAGUC
AUGGGGUGUUGUUGGGCUUUUAGAAGGCACAAGAUAACAUCUAUUUAUAAGCGCCCGGAUACCCAAACCAUCAUCAAA
GUGAACAGCGAUUUCCACUCAUUCGUGCUGCCCAGGAUAGGCAGUAACACAUUGGAGAUCGGGCUGAGAACAAGAAUC
AGGAAAAUGUUAGAGGAGCACAAGGAGCCGUCACCUCUCAUUACCGCCGAGGACGUACAAGAAGCUAAGUGCGCAGCC
GAUGAGGCUAAGGAGGUGCGUGAAGCCGAGGAGUUGCGCGCAGCUCUACCACCUUUGGCAGCUGAUGUUGAGGAGCCC
ACUCUGGAAGCCGAUGUCGACUUGAUGUUACAAGAGGCUGGGGCC

In one embodiment, NSP2 is provided herein as SEQ ID No: 81, as follows:

[SEQ ID No: 81]
GSVETPRGLIKVTSYDGEDKIGSYAVLSPQAVLKSEKLSCIHPLAEQVIVITHSGRKGRYAVEPYHGKVVVPEGHAIP
VQDFQALSESATIVYNEREFVNRYLHHIATHGGALNTDEEYYKTVKPSEHDGEYLYDIDRKQCVKKELVTGLGLTGEL
VDPPFHEFAYESLRTRPAAPYQVPTIGVYGVPGSGKSGIIKSAVTKKDLVVSAKKENCAEIIRDVKKMKGLDVNARTV
DSVLLNGCKHPVETLYIDEAFACHAGTLRALIAIIRPKKAVLCGDPKQCGFFNMMCLKVHFNHEICTQVFHKSISRRC
TKSVTSVVSTLFYDKKMRTTNPKETKIVIDTTGSTKPKQDDLILTCFRGWVKQLQIDYKGNEIMTAAASQGLTRKGVY
AVRYKVNENPLYAPTSEHVNVLLTRTEDRIVWKTLAGDPWIKTLTAKYPGNFTATIEEWQAEHDAIMRHILERPDPTD
VFQNKANVCWAKALVPVLKTAGIDMTTEQWNTVDYFETDKAHSAEIVLNQLCVRFFGLDLDSGLFSAPTVPLSIRNNH
WDNSPSPNMYGLNKEVVRQLSRRYPQLPRAVATGRVYDMNTGTLRNYDPRINLVPVNRRLPHALVLHHNEHPQSDFSS
FVSKLKGRTVLVVGEKLSVPGKMVDWLSDRPEATFRARLDLGIPGDVPKYDIIFVNVRTPYKYHHYQQCEDHAIKLSM
LTKKACLHLNPGGTCVSIGYGYADRASESIIGAIARQFKFSRVCKPKSSLEETEVLFVFIGYDRKARTHNSYKLSSTL
TNIYTGSRLHEAGC

Accordingly, NSP2 preferably comprises an amino acid sequence as substantially as set out in SEQ ID No: 81, or a biologically active variant or fragment thereof.

In one embodiment, NSP2 is encoded by a nucleotide sequence a defined in SEQ ID No: 82, as follows:

[SEQ ID No: 82]
GGCTCAGTGGAGACACCTCGTGGCTTGATAAAGGTTACCAGCTACGATGGCGAGGACAAGATCGGCTCTTACGCTGTG
CTTTCTCCGCAGGCTGTACTCAAGAGTGAAAAATTATCTTGCATCCACCCTCTCGCTGAACAAGTCATAGTGATAACA
CACTCTGGCCGAAAAGGGCGTTATGCCGTGGAACCATACCATGGTAAAGTAGTGGTGCCAGAGGGACATGCAATACCC
GTCCAGGACTTTCAAGCTCTGAGTGAAAGTGCCACCATTGTGTACAACGAACGTGAGTTCGTAAACAGGTACCTGCAC
CATATTGCCACACATGGAGGAGCGCTGAACACTGATGAAGAATATTACAAAACTGTCAAGCCCAGCGAGCACGACGGC
GAATACCTGTACGACATCGACAGGAAACAGTGCGTCAAGAAAGAACTAGTCACTGGGCTAGGGCTCACAGGCGAGCTG
GTGGATCCTCCCTTCCATGAATTCGCCTACGAGAGTCTGAGAACACGACCAGCCGCTCCTTACCAAGTACCAACCATA
GGGGTGTATGGCGTGCCAGGATCAGGCAAGTCTGGCATCATTAAAAGCGCAGTCACCAAAAAAGATCTAGTGGTGAGC
GCCAAGAAAGAAAACTGTGCAGAAATTATAAGGGACGTCAAGAAAATGAAAGGGCTGGACGTCAATGCCAGAACTGTG
GACTCAGTGCTCTTGAATGGATGCAAACACCCCGTAGAGACCCTGTATATTGACGAAGCTTTTGCTTGTCATGCAGGT
ACTCTCAGAGCGCTCATAGCCATTATAAGACCTAAAAAGGCAGTGCTCTGCGGGGATCCCAAACAGTGCGGTTTTTTT
AACATGATGTGCCTGAAAGTGCATTTTAACCACGAGATTTGCACACAAGTCTTCCACAAAAGCATCTCTCGCCGTTGC
ACTAAATCTGTGACTTCGGTCGTCTCAACCTTGTTTTACGACAAAAAAATGAGAACGACGAATCCGAAAGAGACTAAG
ATTGTGATTGACACTACCGGCAGTACCAAACCTAAGCAGGACGATCTCATTCTCACTTGTTTCAGAGGGTGGGTGAAG
CAGTTGCAAATAGATTACAAAGGCAACGAAATAATGACGGCAGCTGCCTCTCAAGGGCTGACCCGTAAAGGTGTGTAT
GCCGTTCGGTACAAGGTGAATGAAAATCCTCTGTACGCACCCACCTCAGAACATGTGAACGTCCTACTGACCCGCACG
GAGGACCGCATCGTGTGGAAAACACTAGCCGGCGACCCATGGATAAAAACACTGACTGCCAAGTACCCTGGGAATTTC
ACTGCCACGATAGAGGAGTGGCAAGCAGAGCATGATGCCATCATGAGGCACATCTTGGAGAGACCGGACCCTACCGAC
GTCTTCCAGAATAAGGCAAACGTGTGTTGGGCCAAGGCTTTAGTGCCGGTGCTGAAGACCGCTGGCATAGACATGACC
ACTGAACAATGGAACACTGTGGATTATTTTGAAACGGACAAAGCTCACTCAGCAGAGATAGTATTGAACCAACTATGC
GTGAGGTTCTTTGGACTCGATCTGGACTCCGGTCTATTTTCTGCACCCACTGTTCCGTTATCCATTAGGAATAATCAC
TGGGATAACTCCCCGTCGCCTAACATGTACGGGCTGAATAAAGAAGTGGTCCGTCAGCTCTCTCGCAGGTACCCACAA
CTGCCTCGGGCAGTTGCCACTGGAAGAGTCTATGACATGAACACTGGTACACTGCGCAATTATGATCCGCGCATAAAC
CTAGTACCTGTAAACAGAAGACTGCCTCATGCTTTAGTCCTCCACCATAATGAACACCCACAGAGTGACTTTTCTTCA
TTCGTCAGCAAATTGAAGGGCAGAACTGTCCTGGTGGTCGGGGAAAAGTTGTCCGTCCCAGGCAAAATGGTTGACTGG
TTGTCAGACCGGCCTGAGGCTACCTTCAGAGCTCGGCTGGATTTAGGCATCCCAGGTGATGTGCCCAAATATGACATA
ATATTTGTTAATGTGAGGACCCCATATAAATACCATCACTATCAGCAGTGTGAAGACCATGCCATTAAGCTTAGCATG
TTGACCAAGAAAGCTTGTCTGCATCTGAATCCCGGCGGAACCTGTGTCAGCATAGGTTATGGTTACGCTGACAGGGCC
AGCGAAAGCATCATTGGTGCTATAGCGCGGCAGTTCAAGTTTTCCCGGGTATGCAAACCGAAATCCTCACTTGAAGAG
ACGGAAGTTCTGTTTGTATTCATTGGGTACGATCGCAAGGCCCGTACGCACAATTCTTACAAGCTTTCATCAACCTTG
ACCAACATTTATACAGGTTCCAGACTCCACGAAGCCGGATGT

Accordingly, preferably NSP2 is encoded by a nucleotide sequence as substantially as set out in SEQ ID No: 82, or a variant or fragment thereof.

Thus, the RNA construct may comprise SEQ ID No: 83, as follows:

[SEQ ID No: 83]
GGCUCAGUGGAGACACCUCGUGGCUUGAUAAAGGUUACCAGCUACGAUGGCGAGGACAAGAUCGGCUCUUACGCUGUG
CUUUCUCCGCAGGCUGUACUCAAGAGUGAAAAAUUAUCUUGCAUCCACCCUCUCGCUGAACAAGUCAUAGUGAUAACA
CACUCUGGCCGAAAAGGGCGUUAUGCCGUGGAACCAUACCAUGGUAAAGUAGUGGUGCCAGAGGGACAUGCAAUACCC
GUCCAGGACUUUCAAGCUCUGAGUGAAAGUGCCACCAUUGUGUACAACGAACGUGAGUUCGUAAACAGGUACCUGCAC
CAUAUUGCCACACAUGGAGGAGCGCUGAACACUGAUGAAGAAUAUUACAAAACUGUCAAGCCCAGCGAGCACGACGGC
GAAUACCUGUACGACAUCGACAGGAAACAGUGCGUCAAGAAAGAACUAGUCACUGGGCUAGGGCUCACAGGCGAGCUG
GUGGAUCCUCCCUUCCAUGAAUUCGCCUACGAGAGUCUGAGAACACGACCAGCCGCUCCUUACCAAGUACCAACCAUA
GGGGUGUAUGGCGUGCCAGGAUCAGGCAAGUCUGGCAUCAUUAAAAGCGCAGUCACCAAAAAAGAUCUAGUGGUGAGC
GCCAAGAAAGAAAACUGUGCAGAAAUUAUAAGGGACGUCAAGAAAAUGAAAGGGCUGGACGUCAAUGCCAGAACUGUG
GACUCAGUGCUCUUGAAUGGAUGCAAACACCCCGUAGAGACCCUGUAUAUUGACGAAGCUUUUGCUUGUCAUGCAGGU
ACUCUCAGAGCGCUCAUAGCCAUUAUAAGACCUAAAAAGGCAGUGCUCUGCGGGGAUCCCAAACAGUGCGGUUUUUUU
AACAUGAUGUGCCUGAAAGUGCAUUUUAACCACGAGAUUUGCACACAAGUCUUCCACAAAAGCAUCUCUCGCCGUUGC
ACUAAAUCUGUGACUUCGGUCGUCUCAACCUUGUUUUACGACAAAAAAAUGAGAACGACGAAUCCGAAAGAGACUAAG
AUUGUGAUUGACACUACCGGCAGUACCAAACCUAAGCAGGACGAUCUCAUUCUCACUUGUUUCAGAGGGUGGGUGAAG
CAGUUGCAAAUAGAUUACAAAGGCAACGAAAUAAUGACGGCAGCUGCCUCUCAAGGGCUGACCCGUAAAGGUGUGUAU
GCCGUUCGGUACAAGGUGAAUGAAAAUCCUCUGUACGCACCCACCUCAGAACAUGUGAACGUCCUACUGACCCGCACG
GAGGACCGCAUCGUGUGGAAAACACUAGCCGGCGACCCAUGGAUAAAAACACUGACUGCCAAGUACCCUGGGAAUUUC
ACUGCCACGAUAGAGGAGUGGCAAGCAGAGCAUGAUGCCAUCAUGAGGCACAUCUUGGAGAGACCGGACCCUACCGAC
GUCUUCCAGAAUAAGGCAAACGUGUGUUGGGCCAAGGCUUUAGUGCCGGUGCUGAAGACCGCUGGCAUAGACAUGACC
ACUGAACAAUGGAACACUGUGGAUUAUUUUGAAACGGACAAAGCUCACUCAGCAGAGAUAGUAUUGAACCAACUAUGC
GUGAGGUUCUUUGGACUCGAUCUGGACUCCGGUCUAUUUUCUGCACCCACUGUUCCGUUAUCCAUUAGGAAUAAUCAC
UGGGAUAACUCCCCGUCGCCUAACAUGUACGGGCUGAAUAAAGAAGUGGUCCGUCAGCUCUCUCGCAGGUACCCACAA
CUGCCUCGGGCAGUUGCCACUGGAAGAGUCUAUGACAUGAACACUGGUACACUGCGCAAUUAUGAUCCGCGCAUAAAC
CUAGUACCUGUAAACAGAAGACUGCCUCAUGCUUUAGUCCUCCACCAUAAUGAACACCCACAGAGUGACUUUUCUUCA
UUCGUCAGCAAAUUGAAGGGCAGAACUGUCCUGGUGGUCGGGGAAAAGUUGUCCGUCCCAGGCAAAAUGGUUGACUGG
UUGUCAGACCGGCCUGAGGCUACCUUCAGAGCUCGGCUGGAUUUAGGCAUCCCAGGUGAUGUGCCCAAAUAUGACAUA
AUAUUUGUUAAUGUGAGGACCCCAUAUAAAUACCAUCACUAUCAGCAGUGUGAAGACCAUGCCAUUAAGCUUAGCAUG
UUGACCAAGAAAGCUUGUCUGCAUCUGAAUCCCGGCGGAACCUGUGUCAGCAUAGGUUAUGGUUACGCUGACAGGGCC
AGCGAAAGCAUCAUUGGUGCUAUAGCGCGGCAGUUCAAGUUUUCCCGGGUAUGCAAACCGAAAUCCUCACUUGAAGAG
ACGGAAGUUCUGUUUGUAUUCAUUGGGUACGAUCGCAAGGCCCGUACGCACAAUUCUUACAAGCUUUCAUCAACCUUG
ACCAACAUUUAUACAGGUUCCAGACUCCACGAAGCCGGAUGU

Accordingly, therefore, preferably the RNA construct comprises an RNA nucleotide sequence substantially as set out as SEQ ID No: 83, or a variant or fragment thereof.

In one embodiment, NSP3 is provided herein as SEQ ID No: 84, as follows:

[SEQ ID No: 84]
APSYHVVRGDIATATEGVIINAANSKGQPGGGVCGALYKKFPESFDLQPIEVGKARLVKGAAKHIIHAVGPNFNKVSE
VEGDKQLAEAYESIAKIVNDNNYKSVAIPLLSTGIFSGNKDRLTQSLNHLLTALDTTDADVAIYCRDKKWEMTLKEAV
ARREAVEEICISDDSSVTEPDAELVRVHPKSSLAGRKGYSTSDGKTFSYLEGTKFHQAAKDIAEINAMWPVATEANEQ
VCMYILGESMSSIRSKCPVEESEASTPPSTLPCLCIHAMTPERVQRLKASRPEQITVCSSFPLPKYRITGVQKIQCSQ
PILFSPKVPAYIHPRKYLVETPPVDETPEPSAENQSTEGTPEQPPLITEDETRTRTPEPIIIEEEEEDSISLLSDGPT
HQVLQVEADIHGPPSVSSSSWSIPHASDFDVDSLSILDTLEGASVTSGATSAETNSYFAKSMEFLARPVPAPRTVFRN
AQQQRFDAGA

Accordingly, preferably NSP3 comprises an amino acid sequence as substantially as set out in SEQ ID No: 84, or a biologically active variant or fragment thereof.

In one embodiment, NSP3 is encoded by a nucleotide sequence a defined in SEQ ID No: 85, as follows:

[SEQ ID No: 85]
GCACCCTCATATCATGTGGTGCGAGGGGATATTGCCACGGCCACCGAAGGAGTGATTATAAATGCTGCTAACAGCAAA
GGACAACCTGGCGGAGGGGTGTGCGGAGCGCTGTATAAGAAATTCCCGGAAAGCTTCGATTTACAGCCGATCGAAGTA
GGAAAAGCGCGACTGGTCAAAGGTGCAGCTAAACATATCATTCATGCCGTAGGACCAAACTTCAACAAAGTTTCGGAG
GTTGAAGGTGACAAACAGTTGGCAGAGGCTTATGAGTCCATCGCTAAGATTGTCAACGATAACAATTACAAGTCAGTA
GCGATTCCACTGTTGTCCACCGGCATCTTTTCCGGGAACAAAGATCGACTAACCCAATCATTGAACCATTTGCTGACA
GCTTTAGACACCACTGATGCAGATGTAGCCATATACTGCAGGGACAAGAAATGGGAAATGACTCTCAAGGAAGCAGTG
GCTAGGAGAGAAGCAGTGGAGGAGATATGCATATCCGACGACTCTTCAGTGACAGAACCTGATGCAGAGCTGGTGAGG
GTGCATCCGAAGAGTTCTTTGGCTGGAAGGAAGGGCTACAGCACAAGCGATGGCAAAACTTTCTCATATTTGGAAGGG
ACCAAGTTTCACCAGGCGGCCAAGGATATAGCAGAAATTAATGCCATGTGGCCCGTTGCAACGGAGGCCAATGAGCAG
GTATGCATGTATATCCTCGGAGAAAGCATGAGCAGTATTAGGTCGAAATGCCCCGTCGAAGAGTCGGAAGCCTCCACA
CCACCTAGCACGCTGCCTTGCTTGTGCATCCATGCCATGACTCCAGAAAGAGTACAGCGCCTAAAAGCCTCACGTCCA
GAACAAATTACTGTGTGCTCATCCTTTCCATTGCCGAAGTATAGAATCACTGGTGTGCAGAAGATCCAATGCTCCCAG
CCTATATTGTTCTCACCGAAAGTGCCTGCGTATATTCATCCAAGGAAGTATCTCGTGGAAACACCACCGGTAGACGAG
ACTCCGGAGCCATCGGCAGAGAACCAATCCACAGAGGGGACACCTGAACAACCACCACTTATAACCGAGGATGAGACC
AGGACTAGAACGCCTGAGCCGATCATCATCGAAGAGGAAGAAGAGGATAGCATAAGTTTGCTGTCAGATGGCCCGACC
CACCAGGTGCTGCAAGTCGAGGCAGACATTCACGGGCCGCCCTCTGTATCTAGCTCATCCTGGTCCATTCCTCATGCA
TCCGACTTTGATGTGGACAGTTTATCCATACTTGACACCCTGGAGGGAGCTAGCGTGACCAGCGGGGCAACGTCAGCC
GAGACTAACTCTTACTTCGCAAAGAGTATGGAGTTTCTGGCGCGACCGGTGCCTGCGCCTCGAACAGTATTCAGGAAC
CCTCCACATCCCGCTCCGCGCACAAGAACACCGTCACTTGCACCCAGCAGGGCCTGCTCGAGAACCAGCCTAGTTTCC
ACCCCGCCAGGCGTGAATAGGGTGATCACTAGAGAGGAGCTCGAGGCGCTTACCCCGTCACGCACTCCTAGCAGGTCG
GCACAACAACAATGACGGTTTGATGCGGGTGCA

Accordingly, preferably NSP3 comprises an amino acid sequence as substantially as set out in SEQ ID No: 85, or a biologically active variant or fragment thereof.

Thus, the RNA construct may comprise SEQ ID No: 86, as follows:

[SEQ ID No: 86]
GCACCCUCAUAUCAUGUGGUGCGAGGGGAUAUUGCCACGGCCACCGAAGGAGUGAUUAUAAAUGCUGCUAACAGCAAA
GGACAACCUGGCGGAGGGGUGUGCGGAGCGCUGUAUAAGAAAUUCCCGGAAAGCUUCGAUUUACAGCCGAUCGAAGUA
GGAAAAGCGCGACUGGUCAAAGGUGCAGCUAAACAUAUCAUUCAUGCCGUAGGACCAAACUUCAACAAAGUUUCGGAG
GUUGAAGGUGACAAACAGUUGGCAGAGGCUUAUGAGUCCAUCGCUAAGAUUGUCAACGAUAACAAUUACAAGUCAGUA
GCGAUUCCACUGUUGUCCACCGGCAUCUUUUCCGGGAACAAAGAUCGACUAACCCAAUCAUUGAACCAUUUGCUGACA
GCUUUAGACACCACUGAUGCAGAUGUAGCCAUAUACUGCAGGGACAAGAAAUGGGAAAUGACUCUCAAGGAAGCAGUG
GCUAGGAGAGAAGCAGUGGAGGAGAUAUGCAUAUCCGACGACUCUUCAGUGACAGAACCUGAUGCAGAGCUGGUGAGG
GUGCAUCCGAAGAGUUCUUUGGCUGGAAGGAAGGGCUACAGCACAAGCGAUGGCAAAACUUUCUCAUAUUUGGAAGGG
ACCAAGUUUCACCAGGCGGCCAAGGAUAUAGCAGAAAUUAAUGCCAUGUGGCCCGUUGCAACGGAGGCCAAUGAGCAG
GUAUGCAUGUAUAUCCUCGGAGAAAGCAUGAGCAGUAUUAGGUCGAAAUGCCCCGUCGAAGAGUCGGAAGCCUCCACA
CCACCUAGCACGCUGCCUUGCUUGUGCAUCCAUGCCAUGACUCCAGAAAGAGUACAGCGCCUAAAAGCCUCACGUCCA
GAACAAAUUACUGUGUGCUCAUCCUUUCCAUUGCCGAAGUAUAGAAUCACUGGUGUGCAGAAGAUCCAAUGCUCCCAG
CCUAUAUUGUUCUCACCGAAAGUGCCUGCGUAUAUUCAUCCAAGGAAGUAUCUCGUGGAAACACCACCGGUAGACGAG
ACUCCGGAGCCAUCGGCAGAGAACCAAUCCACAGAGGGGACACCUGAACAACCACCACUUAUAACCGAGGAUGAGACC
AGGACUAGAACGCCUGAGCCGAUCAUCAUCGAAGAGGAAGAAGAGGAUAGCAUAAGUUUGCUGUCAGAUGGCCCGACC
CACCAGGUGCUGCAAGUCGAGGCAGACAUUCACGGGCCGCCCUCUGUAUCUAGCUCAUCCUGGUCCAUUCCUCAUGCA
UCCGACUUUGAUGUGGACAGUUUAUCCAUACUUGACACCCUGGAGGGAGCUAGCGUGACCAGCGGGGCAACGUCAGCC
GAGACUAACUCUUACUUCGCAAAGAGUAUGGAGUUUCUGGCGCGACCGGUGCCUGCGCCUCGAACAGUAUUCAGGAAC
CCUCCACAUCCCGCUCCGCGCACAAGAACACCGUCACUUGCACCCAGCAGGGCCUGCUCGAGAACCAGCCUAGUUUCC
ACCCCGCCAGGCGUGAAUAGGGUGAUCACUAGAGAGGAGCUCGAGGCGCUUACCCCGUCACGCACUCCUAGCAGGUCG
GUCUCGAGAACCAGCCUGGUCUCCAACCCGCCAGGCGUAAAUAGGGUGAUUACAAGAGAGGAGUUUGAGGCGUUCGUA
GCACAACAACAAUGACGGUUUGAUGCGGGUGCA

Accordingly, therefore, preferably the RNA construct comprises an RNA nucleotide sequence substantially as set out as SEQ ID No: 86, or a variant or fragment thereof.

In one embodiment, NSP4 is provided herein as SEQ ID No: 87, as follows:

[SEQ ID No: 87]
YIFSSDTGQGHLQQKSVRQTVLSEVVLERTELEISYAPRLDQEKEELLRKKLQLNPTPANRSRYQSRKVENMKAITAR
RILQGLGHYLKAEGKVECYRTLHPVPLYSSSVNRAFSSPKVAVEACNAMLKENFPTVASYCIIPEYDAYLDMVDGASC
CLDTASFCPAKLRSFPKKHSYLEPTIRSAVPSAIQNTLQNVLAAATKRNCNVTQMRELPVLDSAAFNVECFKKYACNN
EYWETFKENPIRLTEENVVNYITKLKGPKAAALFAKTHNLNMLQDIPMDRFVMDLKRDVKVTPGTKHTEERPKVQVIQ
AADPLATAYLCGIHRELVRRLNAVLLPNIHTLFDMSAEDFDAIIAEHFQPGDCVLETDIASFDKSEDDAMALTALMIL
EDLGVDAELLTLIEAAFGEISSIHLPTKTKFKFGAMMKSGMFLTLFVNTVINIVIASRVLRERLIGSPCAAFIGDDNI
VKGVKSDKLMADRCATWLNMEVKIIDAVVGEKAPYFCGGFILCDSVTGTACRVADPLKRLFKLGKPLAADDEHDDDRR
RALHEESTRWNRVGILSELCKAVESRYETVGTSIIVMAMTTLASSVKSFSYLRGAPITLYG

Accordingly, preferably NSP4 comprises an amino acid sequence as substantially as set out in SEQ ID No: 87, or a biologically active variant or fragment thereof.

In one embodiment, NSP4 is encoded by a nucleotide sequence a defined in SEQ ID No: 88, as follows:

[SEQ ID No: 88]
TACATCTTTTCCTCCGACACCGGTCAAGGGCATTTACAACAAAAATCAGTAAGGCAAACGGTGCTATCCGAAGTGGTG
TTGGAGAGGACCGAATTGGAGATTTCGTATGCCCCGCGCCTCGACCAAGAAAAAGAAGAATTACTACGCAAGAAATTA
CAGTTAAATCCCACACCTGCTAACAGAAGCAGATACCAGTCCAGGAAGGTGGAGAACATGAAAGCCATAACAGCTAGA
CGTATTCTGCAAGGCCTAGGGCATTATTTGAAGGCAGAAGGAAAAGTGGAGTGCTACCGAACCCTGCATCCTGTTCCT
TTGTATTCATCTAGTGTGAACCGTGCCTTTTCAAGCCCCAAGGTCGCAGTGGAAGCCTGTAACGCCATGTTGAAAGAG
AACTTTCCGACTGTGGCTTCTTACTGTATTATTCCAGAGTACGATGCCTATTTGGACATGGTTGACGGAGCTTCATGC
TGCTTAGACACTGCCAGTTTTTGCCCTGCAAAGCTGCGCAGCTTTCCAAAGAAACACTCCTATTTGGAACCCACAATA
CGATCGGCAGTGCCTTCAGCGATCCAGAACACGCTCCAGAACGTCCTGGCAGCTGCCACAAAAAGAAATTGCAATGTC
ACGCAAATGAGAGAATTGCCCGTATTGGATTCGGCGGCCTTTAATGTGGAATGCTTCAAGAAATATGCGTGTAATAAT
GAATATTGGGAAACGTTTAAAGAAAACCCCATCAGGCTTACTGAAGAAAACGTGGTAAATTACATTACCAAATTAAAA
GGACCAAAAGCTGCTGCTCTTTTTGCGAAGACACATAATTTGAATATGTTGCAGGACATACCAATGGACAGGTTTGTA
ATGGACTTAAAGAGAGACGTGAAAGTGACTCCAGGAACAAAACATACTGAAGAACGGCCCAAGGTACAGGTGATCCAG
GCTGCCGATCCGCTAGCAACAGCGTATCTGTGCGGAATCCACCGAGAGCTGGTTAGGAGATTAAATGCGGTCCTGCTT
CCGAACATTCATACACTGTTTGATATGTCGGCTGAAGACTTTGACGCTATTATAGCCGAGCACTTCCAGCCTGGGGAT
TGTGTTCTGGAAACTGACATCGCGTCGTTTGATAAAAGTGAGGACGACGCCATGGCTCTGACCGCGTTAATGATTCTG
GAAGACTTAGGTGTGGACGCAGAGCTGTTGACGCTGATTGAGGCGGCTTTCGGCGAAATTTCATCAATACATTTGCCC
ACTAAAACTAAATTTAAATTCGGAGCCATGATGAAATCTGGAATGTTCCTCACACTGTTTGTGAACACAGTCATTAAC
ATTGTAATCGCAAGCAGAGTGTTGAGAGAACGGCTAACCGGATCACCATGTGCAGCATTCATTGGAGATGACAATATC
GTGAAAGGAGTCAAATCGGACAAATTAATGGCAGACAGGTGCGCCACCTGGTTGAATATGGAAGTCAAGATTATAGAT
GCTGTGGTGGGCGAGAAAGCGCCTTATTTCTGTGGAGGGTTTATTTTGTGTGACTCCGTGACCGGCACAGCGTGCCGT
GTGGCAGACCCCCTAAAAAGGCTGTTTAAGCTTGGCAAACCTCTGGCAGCAGACGATGAACATGATGATGACAGGAGA
AGGGCATTGCATGAAGAGTCAACACGCTGGAACCGAGTGGGTATTCTTTCAGAGCTGTGCAAGGCAGTAGAATCAAGG
TATGAAACCGTAGGAACTTCCATCATAGTTATGGCCATGACTACTCTAGCTAGCAGTGTTAAATCATTCAGCTACCTG
AGAGGGGCCCCTATAACTCTCTACGGC

Accordingly, preferably NSP4 is encoded by a nucleotide sequence as substantially as set out in SEQ ID No: 88, or a variant or fragment thereof.

Thus, the RNA construct may comprise SEQ ID No: 89, as follows:

[SEQ ID No: 89]
UACAUCUUUUCCUCCGACACCGGUCAAGGGCAUUUACAACAAAAAUCAGUAAGGCAAACGGUGCUAUCCGAAGUGGUG
UUGGAGAGGACCGAAUUGGAGAUUUCGUAUGCCCCGCGCCUCGACCAAGAAAAAGAAGAAUUACUACGCAAGAAAUUA
CAGUUAAAUCCCACACCUGCUAACAGAAGCAGAUACCAGUCCAGGAAGGUGGAGAACAUGAAAGCCAUAACAGCUAGA
CGUAUUCUGCAAGGCCUAGGGCAUUAUUUGAAGGCAGAAGGAAAAGUGGAGUGCUACCGAACCCUGCAUCCUGUUCCU
UUGUAUUCAUCUAGUGUGAACCGUGCCUUUUCAAGCCCCAAGGUCGCAGUGGAAGCCUGUAACGCCAUGUUGAAAGAG
AACUUUCCGACUGUGGCUUCUUACUGUAUUAUUCCAGAGUACGAUGCCUAUUUGGACAUGGUUGACGGAGCUUCAUGC
UGCUUAGACACUGCCAGUUUUUGCCCUGCAAAGCUGCGCAGCUUUCCAAAGAAACACUCCUAUUUGGAACCCACAAUA
CGAUCGGCAGUGCCUUCAGCGAUCCAGAACACGCUCCAGAACGUCCUGGCAGCUGCCACAAAAAGAAAUUGCAAUGUC
ACGCAAAUGAGAGAAUUGCCCGUAUUGGAUUCGGCGGCCUUUAAUGUGGAAUGCUUCAAGAAAUAUGCGUGUAAUAAU
GAAUAUUGGGAAACGUUUAAAGAAAACCCCAUCAGGCUUACUGAAGAAAACGUGGUAAAUUACAUUACCAAAUUAAAA
GGACCAAAAGCUGCUGCUCUUUUUGCGAAGACACAUAAUUUGAAUAUGUUGCAGGACAUACCAAUGGACAGGUUUGUA
AUGGACUUAAAGAGAGACGUGAAAGUGACUCCAGGAACAAAACAUACUGAAGAACGGCCCAAGGUACAGGUGAUCCAG
GCUGCCGAUCCGCUAGCAACAGCGUAUCUGUGCGGAAUCCACCGAGAGCUGGUUAGGAGAUUAAAUGCGGUCCUGCUU
CCGAACAUUCAUACACUGUUUGAUAUGUCGGCUGAAGACUUUGACGCUAUUAUAGCCGAGCACUUCCAGCCUGGGGAU
UGUGUUCUGGAAACUGACAUCGCGUCGUUUGAUAAAAGUGAGGACGACGCCAUGGCUCUGACCGCGUUAAUGAUUCUG
GAAGACUUAGGUGUGGACGCAGAGCUGUUGACGCUGAUUGAGGCGGCUUUCGGCGAAAUUUCAUCAAUACAUUUGCCC
ACUAAAACUAAAUUUAAAUUCGGAGCCAUGAUGAAAUCUGGAAUGUUCCUCACACUGUUUGUGAACACAGUCAUUAAC
AUUGUAAUCGCAAGCAGAGUGUUGAGAGAACGGCUAACCGGAUCACCAUGUGCAGCAUUCAUUGGAGAUGACAAUAUC
GUGAAAGGAGUCAAAUCGGACAAAUUAAUGGCAGACAGGUGCGCCACCUGGUUGAAUAUGGAAGUCAAGAUUAUAGAU
GCUGUGGUGGGCGAGAAAGCGCCUUAUUUCUGUGGAGGGUUUAUUUUGUGUGACUCCGUGACCGGCACAGCGUGCCGU
GUGGCAGACCCCCUAAAAAGGCUGUUUAAGCUUGGCAAACCUCUGGCAGCAGACGAUGAACAUGAUGAUGACAGGAGA
AGGGCAUUGCAUGAAGAGUCAACACGCUGGAACCGAGUGGGUAUUCUUUCAGAGCUGUGCAAGGCAGUAGAAUCAAGG
UAUGAAACCGUAGGAACUUCCAUCAUAGUUAUGGCCAUGACUACUCUAGCUAGCAGUGUUAAAUCAUUCAGCUACCUG
AGAGGGGCCCCUAUAACUCUCUACGGC

Accordingly, therefore, preferably the RNA construct comprises an RNA nucleotide sequence substantially as set out as SEQ ID No: 89, or a variant or fragment thereof.

Preferably, together with proteins present in a host cell, the non-structural proteins encoded by the RNA construct of the invention form an enzyme complex (i.e. a replicase) that is required for genome replication and transcription of the sequences encoding the at least one therapeutic biomolecule and the at least one innate modulatory protein. For example, the one or more non-structural protein may encode a polymerase to enable the construct to amplify the nucleotide sequences encoding the at least one peptide or protein of interest (i.e. therapeutic biomolecule) and the at least one innate modulatory protein.

The host cell may be a eukaryotic or prokaryotic host cell. Preferably, the host cell is a eukaryotic host cell. More preferably, the host cell is a mammalian host cell.

The RNA construct may further comprise a promoter disposed 5′ of the at least one non-structural protein, such that the promoter is operably linked to the sequence encoding the at least one non-structural protein and enables expression of the at least one non-structural protein in a host cell.

Preferably, the RNA construct comprises a 5′ UTR conserved sequence element, which may be referred to herein as SEQ ID No: 90, as follows:

[SEQ ID No: 90]
AUGGGCGGCGCAUGAGAGAAGCCCAGACCAAUUACCUACCCAAA

Accordingly, preferably the UTR is disposed 5′ of the at least one non-structural protein and comprises a nucleotide sequence substantially as set out in SEQ ID No: 90, or a fragment or variant thereof.

Preferably, the RNA construct comprises a 3′ UTR conserved sequence element, which may be referred to herein as SEQ ID No: 91, as follows:

[SEQ ID No: 91]
AAUUGGCAAGCUGCUUACAUAGAACUCGCGGCGAUUGGCAUGCCGCCUU
AAAAUUUUUAUUUUAUUUUUCUUUUCUUUUCCGAAUCGGAUUUUGUUUU
UAAUAUUUCAAAAAAAAAAAAAAAAAAAAAAAAAAAAAAAAAAA

Accordingly, preferably the 3′ UTR is disposed 3′ of the at least one non-structural protein and comprises a nucleotide sequence substantially as set out in SEQ ID No: 91, or a fragment or variant thereof.

Preferably, the RNA construct comprises a polyA tail. Preferably, the polyA tail is disposed at the 3′ end of the construct. The poly A tail may comprise at least 35 nt, or at least 40 nt, or at least 45 nt, or at least 50 nt, wherein each nt is an adenine. In another embodiment, the polyA tail may comprise at least 55 nt or at least 60 nt, wherein each nt is an adenine. In yet another embodiment, the polyA tail may comprise at least 60 adenines, followed by one or more non-adenine nucleotides (i.e. G, C or T, preferably guanine), and then another at least 35 nt, or at least 40 nt, or at least 45 nt, or at least 50 nt, or at least 55 nt, or at least 60 nt, wherein each nt is an adenine.

The RNA construct may further comprise a 5′ cap. In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

An RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA construct comprises, preferably 5′ to 3′, a promoter, a sequence encoding at least one therapeutic biomolecule, a linker sequence, and at least one sequence encoding an IIP. In one embodiment, the RNA construct comprises, preferably 5′ to 3′, a promoter, a sequence encoding at least one IIP, a linker sequence, and a sequence encoding at least one therapeutic biomolecule. The linker may be F-T2a or IRES in either embodiment.

In another embodiment, the RNA construct comprises, preferably 5′ to 3′, a promoter, a sequence encoding at least one non-structural protein, a sub genomic promoter, a sequence encoding at least one therapeutic biomolecule, a linker sequence, and a sequence encoding at least one viral IIP. In another embodiment, the RNA construct comprises, preferably 5′ to 3′, a promoter, a sequence encoding at least one non-structural protein, a sub genomic promoter, a sequence encoding at least one viral IIP, a linker sequence, and a sequence encoding at least one therapeutic biomolecule. The linker may be F-T2a or IRES in either embodiment.

In yet another embodiment, the RNA construct comprises, preferably 5′ to 3′, a promoter, a sequence encoding at least one non-structural protein, a sub genomic promoter, a sequence encoding at least one therapeutic biomolecule, a linker sequence, a sequence encoding at least one viral IIP, and a polyA tail. In yet another embodiment, the RNA construct comprises, preferably 5′ to 3′, a promoter, a sequence encoding at least one non-structural protein, a sub genomic promoter, a sequence encoding at least one viral IIP, a linker sequence, a sequence encoding at least one therapeutic biomolecule, and a polyA tail. The linker may be F-T2a or IRES in either embodiment.

In another embodiment, the RNA construct comprises, preferably 5′ to 3′, a promoter, a sequence encoding at least one non-structural protein, a first sub genomic promoter, a sequence encoding at least one therapeutic biomolecule, a second sub genomic promoter, a sequence encoding at least one viral IIP, and a polyA tail. In another embodiment, the RNA construct comprises, preferably 5′ to 3′, a promoter, a sequence encoding at least one non-structural protein, a first sub genomic promoter, a sequence encoding at least one viral IIP, a second sub genomic promoter, a sequence encoding at least one therapeutic biomolecule, and a polyA tail.

Most preferably, the RNA construct comprises, 5′ to 3′, a 5′ cap, a promoter, nsP1, nsP2, nsP3, nsP4, the sub genomic promoter 26S, a sequence encoding a therapeutic biomolecule, a linker sequence, a sequence encoding the viral IIP and a polyA tail. Most preferably, the RNA construct comprises, 5′ to 3′, a 5′ cap, a promoter, nsP1, nsP2, nsP3v, nsP4, the sub genomic promoter 26S, a sequence encoding a viral IIP, a linker sequence, a sequence encoding a therapeutic biomolecule; and a polyA tail.

In one embodiment, therefore, the RNA construct may encode or comprise a GOI—furin T2A—HCV E6 (which is the first viral IIP mentioned herein, but it will be appreciated that any of the IIPs or linkers disclosed herein may be used). Hence, the RNA construct may comprise or consist of a single RNA construct comprising or consisting of SEQ ID No: 92, a GOI, and SEQ ID No: 457, in a single RNA construct. SEQ ID No: 92 and SEQ ID No: 457 are as follows:

[SEQ ID No: 92]
AUGGGCGGCGCAUGAGAGAAGCCCAGACCAAUUACCUACCCAAAAUGGAGAAAGUUCACGUUGACAUCGAGG
AAGACAGCCCAUUCCUCAGAGCUUUGCAGCGGAGCUUCCCGCAGUUUGAGGUAGAAGCCAAGCAGGUCACUG
AUAAUGACCAUGCUAAUGCCAGAGCGUUUUCGCAUCUGGCUUCAAAACUGAUCGAAACGGAGGUGGACCCAU
CCGACACGAUCCUUGACAUUGGAAGUGCGCCCGCCCGCAGAAUGUAUUCUAAGCACAAGUAUCAUUGUAUCU
GUCCGAUGAGAUGUGCGGAAGAUCCGGACAGAUUGUAUAAGUAUGCAACUAAGCUGAAGAAAAACUGUAAGG
AAAUAACUGAUAAGGAAUUGGACAAGAAAAUGAAGGAGCUGGCCGCCGUCAUGAGCGACCCUGACCUGGAAA
CUGAGACUAUGUGCCUCCACGACGACGAGUCGUGUCGCUACGAAGGGCAAGUCGCUGUUUACCAGGAUGUAU
ACGCGGUUGACGGACCGACAAGUCUCUAUCACCAAGCCAAUAAGGGAGUUAGAGUCGCCUACUGGAUAGGCU
UUGACACCACCCCUUUUAUGUUUAAGAACUUGGCUGGAGCAUAUCCAUCAUACUCUACCAACUGGGCCGACG
AAACCGUGUUAACGGCUCGUAACAUAGGCCUAUGCAGCUCUGACGUUAUGGAGCGGUCACGUAGAGGGAUGU
CCAUUCUUAGAAAGAAGUAUUUGAAACCAUCCAACAAUGUUCUAUUCUCUGUUGGCUCGACCAUCUACCACG
AGAAGAGGGACUUACUGAGGAGCUGGCACCUGCCGUCUGUAUUUCACUUACGUGGCAAGCAAAAUUACACAU
GUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGA
ACGGGGAGAGGGUCUCUUUUCCCGUGUGCACGUAUGUGCCAGCUACAUUGUGUGACCAAAUGACUGGCAUAC
UGGCAACAGAUGUCAGUGCGGACGACGCGCAAAAACUGCUGGUUGGGCUCAACCAGCGUAUAGUCGUCAACG
GUCGCACCCAGAGAAACACCAAUACCAUGAAAAAUUACCUUUUGCCCGUAGUGGCCCAGGCAUUUGCUAGGU
GGGCAAAGGAAUAUAAGGAAGAUCAAGAAGAUGAAAGGCCACUAGGACUACGAGAUAGACAGUUAGUCAUGG
GGUGUUGUUGGGCUUUUAGAAGGCACAAGAUAACAUCUAUUUAUAAGCGCCCGGAUACCCAAACCAUCAUCA
AAGUGAACAGCGAUUUCCACUCAUUCGUGCUGCCCAGGAUAGGCAGUAACACAUUGGAGAUCGGGCUGAGAA
CAAGAAUCAGGAAAAUGUUAGAGGAGCACAAGGAGCCGUCACCUCUCAUUACCGCCGAGGACGUACAAGAAG
CUAAGUGCGCAGCCGAUGAGGCUAAGGAGGUGCGUGAAGCCGAGGAGUUGCGCGCAGCUCUACCACCUUUGG
CAGCUGAUGUUGAGGAGCCCACUCUGGAAGCCGAUGUCGACUUGAUGUUACAAGAGGCUGGGGCCGGCUCAG
UGGAGACACCUCGUGGCUUGAUAAAGGUUACCAGCUACGAUGGCGAGGACAAGAUCGGCUCUUACGCUGUGC
UUUCUCCGCAGGCUGUACUCAAGAGUGAAAAAUUAUCUUGCAUCCACCCUCUCGCUGAACAAGUCAUAGUGA
UAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCG
UAAACAGGUACCUGCACCAUAUUGCCACACAUGGAGGAGCGCUGAACACUGAUGAAGAAUAUUACAAAACUG
UCAAGCCCAGCGAGCACGACGGCGAAUACCUGUACGACAUCGACAGGAAACAGUGCGUCAAGAAAGAACUAG
UCACUGGGCUAGGGCUCACAGGCGAGCUGGUGGAUCCUCCCUUCCAUGAAUUCGCCUACGAGAGUCUGAGAA
CACGACCAGCCGCUCCUUACCAAGUACCAACCAUAGGGGUGUAUGGCGUGCCAGGAUCAGGCAAGUCUGGCA
UCAUUAAAAGCGCAGUCACCAAAAAAGAUCUAGUGGUGAGCGCCAAGAAAGAAAACUGUGCAGAAAUUAUAA
GGGACGUCAAGAAAAUGAAAGGGCUGGACGUCAAUGCCAGAACUGUGGACUCAGUGCUCUUGAAUGGAUGCA
AACACCCCGUAGAGACCCUGUAUAUUGACGAAGCUUUUGCUUGUCAUGCAGGUACUCUCAGAGCGCUCAUAG
CCAUUAUAAGACCUAAAAAGGCAGUGCUCUGCGGGGAUCCCAAACAGUGCGGUUUUUUUAACAUGAUGUGCC
UGAAAGUGCAUUUUAACCACGAGAUUUGCACACAAGUCUUCCACAAAAGCAUCUCUCGCCGUUGCACUAAAU
CUGUGACUUCGGUCGUCUCAACCUUGUUUUACGACAAAAAAAUGAGAACGACGAAUCCGAAAGAGACUAAGA
UUGUGAUUGACACUACCGGCAGUACCAAACCUAAGCAGGACGAUCUCAUUCUCACUUGUUUCAGAGGGUGGG
UGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACG
UCCUACUGACCCGCACGGAGGACCGCAUCGUGUGGAAAACACUAGCCGGCGACCCAUGGAUAAAAACACUGA
CUGCCAAGUACCCUGGGAAUUUCACUGCCACGAUAGAGGAGUGGCAAGCAGAGCAUGAUGCCAUCAUGAGGC
ACAUCUUGGAGAGACCGGACCCUACCGACGUCUUCCAGAAUAAGGCAAACGUGUGUUGGGCCAAGGCUUUAG
UGCCGGUGCUGAAGACCGCUGGCAUAGACAUGACCACUGAACAAUGGAACACUGUGGAUUAUUUUGAAACGG
ACAAAGCUCACUCAGCAGAGAUAGUAUUGAACCAACUAUGCGUGAGGUUCUUUGGACUCGAUCUGGACUCCG
GUCUAUUUUCUGCACCCACUGUUCCGUUAUCCAUUAGGAAUAAUCACUGGGAUAACUCCCCGUCGCCUAACA
UGUACGGGCUGAAUAAAGAAGUGGUCCGUCAGCUCUCUCGCAGGUACCCACAACUGCCUCGGGCAGUUGCCA
CUGGAAGAGUCUAUGACAUGAACACUGGUACACUGCGCAAUUAUGAUCCGCGCAUAAACCUAGUACCUGUAA
ACAGAAGACUGCCUCAUGCUUUAGUCCUCCACCAUAAUGAACACCCACAGAGUGACUUUUCUUCAUUCGUCA
GCAAAUUGAAGGGCAGAACUGUCCUGGUGGUCGGGGAAAAGUUGUCCGUCCCAGGCAAAAUGGUUGACUGGU
UGUCAGACCGGCCUGAGGCUACCUUCAGAGCUCGGCUGGAUUUAGGCAUCCCAGGUGAUGUGCCCAAAUAUG
ACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUG
GUUACGCUGACAGGGCCAGCGAAAGCAUCAUUGGUGCUAUAGCGCGGCAGUUCAAGUUUUCCCGGGUAUGCA
AACCGAAAUCCUCACUUGAAGAGACGGAAGUUCUGUUUGUAUUCAUUGGGUACGAUCGCAAGGCCCGUACGC
ACAAUUCUUACAAGCUUUCAUCAACCUUGACCAACAUUUAUACAGGUUCCAGACUCCACGAAGCCGGAUGUG
CACCCUCAUAUCAUGUGGUGCGAGGGGAUAUUGCCACGGCCACCGAAGGAGUGAUUAUAAAUGCUGCUAACA
GCAAAGGACAACCUGGCGGAGGGGUGUGCGGAGCGCUGUAUAAGAAAUUCCCGGAAAGCUUCGAUUUACAGC
CGAUCGAAGUAGGAAAAGCGCGACUGGUCAAAGGUGCAGCUAAACAUAUCAUUCAUGCCGUAGGACCAAACU
UCAACAAAGUUUCGGAGGUUGAAGGUGACAAACAGUUGGCAGAGGCUUAUGAGUCCAUCGCUAAGAUUGUCA
ACGAUAACAAUUACAAGUCAGUAGCGAUUCCACUGUUGUCCACCGGCAUCUUUUCCGGGAACAAAGAUCGAC
UAACCCAAUCAUUGAACCAUUUGCUGACAGCUUUAGACACCACUGAUGCAGAUGUAGCCAUAUACUGCAGGG
ACAAGAAAUGGGAAAUGACUCUCAAGGAAGCAGUGGCUAGGAGAGAAGCAGUGGAGGAGAUAUGCAUAUCCG
ACGACUCUUCAGUGACAGAACCUGAUGCAGAGCUGGUGAGGGUGCAUCCGAAGAGUUCUUUGGCUGGAAGGA
AGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCG
GAGAAAGCAUGAGCAGUAUUAGGUCGAAAUGCCCCGUCGAAGAGUCGGAAGCCUCCACACCACCUAGCACGC
UGCCUUGCUUGUGCAUCCAUGCCAUGACUCCAGAAAGAGUACAGCGCCUAAAAGCCUCACGUCCAGAACAAA
UUACUGUGUGCUCAUCCUUUCCAUUGCCGAAGUAUAGAAUCACUGGUGUGCAGAAGAUCCAAUGCUCCCAGC
CUAUAUUGUUCUCACCGAAAGUGCCUGCGUAUAUUCAUCCAAGGAAGUAUCUCGUGGAAACACCACCGGUAG
ACGAGACUCCGGAGCCAUCGGCAGAGAACCAAUCCACAGAGGGGACACCUGAACAACCACCACUUAUAACCG
AGGAUGAGACCAGGACUAGAACGCCUGAGCCGAUCAUCAUCGAAGAGGAAGAAGAGGAUAGCAUAAGUUUGC
UGUCAGAUGGCCCGACCCACCAGGUGCUGCAAGUCGAGGCAGACAUUCACGGGCCGCCCUCUGUAUCUAGCU
CAUCCUGGUCCAUUCCUCAUGCAUCCGACUUUGAUGUGGACAGUUUAUCCAUACUUGACACCCUGGAGGGAG
CUAGCGUGACCAGCGGGGCAACGUCAGCCGAGACUAACUCUUACUUCGCAAAGAGUAUGGAGUUUCUGGCGC
GACCGGUGCCUGCGCCUCGAACAGUAUUCAGGAACCCUCCACAUCCCGCUCCGCGCACAAGAACACCGUCAC
UUGCACCCAGCAGGGCCUGCUCGAGAACCAGCCUAGUUUCCACCCCGCCAGGCGUGAAUAGGGUGAUCACUA
GAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGU
UUGAUGCGGGUGCAUACAUCUUUUCCUCCGACACCGGUCAAGGGCAUUUACAACAAAAAUCAGUAAGGCAAA
CGGUGCUAUCCGAAGUGGUGUUGGAGAGGACCGAAUUGGAGAUUUCGUAUGCCCCGCGCCUCGACCAAGAAA
AAGAAGAAUUACUACGCAAGAAAUUACAGUUAAAUCCCACACCUGCUAACAGAAGCAGAUACCAGUCCAGGA
AGGUGGAGAACAUGAAAGCCAUAACAGCUAGACGUAUUCUGCAAGGCCUAGGGCAUUAUUUGAAGGCAGAAG
GAAAAGUGGAGUGCUACCGAACCCUGCAUCCUGUUCCUUUGUAUUCAUCUAGUGUGAACCGUGCCUUUUCAA
GCCCCAAGGUCGCAGUGGAAGCCUGUAACGCCAUGUUGAAAGAGAACUUUCCGACUGUGGCUUCUUACUGUA
UUAUUCCAGAGUACGAUGCCUAUUUGGACAUGGUUGACGGAGCUUCAUGCUGCUUAGACACUGCCAGUUUUU
GCCCUGCAAAGCUGCGCAGCUUUCCAAAGAAACACUCCUAUUUGGAACCCACAAUACGAUCGGCAGUGCCUU
CAGCGAUCCAGAACACGCUCCAGAACGUCCUGGCAGCUGCCACAAAAAGAAAUUGCAAUGUCACGCAAAUGA
GAGAAUUGCCCGUAUUGGAUUCGGCGGCCUUUAAUGUGGAAUGCUUCAAGAAAUAUGCGUGUAAUAAUGAAU
AUUGGGAAACGUUUAAAGAAAACCCCAUCAGGCUUACUGAAGAAAACGUGGUAAAUUACAUUACCAAAUUAA
AAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGG
UACAGGUGAUCCAGGCUGCCGAUCCGCUAGCAACAGCGUAUCUGUGCGGAAUCCACCGAGAGCUGGUUAGGA
GAUUAAAUGCGGUCCUGCUUCCGAACAUUCAUACACUGUUUGAUAUGUCGGCUGAAGACUUUGACGCUAUUA
UAGCCGAGCACUUCCAGCCUGGGGAUUGUGUUCUGGAAACUGACAUCGCGUCGUUUGAUAAAAGUGAGGACG
ACGCCAUGGCUCUGACCGCGUUAAUGAUUCUGGAAGACUUAGGUGUGGACGCAGAGCUGUUGACGCUGAUUG
AGGCGGCUUUCGGCGAAAUUUCAUCAAUACAUUUGCCCACUAAAACUAAAUUUAAAUUCGGAGCCAUGAUGA
AAUCUGGAAUGUUCCUCACACUGUUUGUGAACACAGUCAUUAACAUUGUAAUCGCAAGCAGAGUGUUGAGAG
AACGGCUAACCGGAUCACCAUGUGCAGCAUUCAUUGGAGAUGACAAUAUCGUGAAAGGAGUCAAAUCGGACA
AAUUAAUGGCAGACAGGUGCGCCACCUGGUUGAAUAUGGAAGUCAAGAUUAUAGAUGCUGUGGUGGGCGAGA
AAGCGCCUUAUUUCUGUGGAGGGUUUAUUUUGUGUGACUCCGUGACCGGCACAGCGUGCCGUGUGGCAGACC
CCCUAAAAAGGCUGUUUAAGCUUGGCAAACCUCUGGCAGCAGACGAUGAACAUGAUGAUGACAGGAGAAGGG
CAUUGCAUGAAGAGUCAACACGCUGGAACCGAGUGGGUAUUCUUUCAGAGCUGUGCAAGGCAGUAGAAUCAA
GGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCUAACCUGAAUGGACUACGACAUAGUCUAGUCCGCCAA
GUCUAGCAUAUGGCCACC -----------------GOI-----------------
[SEQ ID No: 457]
CGGAGACGGCGCAGAAGAAGAGGAUCUGGCGAAGGCAGAGGCAGCCUGCUUACAUGUGGCGACGUGGAAGAG
AACCCCGGACCUAUGCACCAGAAACGGACCGCCAUGUUCCAGGAUCCUCAAGAGAGGCCCAGAAAGCUGCCU
CAGCUGUGUACCGAGCUGCAGACCACCAUCCACGACAUCAUCCUGGAAUGCGUGUACUGCAAGCAGCAGCUC
CUGCGGAGAGAGGUGUACGAUUUCGCCUUCCGGGACCUGUGCAUCGUGUACAGAGAUGGCAACCCCUACGCC
GUGUGCGACAAGUGCCUGAAGUUCUACAGCAAGAUCAGCGAGUACCGGCACUACUGCUACAGCCUGUACGGC
ACCACACUGGAACAGCAGUACAACAAGCCCCUGUGCGACCUGCUGAUCCGGUGCAUCAACUGCCAGAAACCU
CUGUGCCCCGAGGAAAAGCAGCGGCACCUGGACAAGAAGCAGCGGUUCCACAACAUCAGAGGCCGGUGGACC
GGCAGAUGCAUGAGCUGUUGUCGGAGCAGCAGAACCAGACGGGAAACCCAGCUGUGAGCGGCCGCGAAUUGG
CAAGCUGCUUACAUAGAACUCGCGGCGAUUGGCAUGCCGCCUUAAAAUUUUUAUUUUAUUUUUCUUUUCUUU
UCCGAAUCGGAUUUUGUUUUUAAUAUUUCAAAAAAAAAAAAAAAAAAAAAAAAAAAAAAAAAAA

Accordingly, preferably the RNA construct comprises a nucleotide sequence substantially as set out above, comprising or consisting of SEQ ID No: 92, a GOI, and SEQ ID No: 457, or a fragment or variant thereof.

In a second aspect of the invention, there is provided a nucleic acid sequence encoding the RNA construct of the first aspect.

In one embodiment, the nucleic acid sequence may encode a GOI—furin T2A—HCV E6 (which is the first viral IIP mentioned herein, but it will be appreciated that any of the IIPs or linkers disclosed herein may be used). Thus, the nucleic acid sequence may comprise or consist of SEQ ID No: 93, a GOI, and SEQ ID No: 458. SEQ ID No: 93 and SEQ ID No: 458 are as follows:

[SEQ ID No: 93]
ATGGGCGGCGCATGAGAGAAGCCCAGACCAATTACCTACCCAAAATGGAGAAAGTTCACGTTGACATCGAGG
AAGACAGCCCATTCCTCAGAGCTTTGCAGCGGAGCTTCCCGCAGTTTGAGGTAGAAGCCAAGCAGGTCACTG
ATAATGACCATGCTAATGCCAGAGCGTTTTCGCATCTGGCTTCAAAACTGATCGAAACGGAGGTGGACCCAT
CCGACACGATCCTTGACATTGGAAGTGCGCCCGCCCGCAGAATGTATTCTAAGCACAAGTATCATTGTATCT
GTCCGATGAGATGTGCGGAAGATCCGGACAGATTGTATAAGTATGCAACTAAGCTGAAGAAAAACTGTAAGG
AAATAACTGATAAGGAATTGGACAAGAAAATGAAGGAGCTGGCCGCCGTCATGAGCGACCCTGACCTGGAAA
CTGAGACTATGTGCCTCCACGACGACGAGTCGTGTCGCTACGAAGGGCAAGTCGCTGTTTACCAGGATGTAT
ACGCGGTTGACGGACCGACAAGTCTCTATCACCAAGCCAATAAGGGAGTTAGAGTCGCCTACTGGATAGGCT
TTGACACCACCCCTTTTATGTTTAAGAACTTGGCTGGAGCATATCCATCATACTCTACCAACTGGGCCGACG
AAACCGTGTTAACGGCTCGTAACATAGGCCTATGCAGCTCTGACGTTATGGAGCGGTCACGTAGAGGGATGT
CCATTCTTAGAAAGAAGTATTTGAAACCATCCAACAATGTTCTATTCTCTGTTGGCTCGACCATCTACCACG
AGAAGAGGGACTTACTGAGGAGCTGGCACCTGCCGTCTGTATTTCACTTACGTGGCAAGCAAAATTACACAT
GTCGGTGTGAGACTATAGTTAGTTGCGACGGGTACGTCGTTAAAAGAATAGCTATCAGTCCAGGCCTGTATG
GGAAGCCTTCAGGCTATGCTGCTACGATGCACCGCGAGGGATTCTTGTGCTGCAAAGTGACAGACACATTGA
ACGGGGAGAGGGTCTCTTTTCCCGTGTGCACGTATGTGCCAGCTACATTGTGTGACCAAATGACTGGCATAC
TGGCAACAGATGTCAGTGCGGACGACGCGCAAAAACTGCTGGTTGGGCTCAACCAGCGTATAGTCGTCAACG
GTCGCACCCAGAGAAACACCAATACCATGAAAAATTACCTTTTGCCCGTAGTGGCCCAGGCATTTGCTAGGT
GGGCAAAGGAATATAAGGAAGATCAAGAAGATGAAAGGCCACTAGGACTACGAGATAGACAGTTAGTCATGG
GGTGTTGTTGGGCTTTTAGAAGGCACAAGATAACATCTATTTATAAGCGCCCGGATACCCAAACCATCATCA
AAGTGAACAGCGATTTCCACTCATTCGTGCTGCCCAGGATAGGCAGTAACACATTGGAGATCGGGCTGAGAA
CAAGAATCAGGAAAATGTTAGAGGAGCACAAGGAGCCGTCACCTCTCATTACCGCCGAGGACGTACAAGAAG
CTAAGTGCGCAGCCGATGAGGCTAAGGAGGTGCGTGAAGCCGAGGAGTTGCGCGCAGCTCTACCACCTTTGG
CAGCTGATGTTGAGGAGCCCACTCTGGAAGCCGATGTCGACTTGATGTTACAAGAGGCTGGGGCCGGCTCAG
TGGAGACACCTCGTGGCTTGATAAAGGTTACCAGCTACGATGGCGAGGACAAGATCGGCTCTTACGCTGTGC
TTTCTCCGCAGGCTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGGTGCCAGAGGGAC
ATGCAATACCCGTCCAGGACTTTCAAGCTCTGAGTGAAAGTGCCACCATTGTGTACAACGAACGTGAGTTCG
TAAACAGGTACCTGCACCATATTGCCACACATGGAGGAGCGCTGAACACTGATGAAGAATATTACAAAACTG
TCAAGCCCAGCGAGCACGACGGCGAATACCTGTACGACATCGACAGGAAACAGTGCGTCAAGAAAGAACTAG
TCACTGGGCTAGGGCTCACAGGCGAGCTGGTGGATCCTCCCTTCCATGAATTCGCCTACGAGAGTCTGAGAA
CACGACCAGCCGCTCCTTACCAAGTACCAACCATAGGGGTGTATGGCGTGCCAGGATCAGGCAAGTCTGGCA
TCATTAAAAGCGCAGTCACCAAAAAAGATCTAGTGGTGAGCGCCAAGAAAGAAAACTGTGCAGAAATTATAA
GGGACGTCAAGAAAATGAAAGGGCTGGACGTCAATGCCAGAACTGTGGACTCAGTGCTCTTGAATGGATGCA
AACACCCCGTAGAGACCCTGTATATTGACGAAGCTTTTGCTTGTCATGCAGGTACTCTCAGAGCGCTCATAG
CCATTATAAGACCTAAAAAGGCAGTGCTCTGCGGGGATCCCAAACAGTGCGGTTTTTTTAACATGATGTGCC
TGAAAGTGCATTTTAACCACGAGATTTGCACACAAGTCTTCCACAAAAGCATCTCTCGCCGTTGCACTAAAT
CTGTGACTTCGGTCGTCTCAACCTTGTTTTACGACAAAAAAATGAGAACGACGAATCCGAAAGAGACTAAGA
TTGTGATTGACACTACCGGCAGTACCAAACCTAAGCAGGACGATCTCATTCTCACTTGTTTCAGAGGGTGGG
TGAAGCAGTTGCAAATAGATTACAAAGGCAACGAAATAATGACGGCAGCTGCCTCTCAAGGGCTGACCCGTA
AAGGTGTGTATGCCGTTCGGTACAAGGTGAATGAAAATCCTCTGTACGCACCCACCTCAGAACATGTGAACG
TCCTACTGACCCGCACGGAGGACCGCATCGTGTGGAAAACACTAGCCGGCGACCCATGGATAAAAACACTGA
CTGCCAAGTACCCTGGGAATTTCACTGCCACGATAGAGGAGTGGCAAGCAGAGCATGATGCCATCATGAGGC
ACATCTTGGAGAGACCGGACCCTACCGACGTCTTCCAGAATAAGGCAAACGTGTGTTGGGCCAAGGCTTTAG
TGCCGGTGCTGAAGACCGCTGGCATAGACATGACCACTGAACAATGGAACACTGTGGATTATTTTGAAACGG
ACAAAGCTCACTCAGCAGAGATAGTATTGAACCAACTATGCGTGAGGTTCTTTGGACTCGATCTGGACTCCG
GTCTATTTTCTGCACCCACTGTTCCGTTATCCATTAGGAATAATCACTGGGATAACTCCCCGTCGCCTAACA
TGTACGGGCTGAATAAAGAAGTGGTCCGTCAGCTCTCTCGCAGGTACCCACAACTGCCTCGGGCAGTTGCCA
CTGGAAGAGTCTATGACATGAACACTGGTACACTGCGCAATTATGATCCGCGCATAAACCTAGTACCTGTAA
ACAGAAGACTGCCTCATGCTTTAGTCCTCCACCATAATGAACACCCACAGAGTGACTTTTCTTCATTCGTCA
GCAAATTGAAGGGCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATGTGCCCAAATATG
ACATAATATTTGTTAATGTGAGGACCCCATATAAATACCATCACTATCAGCAGTGTGAAGACCATGCCATTA
AGCTTAGCATGTTGACCAAGAAAGCTTGTCTGCATCTGAATCCCGGCGGAACCTGTGTCAGCATAGGTTATG
GTTACGCTGACAGGGCCAGCGAAAGCATCATTGGTGCTATAGCGCGGCAGTTCAAGTTTTCCCGGGTATGCA
AACCGAAATCCTCACTTGAAGAGACGGAAGTTCTGTTTGTATTCATTGGGTACGATCGCAAGGCCCGTACGC
ACAATTCTTACAAGCTTTCATCAACCTTGACCAACATTTATACAGGTTCCAGACTCCACGAAGCCGGATGTG
CACCCTCATATCATGTGGTGCGAGGGGATATTGCCACGGCCACCGAAGGAGTGATTATAAATGCTGCTAACA
GCAAAGGACAACCTGGCGGAGGGGTGTGCGGAGCGCTGTATAAGAAATTCCCGGAAAGCTTCGATTTACAGC
CGATCGAAGTAGGAAAAGCGCGACTGGTCAAAGGTGCAGCTAAACATATCATTCATGCCGTAGGACCAAACT
TCAACAAAGTTTCGGAGGTTGAAGGTGACAAACAGTTGGCAGAGGCTTATGAGTCCATCGCTAAGATTGTCA
ACGATAACAATTACAAGTCAGTAGCGATTCCACTGTTGTCCACCGGCATCTTTTCCGGGAACAAAGATCGAC
TAACCCAATCATTGAACCATTTGCTGACAGCTTTAGACACCACTGATGCAGATGTAGCCATATACTGCAGGG
ACAAGAAATGGGAAATGACTCTCAAGGAAGCAGTGGCTAGGAGAGAAGCAGTGGAGGAGATATGCATATCCG
ACGACTCTTCAGTGACAGAACCTGATGCAGAGCTGGTGAGGGTGCATCCGAAGAGTTCTTTGGCTGGAAGGA
AGGGCTACAGCACAAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGCATGTATATCCTCG
GAGAAAGCATGAGCAGTATTAGGTCGAAATGCCCCGTCGAAGAGTCGGAAGCCTCCACACCACCTAGCACGC
TGCCTTGCTTGTGCATCCATGCCATGACTCCAGAAAGAGTACAGCGCCTAAAAGCCTCACGTCCAGAACAAA
TTACTGTGTGCTCATCCTTTCCATTGCCGAAGTATAGAATCACTGGTGTGCAGAAGATCCAATGCTCCCAGC
CTATATTGTTCTCACCGAAAGTGCCTGCGTATATTCATCCAAGGAAGTATCTCGTGGAAACACCACCGGTAG
ACGAGACTCCGGAGCCATCGGCAGAGAACCAATCCACAGAGGGGACACCTGAACAACCACCACTTATAACCG
AGGATGAGACCAGGACTAGAACGCCTGAGCCGATCATCATCGAAGAGGAAGAAGAGGATAGCATAAGTTTGC
TGTCAGATGGCCCGACCCACCAGGTGCTGCAAGTCGAGGCAGACATTCACGGGCCGCCCTCTGTATCTAGCT
CATCCTGGTCCATTCCTCATGCATCCGACTTTGATGTGGACAGTTTATCCATACTTGACACCCTGGAGGGAG
CTAGCGTGACCAGCGGGGCAACGTCAGCCGAGACTAACTCTTACCTCGCAAAGAGTATGGAGTTTCTGGCGC
GACCGGTGCCTGCGCCTCGAACAGTATTCAGGAACCCTCCACATCCCGCTCCGCGCACAAGAACACCGTCAC
TTGCACCCAGCAGGGCCTGCTCGAGAACCAGCCTAGTTTCCACCCCGCCAGGCGTGAATAGGGTGATCACTA
GAGAGGAGCTCGAGGCGCTTACCCCGTCACGCACTCCTAGCAGGTCGGTCTCGAGAACCAGCCTGGTCTCCA
ACCCGCCAGGCGTAAATAGGGTGATTACAAGAGAGGAGTTTGAGGCGTTCGTAGCACAACAACAATGACGGT
TTGATGCGGGTGCATACATCTTTTCCTCCGACACCGGTCAAGGGCATTTACAACAAAAATCAGTAAGGCAAA
CGGTGCTATCCGAAGTGGTGTTGGAGAGGACCGAATTGGAGATTTCGTATGCCCCGCGCCTCGACCAAGAAA
AAGAAGAATTACTACGCAAGAAATTACAGTTAAATCCCACACCTGCTAACAGAAGCAGATACCAGTCCAGGA
AGGTGGAGAACATGAAAGCCATAACAGCTAGACGTATTCTGCAAGGCCTAGGGCATTATTTGAAGGCAGAAG
GAAAAGTGGAGTGCTACCGAACCCTGCATCCTGTTCCTTTGTATTCATCTAGTGTGAACCGTGCCTTTTCAA
GCCCCAAGGTCGCAGTGGAAGCCTGTAACGCCATGTTGAAAGAGAACTTTCCGACTGTGGCTTCTTACTGTA
TTATTCCAGAGTACGATGCCTATTTGGACATGGTTGACGGAGCTTCATGCTGCTTAGACACTGCCAGTTTTT
GCCCTGCAAAGCTGCGCAGCTTTCCAAAGAAACACTCCTATTTGGAACCCACAATACGATCGGCAGTGCCTT
CAGCGATCCAGAACACGCTCCAGAACGTCCTGGCAGCTGCCACAAAAAGAAATTGCAATGTCACGCAAATGA
GAGAATTGCCCGTATTGGATTCGGCGGCCTTTAATGTGGAATGCTTCAAGAAATATGCGTGTAATAATGAAT
ATTGGGAAACGTTTAAAGAAAACCCCATCAGGCTTACTGAAGAAAACGTGGTAAATTACATTACCAAATTAA
AAGGACCAAAAGCTGCTGCTCTTTTTGCGAAGACACATAATTTGAATATGTTGCAGGACATACCAATGGACA
GGTTTGTAATGGACTTAAAGAGAGACGTGAAAGTGACTCCAGGAACAAAACATACTGAAGAACGGCCCAAGG
TACAGGTGATCCAGGCTGCCGATCCGCTAGCAACAGCGTATCTGTGCGGAATCCACCGAGAGCTGGTTAGGA
GATTAAATGCGGTCCTGCTTCCGAACATTCATACACTGTTTGATATGTCGGCTGAAGACTTTGACGCTATTA
TAGCCGAGCACTTCCAGCCTGGGGATTGTGTTCTGGAAACTGACATCGCGTCGTTTGATAAAAGTGAGGACG
ACGCCATGGCTCTGACCGCGTTAATGATTCTGGAAGACTTAGGTGTGGACGCAGAGCTGTTGACGCTGATTG
AGGCGGCTTTCGGCGAAATTTCATCAATACATTTGCCCACTAAAACTAAATTTAAATTCGGAGCCATGATGA
AATCTGGAATGTTCCTCACACTGTTTGTGAACACAGTCATTAACATTGTAATCGCAAGCAGAGTGTTGAGAG
AACGGCTAACCGGATCACCATGTGCAGCATTCATTGGAGATGACAATATCGTGAAAGGAGTCAAATCGGACA
AATTAATGGCAGACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCCGTGTGGCAGACC
CCCTAAAAAGGCTGTTTAAGCTTGGCAAACCTCTGGCAGCAGACGATGAACATGATGATGACAGGAGAAGGG
CATTGCATGAAGAGTCAACACGCTGGAACCGAGTGGGTATTCTTTCAGAGCTGTGCAAGGCAGTAGAATCAA
GGTATGAAACCGTAGGAACTTCCATCATAGTTATGGCCATGACTACTCTAGCTAGCAGTGTTAAATCATTCA
GCTACCTGAGAGGGGCCCCTATAACTCTCTACGGCTAACCTGAATGGACTACGACATAGTCTAGTCCGCCAA
GTCTAGCATATGGCCACC ----------------GOI-------------------
[SEQ ID No: 458]
CGGAGACGGCGCAGAAGAAGAGGATCTGGCGAAGGCAGAGGCAGCCTGCTTACATGTGGCGACGTGGAAGAG
AACCCCGGACCTATGCACCAGAAACGGACCGCCATGTTCCAGGATCCTCAAGAGAGGCCCAGAAAGCTGCCT
CAGCTGTGTACCGAGCTGCAGACCACCATCCACGACATCATCCTGGAATGCGTGTACTGCAAGCAGCAGCTC
CTGCGGAGAGAGGTGTACGATTTCGCCTTCCGGGACCTGTGCATCGTGTACAGAGATGGCAACCCCTACGCC
GTGTGCGACAAGTGCCTGAAGTTCTACAGCAAGATCAGCGAGTACCGGCACTACTGCTACAGCCTGTACGGC
ACCACACTGGAACAGCAGTACAACAAGCCCCTGTGCGACCTGCTGATCCGGTGCATCAACTGCCAGAAACCT
CTGTGCCCCGAGGAAAAGCAGCGGCACCTGGACAAGAAGCAGCGGTTCCACAACATCAGAGGCCGGTGGACC
GGCAGATGCATGAGCTGTTGTCGGAGCAGCAGAACCAGACGGGAAACCCAGCTGTGAGCGGCCGCGAATTGG
CAAGCTGCTTACATAGAACTCGCGGCGATTGGCATGCCGCCTTAAAATTTTTATTTTATTTTTCTTTTCTTT
TCCGAATCGGATTTTGTTTTTAATATTTCAAAAAAAAAAAAAAAAAAAAAAAAAAAAAAAAAAA

Accordingly, preferably the nucleic acid sequence comprises a nucleotide sequence substantially as set out above, comprising or consisting of SEQ ID No: 93, a GOI, and SEQ ID No: 458, or a fragment or variant thereof.

In a third aspect, there is provided an expression cassette comprising a nucleic acid sequence according to the second aspect.

The nucleic acid sequences of the invention are preferably harboured in a recombinant vector, for example a recombinant vector for delivery into a host cell of interest to enable production of the RNA construct.

Accordingly, in a fourth aspect, there is provided a recombinant vector comprising the expression cassette according to the third aspect.

In one embodiment, the vector may comprise a DNA sequence which encodes or comprises an saRNA plasmid construct harbouring a GOI—furin T2A—HCV E6 (which is the first viral IIP mentioned herein, but it will be appreciated that any of the IIPs or linkers disclosed herein may be used). Therefore, the vector may comprise the nucleic acid sequence of SEQ ID No: 94, a GOI, and the nucleic acid sequence of SEQ ID No: 459, in a single vector. SEQ ID No: 94 and SEQ ID No: 459 are as follows, where “GOI” represents the position of the therapeutic biomolecule encoding sequence:

[SEQ ID No: 94]
TAATACGACTCACTATAGATGGGCGGCGCATGAGAGAAGCCCAGACCAATTACCTACCCAAAATGGAGAAAG
TTCACGTTGACATCGAGGAAGACAGCCCATTCCTCAGAGCTTTGCAGCGGAGCTTCCCGCAGTTTGAGGTAG
AAGCCAAGCAGGTCACTGATAATGACCATGCTAATGCCAGAGCGTTTTCGCATCTGGCTTCAAAACTGATCG
AAACGGAGGTGGACCCATCCGACACGATCCTTGACATTGGAAGTGCGCCCGCCCGCAGAATGTATTCTAAGC
ACAAGTATCATTGTATCTGTCCGATGAGATGTGCGGAAGATCCGGACAGATTGTATAAGTATGCAACTAAGC
TGAAGAAAAACTGTAAGGAAATAACTGATAAGGAATTGGACAAGAAAATGAAGGAGCTGGCCGCCGTCATGA
GCGACCCTGACCTGGAAACTGAGACTATGTGCCTCCACGACGACGAGTCGTGTCGCTACGAAGGGCAAGTCG
CTGTTTACCAGGATGTATACGCGGTTGACGGACCGACAAGTCTCTATCACCAAGCCAATAAGGGAGTTAGAG
TCGCCTACTGGATAGGCTTTGACACCACCCCTTTTATGTTTAAGAACTTGGCTGGAGCATATCCATCATACT
CTACCAACTGGGCCGACGAAACCGTGTTAACGGCTCGTAACATAGGCCTATGCAGCTCTGACGTTATGGAGC
GGTCACGTAGAGGGATGTCCATTCTTAGAAAGAAGTATTTGAAACCATCCAACAATGTTCTATTCTCTGTTG
GCTCGACCATCTACCACGAGAAGAGGGACTTACTGAGGAGCTGGCACCTGCCGTCTGTATTTCACTTACGTG
GCAAGCAAAATTACACATGTCGGTGTGAGACTATAGTTAGTTGCGACGGGTACGTCGTTAAAAGAATAGCTA
TCAGTCCAGGCCTGTATGGGAAGCCTTCAGGCTATGCTGCTACGATGCACCGCGAGGGATTCTTGTGCTGCA
AAGTGACAGACACATTGAACGGGGAGAGGGTCTCTTTTCCCGTGTGCACGTATGTGCCAGCTACATTGTGTG
ACCAAATGACTGGCATACTGGCAACAGATGTCAGTGCGGACGACGCGCAAAAACTGCTGGTTGGGCTCAACC
AGCGTATAGTCGTCAACGGTCGCACCCAGAGAAACACCAATACCATGAAAAATTACCTTTTGCCCGTAGTGG
CCCAGGCATTTGCTAGGTGGGCAAAGGAATATAAGGAAGATCAAGAAGATGAAAGGCCACTAGGACTACGAG
ATAGACAGTTAGTCATGGGGTGTTGTTGGGCTTTTAGAAGGCACAAGATAACATCTATTTATAAGCGCCCGG
ATACCCAAACCATCATCAAAGTGAACAGCGATTTCCACTCATTCGTGCTGCCCAGGATAGGCAGTAACACAT
TGGAGATCGGGCTGAGAACAAGAATCAGGAAAATGTTAGAGGAGCACAAGGAGCCGTCACCTCTCATTACCG
CCGAGGACGTACAAGAAGCTAAGTGCGCAGCCGATGAGGCTAAGGAGGTGCGTGAAGCCGAGGAGTTGCGCG
CAGCTCTACCACCTTTGGCAGCTGATGTTGAGGAGCCCACTCTGGAAGCCGATGTCGACTTGATGTTACAAG
AGGCTGGGGCCGGCTCAGTGGAGACACCTCGTGGCTTGATAAAGGTTACCAGCTACGATGGCGAGGACAAGA
TCGGCTCTTACGCTGTGCTTTCTCCGCAGGCTGTACTCAAGAGTGAAAAATTATCTTGCATCCACCCTCTCG
CTGAACAAGTCATAGTGATAACACACTCTGGCCGAAAAGGGCGTTATGCCGTGGAACCATACCATGGTAAAG
TAGTGGTGCCAGAGGGACATGCAATACCCGTCCAGGACTTTCAAGCTCTGAGTGAAAGTGCCACCATTGTGT
ACAACGAACGTGAGTTCGTAAACAGGTACCTGCACCATATTGCCACACATGGAGGAGCGCTGAACACTGATG
AAGAATATTACAAAACTGTCAAGCCCAGCGAGCACGACGGCGAATACCTGTACGACATCGACAGGAAACAGT
GCGTCAAGAAAGAACTAGTCACTGGGCTAGGGCTCACAGGCGAGCTGGTGGATCCTCCCTTCCATGAATTCG
CCTACGAGAGTCTGAGAACACGACCAGCCGCTCCTTACCAAGTACCAACCATAGGGGTGTATGGCGTGCCAG
GATCAGGCAAGTCTGGCATCATTAAAAGCGCAGTCACCAAAAAAGATCTAGTGGTGAGCGCCAAGAAAGAAA
ACTGTGCAGAAATTATAAGGGACGTCAAGAAAATGAAAGGGCTGGACGTCAATGCCAGAACTGTGGACTCAG
TGCTCTTGAATGGATGCAAACACCCCGTAGAGACCCTGTATATTGACGAAGCTTTTGCTTGTCATGCAGGTA
CTCTCAGAGCGCTCATAGCCATTATAAGACCTAAAAAGGCAGTGCTCTGCGGGGATCCCAAACAGTGCGGTT
TTTTTAACATGATGTGCCTGAAAGTGCATTTTAACCACGAGATTTGCACACAAGTCTTCCACAAAAGCATCT
CTCGCCGTTGCACTAAATCTGTGACTTCGGTCGTCTCAACCTTGTTTTACGACAAAAAAATGAGAACGACGA
ATCCGAAAGAGACTAAGATTGTGATTGACACTACCGGCAGTACCAAACCTAAGCAGGACGATCTCATTCTCA
CTTGTTTCAGAGGGTGGGTGAAGCAGTTGCAAATAGATTACAAAGGCAACGAAATAATGACGGCAGCTGCCT
CTCAAGGGCTGACCCGTAAAGGTGTGTATGCCGTTCGGTACAAGGTGAATGAAAATCCTCTGTACGCACCCA
CCTCAGAACATGTGAACGTCCTACTGACCCGCACGGAGGACCGCATCGTGTGGAAAACACTAGCCGGCGACC
CATGGATAAAAACACTGACTGCCAAGTACCCTGGGAATTTCACTGCCACGATAGAGGAGTGGCAAGCAGAGC
ATGATGCCATCATGAGGCACATCTTGGAGAGACCGGACCCTACCGACGTCTTCCAGAATAAGGCAAACGTGT
GTTGGGCCAAGGCTTTAGTGCCGGTGCTGAAGACCGCTGGCATAGACATGACCACTGAACAATGGAACACTG
TGGATTATTTTGAAACGGACAAAGCTCACTCAGCAGAGATAGTATTGAACCAACTATGCGTGAGGTTCTTTG
GACTCGATCTGGACTCCGGTCTATTTTCTGCACCCACTGTTCCGTTATCCATTAGGAATAATCACTGGGATA
ACTCCCCGTCGCCTAACATGTACGGGCTGAATAAAGAAGTGGTCCGTCAGCTCTCTCGCAGGTACCCACAAC
TGCCTCGGGCAGTTGCCACTGGAAGAGTCTATGACATGAACACTGGTACACTGCGCAATTATGATCCGCGCA
TAAACCTAGTACCTGTAAACAGAAGACTGCCTCATGCTTTAGTCCTCCACCATAATGAACACCCACAGAGTG
ACTTTTCTTCATTCGTCAGCAAATTGAAGGGCAGAACTGTCCTGGTGGTCGGGGAAAAGTTGTCCGTCCCAG
GCAAAATGGTTGACTGGTTGTCAGACCGGCCTGAGGCTACCTTCAGAGCTCGGCTGGATTTAGGCATCCCAG
GTGATGTGCCCAAATATGACATAATATTTGTTAATGTGAGGACCCCATATAAATACCATCACTATCAGCAGT
GTGAAGACCATGCCATTAAGCTTAGCATGTTGACCAAGAAAGCTTGTCTGCATCTGAATCCCGGCGGAACCT
GTGTCAGCATAGGTTATGGTTACGCTGACAGGGCCAGCGAAAGCATCATTGGTGCTATAGCGCGGCAGTTCA
AGTTTTCCCGGGTATGCAAACCGAAATCCTCACTTGAAGAGACGGAAGTTCTGTTTGTATTCATTGGGTACG
ATCGCAAGGCCCGTACGCACAATTCTTACAAGCTTTCATCAACCTTGACCAACATTTATACAGGTTCCAGAC
TCCACGAAGCCGGATGTGCACCCTCATATCATGTGGTGCGAGGGGATATTGCCACGGCCACCGAAGGAGTGA
TTATAAATGCTGCTAACAGCAAAGGACAACCTGGCGGAGGGGTGTGCGGAGCGCTGTATAAGAAATTCCCGG
AAAGCTTCGATTTACAGCCGATCGAAGTAGGAAAAGCGCGACTGGTCAAAGGTGCAGCTAAACATATCATTC
ATGCCGTAGGACCAAACTTCAACAAAGTTTCGGAGGTTGAAGGTGACAAACAGTTGGCAGAGGCTTATGAGT
CCATCGCTAAGATTGTCAACGATAACAATTACAAGTCAGTAGCGATTCCACTGTTGTCCACCGGCATCTTTT
CCGGGAACAAAGATCGACTAACCCAATCATTGAACCATTTGCTGACAGCTTTAGACACCACTGATGCAGATG
TAGCCATATACTGCAGGGACAAGAAATGGGAAATGACTCTCAAGGAAGCAGTGGCTAGGAGAGAAGCAGTGG
AGGAGATATGCATATCCGACGACTCTTCAGTGACAGAACCTGATGCAGAGCTGGTGAGGGTGCATCCGAAGA
GTTCTTTGGCTGGAAGGAAGGGCTACAGCACAAGCGATGGCAAAACTTTCTCATATTTGGAAGGGACCAAGT
TTCACCAGGCGGCCAAGGATATAGCAGAAATTAATGCCATGTGGCCCGTTGCAACGGAGGCCAATGAGCAGG
TATGCATGTATATCCTCGGAGAAAGCATGAGCAGTATTAGGTCGAAATGCCCCGTCGAAGAGTCGGAAGCCT
CCACACCACCTAGCACGCTGCCTTGCTTGTGCATCCATGCCATGACTCCAGAAAGAGTACAGCGCCTAAAAG
CCTCACGTCCAGAACAAATTACTGTGTGCTCATCCTTTCCATTGCCGAAGTATAGAATCACTGGTGTGCAGA
AGATCCAATGCTCCCAGCCTATATTGTTCTCACCGAAAGTGCCTGCGTATATTCATCCAAGGAAGTATCTCG
TGGAAACACCACCGGTAGACGAGACTCCGGAGCCATCGGCAGAGAACCAATCCACAGAGGGGACACCTGAAC
AACCACCACTTATAACCGAGGATGAGACCAGGACTAGAACGCCTGAGCCGATCATCATCGAAGAGGAAGAAG
AGGATAGCATAAGTTTGCTGTCAGATGGCCCGACCCACCAGGTGCTGCAAGTCGAGGCAGACATTCACGGGC
CGCCCTCTGTATCTAGCTCATCCTGGTCCATTCCTCATGCATCCGACTTTGATGTGGACAGTTTATCCATAC
TTGACACCCTGGAGGGAGCTAGCGTGACCAGCGGGGCAACGTCAGCCGAGACTAACTCTTACTTCGCAAAGA
GTATGGAGTTTCTGGCGCGACCGGTGCCTGCGCCTCGAACAGTATTCAGGAACCCTCCACATCCCGCTCCGC
GCACAAGAACACCGTCACTTGCACCCAGCAGGGCCTGCTCGAGAACCAGCCTAGTTTCCACCCCGCCAGGCG
TGAATAGGGTGATCACTAGAGAGGAGCTCGAGGCGCTTACCCCGTCACGCACTCCTAGCAGGTCGGTCTCGA
GAACCAGCCTGGTCTCCAACCCGCCAGGCGTAAATAGGGTGATTACAAGAGAGGAGTTTGAGGCGTTCGTAG
CACAACAACAATGACGGTTTGATGCGGGTGCATACATCTTTTCCTCCGACACCGGTCAAGGGCATTTACAAC
AAAAATCAGTAAGGCAAACGGTGCTATCCGAAGTGGTGTTGGAGAGGACCGAATTGGAGATTTCGTATGCCC
CGCGCCTCGACCAAGAAAAAGAAGAATTACTACGCAAGAAATTACAGTTAAATCCCACACCTGCTAACAGAA
GCAGATACCAGTCCAGGAAGGTGGAGAACATGAAAGCCATAACAGCTAGACGTATTCTGCAAGGCCTAGGGC
ATTATTTGAAGGCAGAAGGAAAAGTGGAGTGCTACCGAACCCTGCATCCTGTTCCTTTGTATTCATCTAGTG
TGAACCGTGCCTTTTCAAGCCCCAAGGTCGCAGTGGAAGCCTGTAACGCCATGTTGAAAGAGAACTTTCCGA
CTGTGGCTTCTTACTGTATTATTCCAGAGTACGATGCCTATTTGGACATGGTTGACGGAGCTTCATGCTGCT
TAGACACTGCCAGTTTTTGCCCTGCAAAGCTGCGCAGCTTTCCAAAGAAACACTCCTATTTGGAACCCACAA
TACGATCGGCAGTGCCTTCAGCGATCCAGAACACGCTCCAGAACGTCCTGGCAGCTGCCACAAAAAGAAATT
GCAATGTCACGCAAATGAGAGAATTGCCCGTATTGGATTCGGCGGCCTTTAATGTGGAATGCTTCAAGAAAT
ATGCGTGTAATAATGAATATTGGGAAACGTTTAAAGAAAACCCCATCAGGCTTACTGAAGAAAACGTGGTAA
ATTACATTACCAAATTAAAAGGACCAAAAGCTGCTGCTCTTTTTGCGAAGACACATAATTTGAATATGTTGC
AGGACATACCAATGGACAGGTTTGTAATGGACTTAAAGAGAGACGTGAAAGTGACTCCAGGAACAAAACATA
CTGAAGAACGGCCCAAGGTACAGGTGATCCAGGCTGCCGATCCGCTAGCAACAGCGTATCTGTGCGGAATCC
ACCGAGAGCTGGTTAGGAGATTAAATGCGGTCCTGCTTCCGAACATTCATACACTGTTTGATATGTCGGCTG
AAGACTTTGACGCTATTATAGCCGAGCACTTCCAGCCTGGGGATTGTGTTCTGGAAACTGACATCGCGTCGT
TTGATAAAAGTGAGGACGACGCCATGGCTCTGACCGCGTTAATGATTCTGGAAGACTTAGGTGTGGACGCAG
AGCTGTTGACGCTGATTGAGGCGGCTTTCGGCGAAATTTCATCAATACATTTGCCCACTAAAACTAAATTTA
AATTCGGAGCCATGATGAAATCTGGAATGTTCCTCACACTGTTTGTGAACACAGTCATTAACATTGTAATCG
CAAGCAGAGTGTTGAGAGAACGGCTAACCGGATCACCATGTGCAGCATTCATTGGAGATGACAATATCGTGA
AAGGAGTCAAATCGGACAAATTAATGGCAGACAGGTGCGCCACCTGGTTGAATATGGAAGTCAAGATTATAG
ATGCTGTGGTGGGCGAGAAAGCGCCTTATTTCTGTGGAGGGTTTATTTTGTGTGACTCCGTGACCGGCACAG
CGTGCCGTGTGGCAGACCCCCTAAAAAGGCTGTTTAAGCTTGGCAAACCTCTGGCAGCAGACGATGAACATG
ATGATGACAGGAGAAGGGCATTGCATGAAGAGTCAACACGCTGGAACCGAGTGGGTATTCTTTCAGAGCTGT
GCAAGGCAGTAGAATCAAGGTATGAAACCGTAGGAACTTCCATCATAGTTATGGCCATGACTACTCTAGCTA
GCAGTGTTAAATCATTCAGCTACCTGAGAGGGGCCCCTATAACTCTCTACGGCTAACCTGAATGGACTACGA
CATAGTCTAGTCCGCCAAGTCTAGCATATGGCCACC---------GOI---------
[SEQ ID No: 459]
CGGAGACGGCGCAGAAGAAGAGGATCTGGCGAAGGCAGAGGCAGCCTGCTTACATGTGGCGACGTGGAAGAG
AACCCCGGACCTATGCACCAGAAACGGACCGCCATGTTCCAGGATCCTCAAGAGAGGCCCAGAAAGCTGCCT
CAGCTGTGTACCGAGCTGCAGACCACCATCCACGACATCATCCTGGAATGCGTGTACTGCAAGCAGCAGCTC
CTGCGGAGAGAGGTGTACGATTTCGCCTTCCGGGACCTGTGCATCGTGTACAGAGATGGCAACCCCTACGCC
GTGTGCGACAAGTGCCTGAAGTTCTACAGCAAGATCAGCGAGTACCGGCACTACTGCTACAGCCTGTACGGC
ACCACACTGGAACAGCAGTACAACAAGCCCCTGTGCGACCTGCTGATCCGGTGCATCAACTGCCAGAAACCT
CTGTGCCCCGAGGAAAAGCAGCGGCACCTGGACAAGAAGCAGCGGTTCCACAACATCAGAGGCCGGTGGACC
GGCAGATGCATGAGCTGTTGTCGGAGCAGCAGAACCAGACGGGAAACCCAGCTGTGAGCGGCCGCGAATTGG
CAAGCTGCTTACATAGAACTCGCGGCGATTGGCATGCCGCCTTAAAATTTTTATTTTATTTTTCTTTTCTTT
TCCGAATCGGATTTTGTTTTTAATATTTCAAAAAAAAAAAAAAAAAAAAAAAAAAAAAAAAAAACGCGTCGA
GGGGAATTAATTCTTGAAGACGAAAGGGCCAGGTGGCACTTTTCGGGGAAATGTGCGCGGAACCCCTATTTG
TTTATTTTTCTAAATACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTTGCGGCATTTTGCCT
TCCTGTTTTTGCTCACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTGTTGACGCCGGGCAAGAGCAACTCGGTCG
CCGCATACACTATTCTCAGAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAACTCGCCTTGATCGTTG
GGAACCGGAGCTGAATGAAGCCATACCAAACGACGAGCGTGACACCACGATGCCTGTAGCAATGGCAACAAC
GTTGCGCAAACTATTAACTGGCGAACTACTTACTCTAGCTTCCCGGCAACAATTAATAGACTGGATGGAGGC
GGATAAAGTTGCAGGACCACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAGATAGGTGCCTCACTGAT
TAAGCATTGGTAACTGTCAGACCAAGTTTACTCATATATACTTTAGATTGATTTAAAACTTCATTTTTAATT
TAAAAGGATCTAGGTGAAGATCCTTTTTGATAATCTCATGACCAAAATCCCTTAACGTGAGTTTTCGTTCCA
CTGAGCGTCAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAGTGTAGCCGTAGTTAGGCCACCA
CTTCAAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAGATACCTACAGCGTGAGCA
TTGAGAAAGCGCCACGCTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAACGCCAGCAACGCGAGCTC

Accordingly, preferably the vector comprises the nucleotide sequence substantially as set out above, comprising or consisting of SEQ ID NO: 94, a GOI, and SEQ ID No: 459, or a variant or fragment thereof.

The saRNA constructs of the invention may be made using a DNA plasmid, as a template. RNA copies may then be made by in vitro transcription using a polymerase, such as T7 polymerase, and the T7 promoter may be upstream of the saRNA. Hence, the saRNA constructs of the invention may be made using the DNA plasmid having a nucleic acid sequence substantially as set out above, comprising or consisting of SEQ ID No: 94, a GOI, and SEQ ID No: 459, or a variant or fragment thereof, as the template. Of course, it will be appreciated that other RNA polymerases could be used instead of T7 polymerase, for example the SP6 or the T3 polymerase, in which case the saRNA construct may comprise the SP6 or T3 promoter instead.

The vector of the fourth aspect encoding the RNA construct of the first aspect may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the RNA sequence forming the RNA construct of the first aspect.

Recombinant vectors encoding the RNA construct of the first aspect may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell, such as a bacterial cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene(s). The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of the host cell. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells. When this is the case, regulation of expression may be less important because expression of the DNA molecule will stop when the transformed cells die or stop expressing the protein.

Alternatively, the delivery system may provide the nucleic acid molecule to the host cell without it being incorporated in a vector. For instance, the nucleic acid molecule may be incorporated within a liposome or virus particle. Alternatively a “naked” nucleic acid molecule may be inserted into a host cell by a suitable means e.g. direct endocytotic uptake.

In a fifth aspect, there is provided a pharmaceutical composition comprising the RNA construct of the first aspect, the nucleic acid sequence of the second aspect, the expression cassette of the third aspect or the vector of the fourth aspect, and a pharmaceutically acceptable vehicle.

In a sixth aspect, there is provided a process for making the pharmaceutical composition according to the fifth aspect, the method comprising contacting the RNA construct of the first aspect, the nucleic acid sequence of the second aspect, the expression cassette of the third aspect or the vector of the fourth aspect, with a pharmaceutically acceptable vehicle.

In a seventh aspect, there is provided a method of preparing the RNA construct of the first aspect, the method comprising:
a) i) introducing, into a host cell, the vector of the fourth aspect; and
   ii) culturing the host cell under conditions to result in the production of the RNA construct of the first aspect; or
b) transcribing the RNA construct from the vector according to the fourth aspect.

The host cell of step a) may be a eukaryotic or prokaryotic host cell. Preferably, the host cell is a eukaryotic host cell. More preferably, the host cell is a mammalian host cell such as Human embryonic kidney 293 cells or Chinese hamster ovary (CHO) cells. Step (b) may be performed in vitro or in vivo, preferably in vitro.

Suitable methods of in vitro transcription are well known in the art and would be known to those skilled in the art. For example, as described in Molecular Cloning, A Laboratory Manual, 2nd edition. (1989) editor C Nolan, Cold Spring Harbor Laboratory Press.

The RNA replicon of the first aspect is particularly suitable for therapy.

While the inventors envisaged that the RNA construct of the first aspect would be generated by in vitro transcription for in vivo use in therapy, those experienced in the art will recognise that the RNA construct can be generated in vivo in a subject for therapy, by in vivo delivery of the nucleic acid according to the second aspect, the expression cassette according to the third aspect, or the vector according to the fourth aspect to a subject.

Hence, according to an eighth aspect, there is provided a RNA construct according to the first aspect, the nucleic acid according to the second aspect, the expression cassette according to the third aspect, the vector according to the fourth aspect or the pharmaceutical composition according to the fifth aspect, for use as a medicament or in therapy.

In a ninth aspect of the invention, there is provided a RNA construct according to the first aspect, the nucleic acid according to the second aspect, the expression cassette according to the third aspect, the vector according to the fourth aspect or the pharmaceutical composition according to the fifth aspect, for use in the prevention, amelioration or treatment of a protozoan, fungal, bacterial or viral infection.

The protozoan, fungal, bacterial or viral infection may be an infection of a protozoa, fungus, bacterium or virus as defined in the first aspect.

In a tenth aspect of the invention, there is provided an RNA construct according to the first aspect, the nucleic acid according to the second aspect, the expression cassette according to the third aspect, the vector according to the fourth aspect or the pharmaceutical composition according to the fifth aspect, for use in the prevention, amelioration or treatment of cancer.

The cancer may be as defined in the first aspect.

In an eleventh aspect of the invention, there is provided a method for treating a protozoan, fungal, bacterial or viral infection, the method comprising administering, to a subject in need thereof, a therapeutically effective amount of the RNA construct according to the first aspect, the nucleic acid according to the second aspect, the expression cassette according to the third aspect, the vector according to the fourth aspect or the pharmaceutical composition according to the fifth aspect.

The protozoan, fungal, bacterial or viral infection to be treated may be an infection of a protozoa, fungus, bacterium or virus as defined in the first aspect.

In a twelfth aspect of the invention, there is provided a method for treating cancer, the method comprising administering, to a subject in need thereof, a therapeutically effective amount of the RNA construct according to the first aspect, the nucleic acid according to the second aspect, the expression cassette according to the third aspect, the vector according to the fourth aspect or the pharmaceutical composition according to the fifth aspect.

The cancer to be treated may be as defined in the first aspect.

The RNA construct described herein provides an effective means of vaccinating a subject (e.g. against a viral, bacterial or fungal infection) and cancer.

Accordingly, in a thirteenth aspect of the invention, there is provided a vaccine comprising the RNA construct according to the first aspect, the nucleic acid according to the second aspect, the expression cassette according to the third aspect, the vector according to the fourth aspect or the pharmaceutical composition according to the fifth aspect.

The adjuvant incorporated into a delivery formulation may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Tomatine, ISCOM, ISCOMATRIX™, squalene based emulsions, polymers such as PEI, Carbopol, lipid nanoparticles and bacterial toxins (CT, LT). Other examples of adjuvants incorporated into the delivery formulation may include an aluminium salt, a synthetic form of DNA, a carbohydrate, a tablet binder, an ion exchange resin, a preservative, a polymer, an emulsion and/or a lipid. Examples of adjuvants may include monosodium glutamate, sucrose, dextrose, aluminum bovine, human serum albumin, cytosine phosphoguanine, potassium phosphate, plasdone C, anhydrous lactose, cellulose, polacrilin potassium, glycerine, asparagine, citric acid, potassium phosphate magnesium sulfate, iron ammonium citrate, 2-phenoxyethanol, aluminium, beta-propiolactone, bovine extract, DOPC, EDTA, formaldehyde, thimerosal, phenol, potassium aluminum sulfate, potassium glutamate, sodium borate, sodium metabisulphite, urea, PLGA, PVA, PLA, PVP, cyclodextrin-based stabilisers, oil in water emulsion adjuvants and/or lipid-based adjuvants.

In a fourteenth aspect of the invention, there is provided an RNA construct according to the first aspect, the nucleic acid according to the second aspect, the expression cassette according to the third aspect, the vector according to the fourth aspect or the pharmaceutical composition according to the fifth aspect, for use in stimulating an immune response in a subject.

The immune response may be stimulated against a protozoa, bacterium, virus, fungus or cancer as per the antigens defined in the first aspect.

In another aspect, there is provided a method of vaccinating a subject, the method comprising administering, or having administered, to a subject in need thereof, a therapeutically effective amount of the first aspect, the nucleic acid according to the second aspect, the expression cassette according to the third aspect, the vector according to the fourth aspect or the pharmaceutical composition according to the fifth aspect.

According to a fifteenth aspect, there is provided an RNA construct according to the first aspect, the nucleic acid according to the second aspect, the expression cassette according to the third aspect, the vector according to the fourth aspect or the pharmaceutical composition according to the fifth aspect, for use in stem cell therapy.

Stem cell therapy may relate to the reprogramming somatic cells to cells having stem cell characteristics.

Somatic cells may be reprogrammed by delivering one or more proteins that are capable of enhancing reprogramming of somatic cells to cells having stem cell characteristics as defined in the first aspect.

According to a sixteenth aspect, there is provided a method of modifying a cell ex vivo or in vitro, comprising delivering, to the cell, the RNA construct according to the first aspect, the nucleic acid according to the second aspect, the expression cassette according to the third aspect, the vector according to the fourth aspect or the pharmaceutical composition according to the fifth aspect.

Preferably, the method is performed ex vivo.

The cell may be a eukaryotic or prokaryotic cell. Preferably, the cell is a eukaryotic cell. More preferably, the cell is a mammalian host cell. Most preferably, the cell is a human cell.

Preferably, the modified cell is suitable for cell-therapy indications.

In a seventeenth aspect, there is provided a modified cell obtained from, or obtainable by, the method of the sixteenth aspect.

In an eighteenth aspect, there is provided the modified cell of the seventeenth aspect, for use in therapy, optionally cell therapy.

It will be appreciated that the RNA construct according to the first aspect, the nucleic acid according to the second aspect, the expression cassette according to the third aspect, the vector according to the fourth aspect or the pharmaceutical composition according to the fifth aspect (herein known as the active agents) may be used in a medicament, which may be used as a monotherapy (i.e. use of the active agent), for treating, ameliorating, or preventing disease or vaccination. Alternatively, the active agents according to the invention may be used as an adjunct to, or in combination with, known therapies for treating, ameliorating, or preventing disease.

The RNA construct, nucleic acid sequence, expression cassette, vector or pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles (with RNA on the surface or encapsulated) or any other suitable form that may be administered to a person or animal in need of treatment or vaccination. It will be appreciated that the vehicle of medicaments according to the invention should be one which is well-tolerated by the subject to whom it is given.

The RNA construct, nucleic acid sequence, expression cassette, vector or pharmaceutical composition of the invention may also be incorporated within a slow- or delayed-release device. Such devices may, for example, be inserted on or under the skin, and the medicament may be released over weeks or even months. The device may be located at least adjacent to the treatment site. Such devices may be particularly advantageous when long-term treatment with the RNA construct or the recombinant vector is required and which would normally require frequent administration (e.g. at least daily injection).

In a preferred embodiment, however, medicaments according to the invention may be administered to a subject by injection into the blood stream, muscle, skin or directly into a site requiring treatment. Most preferably, the medicaments, including the RNA construct, are injected into muscle. Injections may be intravenous (bolus or infusion) or subcutaneous (bolus or infusion), or intradermal (bolus or infusion), or intramuscular (bolus or infusion).

It will be appreciated that the amount of RNA construct, nucleic acid sequence, expression cassette, vector or pharmaceutical composition that is required is determined by its biological activity and bioavailability, which in turn depends on the mode of administration, the physiochemical properties of the RNA construct, nucleic acid sequence, expression cassette, vector or pharmaceutical composition and whether it is being used as a monotherapy or in a combined therapy. The frequency of administration will also be influenced by the half-life of the active agent within the subject being treated. Optimal dosages to be administered may be determined by those skilled in the art, and will vary with the particular the RNA construct, nucleic acid sequence, expression cassette, vector or pharmaceutical composition in use, the strength of the pharmaceutical composition, the mode of administration, and the type and advancement of the viral infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.1 μg/kg of body weight and 1 mg/kg of body weight, of the RNA construct, nucleic acid sequence, expression cassette, vector or pharmaceutical composition of the invention may be used for treating, ameliorating, or preventing a disease, depending upon the active agent used.

Daily doses may be given as a single administration (e.g. a single daily injection or inhalation of a nasal spray). Alternatively, the RNA construct, nucleic acid sequence, expression cassette, vector or pharmaceutical composition may require administration twice or more times during a day. As an example, the RNA construct, nucleic acid sequence, expression cassette, vector or pharmaceutical composition may be administered as two (or more depending upon the severity of the disease being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the RNA construct, nucleic acid sequence, expression cassette, vector or pharmaceutical composition according to the invention to a patient without the need to administer repeated doses.

Preferably, however, the RNA construct, nucleic acid sequence, expression cassette, vector or pharmaceutical composition according to the invention may be given as a weekly dose, and more preferably a fortnightly dose.

Known procedures, such as those conventionally employed by the pharmaceutical industry (e.g. in vivo experimentation, clinical trials, etc.), may be used to form specific formulations of the RNA construct, nucleic acid sequence, expression cassette or vector according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the RNA construct, nucleic acid sequence, expression cassette, vector or pharmaceutical composition is any amount which, when administered to a subject, is the amount of the aforementioned that is needed to ameliorate, prevent or treat any given disease.

For example, the RNA construct, nucleic acid sequence, expression cassette, vector or pharmaceutical composition of the invention may be used from about 0.0001 mg to about 800 mg, and preferably from about 0.001 mg to about 500 mg. It is preferred that the amount of the replicon, nucleic acid sequence, expression cassette, vector or pharmaceutical composition is an amount from about 0.001 mg to about 250 mg, and most preferably from about 0.01 mg to about 1 mg. Preferably, the RNA construct, nucleic acid sequence, expression cassette, vector or pharmaceutical composition according to the invention is administered at a dose of 1-200 g.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. RNA construct, nucleic acid sequence, expression cassette, vector or pharmaceutical composition according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, tale, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The RNA construct, nucleic acid sequence, expression cassette, vector or pharmaceutical composition according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The nucleic acid sequence, or expression cassette of the invention may be prepared as a sterile solid composition that may be dissolved or suspended at the time of administration using sterile water, saline, or other appropriate sterile injectable medium.

The RNA construct, nucleic acid sequence, expression cassette, vector or pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The RNA construct, nucleic acid sequence, expression cassette, vector or pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequences identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from any of the sequences described herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows a schematic of various embodiments (denoted 1-7) of the RNA construct of the invention (e.g. a saRNA replicon on the left, or a mRNA construct). The saRNA replicon (1-4) is based on an alpha virus backbone. This so-called ‘Stealthicon’ vector includes a 5′ UTR followed by nucleic acid encoding Non-structural Proteins (NSP1-4) from an alphavirus, such as VEEV, a sub-genomic promoter (SGP), a GOI (Gene of Interest), such as a viral, bacterial, fungal or mammalian protein or antigen, a viral innate inhibitor protein (IIP), a 3′ UTR and a 3′ poly A tail. The mRNA construct (5-7) includes a 5′ UTR, a GOI (Gene of Interest), such as a viral, bacterial, fungal or mammalian protein or antigen, a viral innate inhibitor protein (IIP), a 3′ UTR and a 3′ poly A tail. The order of the IIP and GOI can be varied for both saRNA and mRNA as shown in the different illustrated embodiments;

FIG. 2 illustrates the immune response in a subject vaccinated (an initial primer jab followed by a subsequent boost jab) with a messenger RNA (mRNA) vaccine;

FIG. 3 illustrates the immune response in a subject vaccinated (an initial primer jab followed by a boost jab) with a standard self-amplifying (saRNA) vaccine;

FIG. 4 illustrates the immune response in a subject vaccinated (an initial primer jab followed by a boost jab) with one embodiment of the RNA construct of the invention, for example the Stealthicon vector shown in FIG. 1;

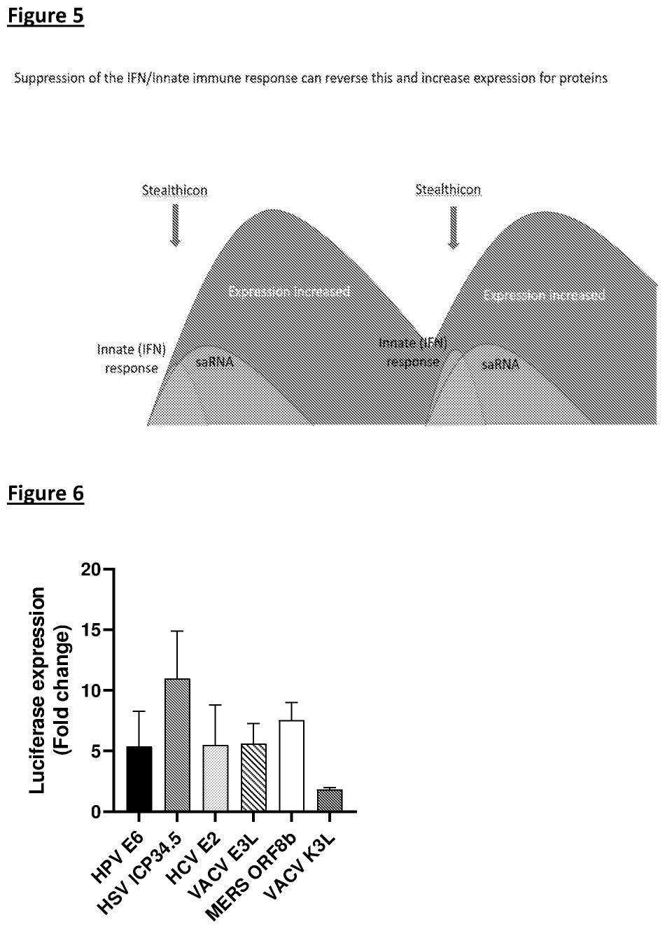

FIG. 5 illustrates the antigen expression level in a subject vaccinated (an initial primer jab followed by a boost jab) with one embodiment of the RNA construct of the invention, i.e. the Stealthicon vector shown in FIG. 1;

FIG. 6 shows f-Luc expression in HeLa cells following transfection with VEEV replicons containing selected IIP in F-T2A configuration relative to expression in HEK293T/17 cells. HEK293T/17 and HeLa cells were transfected with saRNA (100 ng) containing luciferase as a reporter protein and assessed for protein expression after 24 hr;

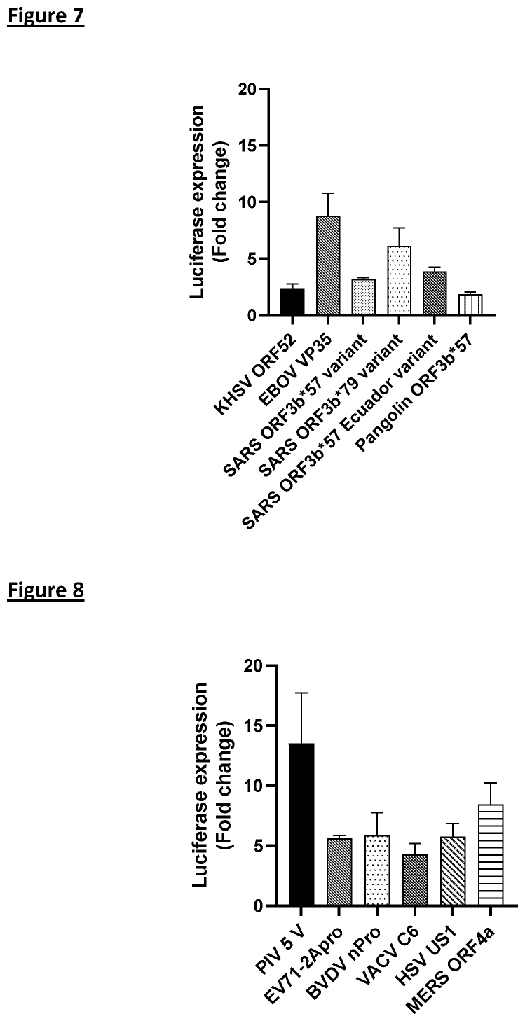

FIG. 7 shows f-Luc expression in HeLa cells following transfection with VEEV replicons containing selected IIP in F-T2A configuration relative to expression in HEK293T/17 cells. HEK293T/17 and HeLa cells were transfected with saRNA (100 ng) containing luciferase as a reporter protein and assessed for protein expression after 24 hr;

FIG. 8 shows f-Luc expression in HeLa cells following transfection with selected VEEV replicons containing IP in F-T2A configuration relative to expression in HEK293T/17 cells. HEK293T/17 and HeLa cells were transfected with saRNA (100 ng) containing luciferase as a reporter protein and assessed for protein expression after 24 hr;

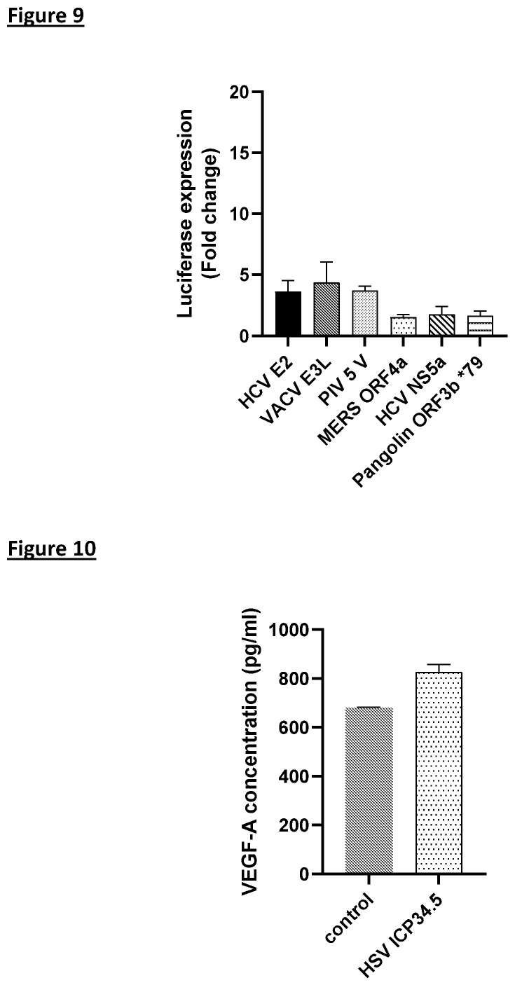

FIG. 9 shows f-Luc expression in HeLa cells following transfection with VEEV replicons containing IIP in a double sub-genomic promoter (DSGP) configuration relative to expression in HEK293T/17 cells. HEK293T/17 and HeLa cells were transfected with saRNA (100 ng) containing luciferase as a reporter protein and assessed for protein expression after 24 hr;

FIG. 10 shows the increase in VEGF-A expression in HeLa cells following transfection with saRNA containing the IIP HSV ICP34.5 in a F-T2A configuration compared to saRNA without IIP and relative to expression in HEK293T/17 cells. HEK293T/17 and HeLa cells were transfected with saRNA (100 ng) containing VEGF-A as a secreted reporter protein and assessed for protein expression in the culture media after 48 hr by ELISA;

FIG. 11 compares f-Luc expression in HeLa cells following transfection with saRNA containing f-Luc as the GOI (construct 1) and with the IIP MERS ORF4a in F-T2A configuration (constructs 2a and 2b), IRES configuration (construct 3b) and DSGP configurations (constructs 4a and 4b) shown in FIG. 1. HeLa cells were transfected with RNA (100 ng) containing luciferase as a reporter protein and assessed for protein expression after 24 hr, and

FIG. 12 shows n-Luc expression f-Luc expression in HeLa cells following transfection with mRNA containing IIP in F-T2A configuration relative to expression in HEK293T/17 cells. HEK293T/17 and HeLa cells were transfected with RNA (100 ng) containing luciferase as a reporter protein and assessed for protein expression after 24 hr.

### EXAMPLES

The inventors hypothesized that cis encoding proteins from non-viral sources, such as humans and other mammals, that are known to inhibit the innate recognition of saRNA or mRNA, would dampen the innate sensing in the host cell, and enhance both the protein expression and immunogenicity of RNA vaccines. Thus, the inventors designed and tested a range of RNA constructs (saRNA and mRNA) containing viral innate immune inhibitor proteins (IIPs) and a gene of interest (GOI), and then characterized whether these constructs enhance both intracellular and secreted protein expression (encoded by the gene of interest).

Materials and Methods

Cloning of saRNA Replicon Plasmids Containing IIPs

SaRNA encoding firefly luciferase (fLuc) and replicase derived from the Venezuelan equine encephalitis virus (VEEV) were cloned into a plasmid vector, as previously described (1). Replicon plasmids containing reporter gene followed by IIP (firefly luciferase f-Luc; Uniprot: Q27758) were generated with Furin-T2A or double sub-genomic promoters. Double sub-genomic (DSG) constructs are designed to initiate transcription of separate RNA molecules encoding the fLuc and IIP and were produced by cloning into a base double sub-genomic vector using Gibson assembly and a nucleotide base overlap. Briefly, plasmid DNA was restriction digested for 2 h at 37° C. and used in a NEB Builder HiFi DNA assembly reaction with gene fragment strings synthesised by GeneArt (Regensburg, Germany) or Integrated DNA Technologies (IDT) (Iowa, USA) according to manufacturer's protocol (New England BioLabs, UK). Furin-T2A (F-T2A) constructs designed to generate a single RNA transcript from the VEEV primary sub-genomic promoter with no stop codon for fLuc translation were produced by cloning IIP with F-T2A sequence into restriction enzyme sites of the corresponding DSG plasmid vector. After incubation at 50° C. for 30 min, 2 uL of the NEB Builder HiFi assembly reaction was used to transform NEB 10-alpha bacteria and the transformants plated onto LB agar plates and incubated overnight. Colonies were selected, expanded overnight and recombinant plasmid purified using Qiagen plasmid miniprep kits (Qiagen, UK). Purified clonal plasmids were analysed using a diagnostic restriction enzyme digest and those which exhibited the correct digestion pattern were fully sequenced to confirm nucleotide identity (Eurofins, Germany).

Plasmids that had IIP followed by the reporter gene in the F-T2A or DSG form as well as constructs that utilized the ECMV IRES internal ribosomal entry sequence (which initiates protein translation from the IRES element at a site internal to a messenger RNA transcript; Bochkov and Palmenburg, Biotechniques 41(3):283-4, 2006) were generated by VectorBuilder (VectorBuilder, Germany) using standard molecular techniques.

The incorporated interferon inhibiting proteins (IIP) can be found with the following database identifiers/accession numbers: EBOV VP35 (Ebola virus VP35; NP_066244.1; Accession Number—NCBI Reference Sequence: NC_002549.1; UniProtKB—Q05127 (VP35_EBOZM); EV71-2Apro (Enterovirus 71 2A pro; Accession Numbers—GenBank KC875402.1 and AG028195.1; UniProtKB—Q66478 (POLG_HE71B); HCV E2 (hepatitis C virus E2; NS1 Protein from polyprotein AAA45534.1; Accession Number—Genomic RNA Translation AAA45534.1; UniProtKB—P27958 (384-746) (POLG_HCV77)); HCV NS5a (hepatitis C virus NS5a; isolate H—Genomic RNA translation: AAA45534.1; UniProtKB—P27958 (POLG_HCV77)); HPV E6 (Human papillomavirus E6; NP_041325.1; Accession Number—NCBI Reference Sequence: NC_001526.4; UniProtKB—P03126 (VE6_HPV16)); HSV ICP34.5 (Herpes simplex virus ICP34.5; YP_009137073.1; Accession Number—NCBI Reference Sequence: NC_001806.2; UniProtKB—P36313 (ICP34_HHV11)); KSHV ORF52 (Kaposi's sarcoma-associated herpesvirus ORF52; Accession Number—Genomic DNA Translation: ACY00451.1; UniProtKB—F5HBL8 (F5HBL8_HHV8)); MERS ORF8b (Middle East Respiratory Syndrome virus ORF8b; Accession Number—Genomic RNA Translation ANF29170.1; UniProtKB—A0A1W5LGP6 (A0A1W5LGP6_MERS)); VACV C6 Vaccinia C6 (vaccinia virus C6; Accession Number—Genomic DNA Translation: AAA69602.1; UniProtKB—P17362 (C6_VACCW)); VACV K3L (vaccinia virus K3L; Accession Number—Genomic DNA Translation: AAA48009.1; UniProtKB—P20639 (K3 VACCC)); PIV 5 V (Parainfluenza virus 5 V; ENA protein ID: AAA47882.1; GenBank Accession Number J03142.1; UniProtKB—P11207; V_PIV5)); SARS ORF3b*57 variant (Severe acute respiratory syndrome coronavirus 2 (2019-nCoV) (SARS-CoV-2) ORF3b protein-mutated stop codon at AA 23; Genomic RNA Translation QTT40181.1; UniProtKB—PoDTF1 (ORF3B_SARS2)); SARS ORF3b*79 variant (Severe acute respiratory syndrome coronavirus 2 (2019-nCoV) (SARS-CoV-2) ORF3b protein-mutated stop codons at AA 23 and AA 57; Genomic RNA Translation QTT40181.1; UniProtKB—PoDTF1 (ORF3B_SARS2)); SARS ORF3b*57 Ecuador variant (Severe acute respiratory syndrome coronavirus 2 (2019-nCoV) (SARS-CoV-2) ORF3b protein-mutated stop codon at AA 23; Ecuador mutation at AA 24 (L24M); Genomic RNA Translation QTT40181.1; UniProtKB—PoDTF1 (ORF3B_SARS2)); Pangolin ORF3b *57 (Pangolin Coronavirus—Genomic RNA Translation: QIG55946.1; ORF3b protein-mutated stop codon at AA 23; UniProtKB—A0A6M3G7Q4 (A0A6M3G7Q4_9BETC)); Pangolin ORF3b *79 (Pangolin Coronavirus—Genomic RNA Translation: QIG55946.1; ORF3b protein-mutated stop codons at AA 23 and AA 57; UniProtKB—A0A6M3G7Q4 (A0A6M3G7Q4_9BETC)); MERS ORF4a (Middle East respiratory syndrome-related coronavirus (MERS-CoV) NS4A protein—Genomic RNA Translation: AGV08457.1; UniProtKB: T2BBG6 (T2BBG6_MERS)); BVDV nPro (Bovine viral diarrhea virus (BVDV) (Mucosal disease virus) N-terminal protease (aa 1-168)—Genomic RNA Translation: AAA42854.1; UniProtKB: P19711 (POLG_BVDVN)); HSV US1 (Human herpesvirus 2 (strain HG52) (HHV-2) (Human herpes simplex virus 2) E3 ubiquitin ligase ICP22 US1—Genomic DNA Translation: CAB06708.1; UniProtKB: P89474 (ICP22_HHV2H)); MERS CoV M (Middle East respiratory syndrome-related coronavirus (MERS-CoV) Membrane protein (M)-Genomic RNA Translation: AGV08396.1; UniProtKB: T2BB40 (T2BB40_MERS)).
(1) A. K. Blakney, P. F. McKay, R. J. Shattock, Structural Components for Amplification of Positive and Negative Strand VEEV Splitzicons. Frontiers in Molecular Biosciences 5, 71 (2018).

Cloning of Plasmids Containing IIPs for RNA Transcription

IIP were inserted into a base plasmid using restriction digestion followed by Gibson assembly with a nucleotide base overlap region and included a F-T2A sequence to allow for a single transcript expression of the n-Luc followed by an IIP. The base plasmid consisted of an mRNA encoding a luminous shrimp nanoluciferase (n-Luc) expression cassette with a T7 promoter, an alpha-globin 5′ UTR and a beta-globin 3′ UTR. Briefly, the n-Luc plasmid construct was linearized with restriction enzymes for 2 h at 37° C. and then used in a NEB Builder HiFi DNA assembly reaction essentially as described in the NEB Builder HiFi assembly protocol (New England BioLabs, UK). After incubation at 50° C. for 30 min, 2 uL of the assembly reaction was used to transform NEB 10-alpha bacteria as per protocol and the transformants plated onto LB agar plates and incubated overnight for colony growth. Colonies were selected and expanded overnight, the recombinant plasmid purified from the bacteria using Qiagen plasmid miniprep kit (Qiagen, UK) and purified clonal plasmids were analysed initially using a diagnostic restriction enzyme digest and those which exhibited the correct digestion pattern were fully sequenced to confirm nucleotide identity (Eurofins, Germany).

Plasmids that had IIP followed by the n-Luc in the F-T2A or DSG form as well as constructs that utilized the ECMV IRES internal ribosomal entry sequence (which initiates protein translation from the IRES element at a site internal to a messenger RNA transcript; Bochkov and Palmenburg, Biotechniques 41(3):283-4, 2006) for both the saRNA replicons and the plasmids used for mRNA transcription were generated by VectorBuilder (VectorBuilder, Germany) using standard molecular techniques.

In Vitro Transcription of saRNA

Plasmid DNA (pDNA) was transformed into Escherichia coli (E. coli) (New England BioLabs, UK) and cultured in 100 mL of Luria Broth (LB) with 100 μg/mL of carbenicillin (Sigma Aldrich, UK). pDNA was isolated using a Plasmid Plus MaxiPrep kit (QIAGEN, UK) and the final concentration measured on a NanoDrop One (ThermoFisher, UK). saRNA was transcribed from the pDNA template using CleanCap Reagent AG (Tebu-bio, France) to produce an RNA transcript with a naturally occurring Cap 1 structure. Briefly, the pDNA template was linearized for 3 h at 37° C., then 1 μg of the linearized pDNA template used in the standard CleanCap Transcription protocol (Tebu-bio, France) according to the manufacturer's protocol. Transcripts were purified by LiCl precipitation at −20° C. for at least 30 min, centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK) and stored at −80° C. until further use.

In Vitro Transcription of RNA

pDNA was transformed into E. coli (New England BioLabs, UK), cultured in 100 mL of Luria Broth (LB) with 100 μg/mL of carbenicillin (Sigma Aldrich, UK). Plasmid was purified using a Plasmid Plus MaxiPrep kit (QIAGEN, UK) and the concentration and purity measured on a NanoDrop One (ThermoFisher, UK). RNA was transcribed from the plasmid DNA template using the MEGAscript™ T7 Transcription protocol (ThermoFisher, UK) followed by a ScriptCap™ m7G Capping System post translation (Cambio, UK). Briefly, pDNA was linearized for 3 h at 37° C., and 1 μg of the linearized pDNA template used in the standard reaction protocol. After the MEGAscript™ T7 Transcription the transcripts were purified by LiCl precipitation at −20° C. for at least 30 min, then centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK). The transcripts were then post-transcriptionally capped using the ScriptCap™ m7G Capping System standard protocol and finally LiCl precipitated as described above. Purified and Cap 1 capped RNA was then resuspended in UltraPure H₂O (Ambion, UK) and stored at −80° C. until further use.

Measurement of IIP Activity

In order to establish the ability of saRNA containing viral IIP to increase saRNA f-luc expression relative to saRNA without IIP; the ability of mRNA containing IIP to increase mRNA n-luc expression relative to mRNA without IIP and the ability of mRNA containing IIP to increase f-luc expression from saRNA without IIP, constructs were tested in interferon competent HeLa cells and expression compared to that obtained in HEK293T/17 cells which do not have a functional antiviral signalling pathway. Both cell lines were cultured in high glucose Dulbecco's Modified Eagle's Medium (cDMEM) (Sigma-Aldrich, Merck, UK) containing 10% (v/v) fetal bovine serum (FBS), 5 mg/mL L-glutamine (Gibco, ThermoFisher, UK) and 5 mg/mL penicillin/streptomycin (Sigma-Aldrich, Merck, UK).

Assessment of IIP on saRNA Firefly Luciferase (f-Luc) Expression

HEK293T/17 cells were plated at a density of 25000 cells per well and HeLa cells at a density of 10000 cells per well into flat clear bottom 96-well plates (Corning Costar) and incubated for 24 hr. 10 uL of OptiMEM (ThermoFisher, UK) containing 0.15 μL lipofectamine MessengerMAX (ThermoFisher, UK) and 100 ng of saRNA IIP constructs or saRNA control (no IIP) was added to triplicate wells and after a further 24 hr, plates were centrifuged at 630 g for 5 min at room temperature, 50 μL of medium removed from each well and 50 μL of ONE-Glo™ Ex Reagent D-luciferin reagent (Promega, UK) added and mixed by pipetting. The total volume from each well was then transferred to a flat bottom opaque white 96-well plate (Corning Costar) and fluorescence measured on a FLUOstar OMEGA plate reader within 10 min (BMG LABTECH, UK). Background fluorescence from control wells containing no saRNA was subtracted from the signal for each well containing saRNA. Then the signal obtained for saRNA containing IIP in HeLa cells was expressed as a fold change from signal obtained with control saRNA and to that obtained in HEK293T/17 cells.

Assessment of IIP on saRNA VEGF-A Expression

HEK293T/17 or Hela cells were transfected with 100 ng saRNA containing the VEGF-A gene using the same methods as described for testing of constructs expressing f-Luc. After 48 hr the VEGF-A in the cell culture media was measured using a human VEGF-A ELISA kit (Invitrogen, UK). Briefly, assay plate wells were washed twice with 400 uL wash buffer before addition of test samples or VEGF-A standard (15.6 μg/ml to 1000 μg/ml). Plates were then incubated at room temperature for 2 hr in a microplate shaker (300 rpm; Jencons Scientific Ltd, UK) before washing six times with 400 uL wash buffer 100 uL of Biotin-conjugate detection antibody (1:100 dilution) was added to each well and plates incubated in a microplate shaker (1 hr RT, 300 rpm). After six washes with 400 uL of wash buffer, the streptavidin-HRP (1:100 dilution) second layer conjugate (100 uL) was added and after a further 1 hr incubation and six further washes, 100 uL of TMB substrate was added to each well. After incubation in the dark for 30 min at RT in the dark, 100 uL of the Stop solution was added and the absorbance of each well read at 450 nm in a VersaMax microplate spectrophotometer (Molecular Devices, UK). VEGF-A levels in the samples were determined by interpolation to the standard curve.

Assessment of IIP on RNA Nano-Luciferase (n-Luc) Expression

HEK293T/17 cells were plated at a density of 25000 cells per well and HeLa cells at a density of 10000 cells per well into flat clear bottom 96-well plates (Corning Costar) and incubated for 24 hr. 10 uL of OptiMEM (ThermoFisher, UK) containing 0.15 μL lipofectamine MessengerMAX (ThermoFisher, UK) and 100 ng of saRNA IIP constructs or saRNA control (no IIP) was added to triplicate wells and after a further 24 hr, plates were centrifuged at 630 g for 5 min at room temperature, 50 μL of medium removed from each well and 50 μL of NanoDLR™ Stop & Glo® Reagent (Promega, UK) added and mixed by pipetting. The total volume from each well was then transferred to a flat bottom opaque white 96-well plate (Corning Costar) and fluorescence measured on a FLUOstar® OMEGA plate reader within 10 min (BMG LABTECH, UK). Background fluorescence from control wells containing no RNA was subtracted from the signal for each well containing RNA. Then the signal obtained for RNA containing IIP in HeLa cells was expressed as a fold change from signal obtained with control RNA and to that obtained in HEK293T/17 cells.

### Example 1—Structural Design of Viral Innate Inhibitor Protein (IIP) Constructs

Viral innate inhibitor proteins (IIPs) can be incorporated into an RNA construct of the invention, which can be a self-amplifying RNA (saRNA) or messenger RNA (mRNA), in order to reduce or ablate the innate recognition and response that may modify or reduce protein expression and translation, i.e. the protein encoded by a Gene of Interest (GOI), which can be any therapeutic biomolecule.

Various embodiments of design configurations for the RNA construct of the invention are shown in FIG. 1. SaRNA expression constructs are based on an alphavirus backbone where the non-structural proteins are maintained, but the gene of interest (GOI) is inserted downstream of a subgenomic promotor (SGP) replacing the structural genes of the virus (see Embodiment “1” in FIG. 1). The GOI can be any protein at all, and may include viral, bacterial, fungal or mammalian protein, i.e. a biotherapeutic protein. However, the inventors envisage that the RNA construct of the invention will demonstrate significant utility in the vaccine space, and so the GOI would encode a vaccine antigen, such as a viral, bacterial or fungal protein, such as a coat protein.

saRNA Constructs (Left Hand of FIG. 1)

Any IIP can be encoded within the saRNA using the following design approaches:
Embodiment “2a” in FIG. 1 shows a saRNA construct encoding a fusion protein including a peptide cleavage motif (e.g. furin-T2a), such that the protein encoded by the GOI (e.g. the antigen of interest) and the IIP are cleaved into separate proteins on translation in the host cell;
In Embodiment “2b” in FIG. 1, the order of the GOI and IIP have been reversed, such that the IIP is 5′ of the GOI, again with a peptide cleavage motif between the IIP and the GOI so that two separate proteins are produced in the host cell following translation of the saRNA construct;
In Embodiment “3a”, the IIP has been inserted downstream of the GOI stop codon. The subgenomic promoter drives translation of the GOI, and expression/translation of the IIP is driven by the inclusion of an internal ribosomal entry site (IRES);
In Embodiment “3b”, the order of the GOI and IIP has been reversed such that translation of the IIP is promoted by the subgenomic promotor and of the GOI by the IRES;
In Embodiment “4a”, the IIP has been inserted downstream of the GOI stop codon. Translation of the GOI is promoted by the first subgenomic promoter and translation of the IIP is driven by the inclusion of a second subgenomic promotor;
In Embodiment “4b”, the position of the IIP and GOI have been swapped around, i.e. with the IIP placed before the GOI.

mRNA Constructs (Right Hand of FIG. 1)

Referring to FIG. 1, any IIP can also be encoded within mRNA (see Embodiment “5”) using the following design approaches:
In embodiment “6a”, the mRNA construct encodes a fusion protein including a peptide cleavage motif (e.g. F-T2a) such that the GOI and IIP are cleaved into separate proteins on translation;
In Embodiment “6b”, the order of the GOI and IIP have been reversed such that the IIP is 5′ of the GOI;
In Embodiment “7a”, the IIP has been inserted downstream of the GOI stop codon where translation is driven by the inclusion of an internal ribosomal entry site (IRES);
In Embodiment “7b”, the order of the GOI and IIP has been reversed such that translation is promoted by the subgenomic promotor and the GOI by the IRES.

The inventors have tested a large number of viral IIPs in the various embodiments of RNA constructs illustrated in FIG. 1, and believe that they each have potential to modify expression and response to saRNA and RNA.

### Example 2—Construction and Testing of saRNA Constructs Comprising a Viral Innate

Inhibitor Protein (IIP) The inventors designed, constructed and then tested a series of diverse viral IIPs in different replicon configurations on expression of the reporter gene, f-Luc or VEGF-A, and the results of the expression studies are shown in FIGS. 6-11.

Referring to FIG. 6, there is shown the fold increase in f-Luc expression in HeLa cells following transfection with VEEV replicons containing HPV E6, HSV ICP34.5, HCV E2, VACV E3L, MERS ORF8b or VACV K3L in an F-T2A configuration. HEK293T/17 and HeLa cells were transfected with saRNA (100 ng) containing luciferase as a reporter protein and assessed for protein expression after 24 hr. HeLa cells are known to have more intact IFN expression pathways compared to HEK293T/17 and therefore increased expression (fold increase) relative to a control (saRNA containing luciferase as reporter protein and no IIP) indicates that the IIP is increasing saRNA expression. Of these IIP, HSV ICP34.5 produced the greatest increase in f-Luc expression. Data shown are constructs providing a greater than −2-fold increase in luciferase expression in HeLa cells relative to expression in HEK293T/17 cells and are mean f SEM of data obtained in 3 independent experiments using 3 separate batches of saRNA.

Referring to FIG. 7, there is shown f-Luc expression in HeLa cells following transfection with VEEV replicons containing KHSV ORF52, EBOV VP35, SARS ORF3b* 57 variant, SARS ORF3b*79 variant, SARS ORF3b*57 Equador variant or Pangolin ORF3b* 57 in an F-T2A configuration relative to expression in HEK293T/17 cells. Details of experimental methods are provided in FIG. 6. Of these EBOV VP35 and SARS ORF3b*79 variant produced the greatest increase in f-Luc expression. Data shown are constructs providing a greater than 2-fold increase in luciferase expression in HeLa cells relative to expression in HEK293T/17 cells and are mean f SEM of data obtained in 3 independent experiments using 3 separate batches of saRNA.

Referring to FIG. 8, there is shown f-Luc expression in HeLa cells following transfection with selected VEEV replicons containing IIP in F-T2A configuration relative to expression in HEK293T/17 cells. Details of experimental methods are provided in FIG. 6. PIV V5 and MERS ORF4a produced the greatest increase inf-Luc expression. Data shown are constructs providing a greater than 2-fold increase in luciferase expression in HeLa cells relative to expression in HEK293T/17 cells and are mean f SEM of data obtained in 3 independent experiments using 3 separate batches of saRNA.

Referring to FIG. 9, there is shown f-Luc expression in HeLa cells following transfection with VEEV replicons containing IIP in a double sub-genomic promoter (DSGP) configuration relative to expression in HEK293T/17 cells. Details of experimental methods are provided in FIG. 6. HCV E2, VACV E3L and PIV 5V produced similar increases in f-Luc expression. Data shown are constructs providing a greater than 2-fold increase in luciferase expression in HeLa cells relative to expression in HEK293T/17 cells and are mean f SEM of data obtained in 3 independent experiments using 3 separate batches of saRNA.

Referring to FIG. 10, there is shown the increase in VEGF-A secretion from HeLa cells following transfection with saRNA containing the IIP HSV ICP34.5 in a F-T2A configuration compared to saRNA without IIP and relative to expression in HEK293T/17 cells. HEK293T/17 and HeLa cells were transfected with RNA (100 ng) containing VEGF-A as a reporter protein and assessed for protein expression and secretion into the culture media after 48 hr by ELISA. HeLa cells are known to have more intact IFN expression pathways compared to HEK293T/17 and therefore increased expression relative to a control (RNA containing VEGF-A as GOI and no IIP) indicates that HSV ICP34.5 increases saRNA GOI expression. Data are from one experiment and represent the mean f SEM of three replicate measurements.

Referring to FIG. 11, there is shown f-Luc expression in HeLa cells following transfection with saRNA containing f-Luc as the GOI (construct 1) and with the IIP MERS ORF4a in F-T2A configuration (constructs 2a and 2b), IRES configuration (construct 3b) and DSGP configurations (constructs 4a and 4b) shown in FIG. 1. Details of experimental methods are provided in FIG. 6. Data are mean±SEM of data obtained in 3 independent experiments using 3 separate batches of saRNA. P<0.05 repeated measures ANOVA compared to construct with no IIP.

### Example 3—Construction and Testing RNA Constructs Comprising a Viral Innate Inhibitor Protein (IIP)

The inventors designed, constructed and then tested a series of diverse viral IIPs, and the results of the expression studies are shown in FIG. 12.

Referring to FIG. 12, there is shown n-Luc expression in HeLa cells following transfection with RNA containing IIP in F-T2A configuration relative to expression in HEK293T/17 cells. Details of experimental methods are provided in FIG. 6. Data shown are constructs providing a greater than 2-fold increase in luciferase expression and are mean±SEM of data obtained in 3 independent experiments using 3 separate batches of RNA.

### CONCLUSIONS

The inventors believe that the constructs described herein display many advantages over those described in the prior art, including:
i) insertion of nucleotide sequences encoding any of the innate modulatory proteins directly into the RNA construct, such as mRNA or saRNA, enabling dual protein expression of the IIP protein and the biotherapeutic molecule encoded by the gene of interest;
ii) as opposed to delivering two different and separate strands of RNA, one encoding the gene of interest (GOI), i.e. the therapeutic biomolecule, and one encoding the IIP, a single strand is required to be delivered;
iii) the IIP inhibits innate sensing of RNA, thus enabling higher protein expression;
iv) when the RNA construct is a saRNA, the IIP expression itself is self-amplified by virtue of being co-expressed on the sub-genome strand with the GOI; and/or
v) an increase in both the magnitude and duration of protein expression compared to conventional VEEV RNA replicon constructs.

Numbered Paragraphs

The following paragraphs form part of the description and not the claims
1. An RNA construct encoding: (i) at least one therapeutic biomolecule; and (ii) at least one viral innate inhibitor protein (IIP).
2. The RNA construct according to paragraph 1, wherein the construct is an mRNA, saRNA or a trans-replicon system, most preferably saRNA.
3. The RNA construct according to either paragraph 1 or paragraph 2, wherein the construct comprises or is derived from a positive stranded RNA virus selected from the group of genus consisting of: alphavirus; picornavirus; flavivirus; rubivirus; pestivirus; hepacivirus; calicivirus and coronavirus, preferably an alphavirus, optionally VEEV.
4. The RNA construct according to any preceding paragraph, wherein the at least one innate inhibitor protein (IIP) is HPV E6 or HSV ICP34.5, or an orthologue thereof.
5. The RNA construct according to any one of paragraphs 1-3, wherein the at least one innate inhibitor protein (IIP) is HCV E2 or HCV NS5a, or an orthologue thereof.
6. The RNA construct according to any one of paragraphs 1-3, wherein the at least one innate inhibitor protein (IIP) is VACV E3L or VACV K3L, or an orthologue thereof.
7. The RNA construct according to any one of paragraphs 1-3, wherein the at least one innate inhibitor protein (IIP) is MERS ORF8B, or an orthologue thereof.
8. The RNA construct according to any one of paragraphs 1-3, wherein the at least one innate inhibitor protein (IIP) is KSHV ORF52, or an orthologue thereof.
9. The RNA construct according to any one of paragraphs 1-3, wherein the at least one innate inhibitor protein (IIP) is Ebola VP35, or an orthologue thereof.
10. The RNA construct according to any one of paragraphs 1-3, wherein the at least one innate inhibitor protein (IIP) is Vaccinia C6, or an orthologue thereof.
11. The RNA construct according to any one of paragraphs 1-3, wherein the at least one innate inhibitor protein (IIP) is an ORF3b*57 variant of the wild type of SARS-CoV-2 ORF3b, or an orthologue thereof.
12. The RNA construct according to any one of paragraphs 1-3, wherein the at least one innate inhibitor protein (IIP) is an ORF3b*79 variant of the wild type of SARS-CoV-2 ORF3b, or an orthologue thereof.
13. The RNA construct according to any one of paragraphs 1-3, wherein the at least one innate inhibitor protein (IIP) is an ORF3b*57 Ecuador variant of the wild type of SARS-CoV-2 ORF3b, or an orthologue thereof.
14. The RNA construct according to any preceding paragraph, wherein the therapeutic biomolecule comprises a therapeutic protein, preferably the protein or peptide is an antigen, and more preferably a viral antigen.
15. A nucleic acid sequence encoding the RNA construct according to any preceding paragraph.
16. An expression cassette comprising a nucleic acid sequence according to paragraph 15.
17 A recombinant vector comprising the expression cassette according to paragraph 16.
18. A pharmaceutical composition comprising the RNA construct according to any one of paragraphs 1 to 14, the nucleic acid sequence according to paragraph 15, the expression cassette according to paragraph 16 or the vector according to paragraph 17, and a pharmaceutically acceptable vehicle.
19. A method of preparing the RNA construct according to any one of paragraphs 1 to 14, the method comprising:
   a) i) introducing, into a host cell, the vector according to paragraph 17; and
   ii) culturing the host cell under conditions to result in the production of the RNA construct according to any one of paragraphs 1 to 14; or
   b) transcribing the RNA construct from the vector according to paragraph 17.
20. The RNA construct according to any one of paragraphs 1 to 14, the nucleic acid sequence according to paragraph 15, the expression cassette according to paragraph 16 or the vector according to paragraph 17 or the pharmaceutical composition according to paragraph 18, for use as a medicament or in therapy.
21. The RNA construct according to any one of paragraphs 1 to 14, the nucleic acid sequence according to paragraph 15, the expression cassette according to paragraph 16 or the vector according to paragraph 17 or the pharmaceutical composition according to paragraph 18, for use in the prevention, amelioration or treatment of a protozoan, fungal, bacterial or viral infection.
22. The RNA construct according to any one of paragraphs 1 to 14, the nucleic acid sequence according to paragraph 15, the expression cassette according to paragraph 16, the vector according to paragraph 17 or the pharmaceutical composition according to paragraph 18, for use in the prevention, amelioration or treatment of cancer.
23. A vaccine comprising the RNA construct according to any one of paragraphs 1 to 14, the nucleic acid sequence according to paragraph 15, the expression cassette according to paragraph 16, the vector according to paragraph 17 or the pharmaceutical composition according to paragraph 18.
24. The RNA construct according to any one of paragraphs 1 to 14, the nucleic acid sequence according to paragraph 15, the expression cassette according to paragraph 16, the vector according to paragraph 17 or the pharmaceutical composition according to paragraph 18, for use in stimulating an immune response in a subject.

1. An RNA construct encoding: (i) at least one therapeutic biomolecule; and (ii) at least one viral innate inhibitor protein (IIP).

2. The RNA construct according to claim 1, wherein the construct is a mRNA molecule.

3. The RNA construct according to claim 1, wherein the construct is a saRNA molecule.

4. The RNA construct according to claim 1, wherein the construct comprises or is derived from a positive stranded RNA virus selected from the group of genus consisting of: alphavirus; picornavirus; flavivirus; rubivirus; pestivirus; hepacivirus; calicivirus and coronavirus, preferably an alphavirus, optionally VEEV.

5. The RNA construct according to claim 1, wherein the at least one innate inhibitor protein (IIP) is:
(i) HPV16 E6, or an orthologue thereof,
(ii) HSV ICP34.5, or an orthologue thereof,
(iii) HCV E2, or an orthologue thereof,
(iv) HCV NS5a, or an orthologue thereof,
(i) VACV E3L, or an orthologue thereof,
(ii) VACV K3L, or an orthologue thereof,
(iii) MERS ORF8B, or an orthologue thereof,
(iv) KSHV ORF52, or an orthologue thereof, and/or
(v) Ebola VP35, or an orthologue thereof.

6. The RNA construct according to claim 1, wherein the at least one innate inhibitor protein (IIP) is Vaccinia C6, or an orthologue thereof.

7. The RNA construct according to claim 1, wherein the at least one innate inhibitor protein (IIP) is EV71-2Apro, or an orthologue thereof.

8. The RNA construct according to claim 1, wherein the at least one innate inhibitor protein (IIP) is BVDV nPro, or an orthologue thereof.

9. The RNA construct according to claim 1, wherein the at least one innate inhibitor protein (IIP) is HSV Us1, or an orthologue thereof.

10. The RNA construct according to claim 1, wherein the at least one innate inhibitor protein (IIP) is Simian Virus 5 (PIV5 Non-structural protein V), or an orthologue thereof.

11. The RNA construct according to claim 1, wherein the at least one innate inhibitor protein (IIP) is (i) an ORF3b*57 variant of the wild type of SARS-CoV-2 ORF3b, or an orthologue thereof, or (ii) an ORF3b*57 Ecuador variant of the wild type of SARS-CoV-2 ORF3b, or an orthologue thereof.

12. The RNA construct according to claim 1, wherein the at least one innate inhibitor protein (IIP) is: (i) an ORF3b*57 Pangolin variant of the wild type of SARS-CoV-2 ORF3b, or an orthologue thereof, or (ii) an ORF3b*79 variant of the wild type of SARS-CoV-2 ORF3b, or an orthologue thereof.

13. The RNA construct according to claim 1, wherein the at least one innate inhibitor protein (IIP) is an ORF3b*79 Pangolin variant of the wild type of SARS-CoV-2 ORF3b, or an orthologue thereof.

14. The RNA construct according to claim 1, wherein the therapeutic biomolecule comprises a therapeutic protein, preferably the protein or peptide is an antigen, and more preferably a viral antigen.

15. A nucleic acid sequence encoding the RNA construct according to claim 1.

16-24. (canceled)
